(19) United States
(12) Patent Application Publication
(10) Pub. No.: US 2026/0347803 A1
(43) Pub. Date: Nov. 5, 2026

(54) CONSTRUCTS RELATING TO GENETIC DISORDERS

(71) Applicant: Pacific Northwest Research Institute, Seattle, WA (US)

(72) Inventors: Michael J. Xie, Seattle, WA (US); Gareth A. Cromie, Seattle, WA (US); Aimee M. Dudley, Seattle, WA (US)

(21) Appl. No.: 19/124,213

(22) PCT Filed: Nov. 2, 2023
(86) PCT No.: PCT/US2023/078549

§ 371 Date: Apr. 24, 2025

Related U.S. Application Data
(60) Provisional application No. 63/382,438, filed on Nov. 4, 2022.

(51) Int. Cl. C12N 9/10; A61K 38/00; C12Q 1/52

(52) U.S. Cl. CPC C12N 9/1096; C12Q 1/52; C12Y 206/01052; A61K 38/00

(57) ABSTRACT

Polynucleotides, expression constructs, compositions, kits and method relating to genetic disorders are disclosed. Some embodiments provided herein encompass variants of a protein that relate to a genetic disorder. Also contemplated are expression constructs that encode highly functional and/or stable protein variants. Also described herein are expression constructs that encode protein variants with reduced activity and/or stability. Also described herein are compositions, kits and methods for diagnosing and treating the genetic disorder. In some embodiments, the variants can relate to the treatment and/or diagnosis of genetic disorders.

### CROSS REFERENCE TO RELATED APPLICATION

This application claims priority to U.S. Provisional Patent Application No. 63/382,438, filed Nov. 4, 2022, the entire contents of which is incorporated by reference herein.

### STATEMENT REGARDING FEDERALLY SPONSORED R&D

This invention was made with government support under Grant No. R01 GM134274 awarded by the National Institutes of Health/National Institute of General Medical Sciences. The government has certain rights in the invention.

### FIELD

The present disclosure relates to polynucleotides, expression constructs, compositions, kits and methods relating to genetic disorders.

### REFERENCE TO SEQUENCE LISTING

The present application is being filed along with a Sequence Listing in electronic format. The Sequence Listing is provided as a file entitled PNDRI.015WO.xml, which was created on Oct. 25, 2023, which is 1,899,795 bytes in size. The information in the electronic Sequence Listing is hereby incorporated by reference in its entirety.

### BACKGROUND

Inborn errors of metabolism (IEMs) are a class of genetic diseases that can give rise to debilitating conditions if left untreated. These diseases present a broad range of phenotypes including lethality, severe neurological manifestations, seizures, and intellectual disability.

### SUMMARY

This application is related to protein variants of PSAT1 that harbor different amino acid substitutions. Any features, structures, or steps disclosed herein can be replaced with or combined with any other features, structures, or steps disclosed herein, or omitted. Further, for purposes of summarizing the disclosure, certain aspects, advantages, and features of the inventions have been described herein. It is to be understood that not necessarily any or all such advantages are achieved in accordance with any particular embodiment of the inventions disclosed herein. No individual aspects of this disclosure are essential or indispensable.

In some embodiments, a protein with the amino acid sequence any one of the SEQ ID NOS: 1408-1537 is provided. In some embodiments, a protein that is at least 80% identical to SEQ ID NO: 1 (WT Sequence) is provided, including one or more of the amino acid substitution mutations in Table 2. In some embodiments, the protein with at least one of the amino acid substitution mutations in Table 2 exhibits higher activity when compared to the WT protein of SEQ ID NO: 1. In some embodiments, the function of the protein with at least one of the amino acid substitution mutations in Table 2 is enhanced greater than 5% when compared to the function of WT protein of SEQ ID NO: 1. In some embodiments, the function of the protein with at least one of the amino acid substitution mutations in Table 2 is enhanced by at least 10% when compared to the function of WT protein of SEQ ID NO: 1.

In some embodiments, the amino acid substitution mutants include at least one amino acid change at the amino acid positions of SEQ ID NO: 1, selected from the group consisting of 2, 3, 4, 5, 7, 8, 12, 15, 17, 22, 24, 26, 29, 31, 37, 42, 46, 48, 49, 53, 55, 56, 63, 68, 71, 72, 73, 74, 80, 85, 93, 94, 95, 97, 106, 109, 110, 116, 117, 121, 125, 132, 133, 136, 138, 144, 146, 148, 157, 169, 174, 180, 193, 198, 203, 219, 221, 226, 229, 231, 236, 239, 240, 242, 243, 245, 249, 250, 253, 255, 259, 284, 300, 302, 310, 311, 318, 325, 334, 336, 341, 344, 349, 350, 351, 353, 354, 356, 357, 359, 369. In some embodiments, the amino acid substitution mutants include at least one amino acid change at the amino acid positions of SEQ ID NO:1, selected from the group consisting of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 118, 119, 120, 121, 122, 123, 124, 125, 126, 127, 128, 129, 130, 132, 133, 134, 135, 136, 137, 138, 139, 140, 141, 142, 143, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370.

In some embodiments, an expression vector including a polynucleotide sequence encoding for any of the embodiments of the present application is provided. In some embodiments, an expression vector including a polynucleotide sequence encoding one or more proteins of SEQ ID NOS: 1408-1537 is provided. In some embodiments, the sequence is a codon-optimized sequence. In some embodiments, the polynucleotide sequence includes one or more of the amino acid substitution mutations from Table 2 that is enhanced greater than 5% when compared to the function of wild-type protein of SEQ ID NO:1. In some embodiments, the protein is phosphoserine aminotransferase (PSAT1).

In some embodiments, a composition comprising the protein of any one of the SEQ ID NOS: 1408-1537 is provided. In some embodiments, the composition is for use in the treatment of a genetic disorder. In some embodiments, the composition is administered to a subject having a genetic disorder in an amount effective to correct the genetic disorder or a symptom thereof in the subject. In some embodiments, the genetic disorder is phosphoserine aminotransferase (PSAT1) deficiency (PSATD). In some embodiments, the genetic disorder is Neu-Laxova Syndrome 2 (NLS2). In some embodiments, PSATD, or a symptom thereof, is reduced after the administration. In some embodiments, NLS2, or a symptom thereof, is reduced after the administration. In some embodiments, the subject is a fetus, neonate, juvenile or adult. In some embodiments, one or more expression vectors is administered to a subject in an amount effective to correct the PSATD or a symptom thereof in the subject. In some embodiments, one or more expression vectors is administered to a subject in an amount effective to correct the NLS2 or a symptom thereof in the subject. In some embodiments, the PSAT1 is modified to exhibit an enhanced function. In some embodiments, the PSAT1 is replaced or supplemented by one or more polynucleotide sequences encoding SEQ ID NOS: 4-1537 to exhibit an enhanced function.

In some embodiments, the function of the protein with at least one of the amino acid substitution mutations in Table 2 is enhanced greater than 5% when compared to the function of WT protein of SEQ ID NO: 1. In some embodiments, the function of the protein with at least one of the amino acid substitution mutations in Table 2 is enhanced by at least 50% when compared to the function of WT protein of SEQ ID NO:1.

In some embodiments, a kit for determining a specific mode of treatment of a genetic disorder is provided, the kit including: a look up table, wherein the look up table indicates specific amino acid substitutions that are commonly involved in a cellular mechanism; and one or more vectors including sequences of SEQ ID NOS: 4-1537. In some embodiments, the look up table includes a list of mutations from Tables 2 and 3. In some embodiments of the kit, the genetic disorder is PSATD. In some embodiments of the kit, the genetic disorder is NLS2. In some embodiments, the look up table includes a list of mutations from Tables 3 (having decreased activity, in need of increased activity). In some embodiments, the look up table includes a list of mutations from Tables 2 (which could be used to identify sequences useful for enhancing activity).

In some embodiments, a method of identifying a subject having a genetic disorder is provided, the method including detecting a presence of at least one mutation in the protein that is different in the subject with a genetic disorder as compared to a subject without the disorder. In some embodiments, the presence of at least one mutation indicates the presence of a genetic disorder in the subject. In some embodiments, the genetic disorder or a symptom thereof is reduced after administration. In some embodiments, the mutation is selected from a group consisting of mutations listed in Table 3. In some embodiments, the genetic disorder is PSATD. In some embodiments, the one mutation that indicates the presence of genetic disorder in the subject is selected from the mutations in Table 3. In some embodiments, an engineered protein administered to the subject includes one or more of the mutations in Table 2.

In some embodiments, a method of improving, ameliorating, or treating a genetic disorder is provided, the method including: receiving diagnostic information, the diagnostic information including a detection of a presence of a DNA variant that results in at least one mutation in a protein that is different in the subject with a genetic disorder as compared to a subject without the disorder, wherein the presence of at least one mutation indicates the presence of the genetic disorder in the subject; administrating to the subject a treatment, wherein the genetic disorder or a symptom thereof is reduced after administration. In some embodiments, the treatment includes an effective amount of a dietary supplementation.

In some embodiments, the dietary supplementation includes L-serine. In some embodiments, the effective amount includes 100 to 950 mg/kg/day of the L-serine. In some embodiments, the effective amount includes 400 to 950 mg/kg/day of the L-serine. In some embodiments, the effective amount includes 500 mg/kg/day of the L-serine. In some embodiments, the dietary supplementation includes glycine. In some embodiments, the effective amount includes 200 mg/kg/day of the glycine. In some embodiments, the effective amount includes 400 mg/kg/day of the glycine. In some embodiments, the effective amount includes 200 to 400 mg/kg/day of the glycine. In some embodiments, the dietary supplementation includes L-serine and glycine. In some embodiments, the effective amount includes 500 mg/kg/day of the L-serine and 200 mg/kg/day of the glycine. In some embodiments, the effective amount includes 300 mg/kg/day of the L-serine and 400 mg/kg/day of the glycine. In some embodiments, the effective amount includes: 300 to 500 mg/kg/day of the L-serine; and 200 to 400 mg/kg/day of the glycine. In some embodiments, the genetic disorder is PSATD. In some embodiments, the genetic disorder is NLS2. In some embodiments, the one mutation that indicates the presence of the genetic disorder in the subject is selected from the mutations in Table 3.

In some embodiments, a method of improving, ameliorating, or treating a disorder, the method including: receiving diagnostic information, the diagnostic information including a detection of a presence of a DNA variant that results in at least one mutation in a protein that is different in the subject with a disorder as compared to a subject without the disorder, wherein the presence of at least one mutation indicates the presence of the disorder in the subject; administrating to the subject a treatment, wherein the disorder or a symptom thereof is reduced after administration, wherein the disorder is associated with reduced activity in serine biosynthesis. In some embodiments, the disorder includes ichthyosis. In some embodiments, the disorder includes epilepsy. In some embodiments, the disorder includes hypertension. In some embodiments, the disorder includes retinal degeneration. In some embodiments, the disorder includes macular telangiectasia type 2. In some embodiments, the mutation protein includes an amino acid substitution of at least one amino acid of WT PSAT1. In some embodiments, the treatment includes an effective amount of a dietary supplementation.

In some embodiments, the dietary supplementation includes L-serine. In some embodiments, the effective amount includes 100 to 950 mg/kg/day of the L-serine. In some embodiments, the effective amount includes 400 to 950 mg/kg/day of the L-serine. In some embodiments, the effective amount includes 500 mg/kg/day of the L-serine. In some embodiments, the dietary supplementation includes glycine. In some embodiments, the effective amount includes 200 mg/kg/day of the glycine. In some embodiments, the effective amount includes 400 mg/kg/day of the glycine. In some embodiments, the effective amount includes 200 to 400 mg/kg/day of the glycine. In some embodiments, the dietary supplementation includes L-serine and glycine. In some embodiments, the effective amount includes 500 mg/kg/day of the L-serine and 200 mg/kg/day of the glycine. In some embodiments, the effective amount includes 300 mg/kg/day of the L-serine and 400 mg/kg/day of the glycine. In some embodiments, the effective amount includes: 300 to 500 mg/kg/day of the L-serine; and 200 to 400 mg/kg/day of the glycine.

### BRIEF DESCRIPTION OF THE DRAWINGS

The foregoing and other features of the present disclosure will become more fully apparent from the following description, taken in conjunction with the accompanying drawings. Understanding that these drawings depict only some embodiments in accordance with the disclosure and are therefore, not to be considered limiting of its scope, the disclosure will be described with additional specificity and detail through use of the accompanying drawings.

FIG. 1 illustrates the sequence of human PSAT1 protein set forth in GenBank accession No: NP_478059.1.

FIG. 2 illustrates the sequence of the human PSAT1 protein where amino acid positions that can be substituted in the sequence are depicted with X.

FIG. 3 illustrate human PSAT1 protein where amino acid positions that can be substituted in the sequence are depicted with X.

FIG. 4 illustrate the sequence of human PSAT1 protein variant of SEQ ID NO: 3-1407.

FIG. 5 illustrate the sequence of human PSAT1 protein variant of SEQ ID NO: 1408-1537.

FIG. 6 illustrate the sequence of the expression construct set forth in GenBank accession No: MN654101.

FIG. 7 shows the results of a quantitative yeast assay for PSAT1 function in the absence of serine. Growth in the absence of exogenously supplied serine, for wildtype strain (codon optimized human PSAT1 gene) and strains with amino acid substitution mutations in human PSAT1. The growth values (y-axis) plotted as points on a scatter plot ordered by their amino acid position (x-axis).

FIG. 8 show results of dietary supplementation across various cases in treating conditions manifested from at least one mutation in the human PSAT1 gene (adapted from Shen et al., “Juvenile-onset PSAT1-related neuropathy: A milder phenotype of serine deficiency disorder” Front Genet. 2022 Aug. 16; 13:949038 (PMID: 36061210)).

### DETAILED DESCRIPTION

While dietary restriction or supplementation can have a positive impact on patient health, timely recognition and targeted treatments are crucial. However, many IEMs exhibit common symptoms but require radically different treatments. Serine deficiency disorders are examples of IEMs that are highly actionable. Serine is central to processes such as cellular proliferation, folate metabolism, phospholipid metabolism, and the formation of neuromodulators D-serine and glycine. Deficiency in serine can have profound effects on central nervous system development. PSAT1 deficiency is caused by mutations in the gene that encodes phosphoserine aminotransferase (PSAT1), which is the second enzyme in the serine biosynthesis pathway. This rare but highly actionable disease can lead to severe conditions such as Neu-Laxova Syndrome 2, a condition that is characterized by a recognizable pattern of severe malformations that lead to prenatal or postnatal lethality.

Existing biochemical assays for PSAT1 deficiency have limitations, including the assessment of serine levels, which generally requires invasive measuring metabolite concentrations in cerebrospinal fluid and comparison to closely age-matched controls. Computational methods for predicting the effect of variants from polymorphisms often yield inaccurate or conflicting results. Therefore, DNA sequencing-based diagnostics have the potential not to only reduce the morbidity and mortality associated with delayed treatment or underdiagnosis in various diseases including IEMs. However, because approximately 25% of PSAT1 deficiencies occur de novo, and because only a minority of PSAT1 variants are recurrent, DNA sequencing often returns novel sequence variants of uncertain significance (VUS).

The currently existing biochemical assays for detecting PSAT1 deficiency (PSATD) are enzymatic assays. These assays have limitations, including the sensitivity and specificity of these assays used in newborn screening panels. In addition, only a minority of PSAT1 variants are recurrent and DNA sequencing-based diagnostics often returns novel sequence variants of uncertain significance (VUS).

Identification of protein variants with specific attributes can be used to stratify patients for specific therapeutics. Because it is not currently possible to computationally predict the functional consequences of all amino acid substitution mutations, results from functional assays, such as those described here for PSAT1, are needed to accurately and quantitatively determine the degree to which an amino acid substitution will (or will not) alter a protein's activity, stability, or both.

Provided herein are protein variants of PSAT1. In some embodiments, they can be for applications in which protein variants with increased activity and/or stability are desired. Also disclosed herein are protein variants with reduced activity and/or stability that can be used to diagnose PSATD. In some embodiments, protein variants with increased activity and/or stability can be used to replace or supplement the protein variants with reduced activity and/or stability-in some embodiments, this identification can then be used to then treat the subject as provided herein (e.g., via an increase in activity and/or function of PSAT1).

In some embodiments, a method of improving, ameliorating, or treating a genetic disorder is provided. The method comprises detecting the presence of at least one mutation that results in the protein variant with reduced activity and/or stability that is different in the subject with genetic disorder as compared to a subject without the disorder, wherein the presence of at least one mutation that results in the protein variant with reduced activity and/or stability indicates the presence of genetic disorder in the subject. The method further comprises administering a composition comprising at least one protein variant with increased activity and/or stability to the subject, wherein the genetic disorder or a symptom thereof is reduced after administration.

### TERMINOLOGY

Unless defined otherwise, all technical and scientific terms used herein have the same meaning as commonly understood by one of ordinary skill in the art to which the invention pertains.

As used herein, “a” or “an” may mean one or more than one.

“About” as used herein when referring to a measurable value is meant to encompass variations of ±20% or ±10%, more preferably ±5%, even more preferably ±1%, and still more preferably ±0.1% from the specified value.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The ranges disclosed herein also encompass any and all overlap, sub-ranges, and combinations thereof. Language such as “up to,” “at least,” “greater than,” “less than,” “between,” and the like includes the number recited.

Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

“Polynucleotide,” as described herein refers to “nucleic acid” or “nucleic acid molecule,” such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally occurring or modified nucleic acid bases attached to a polyamide backbone. Nucleic acids can be either single stranded or double stranded. In some alternatives, a nucleic acid sequence encoding a fusion protein is provided. In some alternatives, the nucleic acid is RNA or DNA.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 residues long, or any length in a range defined by any two of the aforementioned lengths.

“Hyperactive variant” as described herein refers to a protein with at least one amino acid substitution that has increased activity as compared to wild type or native protein. Hyperactive variant exhibits an enhanced functionality, abundance, activity, specificity, affinity or a combination of the aforementioned parameters as compared to wild type or native protein.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., an .alpha.-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds that have the same basic chemical structure as a naturally occurring amino acid, i.e., an .alpha.-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

“Expression construct” or “construct” or “an expression vector” or a “vector” as described herein, is a nucleic acid used to introduce heterologous nucleic acids into a cell that has regulatory elements to provide expression of the heterologous nucleic acids in the cell. Typically, an expression vector comprises a transcription promoter, a gene, and a transcription terminator. Gene expression is usually placed under the control of a promoter, and such a gene is said to be “operably linked to” the promoter. Similarly, a regulatory element and a core promoter are operably linked if the regulatory element modulates the activity of the core promoter. The constructs described herein include but are not limited to plasmid, minicircles, yeast, and viral genomes.

A single copy of the construct is inserted stably into the yeast genome (at either the same position as the yeast gene, which has been deleted or at a neutral position in the genome, e.g. the HO locus). In the embodiments herein, the construct is a “linear DNA molecule (double or single stranded) that can be integrated into the yeast nuclear of mitochondrial genome by homologous recombination. These constructs may be synthesized in vitro by a commercial gene synthesis company (e.g. Twist Biosciences or IDT). As such the nucleic acid sequence could be the human sequence or a yeast optimized version that encodes the same protein sequence.

“Coding for” or “encoding” have their plain and ordinary meaning when read in light of the specification, and may include but is not limited to, for example, the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (for example, 70%, 75%, 80%, 85%, 90%, 95%, 96%, 97%, 98% or 99% sequence identity over a specified region, or, when not specified, over the entire sequence of a reference sequence), when compared and aligned for maximum correspondence over a comparison window, or designated region as measured using one of the following sequence comparison algorithms or by manual alignment and visual inspection. Some embodiments provided herein provide polypeptides or polynucleotides that are substantially identical to the polypeptides or polynucleotides, respectively, exemplified herein. Optionally, the identity exists over a region that is at least about 15, 25 or 50 nucleotides in length, or more preferably over a region that is 100 to 500 or 1000 or more nucleotides in length, or over the full length of the reference sequence. With respect to amino acid sequences, identity or substantial identity can exist over a region that is at least 5, 10, 15 or 20 amino acids in length, optionally at least about 25, 30, 35, 40, 50, 75 or 100 amino acids in length, optionally at least about 150, 200 or 250 amino acids in length, or over the full length of the reference sequence. With respect to shorter amino acid sequences, e.g., amino acid sequences of 20 or fewer amino acids, in some embodiments, substantial identity exists when one or two amino acid residues are conservatively substituted, according to the conservative substitutions defined herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

The terms “individual”, “subject”, “host,” or “patient” as used herein have their usual meaning as understood by those skilled in the art and thus includes a human or a non-human mammal. The term “mammal” is used in its usual biological sense. Thus, it specifically includes, but is not limited to, primates, including simians (chimpanzees, apes, monkeys), humans, cattle, horses, sheep, goats, swine, rabbits, dogs, cats, rodents, rats, mice, or guinea pigs. The subject as described in the embodiments herein may be a fetus, neonate, juvenile or adult. “Fetus” has its plain and ordinary meaning when read in light of the specification, and may include but is not limited to, for example, a prenatal human between its embryonic state and its birth. “Neonate” refers to a newborn child. “Juvenile” refers to a person under the established age of 18 years. “Adult” refers to a person any age over 18.

“Look-up table” has its plain and ordinary meaning when read in light of the specification, and may include but is not limited to, for example, a table prepared with a list or combination of protein variants that may exhibit increased or decreased levels of activity and/or stability. The data on the look-up table also provide different predicted thresholds for determining the functions of different amino acid substitution mutations as there are different thresholds for combinations of alleles for different diseases.

“Cell growth” also referred to as the “growth value”, is an experimentally measured amount of the extent to which yeast cells are able to grow under specific environmental conditions. In some embodiments, cell growth, also the “growth value”, is measured by optical density of a liquid culture. In some embodiments, cell growth, also the “growth value”, is measured by the area of a “patch” or “spot” of cells growing on solid media, the number of pixels of a “patch” or “spot” of cells growing on solid media, the intensity of the pixels in a “patch” or “spot” of cells growing on solid media, the change in any of these parameters over time, or a combination of any of the aforementioned parameters.

As used herein, by “pharmaceutically acceptable” or “pharmacologically compatible” is meant a material that is not biologically or otherwise undesirable, e.g., the material may be incorporated into a pharmaceutical composition administered to a subject without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Codon optimization” or “codon optimized” refers to changes made in the nucleotide sequence so that it is more likely to be expressed at a relatively high level and translated at a higher efficiency as compared to the non-codon optimized sequence. Codon optimization does not change the amino acid for which the codon encodes.

“Codon harmonization” or “codon harmonized” refers to changes made in a nucleotide sequence that does not change the identity of the amino acid encoded by the codon. Codon harmonization preserves codon preferences of the original genes and seeks to match the codon frequency at an amino acid position between two orthologous genes.

As used herein, “treatment” or “therapy” of a disease or condition refers to reducing the severity, frequency, or occurrence of at least one symptom of that disease or condition, compared to a similar but untreated patient. Treatment can also refer to reducing, halting, slowing, or reversing the progression of a disease or condition, compared to a similar but untreated patient. Treatment may further comprise addressing the root cause of the disease and/or one or more symptoms. The term “prevent” does not require the absolute prohibition of the disorder or disease.

The terms “amount effective” “effective amount” or “effective dose” as used herein have their usual meaning as understood by those skilled in the art and refer to that amount of a recited composition or compound that results in an observable biological effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein. In the case of PSAT1 deficiency, an effective amount of protein or composition comprising a protein as disclosed herein, can increase the activity of the deficient protein involved in PSAT1 deficiency. In some embodiments, the therapeutically effective amount of protein or composition comprising a protein as disclosed herein, can increase the stability of the deficient protein involved in PSAT1 deficiency. In some embodiments, the therapeutically effective amount is an amount that extends the survival of a subject. In some embodiments, the therapeutically effective amount is an amount that reduces one or more symptoms associated with the PSAT1 deficit in the subject.

As used herein, “amino acid substitution mutation” refers to any mutation appreciated by one skilled in the art that results in a different amino acid being encoded at that position. In some embodiments, the mutation can be a point mutation (i.e., single nucleotide polymorphisms, “SNP”). In some embodiments, the mutation can be a mutation in 1, 2, or all 3 of the nucleotides that determine which amino acid is encoded at that position in the protein.

“Conservative substitutions” involve the replacement of an amino acid with a different amino acid having similar properties. For example, an aliphatic residue may be replaced by another aliphatic residue, a non-polar residue may be replaced by another non-polar residue, an acidic residue may be replaced by another acidic residue, a basic residue may be replaced by another basic residue, a polar residue may be replaced by another polar residue or an aromatic residue may be replaced by another aromatic residue. “Non-conservative substitutions” involve the replacement of an amino acid with a different amino acid having different properties. Amino acids may be grouped into different classes according to common side-chain properties: a. hydrophobic: Norleucine, Met, Ala, Val, Leu, Ile; b. neutral hydrophilic: Cys, Ser, Thr, Asn, Gln; c. acidic: Asp, Glu; d. basic: His, Lys, Arg; e. residues that influence chain orientation: Gly, Pro; aromatic: Trp, Tyr, Phe. Non-conservative substitutions will entail exchanging a member of one of these classes for another class.

In some embodiments, a protein with any one of the SEQ ID NOS: SEQ ID NO: 1408-1537 is provided.

In some embodiments, a protein that is at least 80% identical to SEQ ID NO: 1 (WT Sequence), comprising one or more of the amino acid substitution mutations as follows: K110T, P12A, N284T, I73V, V250G, N236S, A3G, K133R, V354F, T132S, I341V, G203A, V72L, F219I, V354L, V250A, I37M, A359V, K94R, L93F, I73F, V193L, V349G, S138G, F74C, S344C, K94Q, K318Q, K94T, P243A, L239M, K259T, M42I, L221I, K356Q, V8F, V7E, I351M, L255M, V72M, D136E, R336S, L22S, N236D, K169Q, A146T, A15T, L325V, I37T, Y240N, I73M, L29V, K231N, L63M, K300R, Q369K, D2Y, Y249F, K110E, H125R, E24A, S226A, C245G, I53F, T350P, P144T, G334A, R97M, R5S, N236K, P4T, T242S, Q26R, R97T, N302I, Y148F, T56I, E24K, K169E, D68E, Y249C, L17Q, E229A, L63V, P4R, I37N, C80G, L357M, A310S, N55T, P144Q, A109T, A15S, A85G, D353E, D48G, N180D, K116Q, I121T, S46A, L253S, D31Y, V157L, K116T, F49Y, K311Q, V157M, P144R, L221R, D31A, A95P, K110M, I73L, K356T, A198S, C80W, N55H, A106D, I121V, K117N, 137L, K117M, K117R, V174I, K117Q, P4H, N55K, K116E, K71Q, 137F listed in Table 2 is provided. In some embodiments, the protein has at least 80%, 85%, 90%, 95%, 96%, 97%, 98%, 99%, 100%, or any integer that is between 80 and 100%, identity to SEQ ID NO: 1 (wild type sequence). In some embodiments, the protein has at least 70%, 75%, 80%, 85%, 90%, 95%, 96%, 97%, 98%, 99%, 100%, or any integer that is between 70 and 100%, identity to SEQ ID NO: 1. In some embodiments, the protein has at least 80% identity to SEQ ID NO: 1. In some embodiments, the protein has at least 90% identity to SEQ ID NO: 1.

In some embodiments, the position of the amino acids in SEQ ID NO: 1 is based on numbering the first amino acid at the amino-terminal end of the protein as position 1 and counting up sequentially and monotonically to the carboxy-terminal amino acid. In some embodiments, the initial amino acid position is 1.

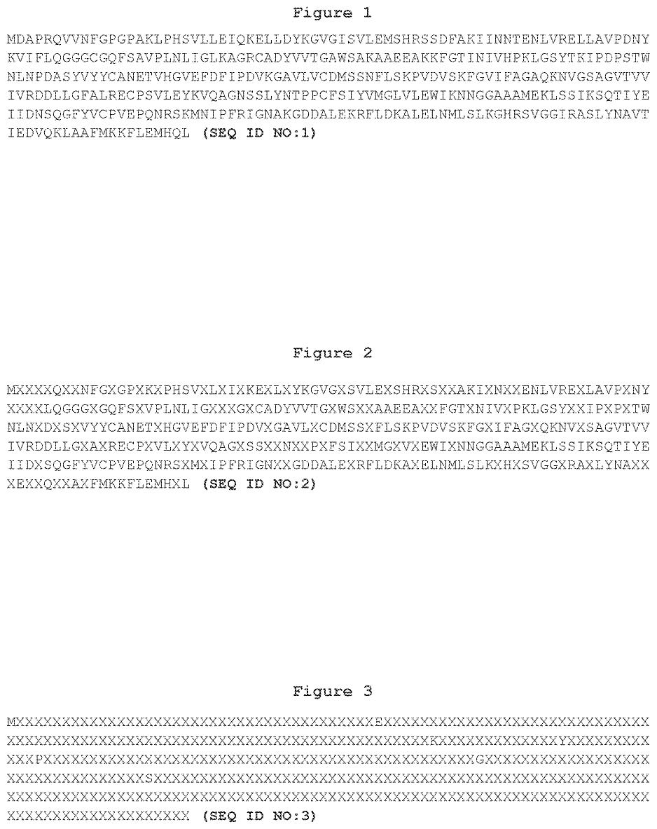

In some embodiments, the protein variants of the SEQ ID NOS: 1408-1537 can differ from a wild type sequence of FIG. 1 and SEQ ID NO: 1 by substitution of 1 amino acid, 2, 3, 4, 5-10, 10-20 or 20-30 or 30-40, 40-50, 50-60, 60-70 or 70-80 amino acids. In some embodiments, a variant sequence can comprise the wild-type sequence of FIG. 1 and SEQ ID NO: 1 with 1 residue, 2, 3, 4, 5, 6, 7, 8, 9, 10 or more residues substituted. For example, 5, 10, 15, up to 20, up to 30 or up to 40, up to 50, up to 60, up to 70, up to 80 residues can be substituted (using the substitutions specified herein, especially those that increase activity of PSAT1). In some embodiments, a variant sequence can have 1 residue, 2, 3, 4, 5, 6, 7, 8, 9, 10, 20, 30 or more residues substituted with a conservative amino acid. In other embodiments, a variant sequence can have 1 residue, 2, 3, 4, 5, 6, 7, 8, 9, 10, 20, 30 or more residues substituted with a non-conservative amino acid. In some embodiments, any one or more of the mutations listed in any one of Table 2 (or listed in Table 1 that exhibit a ratio of activity greater than 1.05) can be combined, including 2, 3, 4, 5, 6, 7, 8, 9, 10, 15, 20, 25, 30, 35, 40, 45, 50, 60, 70, 80, 90 100 or more of the amino acid substitution mutations listed in these tables.

In some embodiments, the mutants provided herein are mutants that confer increased activity and/or stability to the protein. In some embodiments, the mutants provided herein can confer an enhanced function, stability, or activity to the protein. In some embodiments, the mutants provided herein are rescue mutants. In some embodiments, the mutants provided herein can rescue the altered function, stability, or activity of the protein. In other embodiments, the mutations result in the opposite.

In some embodiments, the mutants specified herein can be used to enhance or rescue the function, stability or activity of the protein if the ratio of activity of the protein variant to wild-type protein (e.g., from Table 2) is greater than 1.05 (e.g., 1.06, 1.1, 1.15, 1.2, 1.25 or higher).

In some embodiments, the mutants specified herein can be used to enhance or rescue the function, stability or activity of the protein if the ratio of activity of the protein variant to wild-type protein is greater than 1.05.

In some embodiments, the protein with at least one of the amino acid substitution mutations listed in Table 2 exhibits higher activity when compared to the WT protein of SEQ ID NO: 1. In some embodiments, the protein with at least one of the amino acid substitutions in Table 1 exhibits higher activity when compared to the WT protein of SEQ ID NO: 1. In some embodiments, the activity of the protein can comprise but is not limited to enzymatic activity, expression levels, catalytic activity, and/or binding activity. In some embodiments, at least one or more of the amino acid substitution mutations results in a change in activity of the protein as compared to the wild-type protein. In some embodiments, the activity is at least 1%, 5% 10%, 20%, 30%, 40%, 50%, 60%, 70%, 80%, 90%, 100%, 200%, 300%, 400% or greater or any integer that is between 1 and 400%, more than the activity of the wild-type protein. In some embodiments, the ratio of activity of the protein variant to wild-type protein is 1.0 or more than 1.0. In some embodiments, the ratio of activity of the protein variant to wild-type protein is greater than 1.05 (e.g., 1.06, 1.1, 1.15, 1.2, 1.25 or higher).

In some embodiments, the function of the protein with at least one of the amino acid substitution mutations listed in Table 2 is enhanced greater than 5% when compared to the function of wild-type protein of SEQ ID NO: 1.

In some embodiments, the function of the protein with at least one of the amino acid substitution mutations listed in Table 2 is enhanced by at least 10, 20, 30, 40, 50, 100% or more when compared to the function of wild-type protein of SEQ ID NO: 1.

In some embodiments, the functionality is at least 1%, 5% 10%, 20%, 30%, 40%, 50%, 60%, 70%, 80%, 90%, 100%, 200%, 250%, 300%, 400% or any integer that is between 1 and 400%, more than the functionality of the wild-type protein. In some embodiments, the ratio of functionality of the protein variant to wild-type protein 1.0 or more than 1.0. In some embodiments, the ratio of functionality of the protein variant to wild-type protein is greater than 1.05 (e.g., 1.06, 1.1, 1.15, 1.2, or higher) as listed in Table 2.

In some embodiments, at least one or more of the amino acid substitution mutations listed in Table 2 results in a change in expression of the protein as compared to the wild-type protein. In some embodiments, the functionality is at least 1%, 5% 10%, 20%, 30%, 40%, 50%, 60%, 70%, 80%, 90%, 100%, 200%, 300%, 400% or any integer that is between 1 and 400%, more than the expression of the wild-type protein. In some embodiments, the ratio of expression of the protein variant to wild-type protein is 1 or more than 1. In some embodiments, the ratio of expression of the protein variant to wild-type protein is 1, 1.05, 1.1, 1.15, 1.2, 1,25 or higher.

In some embodiments, at least one or more of the amino acid substitution mutations listed in Table 2 results in a change in abundance of the protein as compared to the wild-type protein. In some embodiments, the abundance is at least 1%, 5% 10%, 20%, 30%, 40%, 50%, 60%, 70%, 80%, 90%, 100%, 200%, 300%, 400% or any integer that is between 1 and 400%, more than the abundance of the wild-type protein. In some embodiments, the ratio of abundance of the protein variant to wild-type protein is 1.0 or more than 1.0. In some embodiments, the ratio of abundance of the protein variant to wild-type protein is greater than 1.05 (e.g., 1.06, 1.1, 1.15, 1.2, 1.25, or higher). In some embodiments, this is measured as the mass of the amount of protein variant to wild-type protein.

In some embodiments, at least one or more of the amino acid substitution mutations results in a change in specificity of the protein as compared to the wild-type protein. In some embodiments, the specificity is at least 1%, 5% 10%, 20%, 30%, 40%, 50%, 60%, 70%, 80%, 90%, 100%, 200%, 300%, 400% or any integer that is between 1 and 400%, more than the specificity of the wild-type protein. In some embodiments, the ratio of specificity of the protein variant to wild-type protein is 1.0 or more than 1.0. In some embodiments, the ratio of specificity of the protein variant to wild-type protein is greater than 1.05 (e.g., 1.06, 1.1, 1.15, 1.2, 1.25, or higher).

In some embodiments, at least one or more of the amino acid substitution mutations results in a change in affinity of the protein as compared to the wild-type protein. In some embodiments, the abundance is at least 1%, 5% 10%, 20%, 30%, 40%, 50%, 60%, 70%, 80%, 90%, 100%, 200%, 300%, 400% or any integer that is between 1 and 400%, more than the affinity of the wild-type protein. In some embodiments, the ratio of affinity of the protein variant to wild-type protein is 1.0 or more than 1.0. In some embodiments, the ratio of affinity of the protein variant to wild-type protein is greater than 1.05 (e.g. 1.051, 1.06, 1.1, 1.15, 1.2, 1.25, 1.3, 1.35, 1.4, 1.45, 1.5, 1.55, 1.6, 1.65, 1.7, 1.75, 2, 2.5, 3, 3.5, 4, or more).

In some embodiments, amino acid substitution mutations result in a change in functionality, abundance, activity, specificity, affinity or a combination of the aforementioned parameters.

In some embodiments, the amino acid substitution mutants comprising at least one amino acid change at the amino acid positions of SEQ ID NO: 1, the amino acid positions selected from the group consisting of 2, 3, 4, 5, 7, 8, 12, 15, 17, 22, 24, 26, 29, 31, 37, 42, 46, 48, 49, 53, 55, 56, 63, 68, 71, 72, 73, 74, 80, 85, 93, 94, 95, 97, 106, 109, 110, 116, 117, 121, 125, 132, 133, 136, 138, 144, 146, 148, 157, 169, 174, 180, 193, 198, 203, 219, 221, 226, 229, 231, 236, 239, 240, 242, 243, 245, 249, 250, 253, 255, 259, 284, 300, 302, 310, 311, 318, 325, 334, 336, 341, 344, 349, 350, 351, 353, 354, 356, 357, 359, 369 is provided.

In some embodiments, the amino acid at least one position denoted by X in FIG. 2 can be substituted. In some embodiments, the amino acid in 1, 2, 3, 4, 5, 6, 7, 8, 9, 10 or more positions denoted by X can be substituted. For example, the amino acid in 5, 10, 15, up to 20, up to 30 or up to 40, up to 50, up to 60, up to 70, up to 80 positions denoted by X can be substituted. In some embodiments, the amino acid at the positions denoted by X in FIG. 2 (2, 3, 4, 5, 7, 8, 12, 15, 17, 22, 24, 26, 29, 31, 37, 42, 46, 48, 49, 53, 55, 56, 63, 68, 71, 72, 73, 74, 80, 85, 93, 94, 95, 97, 106, 109, 110, 116, 117, 121, 125, 132, 133, 136, 138, 144, 146, 148, 157, 169, 174, 180, 193, 198, 203, 219, 221, 226, 229, 231, 236, 239, 240, 242, 243, 245, 249, 250, 253, 255, 259, 284, 300, 302, 310, 311, 318, 325, 334, 336, 341, 344, 349, 350, 351, 353, 354, 356, 357, 359, 369) can be substituted by a conservative substitution. In some embodiments, the amino acid at the positions denoted by X in FIG. 2 (2, 3, 4, 5, 7, 8, 12, 15, 17, 22, 24, 26, 29, 31, 37, 42, 46, 48, 49, 53, 55, 56, 63, 68, 71, 72, 73, 74, 80, 85, 93, 94, 95, 97, 106, 109, 110, 116, 117, 121, 125, 132, 133, 136, 138, 144, 146, 148, 157, 169, 174, 180, 193, 198, 203, 219, 221, 226, 229, 231, 236, 239, 240, 242, 243, 245, 249, 250, 253, 255, 259, 284, 300, 302, 310, 311, 318, 325, 334, 336, 341, 344, 349, 350, 351, 353, 354, 356, 357, 359, 369) can be substituted by a non-conservative substitution. In some embodiments, the amino acid at the positions denoted by X in FIG. 2 (2, 3, 4, 5, 7, 8, 12, 15, 17, 22, 24, 26, 29, 31, 37, 42, 46, 48, 49, 53, 55, 56, 63, 68, 71, 72, 73, 74, 80, 85, 93, 94, 95, 97, 106, 109, 110, 116, 117, 121, 125, 132, 133, 136, 138, 144, 146, 148, 157, 169, 174, 180, 193, 198, 203, 219, 221, 226, 229, 231, 236, 239, 240, 242, 243, 245, 249, 250, 253, 255, 259, 284, 300, 302, 310, 311, 318, 325, 334, 336, 341, 344, 349, 350, 351, 353, 354, 356, 357, 359, 3690) can be substituted by a combination of conservative and non-conservative substitutions.

In some embodiments, the amino acid at least one position denoted by X in FIG. 3 can be substituted. In some embodiments, the amino acid in 1, 2, 3, 4, 5, 6, 7, 8, 9, 10, or more positions denoted by X can be substituted. For example, the amino acid in 5, 10, 15, up to 20, up to 30 or up to 40, up to 50, up to 60, up to 70, up to 80 positions denoted by X can be substituted. In some embodiments, the amino acid at the positions denoted by X in FIG. 3 (2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 118, 119, 120, 121, 122, 123, 124, 125, 126, 127, 128, 129, 130, 132, 133, 134, 135, 136, 137, 138, 139, 140, 141, 142, 143, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370) can be substituted by a conservative substitution. In some embodiments, the amino acid at the positions denoted by X in FIG. 3 (2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 118, 119, 120, 121, 122, 123, 124, 125, 126, 127, 128, 129, 130, 132, 133, 134, 135, 136, 137, 138, 139, 140, 141, 142, 143, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370) can be substituted by a non-conservative substitution. In some embodiments, the amino acid at the positions denoted by X in FIG. 3 (2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 118, 119, 120, 121, 122, 123, 124, 125, 126, 127, 128, 129, 130, 132, 133, 134, 135, 136, 137, 138, 139, 140, 141, 142, 143, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370) can be substituted by a combination of conservative and non-conservative substitutions.

In some embodiments, an expression vector which comprises a polynucleotide sequence of any one of the embodiments of the present application is provided. In some embodiments, the polynucleotide sequence is a human sequence. In other embodiments, the polynucleotide sequence is a yeast optimized version that encodes the human sequence.

In some embodiments, an expression vector comprising a polynucleotide sequence encoding for one or more of the proteins of SEQ ID NOS: 1408-1537 is provided (e.g., any with increased activity).

In some embodiments, an expression vector comprises a codon-optimized sequence. In other embodiments, the expression vector comprises a sequence that is not codon-optimized.

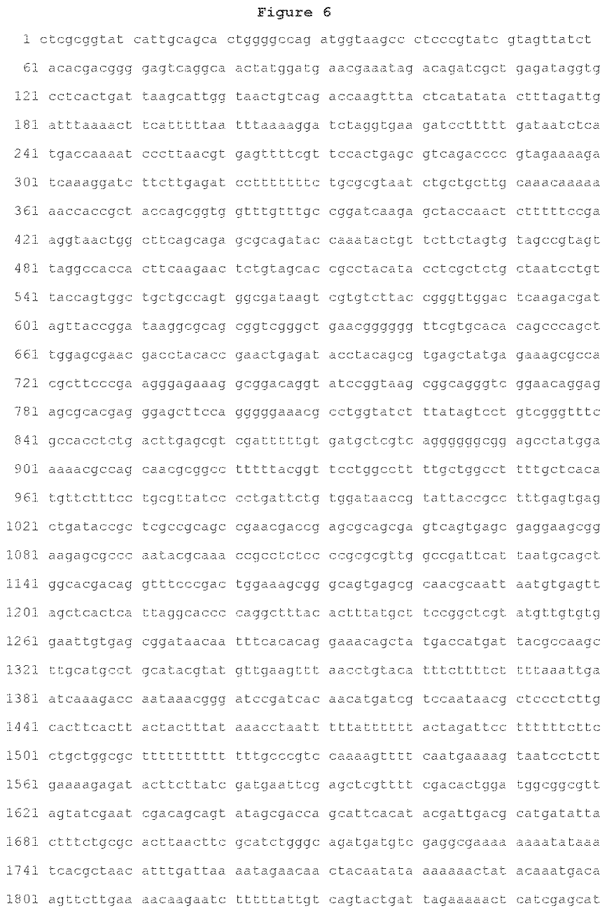

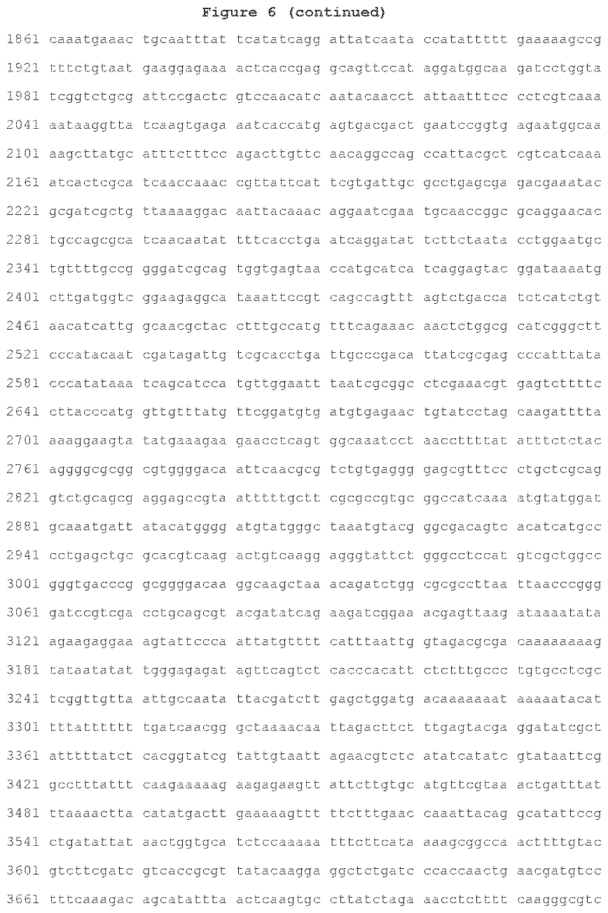

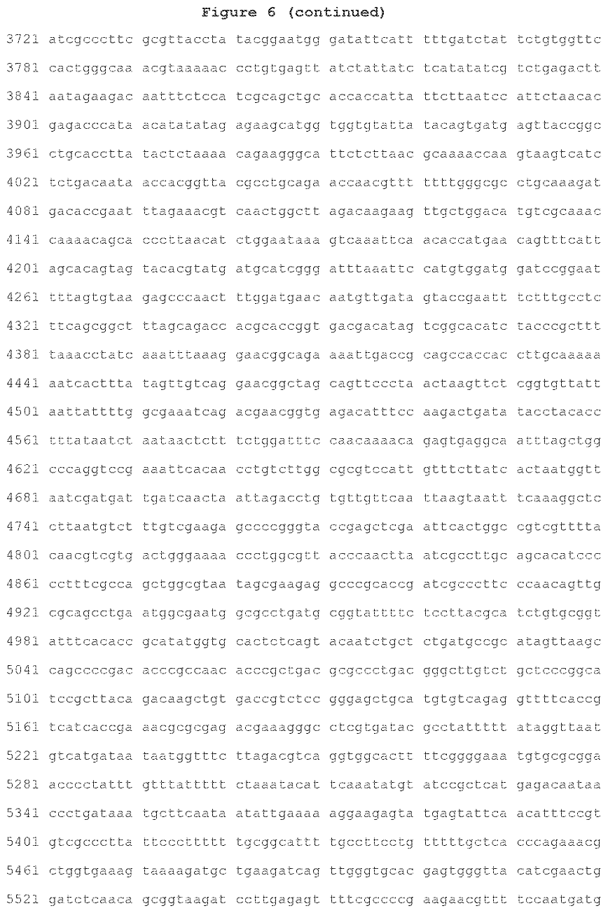

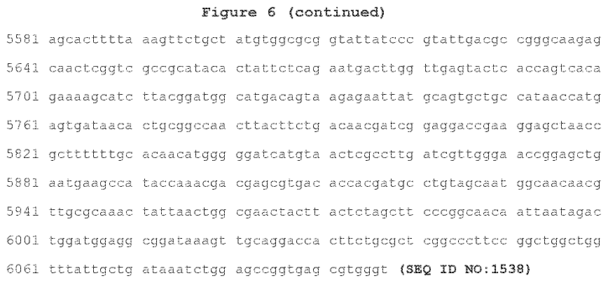

In some embodiments, the expression vector as set forth in GenBank accession No. MN654100, and as depicted in SEQ ID NO: 1538 and FIG. 6 including 1-6097=Cloning vector pAS19_yPSAT1, 1-1336=pUC19 sequence, 1337-1577=sequence derived from S. cerevisiae sequence 3′ of the SER1 (YOR184W) terminator, 1578-1839=terminator sequence derived from A. gossypii TEF, 1840-2649=KanR aminoglycoside 3′-phosphotransferase, 2650-3083=promoter sequence derived from A. gossypii TEF, 3084-3546=terminator sequence derived from S. cerevisiae 3′ of the SER1 (YOR184W) stop codon, 3547-4659=yPSAT1 derived from Homo sapiens phosphoserine aminotransferase, codon optimized for expression in S. cerevisiae, 4660-4755=derived from S. cerevisiae 5′ of SER1 (YOR184W) start codon, 4756-6097=pUC19 sequence. In some embodiments, any expression vector that can introduce a gene according to embodiments provided herein into the yeast or human genome can be used.

In some embodiments, an expression vector comprising a polynucleotide sequence encoding for one or more of the proteins of any of the embodiments comprising one or more of the amino acid substitution mutations listed in Table 2. The function of the protein variants encoded by the expression vector is enhanced greater than 5% when compared to the function of wild-type protein of SEQ ID NO: 1.

In some embodiments, the functionality is at least 1%, 5% 10%, 20%, 30%, 40%, 50%, 60%, 70%, 80%, 90%, 100%, 200%, 250%, 300%, 400% or any integer that is between 1 and 400%, more than the functionality of the wild-type protein. In some embodiments, the ratio of functionality of the protein variant to wild-type protein is 1.0 or more than 1.0. In some embodiments, the ratio of functionality of the protein variant to wild-type protein is greater than 1.05 (e.g., 1.06, 1.1, 1.15, 1.2 or higher) as listed in Table 2. In some embodiments, the ratio of functionality of the protein variant to wild-type protein is greater than 1.05 (e.g., 1.06, 1.1, 1.15, 1.2 or higher) as listed in Table 1 for the protein variants having growth estimates of greater than 1.05 (e.g., 1.06, 1.1, 1.15, 1.2 or higher).

In some embodiments, a composition comprising the protein of any one of the SEQ ID NOS: 1408-1537 is provided.

In some embodiments, a composition comprising an expression vector comprising a polynucleotide sequence encoding for one or more of the proteins of SEQ ID NOS: 1408-1537 is provided.

In some embodiments, a composition is provided comprising a protein described above and a pharmaceutically acceptable carrier. In some embodiments, a composition is provided comprising an expression vector encoding a protein according to any of the embodiments described above and a pharmaceutically acceptable carrier.

In some embodiments, the protein is phosphoserine aminotransferase (PSAT1).

In some embodiments, the composition is for use in the treatment of a genetic disorder.

In some embodiments, the composition comprising a protein variant comprising one or more amino acid substitution mutations listed in Table 2 is provided. In some embodiments, the ratio of functionality of the protein variant to wild-type protein used in the treatment of a genetic disorder exhibit is more than 1.05. For example, a protein variant with a functionality ratio of greater than 1.05 (e.g., 1.06, 1.1, 1.15, 1.2, or higher) as listed in Table 2 (or those in Table 1 having a functionality ratio greater than 1.05) can be used in the treatment of a genetic disorder. In some embodiments, two or more protein variants with the same functionality ratios can be used in the treatment of the genetic disorder. In other embodiments, two or more protein variants with different functionality ratios can be used in the treatment of the genetic disorder.

In some embodiments, a composition comprising at least one protein variant of enhanced functionality is administered for the treatment of the genetic disorder. In some embodiments, the composition is administered once for the treatment of the genetic disorder. In some embodiments, the composition is administered more than once for the treatment of the genetic disorder. In some embodiments, a composition used in the first treatment and the composition used in the subsequent treatments comprise the same protein variants. In some embodiments, a composition used in the first treatment and the composition used in the subsequent treatments comprise different protein variants. In some embodiments, a composition used in the first treatment and the composition used in the subsequent treatments comprise a combination of same protein variants and different protein variants.

In some embodiments, a composition comprising a protein variant or an expression vector encoding the protein variant described herein can be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In some embodiments, the first treatment comprises administering a protein variant or a protein comprising a combination of amino acid substitution mutations to the subject. In one aspect, the subsequent treatments comprise, administering the same protein variant or the protein comprising the same combination of amino acid substitution mutations to the subject as the first treatment. In other embodiments, the subsequent treatments comprise, administering a different protein variant or the protein comprising a different combination of amino acid substitution mutations to the subject as the first treatment.

In some embodiments, the composition comprising the protein variant is administered to a subject having a genetic disorder in an amount effective to correct the genetic disorder or a symptom thereof in the subject.

In some embodiments, one or more expression vectors encoding a protein variant according to any of the embodiments is administered to a subject having a genetic disorder in an amount effective to correct the genetic disorder or a symptom thereof in the subject.

In some embodiments, a variant sequence that is administered to a subject having a genetic disorder differs from the wild-type (WT) sequence of SEQ ID NO: 1 by 1 by substitution of 1 amino acid, 2, 3, 4, 5-10, 10-20, 20-30 or 30-40, 40-50, 50-60, 60-70, or 70-80 amino acids. In some embodiments, a variant sequence can comprise the wild type sequence of FIG. 1 and SEQ ID NO: 1 with 1 residue, 2, 3, 4, 5, 6, 7, 8, 9, 10, or more residues substituted. For example, 5, 10, 15, up to 20, up to 30 or up to 40, up to 50, up to 60, up to 70, up to 80 residues can be substituted. In some embodiments, one variant sequence comprising up to 5, 10, 15, 20, 30 or up amino acid substitutions can be administered. In other embodiments, a combination of variant sequences of SEQ ID NOS: 1408-1537 can be administered. In some embodiments, any one or more of the mutations listed in Table 2 can be combined, including 2, 3, 4, 5, 6, 7, 8, 9, 10, 15, 20, 25, 30, 35, 40, 45, 50, 60, 70, 80, 90, 100, or more of the amino acid substitution mutations listed in these tables. In some embodiments, any one or more of the loss of function mutants in Table 3 can be combined.

In some embodiments, the subject is a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human. In some embodiments, the subject is a human. In other embodiments, non-human mammals, especially mammals that are conventionally used as models for demonstrating therapeutic efficacy in humans (e.g. murine, primate, porcine, canine, or rabbit animals) is employed. In some embodiments, the subject is a fetus, neonate, juvenile or adult. In some embodiments, the subject has a total lack of PSAT1 enzyme. The PSAT1 enzyme is a pyridoxal phosphate (PLP) dependent homodimer and is the second enzyme in the serine biosynthesis pathway. The PSAT1 enzyme is highly conserved from bacteria to humans, and the human cDNA sequence is able to functionally replace its orthologous gene, SER1, in Saccharomyces cerevisiae. In some embodiments, the subject has a partial lack of PSAT1. In some embodiments, the subject is suspected of having PSAT1 deficiency (PSATD) (MIM: 610992), which is manifested by deleterious mutations in the human PSAT1 gene. A subject experiences symptoms consistent with a diagnosis of PSATD. A subject does not experience any symptoms or exhibits symptoms not consistent with PSATD. A subject can be diagnosed with PSATD based on DNA sequencing-based methods. A subject can be diagnosed with PSATD based on biochemical assays. A subject does not have PSATD that is detectable by DNA sequencing-based methods and/or biochemical assays. In some embodiments, the subject is at a higher risk of having PSATD. In some embodiments, the subject has a family history of PSATD. In some embodiments, the subject has a genetic predisposition to PSATD. In some embodiments, the subject is not at a higher risk of having PSATD.

In accordance with embodiments described herein, the composition comprising the protein variant can be administered by any suitable route of administration. Without limitation, the composition comprising the protein variant can be administered to the subject via oral administration, rectal administration, transdermal administration, intramuscular administration, intravenous administration, intranasal administration, or inhalation. In some embodiments, the composition comprising the protein variant is administered to liver cells. In other embodiments, the composition comprising the protein variant is administered into blood, spleen, or lung. In some embodiments, the composition comprising the protein variant can be administered by injection or in the form of a tablet, capsule, or a drink. In some embodiments, the composition comprising the protein variants of the present invention can also be used for gene therapy, using standard gene delivery protocols. Methods for gene delivery are known in the art. See, e.g., U.S. Pat. Nos. 5,399,346, 5,580,859, incorporated by reference herein in their entireties. In some embodiments, gene therapy is mediated by gene editing enzyme systems. Some non-limiting examples of the gene editing enzyme systems include CRISPR/Cas9 system, a zinc finger nuclease system, a transcription activator like effector nucleases (TALENs) system, a homing endonucleases (HE) system or a meganuclease system.

As described herein, administration of a composition comprising the protein variant can correct, treat, inhibit, or ameliorate PSATD (MIM: 610992), Neu-Laxova Syndrome 2 (NLS2) (MIM: 616038), or symptoms associated thereof. A few non-limiting examples of symptoms associated with PSATD are congenital microcephaly, seizures, psychomotor retardation, and spastic tetraparesis. A few non-limiting examples of symptoms associated with NLS2 (severe end of the spectrum for cases of PSATD) are characteristic facies with shortened eyelids, microcephaly, intrauterine growth restriction (IUGR), skin abnormalities (e.g., ichthyosis and hyperkeratosis), flexion deformities, limb malformations, edema of the hands and feet, abnormal gyration, hypoplasia of the corpus callosum, or neural-tube defects. The severe malformations manifested by NLS2 may lead to prenatal or early postnatal lethality. As disclosed herein, correct is used in a broad sense to refer to replacement or suppression of a protein variant associated with PSATD, including NLS2. In some embodiments, replacement of a protein variant associated with PSATD is performed by genetic therapy. In some embodiments, replacement of a protein variant associated with NLS2 is performed by genetic therapy. In some embodiments, the protein variant is replaced by a wild-type protein. In other embodiments, the protein variant is replaced by a highly functional protein variant. As disclosed herein, amelioration is used in a broad sense to refer to at least a reduction in the magnitude of a parameter, e.g., symptom, associated with the pathological condition being treated. In some embodiments, the method can completely inhibit, e.g., prevented from happening, or stopped, e.g., terminated, such that the host no longer suffers from the pathological condition, or at least one or more of the symptoms that characterize the pathological condition. In some embodiments, the method can delay or slowing of disease progression, amelioration or palliation of the disease state, and remission (whether partial or total), whether detectable or undetectable.

When an “effective amount” is indicated, the precise amount of the compositions of the present invention to be administered can be determined by a physician with consideration of individual differences in age, gender, weight, extent of symptoms, progression of disease, and condition of the subject.

In some embodiments, a method of enhancing the functionality of PSAT1 protein in a subject is provided. The method comprises administering to the subject the protein or composition, wherein the PSATD, NLS2, or symptom(s) thereof are reduced after administration.

In some embodiments, enhancing the functionality of PSAT1 can enhance the abundance, activity, specificity, binding affinity of PSAT1 or a combination of the aforementioned parameters. In some embodiments, the activity of the protein can comprise but not limited to enzymatic activity, non-enzymatic activity, catalytic activity, non-catalytic activity and binding activity. In some embodiments, the functionality of PSAT1 protein in the subject before administration is at least 0%, 1%, 5% 10%, 20%, 25%, 30%, 35%, 40%, 45%, 50%, 55%, 60%, 65%, 70%, 75%, 80%, 85%, 90%, 95%, 98%, 99%, or any integer that is between 1 and 99%, lower than the functionality of the wild-type protein. In some embodiments, the ratio of functionality between the wild-type protein and the protein variant before administration is lower than 0.99 (e.g., 0.98, 0.95, 0.94, 0.93, 0.92, 0.91 or lower). In some embodiments, the functionality of the PSAT1 protein is increased after administration by at least 1%, 5% 10%, 20%, 30%, 40%, 50%, 60%, 70%, 80%, 90%, 100%, 200%, 250%, 300%, 400%, or any integer that is between 1 and 400%, more than the functionality of the wild-type protein. In some embodiments, the ratio of functionality between the wild-type protein and the protein variant after administration is greater than 1.05 (e.g., 1.06, 1.10, 1.15, 1.20, or higher).

In some embodiments, the subject is a fetus, neonate, juvenile or adult.

In some embodiments, a method of administering one or more expression vectors to a subject in an amount to correct the PSATD or a symptom thereof in the subject is provided.

In some embodiments, the PSAT1 is modified to exhibit an enhanced function.

In some embodiments, the PSAT1 is replaced or supplemented by one or more polynucleotide sequences encoding SEQ ID NOS: 4-1537 to exhibit an enhanced function.

In some embodiments, the function of the protein with at least one of the amino acid substitution mutations listed in Tables 2 is enhanced by at least 5% when compared to the function of WT protein of SEQ ID NO:1.

In some embodiments, the function of the protein with at least one of the amino acid substitution mutations listed in Tables 2 is enhanced by at least 50% when compared to the function of WT protein of SEQ ID NO: 1.

In some embodiments, the function of the PSAT1 protein is enhanced by at least 5%, 10%, 15%, 20%, 25%, 30%, 40%, 50%, 60%, 70%, 80%, 90%, 100%, 200%, 300%, 400%, or any values in between 5% and 400%. In some embodiments, the functionality of PSAT1 protein in the subject before administration is at least 0%, 1%, 5% 10%, 20%, 25%, 30%, 35%, 40%, 45%, 50%, 55%, 60%, 65%, 70%, 75%, 80%, 85%, 90%, 95%, 98%, 99%, or any integer that is between 1 and 99%, lower than the functionality of the wild-type protein. In some embodiments, the ratio of functionality of the protein variant to wild-type protein before administration is less than 0.99 (e.g., 0.98, 0.95, 0.9, 0.85, 0.8, 0.75, 0.7, 0.6, or lower). In some embodiments, the functionality of the PSAT1 protein is increased after administration by at least 1%, 5% 10%, 20%, 30%, 40%, 50%, 60%, 70%, 80%, 90%, 100%, 200%, 250%, 300%, 400%, or any integer that is between 1 and 400%, more than the functionality of the wild-type protein. In some embodiments, the ratio of functionality of the protein variant to wild-type protein after administration is greater than 1.05 (e.g., 1.06, 1.10, 1.15, 1.20, or higher). In some embodiments, a subject with a mutant protein that is lower than wild-type (e.g., any of the values lower than 1.0 in the tables in the present document) and be remedied by adding a mutant protein that is higher than wild type (e.g., any of the values that are greater than 1.05 in the tables of the present document).

In some embodiments, the PSAT1 is replaced or supplemented by one or more polynucleotide sequences encoding SEQ ID NOS: 1408-1537 to exhibit an enhanced stability.

In some embodiments, the stability of the PSAT1 protein is enhanced by at least 5%, 10%, 15%, 20%, 25%, 30%, 40%, 50%, 60%, 70%, 80%, 90%, 100%, 200%, 300%, 400%, or any values in between 5% and 400%. In some embodiments, the stability of PSAT1 protein in the subject before administration is at least 0%, 1%, 5% 10%, 20%, 25%, 30%, 35%, 40%, 45%, 50%, 55%, 60%, 65%, 70%, 75%, 80%, 85%, 90%, 95%, or any integer that is between 1 and 95%, lower than the stability of the wild-type protein. In some embodiments, the ratio of stability of the protein variant to wild-type protein before administration is 0.95, 0.9, 0.85, 0.8, 0.75, 0.7, 0.6 or lower. In some embodiments, the stability of the PSAT1 protein is increased after administration by at least 1%, 5% 10%, 20%, 30%, 40%, 50%, 60%, 70%, 80%, 90%, 100%, 200%, 250%, 300%, 400%, or any integer that is between 1 and 400%, more than the stability of the wild-type protein. In some embodiments, the ratio of stability of the protein variant to wild-type protein after administration is greater than 1.05 (e.g., 1.06, 1.1, 1.15, 1.2, or higher).

In some embodiments, a kit for determining a specific mode of treatment of a genetic disorder is provided. The kit comprises a look up table. The look up table indicates specific amino acid substitutions that are commonly involved in a cellular mechanism; and one or more vectors comprising sequences of SEQ ID NOS: 4-1537 is provided.

In some embodiments, the look up table comprising a list of mutations as listed in Tables 2 and 3 is provided. In some embodiments, the table contains mutations that confer reduced activity and/or stability to the protein (Table 3 or those in Table 1 with lower than 0.99 ratio). In some embodiments, the table contains mutations that confer increased activity and/or stability to the protein (those in Table 2 or those in Table 1 with greater than 1.05 ratio).

In some embodiments, the lookup table lists a panel comprising a plurality of protein variants and their effect on functionality of protein. In some embodiments, the lookup table can be referred to select an amino acid substitution that reduces, alleviates, or limits the symptoms associated with the genetic disorder. In other embodiments, the look up table can be referred to provide functional information to select alternative functional amino acid substitutions when reverting a pathogenic amino acid substitution mutation back to the reference amino acid is not possible. A region of the protein can be selected where the frequency of amino acid substitution is significantly greater in subjects with the genetic disorder than found in a reference population without the disorder. In some embodiments, the look up table can be referred to limit a region of protein for gene therapy.

In some embodiments, the genetic disorder is PSATD.

In some embodiments, a method of identifying a subject having a genetic disorder is provided. The method comprises detecting a presence of at least one mutation in the protein that is different in the subject with the genetic disorder as compared to a subject without the disorder, wherein the presence of at least one mutation indicates the presence of the genetic disorder in the subject. These mutations can be those listed in Table 3 and/or SEQ ID NOs 4-1407.

In some embodiments, the presence of at least one mutation can be used to indicate the presence of the genetic disorder in high-risk patient population earlier than is possible for existing methods of genetic disorder detection. In some embodiments, the presence of at least one mutation can be used to indicate the presence of genetic disorder in subjects who are not at risk of developing the genetic disorder. In some embodiments, the presence of at least one mutation in the subject indicates that the subject is genetically pre-disposed to developing the genetic disorder. In some embodiments, the presence of at least one mutation in the subject indicates that the subject is at the early stages of developing the genetic disorder. In some embodiments, the presence of at least one mutation in the subject indicates that the subject is at an advanced stage of genetic disorder.

In some embodiments, the ratio of functionality between the wild-type protein and the protein variant with at least one mutation that is detected in the subject is less than 1.0. In some embodiments, the ratio of functionality of the protein variant to wild-type protein with at least one mutation that is detected in the subject is less than 0.99 (e.g., 0.98, 0.95, 0.9, 0.85, 0.8, 0.75, 0.7, 0.6, or lower) as listed in Table 3. In some embodiments, the ratio of functionality of the protein variant to wild-type protein with at least one mutation that is detected in the subject is less than 0.99 (e.g., 0.98, 0.95, 0.9, 0.85, 0.8, 0.75, 0.7, 0.6, or lower) as listed in Table 1 for those having growth estimates less than 0.99 (e.g., 0.98, 0.95, 0.9, 0.85, 0.8, 0.75, 0.7, 0.6, or lower).

In some embodiments, one or more regions of the protein can comprise 1, 2, 3, 4, 5, 6, 7, 8, 9, 10, 20, 30, 40, 50, 60, 70, 80, or more of amino acid substitutions. Many of these amino acid substitutions can be clinically actionable, such that an observed anomaly can be indicative of a genetic disorder. In some embodiments, the anomaly can be a decreased function of a protein as compared to reference subjects without the disorder. In some embodiments, the anomaly can be a decreased stability of a protein as compared to reference subjects without the disorder.

In some embodiments, a method of improving, ameliorating, or treating a genetic disorder is provided. The method comprises detecting a presence of at least one mutation in the protein that is different in the subject with the genetic disorder as compared to a subject without the disorder, wherein the presence of at least one mutation indicates the presence of the genetic disorder in the subject. The method also comprises administering to the subject at least one protein variant or composition provided herein in the embodiments, wherein the genetic disorder or a symptom thereof is reduced after administration.

In some embodiments, the mutation to be detected is selected from a group consisting of: C245R, T242M, G206R, N89Y, G119E, G13R, L40R, I25K, G78V, L63S, G78D, Y148N, G79R, S147Y, 125R, P12L, G203R, I121K, G78A, V39D, H44Y, C152F, T156M, S179L, E155Q, S84R, G11R, D176H, G79W, A99D, G78R, C152R, G119R, D176A, G105V, K200N, Y151D, D176Y, S108P, G197D, Q82R, R45S, G78C, A111P, V86D, N154Y, G13S, Q199P, Y148H, R342Q, G159R, K200Q, Y148D, K200T, E155V, A115D, L182R, A111D, Y101D, G79E, K200R, G197R, C152Y, D176N, E155K, A198D, K200M, R97G, V188D, P14T, P12R, N89K, S38R, Y101N, P87R, G13C, L255P, T104P, N154D, A109P, L90P, Q82P, N302Y, P14L, P14H, Q199K, T156P, T156R, C224W, R61P, S178P, C152W, A112P, T242P, L357R, L90R, H335D, N302K, W107R, L345R, Y289D, S108L, Y148S, Y101S, K200E, L255R, V174D, G78S, M251R, N201Y, S147F, W107C, G203D, T242R, V102E, S299R, Q199L, G203V, T242K, S344Y, R213P, D176E, S299I, R342W, Y289S, V174F, W107L, S147P, L332W, S344F, M361R, Y70D, R342P, R306P, I278N, Y150N, P243R, C291F, R342L, V212D, G105A, T156A, H158P, W107S, Y148C, M361K, R45M, V157E, C175W, Y289N, R342G, V193E, Y346N, G340R, A99P, H44L, E155G, V202D, S38I, I194N, Y70N, S178F, V102G, S178Y, R45G, V72G, N89I, Y101C, Y289H, R45K, N241I, R45T, Y70S, Y346S, I91S, V103G, F10S, G334W, H335L, R213G, I278S, C175R, V149E, Y151N, N89D, I282N, N302H, C291R, Q199E, S179P, G340D, I307N, A115T, 191T, P243H, V72E, S183P, V149G, L332S, S285Y, Q76R, D353V, F305S, A198P, A99T, A324P, H44N, P244R, S272P, M329R, E155A, H44P, G11V, Y346C, L64P, V174G, G119V, C175F, G105E, N154I, T156S, S275P, L270P, A111V, G340C, F181S, S247R, H335P, E155D, H44Q, F191S, I303N, T104K, G119W, Q276P, P14A, L59P, N122I, L17R, A115V, P14S, G11W, S299C, L22W, F364L, R45W, N241Y, Y70C, L75R, N201H, G334V, I121R, P87H, Y230D, V124D, L216R, D187Y, P87L, S179A, G170V, Y289C, A358P, V293M, S178T, G287V, P67L, A205D, W140L, N154T, P292R, V293E, G339V, S275Y, M361T, Y150C, V60E, A343D, W140R, S204Y, F364S, V193G, F83Y, V86G, H335R, A99V, P292Q, Y151S, L321P, R213L, I211S, N89T, E161K, F320C, N180K, S147T, F10I, G263V, P292A, C245Y, I282S, P166L, F364C, F288C, G340S, P166T, I282T, P292S, I303F, E161V, I278T, A111T, G197V, A153P, P166H, C245F, R213C, L64R, M361V, H335Q, T104R, H335N, S344P, S285C, P166S, L216P, F320I, W140G, V338E, G206V, V66F, L228P, F364V, F195S, S43R, Q233P, W140C, Y279H, V188G, R61W, R298I, A146D, D283H, L63W, I303S, M42R, C224Y, S285T, V209E, F320L, F364I, V174L, L357Q, R298S, I307S, V103E, C175Y, E317D, R298T, A106T, 1341N, L216Q, N180Y, E317G, T139P, V172G, D163V, A112E, S285A, A324D, E366V, I282F, H158Y, F181C, Y279D, A359P, F191V, N347Y, I281F, S275T, K274I, P12T, Y279F, I303M, G92V, P292L, P166A, R213S, V293A, M177K, V186G, R298K, D163Y, N328I, I134L, V210E, V60G, R298G, V202G, Y279S, D353A, G206E, V21E, I134T, N89S, A264D, C80R, T277P, D100Y, F49S, Q233R, F320Y, F162V, G197S, G262V, Y230S, I134N, A15D, K94I, H368R, Y70H, I134S, I307T, D353Y, A358D, E114K, V290G, V160M, V160G, F118S, Q286P, S204P, V124G, T56R, H158R, M329I, N347D, E161D, F288S, F162I, I248S, K333N, N180H, L182Q, R61L, F10V, T104I, A15P, F288V, K33I, W257S, K274E, M267K, N302D, I281N, F191L, G79A, N347T, D100G, I211T, F305L, F83C, E366K, I303T, L182P, C291S, V186E, V293L, M329V, L316S, I281S, F162S, D163H, D100V, F288I, M267R, A348G, D283V, A99G, S204F, P67R, R5G, P225L, Y230H, L17M, V124F, F162C, V102L, I134F, I278F, G77E, V21G, V160A, V209G, F288L, F360S, C224R, F181L, Y230C, N89H, S189F, I282M, F191C, D314A, L75V, F10C, S178C, S20L, T56P, P67A, R306G, N69I, I258T, I134M, K190E, E57K, S179T, P126A, F305Y, H158L, H335Y, I341L, F305V, E317K, T277R, F83I, P87S, P244L, F83V, F49C, D187G, D283G, L316V, P4L, D100A, K190N, A15V, K356R, A220D, E280K, S108T, Y32D, G263R, V354G, S84T, M361L, I307F, V250F, P225H, F74S, E268V, E114A, E366A, S299T, N284I, A234P, Y101H, D353G, V168D, G218W, P67T, A106P, C152G, P18Q, I194S, C98F, D68Y, A99S, L90F, V202A, N54I, V103M, V290L, P67Q, Y151C, F49L, N58K, Y230N, L327P, V338G, G203C, V102A, T242A, R61Q, F49V, V354A, P135L, K190T, I194F, A109V, H125D, D163A, S46P, Y101F, S337P, K51Q, L59V, N297T, V124A, I307M, A266P, E280V, G159A, C224F, V149A, K116R, N154H, G263D, I52M, Y240D, Y346H, E352V, E229D, F181V, V86F, G197A, D187V, A106G, N54T, F162L, F181I, K333Q, D136A, P67S, D136G, G339E, D68A, W257C, G262A, K274R, S84I, I248N, G159C, K71N, M42V, V349D, I282L, N328Y, L228R, Q76E, V212G, S84C, R222G, E326Q, V35F, S43I, V7G, S183T, Q76H, K190R, V66L, S130R, Q276E, F305I, L40I, G287R, T104S, A343G, D167V, G235V, A220V, V254D, G77V, V157G, R213H, L239V, S238P, D187H, N302S, S275C, G339A, L217M, A196T, A220P, T208I, D100E, S247C, A264G, E62V, E161Q, V290D, S183C, Q6H, D167Y, H19L, I165L, S299G, S20P, Y32F, C98W, C175S, A153T, I258S, E161G, P137R, N284Y, L216M, D187A, A343S, Y279N, K51R, I123F, V21A, S130T, G36C, G308D, V188F, P126R, D215V, K33R, V207F, D100H, S183Y, N328S, I91V, K127R, L217R, I91F, N143K, N201K, V21L, N297D, G262R, G312V, P135T, N69D, N55I, V60A, N69Y, V250D, I165R, K300T, N201I, K323N, I53N, S20T, Q276L, K190Q, L128V, S204C, D187N, F83L, D215N, D353N, H19Q, G119A, E57D, G334E, A265T, Y32H, I73S, S108A, V35I, G81S, V186M, I165K, S331T, L23I, E317Q, V39L, T139S, P126L, H19D, P18A, V212F, G81C, H158Q, L173P, A348V, L316F, N180I, E57V, G235E, P304A, D145V, E24D, A112T, K323R, Q296P, S43G, I351V, N69K, G334R, R222P, T120S, E28A, E366G, V168I, V72A, D353H, I341S, L93S, L142H, T139N, D31E, P137T, F181Y, N309D, V103A, L75P, A85V, H125P, R222L, D163E, D215H, K333E, G129W, N309I, Q369L, G170A, R306C, S247I, V103L, P18R, N347H, E57G, I25M, P244S, W257G, L325H, I273S, I123V, K300E, I53S, Q82L, L327H, L270R, G36V, A266V, V21M, I282V, K300M, S271G, S189P, A50G, C224S, V124L, I52L, T277A, E62Q, K323Q, D313Y, A171S, N328K, I52T, I165M, K27Q, K16R, E24G, F288Y, L255Q, A220T, F195C, T132R, Q286R, G92A, V188A, Q369R, H125L, E256Q, T56A, E268K, P137Q, N54D, L59I, E57A, S147A, K94E, K51M, S237N, V60L, L253M, N236H, G235A, I211F, K16Q, A65V, D283A, D163G, G287A, P225A, K127N, K33E, F364Y, L59H, V39F, A109G, A171V, V227D, T277I, L217Q, M177T, H19P, F164S, G263C, E223V, L216V, D48N, L217V, E229Q, P295L, E113V, F10L, F164I, I303V, S38G, K323E, N58T, N297H, M267T, RST, V186A, N58I, K27T, A146P, G308A, K133I, P244T, G252V, C224G, G92S, G81V, V202L, V168L, V290I, T104A, P185A, Y240H, E28Q, L345M, G36R, A198V, S272T, K333I, I341M, G263S, E280G, K274T, I165T, D2A, Q296H, A109D, E114V, Q355H, K318E, V254I, D322V, A153S, C98Y, D215Y, I73N, N241T, N141H, K363N, V35L, T120P, I281V, P225S, V212I, A109S, D68G, K274Q, L221P, D215G, L30F, E24V, L64V, D100N, M177V, L128F, G92C, V160L, A198G, E28D, P87T, S147C, E268A, R5M, V290A, A85T, P225T, S247G, R319G, H19R, L365W, I307V, L142V, E223K, L128R, S183A, T132I, W257R, G312R, P135A, D313A, Q276K, P87A, S204A, S38C, K362R, V207L, V250I, S331Y, T139A, A3D, A65D, E294G, H368P, A146G, S237I, T139I, F164Y, N54Y, K184N, R61G, V102M, V60M, I307L, I52N, K363R, D187E, E317A, V149L, E62A, S130C, A265S, D145E, V202I, N122H, Q82E, L64Q, M267L, L59F, S130G, I248F, D145A, K16E, S237G, D167E, Y150H, Q286L, N297Y, A95E, K300N, S247T, K33Q, I123T, K269E, M251T, A153G, Y230F, P135R, F164V, A171P, D145Y, N328T, L270V, I25V, I121L, S189C, A266S, V35G, C98S, M301T, V212L, V7M, D167N, I121M, T350I, P295T, A358V, K190M, P126S, Q276H, G287E, A3S, D215E, K127T, N58Y, F164C, Q296E, F118Y, L142I, L40V, G218A, G96V, M267V, N284D, L88I, D167H, V8D, P185L, P244A, T277S, A310V, R5K, L330M, F164L, L217P, A343P, N141S, N58H, D31V, G312E, L270F, S237C, E268G, A324S, F74V, D314Y, D2V, L370I, N55S, I25L, E366D, L345P, A265P, L17V, A315P, K333R, K363E, V254L, Q369P, V86A, T120I, V254A, D48V, Y70F, E268Q, N261T, N309T, P244Q, K269M, G92D, C152S, N141Y, S238Y, G218R, N9S, V232L, I258L, N284S, E256D, S84N, Q369H, A146S, K231E, V254G, H368D, L253W, E256V, G262E, R306S, Q233K, I278M, E294V, V174A, N261I, S43C, K71R, S138C, L325P, D322G, D2G, A358T, A106V, K269N, V232E, E114G, F360I, P295A, L253V, V210G, V124I, I123M, V207G, I278V, K318I, F246S, E113D, A3T, K27M, S46L, L327R, G218E, L88S, A65G, Q286E, Q296K, K184R, P18S, Q6R, F360Y, D48A, A146V, 1273L, L332V, N141D, V168A, V232G, I53V, L59R, T277K, N328D, Q233H, S272C, I52S, L75Q, S130N, L327F, N141K, D322N, S138I, D314H, Y151H, L142R, K274N, G96A, L40F, A234S, K184E, N58S, L182V, E294K, M301K, P12H, L327V, K362T, I91L, G81A, I73T, I351S, K362Q, L40H, L330V, Q26L, N309Y, V212A, A266D, G308S, F360C, A111G, D68H, S247N, A50D, S331P, K363Q, E326A, K362N, K33N, E229V, Y249D, N261K, K27N, G170R, K311N, A220S, P4A, I278L, A112V, I273N, T132P, L221F, S237T, D214H, L142P, G218V, G36D, I341F, E268D, L142F, G308R, A315S, D31N, A264P, R306L, S47A, G34V, I211V, I258N, E223Q, N284H, A50S, P295S, Q6E, A358G, L321H, D214A, A205S, V188I, Y240F, I273M, F118L, D313E, D314G, G36A, I273V, N122T, R319I, A65S, C291G, G34R, F219Y, S271R, Q276R, M42L, G339R, E57Q, V168G, P18L, K169N, D322E, A310P, E113A, V66I, K259I, S331A, N260H, L29S, I123L, I52F, S337T, M367R, L90V, K231Q, F219V, K127Q, D214G, P126H, A324T, I194T, Y249S, Q76P, I37S, D145N, G77A, D314E, E294Q, K127E, D313G, S138N, A324G, D48H, Y240C, D68N, G129A, I281L, V202F, L365S, D322A, E280D, I273F, K110R, K27E, E161A, N201S, F219L, V193A, L239W, K363T, K269T, I248V, N143D, D31G, Q355L, E280Q, A196V, L93W, W257L, K323T, Q82H, V250L, S272Y, G252D, D313H, P304R, L270H, Q6P, A264S, I211M, L128I, N309K, V86I, N122D, N55Y, M367V, H19N, V172E, H368N, K184T, K16T, D136N, R97K, A266T, R336T, N260I, N260Y, D2N, S272F, I351L, M329L, L321V, A343V, G129E, N54K listed in Table 3 is provided.

In some embodiments, the mutants provided herein are mutants that result in protein variants of reduced activity and/or stability.

In some embodiments, the mutants are classified as mutants that result in protein variants of reduced activity and/or stability if the ratio of activity of the protein variant to wild-type protein is less than 0.99 (e.g., 0.98, 0.95, 0.9, 0.85, 0.8, 0.75, 0.7, 0.6 or lower).

In some embodiments, the mutants are classified as mutants that result in protein variants of reduced activity and/or stability if the ratio of activity of the protein variant to wild-type protein is less than 0.99 (e.g., 0.98, 0.95, 0.9, 0.85, 0.8, 0.75, 0.7, 0.6, or lower).

In some embodiments, the functionality of the mutants that result in protein variants of reduced activity and/or stability is at least 0%, 1%, 5% 10%, 20%, 25%, 30%, 35%, 40%, 45%, 50%, 55%, 60%, 65%, 70%, 75%, 80%, 85%, 90%, 95%, 98%, 99%, or any integer that is between 1 and 99%, less than the functionality of the wild-type protein. In some embodiments, the ratio of functionality of the protein variant to wild-type protein is less than 1.0. In some embodiments, the ratio of functionality of the protein variant of reduced activity and/or stability to wild-type protein is less than 0.99 (e.g., 0.98, 0.95, 0.9, 0.85, 0.8, 0.75, 0.7, 0.6 or lower) as listed in Table 3.

In some embodiments, the activity of the mutants that result in protein variants of reduced activity and/or stability is at least 0%, 1%, 5% 10%, 20%, 25%, 30%, 35%, 40%, 45%, 50%, 55%, 60%, 65%, 70%, 75%, 80%, 85%, 90%, 95%, 98%, 99%, or any integer that is between 1 and 99%, less than the activity of the wild-type protein. In some embodiments, the ratio of activity of the protein variant to wild-type protein is 0.99 or less than 1.00. In some embodiments, the ratio of activity of the protein variant of reduced activity and/or stability to wild-type protein is less than 0.99 (e.g., 0.98, 0.95, 0.9, 0.85, 0.8, 0.75, 0.7, 0.6, or lower). In some embodiments, the activity of the protein can comprise but not limited to enzymatic activity, non-enzymatic activity, catalytic activity, non-catalytic activity and binding activity.

In some embodiments, the expression of the mutants that result in protein variants of reduced activity and/or stability is at least 0%, 1%, 5% 10%, 20%, 25%, 30%, 35%, 40%, 45%, 50%, 55%, 60%, 65%, 70%, 75%, 80%, 85%, 90%, 95%, 98%, 99%, or any integer that is between 1 and 99%, less than the expression of the wild-type protein. In some embodiments, the ratio of expression of the protein variant to wild-type protein is 0.99 or less than 1.00. In some embodiments, the ratio of expression of the protein variant of reduced activity and/or stability to wild-type protein is less than 0.99 (e.g., 0.98, 0.95, 0.9, 0.85, 0.8, 0.75, 0.7, 0.6, or lower).

In some embodiments, the abundance of the mutants that result in protein variants of reduced activity and/or stability is at least 0%, 1%, 5% 10%, 20%, 25%, 30%, 35%, 40%, 45%, 50%, 55%, 60%, 65%, 70%, 75%, 80%, 85%, 90%, 95%, 98%, 99% or any integer that is between 1 and 99%, less than the abundance of the wild-type protein. In some embodiments, the ratio of abundance of the protein variant to wild-type protein is 0.98 or less than 0.99. In some embodiments, the ratio of abundance of the protein variant of reduced activity and/or stability to wild-type protein is less than 0.99 (e.g., 0.98, 0.95, 0.9, 0.85, 0.8, 0.75, 0.7, 0.6, or lower).

In some embodiments, the specificity of the mutants that result in protein variants of reduced activity and/or stability is at least 0%, 1%, 5% 10%, 20%, 25%, 30%, 35%, 40%, 45%, 50%, 55%, 60%, 65%, 70%, 75%, 80%, 85%, 90%, 95%, 98%, 99%, or any integer that is between 1 and 99%, less than the specificity of the wild-type protein. In some embodiments, the ratio of specificity of the protein variant to wild-type protein is less than 0.99. In some embodiments, the ratio of specificity of the protein variant of reduced activity and/or stability to wild-type protein is less than 0.99 (e.g., 0.98, 0.95, 0.9, 0.85, 0.8, 0.75, 0.7, 0.6, or lower).

In some embodiments, the affinity of the mutants that result in protein variants of reduced activity and/or stability is at least 0%, 1%, 5% 10%, 20%, 25%, 30%, 35%, 40%, 45%, 50%, 55%, 60%, 65%, 70%, 75%, 80%, 85%, 90%, 95%, 98%, 99% or any integer that is between 1 and 99%, less than the affinity of the wild-type protein. In some embodiments, the ratio of affinity of the protein variant to wild-type protein is less than 1.00. In some embodiments, the ratio of affinity of the protein variant of reduced activity and/or stability to wild-type protein is less than 0.99 (e.g., 0.98, 0.95, 0.9, 0.85, 0.8, 0.75, 0.7, 0.6 or lower).

In some embodiments, mutants that result in protein variants of reduced activity and/or stability exhibit a change in functionality, abundance, activity, specificity, affinity or a combination of the aforementioned parameters as compared to the wild-type.

In some embodiments, the presence of one or more mutants that result in protein variants of reduced activity and/or stability in the subject can indicate the presence of a genetic disorder in the subject. In some embodiments, a composition comprising one or more mutants that result in protein variants of increased activity and/or stability as provided in the embodiments is administered to the subject with the genetic disorder. In some embodiments, the administration of one or more mutants that result in protein variants of increased activity and/or stability can replace one or more mutants that result in protein variants of reduced activity and/or stability in the subject. In other embodiments, the administration of one or more mutants that result in protein variants of increased activity and/or stability can suppress one or more mutants that result in protein variants of reduced activity and/or stability in the subject. In other embodiments, the administration of one or more mutants that result in protein variants of increased activity and/or stability can enhance the functionality of the mutants that result in protein variants of reduced activity and/or stability in the subject.

In some embodiments, the genetic disorder is PSATD.

### Development of an Assay for Determining High Functional Protein Variants

As provided herein, an assay was developed to determine the impact of different protein variants on the functionality and stability of PSAT1.

A construct encoding the protein variant can be used for expression in a yeast system that has been knocked out of the yeast homolog. For example, the yeast can be genetically engineered to have the gene SER1, the yeast ortholog of human PSAT1 protein, knocked out. The yeast can then be genetically engineered to express PSAT1 from a nucleic acid. Expression constructs for yeast are known by those of skill in the art and may have a strong yeast promoter/terminator and may comprise a yeast selectable marker cassette.

An assay can then be run to determine the yeast cells growth in comparison to a yeast cell expressing the wild type PSAT1 or a yeast cell expressing a PSAT1 gene that has no mutations. Loss of function or enhanced functionality of the yeast enzymes produces a quantitative and easily measured trait, poor growth in the absence of serine supplementation.

In some embodiments, the assay is performed using the steps provided herein. In some embodiments, the assay can comprise one or more of: 1) yeast cells expressing wild type PSAT1 or variant proteins are grown to saturation in rich liquid medium, 2) the yeast cells are then pinned onto solid medium utilizing glycerol as the carbon source (YPG: 1% yeast extract, 2% peptone, 2% glycerol, 2% Bacto Agar) to remove any respiratory-deficient yeast cells, 3) yeast cells are then pinned back into rich liquid medium (YPD: 1% yeast extract, 2% peptone, 2% glucose) and grown to saturation, 4) yeast cells are then pinned in replicate (n=3-6) onto solid minimal medium (SD: 1.7 g/L Bacto Yeast Nitrogen Base without amino acids or ammonium sulfate, 0.5% ammonium sulfate, 2% glucose, 2% Bacto Agar), which lacks serine, and grown for 3 days at 30 °C, and 5) pinning is performed using a Biomek i7 robot outfitted with a V&P 96-pin head. In some embodiments, the assay can be used for any relative comparison regarding functionality as provided herein, including the claims.

In some embodiments, functional comparison of different protein variants disclosed herein with the WT protein is made using the assay provided herein.

### Kits

Also provided herein are kits including the expression constructs, and protein sequences provided and described herein as well as written instructions for making and using the same. Thus, for example, provided herein is a kit comprising one or more of: a protein sequence as described herein; and/or an expression construct as described herein.

A look up table is also provided for use with the kit to assess the combination of mutations that indicate enhanced functionality or stability. The look-up table is a table that correlates one form of data to another form, or one or more forms of data to a predicted outcome to which the data is relevant, such as phenotype or trait. For example, a look-up table can comprise a correlation between allelic data for at least one protein that is involved in a metabolic pathway and a particular trait or phenotype, such as a particular disease diagnosis, that an individual who comprises the particular allelic data is likely to display, or is more likely to display than individuals who do not comprise the particular allelic data. Look-up tables can be multidimensional, for example, without being limiting, they can contain information about multiple alleles for a particular protein or proteins simultaneously, or they can contain information about multiple protein variants, such as mutational variants, and they can also comprise other factors, such as particulars about diseases diagnoses, therapeutic methods or drugs to be used with a particular disease. In some embodiments, the look-up table comprises correlations between at least one allele and a disease.

A look-up table can be generated by the individual assessment of protein variants or by studies of cells that have the mutated form of the protein that is to be tested.

Development of a Dietary Treatment after Detection of Protein Variants

Also provided herein is a method of improving, ameliorating, or treating a genetic disorder, the method including: 1) receiving diagnostic information, the diagnostic information comprising a detection of a presence of a DNA variant that results in at least one mutation in a protein that is different in the subject with genetic disorder as compared to a subject without the disorder, wherein the presence of at least one mutation indicates the presence of genetic disorder in the subject; and 2) administrating to the subject a treatment, wherein the genetic disorder or a symptom thereof is reduced after administration. In some embodiments, the treatment comprises an effective amount of a dietary supplementation. In some embodiments, the dietary supplementation comprises L-serine. In some embodiments, the dietary supplementation comprises glycine. In some embodiments, the dietary supplementation comprises L-serine and glycine. In some embodiments, the effective amount of L-serine comprises 100 mg/kg/day, 300 mg/kg/day, 400 mg/kg/day, 500 mg/kg/day, or 950 mg/kg/day. In some embodiments, the effective amount of L-serine comprises 100-950 mg/kg/day. In some embodiments, the effective amount of L-serine comprises 300-950 mg/kg/day. In some embodiments, the effective amount of L-serine comprises 400-950 mg/kg/day. In some embodiments, the effective amount of L-serine comprises 500-950 mg/kg/day. In some embodiments, the effective amount of glycine comprises 200 mg/kg/day. In some embodiments, the effective amount of glycine comprises 400 mg/kg/day. In some embodiments, the dietary supplementation comprises L-serine and glycine. In some embodiments, the effective amount comprises 500 mg/kg/day of the L-serine and 200 mg/kg/day of the glycine. In some embodiments, the effective amount comprises 300 mg/kg/day of the L-serine and 400 mg/kg/day of the glycine. In some embodiments, the effective amount comprises: 300 to 500 mg/kg/day of the L-serine; and 200 to 400 mg/kg/day of the glycine.

In some embodiments, the genetic disorder is PSATD. In some embodiments, the genetic disorder is NLS2. In some embodiments, reduced activity of PSAT1, or the metabolic pathway in which it functions, give rise to certain diseases that include (but are not limited to) ichthyosis, epilepsy, hypertension, retinal degeneration, and macular telangiectasia type 2. While epilepsy and hypertension are common diseases, and a small fraction of cases involving these diseases are due to their PSAT1 genotype, detecting a PSAT1 mutation that confers activity below a certain disease-causing threshold may inform the availability of additional treatment options that include dietary supplementation.

In some embodiments, the subject is a fetus and the subject indirectly receives the treatment by way of a carrier of the subject. In some embodiments, the subject is a newborn. In some embodiments, the subject is an infant. In some embodiments, the subject is a child. In some embodiments, the subject is a juvenile. In some embodiments, the subject is an adult. In some embodiments, the one mutation that indicates the presence of the genetic disorder in the subject is selected from the mutations in Table 3.

In some embodiments, a method of improving, ameliorating, or treating a disorder is provided, the method comprising: receiving diagnostic information, the diagnostic information comprising a detection of a presence of a DNA variant that results in at least one mutation in a protein that is different in the subject with a disorder as compared to a subject without the disorder, wherein the presence of at least one mutation indicates the presence of the disorder in the subject; administrating to the subject a treatment, wherein the disorder or a symptom thereof is reduced after administration. In some embodiments, the disorder is associated with reduced activity in serine biosynthesis. In some embodiments, the reduced activity in serine biosynthesis is due to reduced activity of PSAT1. In some embodiments, the DNA variant comprises at least one mutated allele of the PSAT1 gene. In some embodiments, the at least one mutated allele of the PSAT1 gene is inheritable.

In some embodiments, the disorder comprises ichthyosis. In some embodiments, the disorder comprises epilepsy. In some embodiments, the disorder comprises hypertension. In some embodiments, the disorder comprises retinal degeneration. In some embodiments, the disorder comprises macular telangiectasia type 2. In some embodiments, the mutation protein comprises an amino acid substitution of at least one amino acid of PSAT1. In some embodiments, PSAT1 is on diagnostic panels for ichthyosis, epilepsy, retinal degeneration, hypertension, macular telangiectasia type 2, or any combination thereof. In some embodiments, reduced PSAT1 activity below a threshold, as informed by the diagnostic panel, indicates the disorder. In some embodiments, to improve, ameliorate, or treat any of the above disorders, the treatment comprises an effective amount of a dietary supplementation. In some embodiments, the dietary supplementation comprises L-serine. In some embodiments, the effective amount of L-serine comprises 100 mg/kg/day, 300 mg/kg/day, 400 mg/kg/day, 500 mg/kg/day, or 950 mg/kg/day. In some embodiments, the effective amount of L-serine comprises 100-950 mg/kg/day. In some embodiments, the effective amount of L-serine comprises 300-950 mg/kg/day. In some embodiments, the effective amount of L-serine comprises 400-950 mg/kg/day. In some embodiments, the effective amount of L-serine comprises 500-950 mg/kg/day. In some embodiments, the effective amount of glycine comprises 200 mg/kg/day. In some embodiments, the effective amount of glycine comprises 400 mg/kg/day. In some embodiments, the dietary supplementation comprises L-serine and glycine. In some embodiments, the effective amount comprises 500 mg/kg/day of the L-serine and 200 mg/kg/day of the glycine. In some embodiments, the effective amount comprises 300 mg/kg/day of the L-serine and 400 mg/kg/day of the glycine. In some embodiments, the effective amount comprises: 300 to 500 mg/kg/day of the L-serine; and 200 to 400 mg/kg/day of the glycine.

### Example 1. Expression Construct Encoding Protein Variants of PSAT1

The human PSAT1 sequence can functionally replace the corresponding SER1 gene in yeast. This single copy can be stably integrated into the genome under the control of the native yeast regulatory sequences for transcription, translation, and mRNA stability with the nucleic acid comprising the sequence.

The PSAT1 sequence used is based on the amino acid sequence for PSAT1 isoform 1 (GenBank: NP_478059.1) that was optimized for expression in yeast (hereafter yPSAT1, GenBank: MN654100, MN654101) (SEQ ID NO: 1538) as depicted in FIG. 6. At amino acid positions conserved between the yeast and human sequences, the codon sequence for the yeast ortholog, SER1, in the S288c reference sequence (R64.2.1 annotation in Saccharomyces Genome Database) was selected. In the non-conserved locations, codons for the appropriate PSAT1 amino acid were chosen to be as similar as possible to the usage frequency of the codons at corresponding SER1 codons. Alleles containing a frameshift mutation utilized this same sequence up to the frameshift and the remaining open reading frame (ORF) of human PSAT/isoform 1 cDNA (GenBank KJ898759.1).

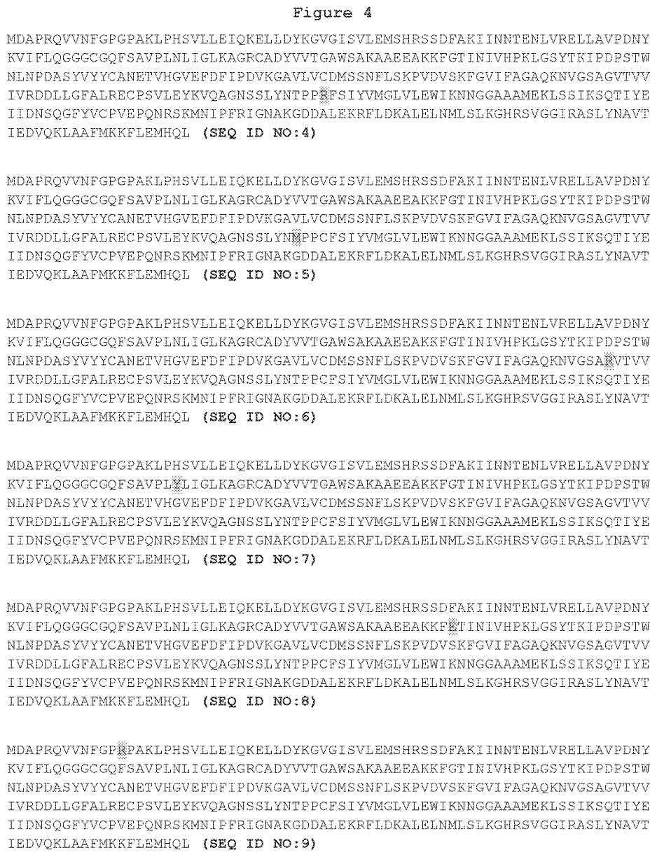

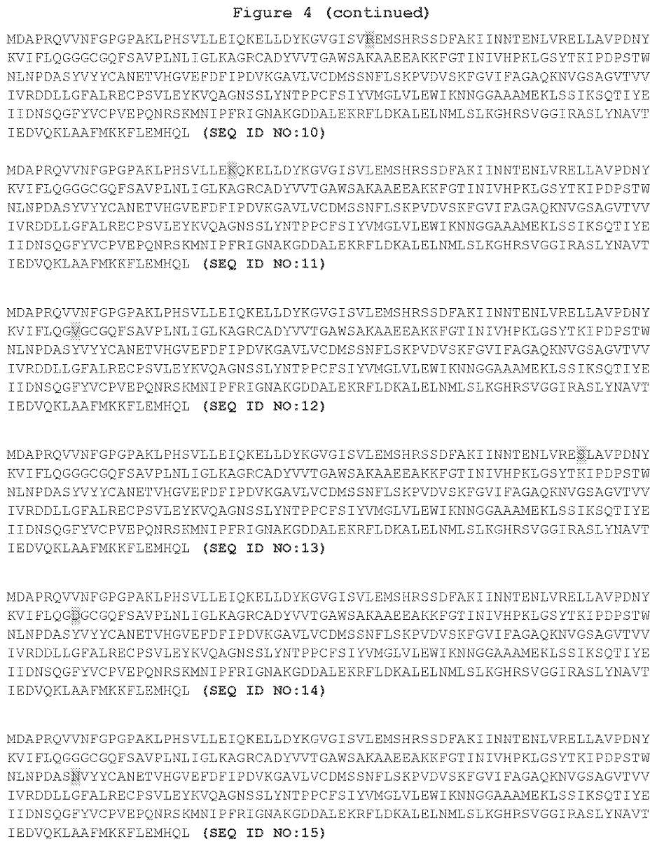

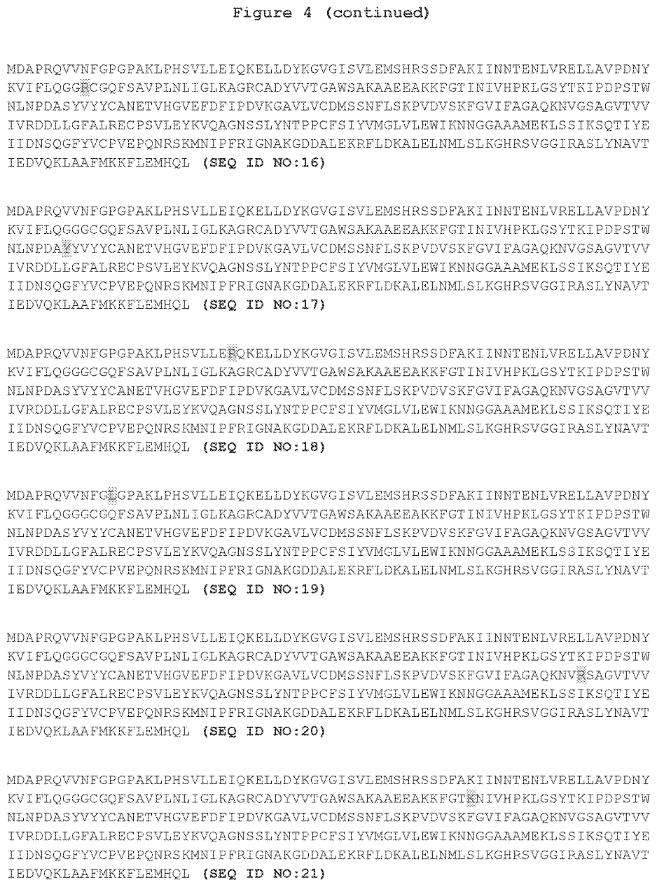

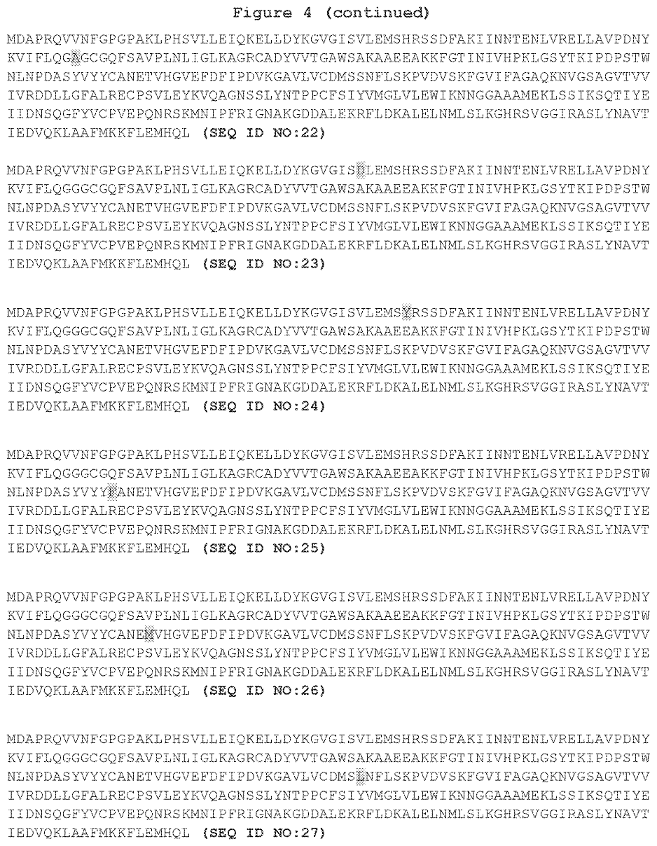

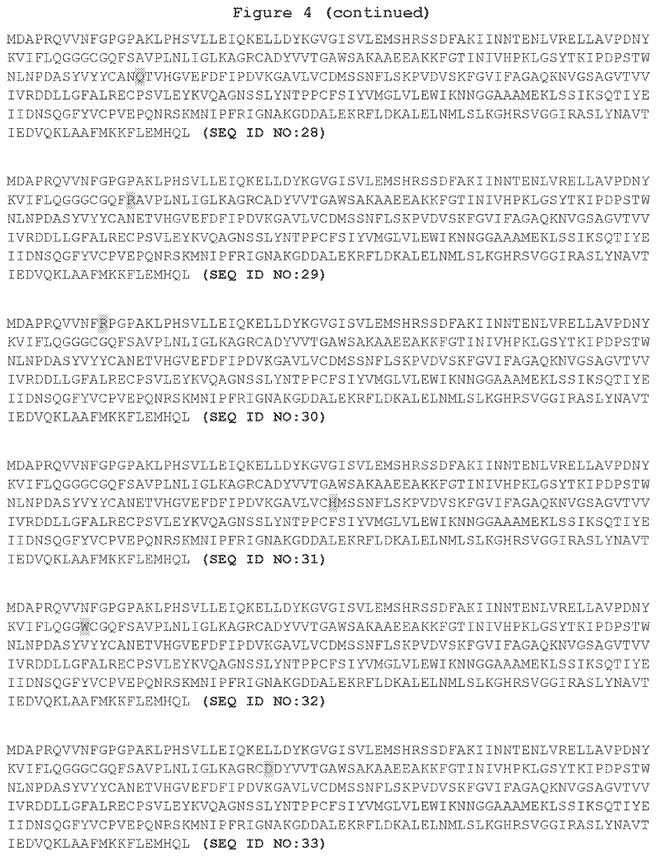

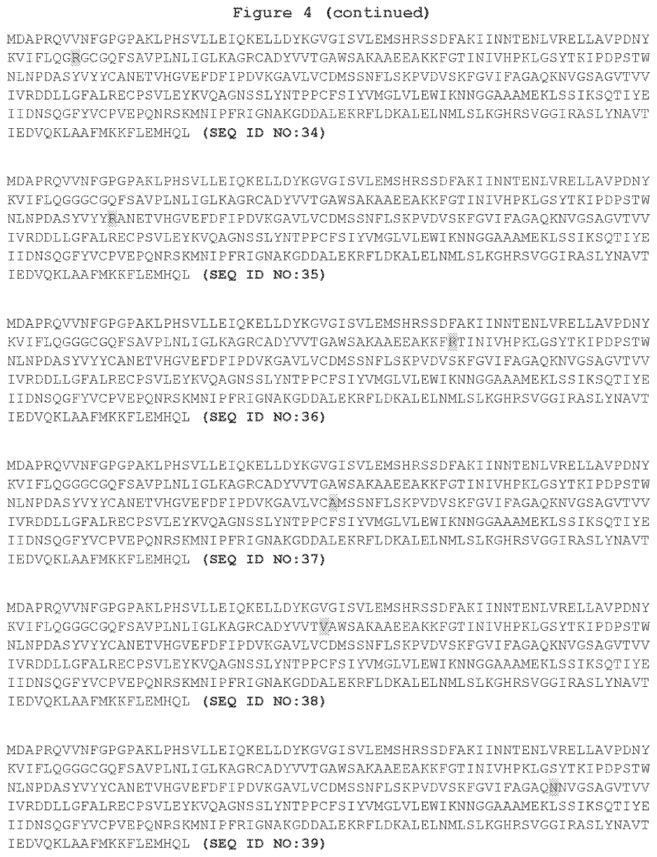

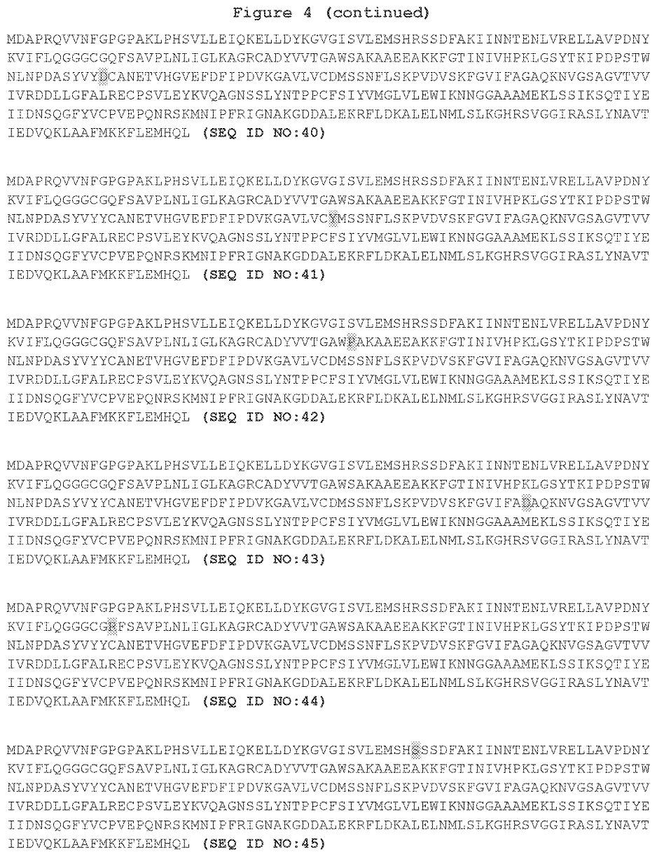

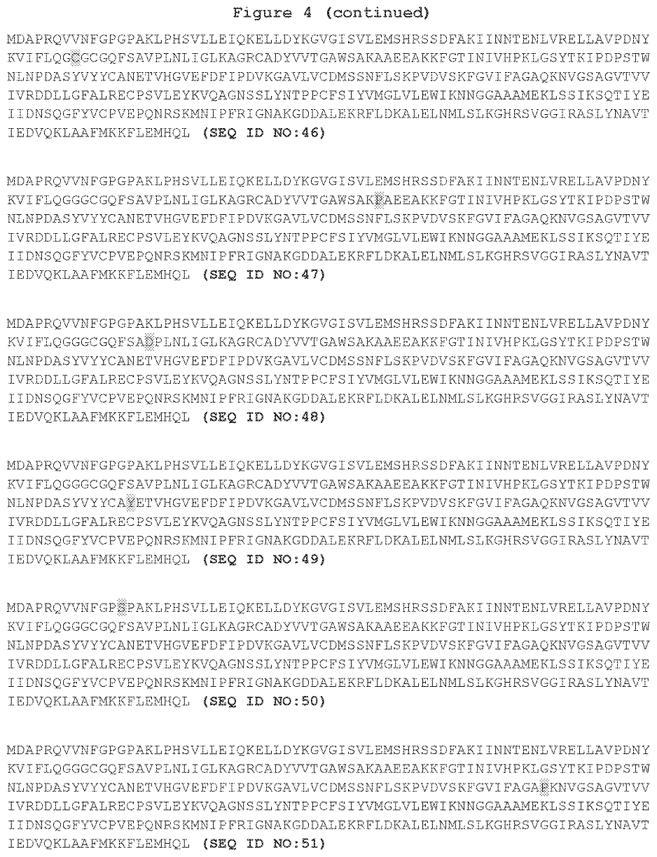

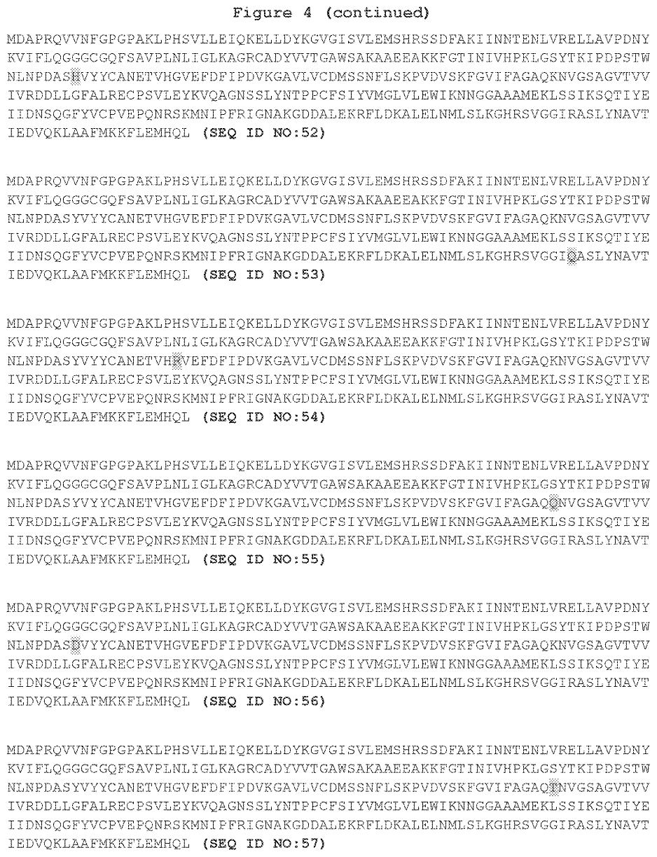

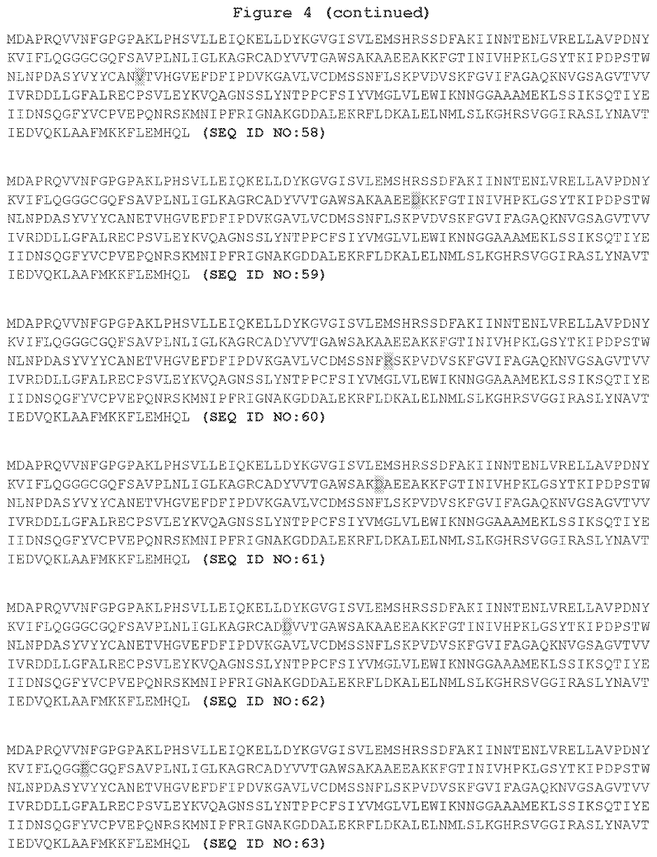

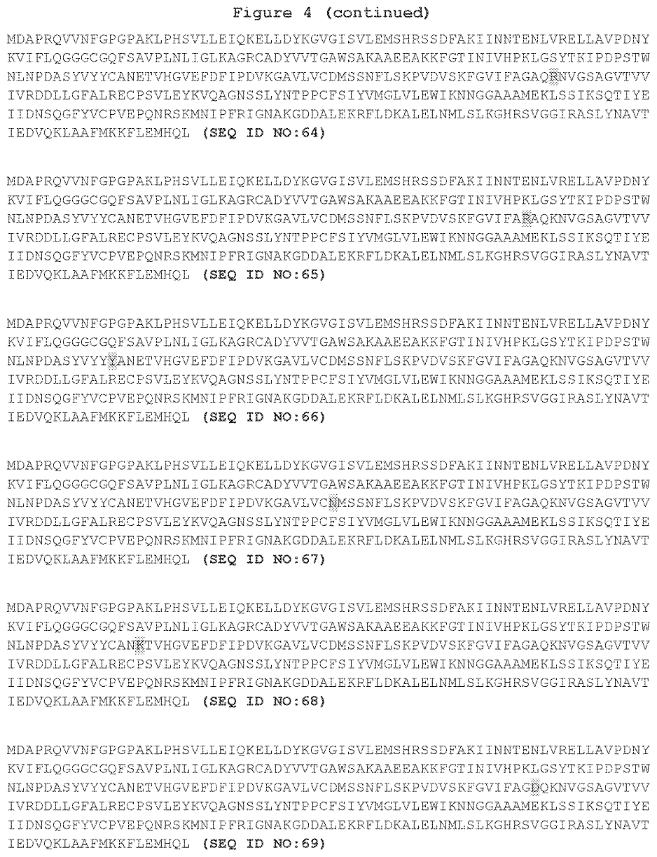

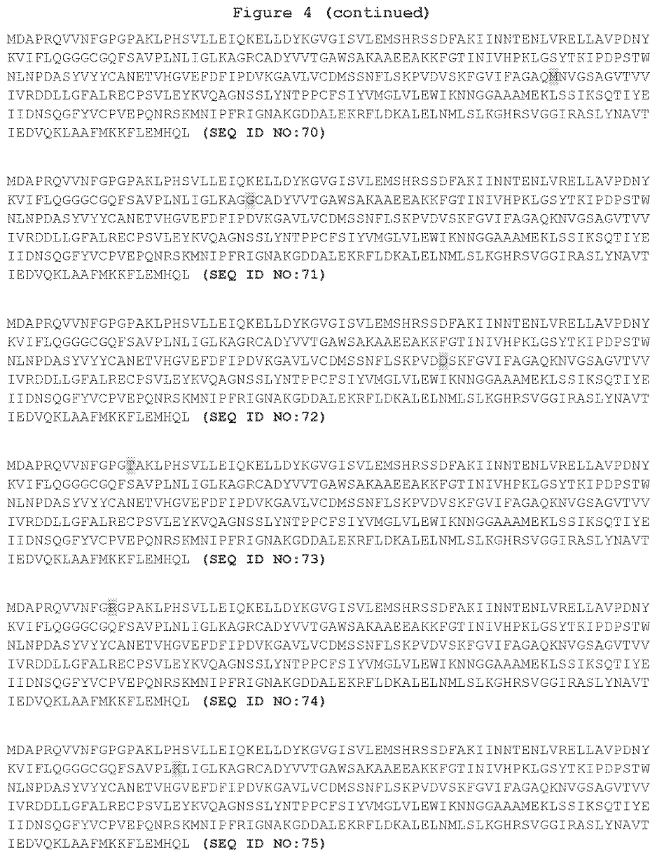

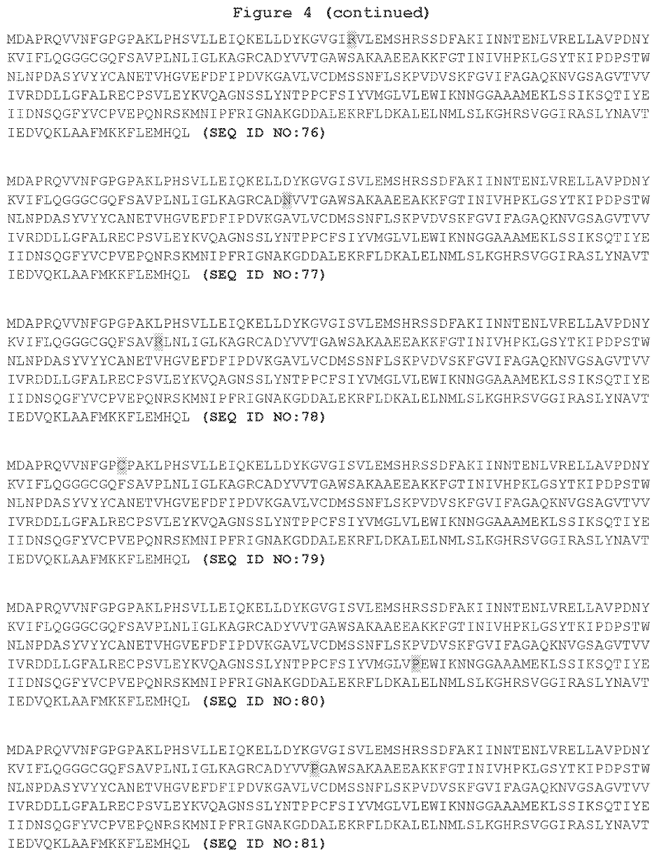

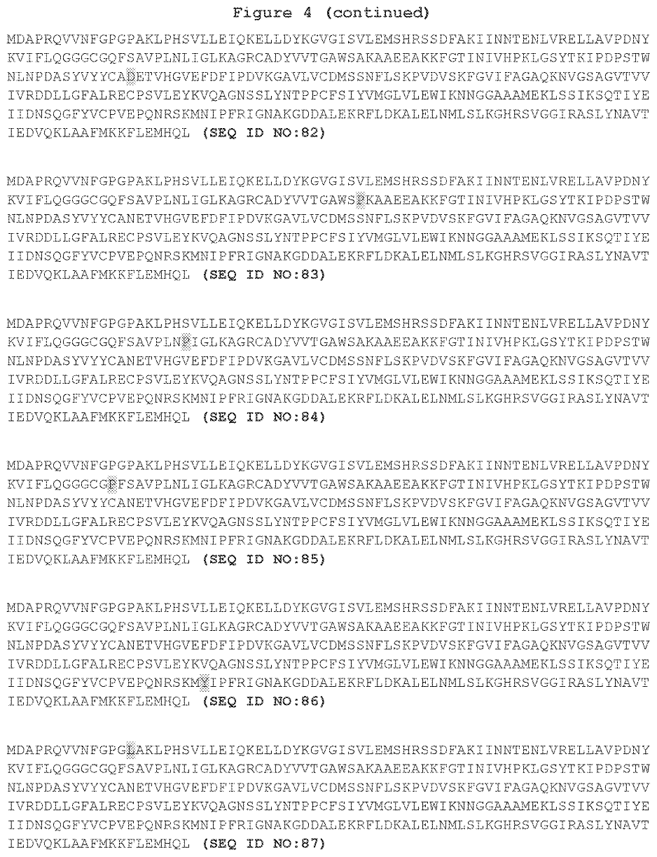

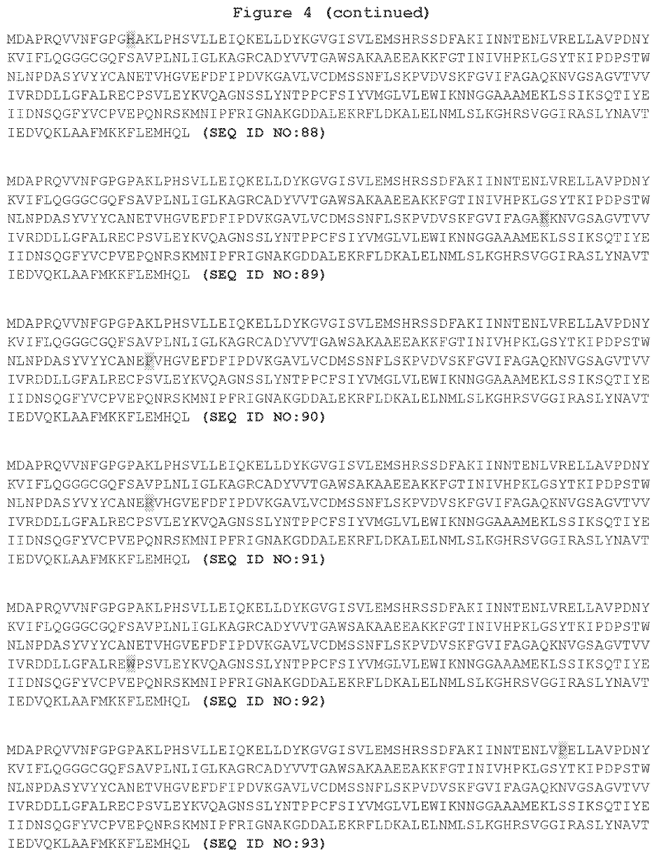

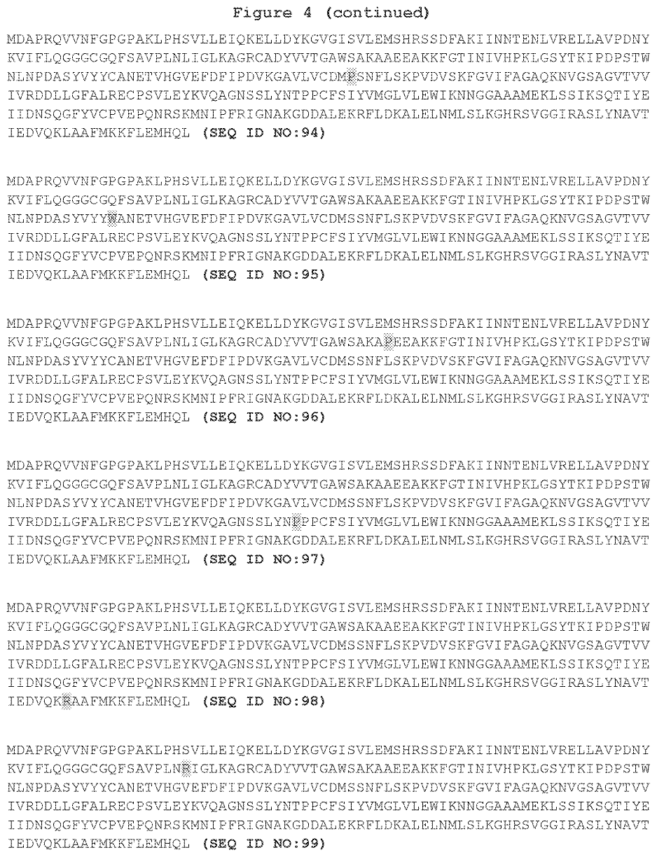

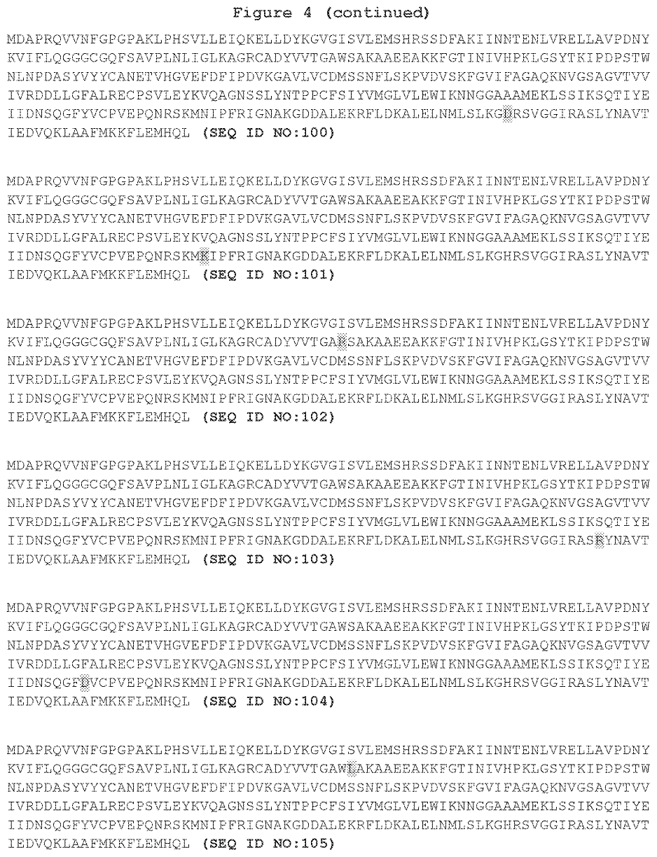

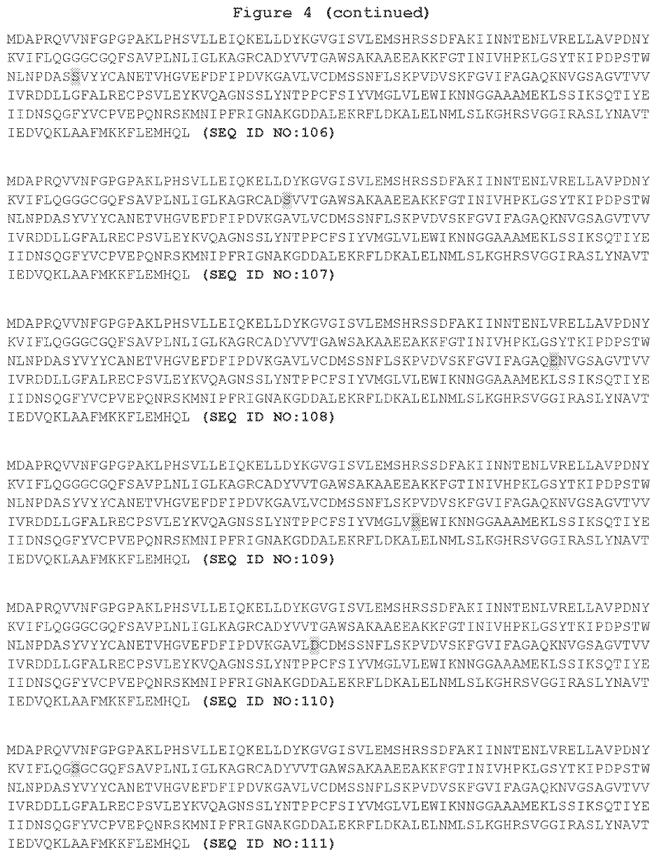

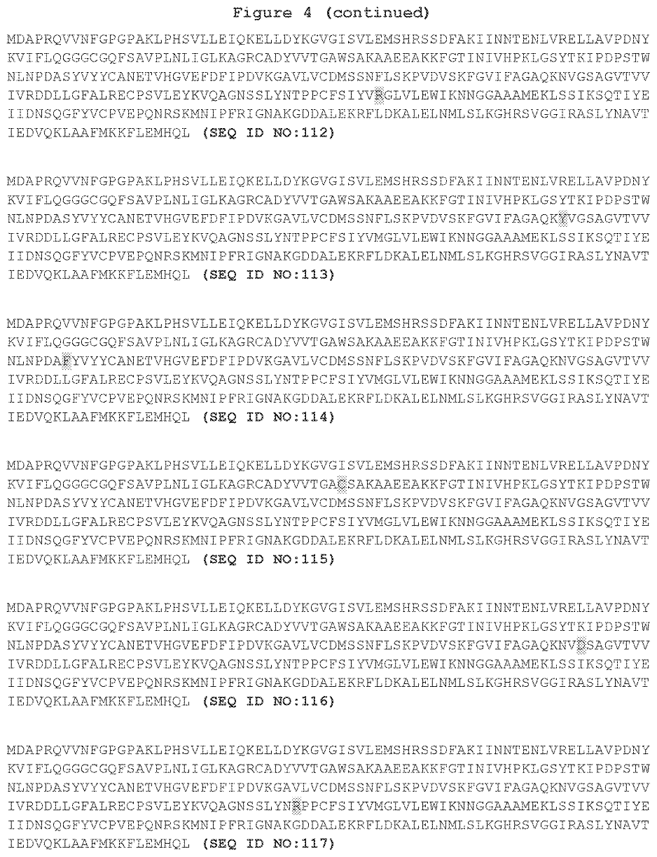

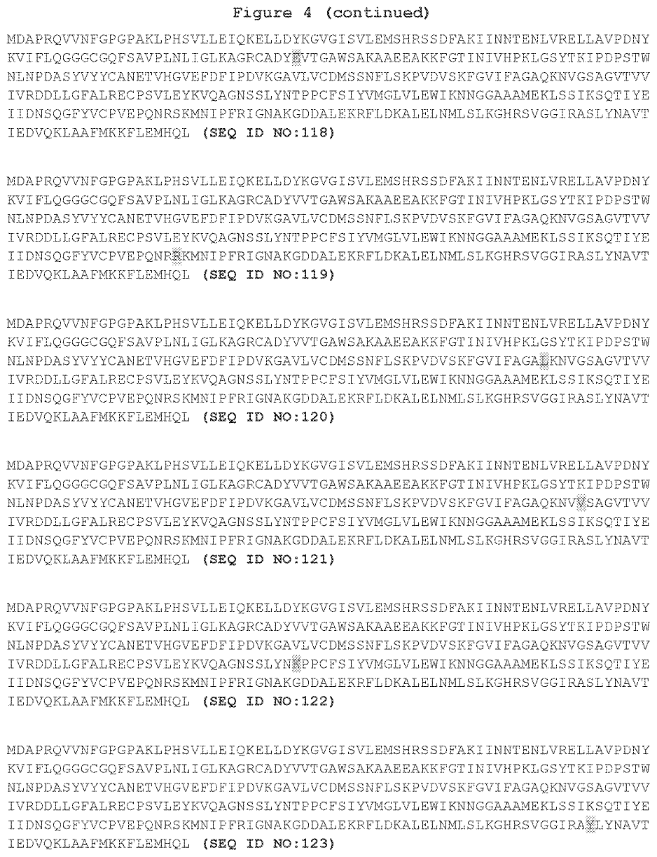

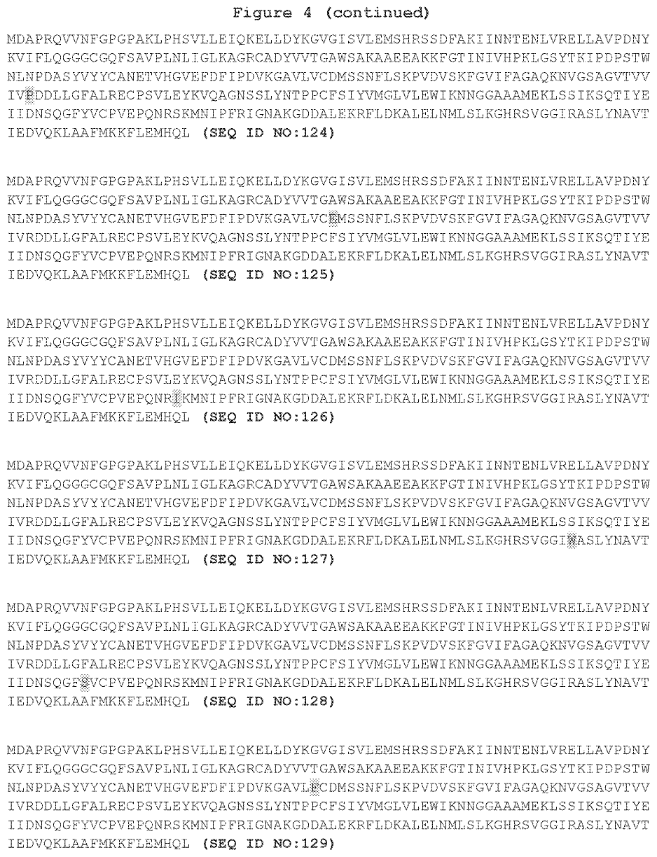

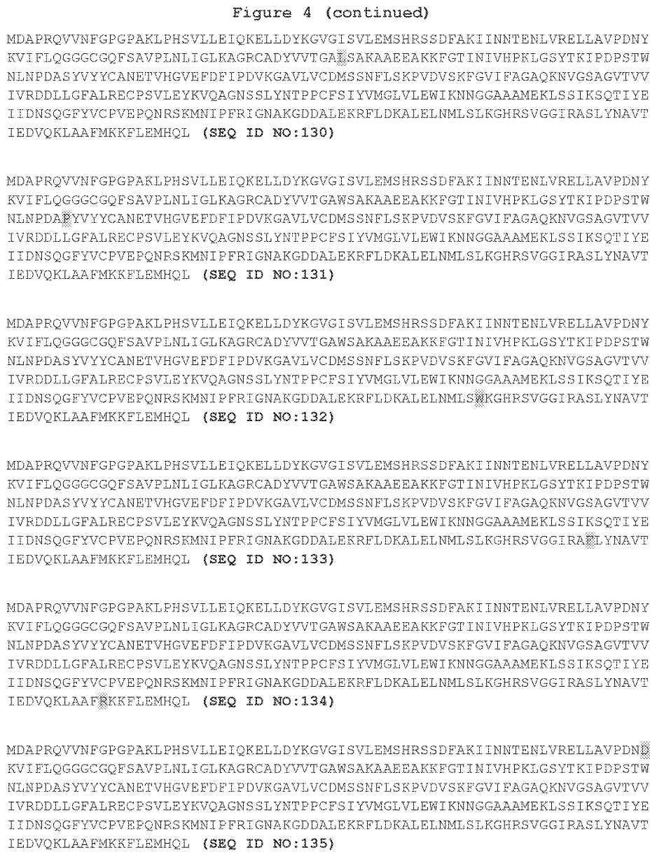

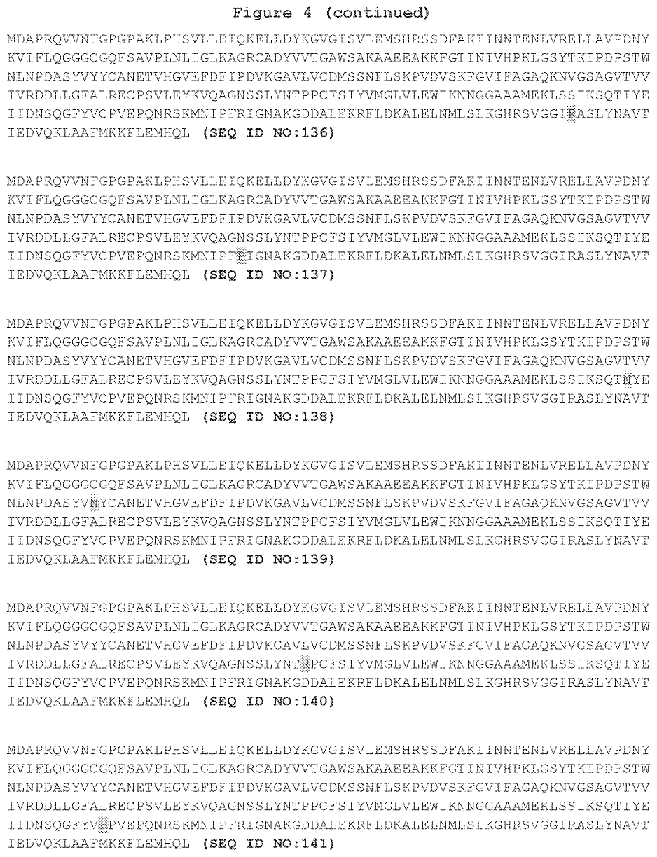

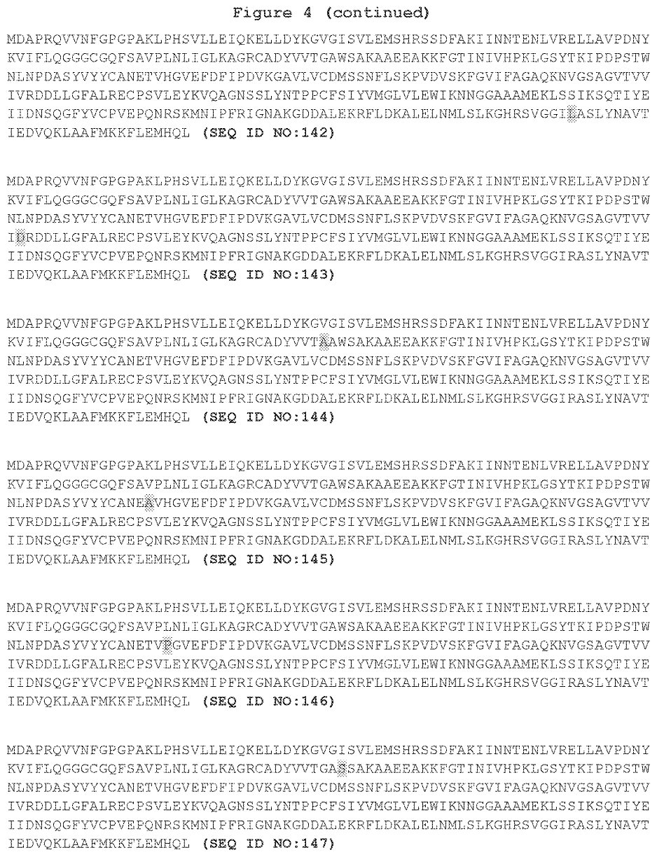

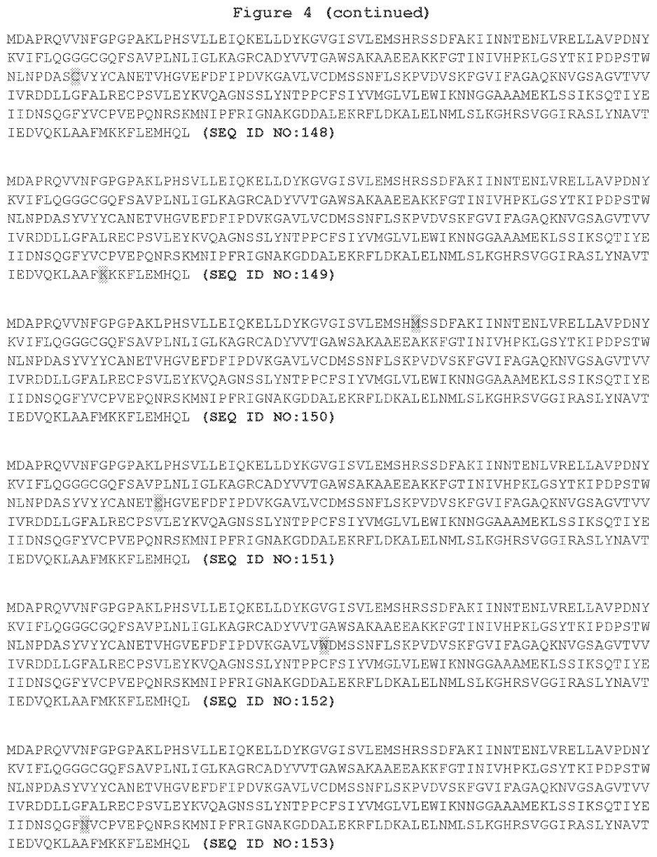

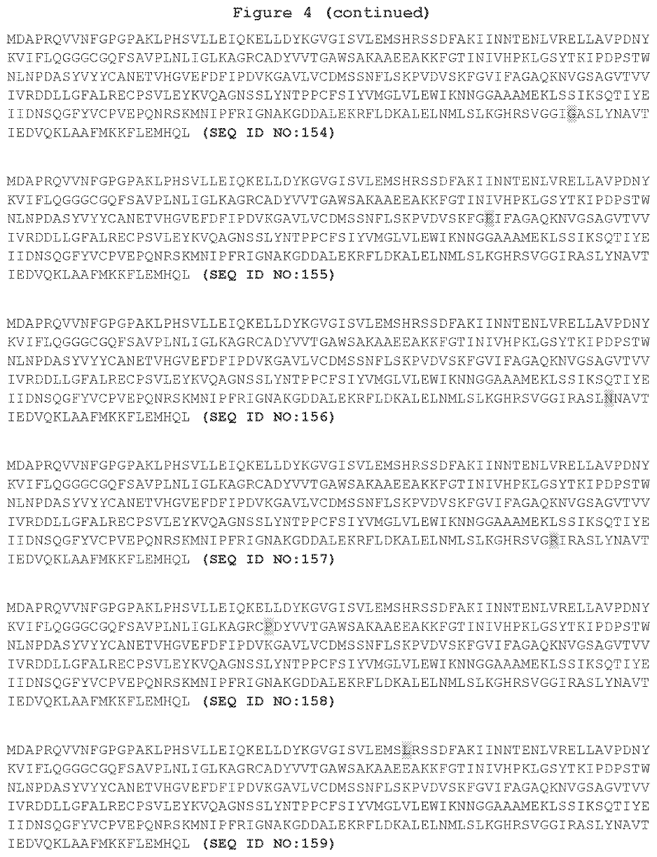

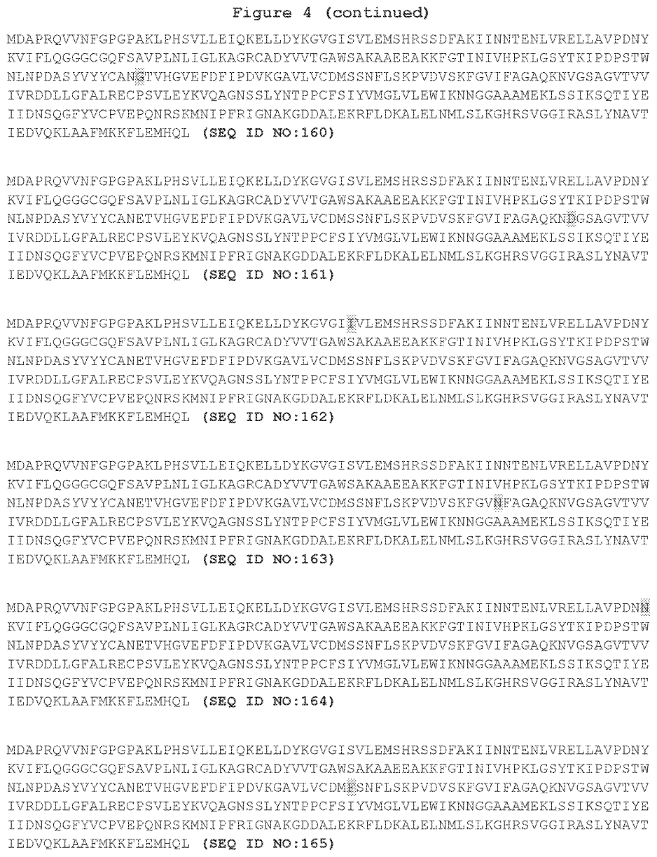

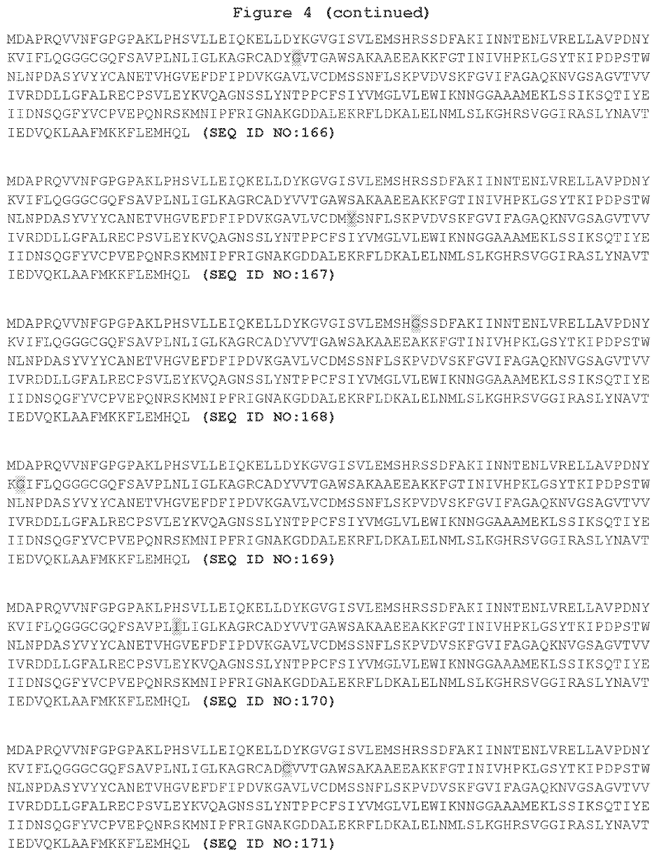

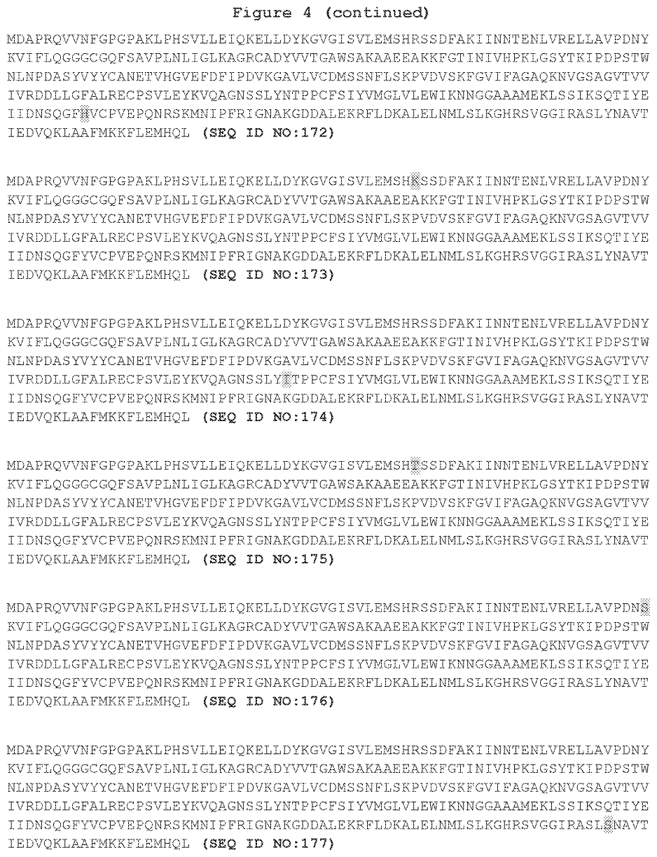

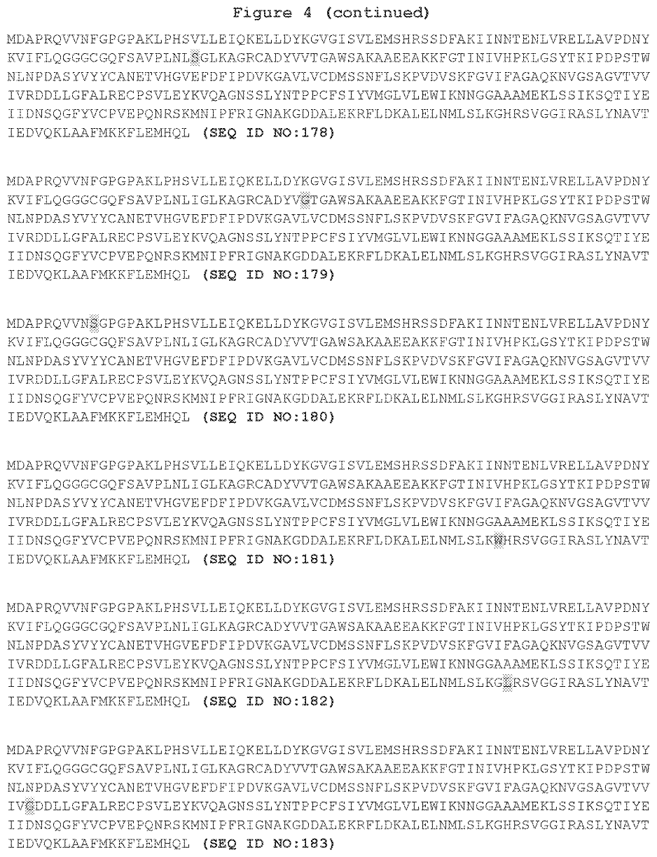

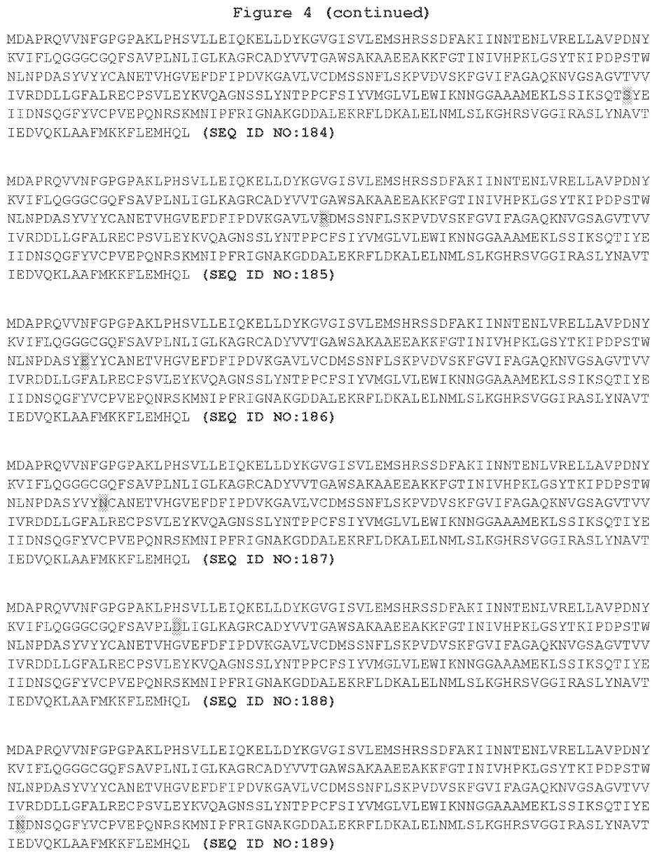

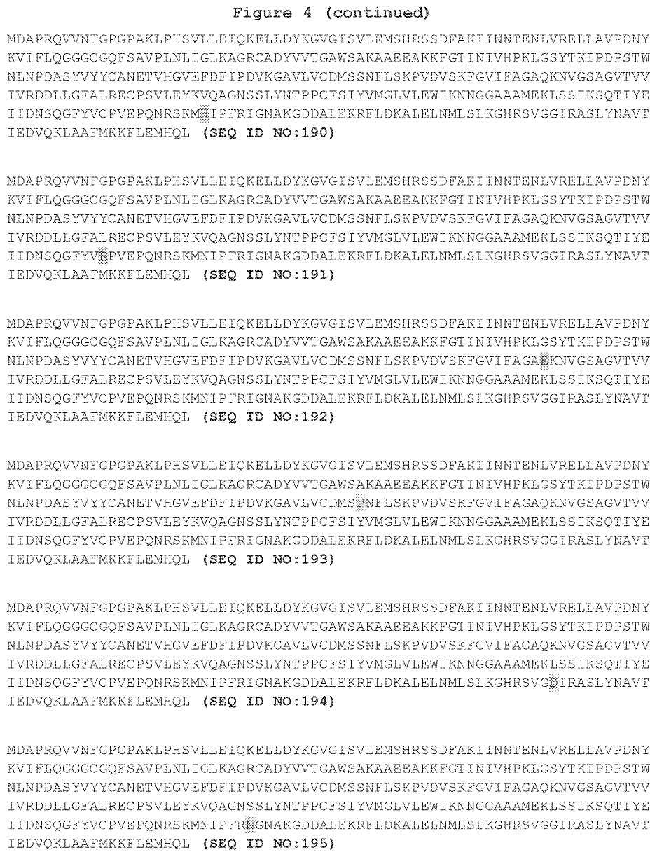

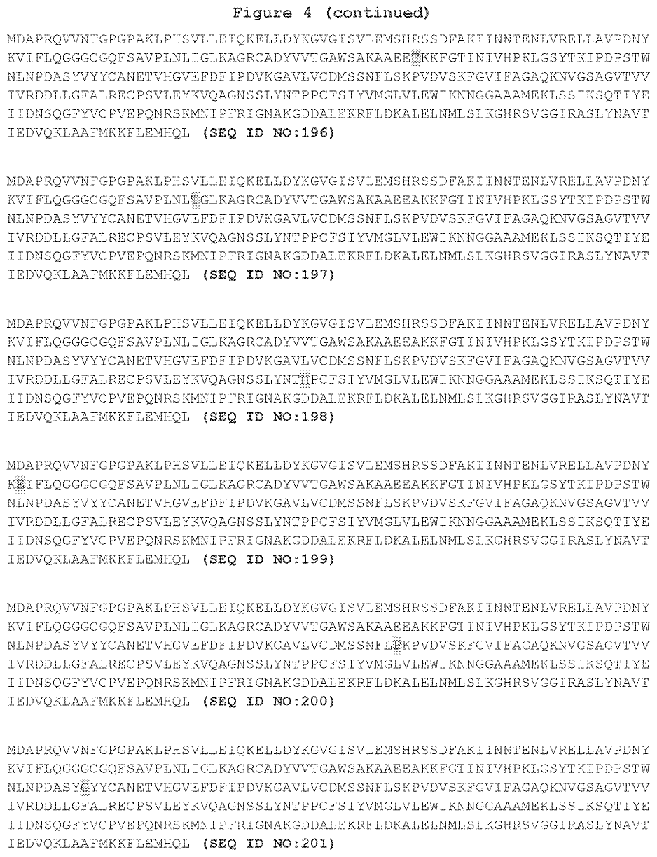

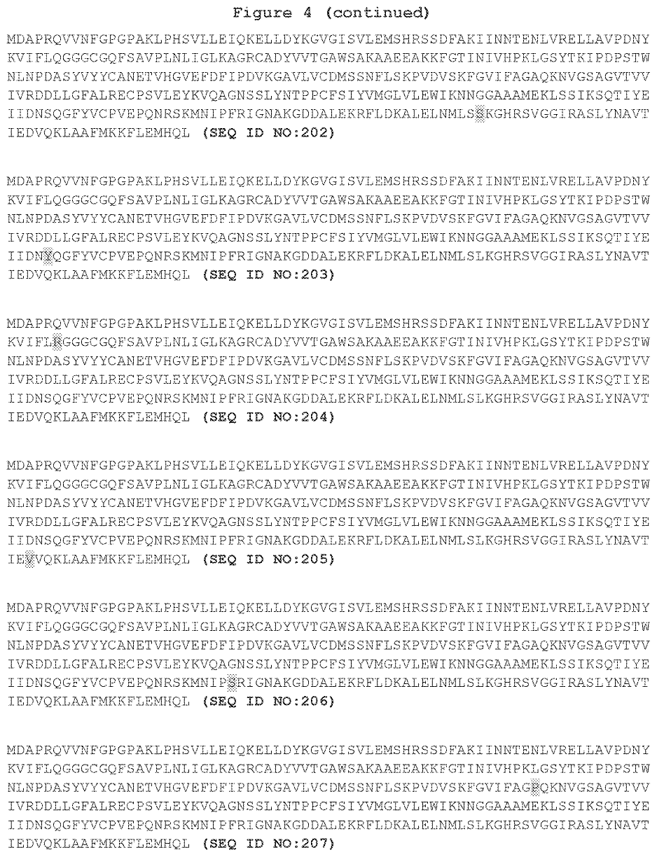

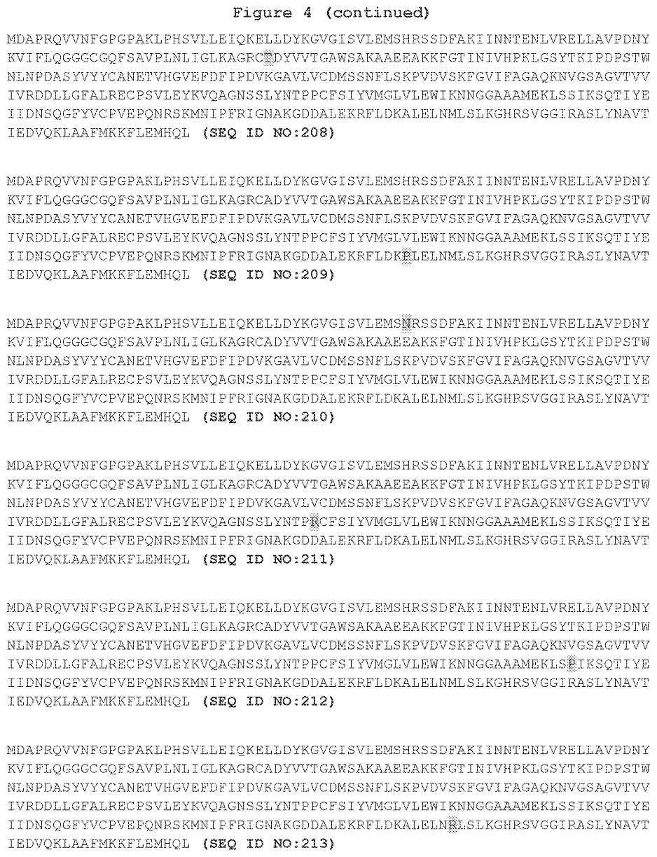

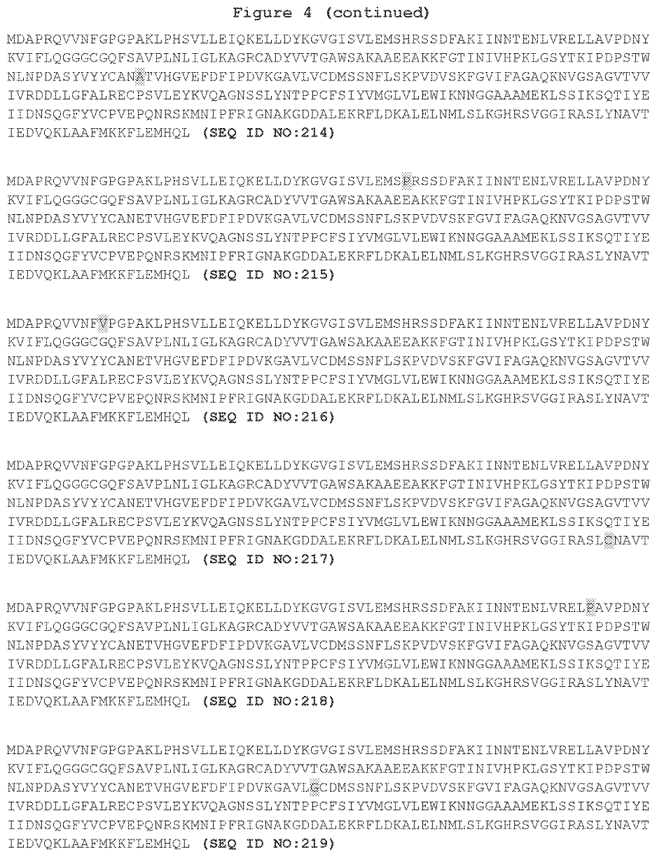

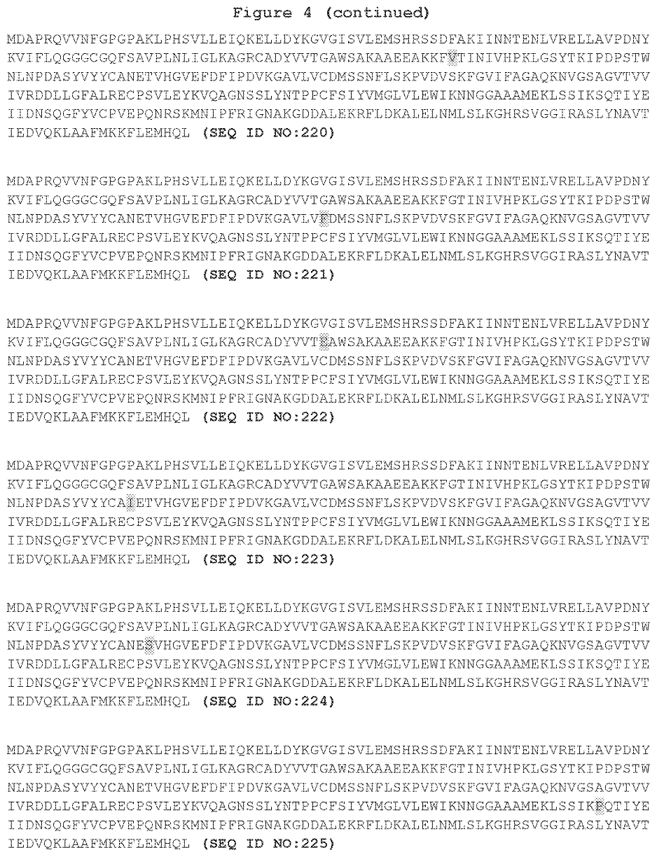

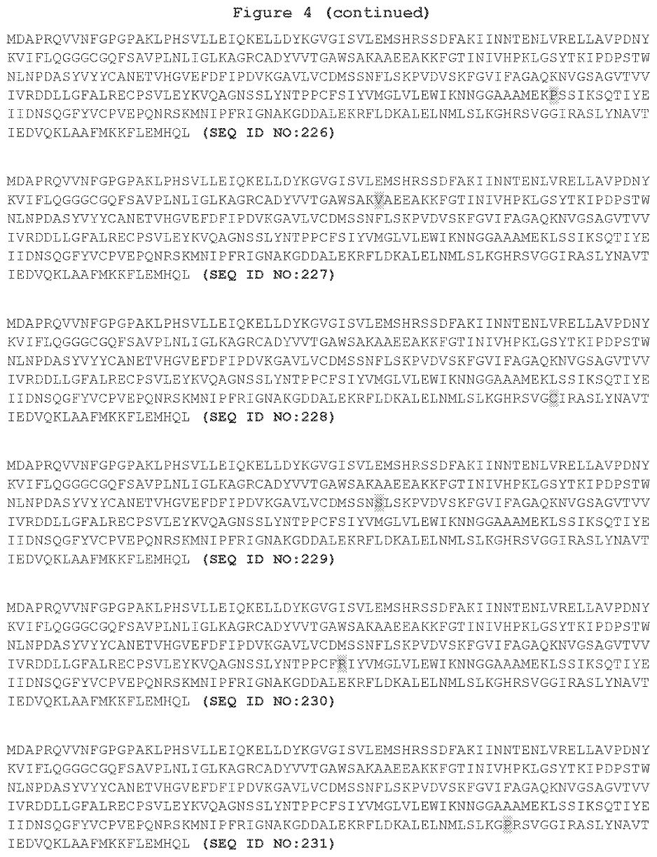

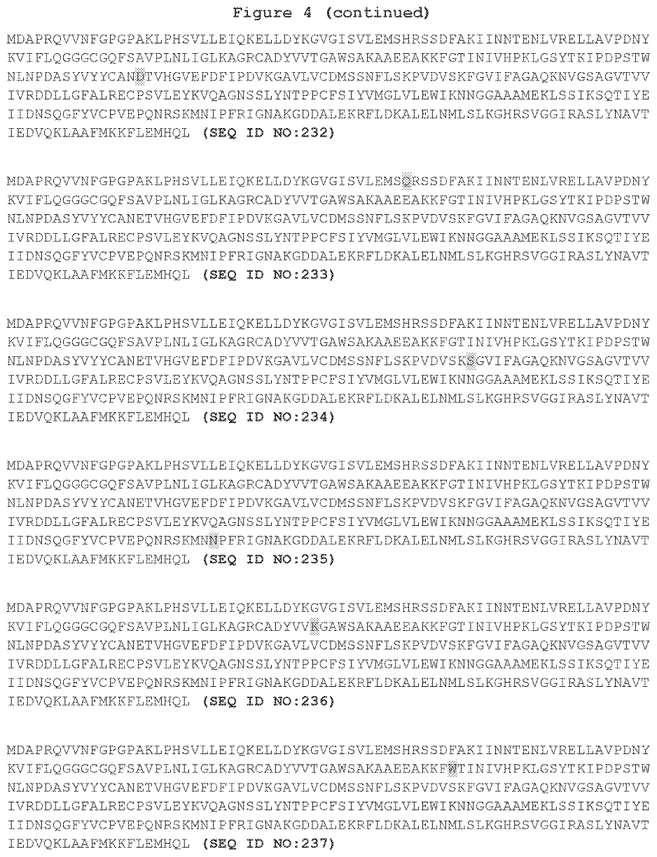

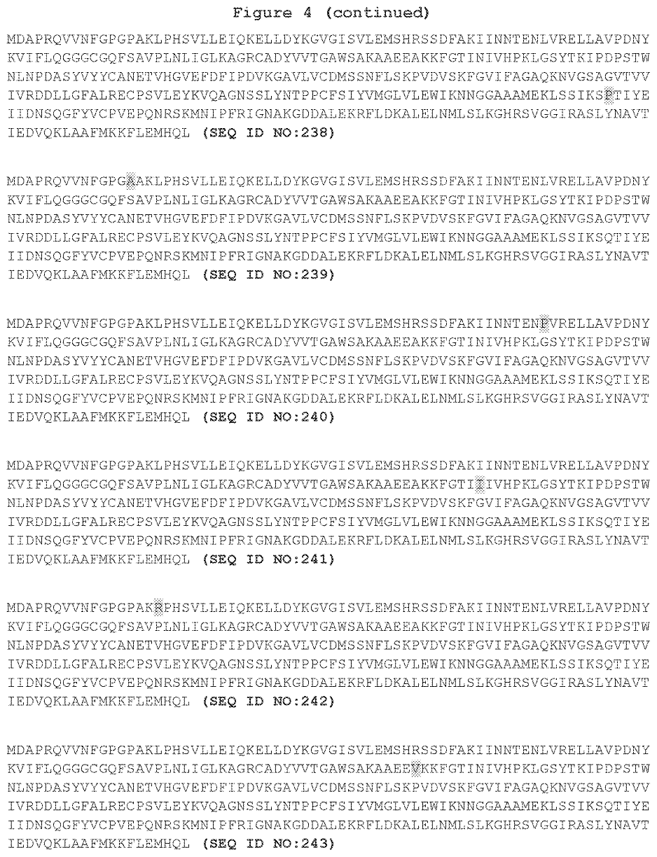

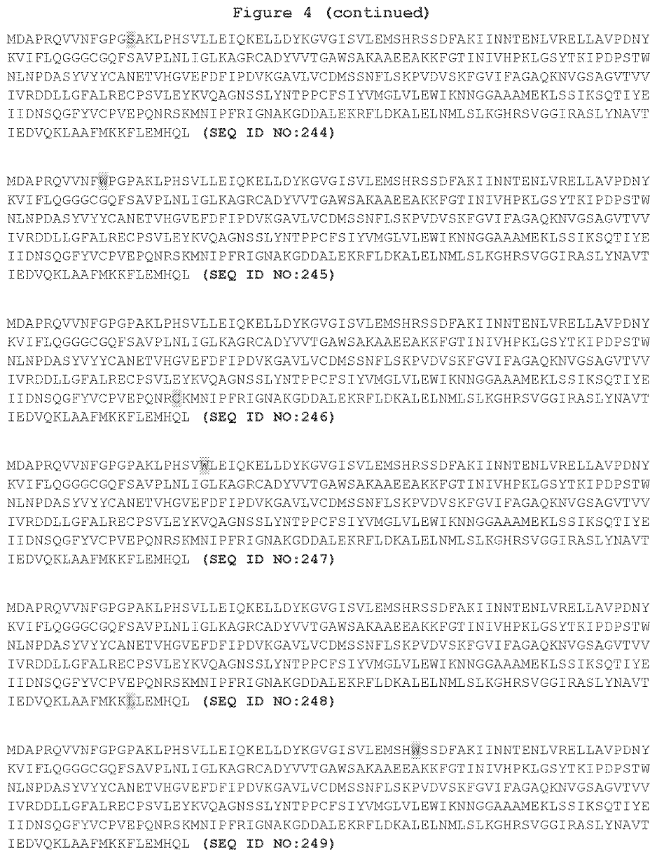

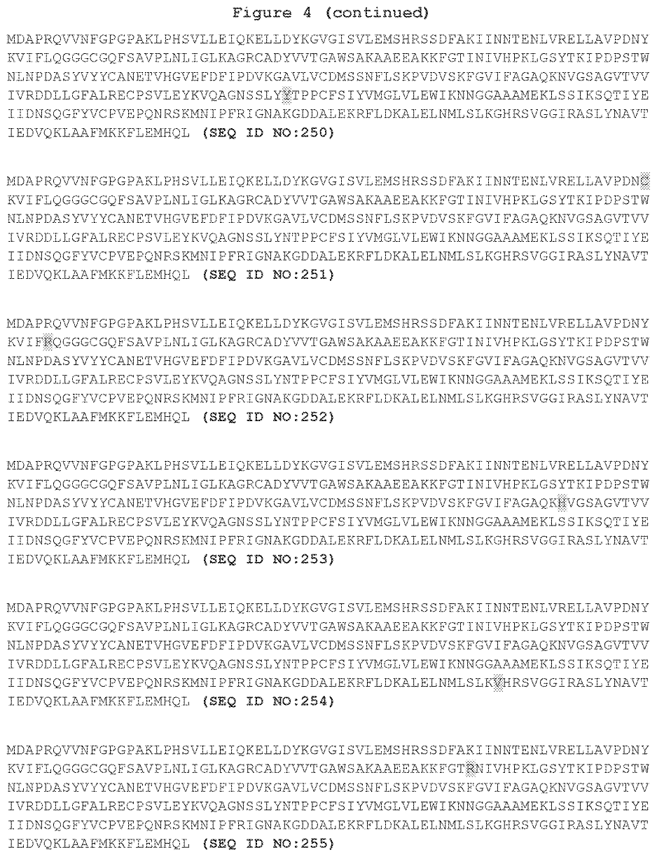

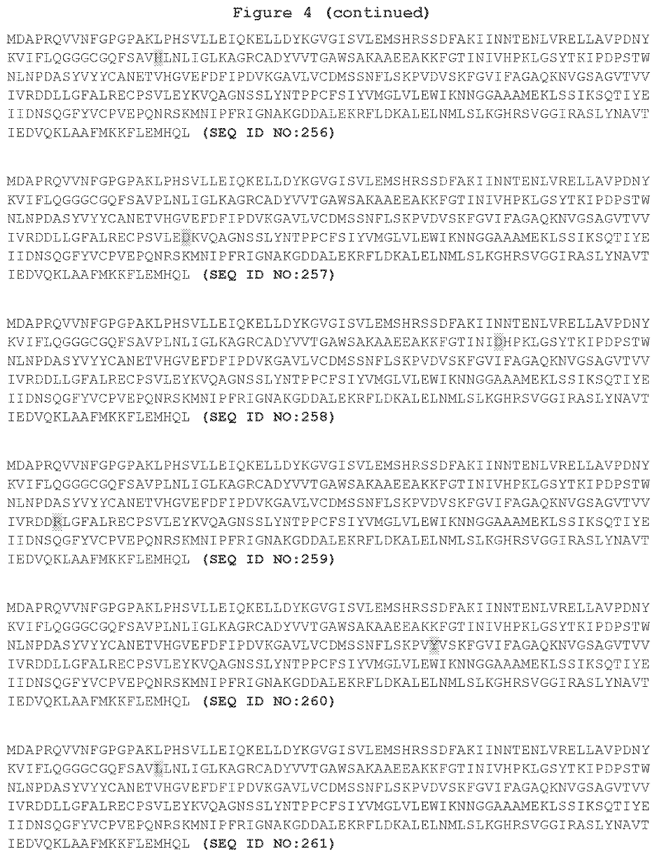

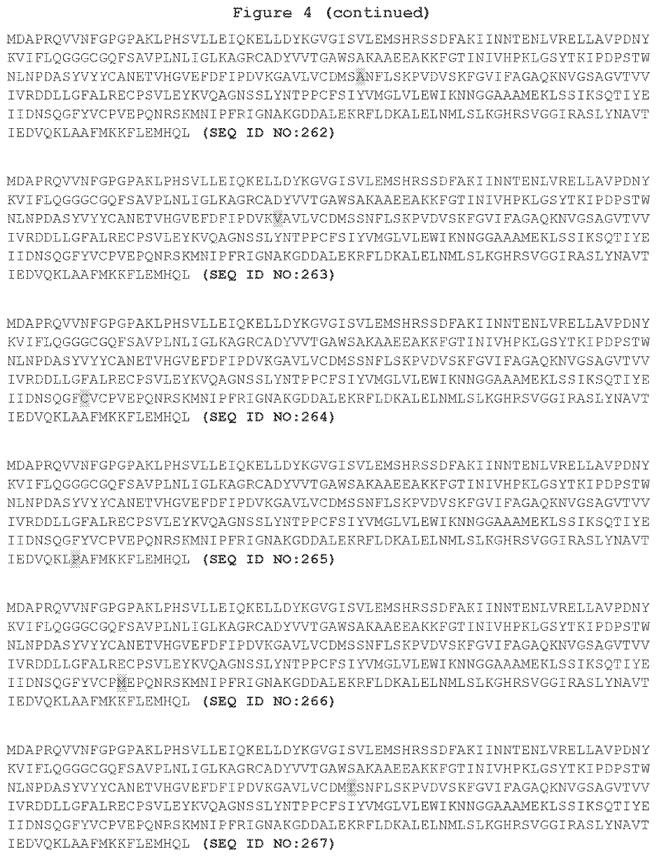

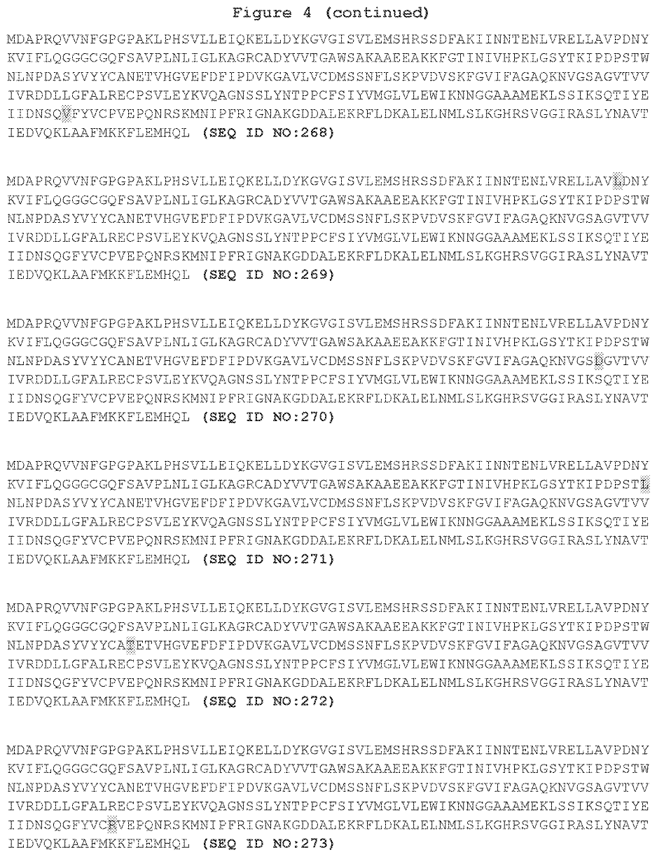

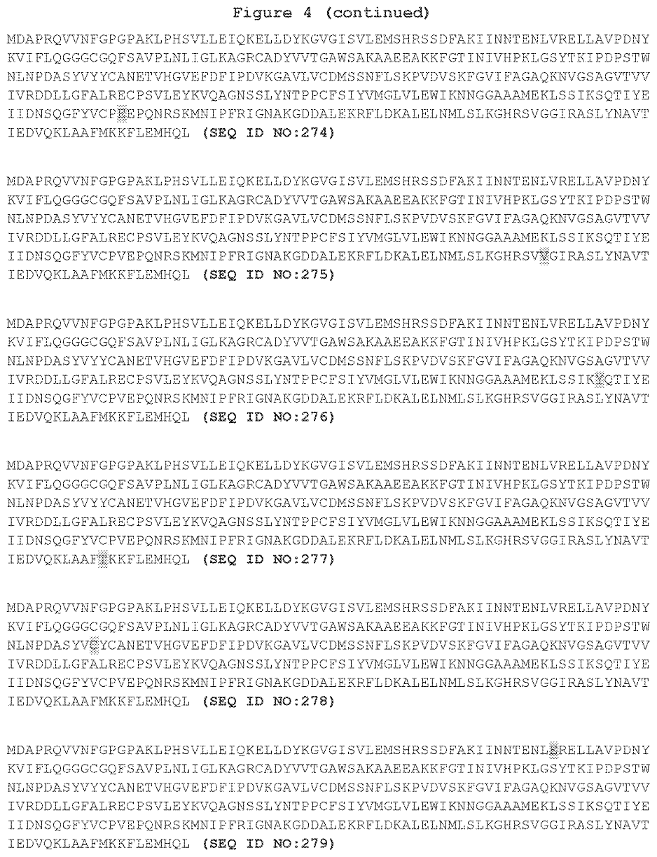

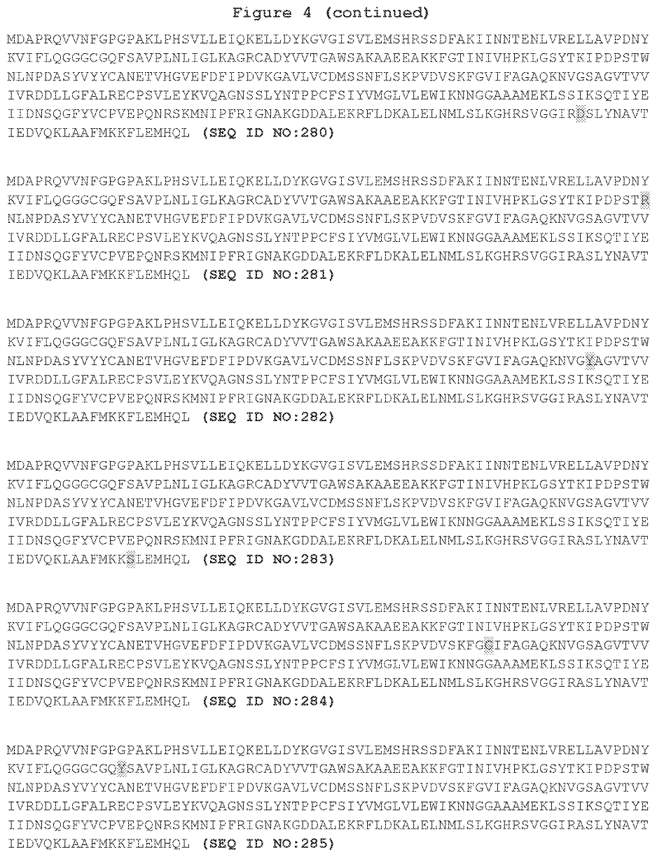

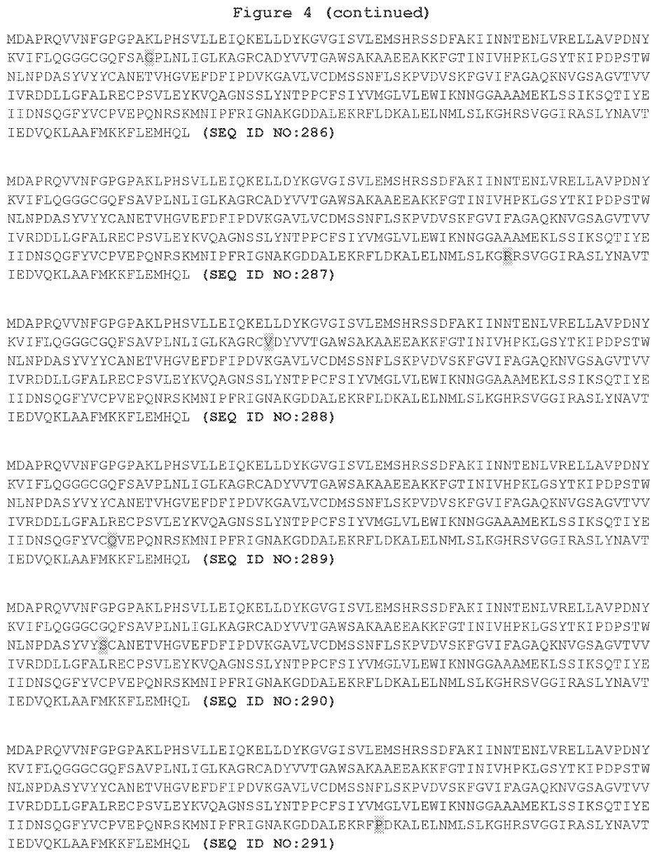

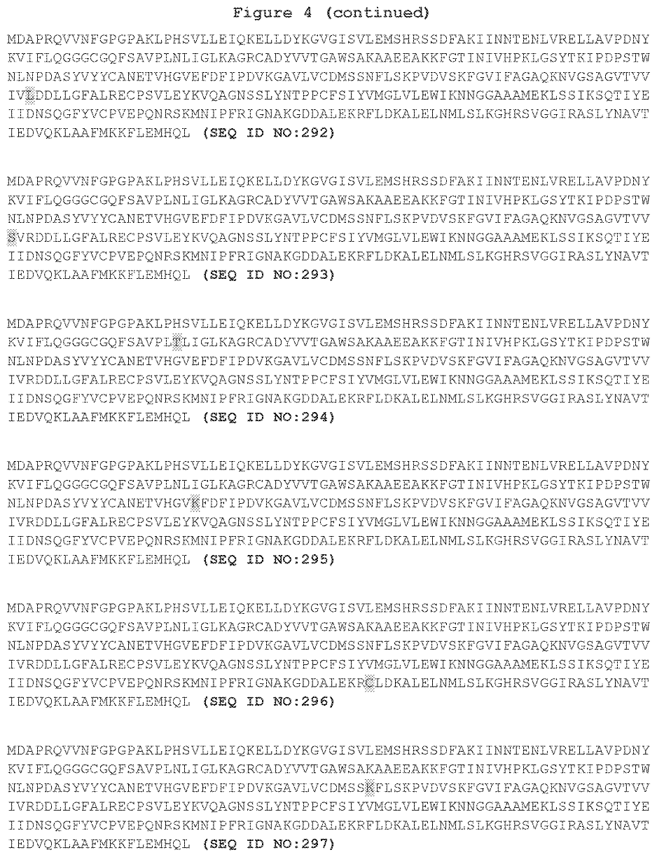

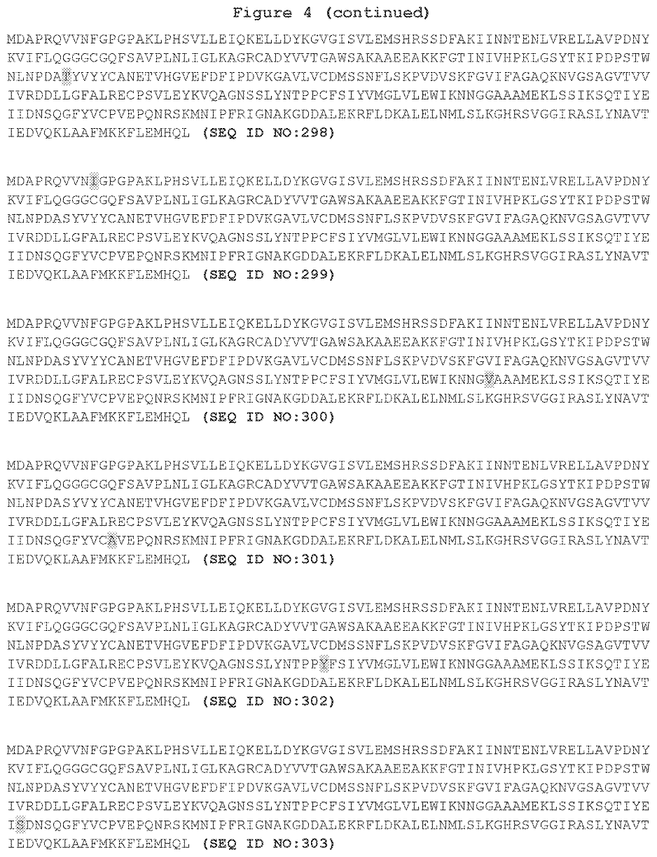

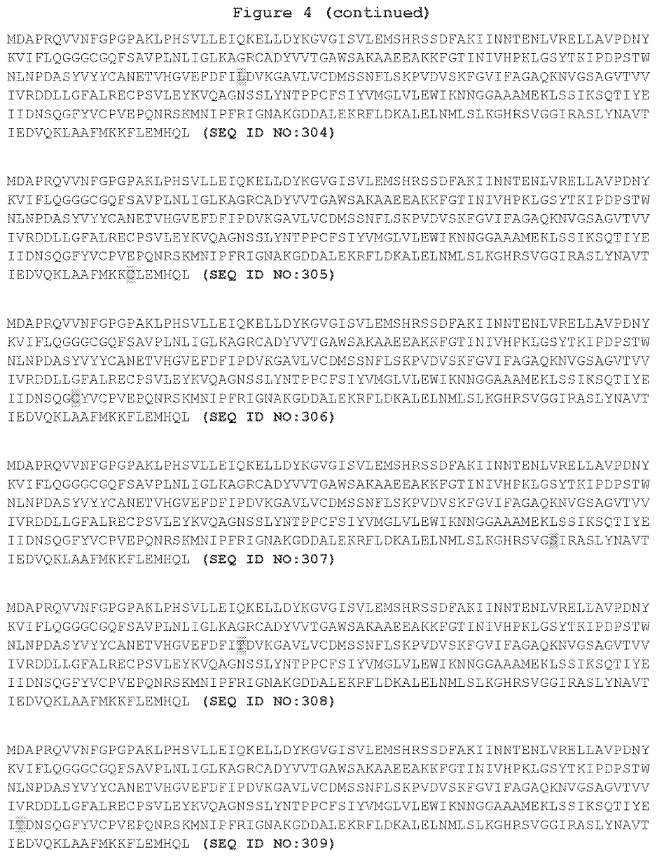

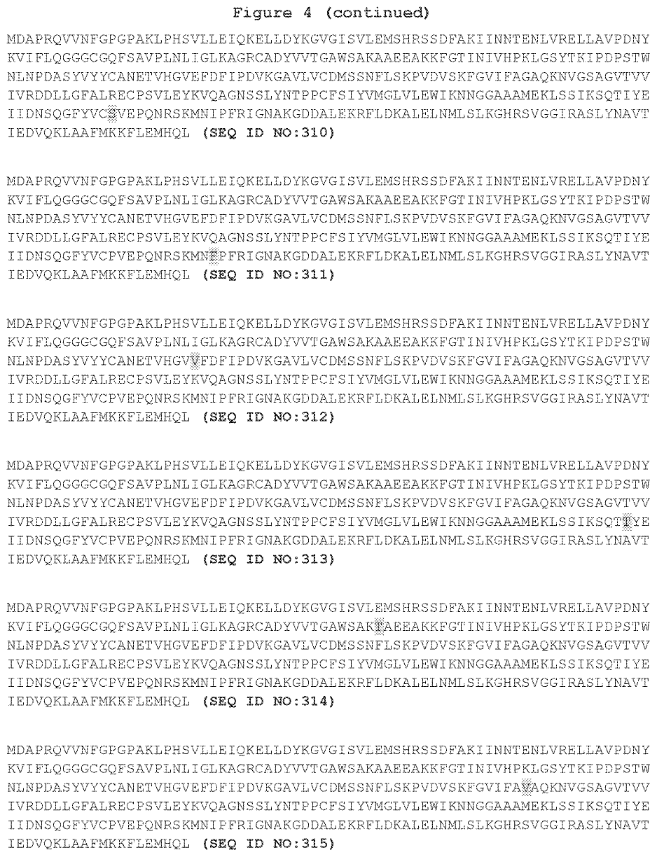

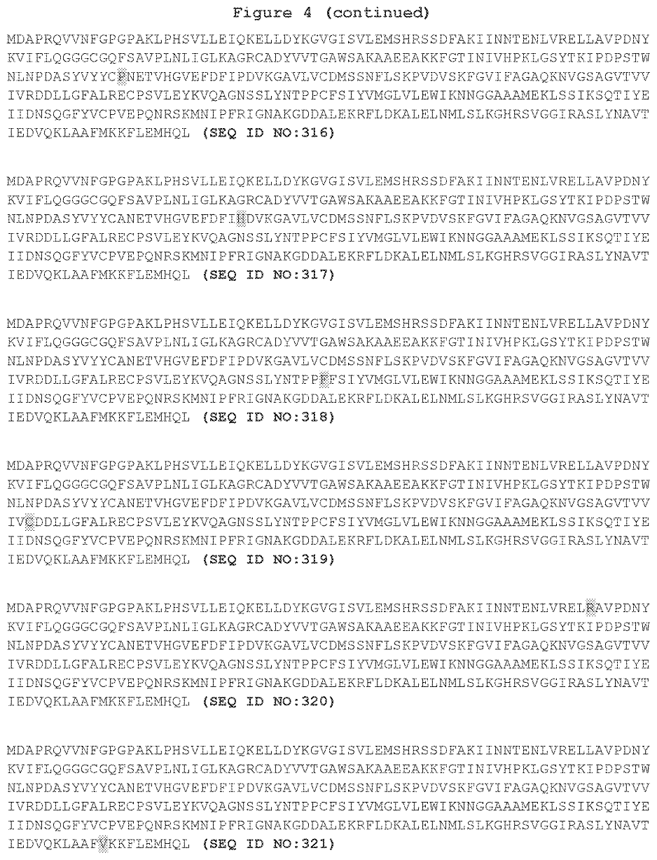

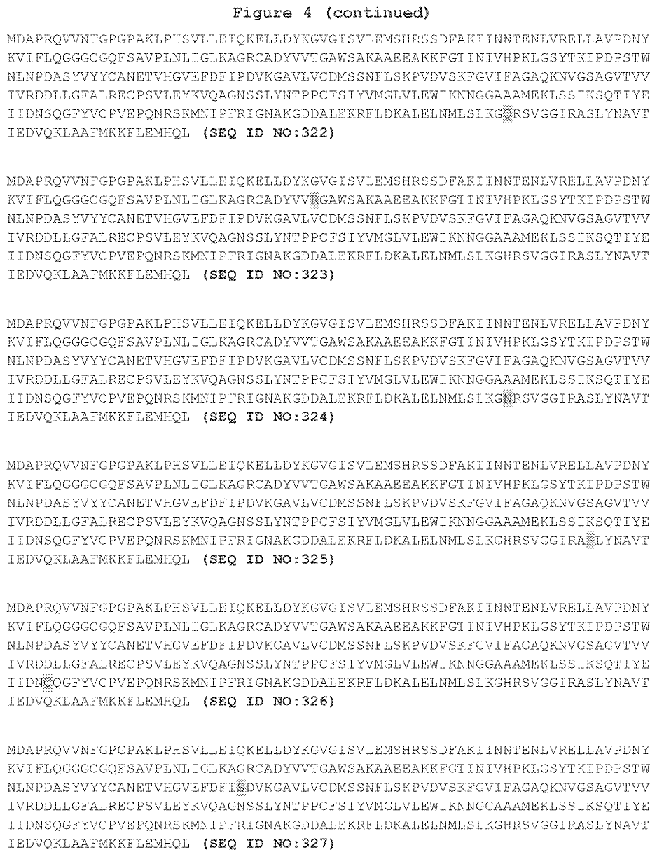

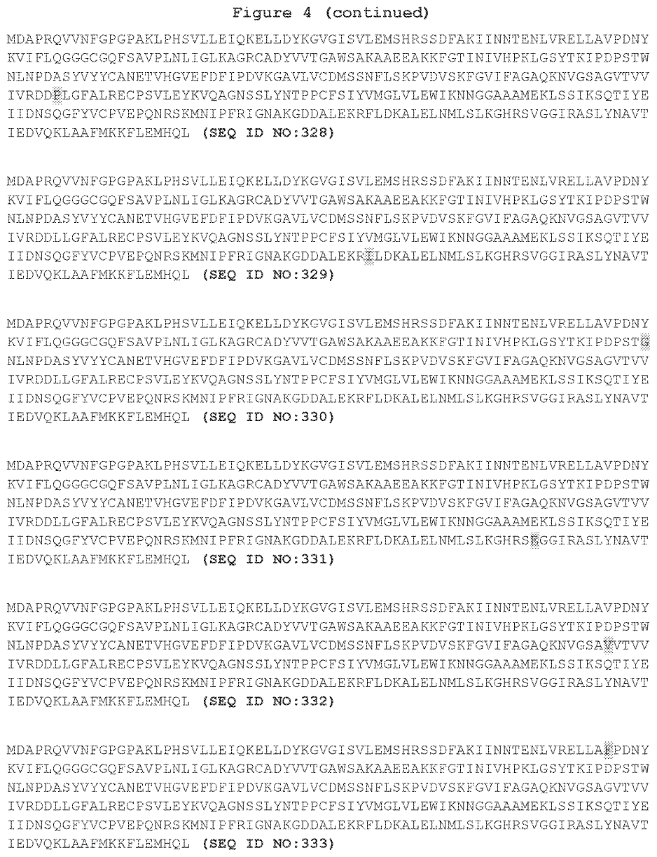

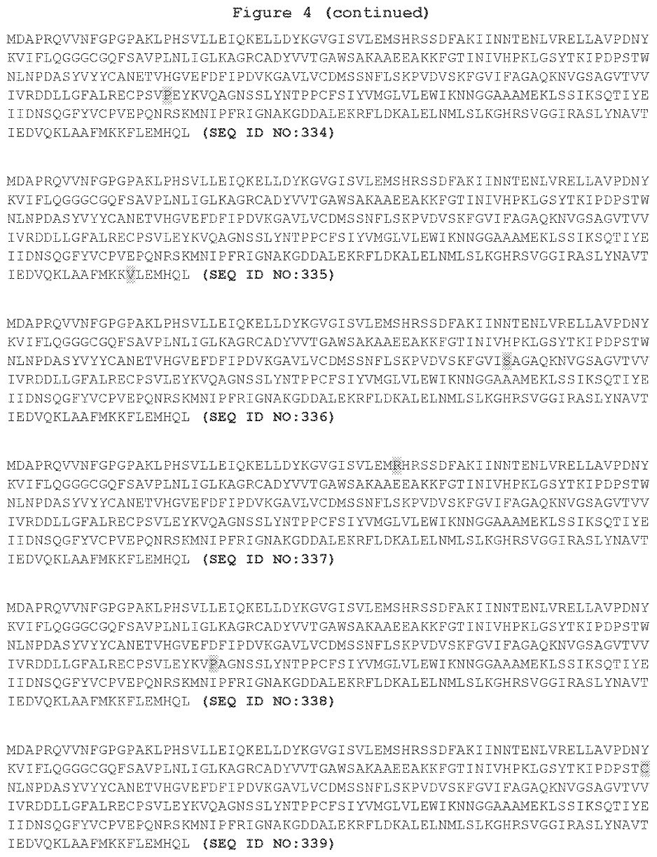

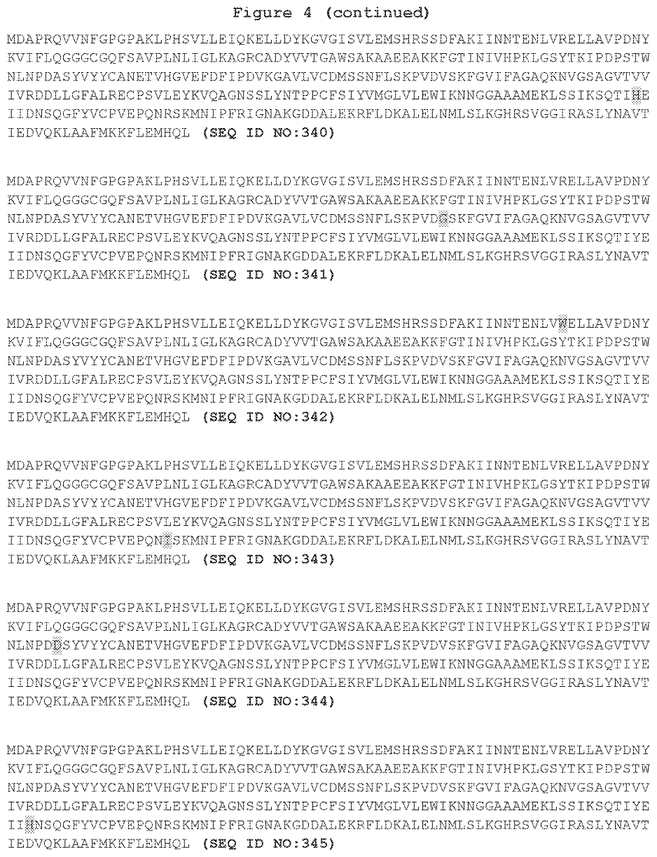

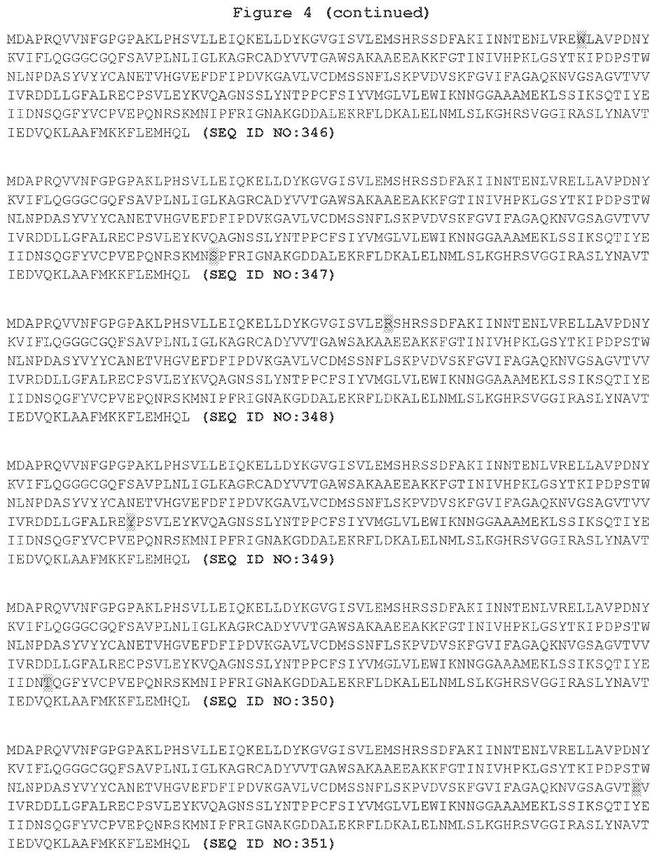

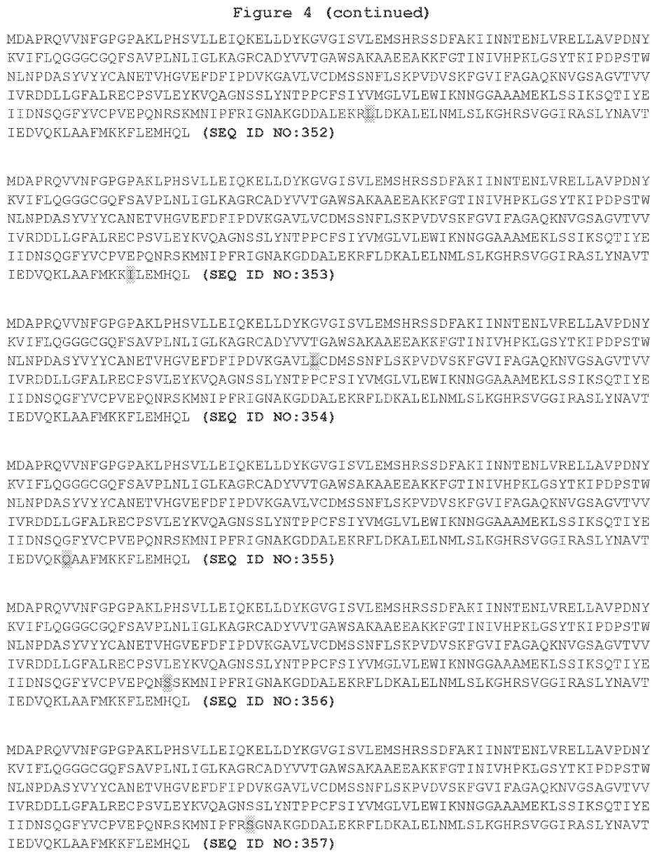

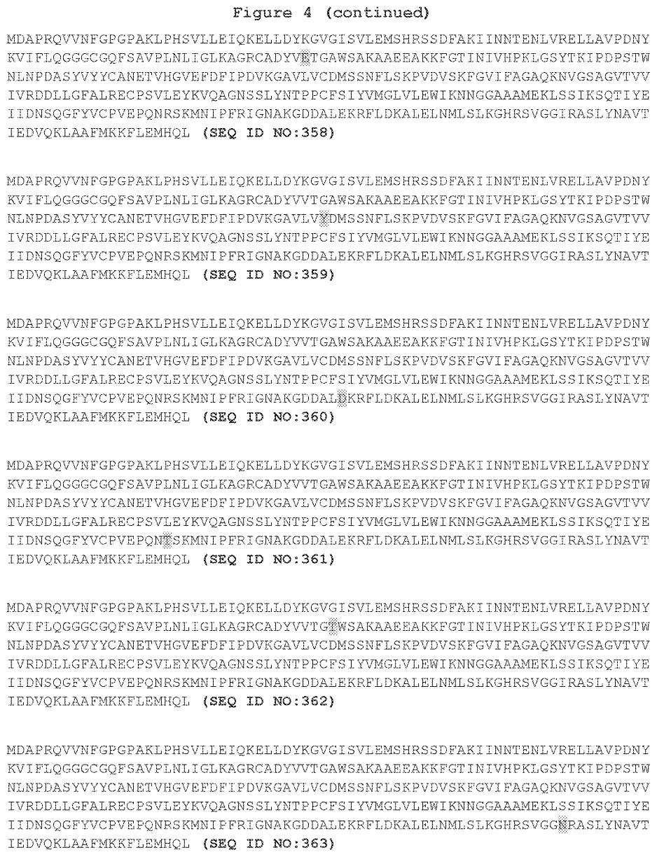

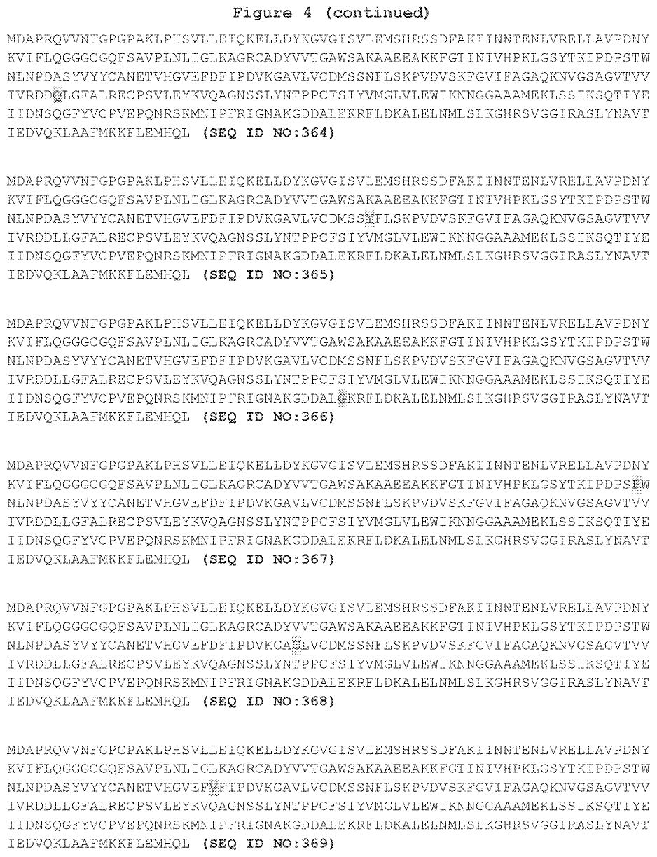

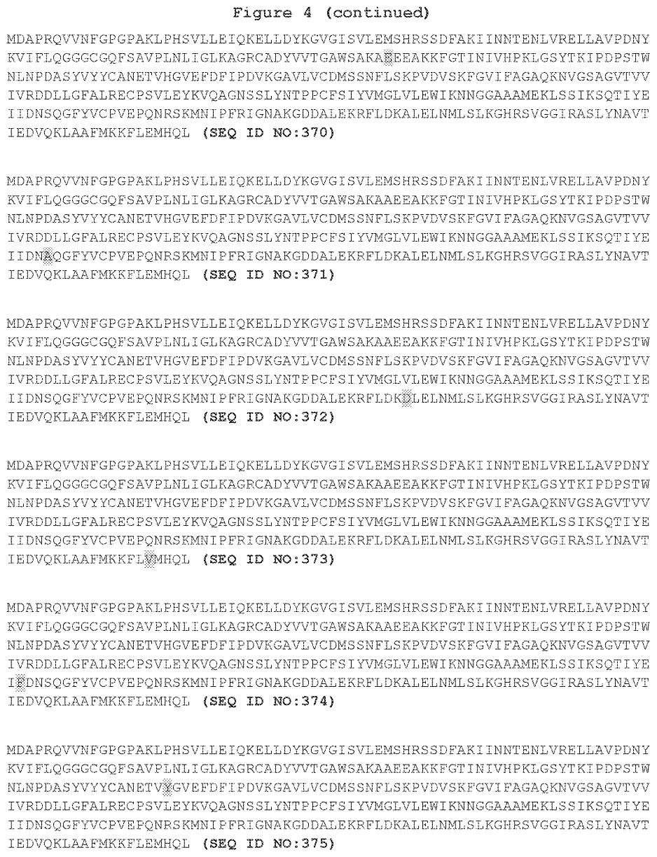

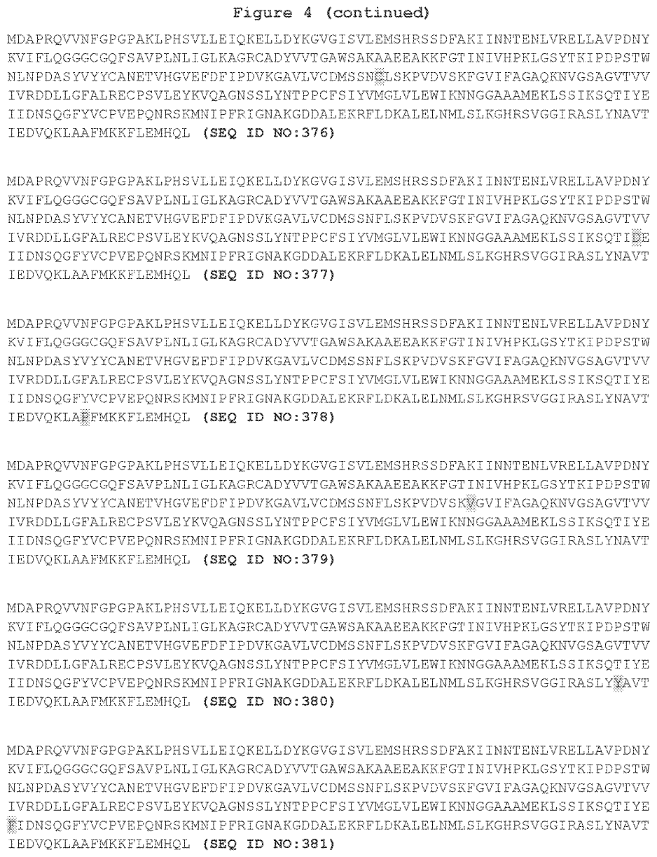

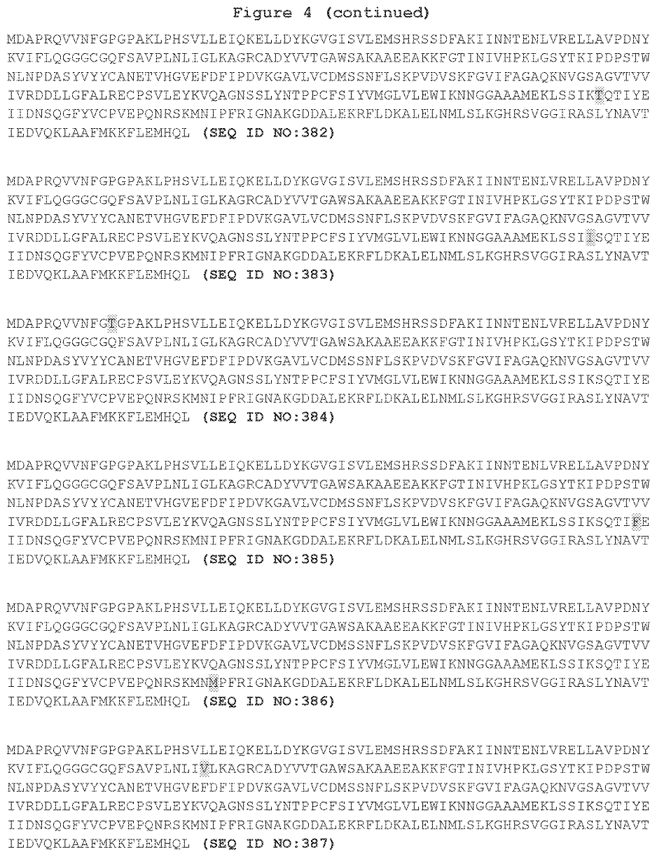

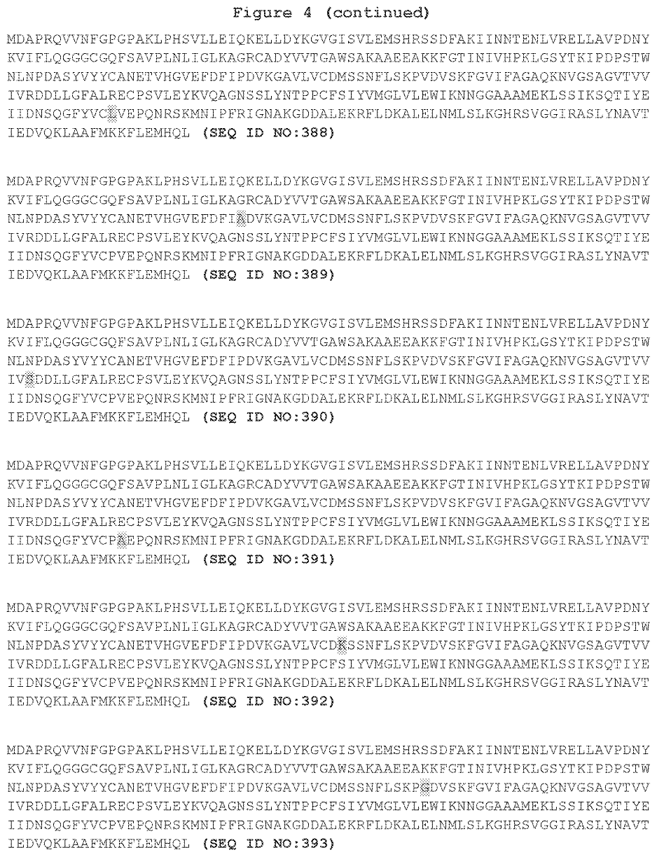

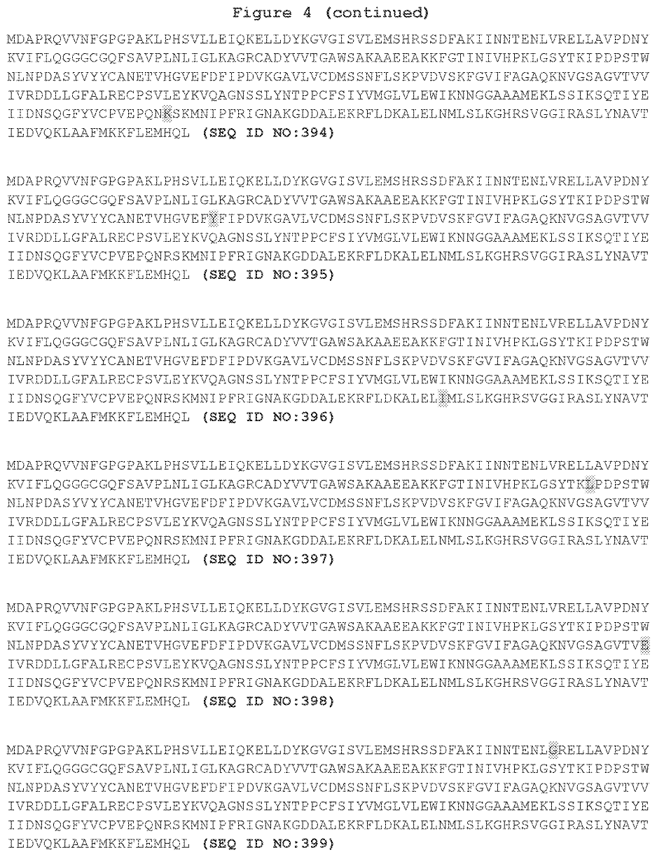

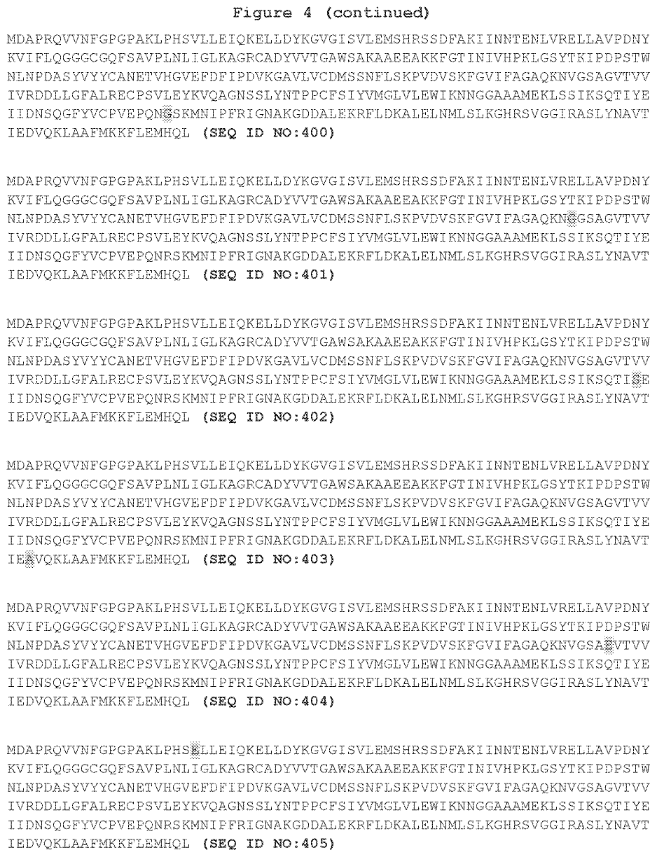

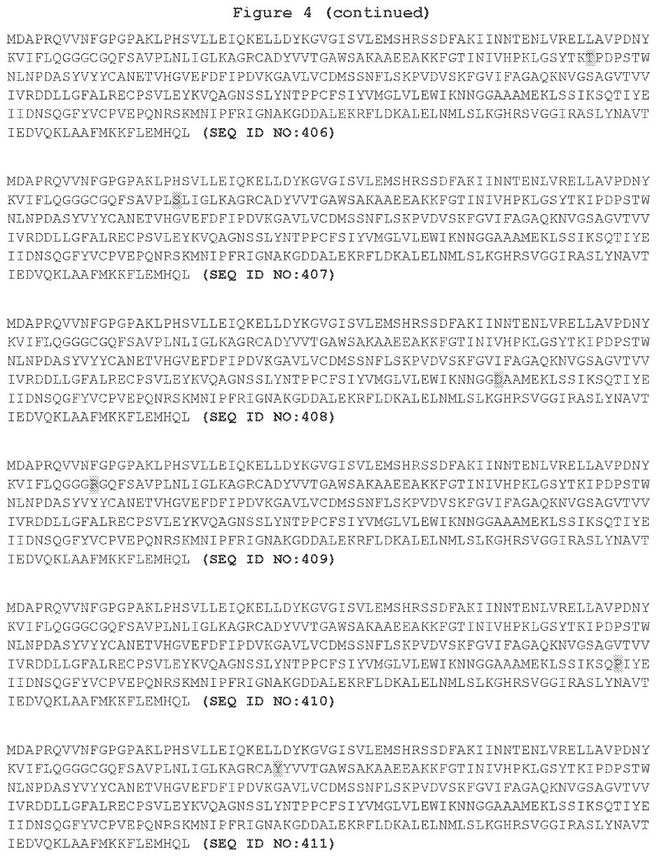

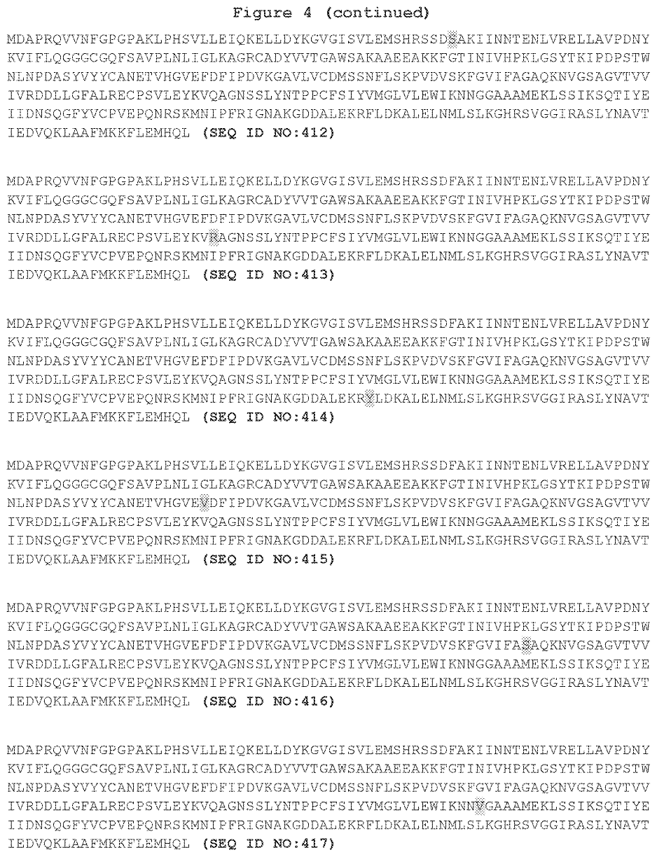

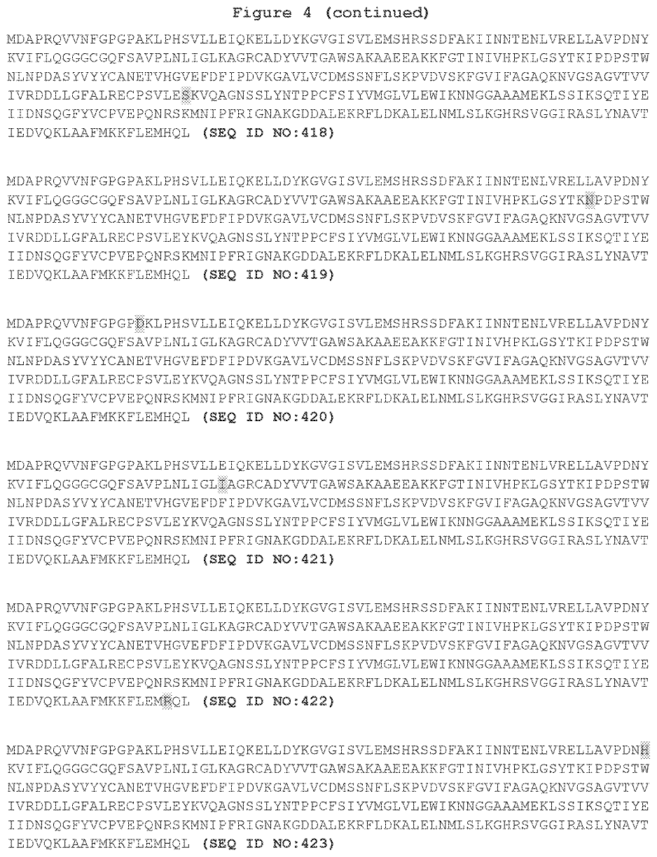

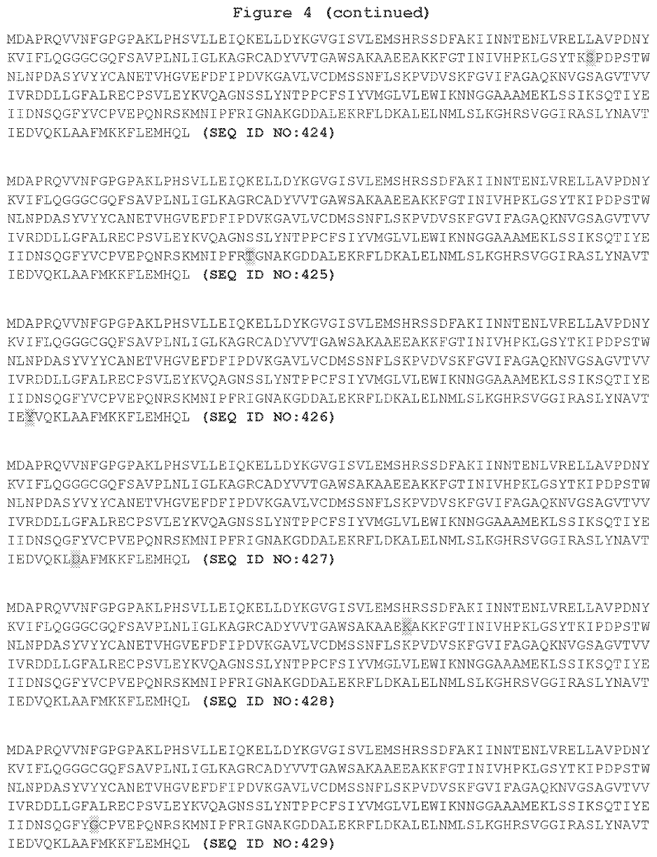

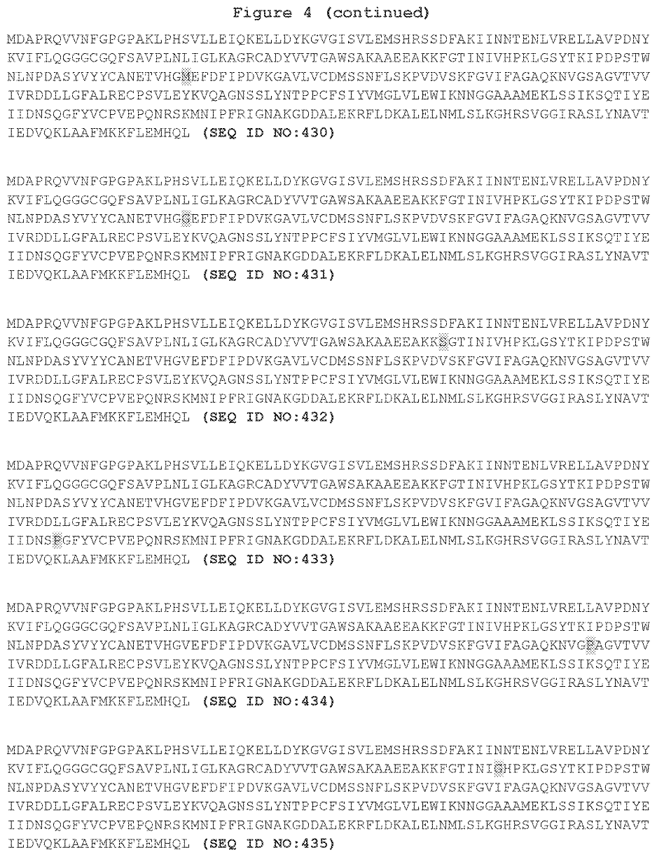

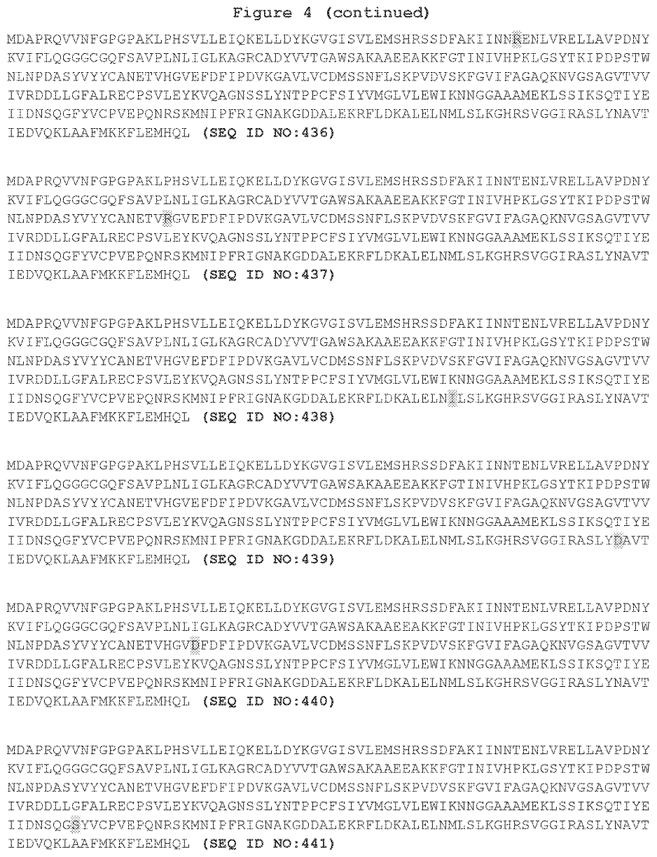

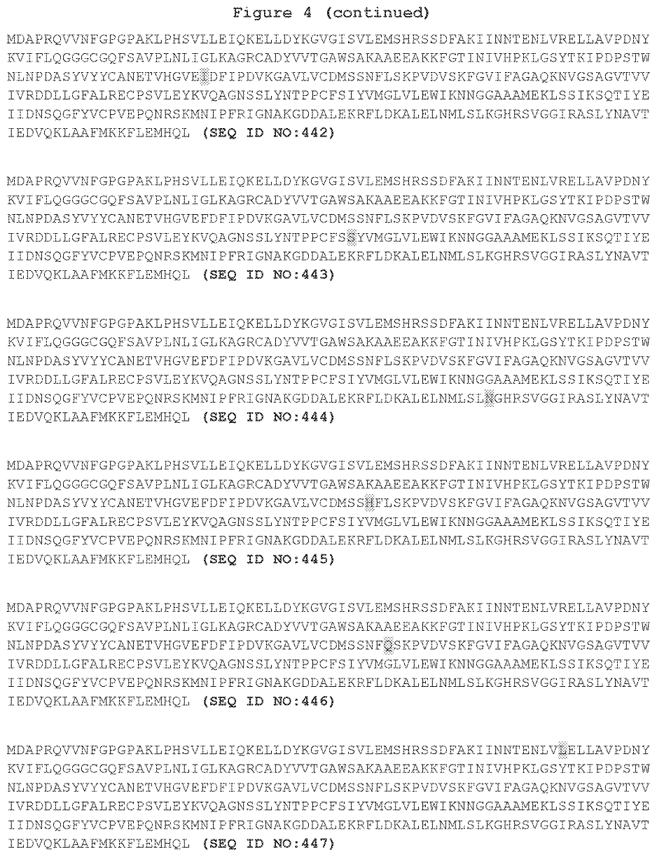

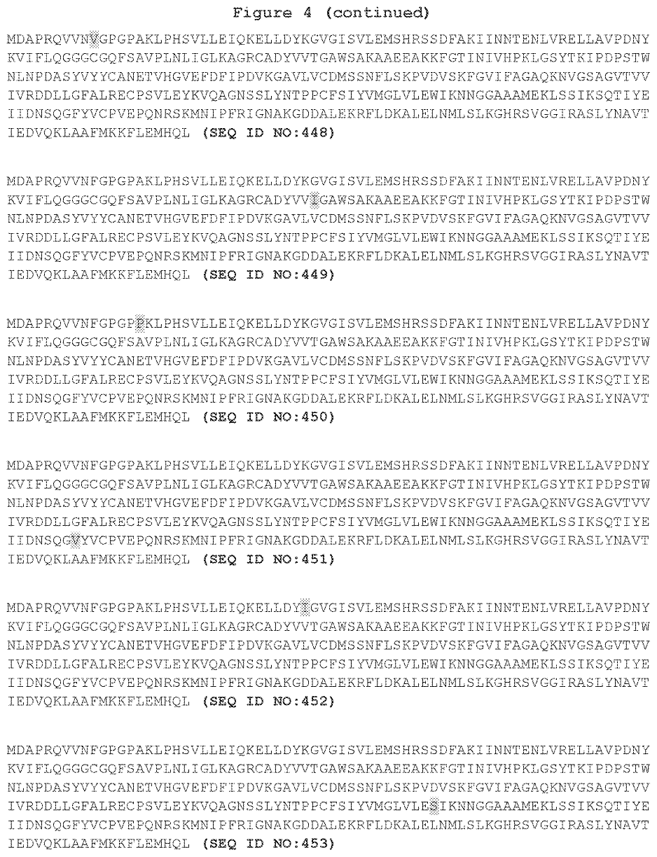

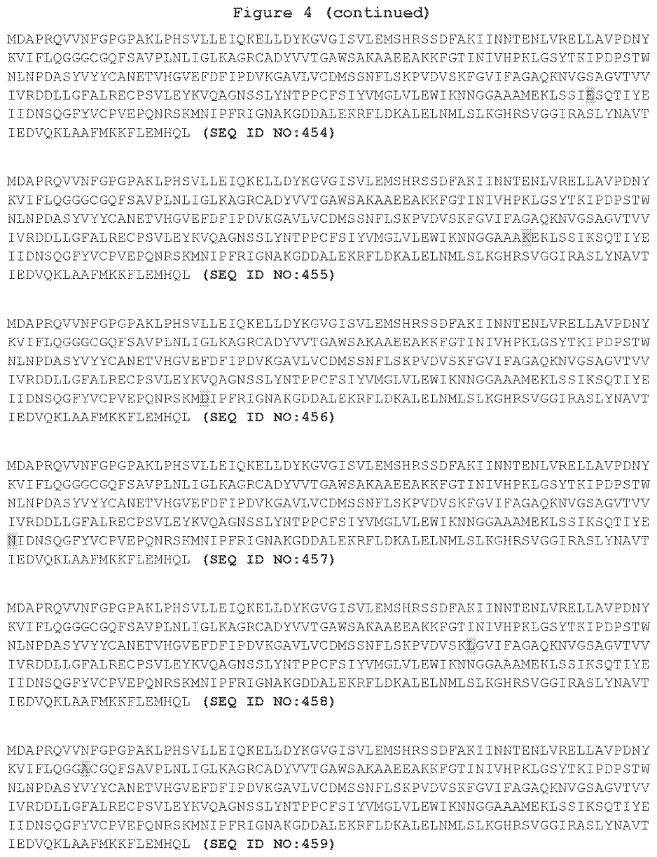

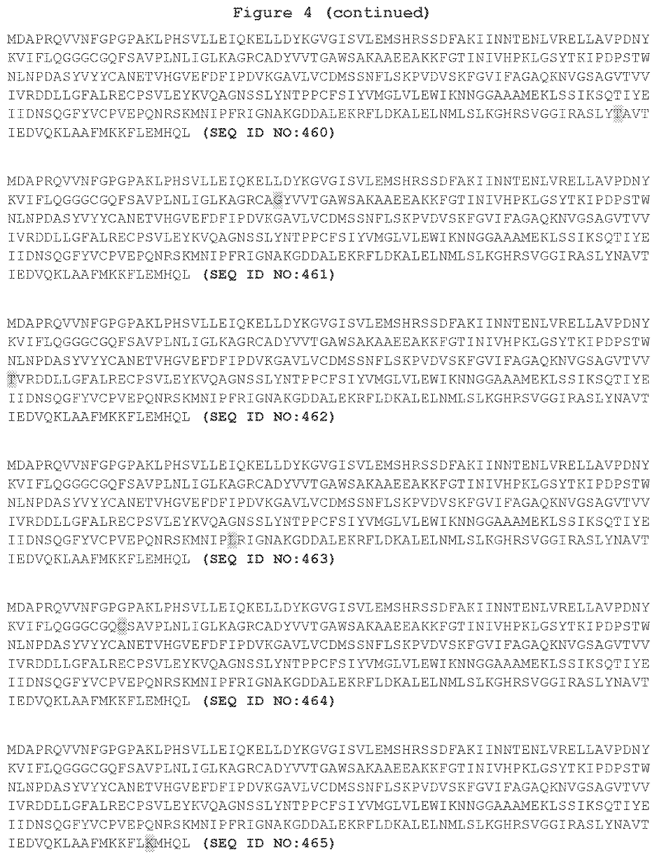

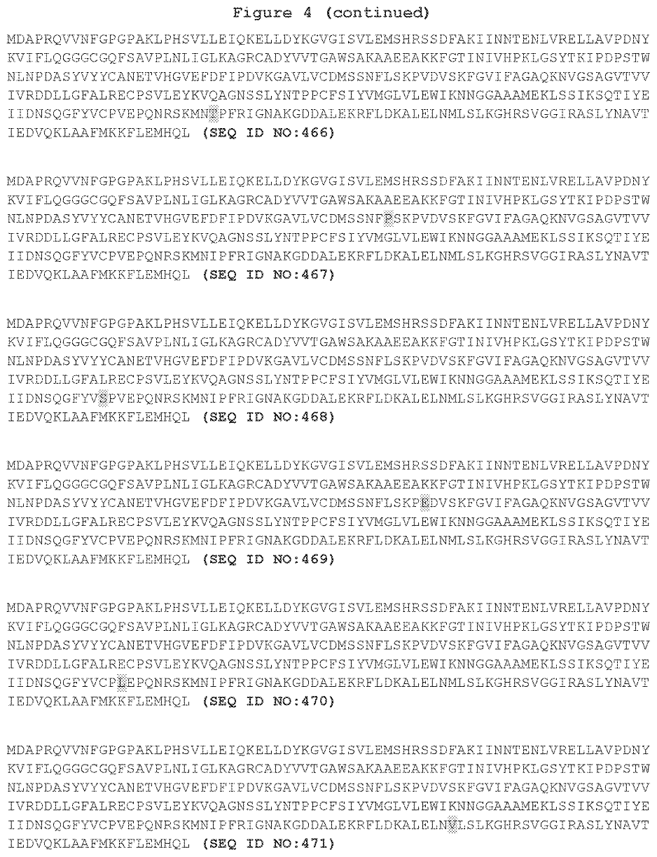

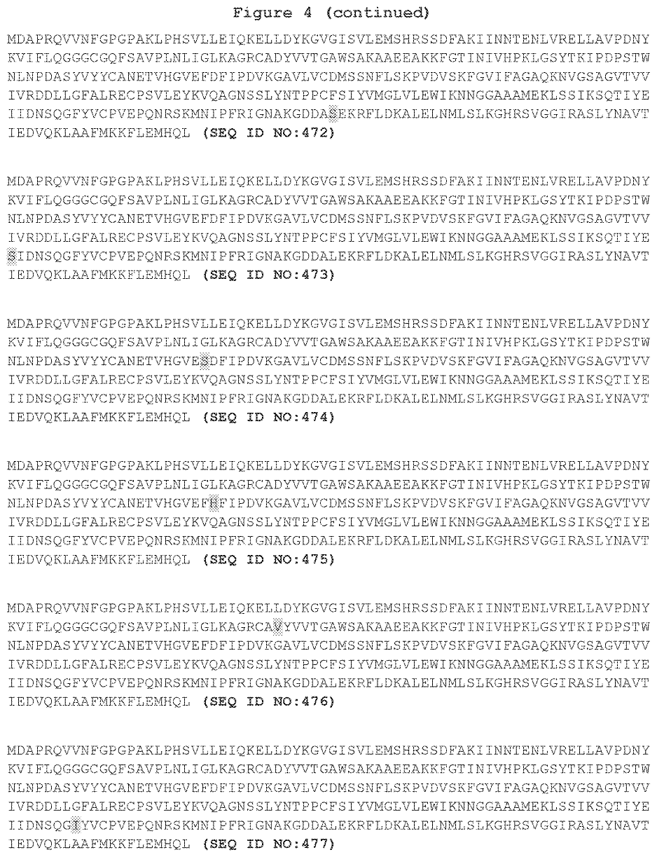

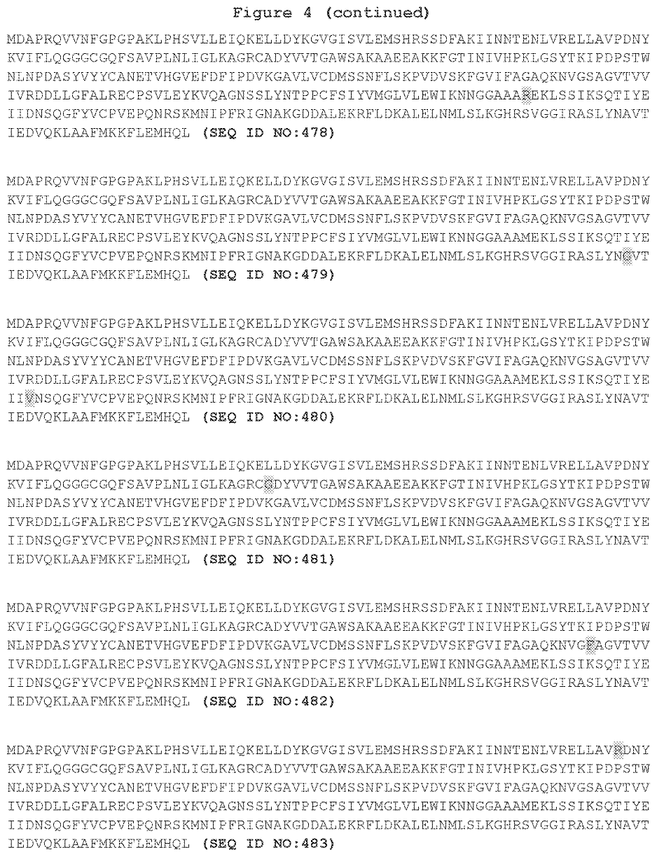

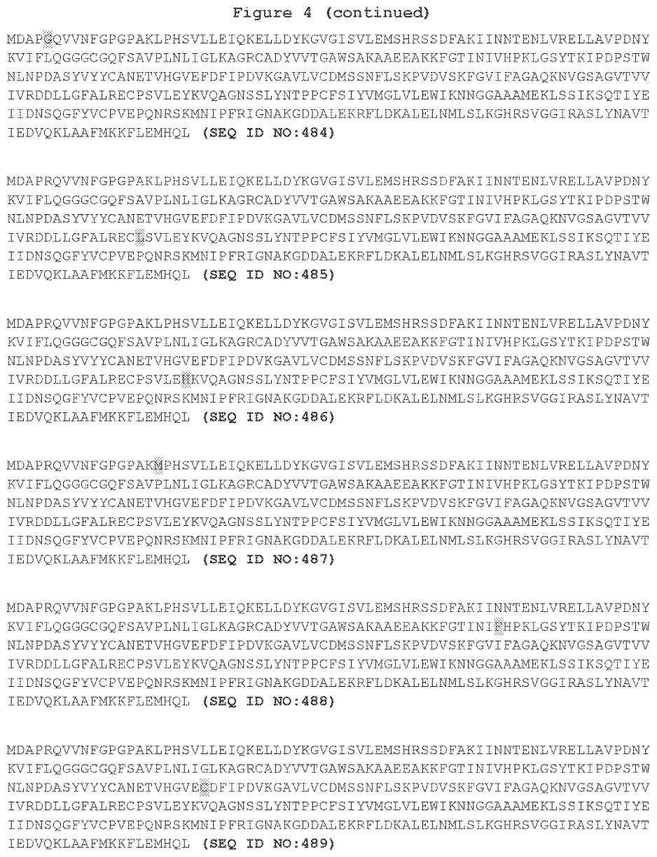

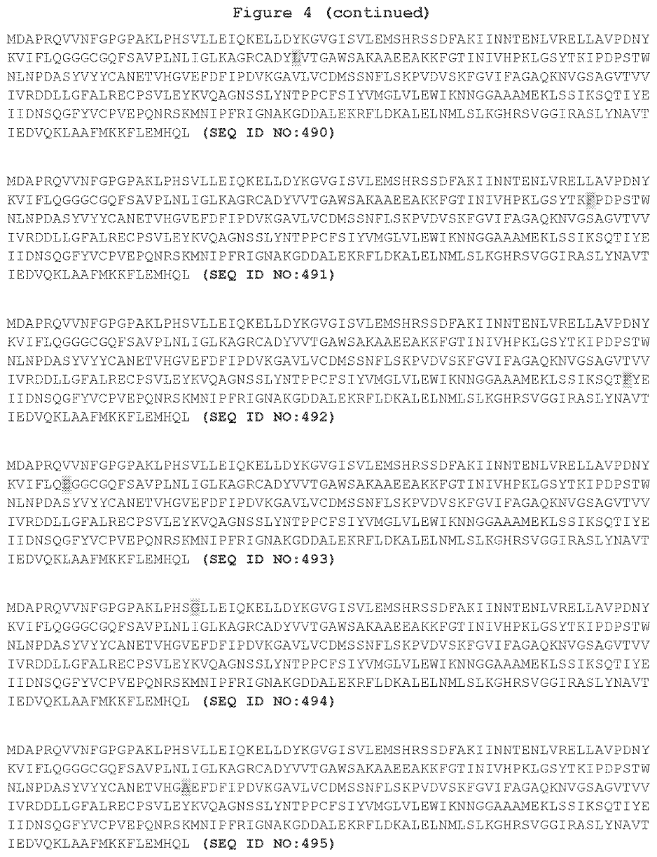

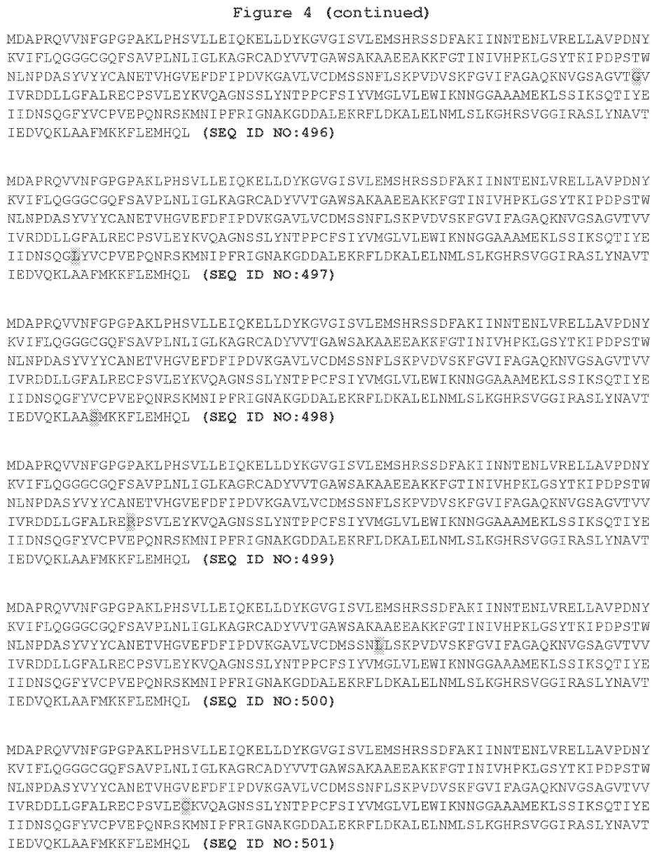

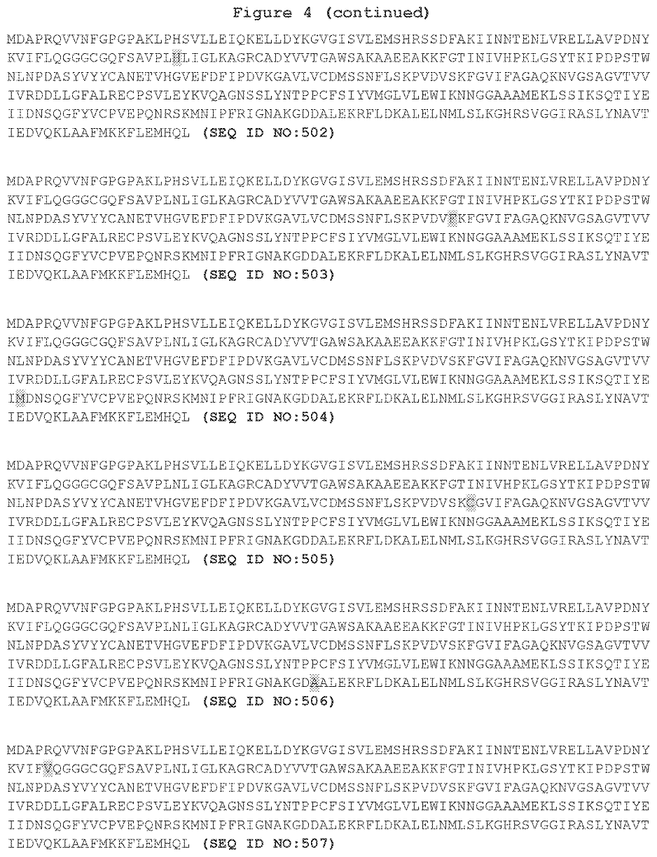

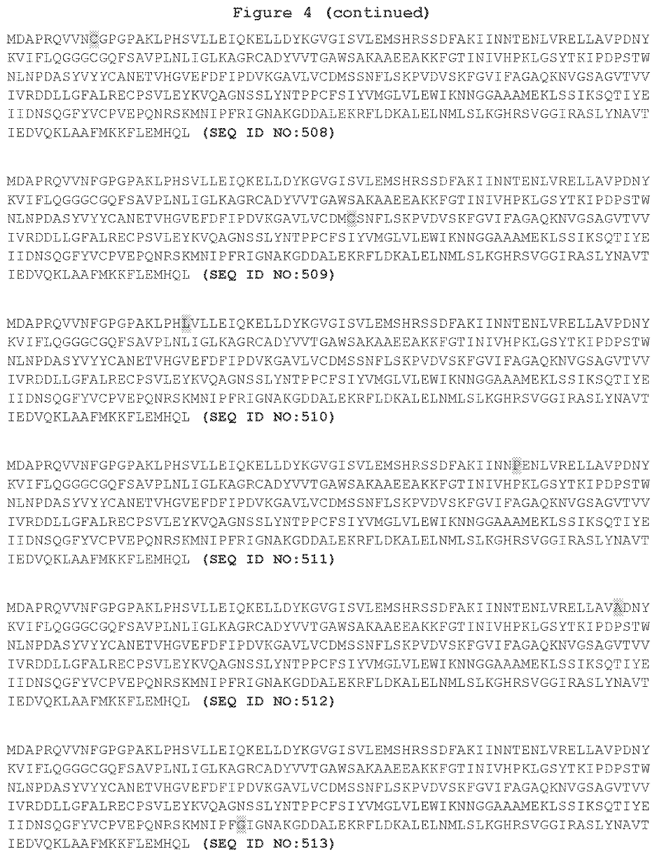

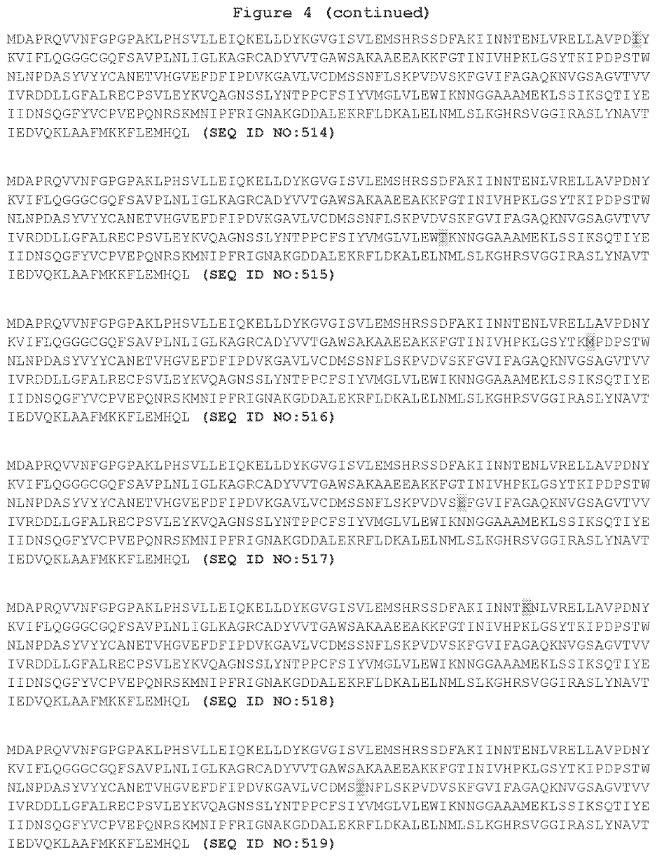

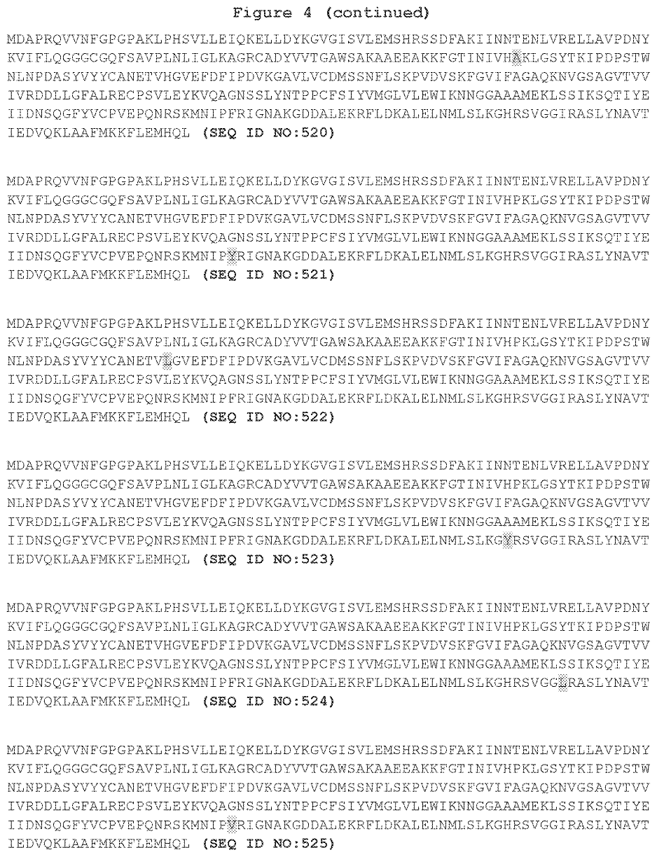

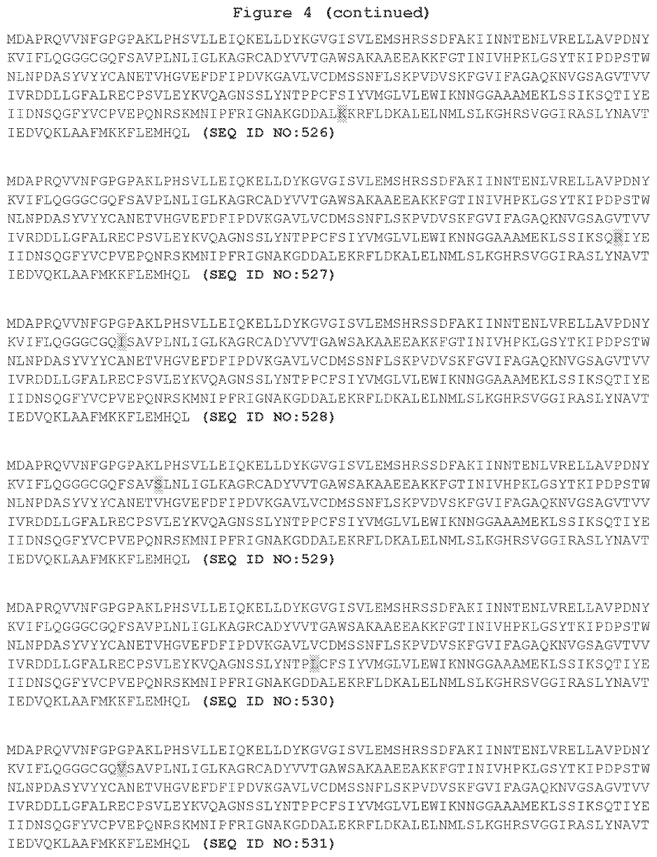

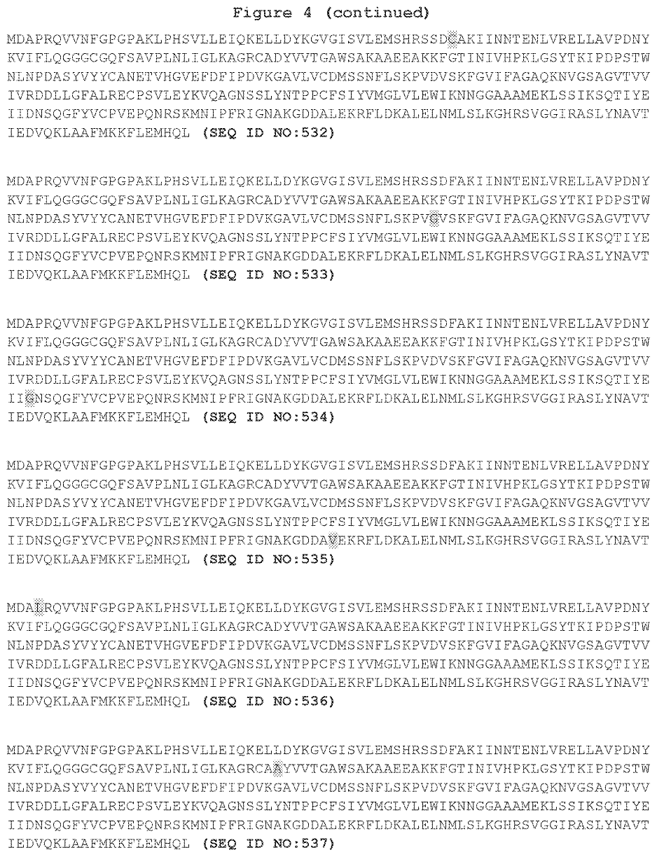

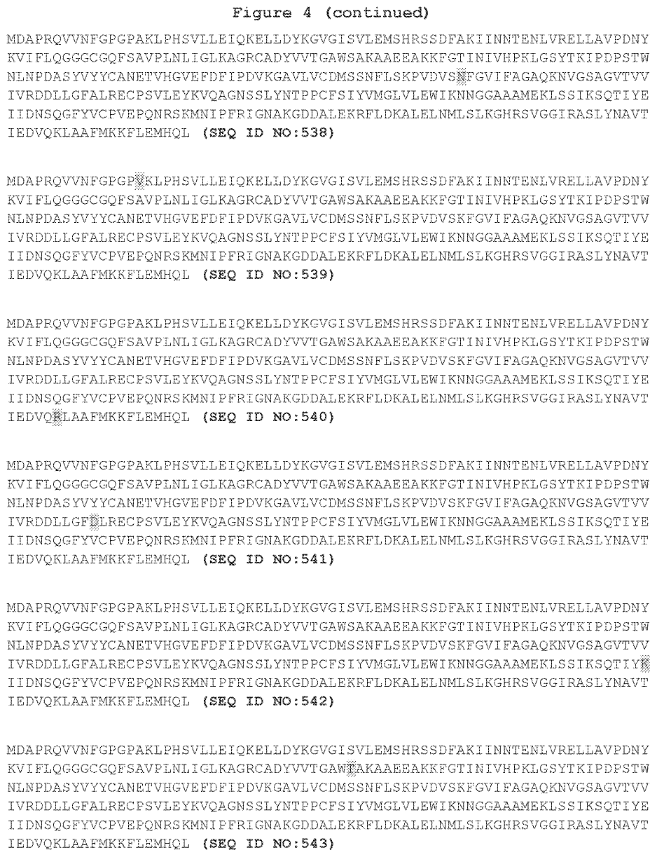

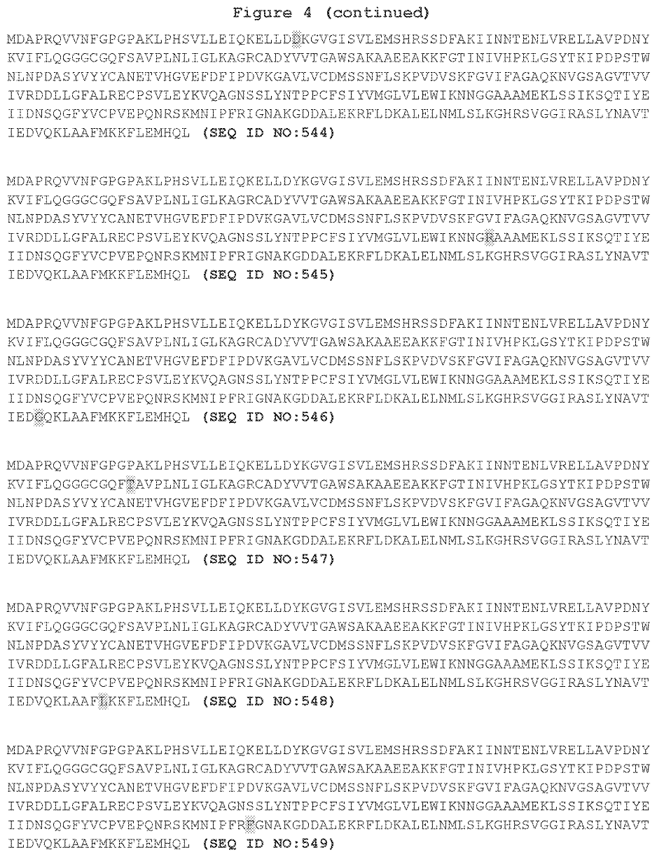

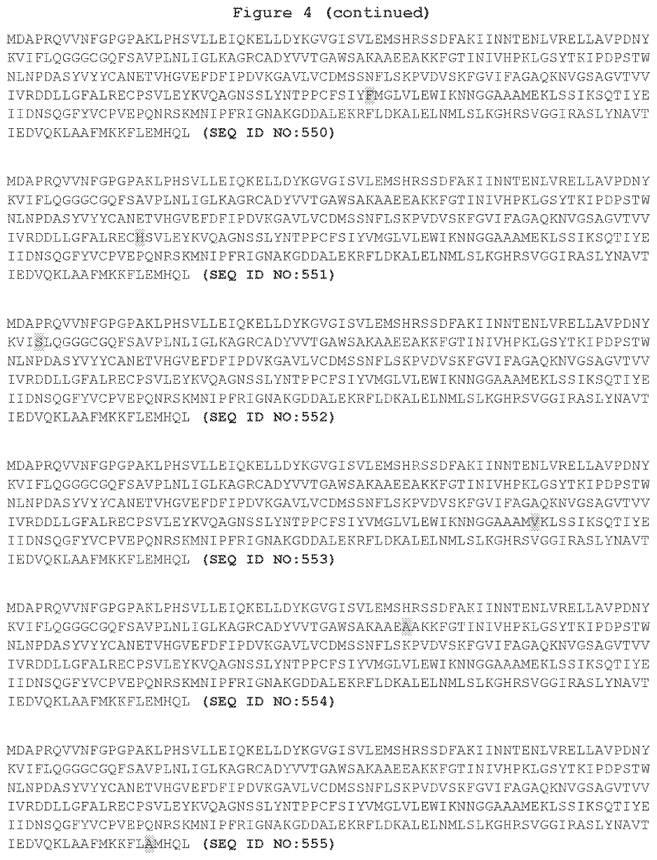

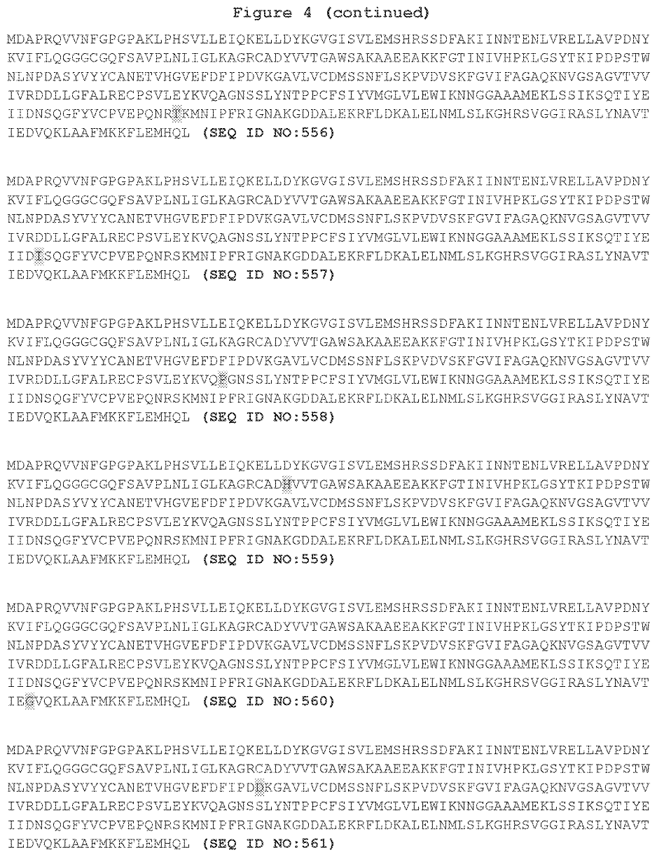

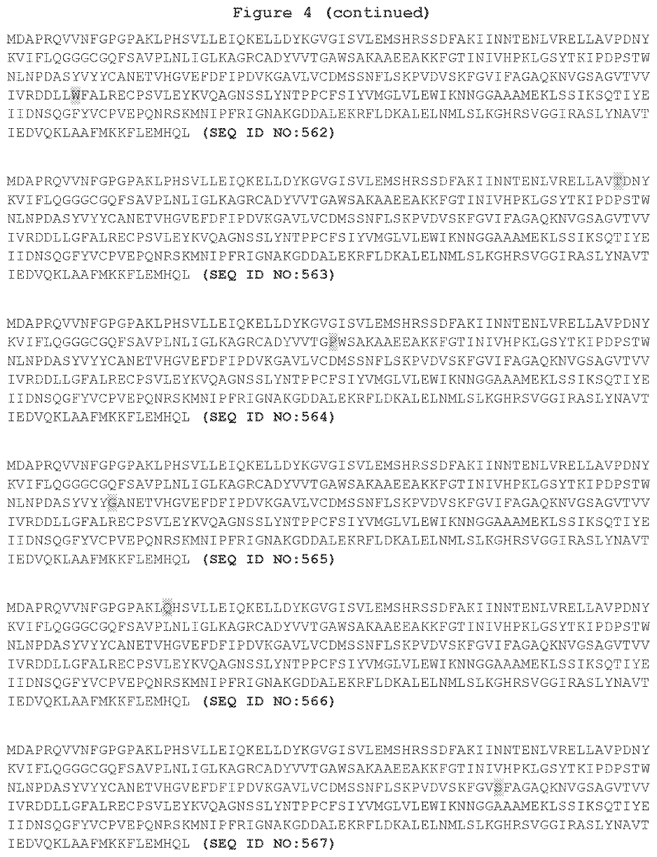

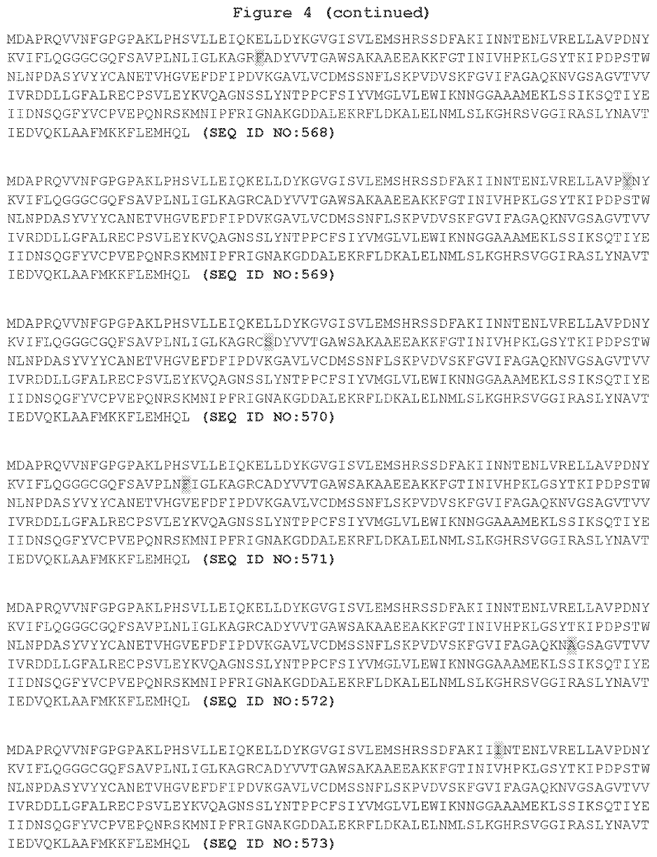

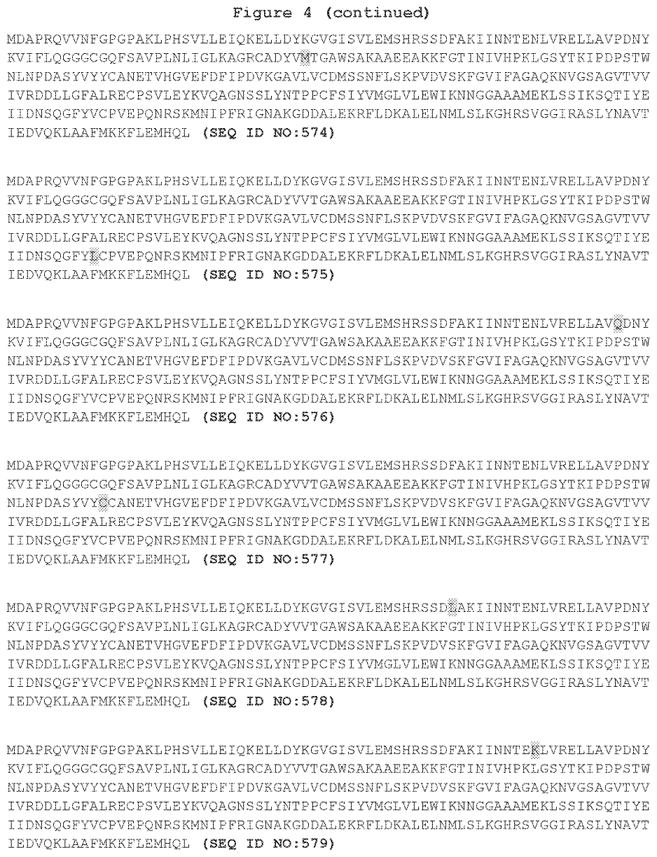

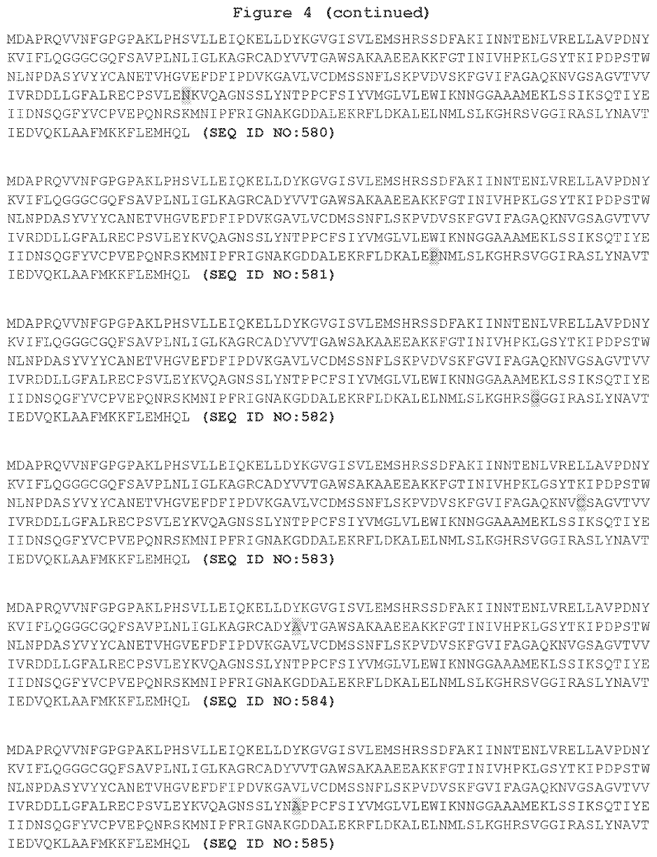

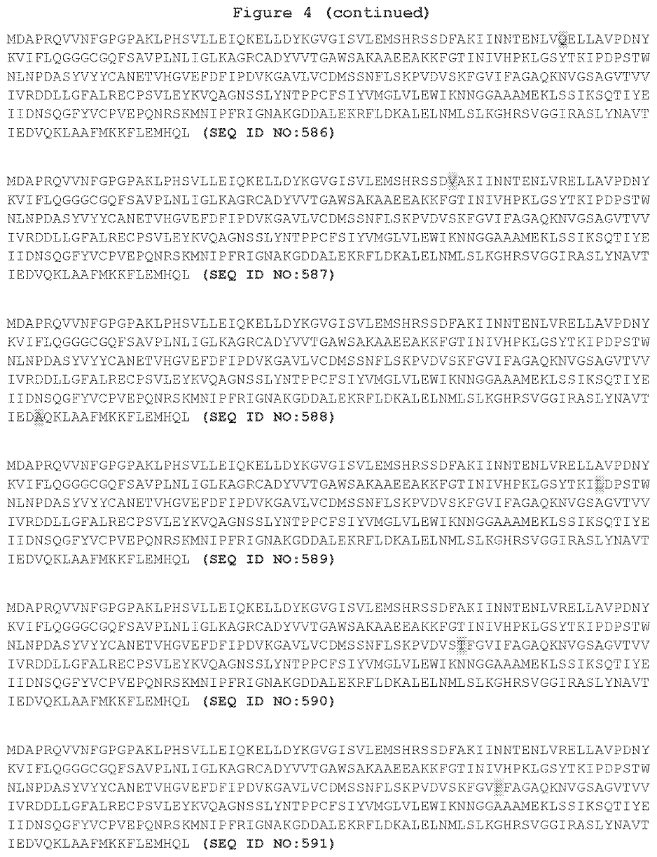

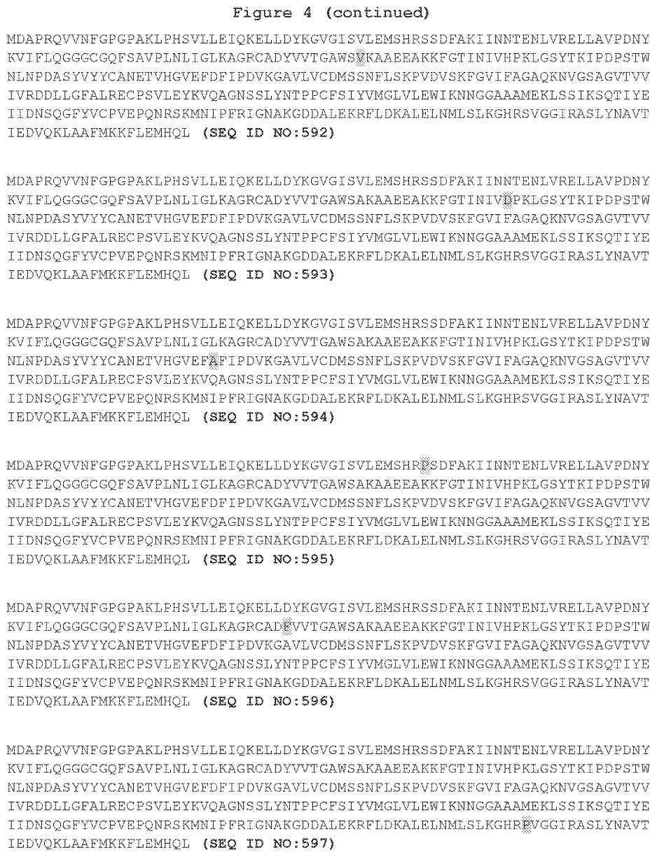

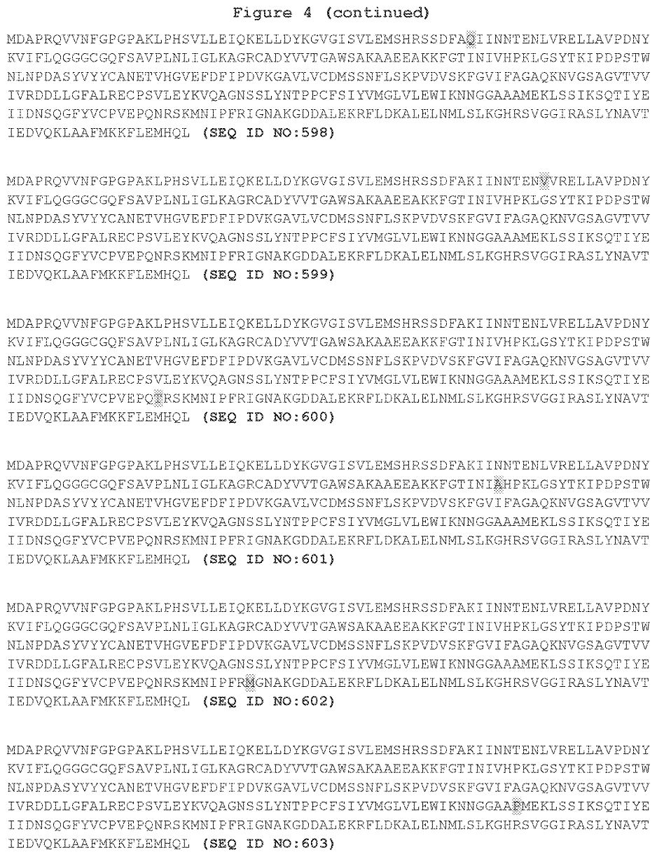

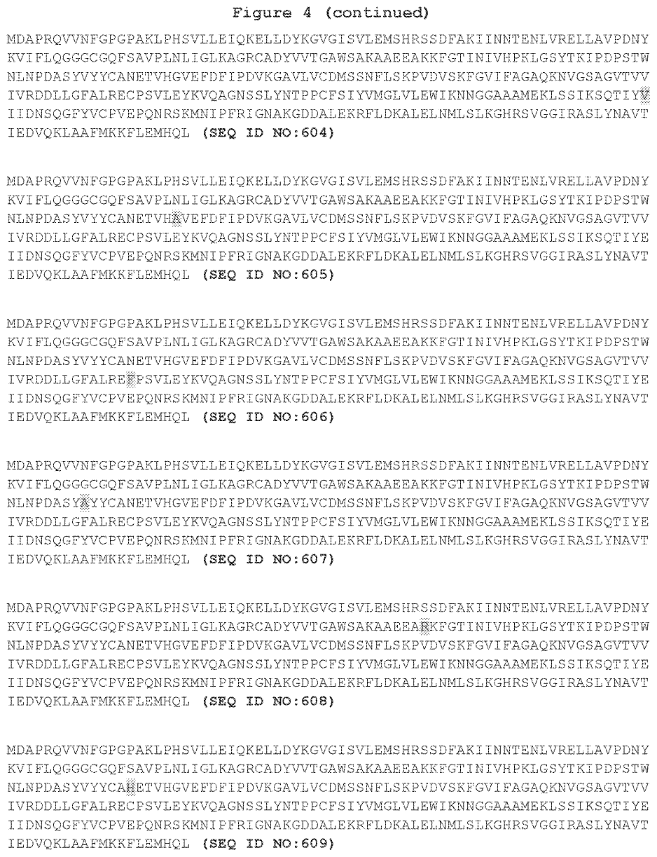

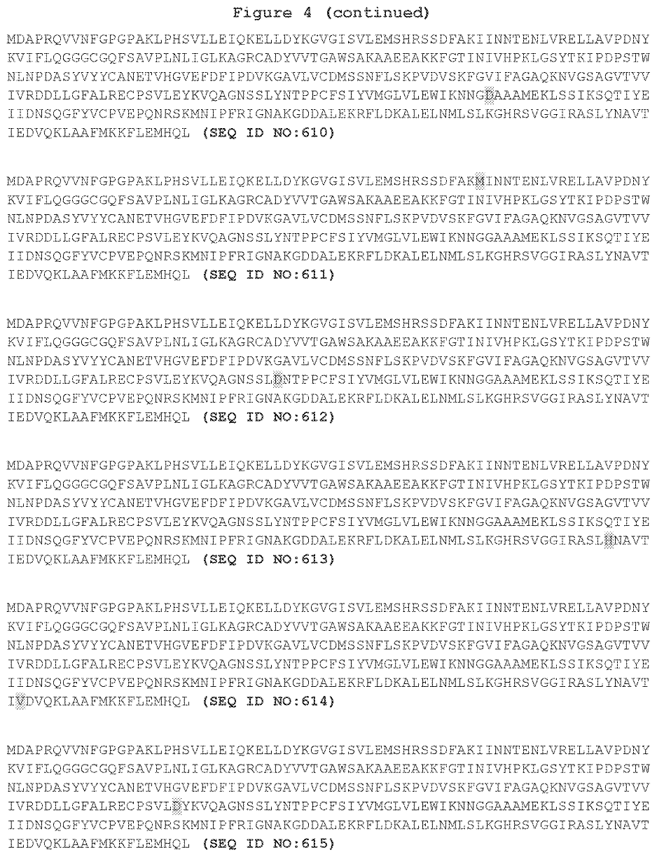

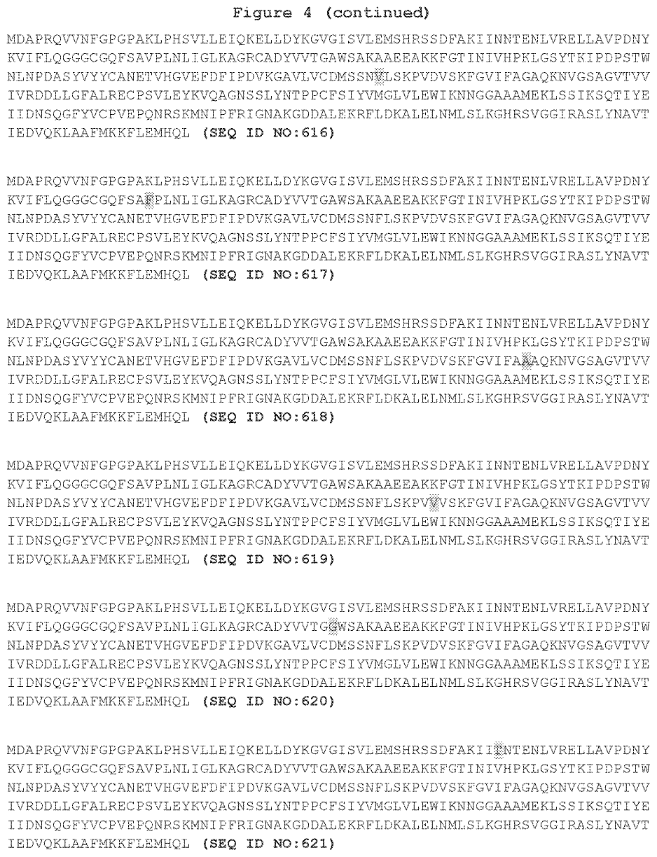

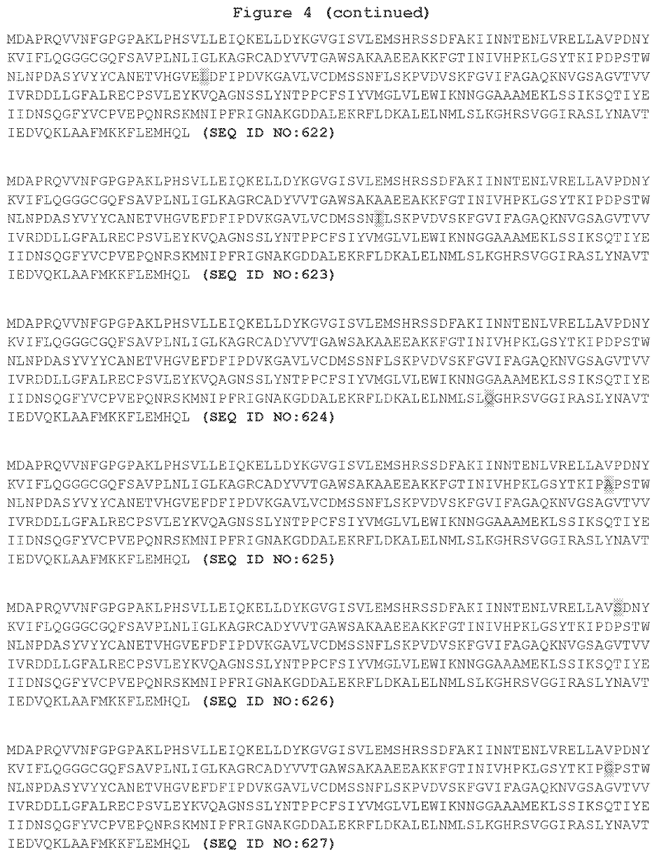

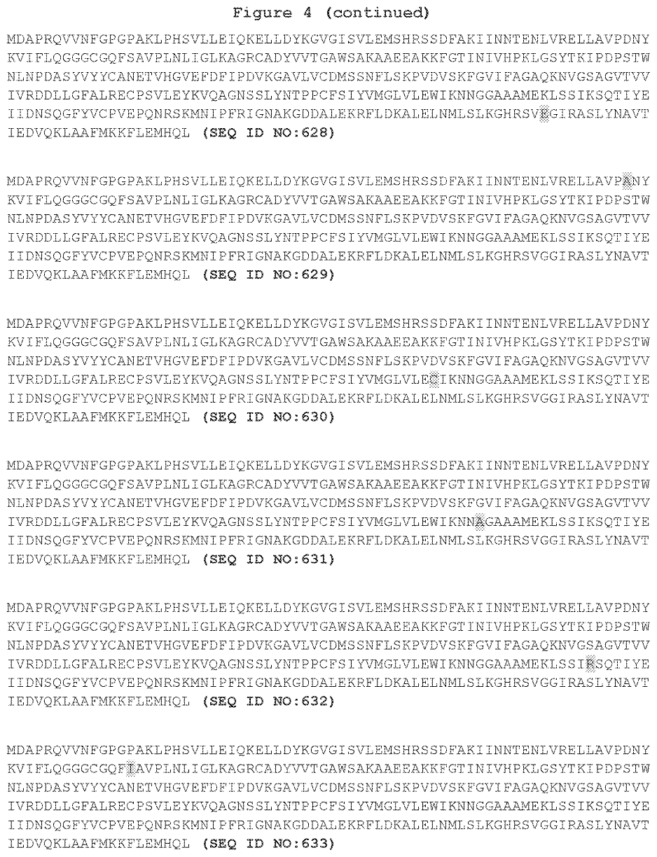

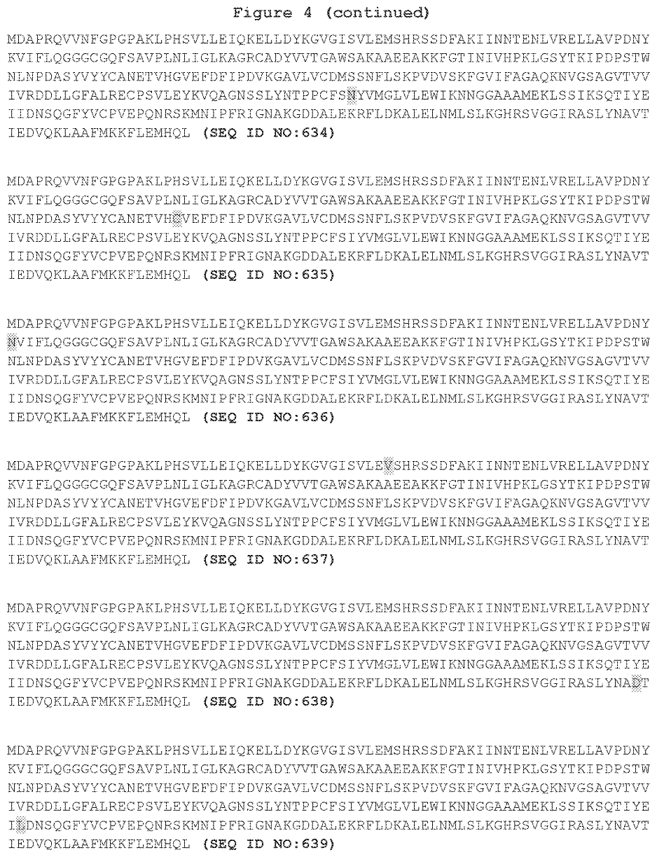

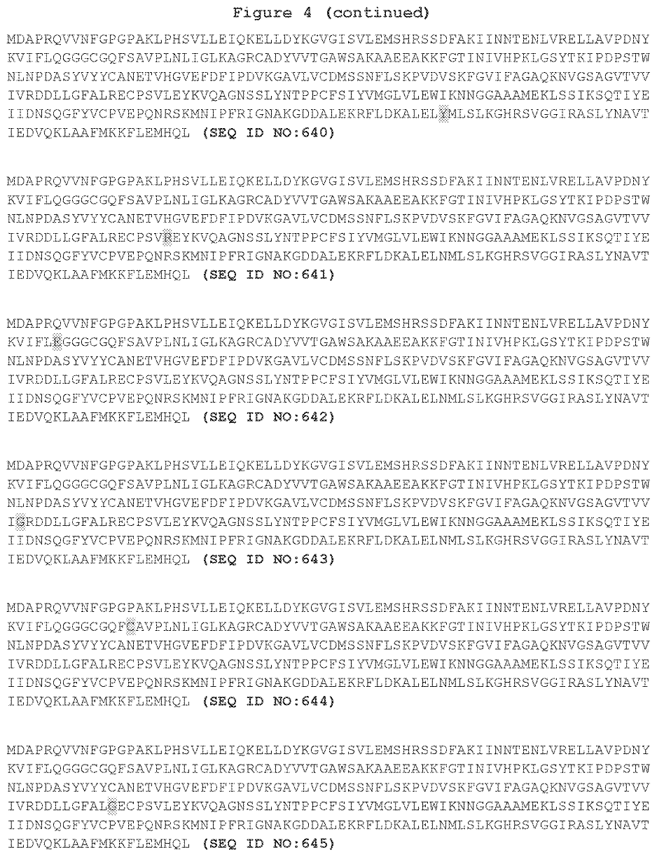

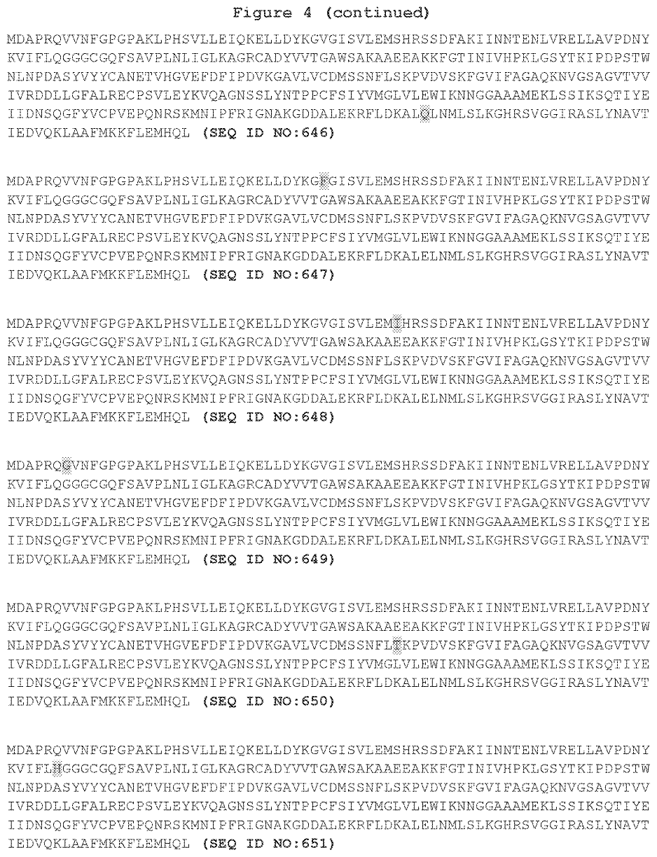

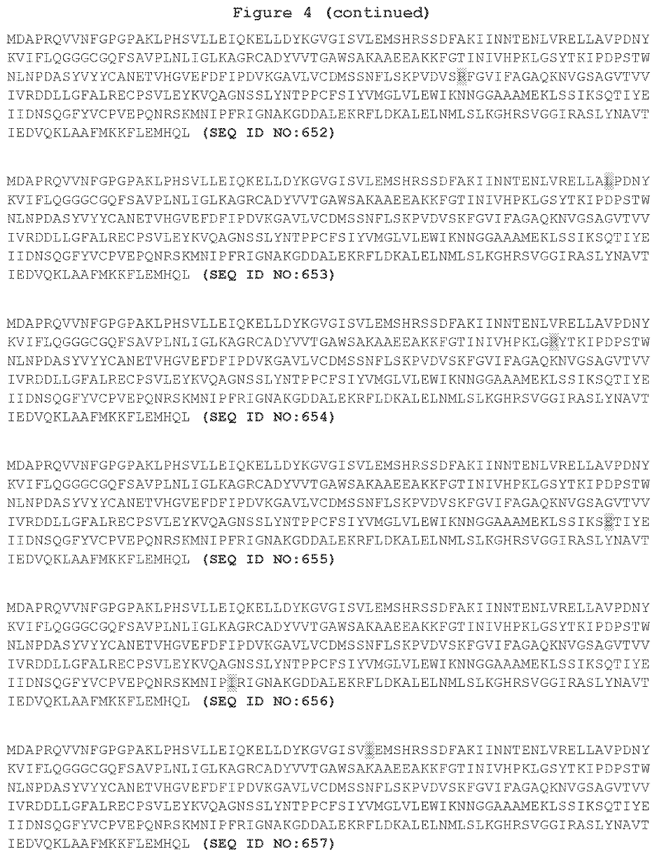

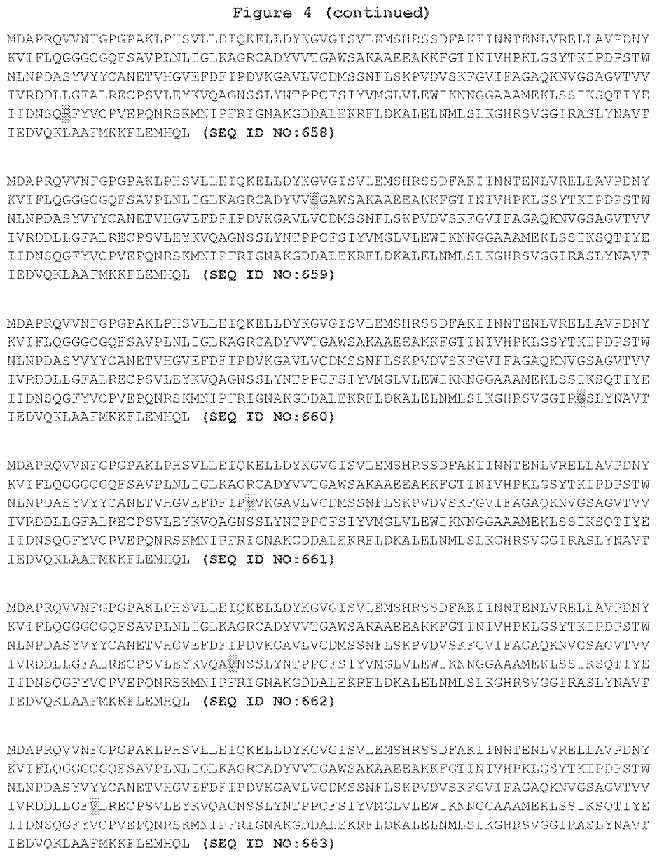

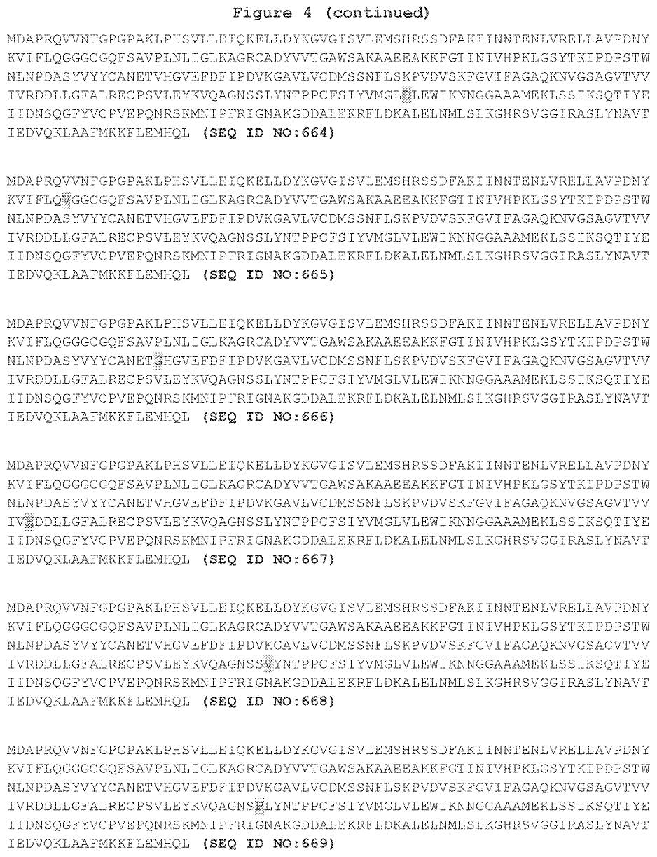

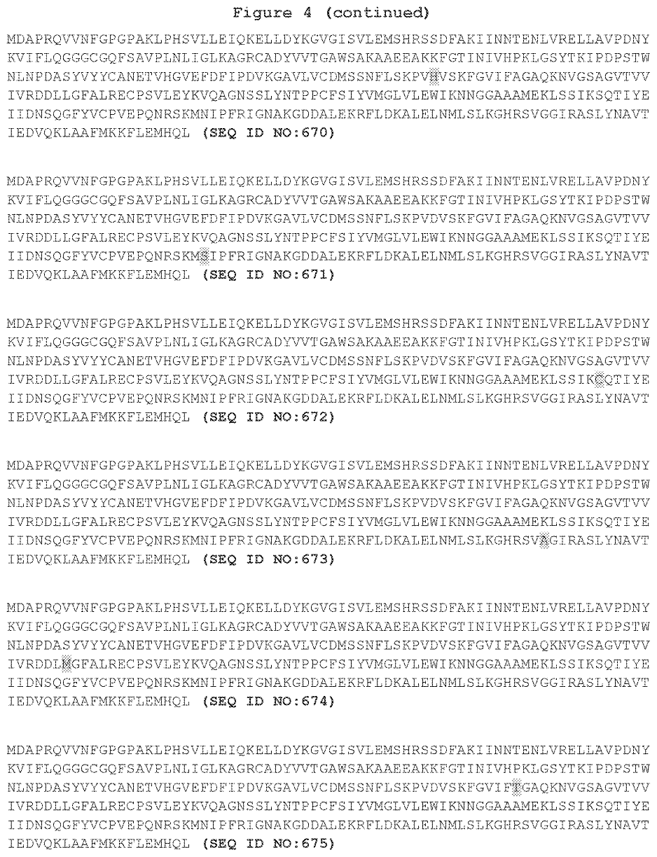

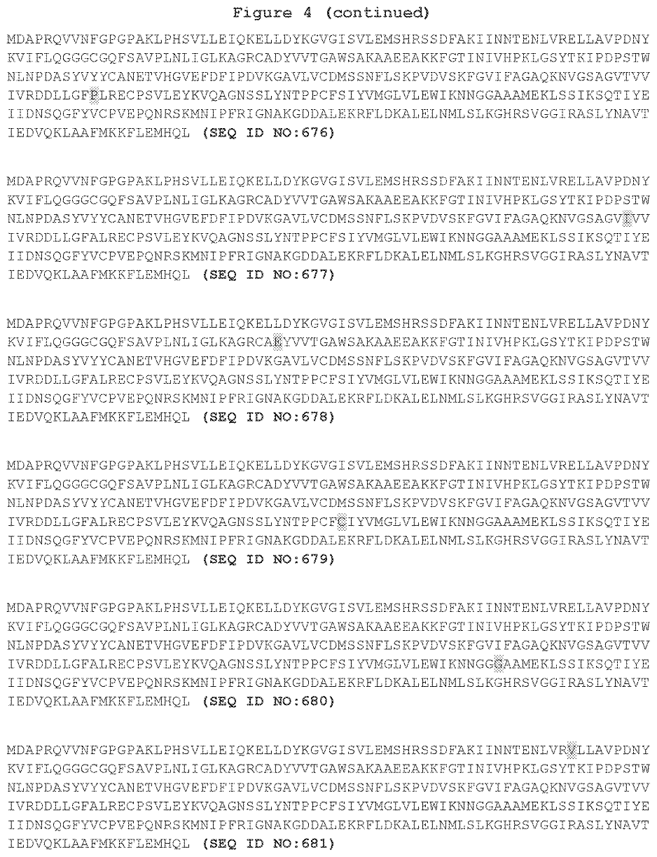

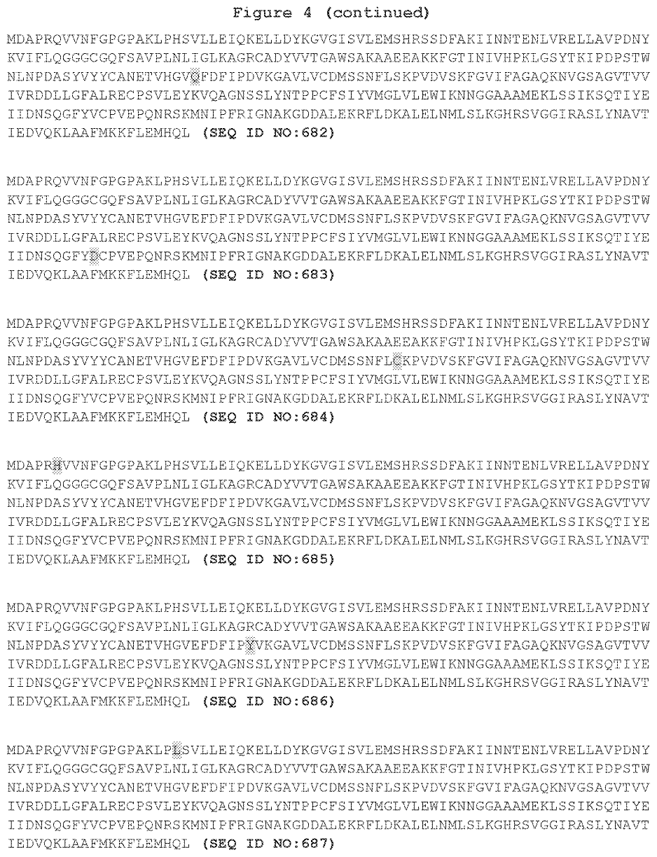

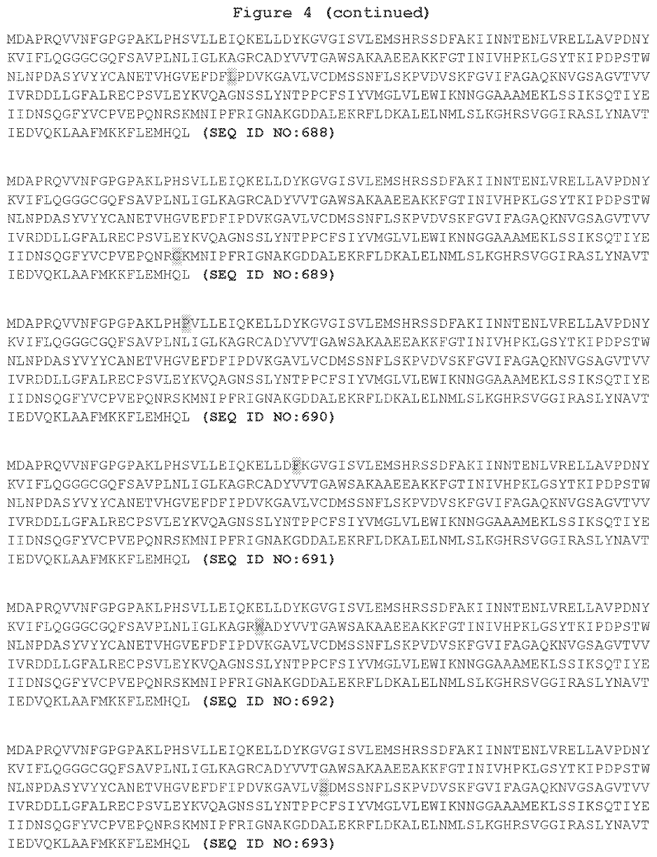

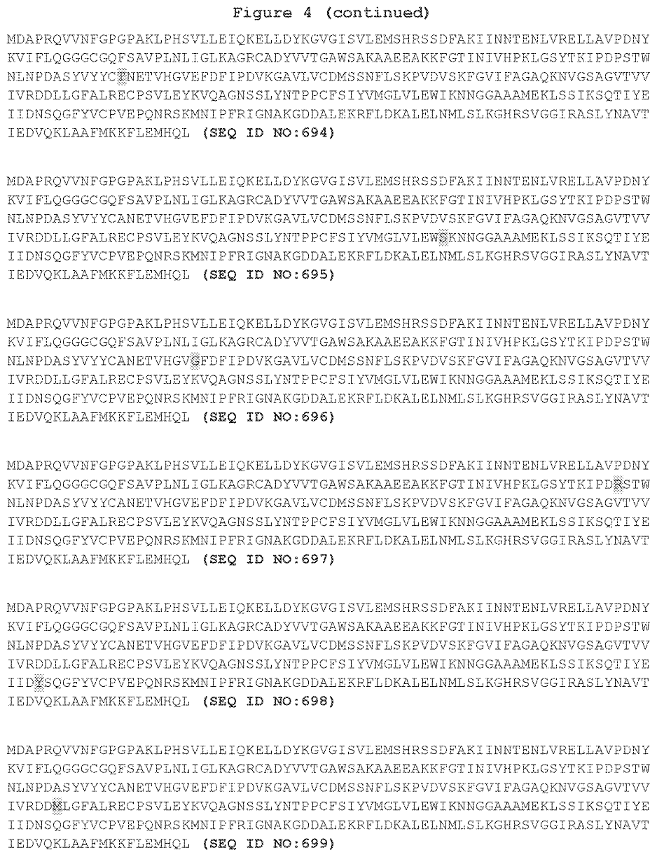

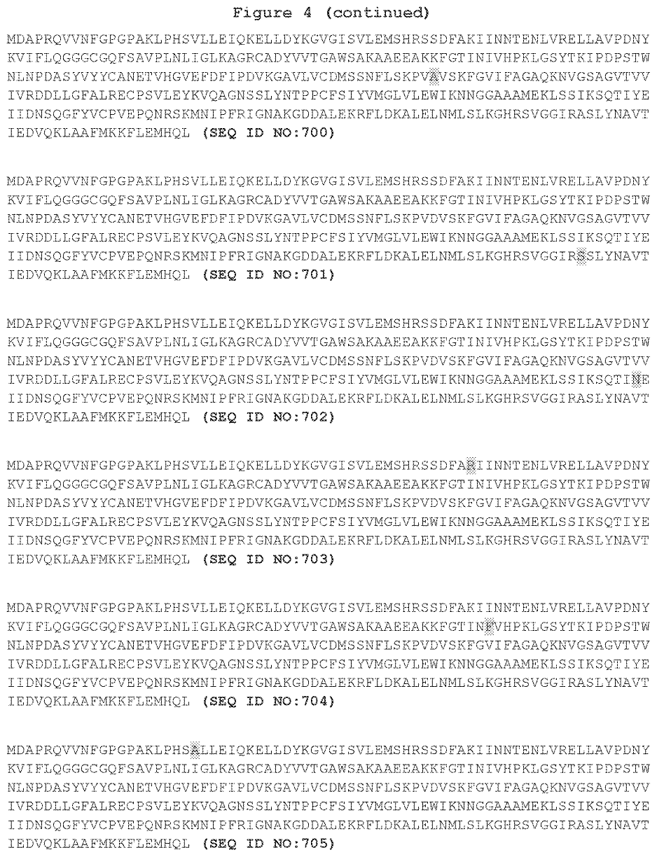

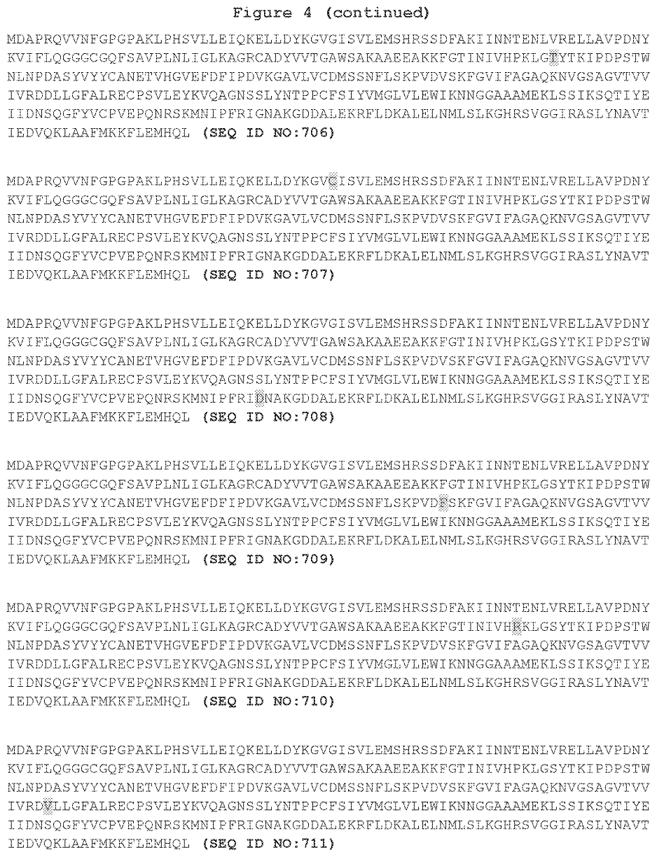

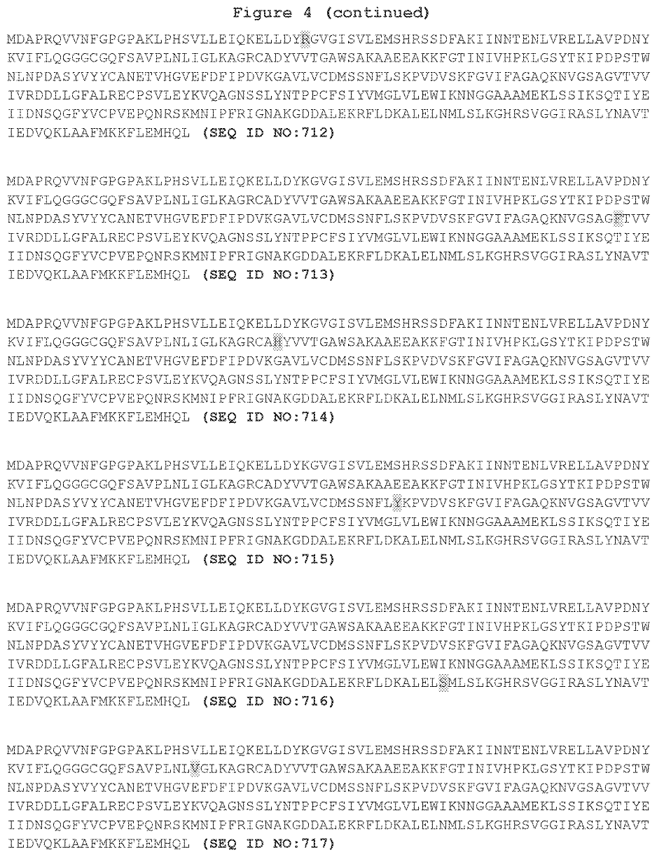

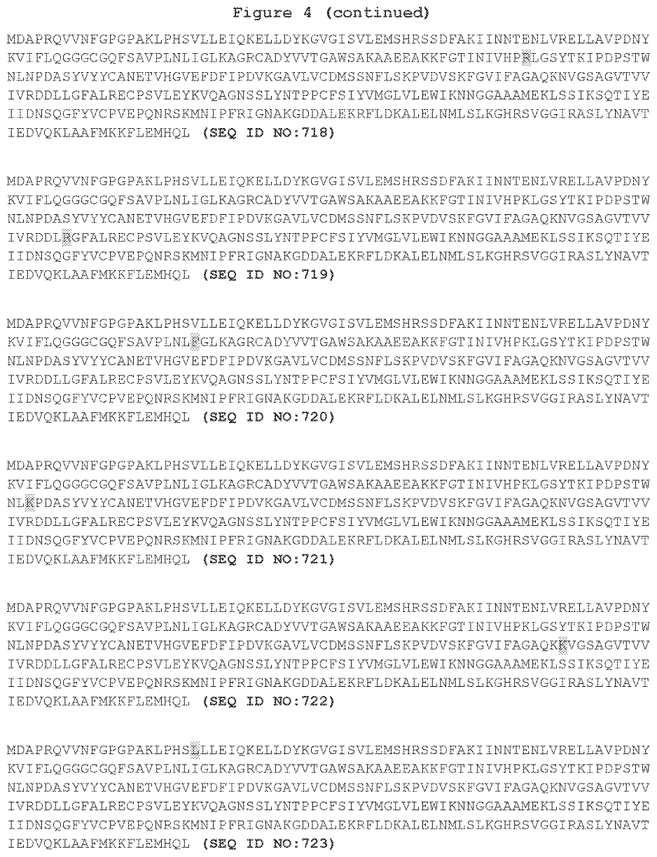

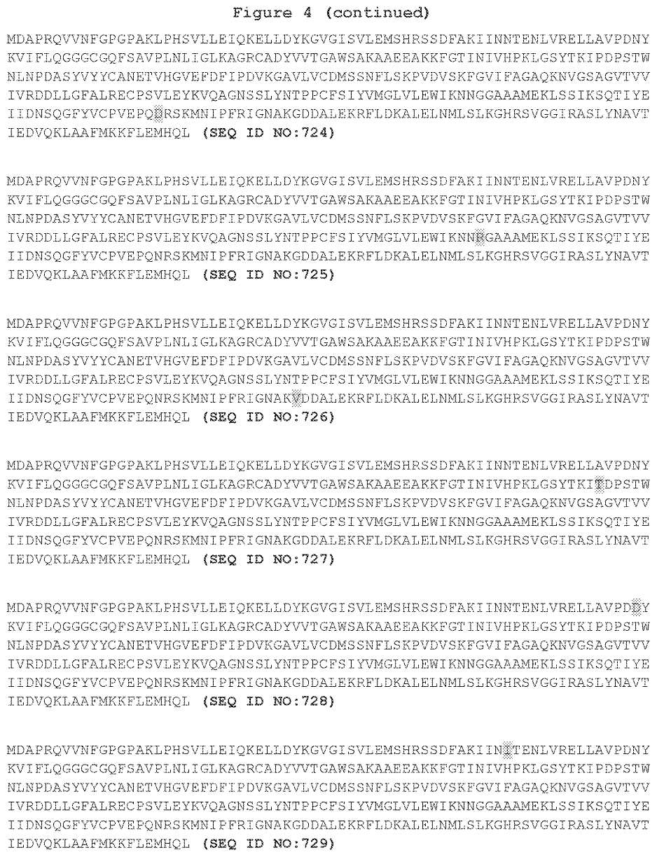

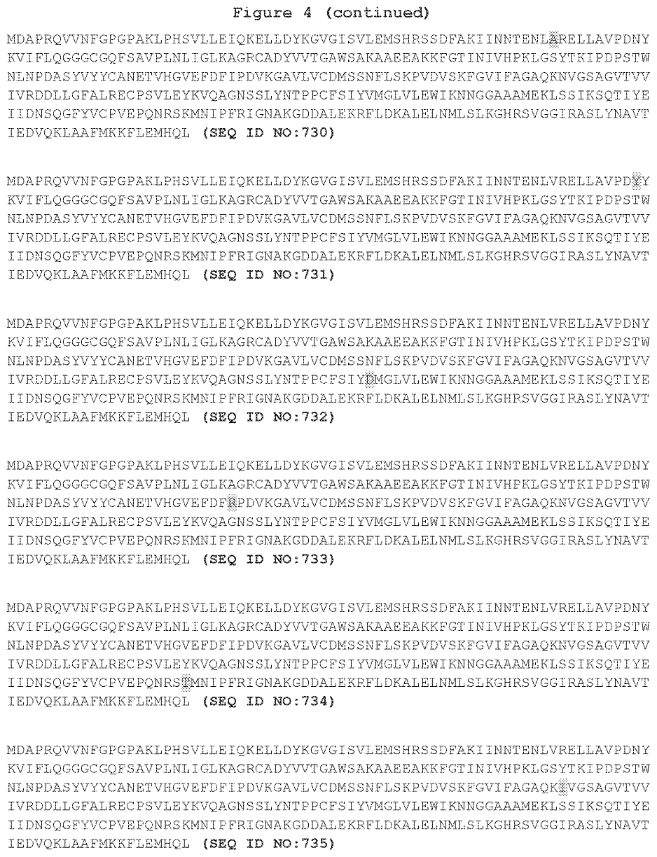

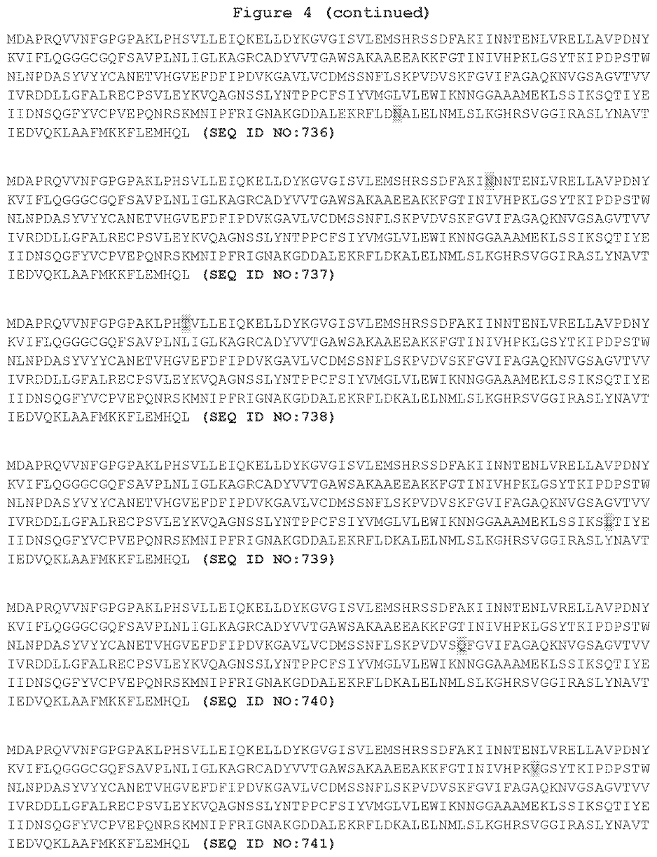

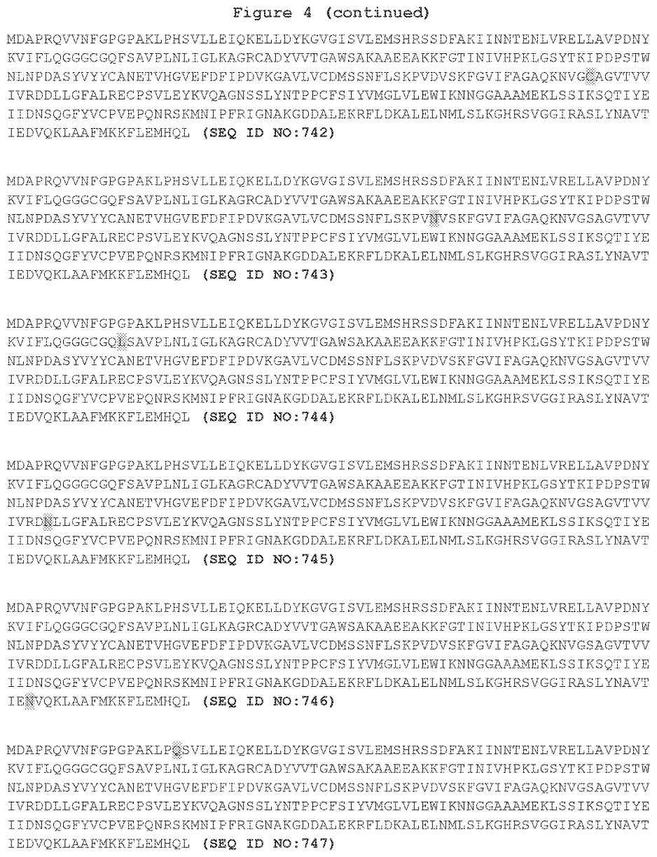

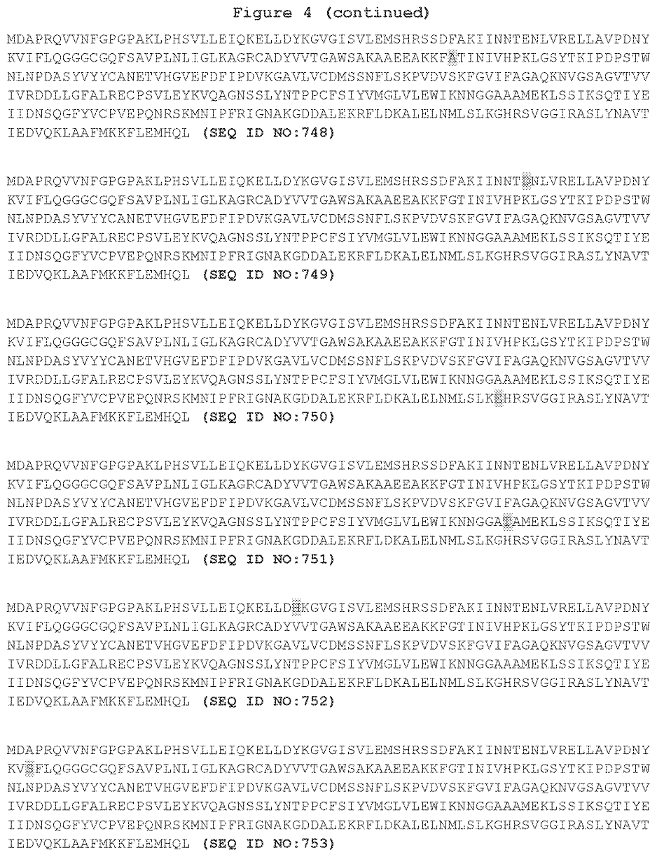

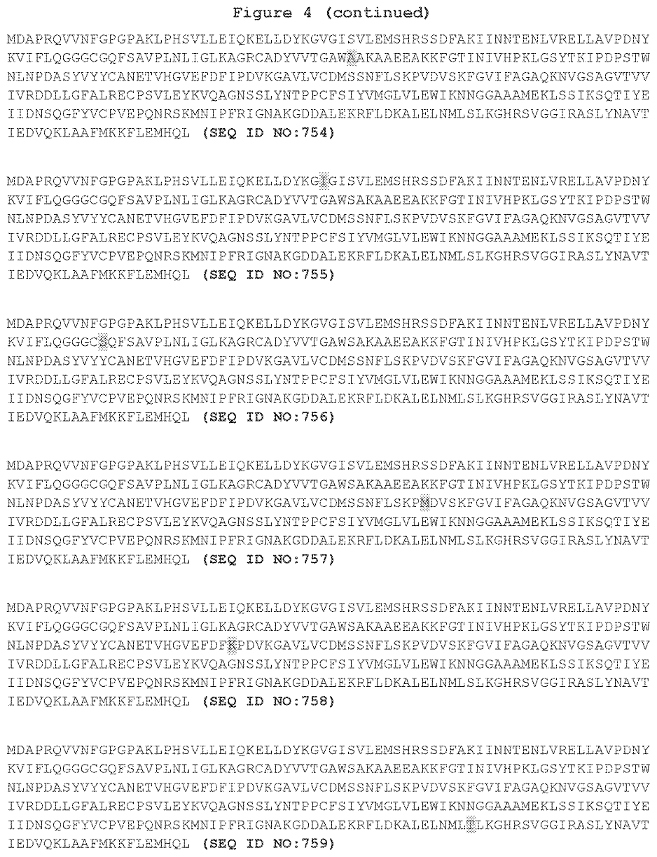

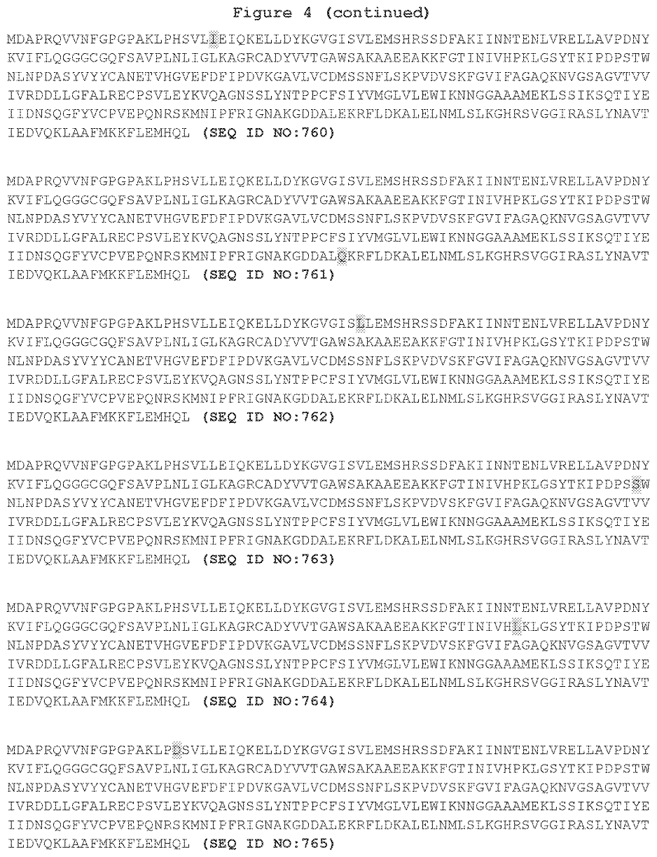

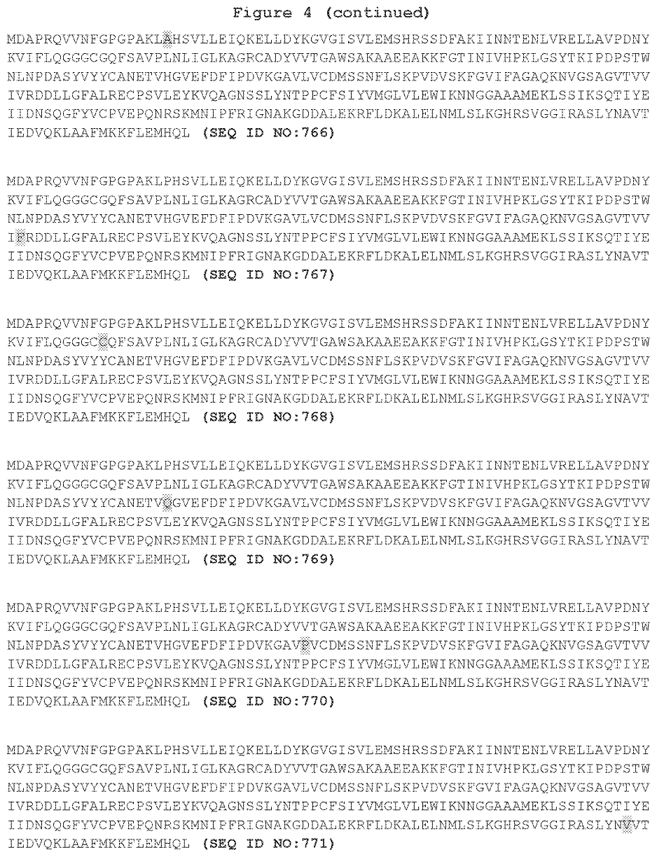

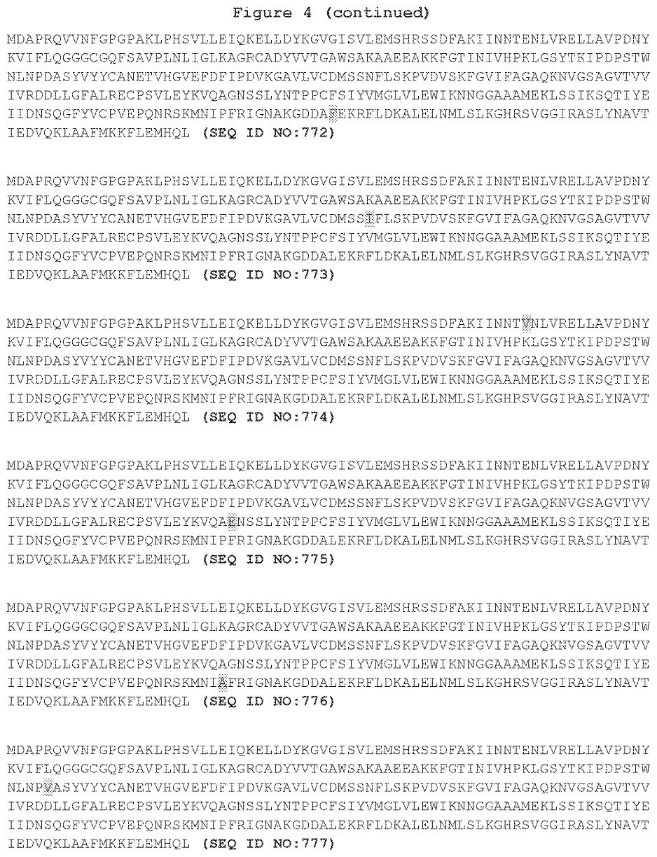

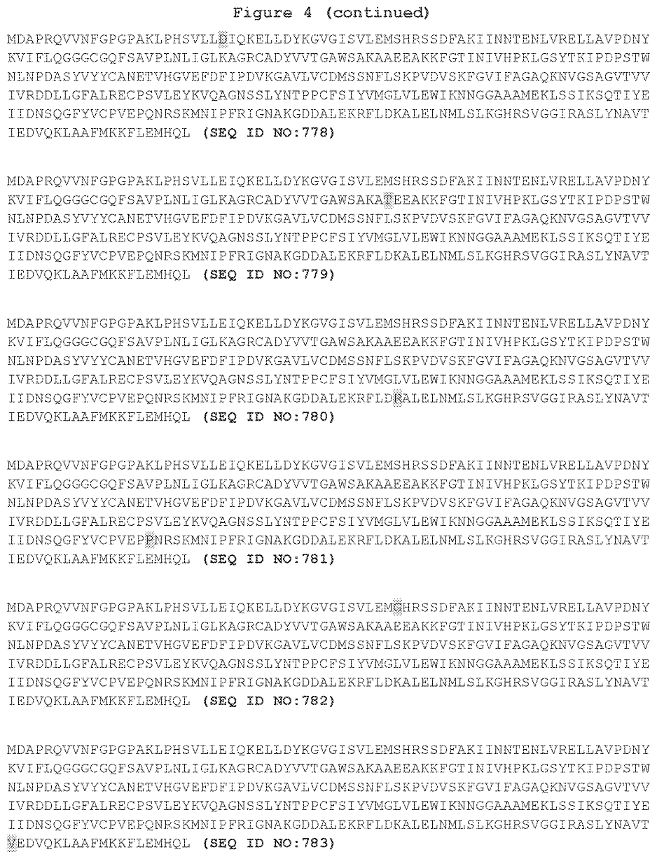

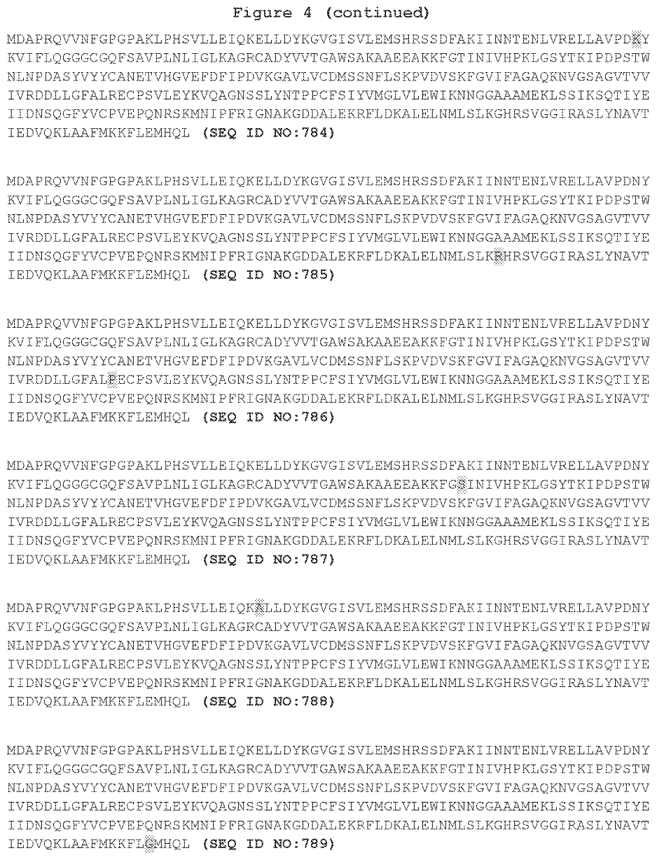

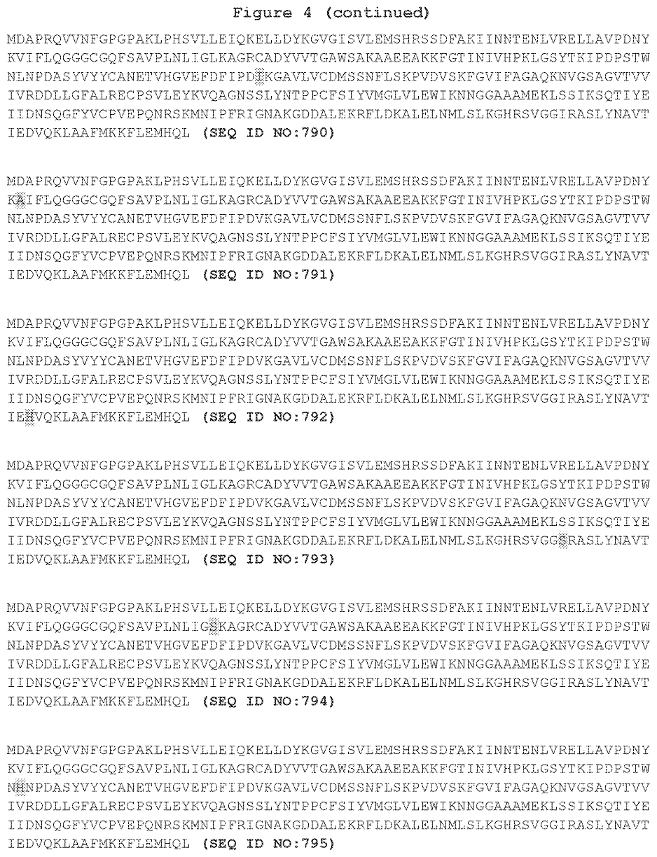

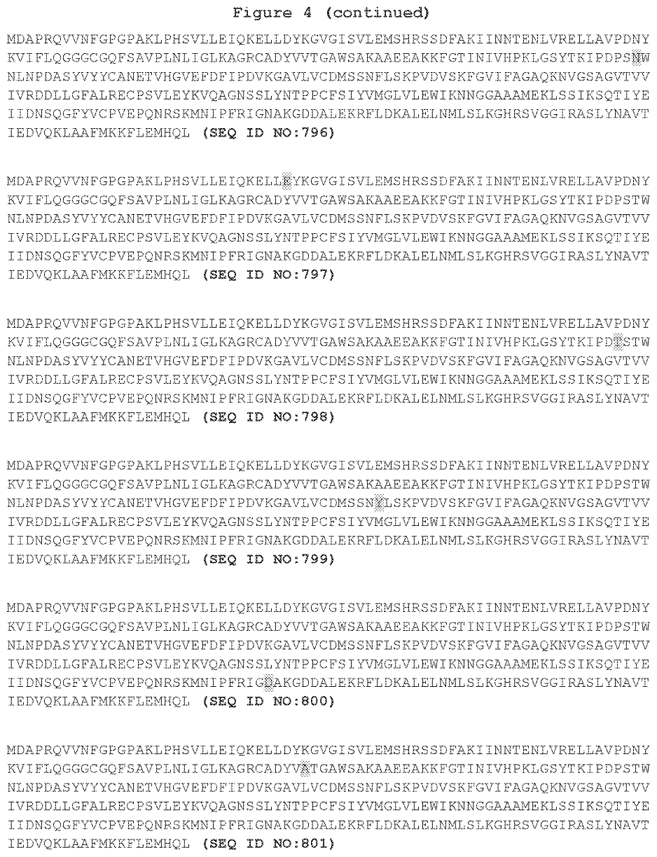

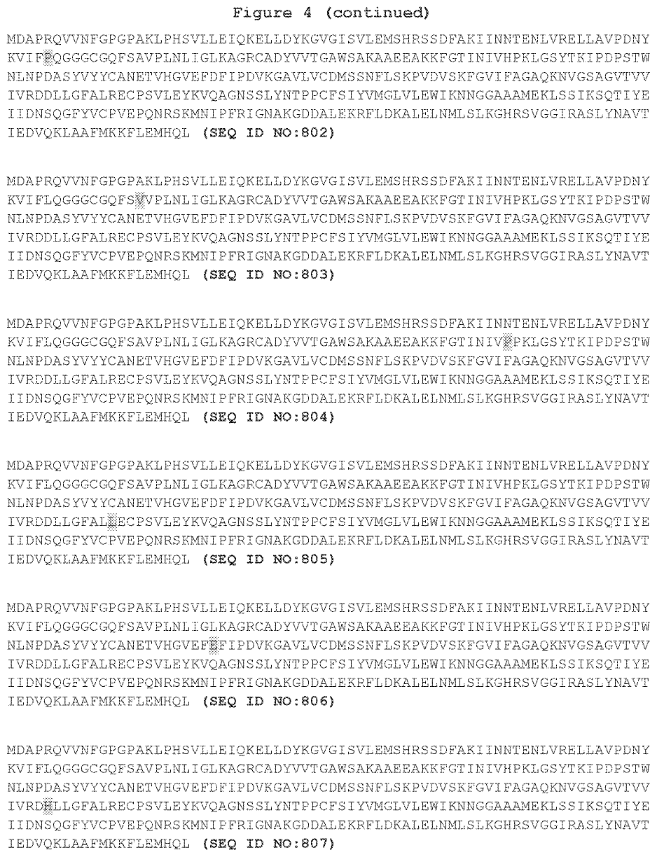

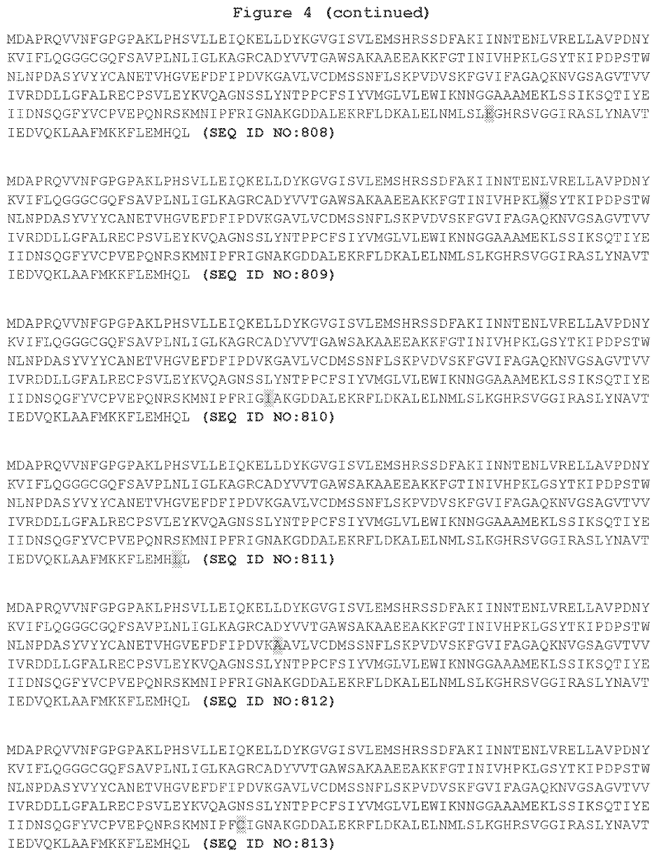

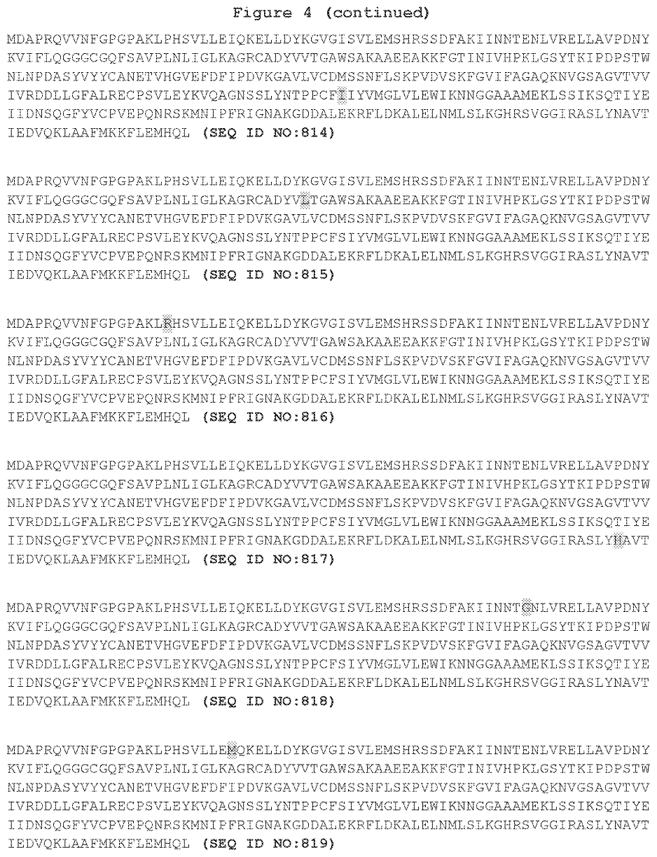

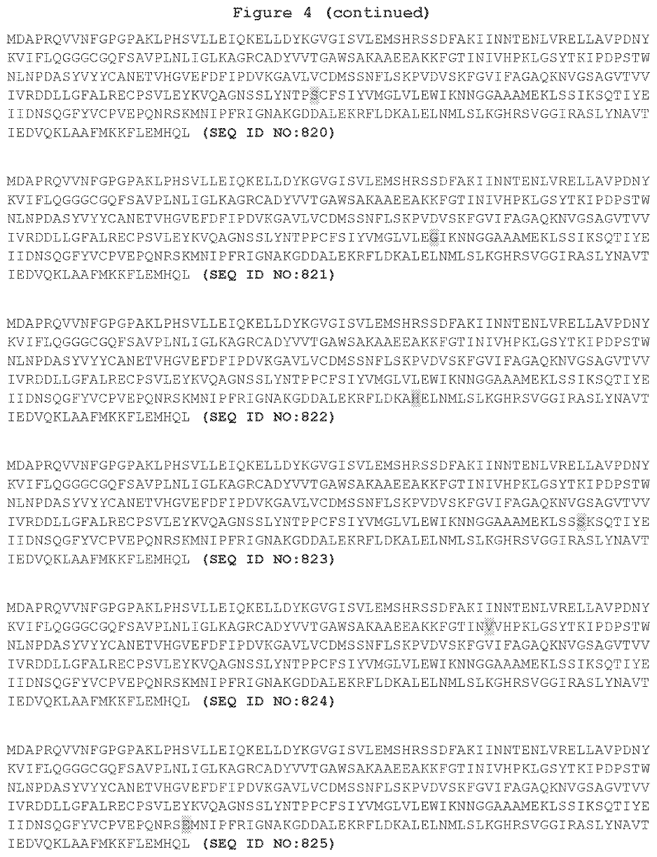

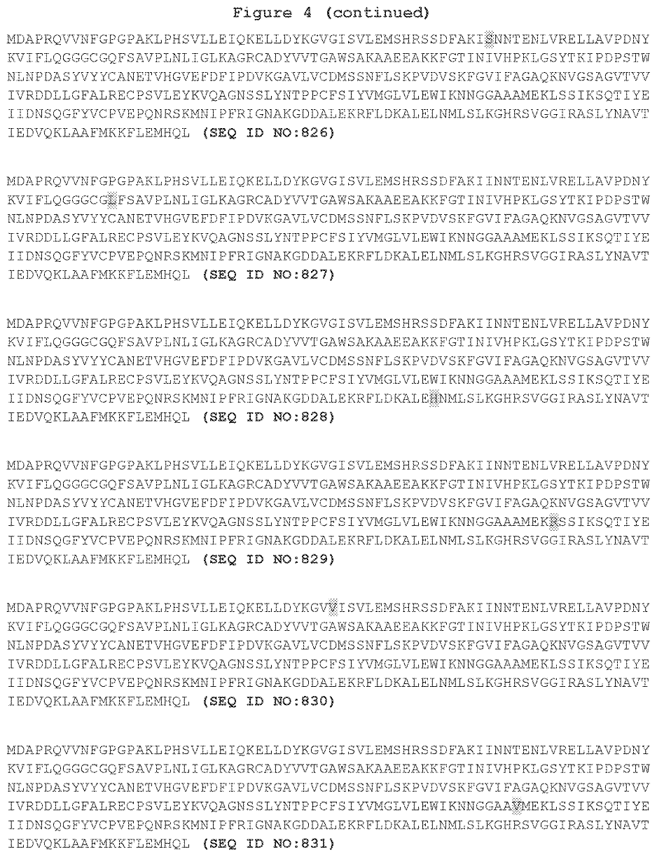

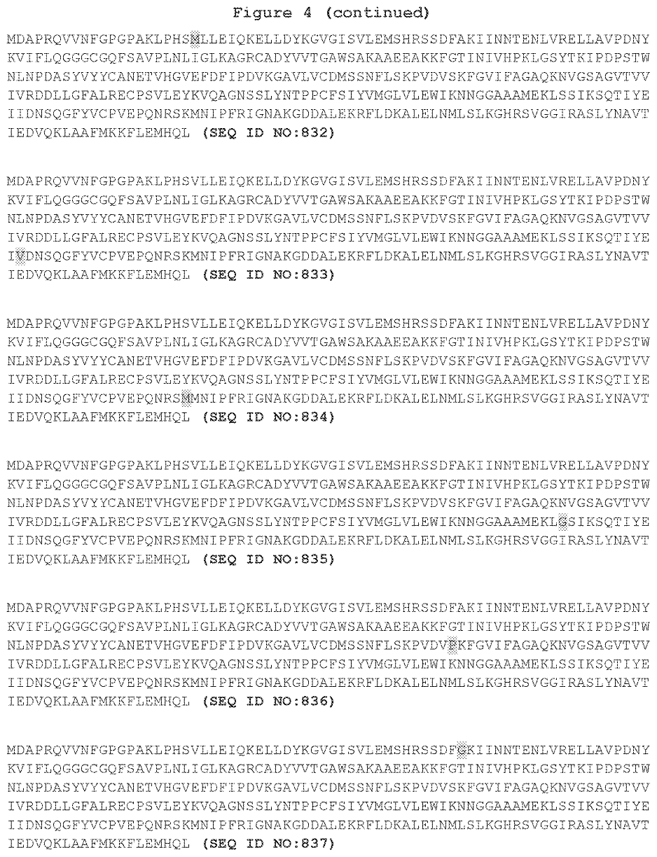

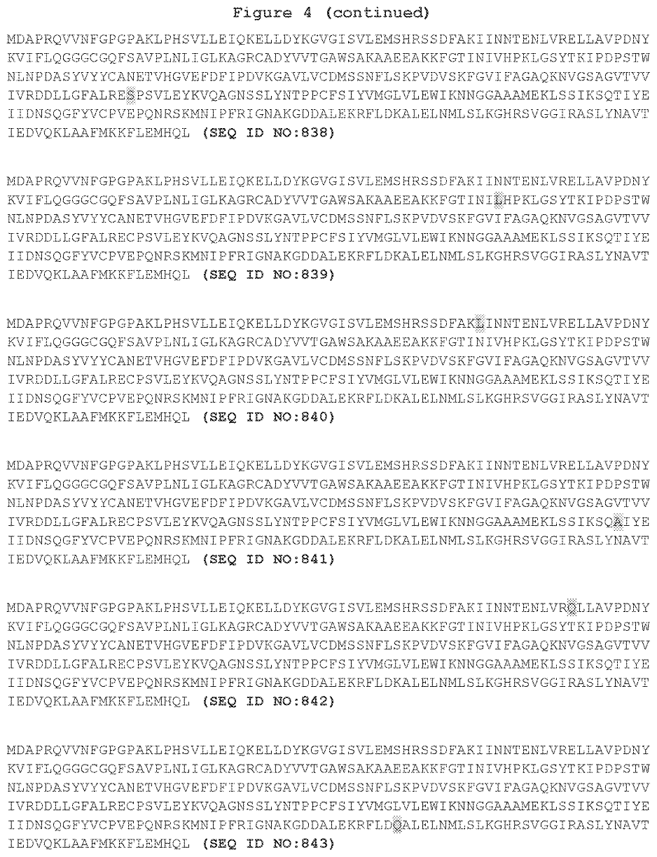

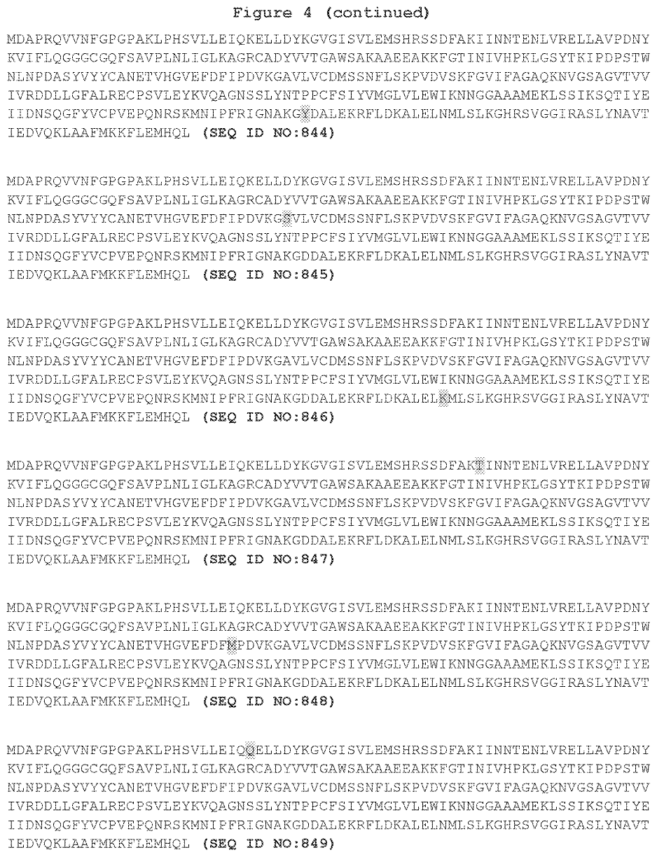

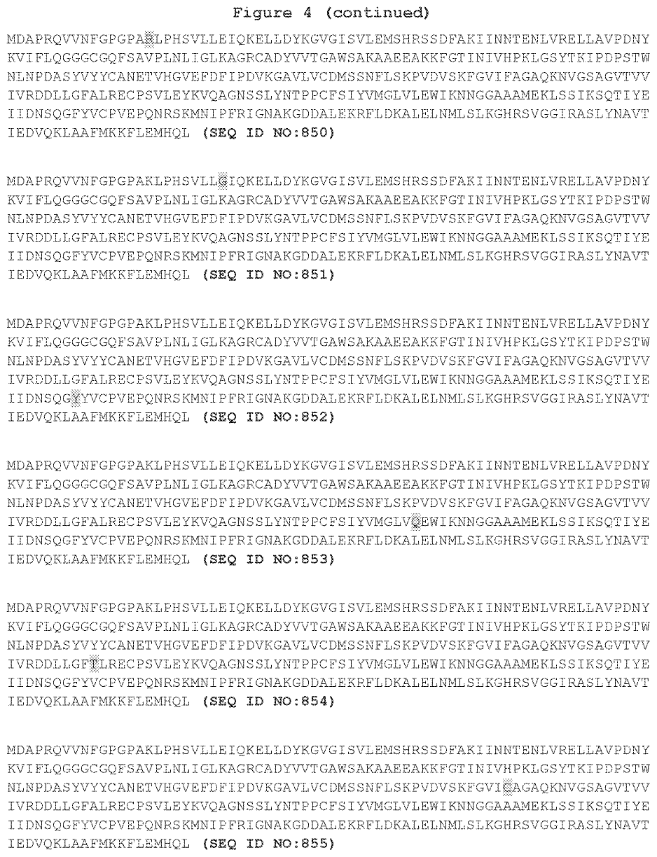

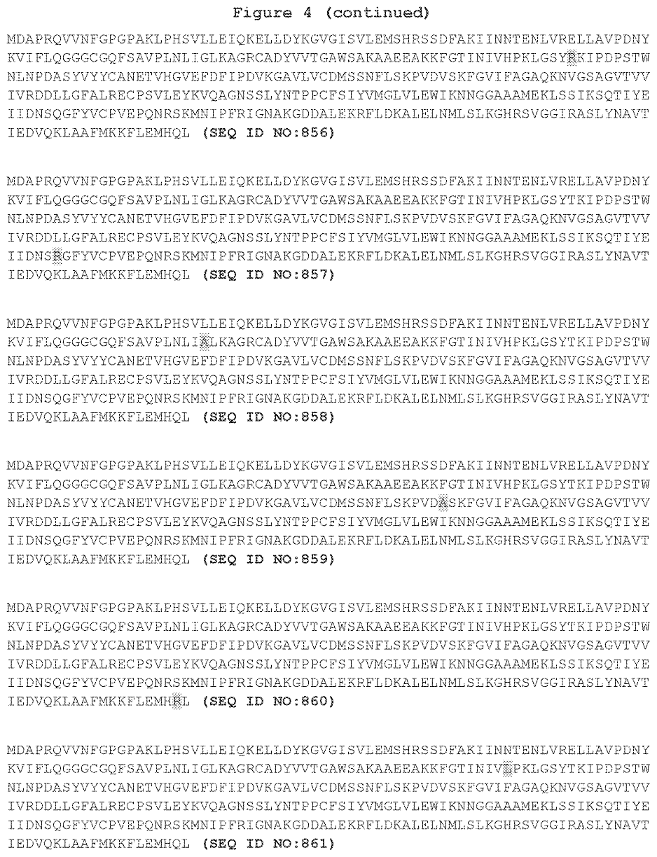

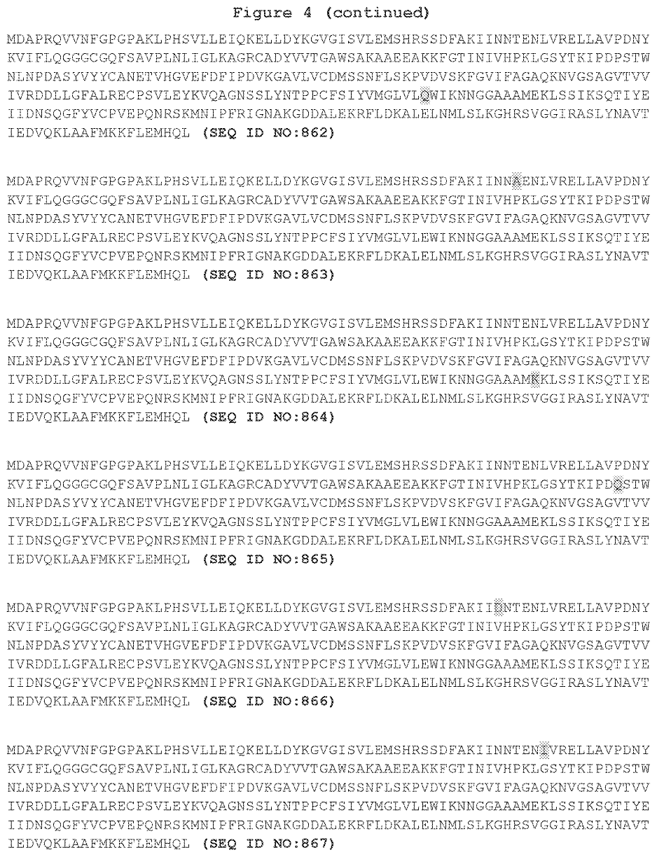

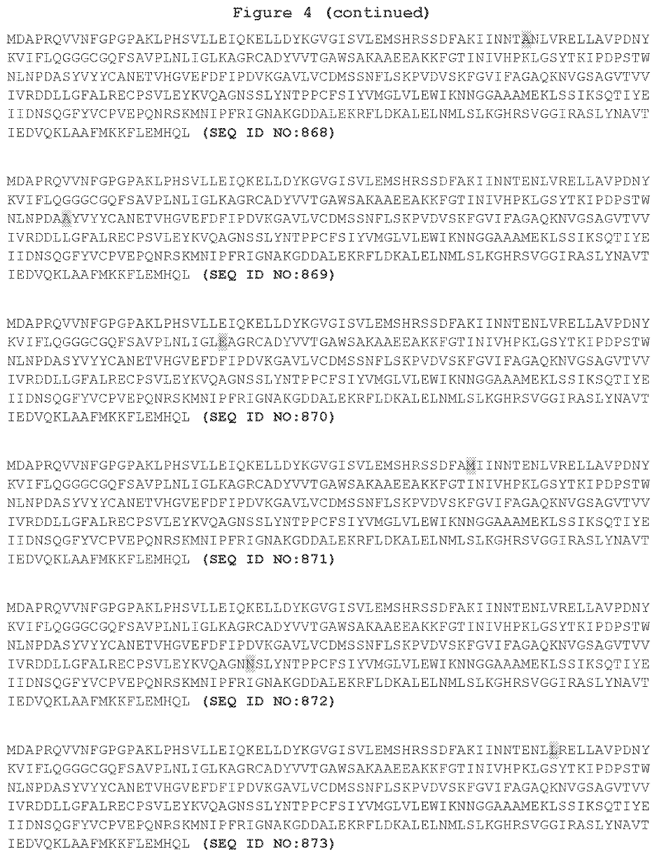

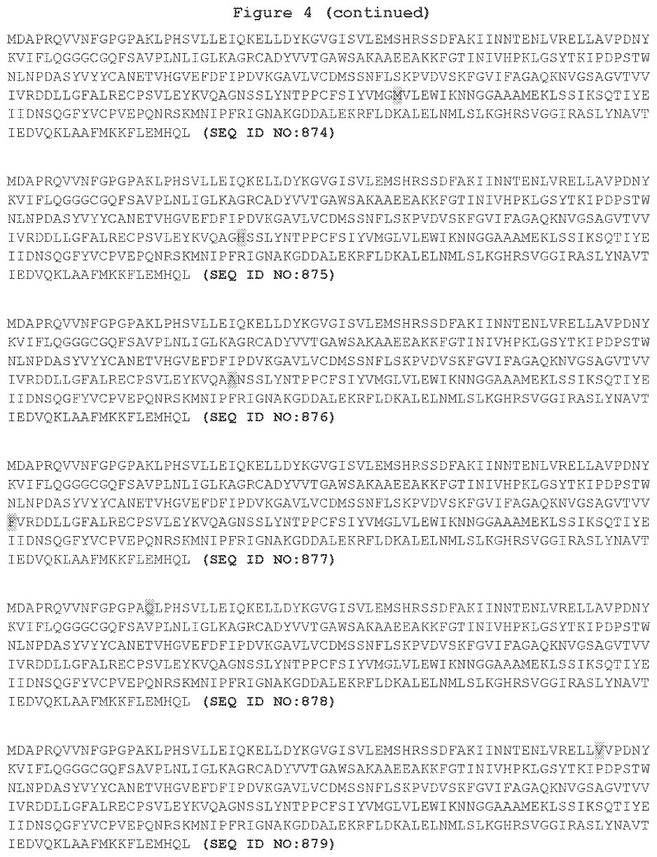

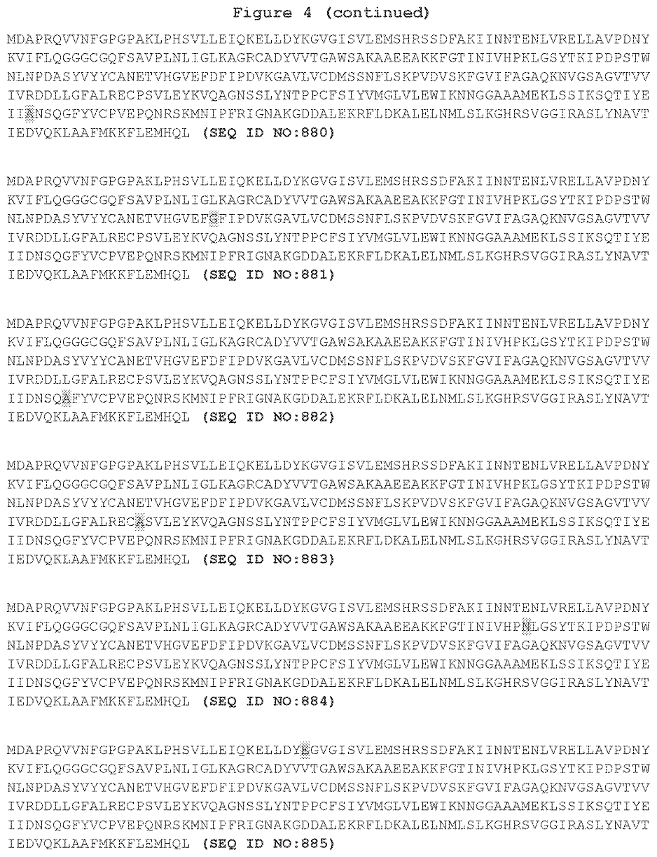

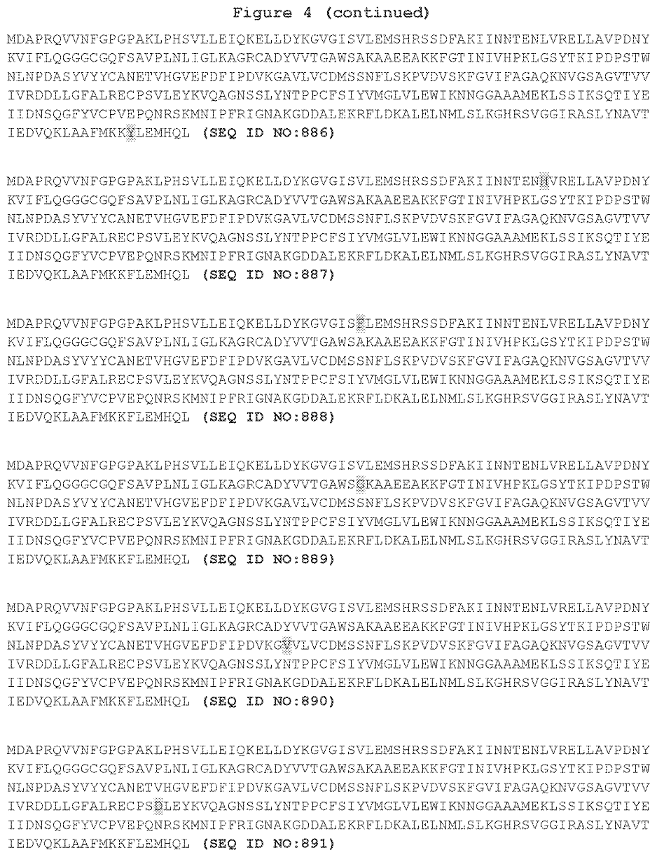

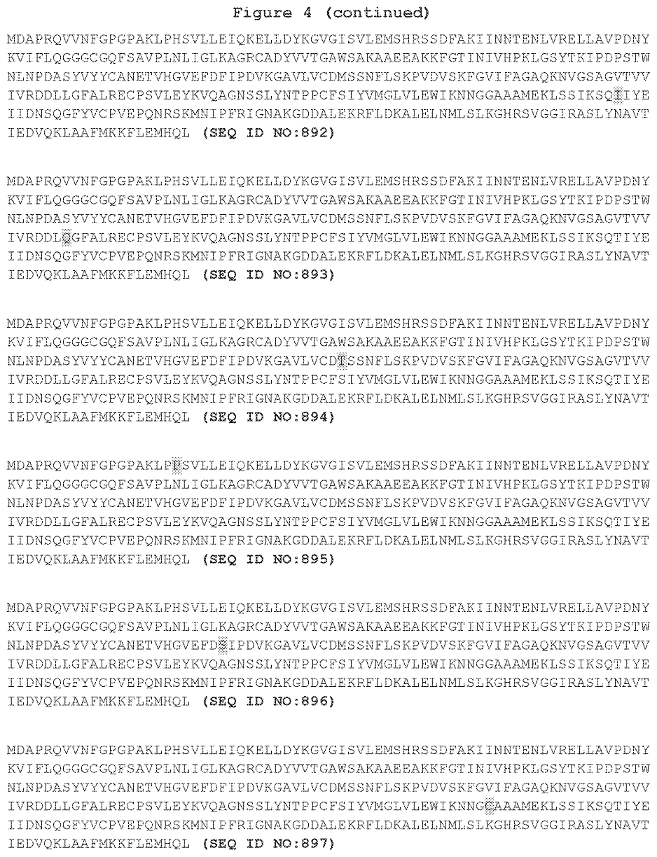

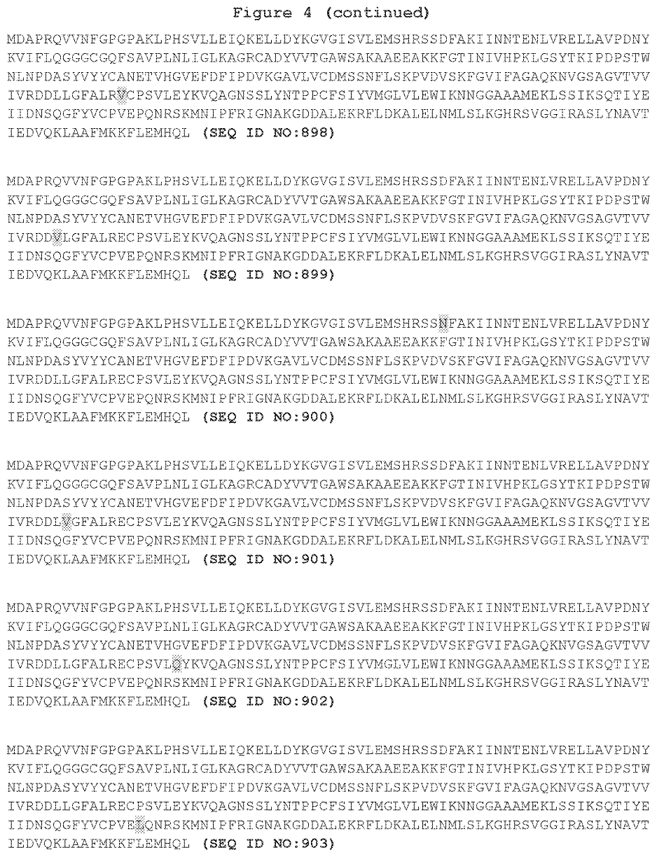

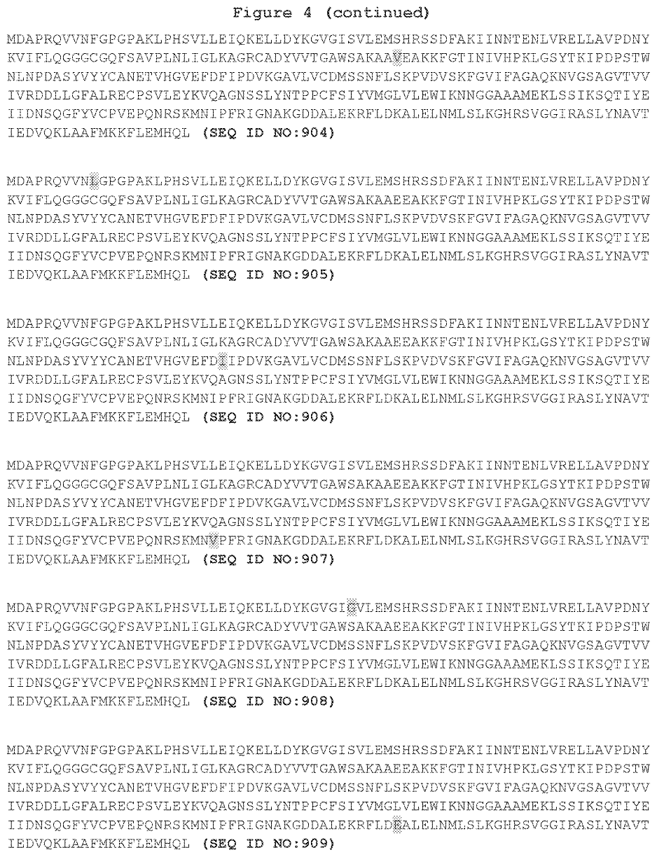

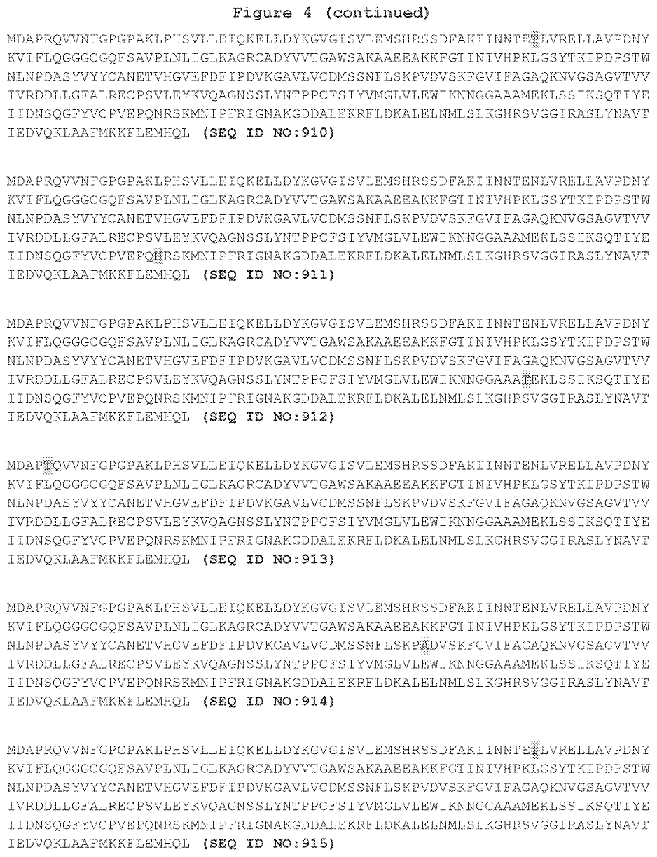

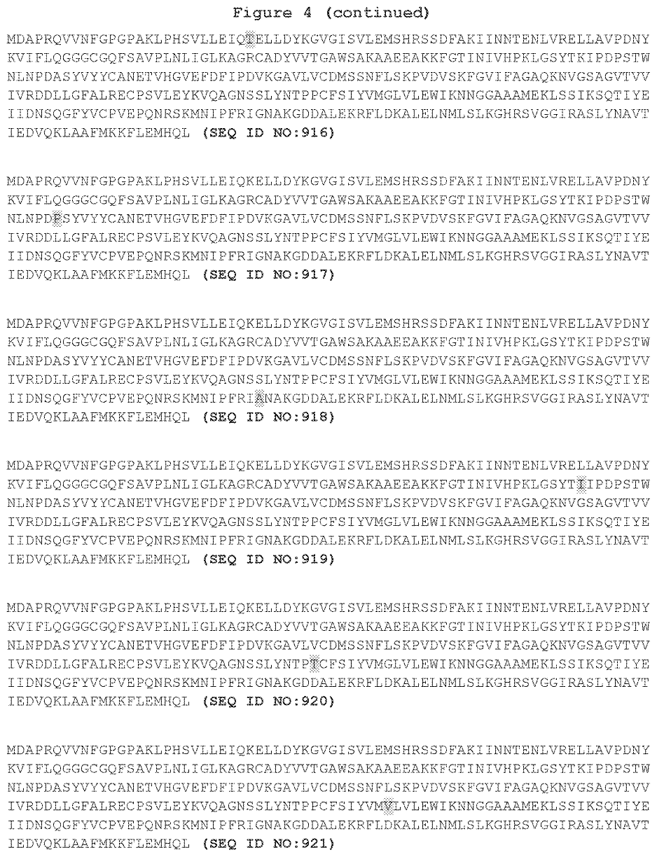

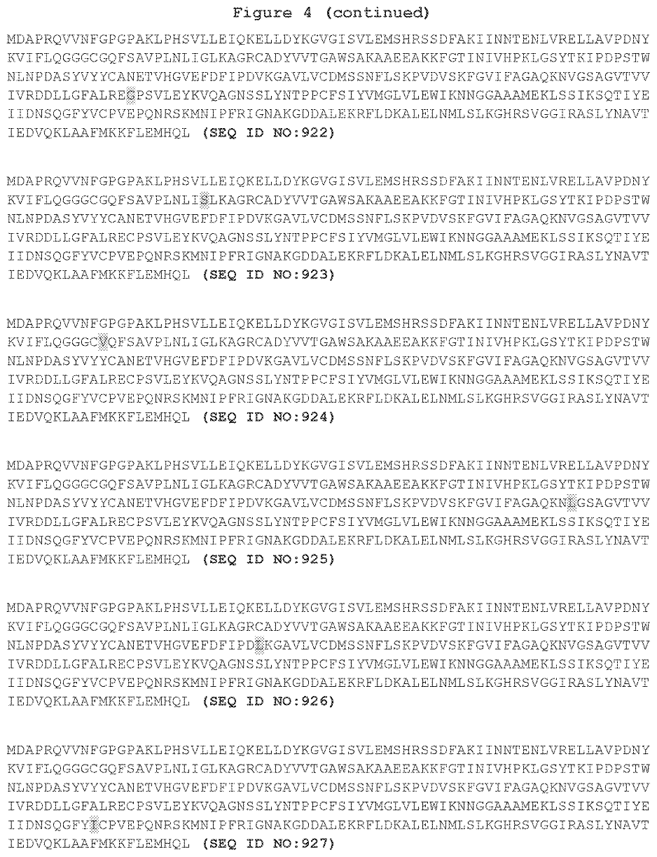

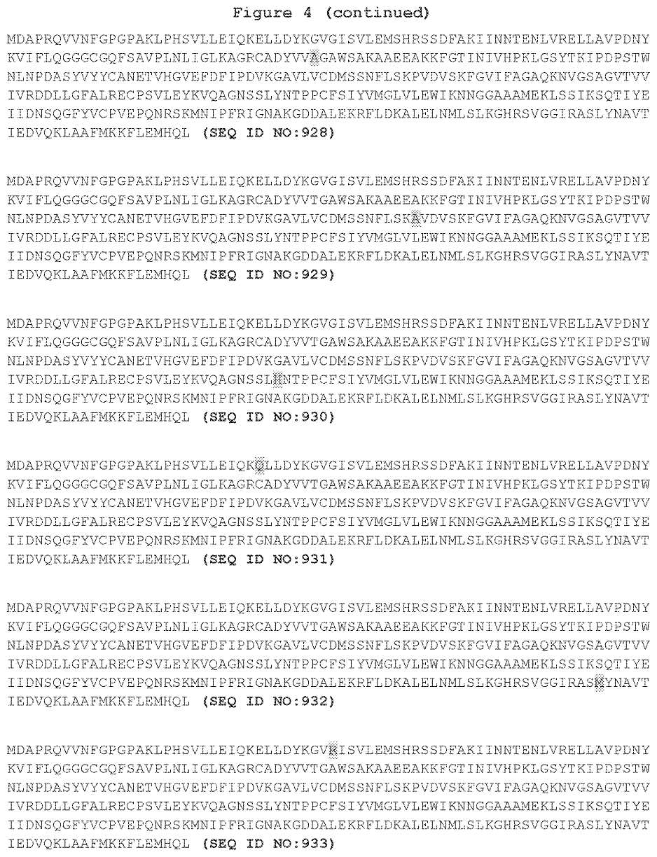

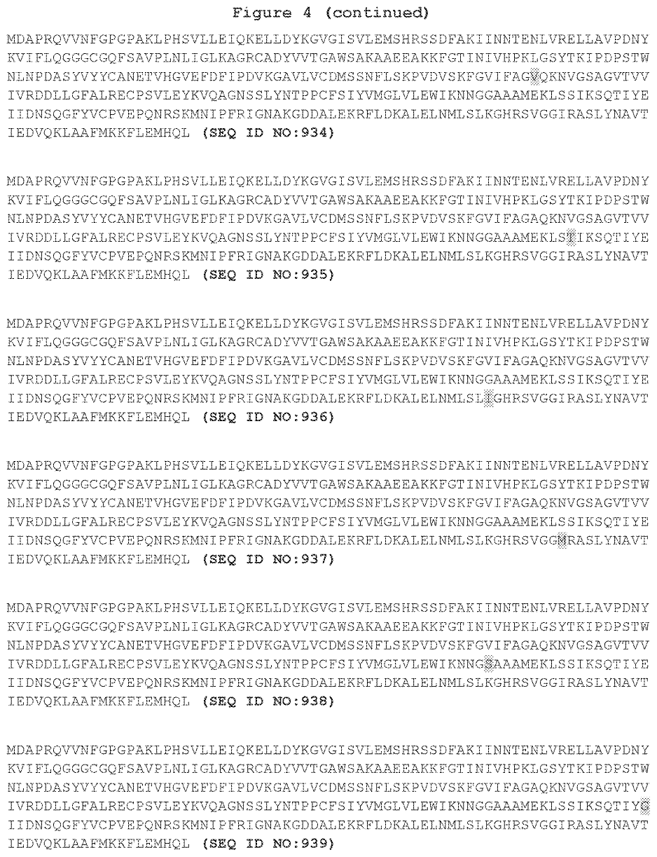

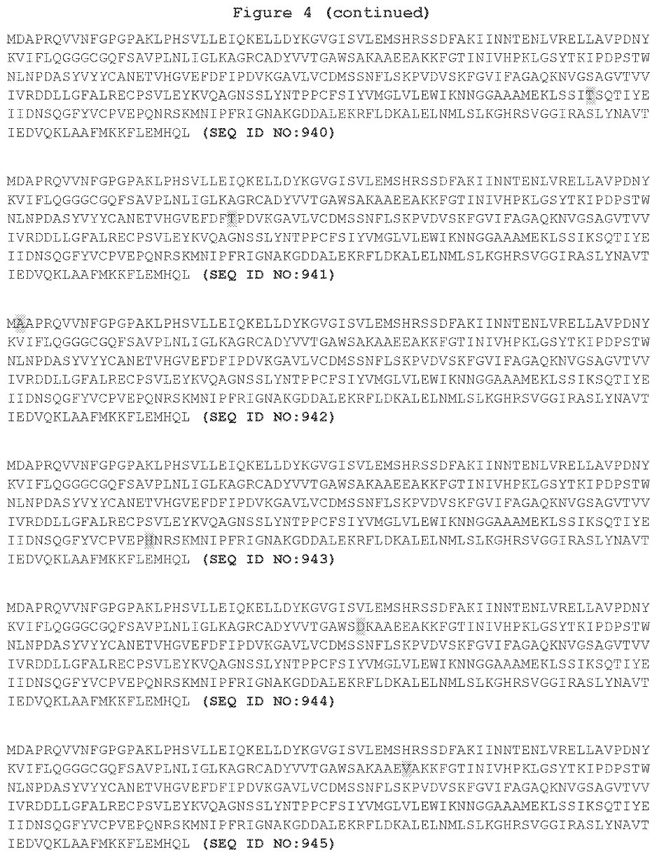

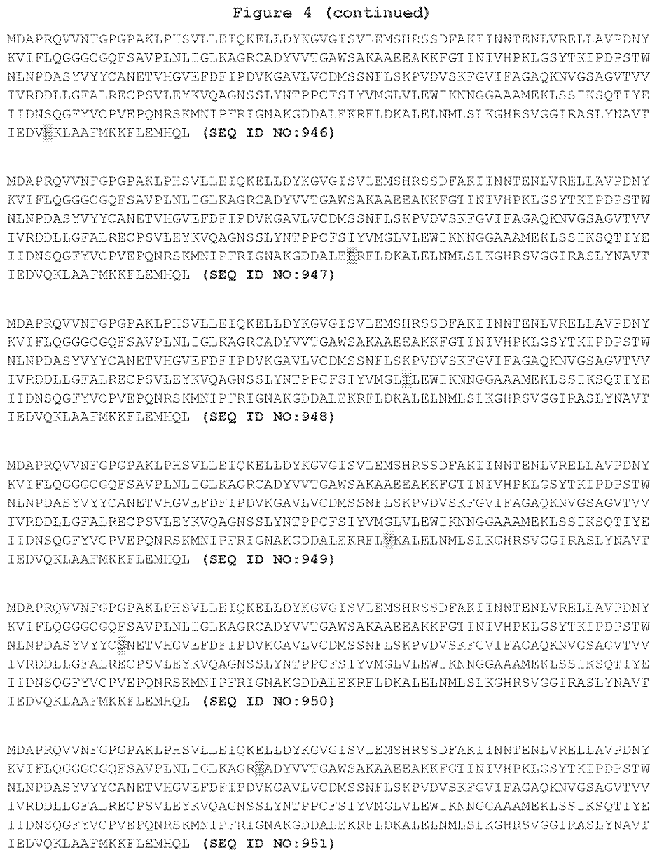

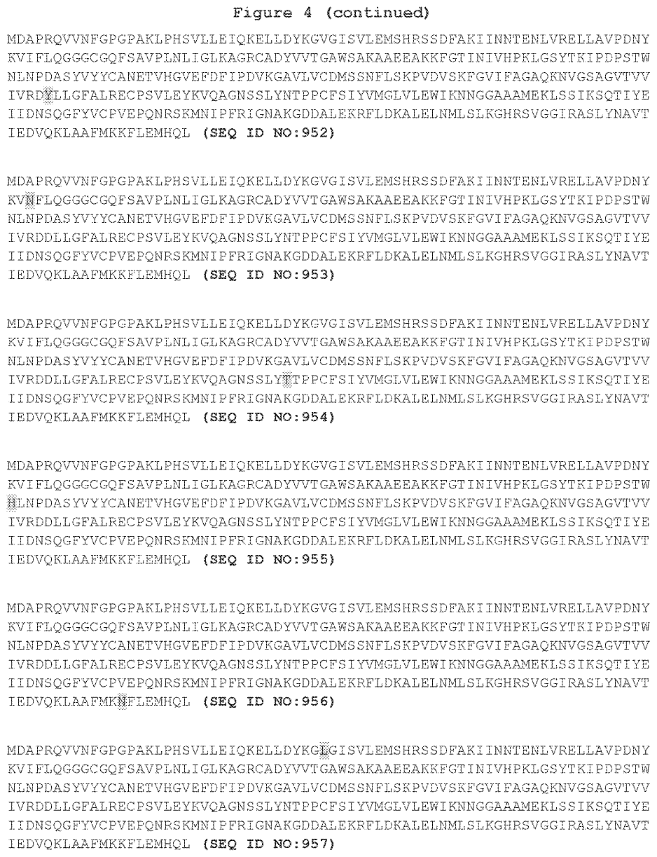

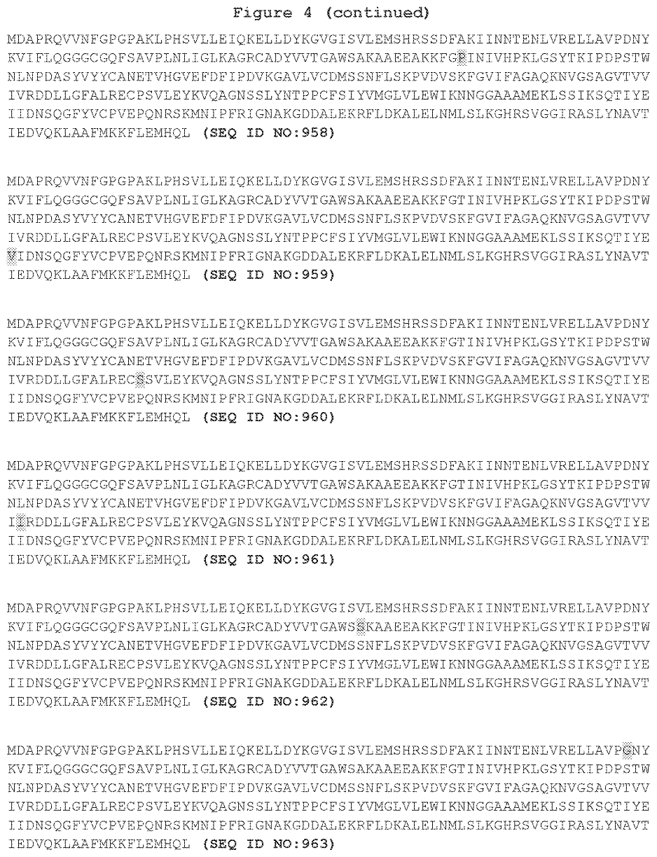

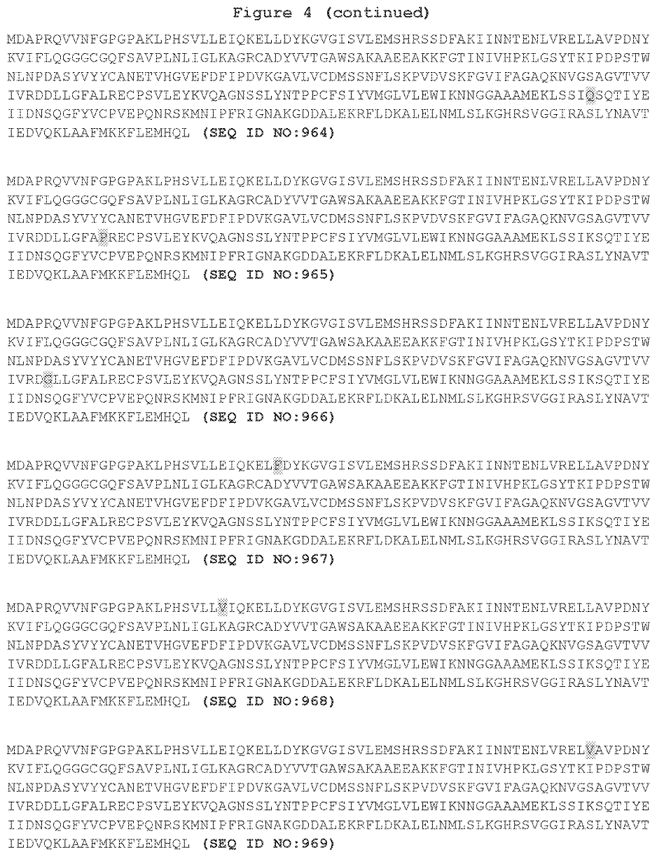

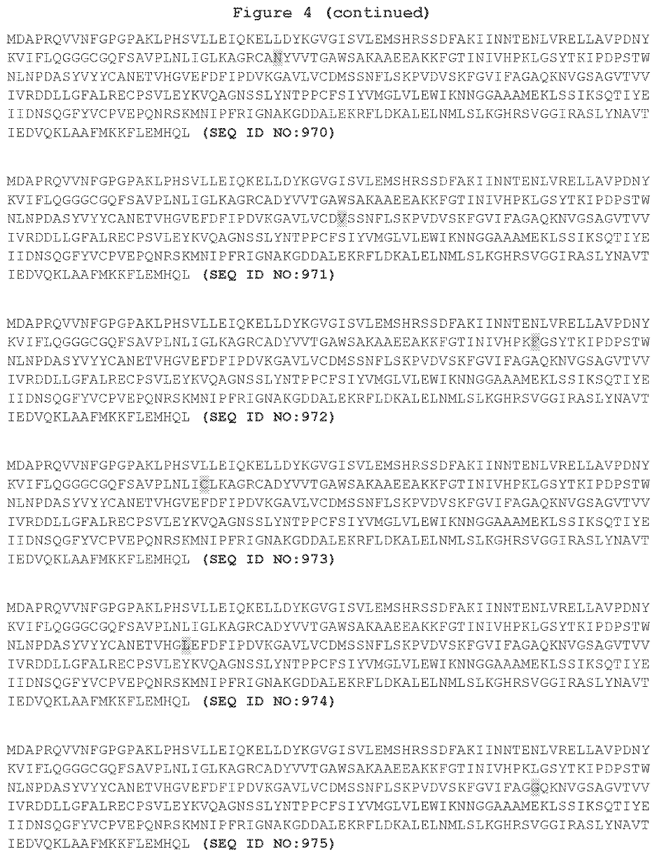

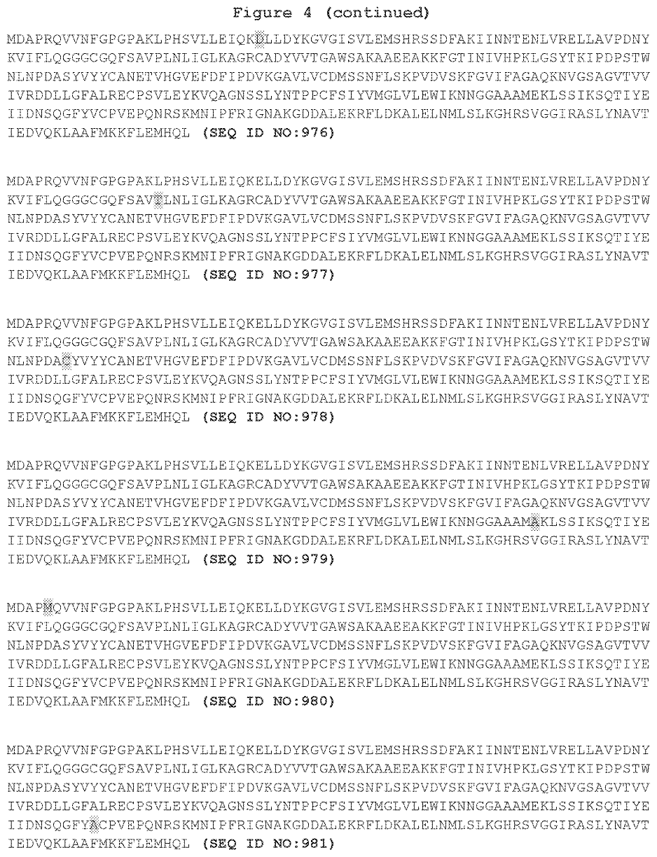

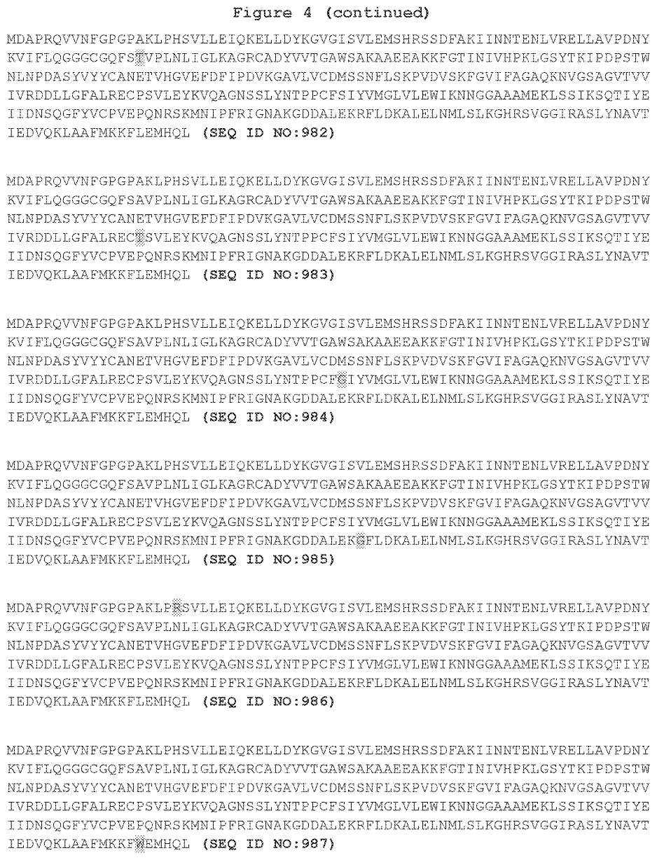

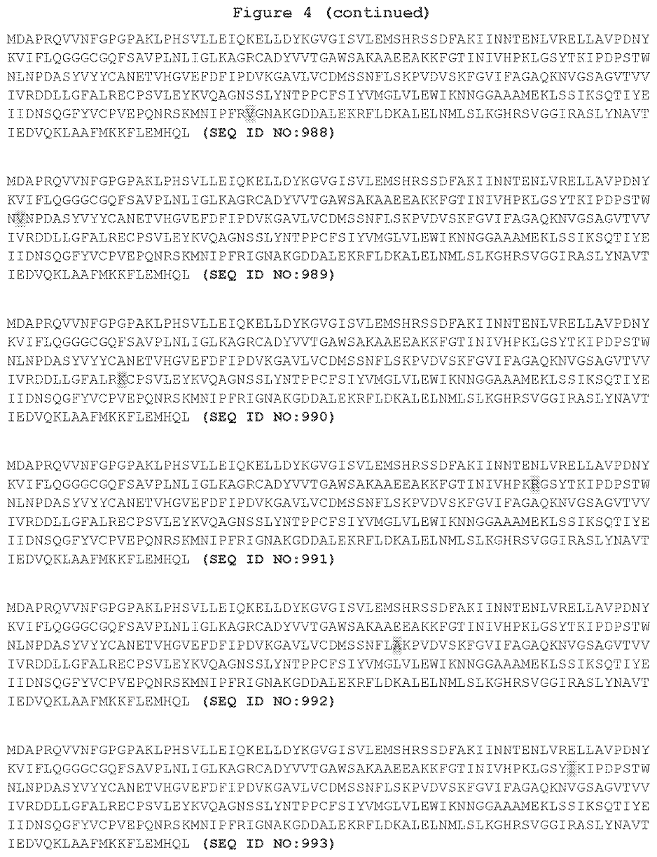

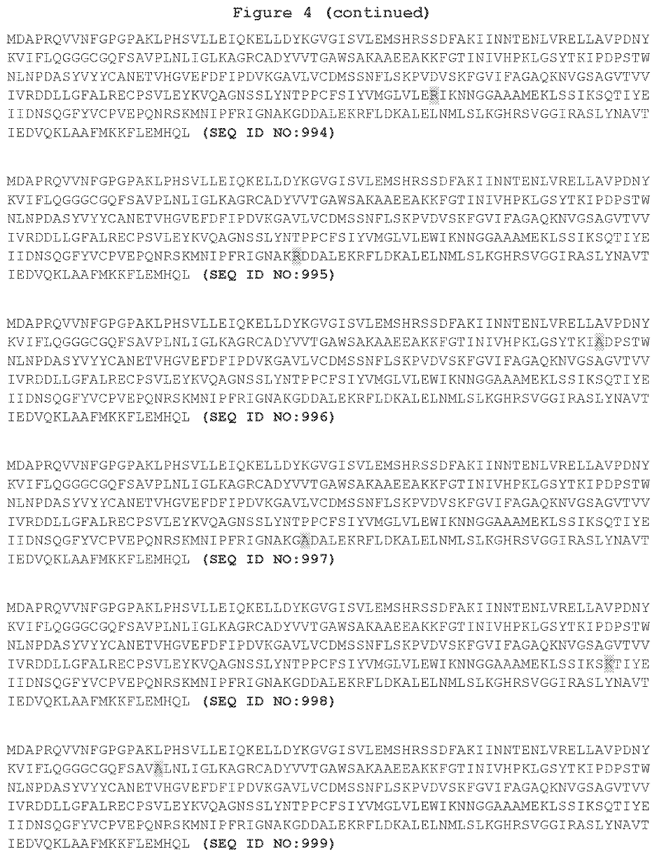

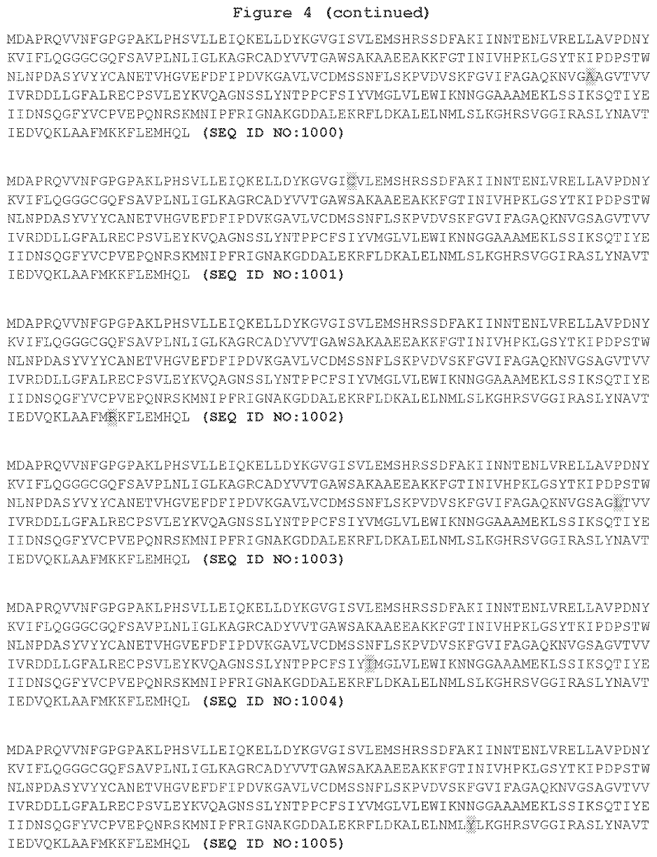

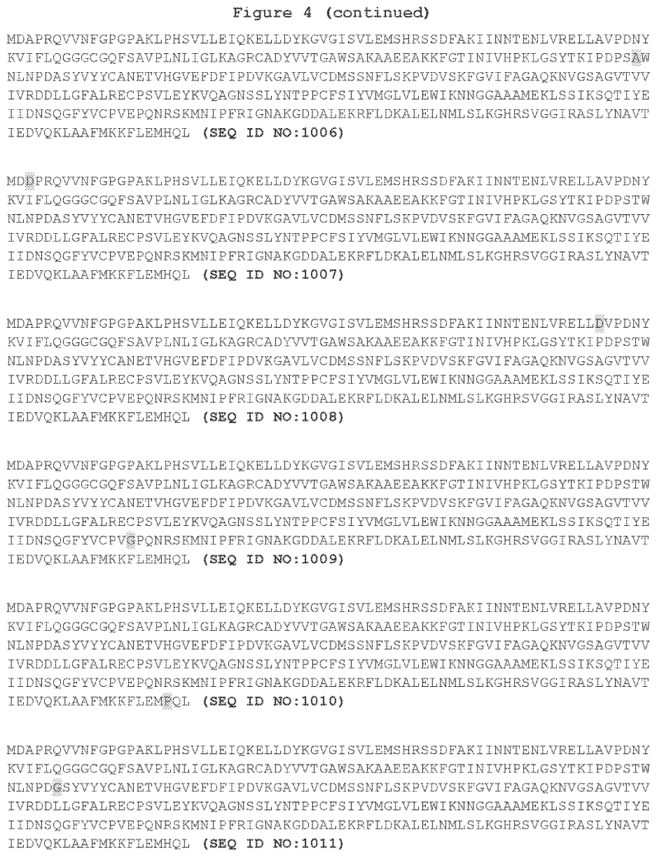

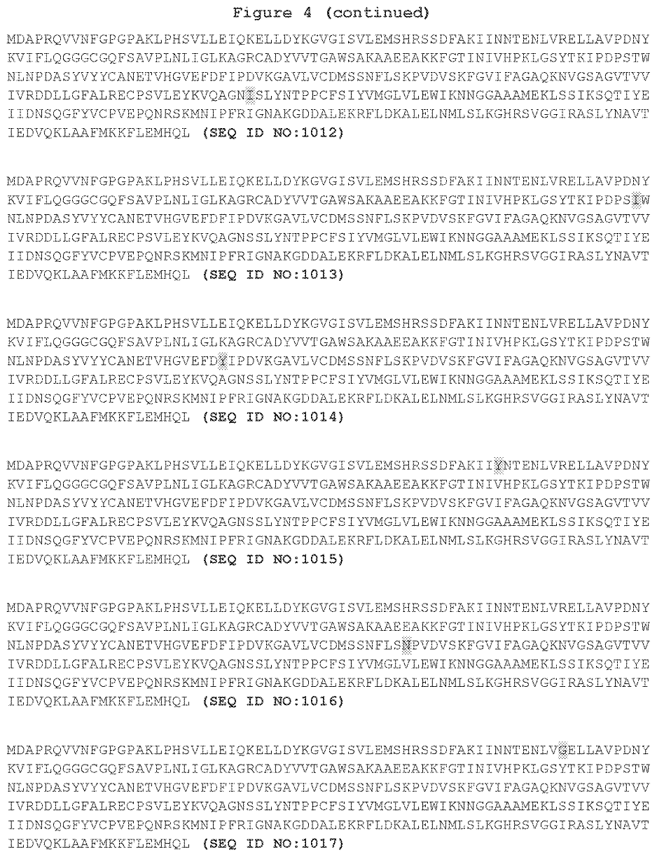

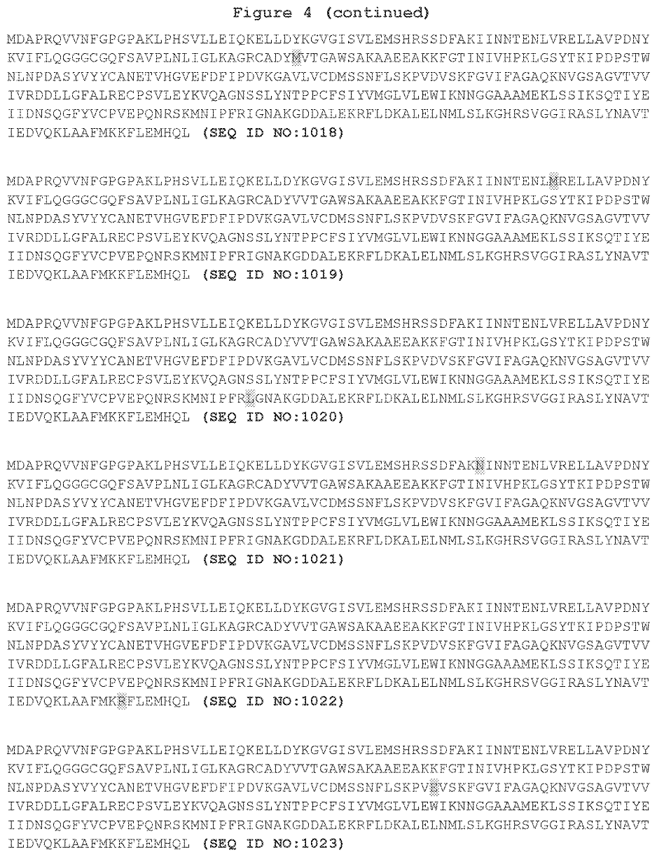

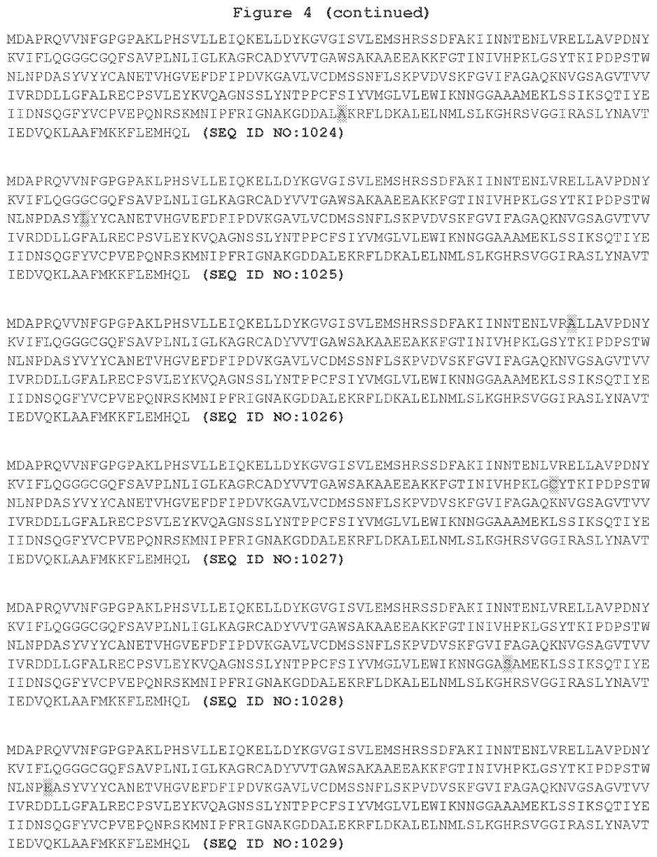

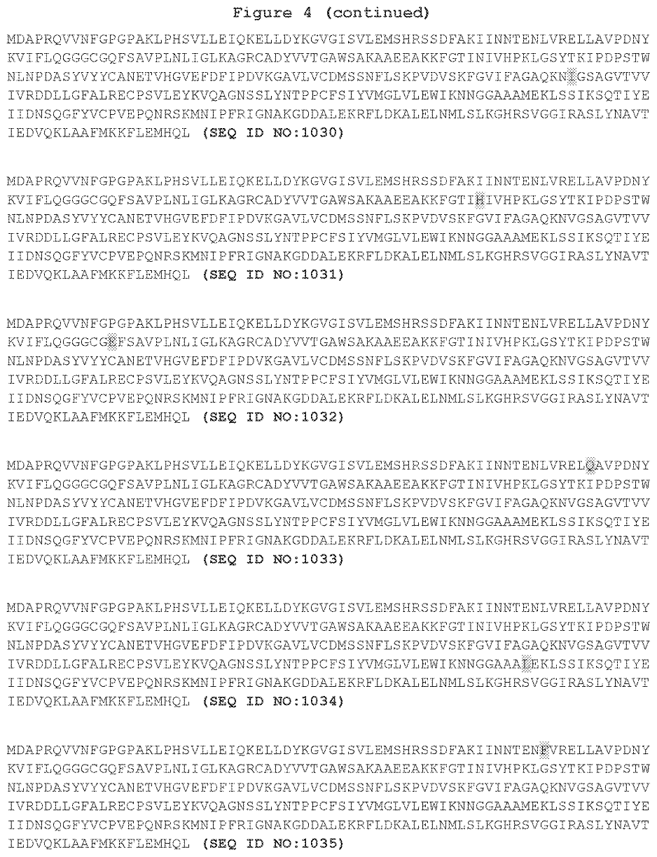

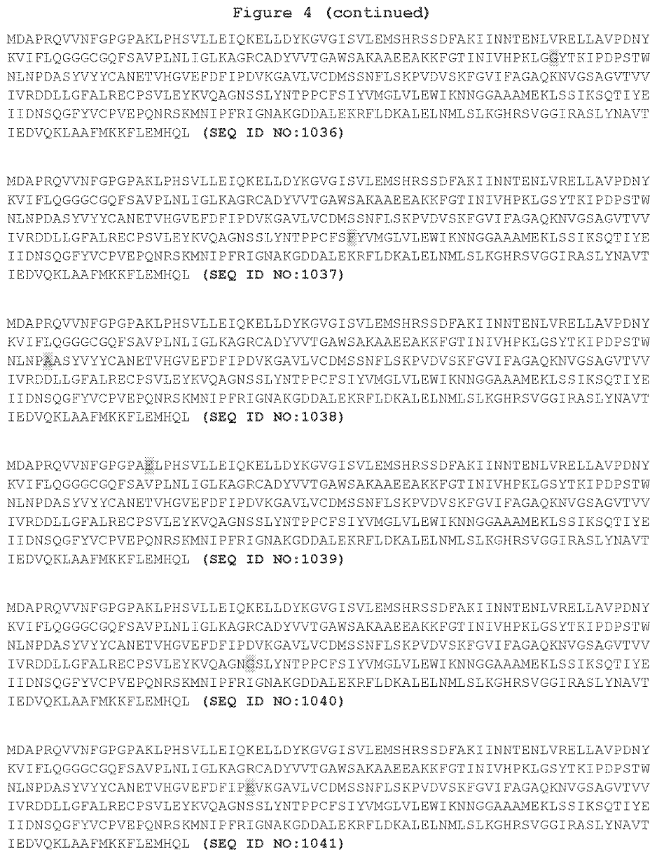

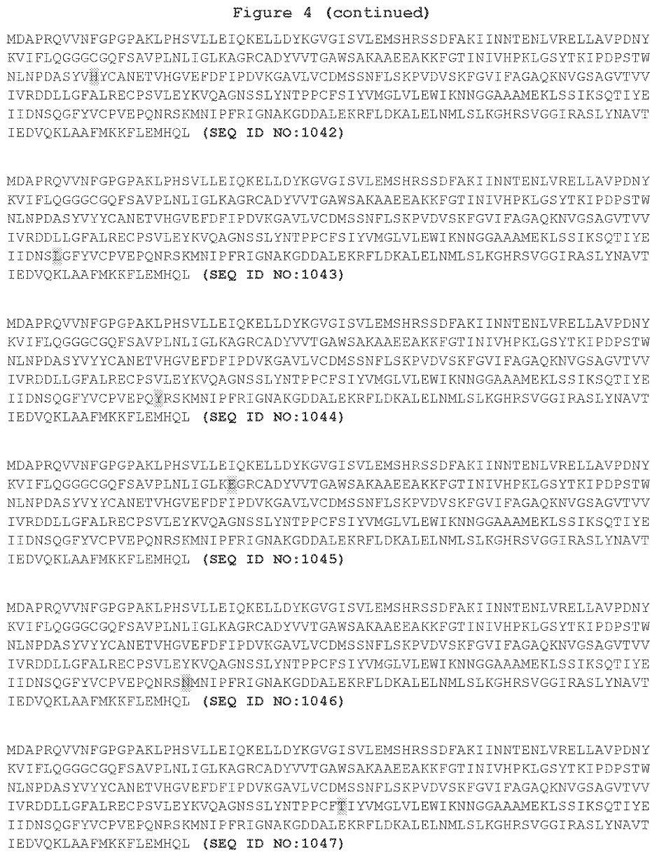

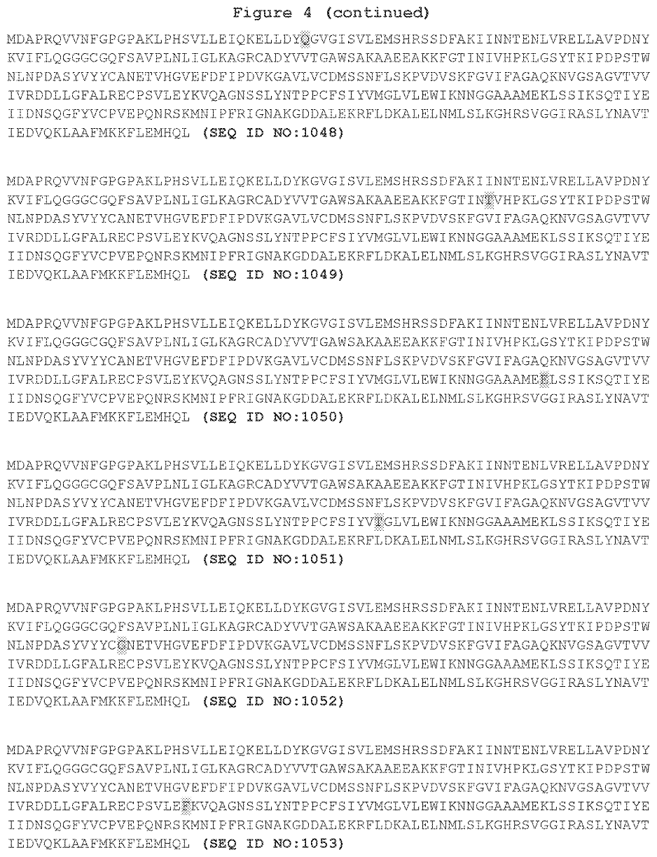

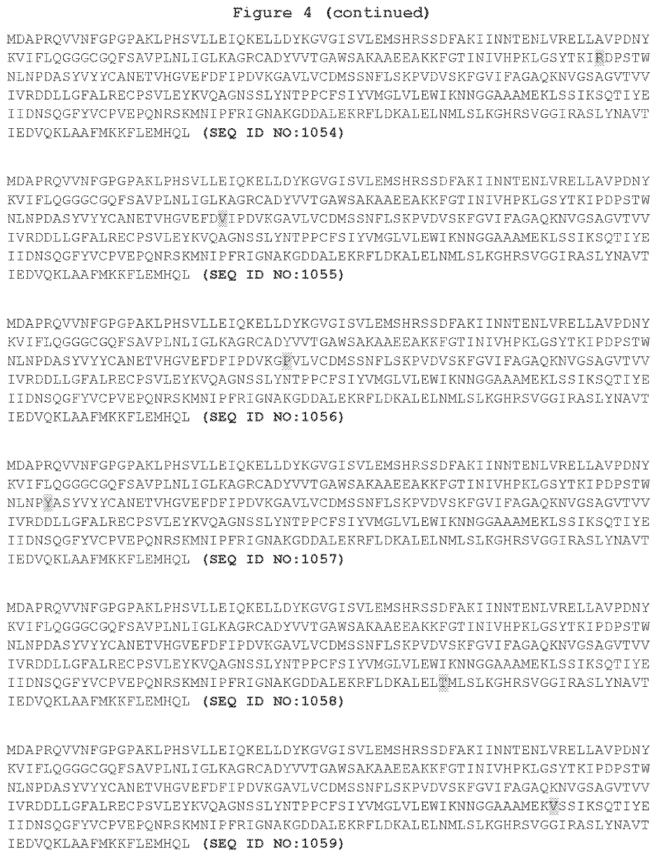

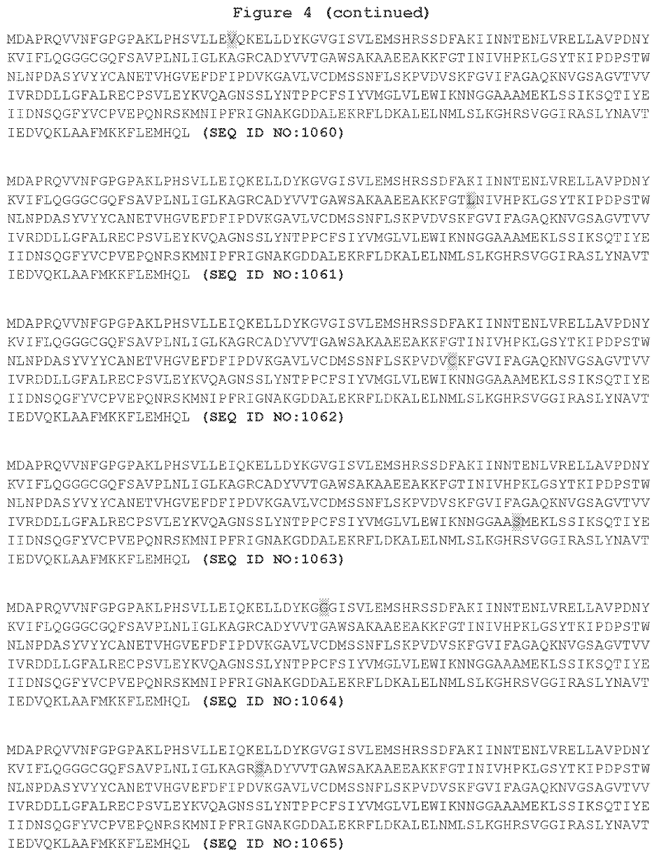

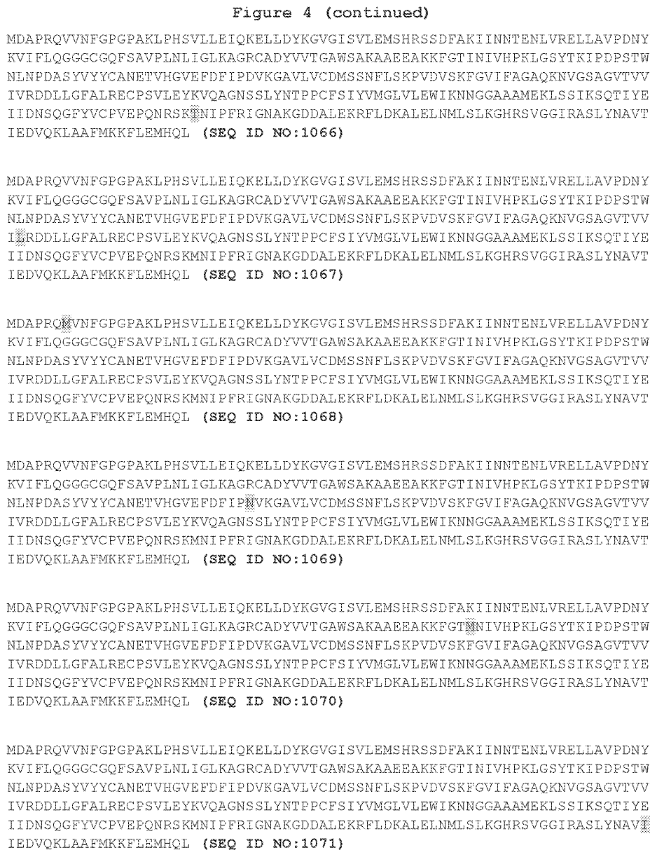

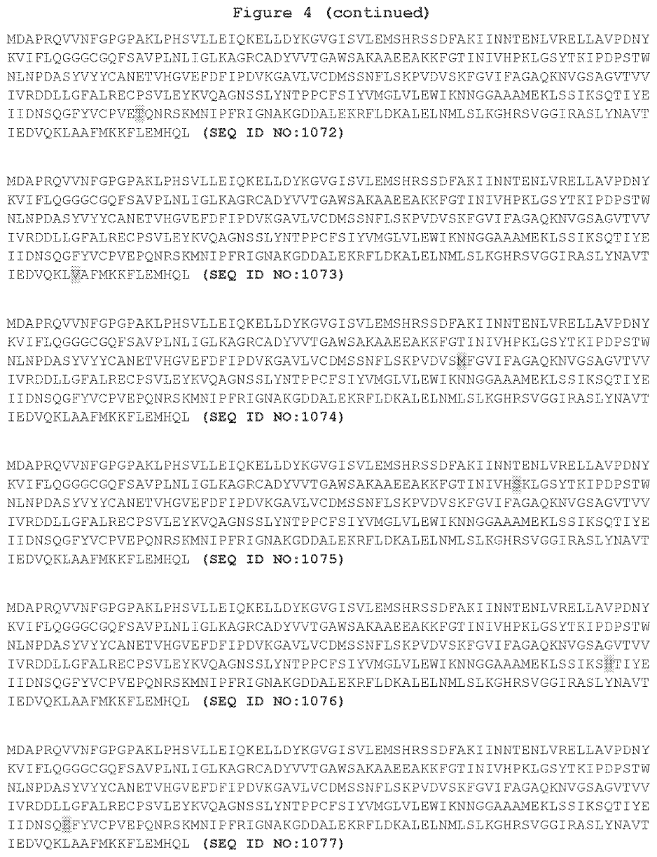

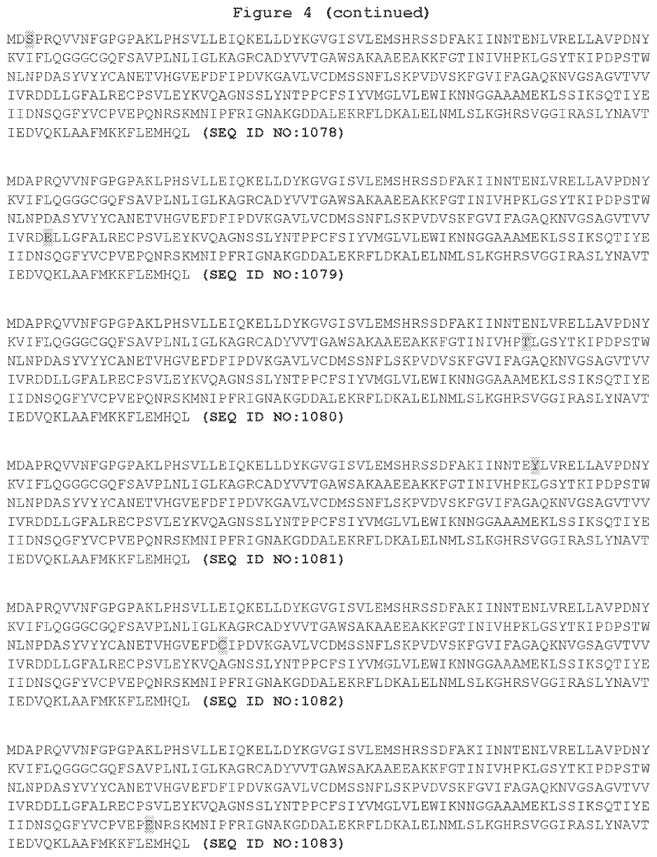

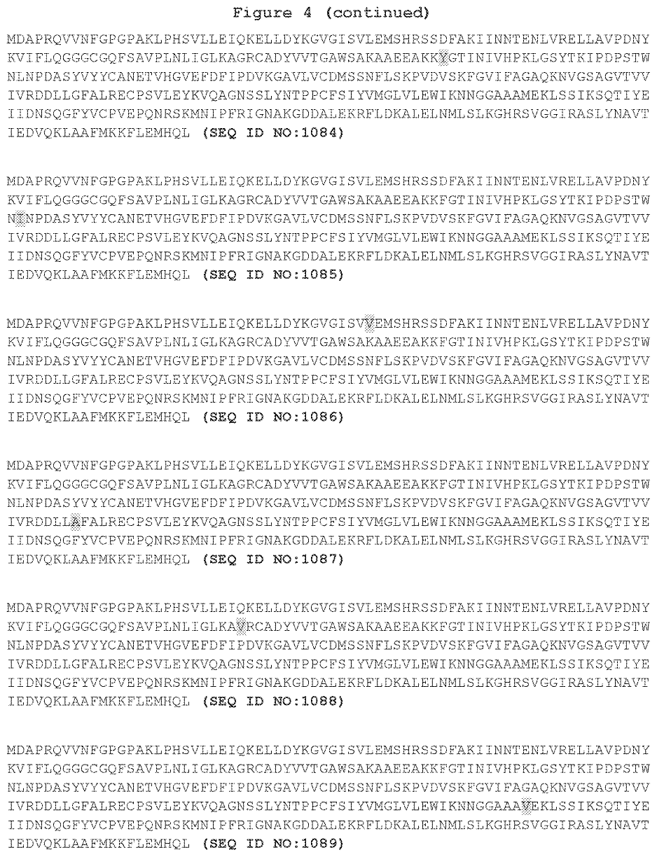

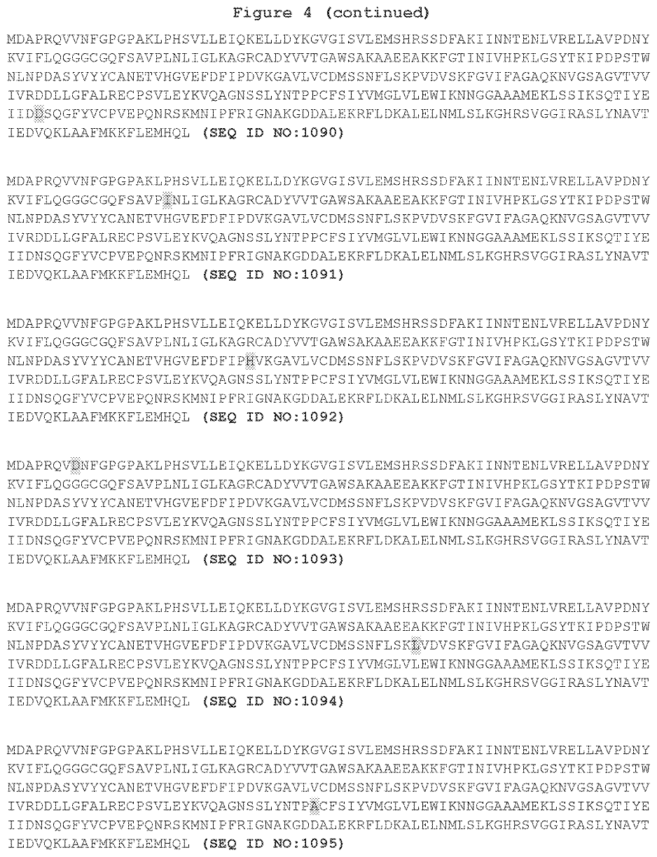

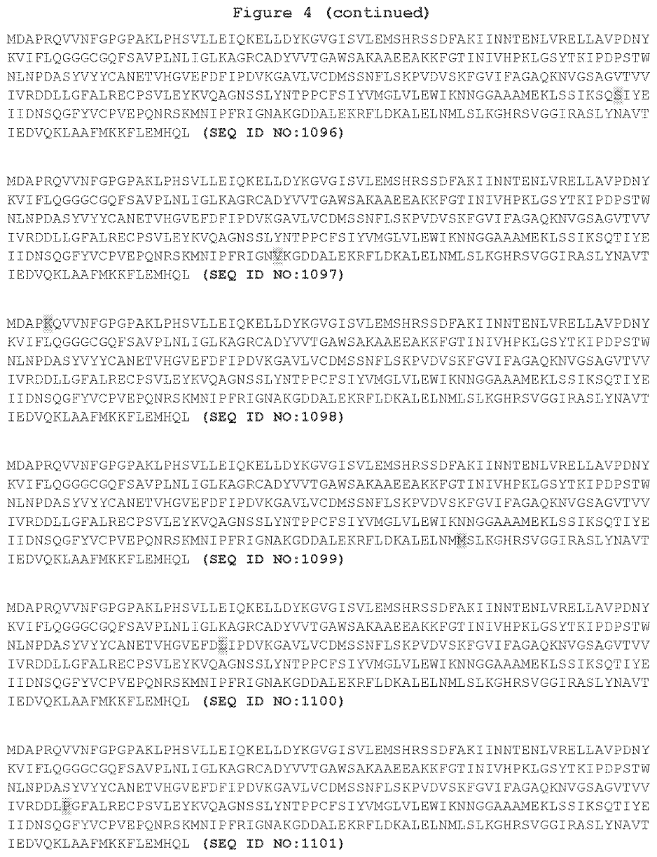

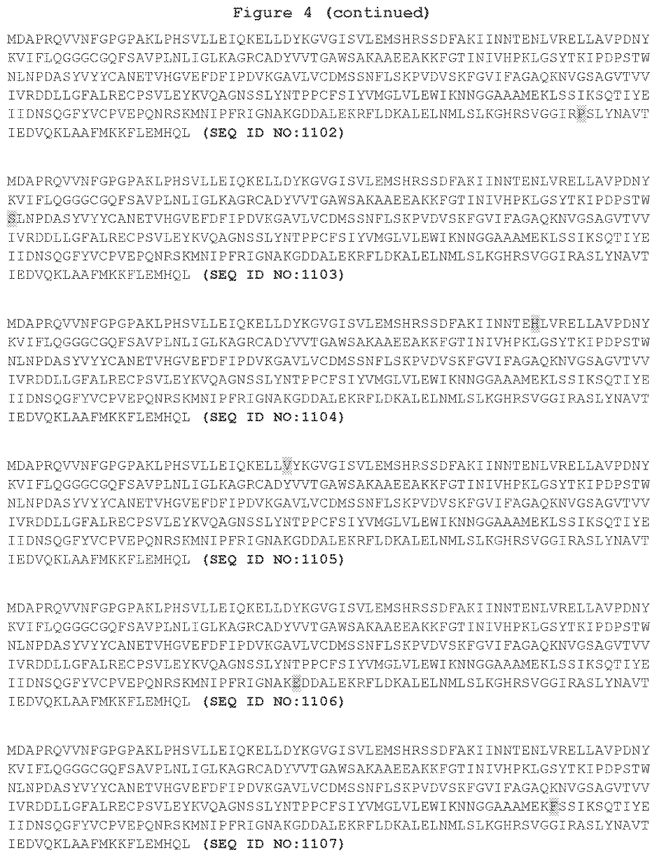

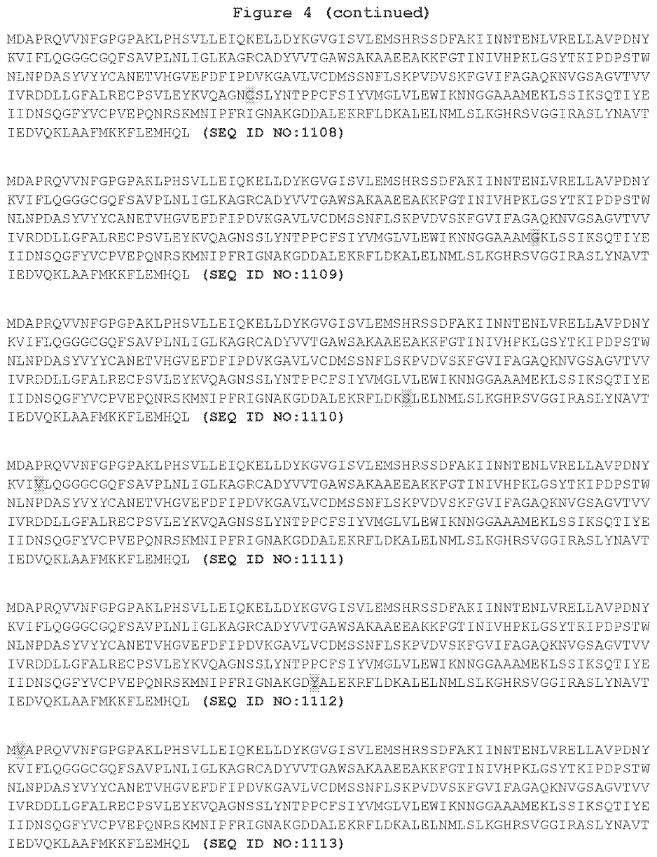

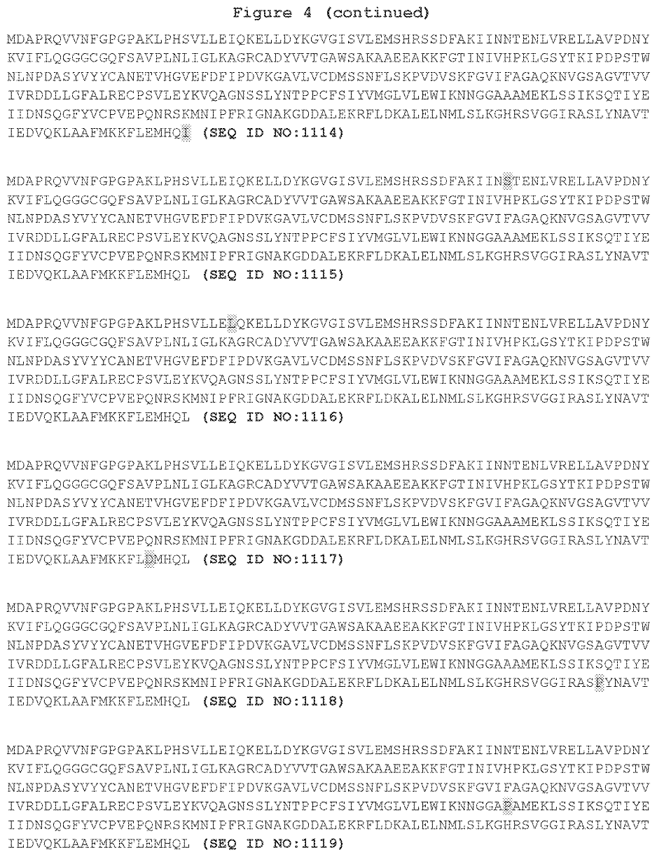

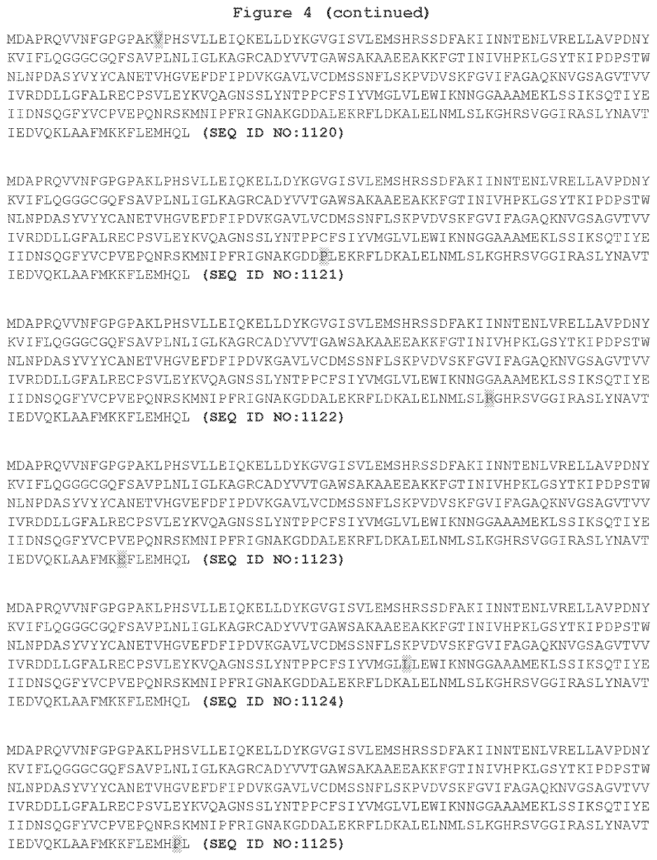

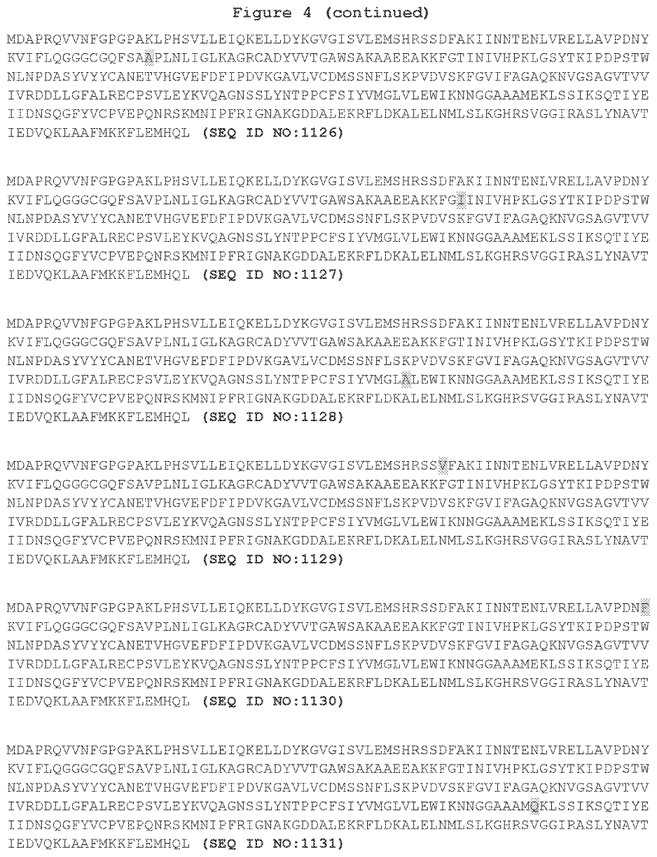

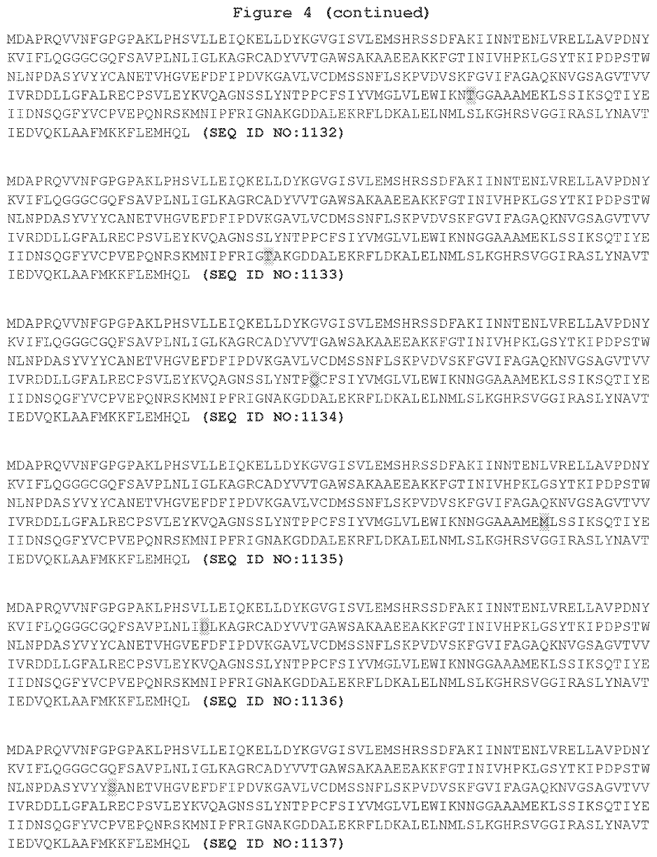

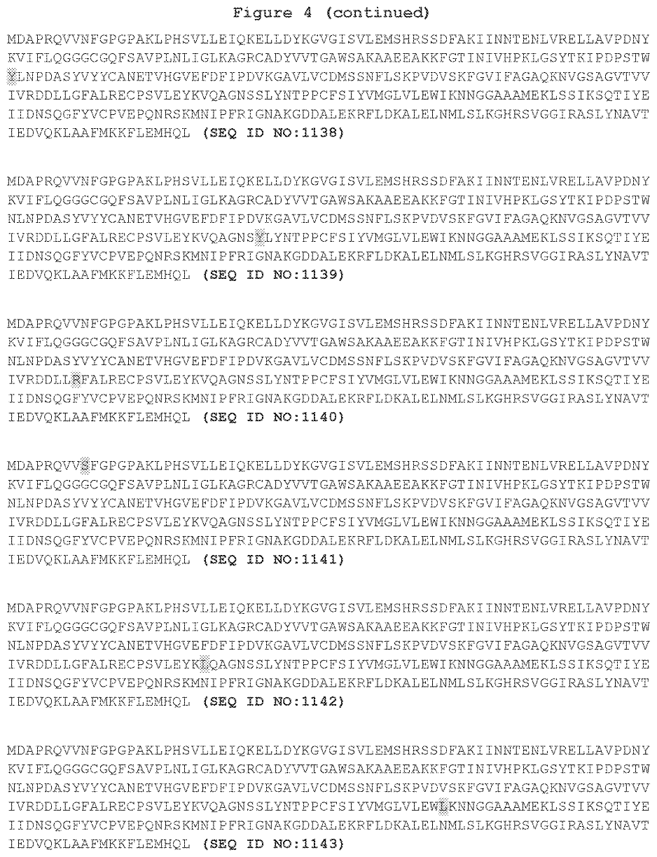

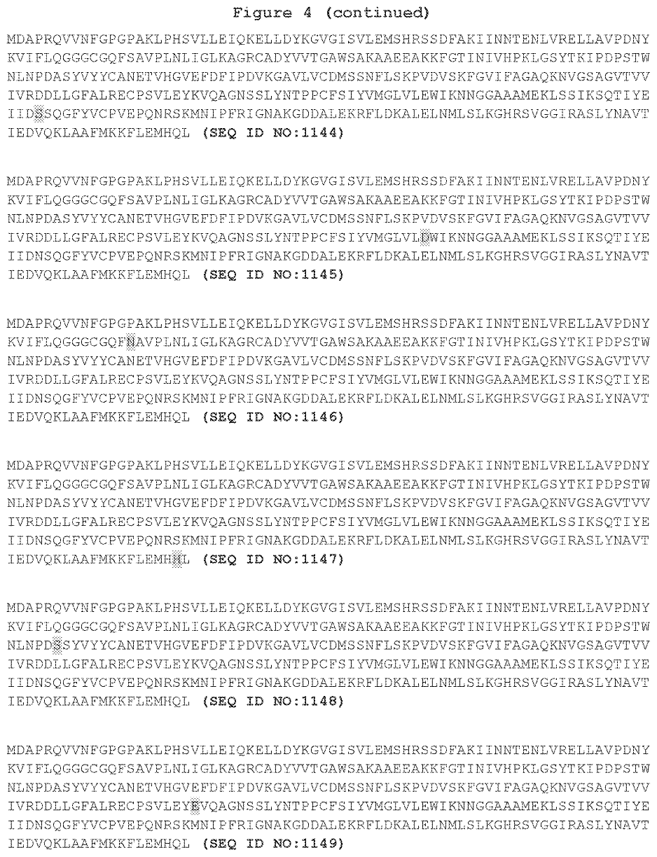

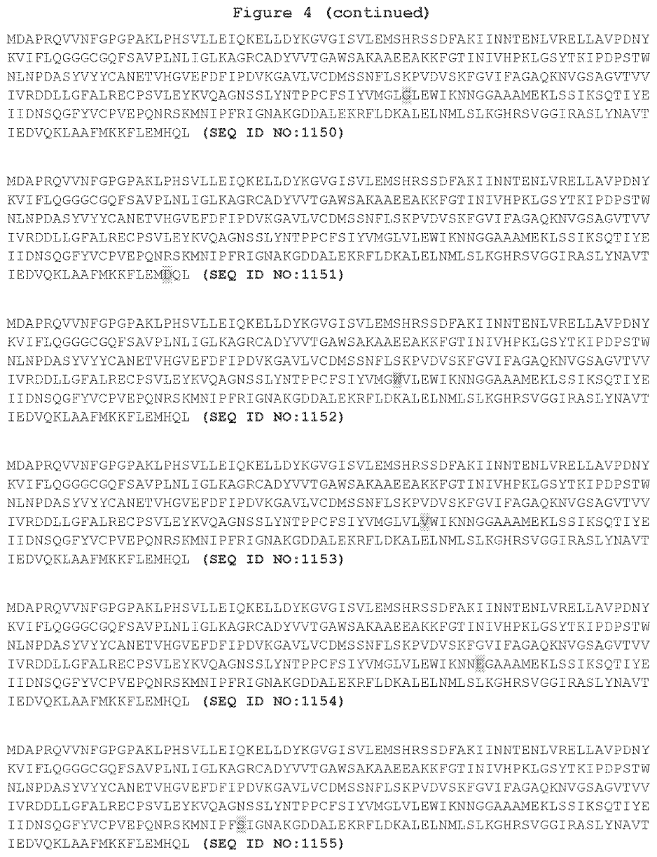

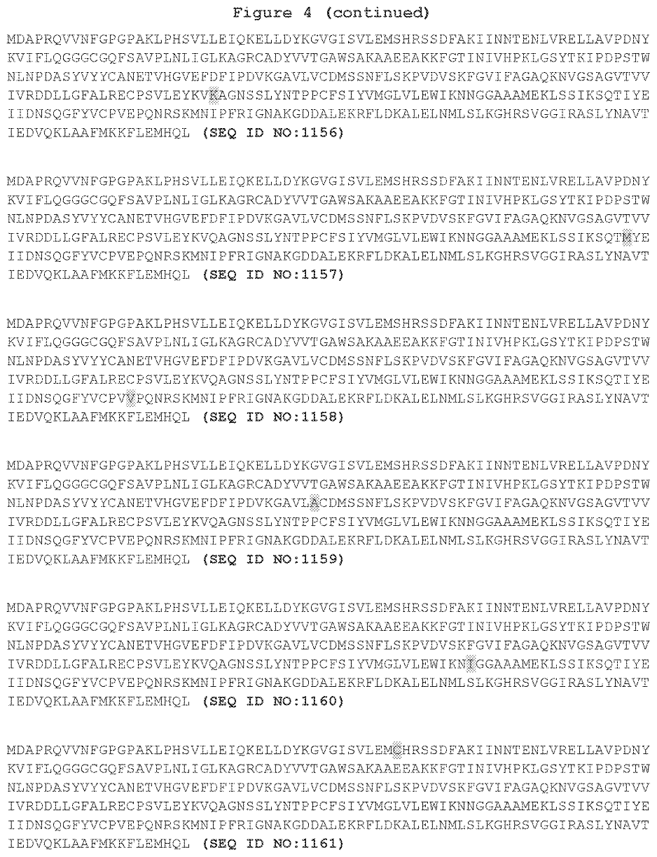

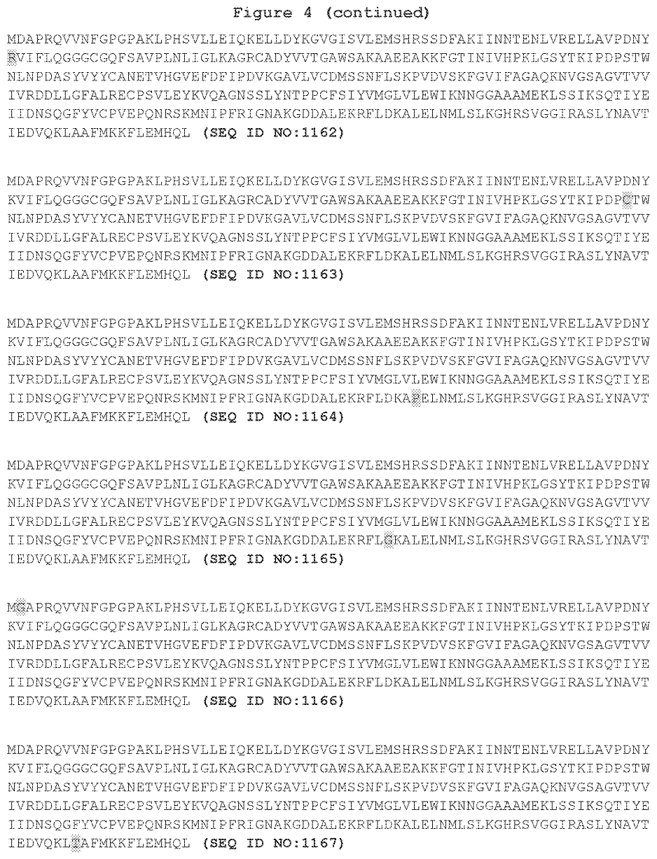

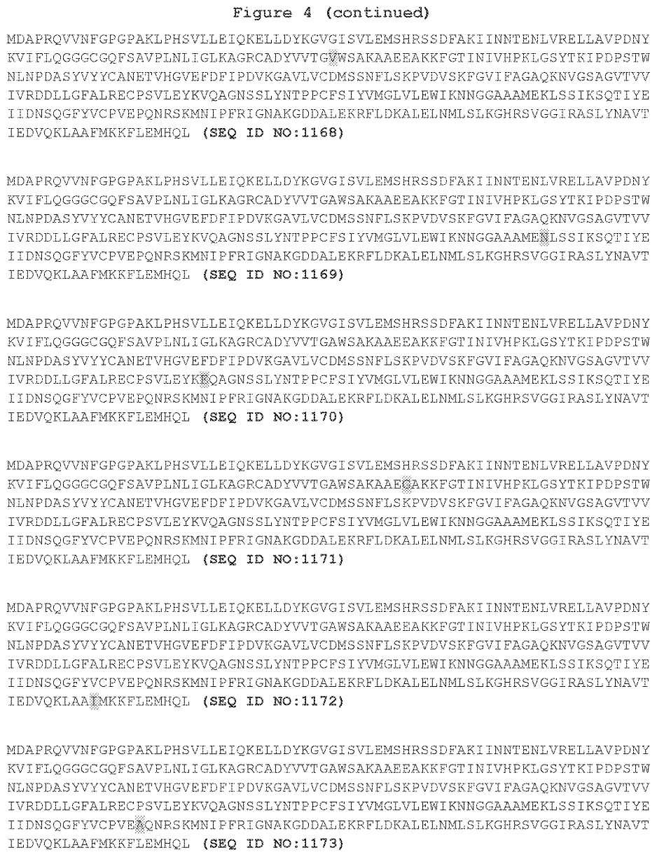

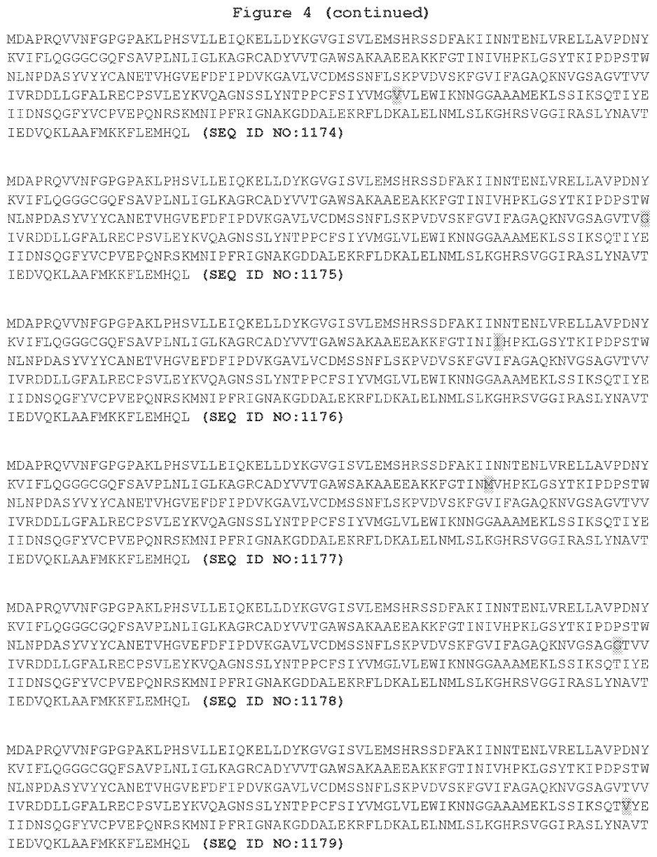

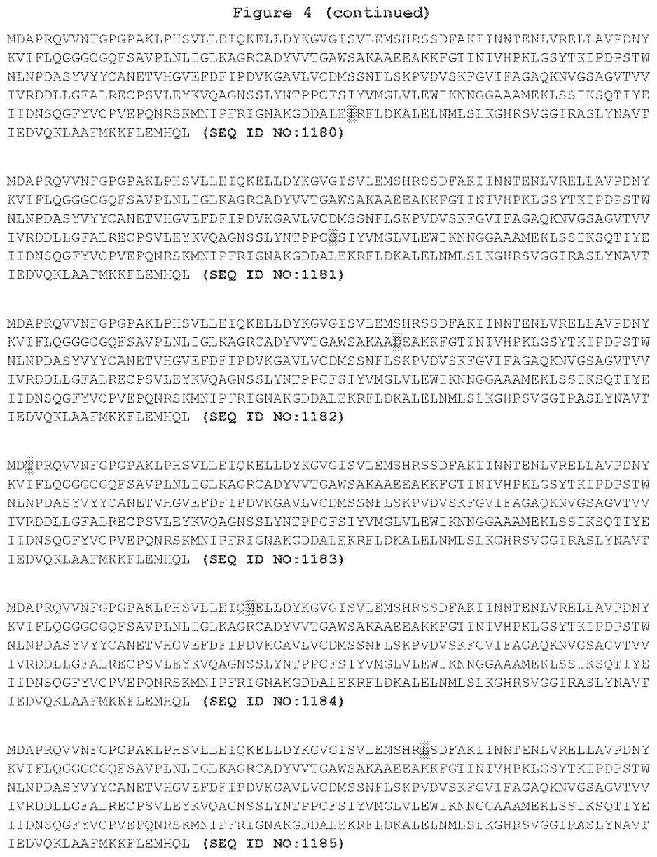

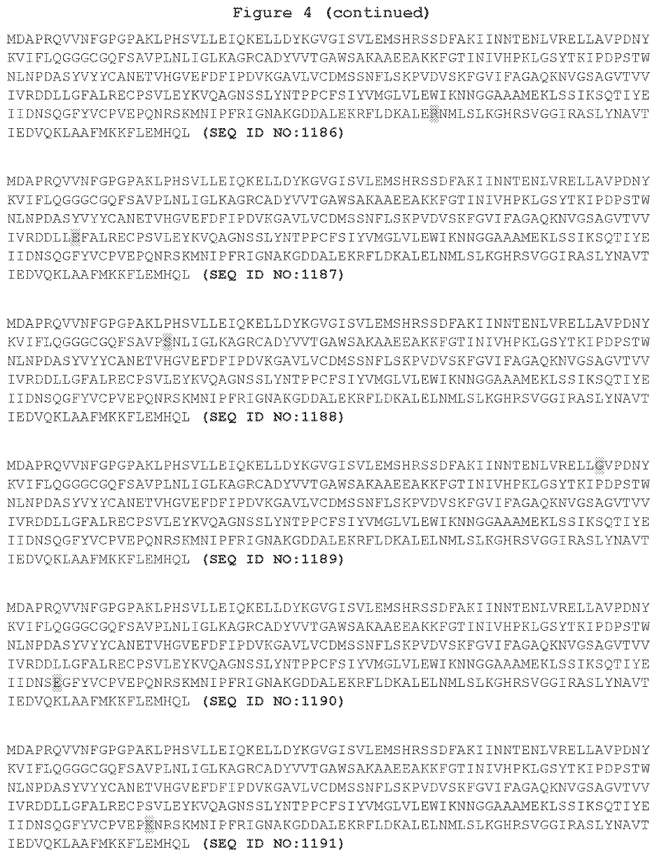

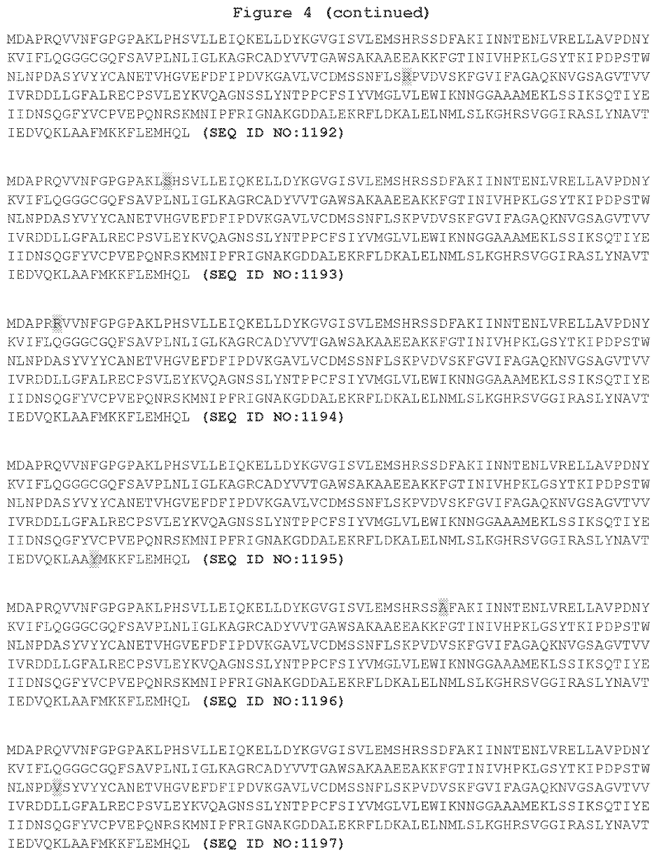

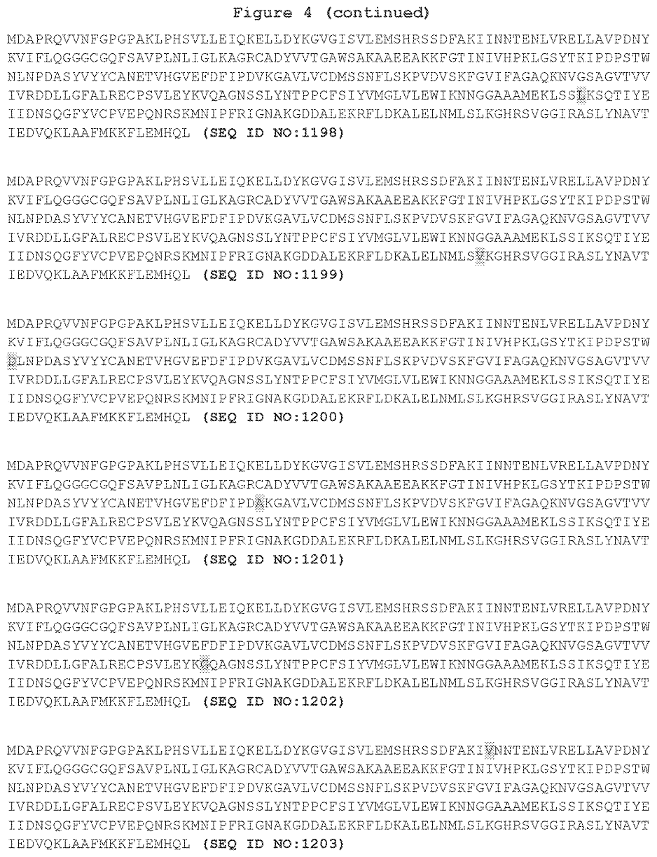

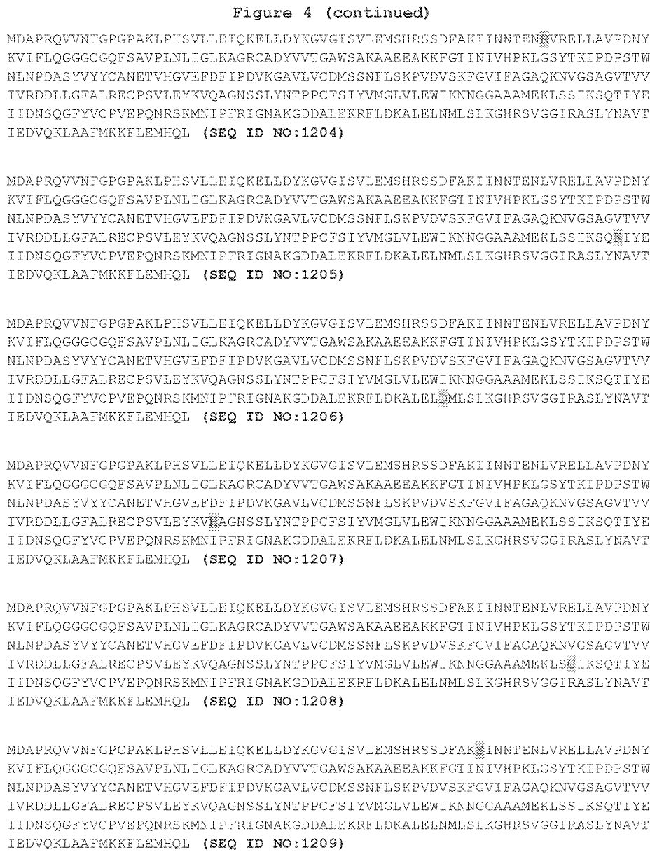

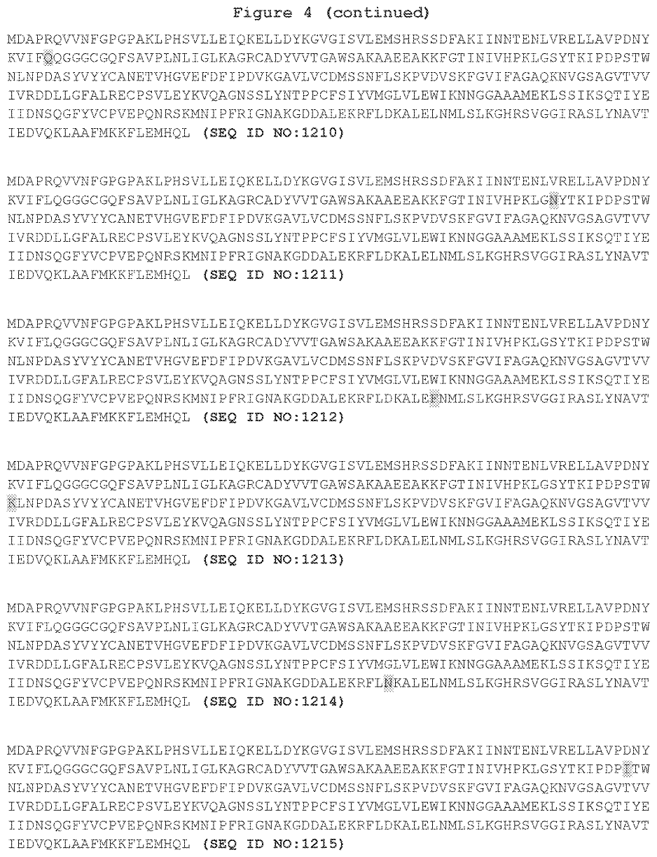

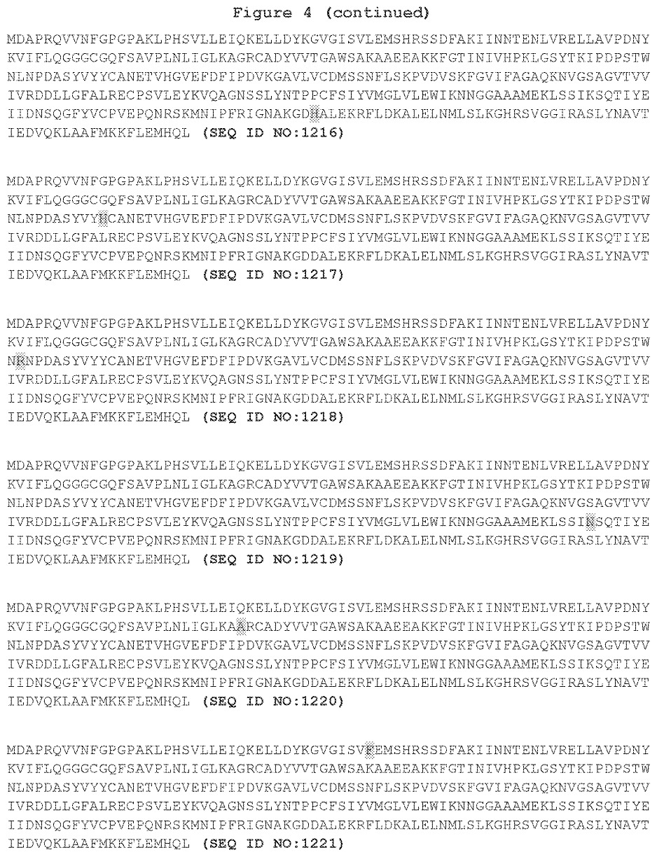

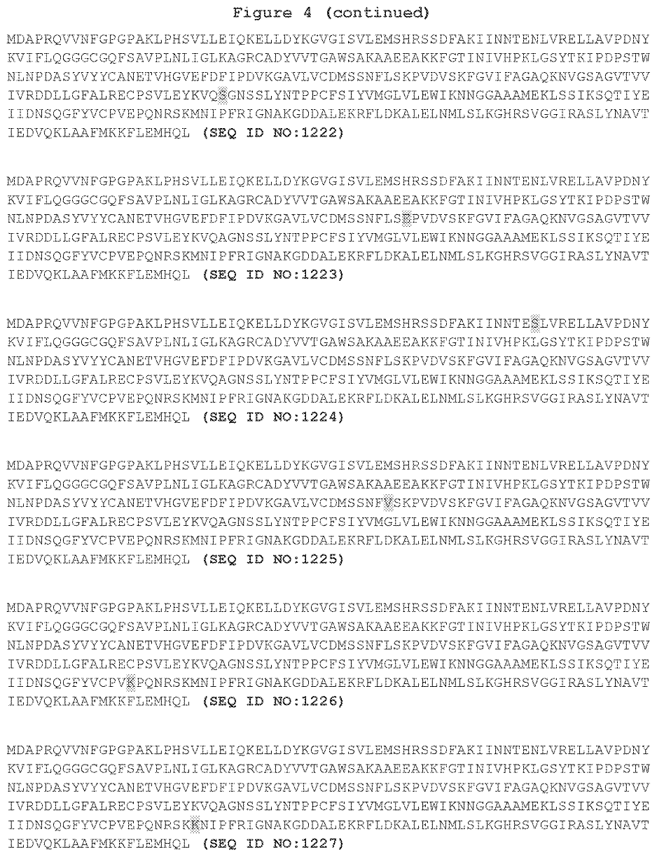

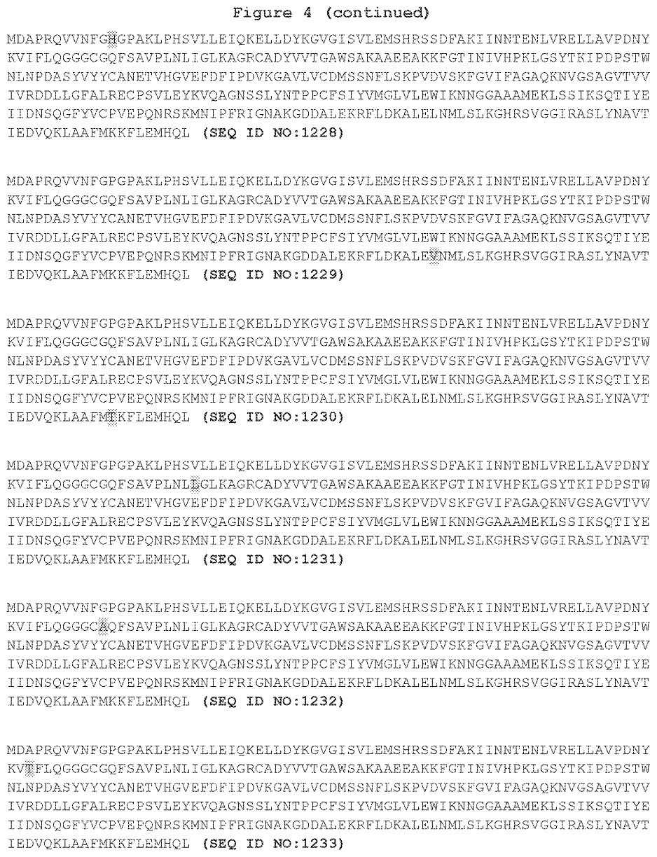

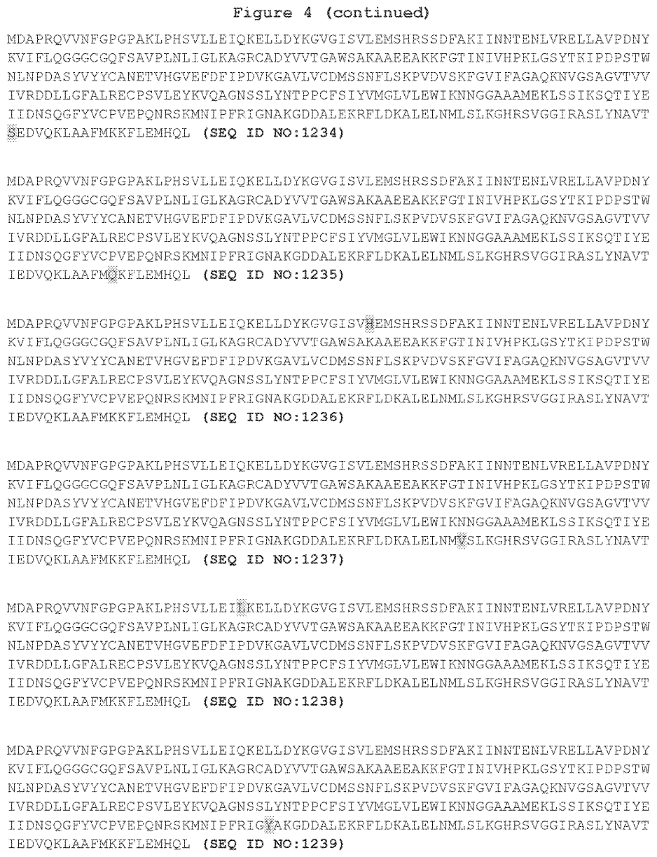

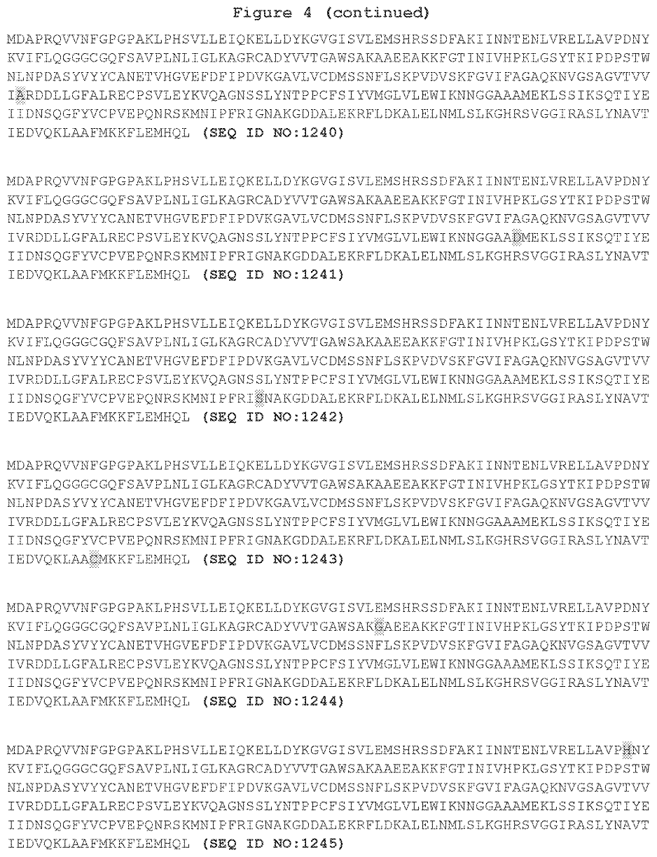

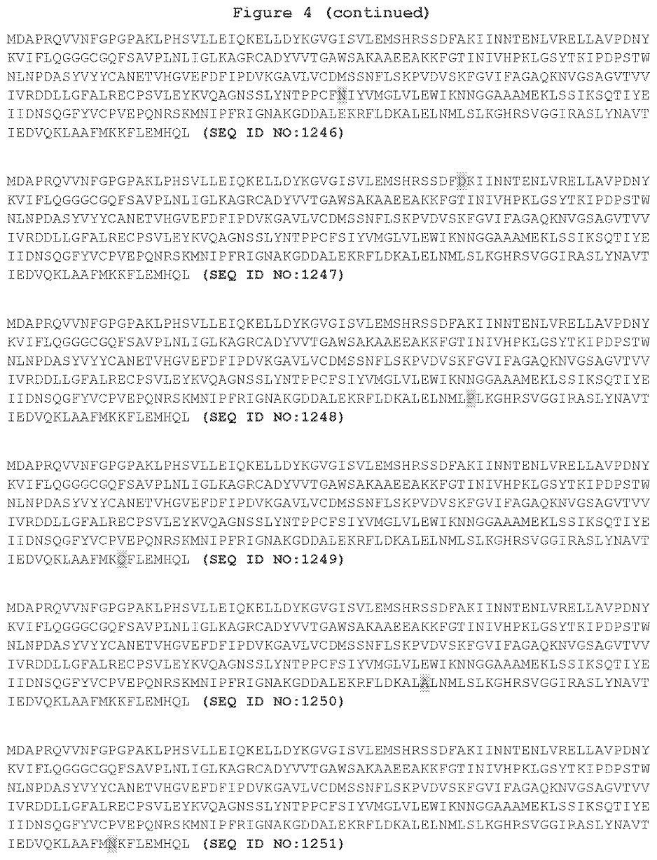

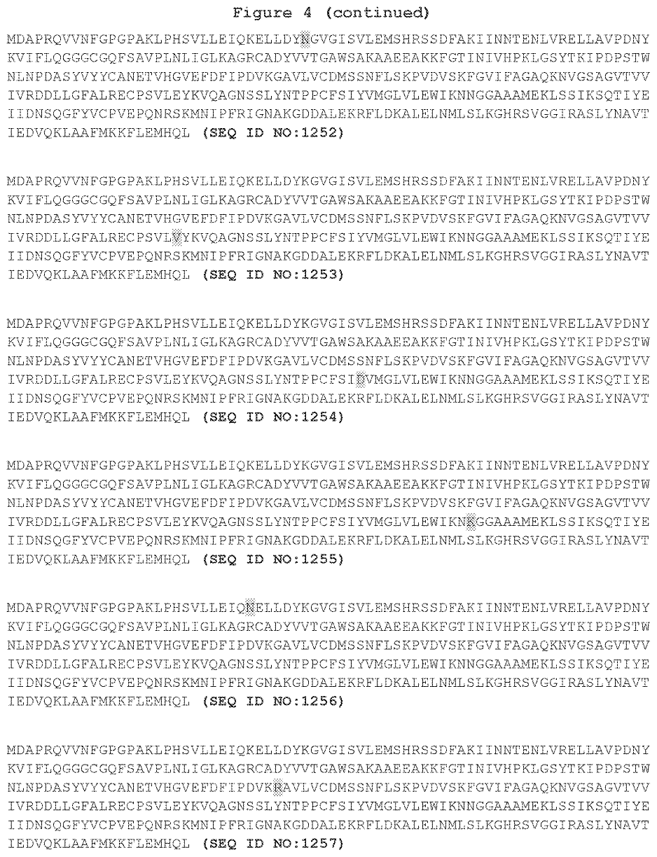

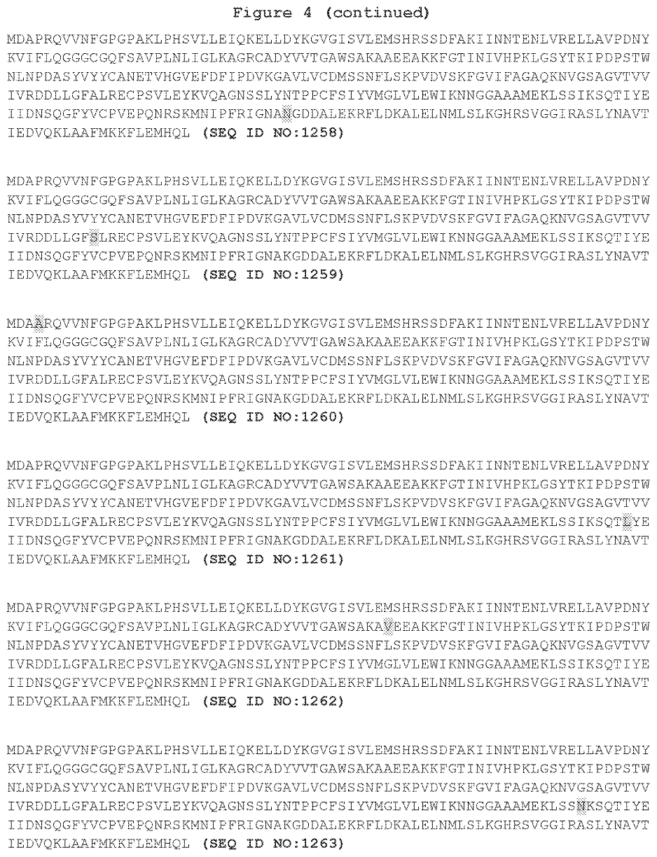

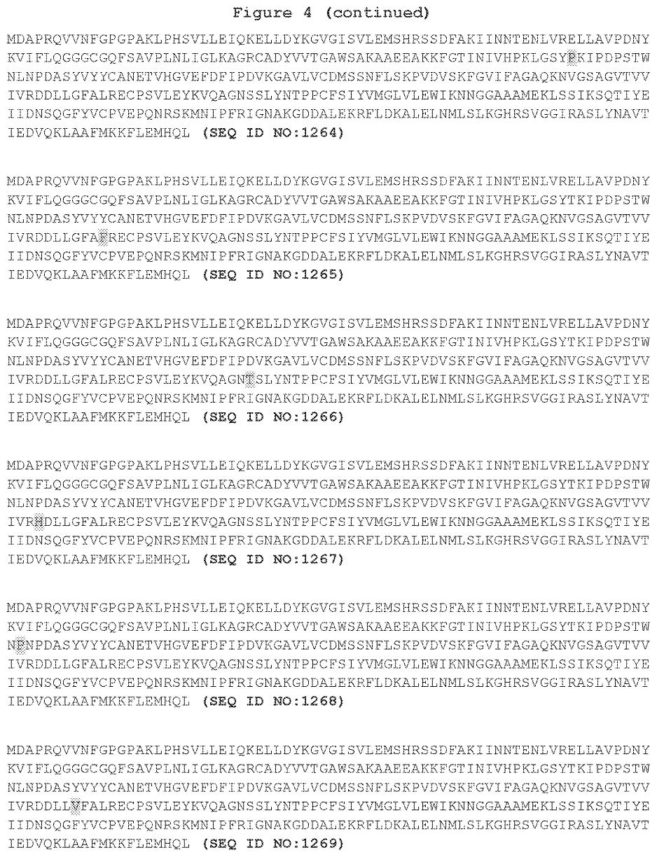

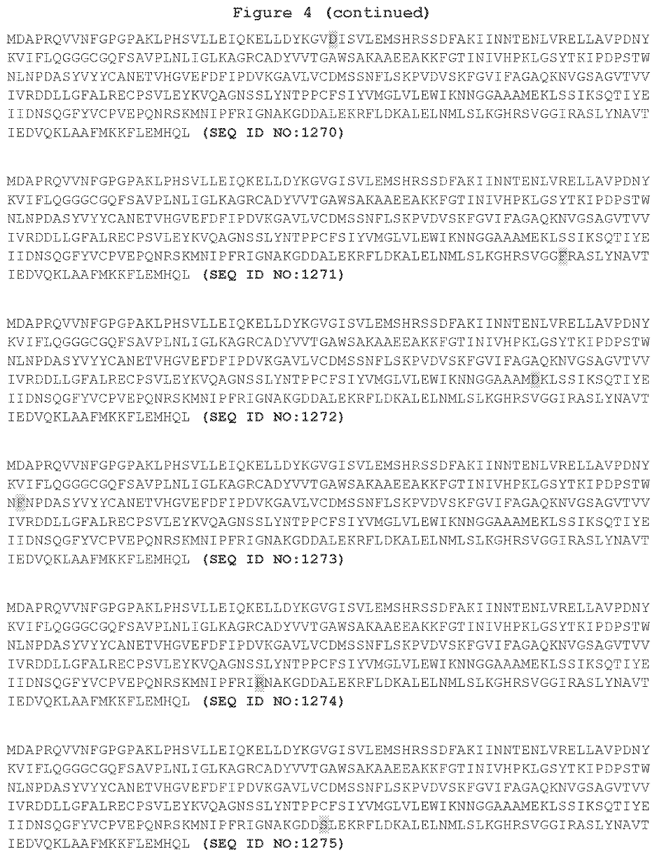

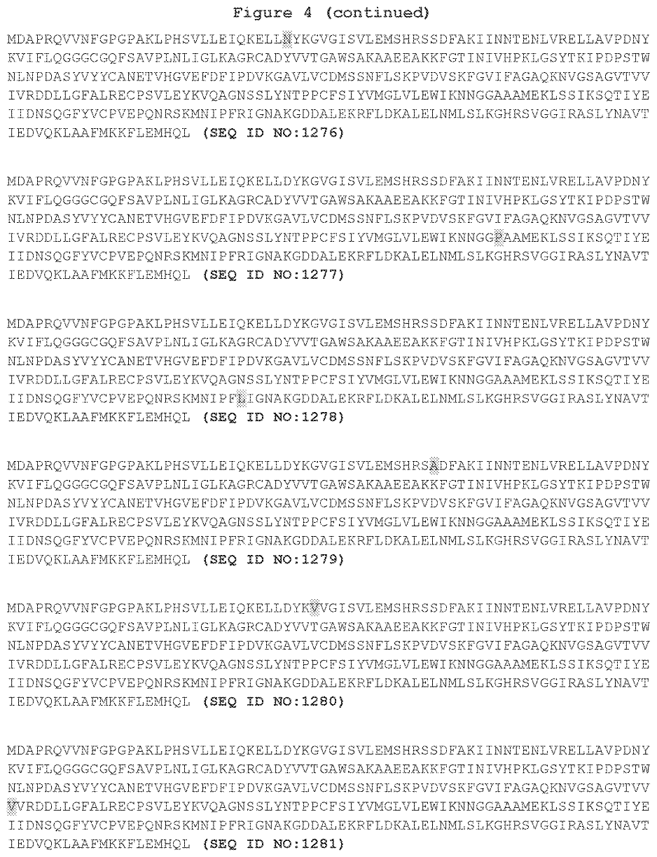

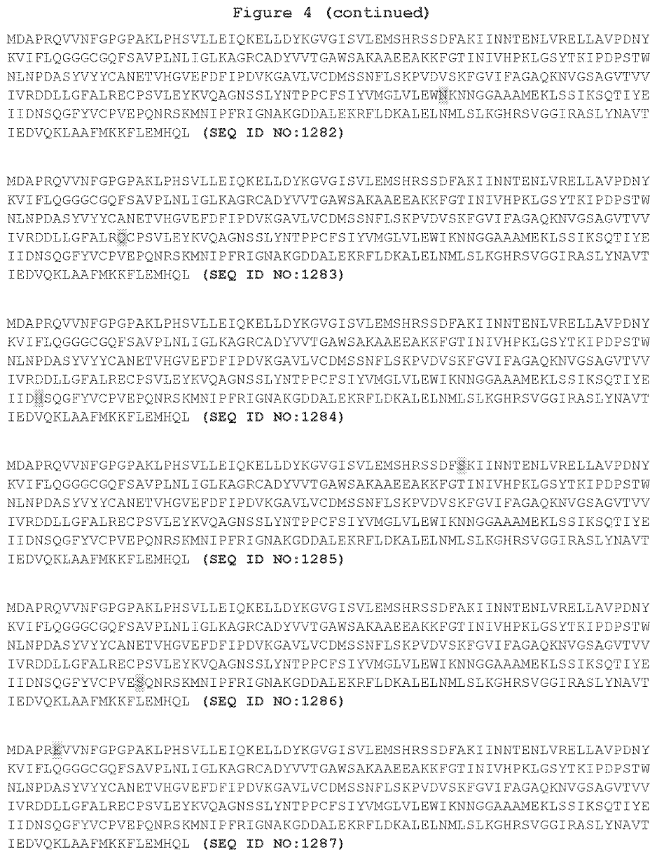

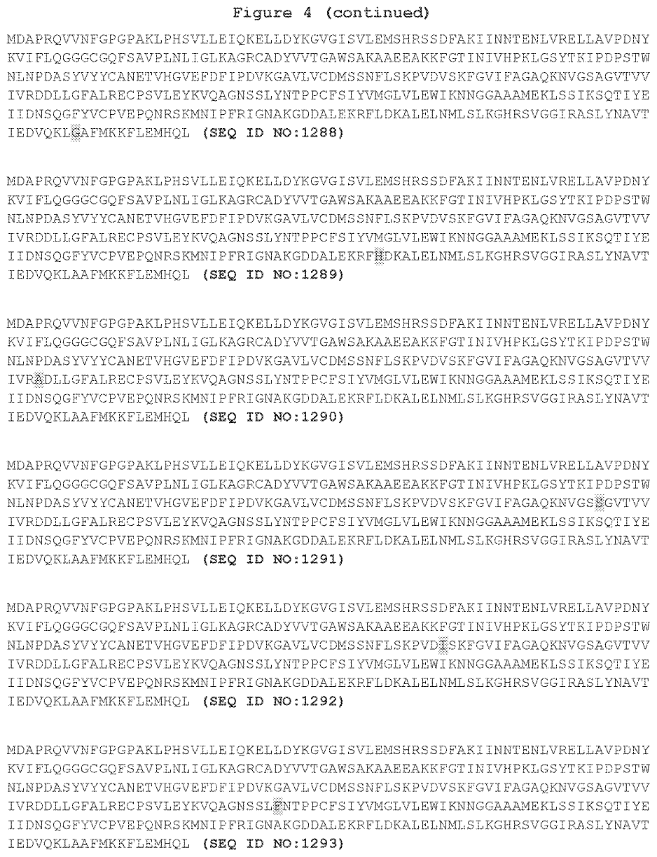

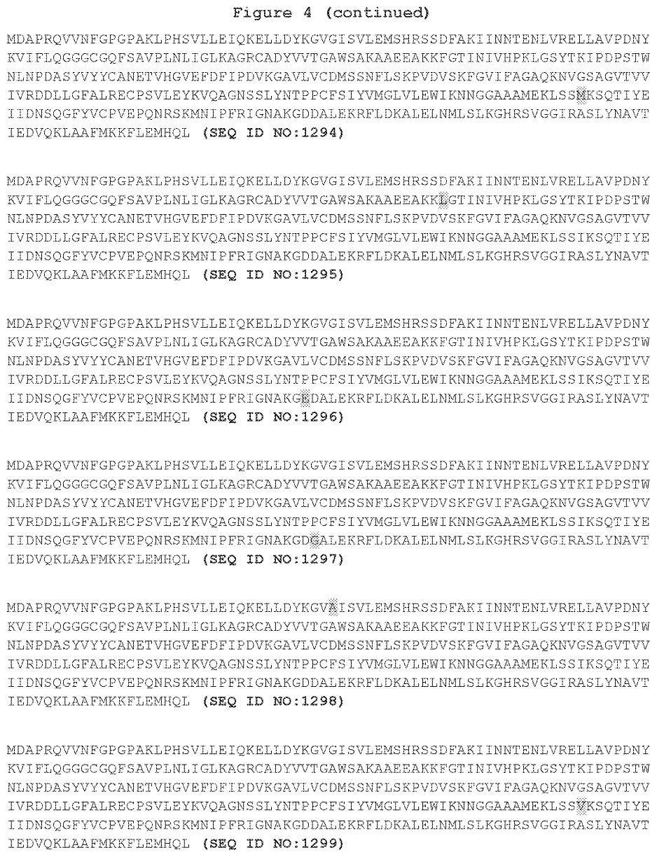

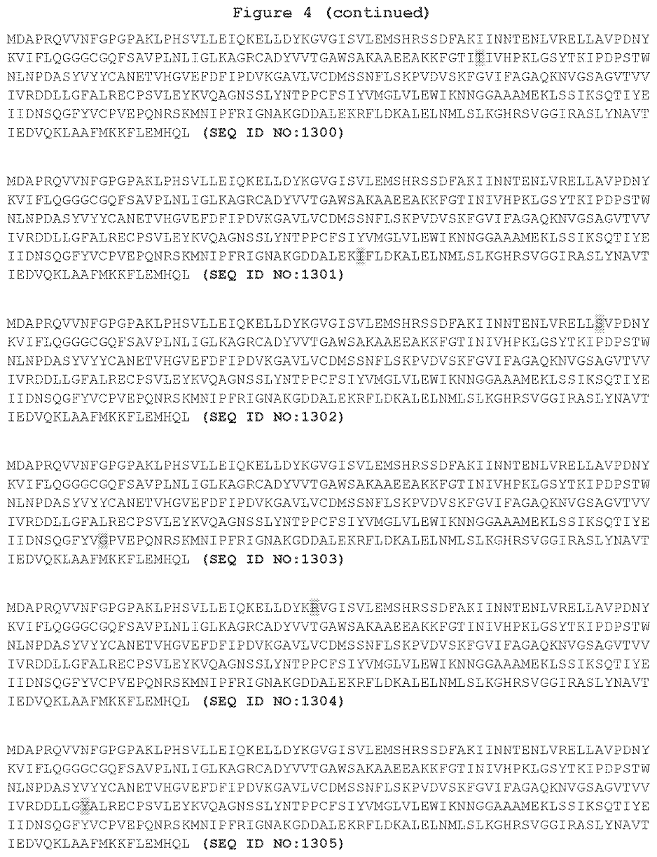

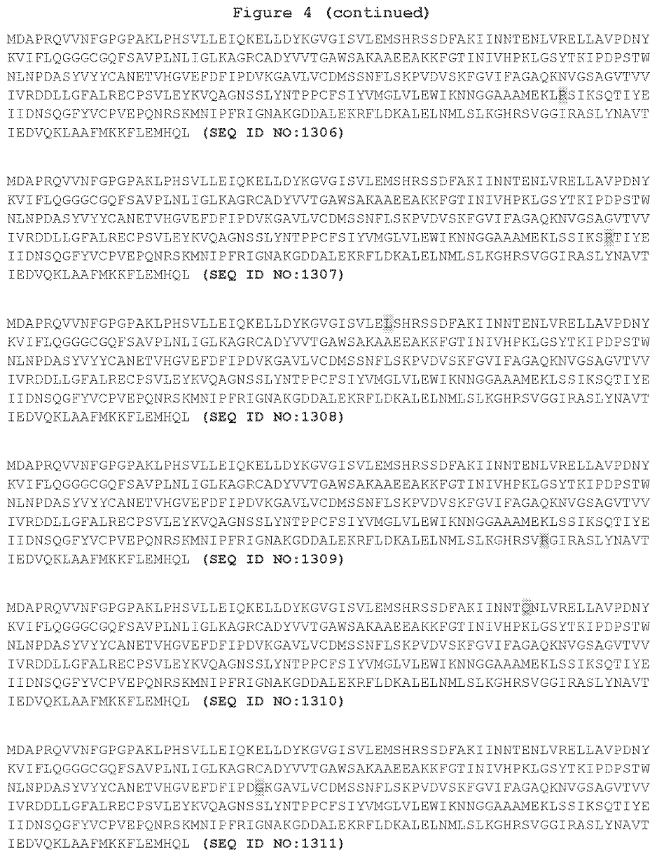

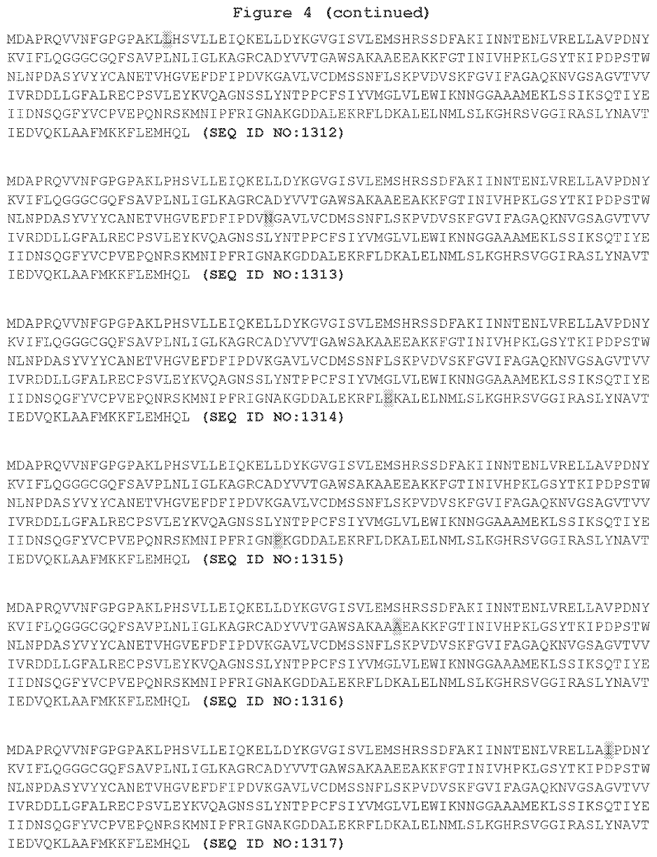

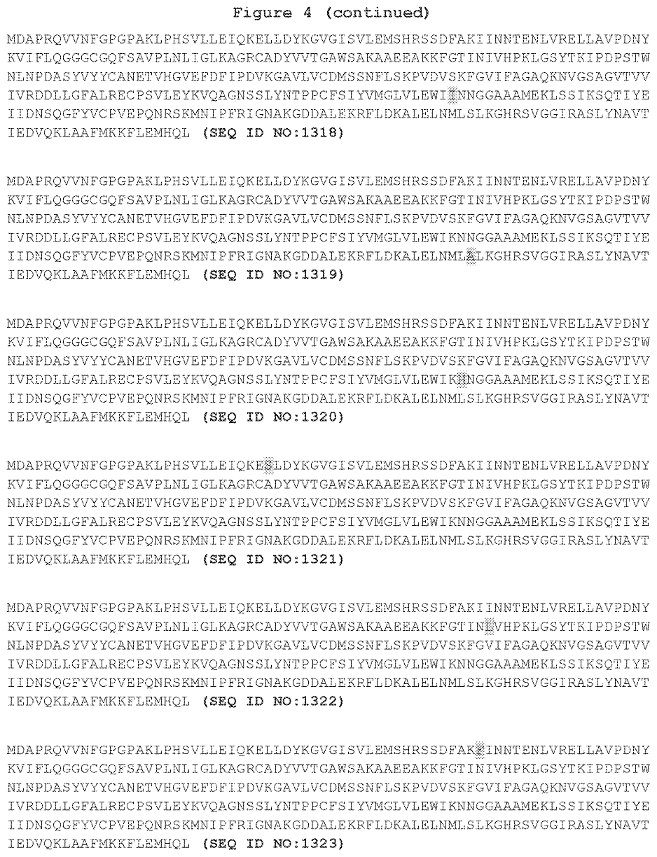

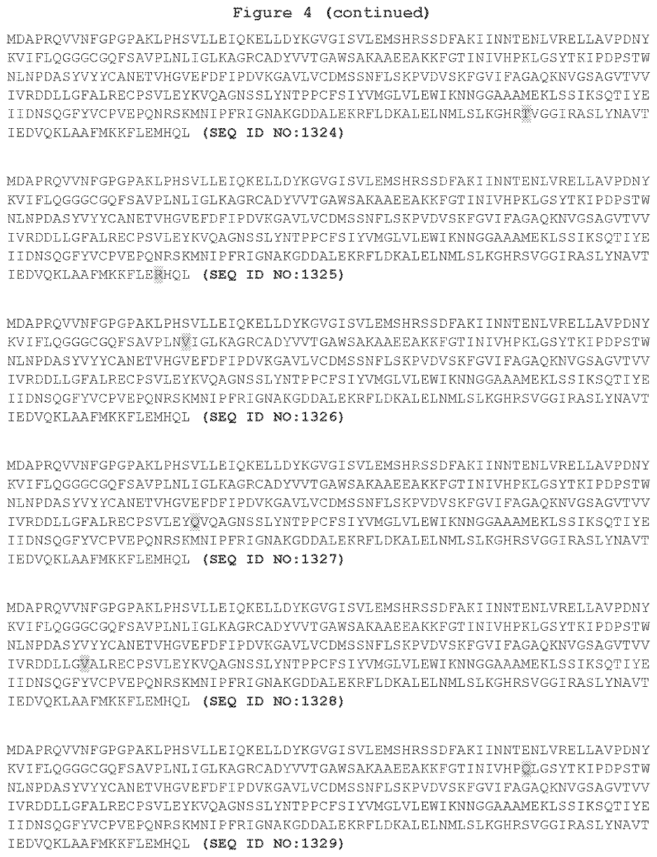

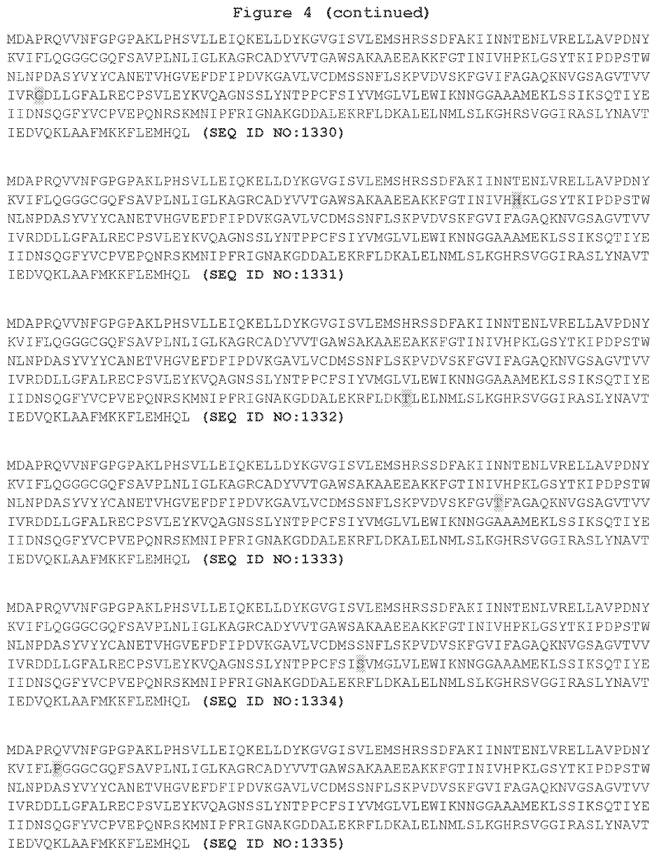

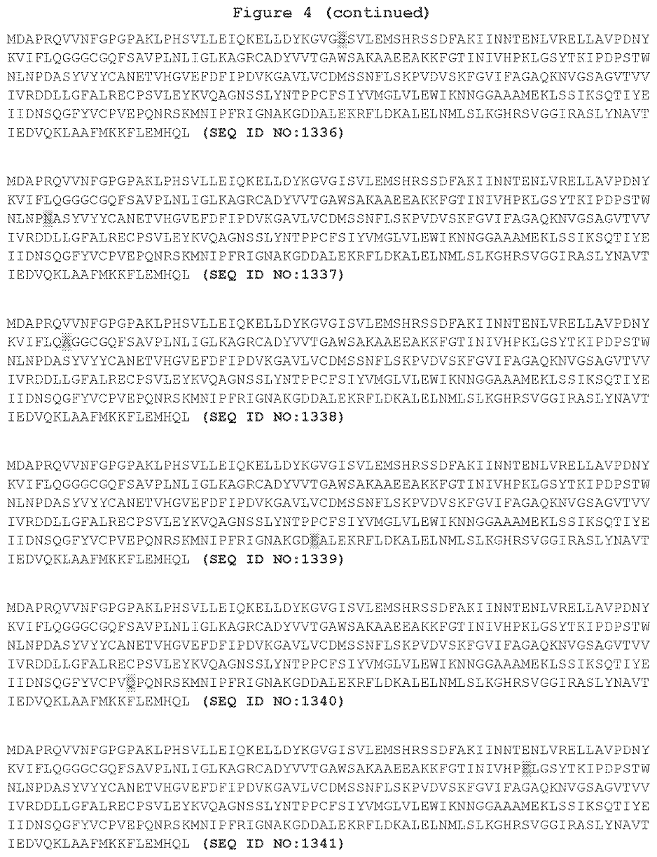

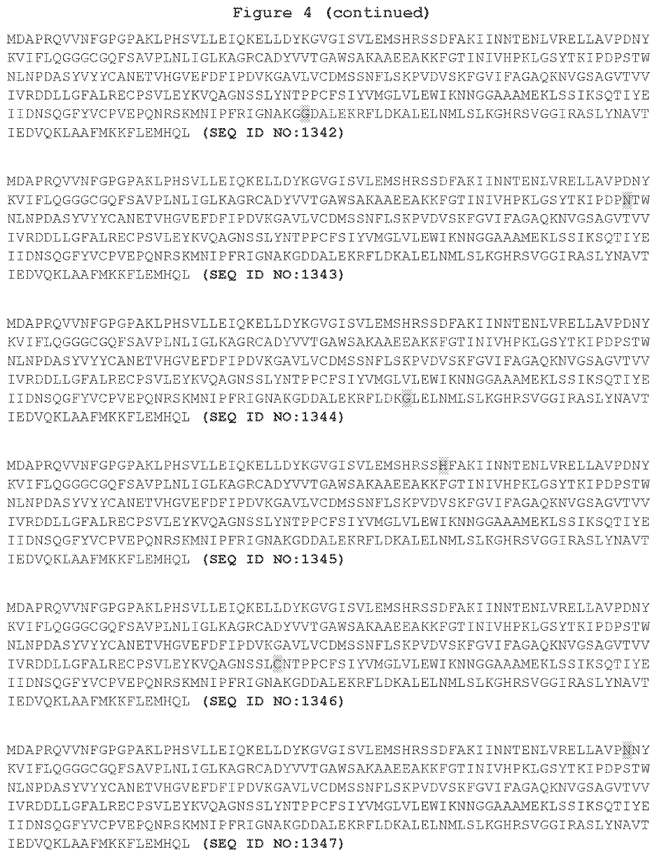

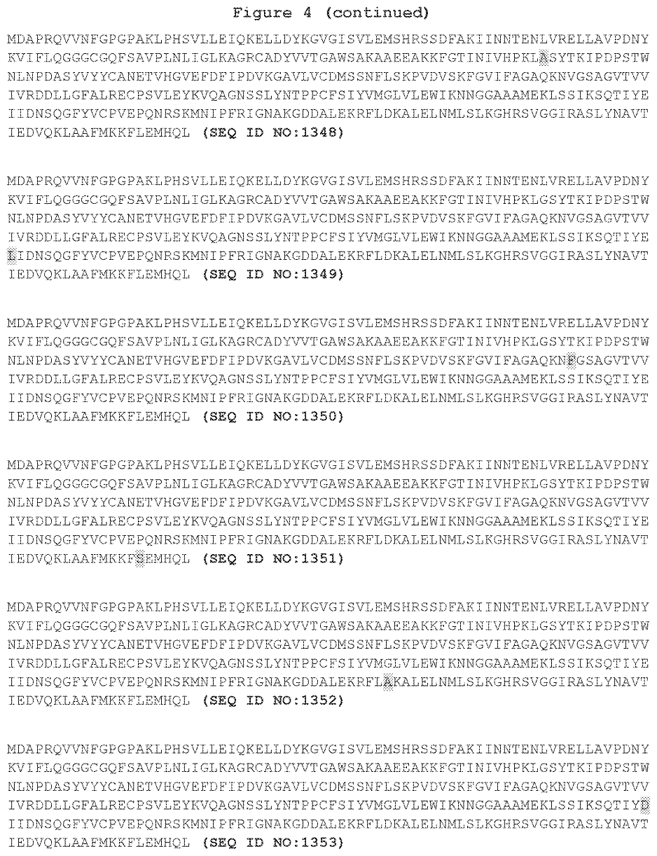

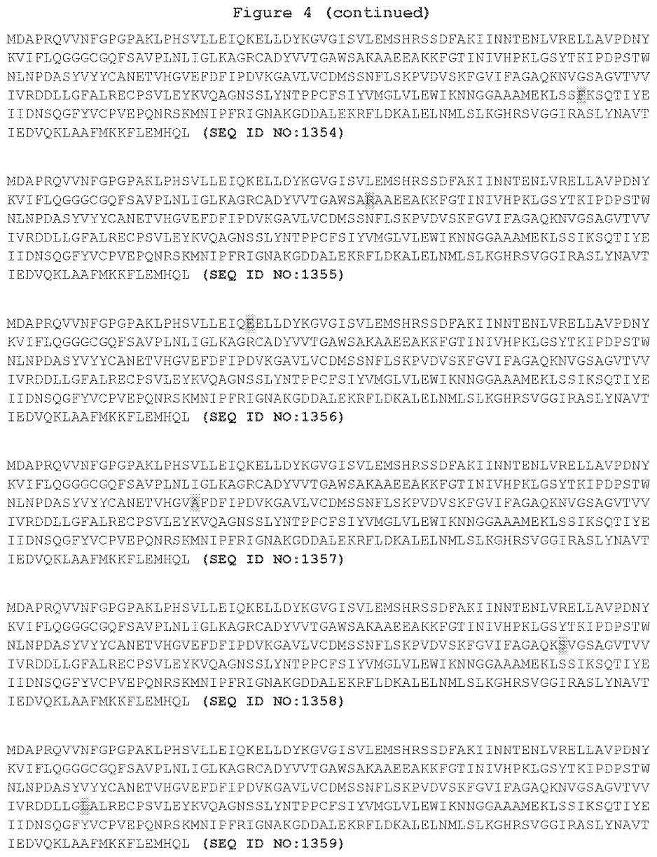

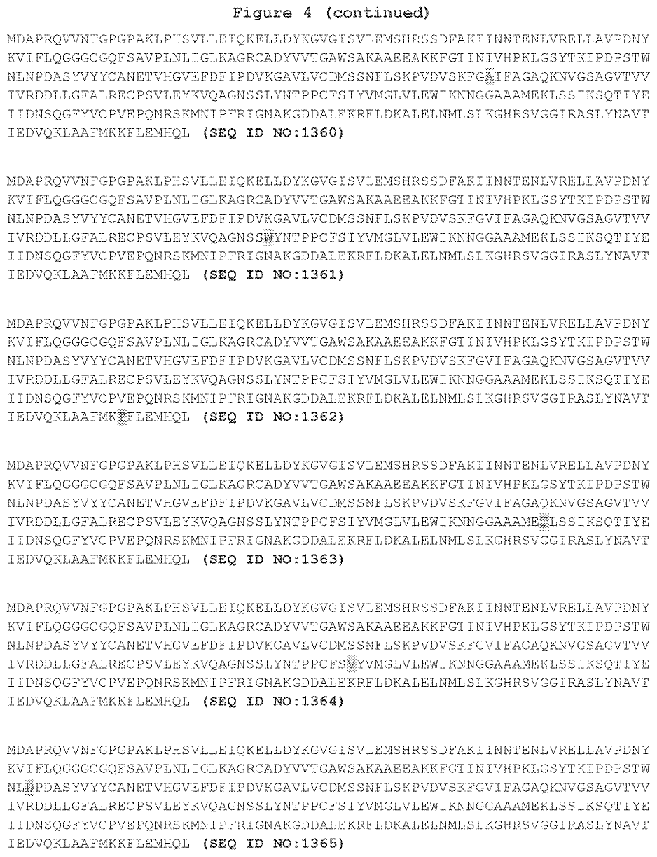

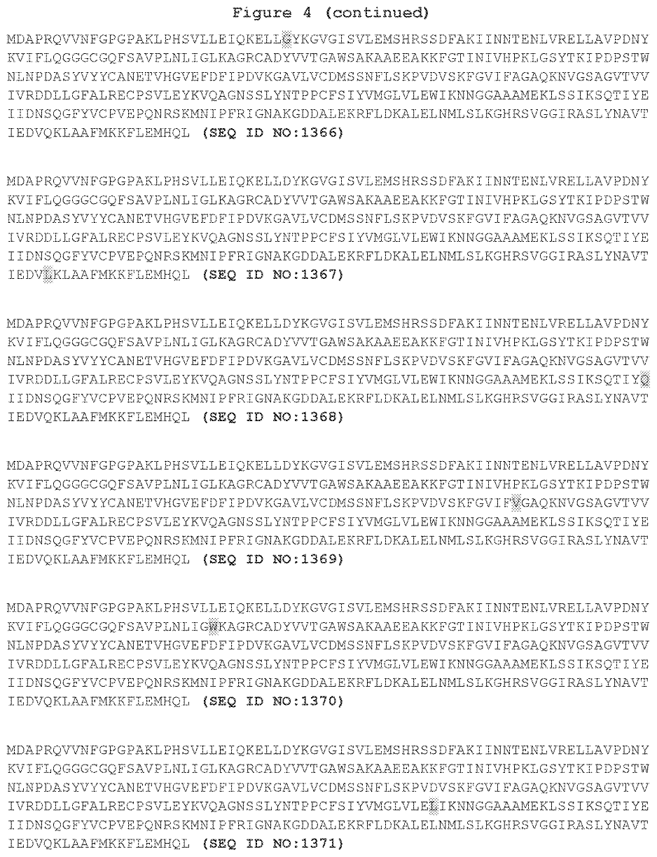

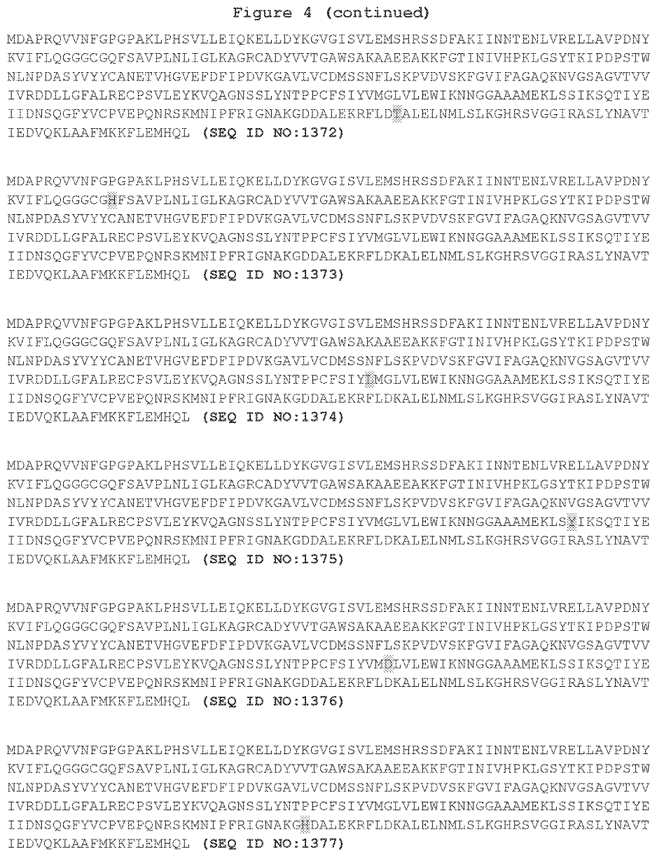

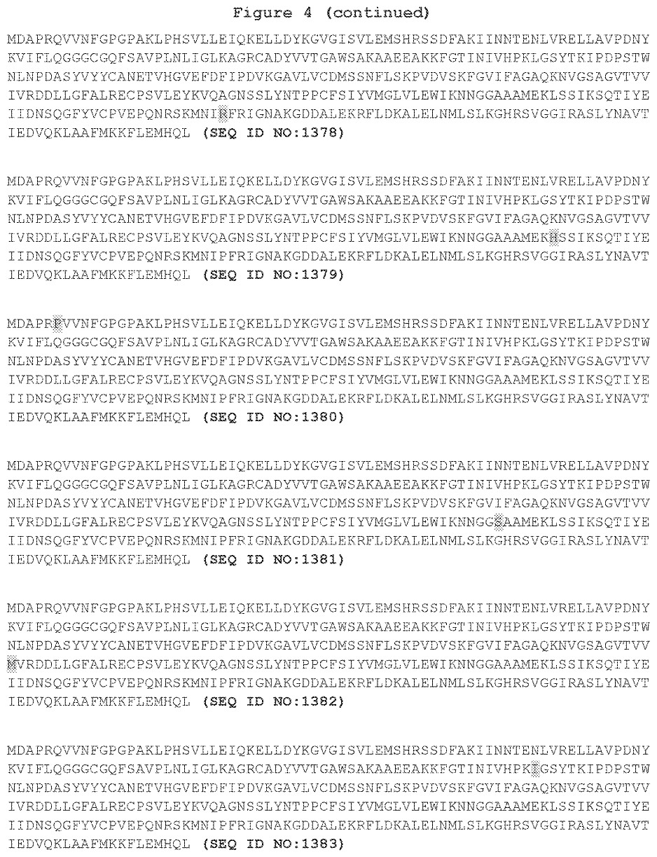

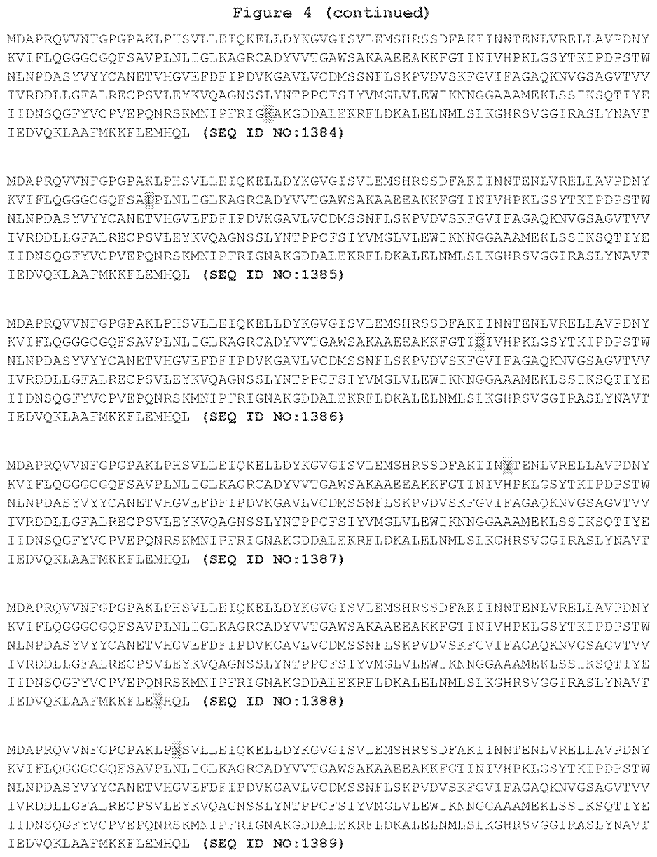

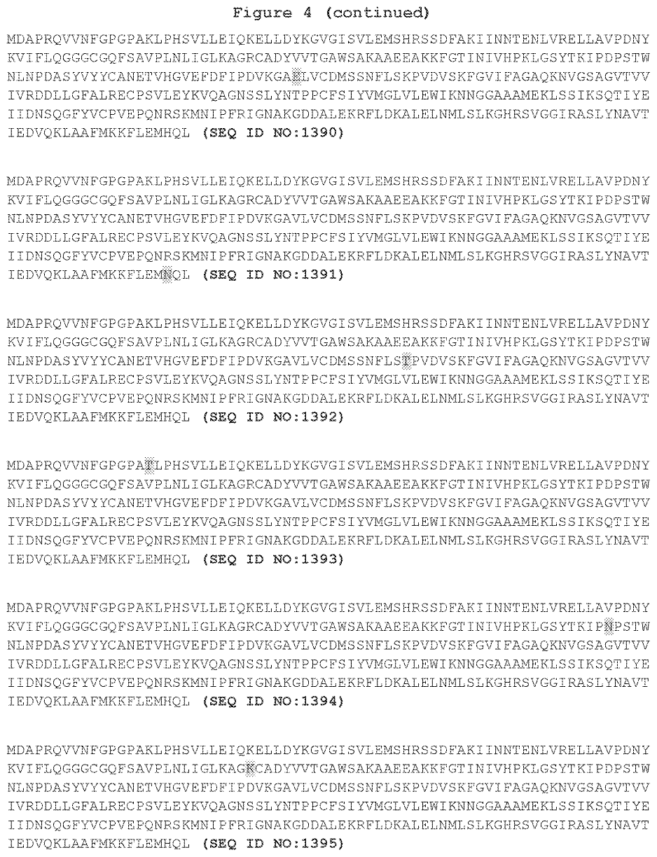

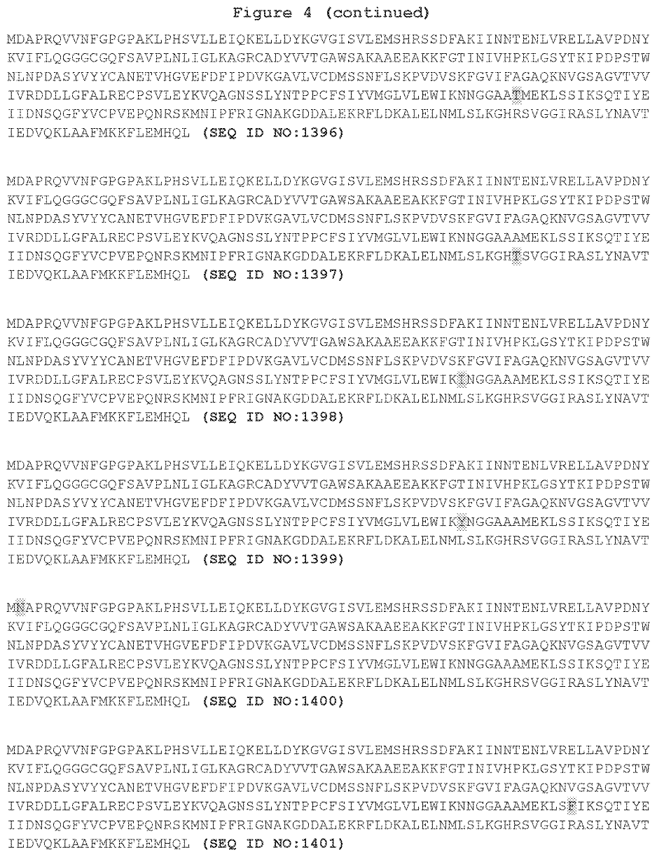

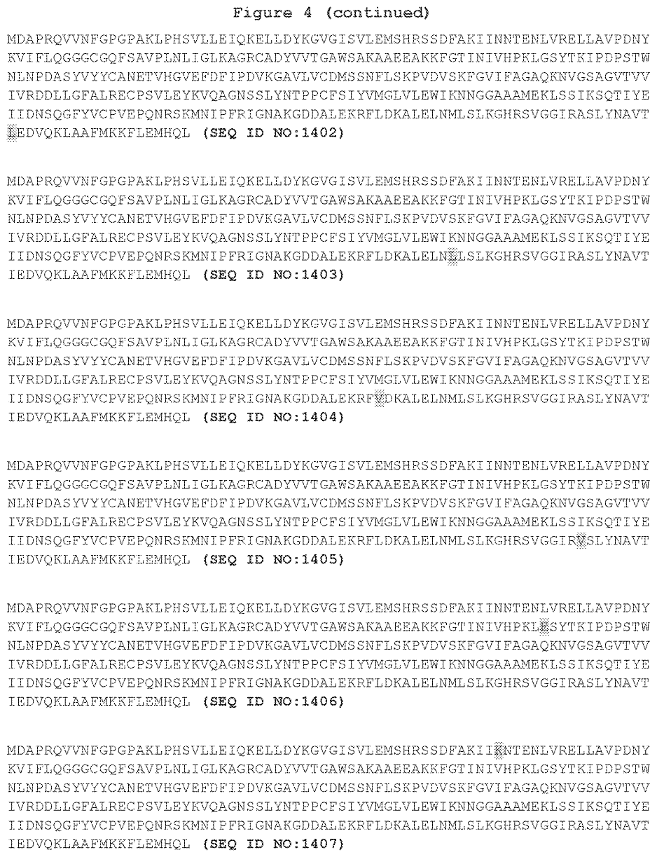

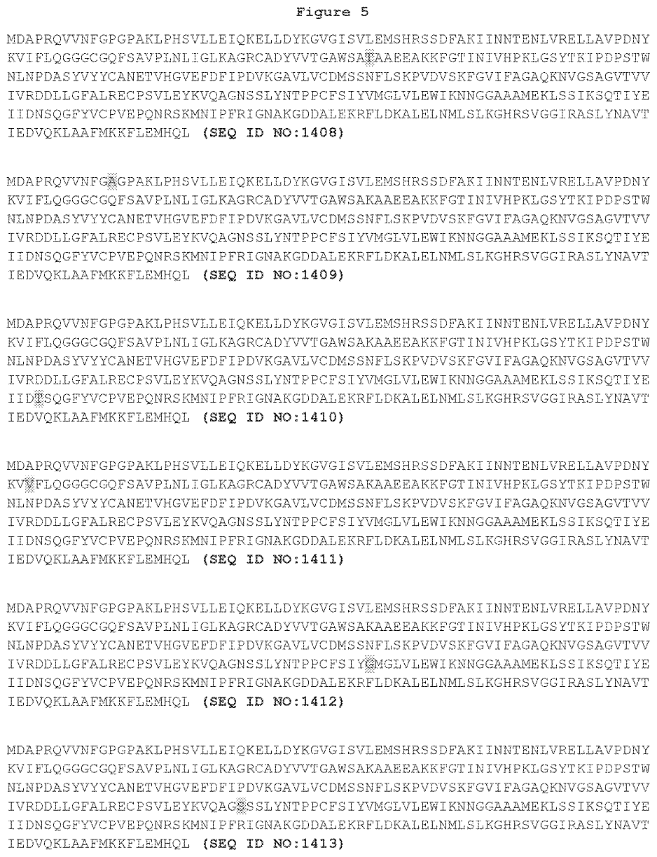

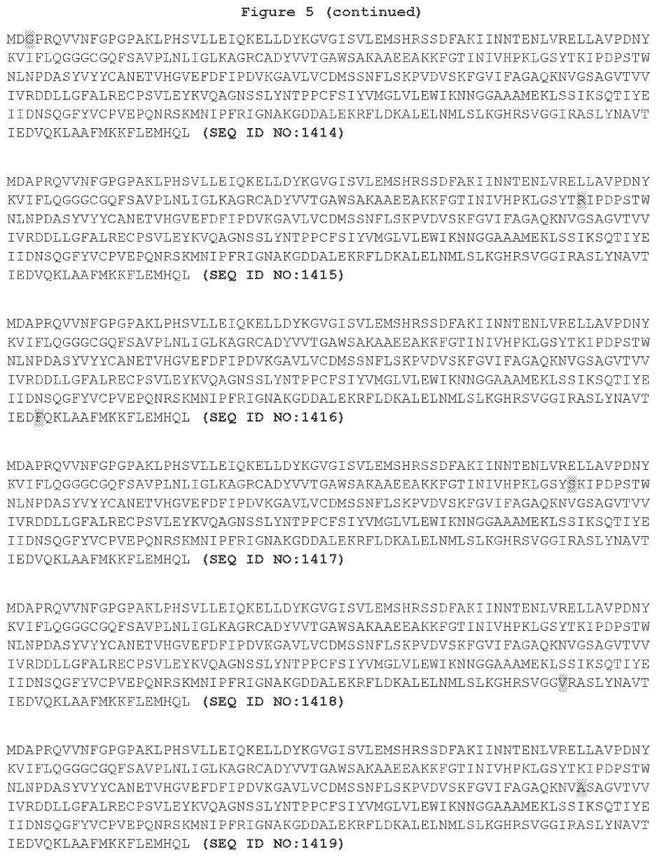

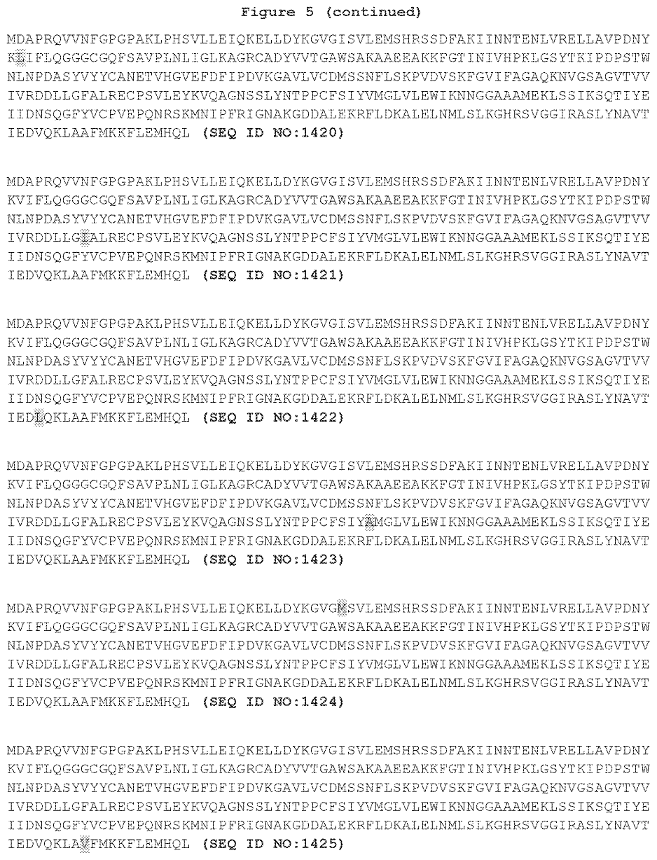

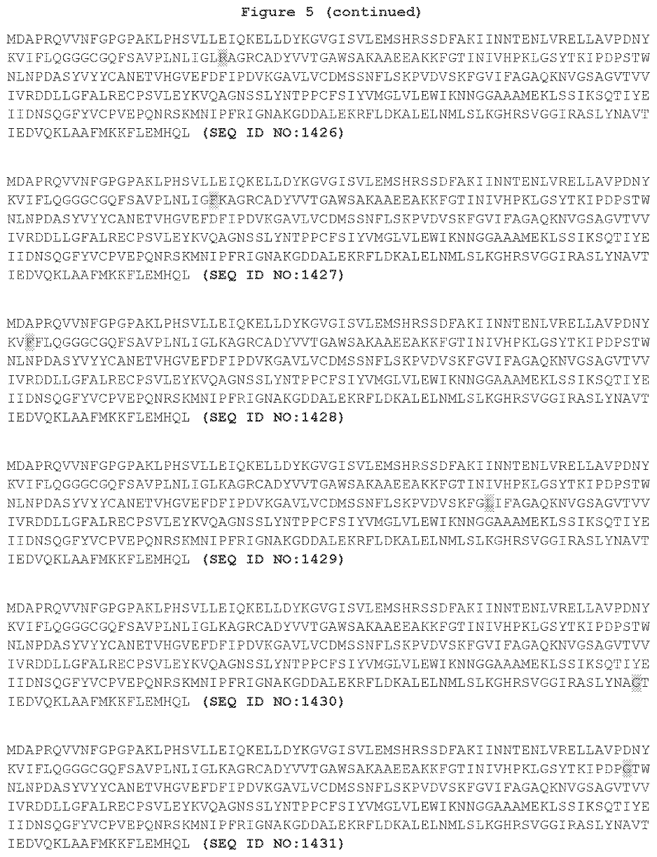

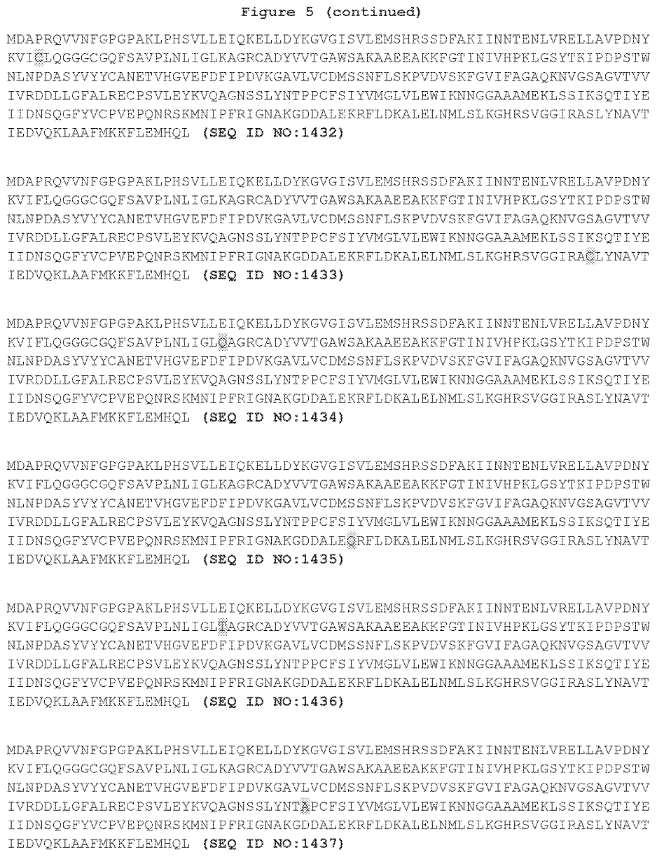

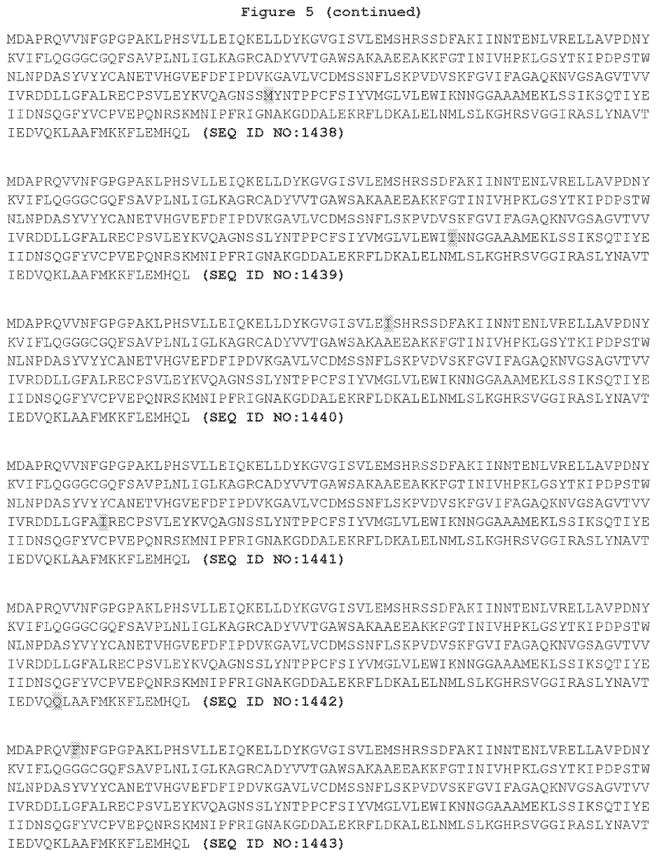

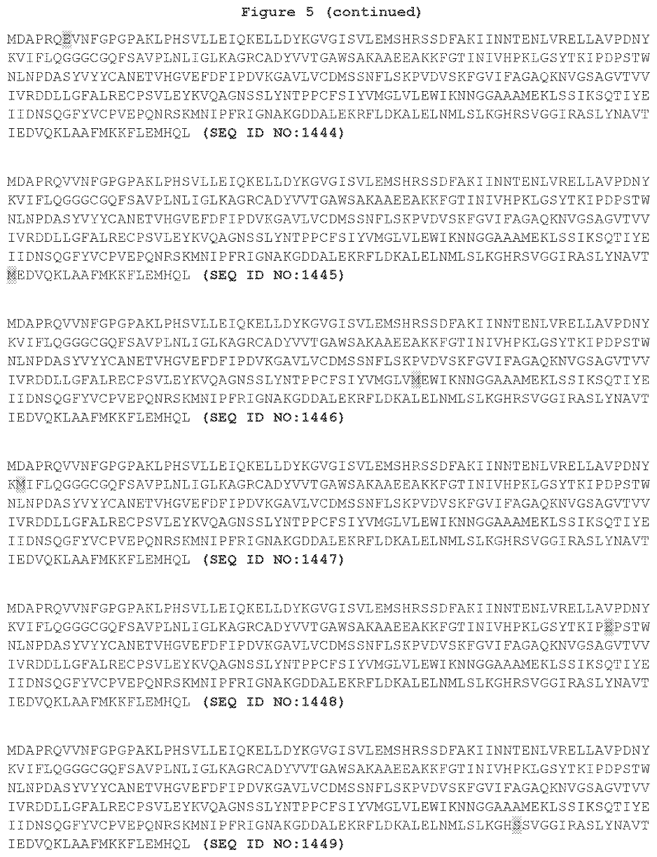

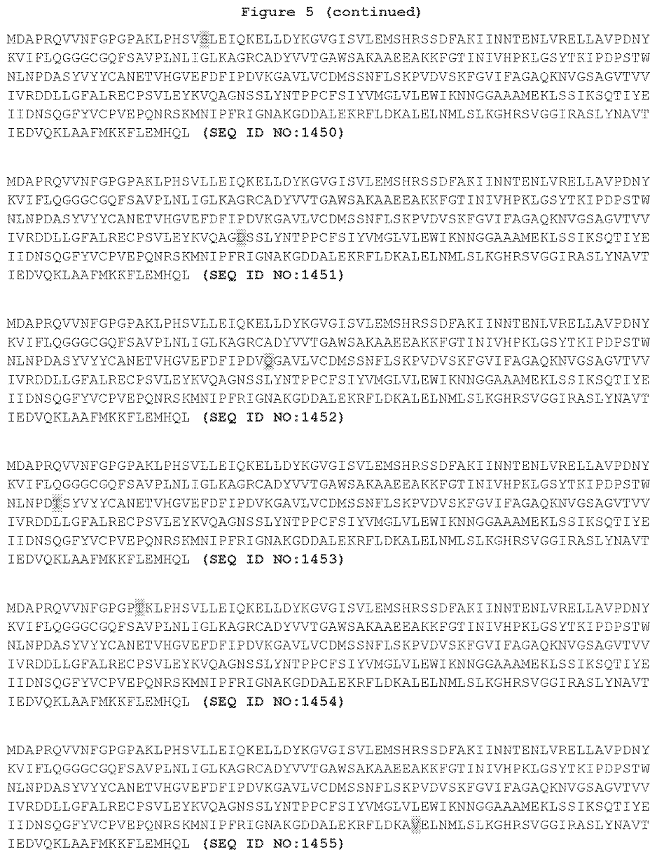

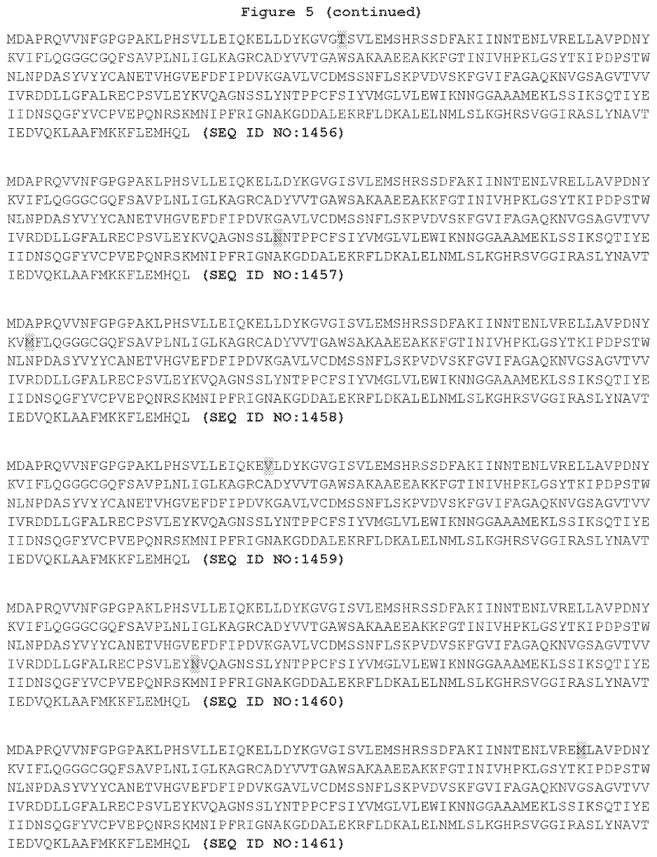

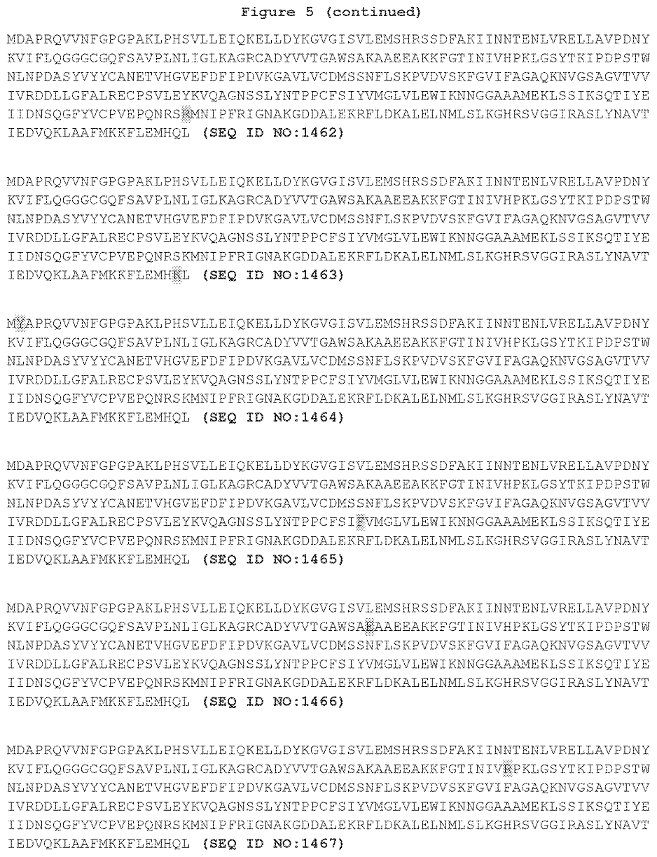

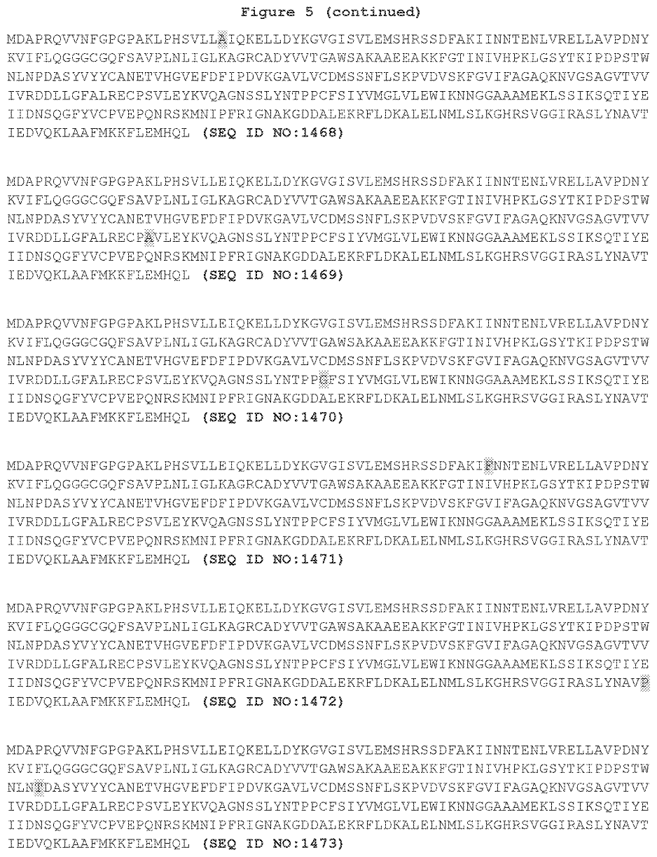

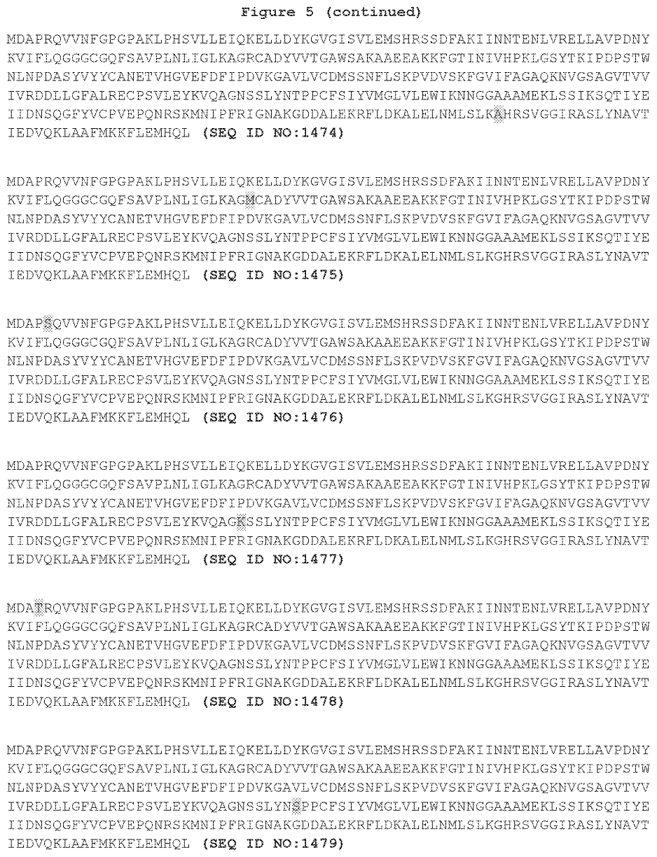

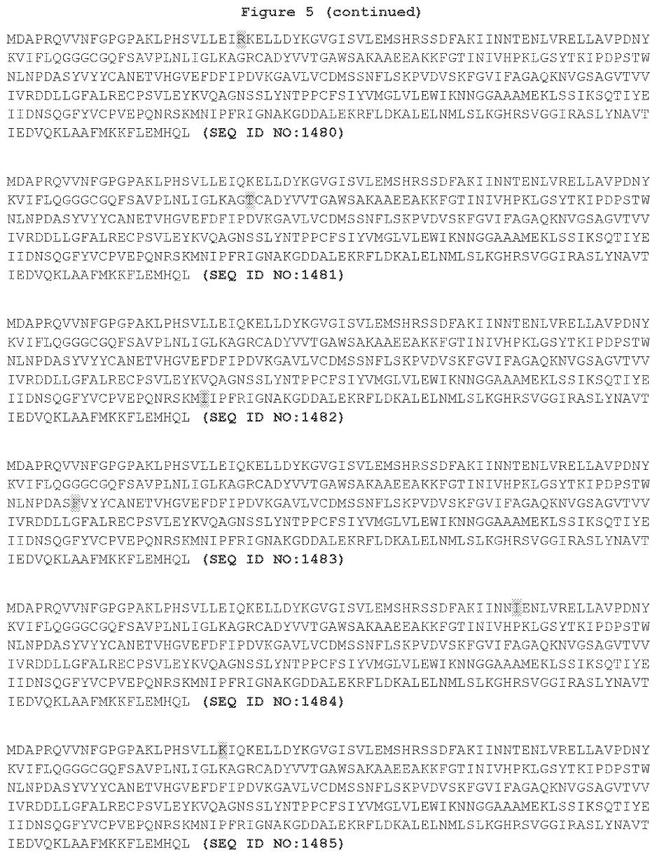

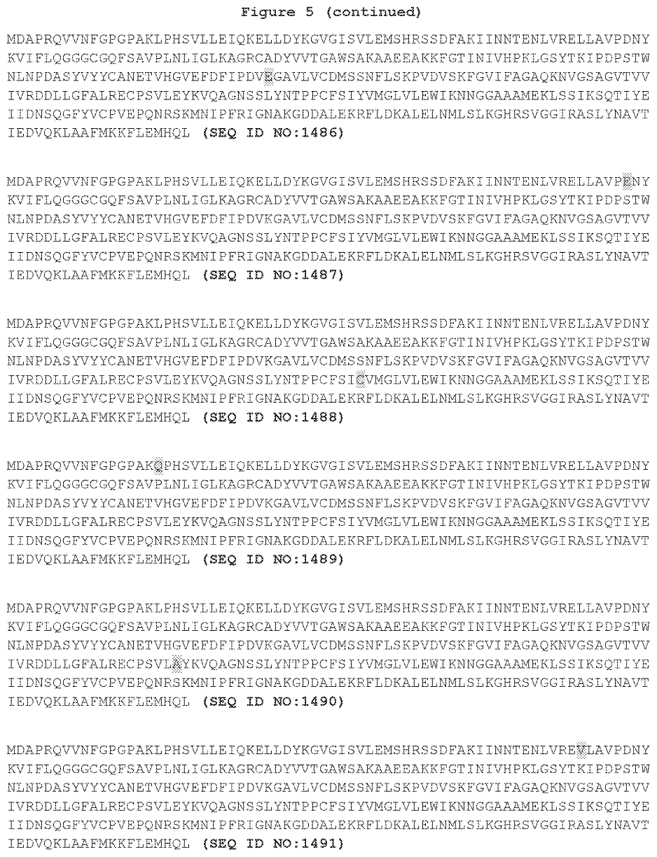

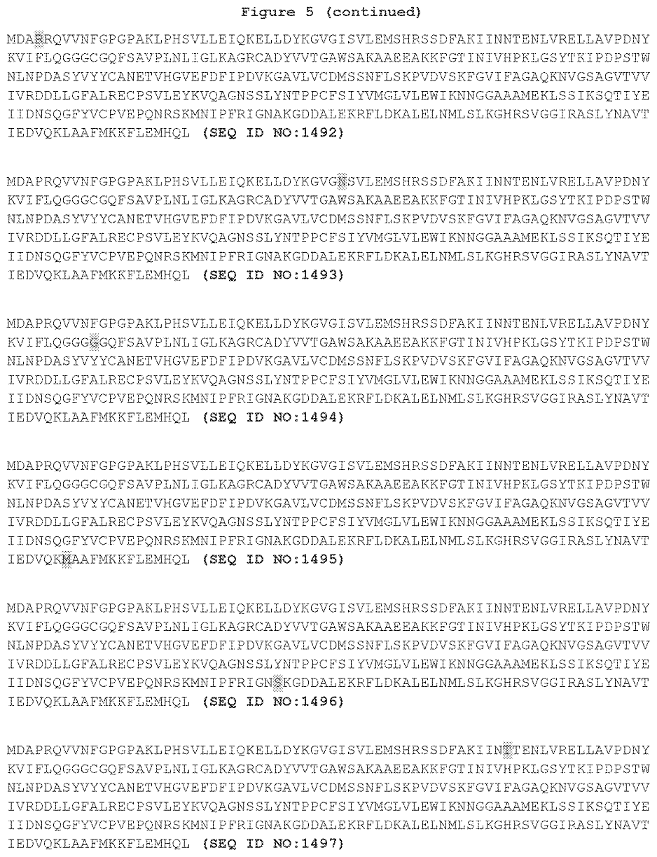

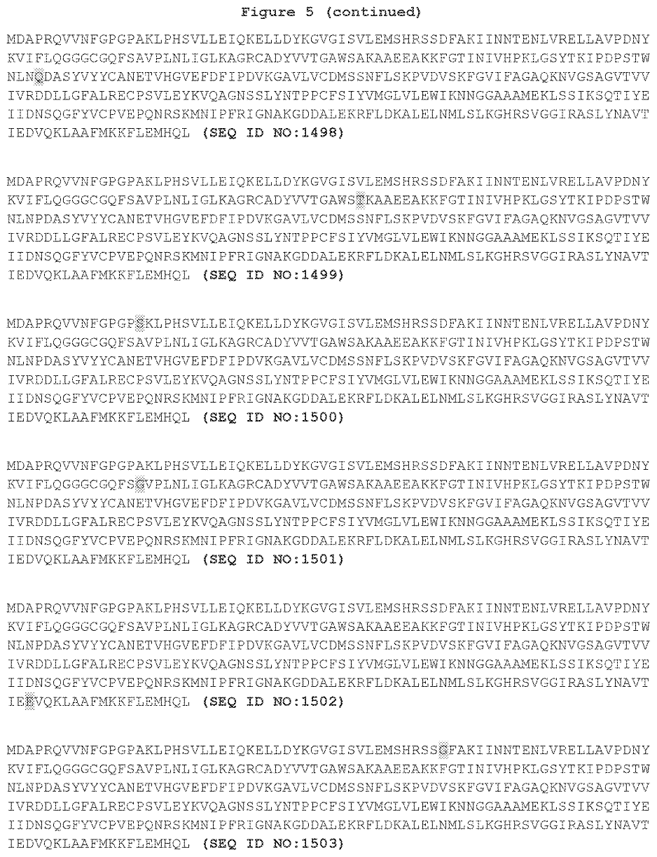

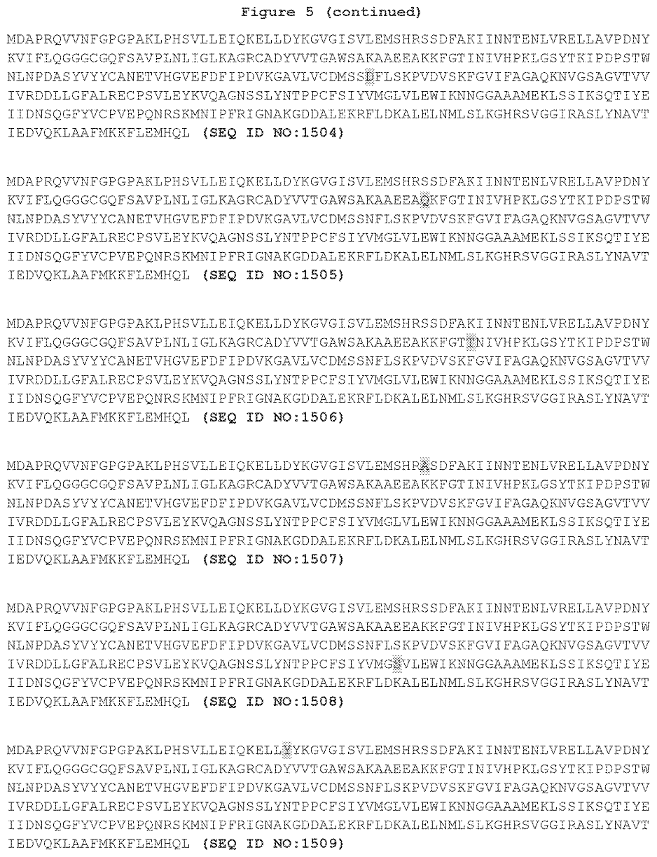

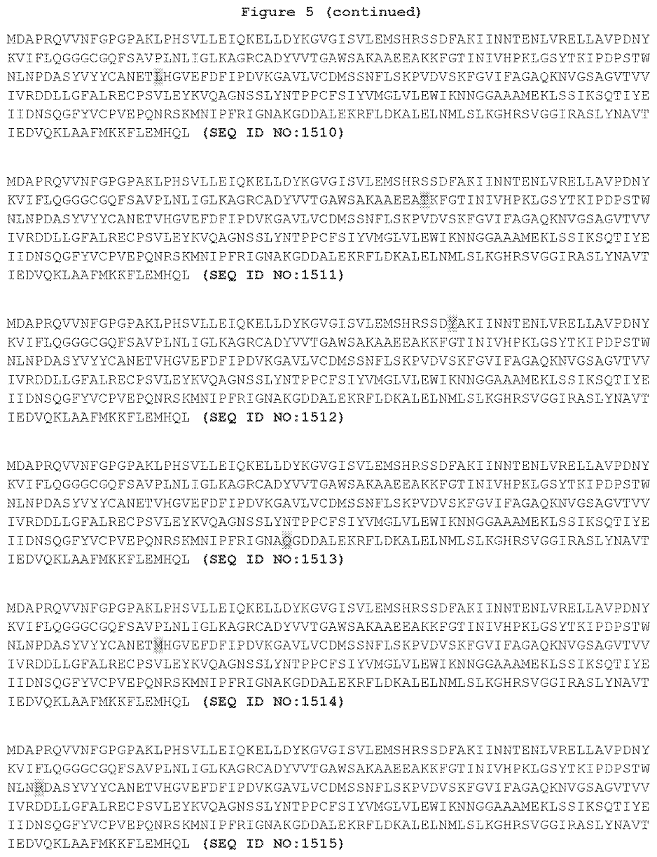

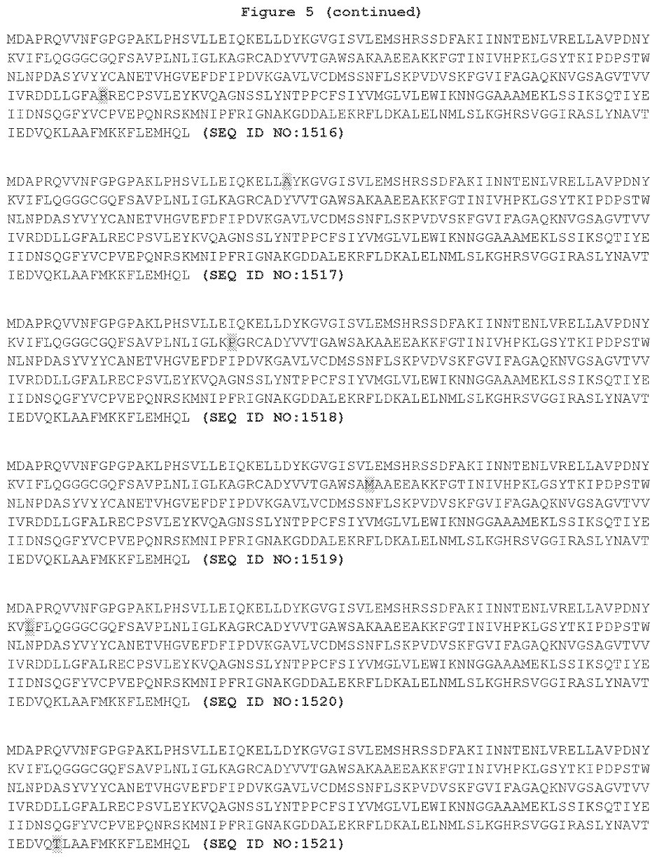

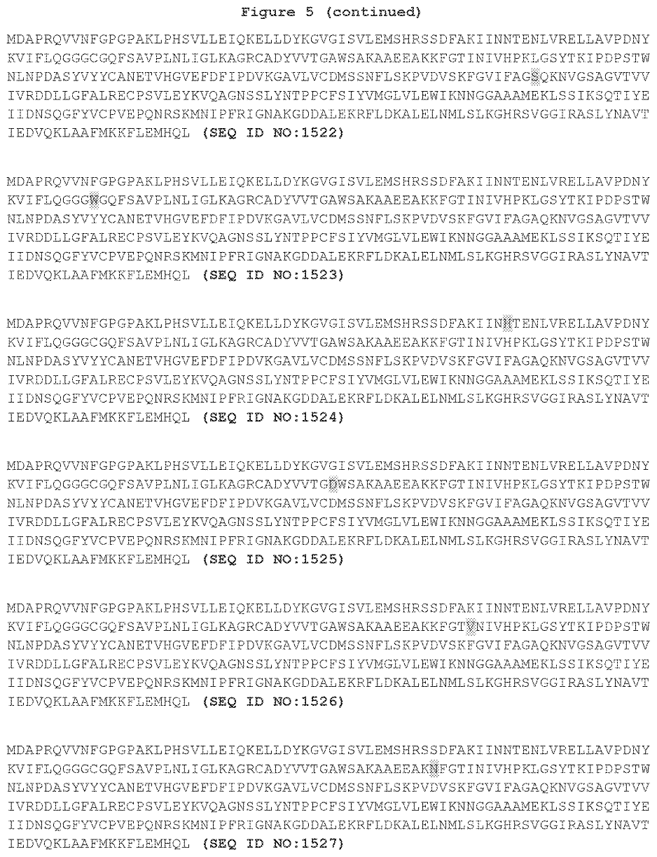

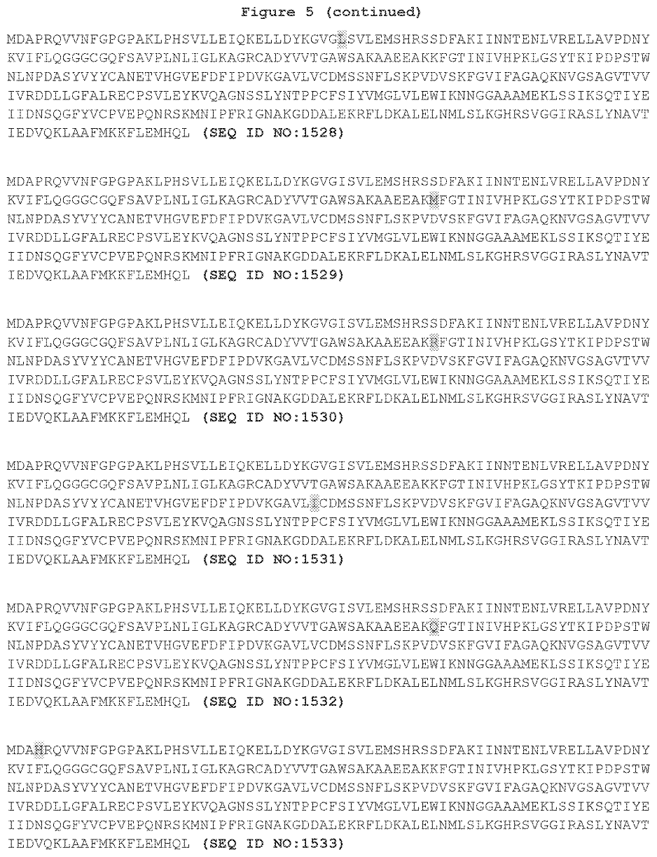

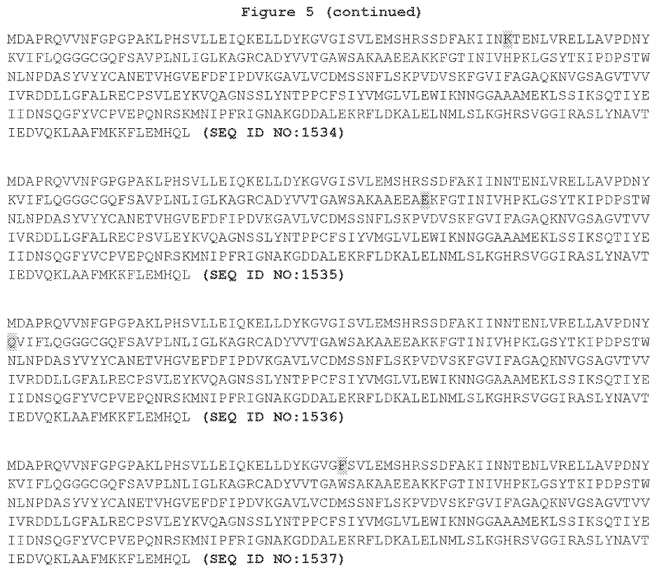

For the amino acid substitution mutants, the sequences of SEQ ID NOS: 4-1537 as depicted in FIGS. 4 and 5 were used.

For expression in yeast, the yPSAT1 gene was placed under the control of the orthologous yeast gene's regulatory elements (SER1 promoter and SER1 terminator). The PSAT1 codon_optimized integrating plasmid (GenBank accession no. MN654100) is further described in detail:
1-6097=Cloning vector pAS19_yPSAT1
1-1336=pUC19 sequence
1337-1577=sequence derived from S. cerevisiae sequence 3′ of the SER1 (YOR184W) terminator
1578-1839=terminator sequence derived from A. gossypii TEF
1840-2649=KanR aminoglycoside 3′-phosphotransferase
2650-3083=promoter sequence derived from A. gossypii TEF.
3084-3546=terminator sequence derived from S. cerevisiae 3′ of the SER1 (YOR184W)
stop codon 3547-4659=yPSAT1 derived from Homo sapiens phosphoserine aminotransferase, codon optimized for expression in S. cerevisiae
4660-4755=derived from S. cerevisiae 5′ of SER1 (YOR184W) start codon
4756-6097=pUC19 sequence

### Example 2. Assay for Identification of High Functional and Stable PSAT1 Protein Variants

Phosphoserine aminotransferase activity is required for yeast to grow in the absence of exogenously supplied serine. Colony growth on minimal medium lacking serine is a quantitative measurement of enzyme function that is amenable to high throughput analysis. A library of all SNP-accessible amino acid substitution mutations across the length of the human PSAT1 protein was introduced. Following integration into the yeast genome in single copy, the growth of yeast strains harboring each PSAT1 variant was quantified from assaying growth on medium lacking serine using the protocol described herein. Yeast cells expressing wild type PSAT1 or variant proteins were grown to saturation in rich liquid medium. The yeast cells were then pinned onto solid medium utilizing glycerol as the carbon source (YPG: 1% yeast extract, 2% peptone, 2% glycerol, 2% Bacto Agar) to remove any respiratory-deficient yeast cells. Yeast cells were then pinned back into rich liquid medium (YPD: 1% yeast extract, 2% peptone, 2% glucose) and grown to saturation. Yeast cells were then pinned in replicate (n=3-6) onto solid minimal medium (SD: 1.7 g/L Bacto Yeast Nitrogen Base without amino acids or ammonium sulfate, 0.5% ammonium sulfate, 2% glucose, 2% Bacto Agar), which lacks serine, and grown for 3 days at 30 °C. Pinning was performed using a Biomek i7 robot outfitted with a V&P 96-pin head. The DNA sequences of the expression constructs present in each strain were determined and strains harboring expression constructs with secondary mutations (in addition to the intended amino acid substitution mutation) were removed from further consideration. The growth values represent the median values from at least one (and in most cases more than one) independently derived strain, each of which was assayed at least three times. The standard error reflects the error associated with these technical and biological replicates.

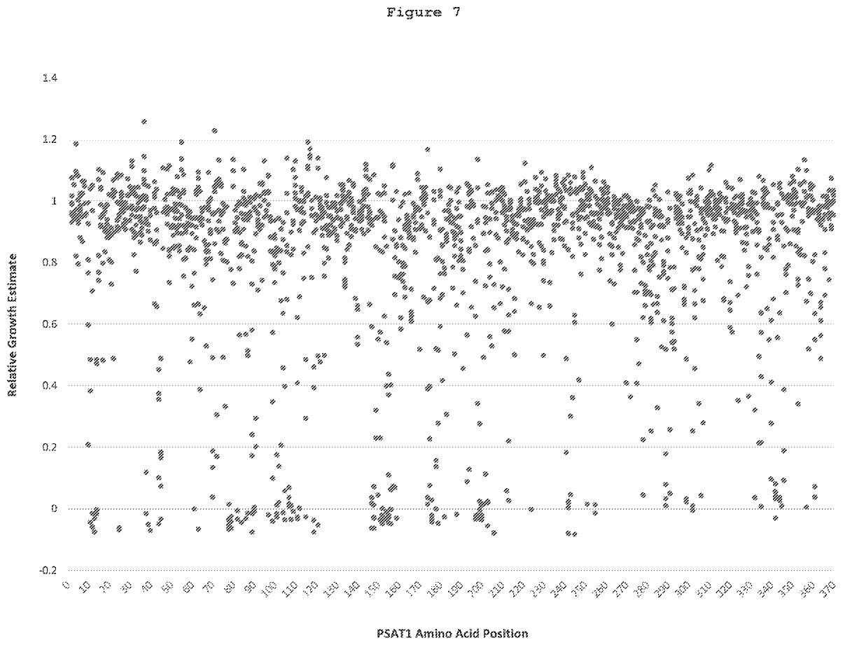

Growth score was used as a measure of functional impact of the variants. The growth of 95% (1823 variants out of 1914) of the total possible SNP-accessible variants listed in Table 1 were quantitated. The growth values are plotted as points on a scatter plot ordered by their amino acid position as shown in FIG. 7. Functional scores below 0.05 in the assay represent alleles with extremely low activity and thus strains showed little growth. Approximately 3% of variants displayed growth values in this range of the assay. Approximately 74% of variants fell in the range between 0.05 and the value of the wildtype protein (1.0). The remaining 23% of variants exhibited growth values greater than that conferred by the wildtype protein.

The results of the assay are depicted as median normalized growth value and are shown in Table 1. The protein variants that exhibited a growth score of greater than 1.05 of the wild-type SEQ ID NO: 1 are listed in Table 2. The protein variants that exhibited a growth score of less than 0.99 (e.g., 0.98, 0.95, 0.9, 0.85, 0.8, 0.75, 0.7, 0.6, or lower), of the wild-type SEQ ID NO: 1 are listed in Table 3. Table 1 includes protein variants listed in Tables 2 and 3.

TABLE 1

| SEQ | | | | | SEQ | | | | |
|---|---|---|---|---|---|---|---|---|---|
| ID | AA | | Growth | | ID | AA | | Growth | |
| NO. | Position | Variant | Estimate | S_Error | NO. | Position | Variant | Estimate | S_Error |
| 4 | 245 | C245R | −0.082328909 | 0.002800309 | 42 | 108 | S108P | −0.033937177 | 0.012220522 |
| 5 | 242 | T242M | −0.080047746 | 0.004149365 | 43 | 197 | G197D | −0.033040395 | 0.012276394 |
| 5 | 206 | G206R | −0.078593761 | 0.004638939 | 44 | 82 | Q82R | −0.032856792 | 0.007771615 |
| 7 | 89 | N89Y | −0.076024013 | 0.010489297 | 45 | 45 | R45S | −0.032410529 | 0.008708271 |
| 8 | 119 | G119E | −0.074937864 | 0.010896115 | 46 | 78 | G78C | −0.032387435 | 0.017418183 |
| 9 | 13 | G13R | −0.074853551 | 0.007726466 | 47 | 111 | A111P | −0.03188857 | 0.008730803 |
| 10 | 40 | L40R | −0.070063395 | 0.005510421 | 48 | 86 | V86D | −0.031614093 | 0.01748482 |
| 11 | 25 | I25K | −0.068982638 | 0.007261152 | 49 | 154 | N154Y | −0.031591026 | 0.006182799 |
| 12 | 78 | G78V | −0.067526096 | 0.009120766 | 50 | 13 | G13S | −0.031082139 | 0.008765275 |
| 13 | 63 | L63S | −0.065644994 | 0.013282044 | 51 | 199 | Q199P | −0.031025408 | 0.012399223 |
| 14 | 78 | G78D | −0.064776836 | 0.009510026 | 52 | 148 | Y148H | −0.030834835 | 0.012410653 |
| 15 | 148 | Y148N | −0.063441146 | 0.00968306 | 53 | 342 | R342Q | −0.030718572 | 0.008780684 |
| 16 | 79 | G79R | −0.06339475 | 0.005594851 | 54 | 159 | G159R | −0.030177022 | 0.012449865 |
| 17 | 147 | S147Y | −0.062922941 | 0.007959346 | 55 | 200 | K200Q | −0.028624921 | 0.012540944 |
| 18 | 25 | I25R | −0.060336391 | 0.014216401 | 56 | 148 | Y148D | −0.028175623 | 0.017772252 |
| 19 | 12 | P12L | −0.058712175 | 0.010231123 | 57 | 200 | K200T | −0.02738975 | 0.017836024 |
| 20 | 203 | G203R | −0.055514227 | 0.00862007 | 58 | 155 | E155V | −0.027151588 | 0.005951959 |
| 21 | 121 | I121K | −0.052304855 | 0.015352926 | 59 | 115 | A115D | −0.026906113 | 0.008937588 |
| 22 | 78 | G78A | −0.051541489 | 0.010924077 | 60 | 182 | L182R | −0.026329136 | 0.012672131 |
| 23 | 39 | V39D | −0.051222679 | 0.006927088 | 61 | 111 | A111D | −0.026209767 | 0.008965372 |
| 24 | 44 | H44Y | −0.049616696 | 0.01108933 | 62 | 101 | Y101D | −0.02595513 | 0.017950709 |
| 25 | 152 | C152F | −0.048233654 | 0.011203662 | 63 | 79 | G79E | −0.024949452 | 0.006010122 |
| 26 | 156 | T156M | −0.047889982 | 0.009170677 | 64 | 200 | K200R | −0.024920487 | 0.012750651 |
| 27 | 179 | S179L | −0.047252936 | 0.011282662 | 65 | 197 | G197R | −0.022771772 | 0.010506467 |
| 28 | 155 | E155Q | −0.044703109 | 0.00489603 | 66 | 152 | C152Y | −0.021190719 | 0.012951831 |
| 29 | 84 | S84R | −0.044655405 | 0.009376991 | 67 | 176 | D176N | −0.020624056 | 0.00821034 |
| 30 | 11 | G11R | −0.043414428 | 0.006684315 | 68 | 155 | E155K | −0.020189244 | 0.006502237 |
| 31 | 176 | D176H | −0.042784012 | 0.011623159 | 69 | 198 | A198D | −0.019755153 | 0.013026825 |
| 32 | 79 | G79W | −0.040856332 | 0.005015548 | 70 | 200 | K200M | −0.019697931 | 0.006514983 |
| 33 | 99 | A99D | −0.039340649 | 0.009689034 | 71 | 97 | R97G | −0.019513188 | 0.018440369 |
| 34 | 78 | G78R | −0.037862237 | 0.016922998 | 72 | 188 | V188D | −0.017887665 | 0.013122486 |
| 35 | 152 | C152R | −0.037857259 | 0.011966819 | 73 | 14 | P14T | −0.016453347 | 0.018659901 |
| 36 | 119 | G119R | −0.036858248 | 0.017017083 | 74 | 12 | P12R | −0.015713772 | 0.010803303 |
| 37 | 176 | D176A | −0.036050332 | 0.006977958 | 75 | 89 | N89K | −0.015207742 | 0.010823666 |
| 38 | 105 | G105V | −0.035764596 | 0.009883154 | 76 | 38 | S38R | −0.015090776 | 0.010828357 |
| 39 | 200 | K200N | −0.035476397 | 0.012122799 | 77 | 101 | Y101N | −0.015080221 | 0.009378011 |
| 40 | 151 | Y151D | −0.035371981 | 0.006065061 | 78 | 87 | P87R | −0.014097394 | 0.010867951 |
| 41 | 176 | D176Y | −0.034300397 | 0.012197673 | 79 | 13 | G13C | −0.01406565 | 0.007685717 |
| 80 | 255 | L255P | −0.013439858 | 0.010893923 | 121 | 203 | G203V | 0.024015376 | 0.012138399 |
| 81 | 104 | T104P | −0.012901704 | 0.013368129 | 122 | 242 | T242K | 0.024221862 | 0.014873595 |
| 82 | 154 | N154D | −0.010154661 | 0.013497883 | 123 | 344 | S344Y | 0.026520322 | 0.014952959 |
| 83 | 109 | A109P | −0.008011609 | 0.01922845 | 124 | 213 | R213P | 0.026737935 | 0.014960408 |
| 84 | 90 | L90F | −0.007327339 | 0.019272381 | 125 | 176 | D176E | 0.027877022 | 0.012246853 |
| 85 | 82 | Q82P | −0.006096347 | 0.013682989 | 126 | 299 | S299I | 0.032739222 | 0.01516159 |
| 86 | 302 | N302Y | −0.004936069 | 0.013734555 | 127 | 342 | R342W | 0.032945749 | 0.015168373 |
| 87 | 14 | P14L | −0.004270715 | 0.009732525 | 128 | 289 | Y2895 | 0.032981137 | 0.02145286 |
| 88 | 14 | P14H | −0.003079532 | 0.011280618 | 129 | 174 | V174F | 0.035960653 | 0.010795071 |
| 89 | 199 | Q199K | −0.002457549 | 0.013842805 | 130 | 107 | W107L | 0.035961132 | 0.021589805 |
| 90 | 156 | T156P | −0.002365385 | 0.005652926 | 131 | 147 | S147P | 0.03608325 | 0.009657955 |
| 91 | 156 | I56R | 0.001642863 | 0.008012374 | 132 | 332 | L332W | 0.036107153 | 0.00881696 |
| 92 | 224 | C224W | −0.001438268 | 0.01963861 | 133 | 344 | S344F | 0.03617341 | 0.012470594 |
| 93 | 61 | R61P | −0.000806458 | 0.019676705 | 134 | 361 | M361R | 0.038060211 | 0.015333512 |
| 94 | 178 | S178P | −0.000281397 | 0.009854098 | 135 | 70 | Y70D | 0.03831843 | 0.021696318 |
| 95 | 152 | C152W | 0.001299128 | 0.008084169 | 136 | 342 | R342P | 0.040753423 | 0.012589137 |
| 96 | 112 | A112P | 0.002333031 | 0.011467791 | 137 | 306 | R306P | 0.04292549 | 0.015485809 |
| 97 | 242 | T242P | 0.003618397 | 0.014097941 | 138 | 278 | I278N | 0.044085481 | 0.012673326 |
| 98 | 357 | L357R | 0.004774341 | 0.014144944 | 139 | 150 | Y150N | 0.044834721 | 0.015544367 |
| 99 | 90 | L90R | 0.00510769 | 0.014158412 | 140 | 243 | P243R | 0.045547102 | 0.015566048 |
| 100 | 335 | H335D | 0.007289441 | 0.014245627 | 141 | 291 | C291F | 0.049659574 | 0.022188048 |
| 101 | 302 | N302K | 0.009110789 | 0.010123817 | 142 | 342 | R342L | 0.056043592 | 0.010040929 |
| 102 | 107 | W107R | 0.009413555 | 0.009062484 | 143 | 212 | V212D | 0.058345144 | 0.022543683 |
| 103 | 345 | L345R | 0.009711734 | 0.009069816 | 144 | 105 | G105A | 0.058922696 | 0.010092704 |
| 104 | 289 | Y289D | 0.010992319 | 0.010175338 | 145 | 156 | T156A | 0.062605347 | 0.016060154 |
| 105 | 108 | S108L | 0.011087689 | 0.020355821 | 146 | 158 | H158P | 0.068873742 | 0.011477323 |
| 106 | 148 | Y148S | 0.011829232 | 0.011775635 | 147 | 107 | W107S | 0.069223214 | 0.016240158 |
| 107 | 101 | Y1015 | 0.011985856 | 0.014428114 | 148 | 148 | Y1480 | 0.071870983 | 0.023066129 |
| 108 | 200 | K200E | 0.01201013 | 0.020405729 | 149 | 361 | M361K | 0.072574696 | 0.013332906 |
| 109 | 255 | L255R | 0.013416034 | 0.020481098 | 150 | 45 | R45M | 0.073107727 | 0.009436446 |
| 110 | 174 | V174D | 0.014070524 | 0.014506948 | 151 | 157 | V157E | 0.073138611 | 0.008172935 |
| 111 | 78 | G785 | 0.014348858 | 0.009181622 | 152 | 175 | C175W | 0.076466775 | 0.016430571 |
| 112 | 251 | M251R | 0.014371901 | 0.014518239 | 153 | 289 | Y289N | 0.079028399 | 0.023328835 |
| 113 | 201 | N201Y | 0.016482842 | 0.011918087 | 154 | 342 | R342G | 0.081698174 | 0.023424595 |
| 114 | 147 | S147F | 0.017402604 | 0.011945647 | 155 | 193 | V193E | 0.088728541 | 0.016738611 |
| 115 | 107 | W107C | 0.017591838 | 0.010350139 | 156 | 346 | Y346N | 0.092947628 | 0.013750862 |
| 116 | 203 | G203D | 0.020064779 | 0.020826725 | 157 | 340 | G340R | 0.096645899 | 0.008466079 |
| 117 | 242 | T242R | 0.020792734 | 0.020863538 | 158 | 99 | A99P | 0.099456424 | 0.016994866 |
| 118 | 102 | V102E | 0.022084658 | 0.011463014 | 159 | 44 | H44L | 0.100716905 | 0.02407505 |
| 119 | 299 | S299R | 0.022588407 | 0.014816413 | 160 | 155 | E155G | 0.109845778 | 0.006762159 |
| 120 | 199 | Q199L | 0.023921015 | 0.012135712 | 161 | 202 | V202D | 0.112330654 | 0.010935209 |
| 162 | 38 | S38I | 0.118686302 | 0.017427243 | 203 | 285 | S285Y | 0.328555414 | 0.012045231 |
| 163 | 194 | I194N | 0.128245208 | 0.01763076 | 204 | 76 | Q76R | 0.332833444 | 0.020896458 |
| 164 | 70 | Y70N | 0.135126482 | 0.010263642 | 205 | 353 | D353V | 0.340437769 | 0.029679104 |
| 165 | 178 | S178F | 0.137530207 | 0.017821947 | 206 | 305 | F305S | 0.342092143 | 0.017161098 |
| 166 | 102 | V102G | 0.139296116 | 0.025252958 | 207 | 198 | A198P | 0.342572751 | 0.029716556 |
| 167 | 178 | S178Y | 0.158569473 | 0.012895235 | 208 | 99 | A99T | 0.347366056 | 0.029800131 |
| 168 | 45 | R45G | 0.16656049 | 0.013001076 | 209 | 324 | A324P | 0.352421138 | 0.017265164 |
| 169 | 72 | V72G | 0.170212399 | 0.02609055 | 210 | 44 | H44N | 0.356237655 | 0.02118596 |
| 170 | 89 | N89I | 0.173467933 | 0.026174203 | 211 | 244 | P244R | 0.361646356 | 0.030045068 |
| 171 | 101 | Y101C | 0.176154215 | 0.026242631 | 212 | 272 | S272P | 0.364523652 | 0.021285709 |
| 172 | 289 | Y289H | 0.179144051 | 0.011774207 | 213 | 329 | M329R | 0.364882391 | 0.021289997 |
| 173 | 45 | R45K | 0.180217905 | 0.010760393 | 214 | 155 | E15SA | 0.371410186 | 0.008749598 |
| 174 | 241 | N241I | 0.182017486 | 0.026390163 | 215 | 44 | H44P | 0.37484773 | 0.017484995 |
| 175 | 45 | R45T | 0.185541241 | 0.010814765 | 216 | 11 | G11V | 0.383632995 | 0.017568947 |
| 176 | 70 | Y70S | 0.188310548 | 0.015329318 | 217 | 346 | Y346C | 0.387170111 | 0.01524906 |
| 177 | 346 | Y346S | 0.190074902 | 0.01880316 | 218 | 64 | L64P | 0.387670411 | 0.017607139 |
| 178 | 91 | I91S | 0.201032961 | 0.013430718 | 219 | 174 | V174G | 0.389703773 | 0.015269783 |
| 179 | 103 | V103G | 0.206543637 | 0.015581727 | 220 | 119 | G119V | 0.393854873 | 0.03057669 |
| 180 | 10 | F10S | 0.20866322 | 0.012094563 | 221 | 175 | C175F | 0.398035886 | 0.017704097 |
| 181 | 334 | G334W | 0.214506644 | 0.01921172 | 222 | 105 | G105E | 0.398082638 | 0.017704531 |
| 182 | 335 | H335L | 0.215599006 | 0.027191048 | 223 | 154 | N154 | 0.399043262 | 0.01086556 |
| 183 | 213 | R213G | 0.220196307 | 0.015763085 | 224 | 156 | T156S | 0.401749753 | 0.012555852 |
| 184 | 278 | I278S | 0.224805082 | 0.015822934 | 225 | 275 | S275P | 0.408073807 | 0.030802885 |
| 185 | 175 | C175R | 0.227952575 | 0.01229114 | 226 | 270 | L270P | 0.409424281 | 0.017808868 |
| 186 | 149 | V149E | 0.23029841 | 0.011243055 | 227 | 111 | A111V | 0.409640383 | 0.030827506 |
| 187 | 151 | Y151N | 0.230876574 | 0.011248283 | 228 | 340 | G340C | 0.411727801 | 0.021829242 |
| 188 | 89 | N89D | 0.240825557 | 0.016025928 | 229 | 181 | F181S | 0.417778394 | 0.017884596 |
| 189 | 282 | I282N | 0.253551504 | 0.01401625 | 230 | 247 | S247R | 0.419320759 | 0.021913041 |
| 190 | 302 | N302H | 0.253834708 | 0.014019218 | 231 | 335 | H335P | 0.429131492 | 0.031129066 |
| 191 | 291 | C291R | 0.257636655 | 0.016231069 | 232 | 155 | E155D | 0.438346608 | 0.009065438 |
| 192 | 199 | Q199E | 0.277049306 | 0.012753757 | 233 | 44 | H44Q | 0.453193052 | 0.015764441 |
| 193 | 179 | S179P | 0.277668503 | 0.010092833 | 234 | 191 | F191S | 0.456210755 | 0.031534144 |
| 194 | 340 | G340D | 0.277723948 | 0.01646656 | 235 | 303 | I303N | 0.456236737 | 0.018221708 |
| 195 | 307 | I307N | 0.279333417 | 0.020186058 | 236 | 104 | T104K | 0.457736662 | 0.022323216 |
| 196 | 115 | A115T | 0.294683232 | 0.020397449 | 237 | 119 | G119W | 0.461513749 | 0.011209729 |
| 197 | 91 | I91T | 0.294786295 | 0.014430187 | 238 | 276 | Q276P | 0.471699457 | 0.014228136 |
| 198 | 243 | P243H | 0.301863636 | 0.01673703 | 239 | 14 | P14A | 0.472185444 | 0.012997065 |
| 199 | 72 | V72E | 0.305608394 | 0.029047232 | 240 | 59 | L59P | 0.47733623 | 0.015941341 |
| 200 | 183 | S183P | 0.306386428 | 0.014540589 | 241 | 122 | N122I | 0.477509059 | 0.022525983 |
| 201 | 149 | V149G | 0.321080268 | 0.01467701 | 242 | 17 | L17R | 0.482388406 | 0.031911579 |
| 202 | 332 | L332S | 0.321549763 | 0.029341476 | 243 | 115 | A115V | 0.485305372 | 0.015998516 |
| 244 | 14 | P14S | 0.485798131 | 0.031959778 | 285 | 83 | F83Y | 0.564884664 | 0.033021808 |
| 245 | 11 | G11W | 0.48593221 | 0.018470136 | 286 | 86 | V86G | 0.567130265 | 0.02338401 |
| 246 | 299 | S299C | 0.486472656 | 0.022616169 | 287 | 335 | H335R | 0.572706012 | 0.033121499 |
| 247 | 22 | L22W | 0.487751499 | 0.018485044 | 288 | 99 | A99V | 0.573484384 | 0.002715355 |
| 248 | 364 | F364L | 0.487809309 | 0.022629527 | 289 | 292 | P292Q | 0.573946333 | 0.012569712 |
| 249 | 45 | R45W | 0.488258523 | 0.011348567 | 290 | 151 | Y151S | 0.574045196 | 0.016599052 |
| 250 | 241 | N241Y | 0.488607633 | 0.018492048 | 291 | 321 | L321P | 0.574171502 | 0.01485625 |
| 251 | 70 | Y70C | 0.491587496 | 0.018516365 | 292 | 213 | R213L | 0.576486177 | 0.033169365 |
| 252 | 75 | L75R | 0.492157677 | 0.014359815 | 293 | 211 | I211S | 0.57669275 | 0.023470306 |
| 253 | 201 | N201H | 0.492600859 | 0.022677226 | 294 | 89 | N89T | 0.581020561 | 0.013606092 |
| 254 | 334 | G334V | 0.494593531 | 0.032083122 | 295 | 161 | E161K | 0.584372326 | 0.019231314 |
| 255 | 121 | I121R | 0.496950644 | 0.022720273 | 296 | 320 | F320C | 0.589637969 | 0.033334326 |
| 256 | 87 | P87H | 0.497772096 | 0.022728376 | 297 | 180 | N180K | 0.591694722 | 0.013661912 |
| 257 | 230 | Y230D | 0.499269433 | 0.022743124 | 298 | 147 | S147T | 0.592100995 | 0.023607443 |
| 258 | 124 | V124D | 0.499569422 | 0.022746075 | 299 | 10 | F10I | 0.597684085 | 0.033434071 |
| 259 | 216 | L216R | 0.501239281 | 0.032175396 | 300 | 263 | G263V | 0.600518275 | 0.023681391 |
| 260 | 187 | D187Y | 0.503123591 | 0.032201416 | 301 | 292 | P292A | 0.602082139 | 0.023695056 |
| 261 | 87 | P87L | 0.514909465 | 0.018703571 | 302 | 245 | C245Y | 0.605150307 | 0.033525847 |
| 262 | 179 | S179A | 0.516164497 | 0.01621458 | 303 | 282 | I282S | 0.606074588 | 0.016801412 |
| 263 | 170 | G170V | 0.518491368 | 0.010296482 | 304 | 166 | P166L | 0.610121015 | 0.023764942 |
| 264 | 289 | Y289C | 0.518940982 | 0.016233621 | 305 | 364 | F364C | 0.611007361 | 0.019423077 |
| 265 | 358 | A358P | 0.519792956 | 0.022942506 | 306 | 288 | F288C | 0.611267526 | 0.016833582 |
| 266 | 293 | V293M | 0.51983056 | 0.022942866 | 307 | 340 | G340S | 0.612312728 | 0.033613197 |
| 267 | 178 | S178T | 0.520679036 | 0.010306184 | 308 | 166 | P166T | 0.612329202 | 0.033613397 |
| 268 | 287 | G287V | 0.526220036 | 0.023003914 | 309 | 282 | I282T | 0.618336039 | 0.023835748 |
| 269 | 67 | P67L | 0.529158341 | 0.018815336 | 310 | 292 | P292S | 0.619841138 | 0.019485497 |
| 270 | 205 | A20S | 0.529677048 | 0.014592813 | 311 | 303 | I303F | 0.620209034 | 0.015115782 |
| 271 | 140 | W140L | 0.534059222 | 0.032619977 | 312 | 161 | E161V | 0.621328368 | 0.016895419 |
| 272 | 154 | N154T | 0.540178962 | 0.016377214 | 313 | 278 | I278T | 0.621954424 | 0.023866742 |
| 273 | 292 | P292R | 0.540252974 | 0.032701895 | 314 | 111 | A111T | 0.622045907 | 0.01381732 |
| 274 | 293 | V293E | 0.54427183 | 0.032754725 | 315 | 197 | G197V | 0.625705579 | 0.023898751 |
| 275 | 339 | G339V | 0.54981991 | 0.023225391 | 316 | 153 | A153P | 0.626193688 | 0.010748249 |
| 276 | 275 | S275Y | 0.549866486 | 0.018974433 | 317 | 166 | P166H | 0.627686869 | 0.015156791 |
| 277 | 361 | M361T | 0.550401774 | 0.032834824 | 318 | 245 | C245F | 0.628300102 | 0.019544738 |
| 278 | 150 | Y150C | 0.551441251 | 0.018986375 | 319 | 213 | R213C | 0.630034074 | 0.012017426 |
| 279 | 60 | V60E | 0.551672341 | 0.016453453 | 320 | 64 | L64R | 0.633251333 | 0.016967889 |
| 280 | 343 | A343D | 0.55656662 | 0.023287587 | 321 | 361 | M361V | 0.63485932 | 0.013881524 |
| 281 | 140 | W140R | 0.558596862 | 0.023306209 | 322 | 335 | H335Q | 0.635801303 | 0.019596847 |
| 282 | 204 | S204Y | 0.559944585 | 0.023318547 | 323 | 104 | T104R | 0.636072704 | 0.016984911 |
| 283 | 364 | F364S | 0.56382133 | 0.023353931 | 324 | 335 | H335N | 0.63657811 | 0.016987955 |
| 284 | 193 | V193G | 0.564189298 | 0.019082267 | 325 | 344 | S344P | 0.636654343 | 0.023991472 |
| 326 | 285 | S285C | 0.639143078 | 0.017003381 | 367 | 139 | T139P | 0.683658515 | 0.015454466 |
| 327 | 166 | P166S | 0.64045269 | 0.019628962 | 368 | 172 | V172G | 0.687701499 | 0.024410639 |
| 328 | 216 | L216P | 0.641337862 | 0.033960492 | 369 | 163 | D163V | 0.688209741 | 0.017291226 |
| 329 | 320 | F320I | 0.644613789 | 0.033999037 | 370 | 112 | A112E | 0.688925695 | 0.017295328 |
| 330 | 140 | W140G | 0.647513162 | 0.019677425 | 371 | 285 | S285A | 0.690587935 | 0.024433727 |
| 331 | 338 | V338E | 0.651119042 | 0.024112399 | 372 | 324 | A324D | 0.694011025 | 0.015507835 |
| 332 | 206 | G206V | 0.653275821 | 0.01971673 | 373 | 366 | E366V | 0.694228669 | 0.034567685 |
| 333 | 66 | V66F | 0.653736025 | 0.019719859 | 374 | 282 | 1282F | 0.695224851 | 0.034578823 |
| 334 | 228 | L228P | 0.654724848 | 0.024142272 | 375 | 158 | H158Y | 0.695450359 | 0.024472478 |
| 335 | 364 | F364V | 0.65537621 | 0.024147656 | 376 | 181 | F181C | 0.696083062 | 0.034588411 |
| 336 | 195 | F195S | 0.655457024 | 0.034125712 | 377 | 279 | Y279D | 0.697792852 | 0.024491084 |
| 337 | 43 | S43R | 0.657871438 | 0.017114848 | 378 | 359 | A359P | 0.700390061 | 0.034636409 |
| 338 | 233 | Q233P | 0.658716845 | 0.019753637 | 379 | 191 | F191V | 0.701501819 | 0.034648766 |
| 339 | 140 | W140C | 0.659814215 | 0.017126295 | 380 | 347 | N347Y | 0.702893301 | 0.034664215 |
| 340 | 279 | Y279H | 0.660088477 | 0.024186511 | 381 | 281 | I281F | 0.703510279 | 0.024536325 |
| 341 | 188 | V188G | 0.661508965 | 0.019772501 | 382 | 275 | S275T | 0.70405624 | 0.024540633 |
| 342 | 61 | R61W | 0.662208385 | 0.017140373 | 383 | 274 | K274I | 0.704291778 | 0.020055359 |
| 343 | 298 | R298I | 0.663768155 | 0.024216725 | 384 | 12 | P12T | 0.707721377 | 0.024569494 |
| 344 | 146 | A146D | 0.664352516 | 0.019791659 | 385 | 279 | Y279F | 0.708549836 | 0.017406732 |
| 345 | 283 | D283H | 0.665028788 | 0.019796208 | 386 | 303 | I303M | 0.710019895 | 0.024587544 |
| 346 | 63 | L63W | 0.665623043 | 0.03424323 | 387 | 92 | G92V | 0.710279528 | 0.02458958 |
| 347 | 303 | I303S | 0.66573582 | 0.034244527 | 388 | 292 | P292L | 0.711639178 | 0.01742409 |
| 348 | 42 | M42R | 0.666115487 | 0.017163278 | 389 | 166 | P166A | 0.713288276 | 0.017433337 |
| 349 | 224 | C224Y | 0.666948377 | 0.017168149 | 390 | 213 | R213S | 0.713744947 | 0.024616715 |
| 350 | 285 | S285T | 0.667525293 | 0.024247464 | 391 | 293 | V293A | 0.714644013 | 0.020122132 |
| 351 | 209 | V209E | 0.667793207 | 0.024249652 | 392 | 177 | M177K | 0.716338593 | 0.034812459 |
| 352 | 320 | F320L | 0.670015455 | 0.024267776 | 393 | 186 | V186G | 0.717175546 | 0.034821625 |
| 353 | 364 | F364I | 0.671593524 | 0.017195249 | 394 | 298 | R298K | 0.718547914 | 0.034836641 |
| 354 | 174 | V174L | 0.672152189 | 0.034318084 | 395 | 163 | D163Y | 0.71942511 | 0.024661006 |
| 355 | 357 | L357Q | 0.673355158 | 0.019851971 | 396 | 328 | N328I | 0.721745514 | 0.020167578 |
| 356 | 298 | R298S | 0.676302004 | 0.024318835 | 397 | 134 | I134L | 0.721799956 | 0.024679456 |
| 357 | 307 | I307S | 0.676365896 | 0.024319352 | 398 | 210 | V210E | 0.722043002 | 0.020169475 |
| 358 | 103 | V103E | 0.676820083 | 0.034371306 | 399 | 60 | V60G | 0.722799381 | 0.017486402 |
| 359 | 175 | C175Y | 0.677872048 | 0.019882035 | 400 | 298 | R298G | 0.723175528 | 0.020176694 |
| 360 | 317 | E317D | 0.678727807 | 0.024338455 | 401 | 202 | V202G | 0.724570716 | 0.020185578 |
| 361 | 298 | R298T | 0.680514243 | 0.019899563 | 402 | 279 | Y2795 | 0.725502461 | 0.020191504 |
| 362 | 106 | A106T | 0.681342877 | 0.01990505 | 403 | 353 | D353A | 0.727612659 | 0.024724448 |
| 363 | 341 | I341N | 0.681639773 | 0.013072893 | 404 | 206 | G206E | 0.728913673 | 0.020213159 |
| 364 | 216 | L216Q | 0.683314206 | 0.024375425 | 405 | 21 | V21E | 0.729839703 | 0.024741624 |
| 365 | 180 | N180Y | 0.683564449 | 0.024377437 | 406 | 134 | I134T | 0.730291701 | 0.024745106 |
| 366 | 317 | E317G | 0.683626195 | 0.014118821 | 407 | 89 | N89S | 0.730322357 | 0.034964695 |
| 408 | 264 | A264D | 0.73071651 | 0.024748377 | 449 | 104 | T104 | 0.766652709 | 0.025020696 |
| 409 | 80 | C80R | 0.731988901 | 0.034982709 | 450 | 15 | A15P | 0.768745672 | 0.017736693 |
| 410 | 277 | T277P | 0.733092835 | 0.024766652 | 451 | 288 | F288V | 0.771137312 | 0.025054088 |
| 411 | 100 | D100Y | 0.735217632 | 0.024782959 | 452 | 33 | K33I | 0.772116394 | 0.035408451 |
| 412 | 49 | F49S | 0.735416883 | 0.024784487 | 453 | 257 | W257S | 0.77455237 | 0.035433817 |
| 413 | 233 | Q233R | 0.735808984 | 0.035023898 | 454 | 274 | K274E | 0.775155271 | 0.015909845 |
| 414 | 320 | F320Y | 0.738694541 | 0.024809578 | 455 | 267 | M267K | 0.775709393 | 0.020503845 |
| 415 | 162 | F162V | 0.738913559 | 0.024811252 | 456 | 302 | N302D | 0.776140826 | 0.035450329 |
| 416 | 197 | G197S | 0.739967887 | 0.035068578 | 457 | 281 | I281N | 0.776776909 | 0.020510342 |
| 417 | 262 | G262V | 0.7403841 | 0.024822484 | 458 | 191 | F191L | 0.777130727 | 0.020512495 |
| 418 | 230 | Y230S | 0.740967168 | 0.01332954 | 459 | 79 | G79A | 0.777873431 | 0.009950077 |
| 419 | 134 | I134N | 0.74098913 | 0.0248271 | 460 | 347 | N347T | 0.778694416 | 0.010349282 |
| 420 | 15 | A15D | 0.742038921 | 0.020295877 | 461 | 100 | D100G | 0.778992046 | 0.015928187 |
| 421 | 94 | K94I | 0.742741262 | 0.024840456 | 462 | 211 | I211T | 0.779526923 | 0.035485453 |
| 422 | 368 | H368R | 0.744358039 | 0.020310394 | 463 | 305 | F305L | 0.780485038 | 0.015935309 |
| 423 | 70 | Y70H | 0.744879749 | 0.035121134 | 464 | 83 | F83C | 0.780569843 | 0.025123908 |
| 424 | 134 | I134S | 0.745871845 | 0.014406983 | 465 | 366 | E366K | 0.780850063 | 0.03549915 |
| 425 | 307 | I307T | 0.746114802 | 0.024866113 | 466 | 303 | I303T | 0.781106747 | 0.017802397 |
| 426 | 353 | D353Y | 0.746121063 | 0.024866161 | 467 | 182 | L182P | 0.781483948 | 0.020538923 |
| 427 | 358 | A358D | 0.746534315 | 0.024869299 | 468 | 291 | C291S | 0.783137584 | 0.020548937 |
| 428 | 114 | E114K | 0.748864882 | 0.035163605 | 469 | 186 | V186E | 0.786059574 | 0.025164288 |
| 429 | 290 | V290G | 0.749187027 | 0.024889413 | 470 | 293 | V293L | 0.786532551 | 0.014587281 |
| 430 | 160 | V160M | 0.750034045 | 0.020345802 | 471 | 329 | M329V | 0.786689174 | 0.020570399 |
| 431 | 160 | V160G | 0.751384499 | 0.02490604 | 472 | 316 | L316S | 0.787204234 | 0.015967255 |
| 432 | 118 | F118S | 0.752079224 | 0.02491129 | 473 | 281 | 1281S | 0.787444057 | 0.03556718 |
| 433 | 286 | Q286P | 0.752727059 | 0.020362541 | 474 | 162 | F162S | 0.787852194 | 0.020577413 |
| 434 | 204 | S204P | 0.753725299 | 0.020368736 | 475 | 163 | D163H | 0.788147691 | 0.035574417 |
| 435 | 124 | V124G | 0.755844904 | 0.035237632 | 476 | 100 | D100V | 0.788279645 | 0.025180566 |
| 436 | 56 | T56R | 0.756071292 | 0.024941397 | 477 | 288 | F288I | 0.790662586 | 0.025198004 |
| 437 | 158 | H158R | 0.756077185 | 0.00953039 | 478 | 267 | M267R | 0.790839762 | 0.025199299 |
| 438 | 329 | M329I | 0.759132557 | 0.020402202 | 479 | 348 | A348G | 0.791700193 | 0.017858182 |
| 439 | 347 | N347D | 0.761200639 | 0.015842653 | 480 | 283 | D283V | 0.791874806 | 0.025206862 |
| 440 | 161 | E161D | 0.761574246 | 0.017698265 | 481 | 99 | A99G | 0.793486955 | 0.014617539 |
| 441 | 288 | F288S | 0.762405262 | 0.024988952 | 482 | 204 | S204F | 0.794767661 | 0.025227964 |
| 442 | 162 | F162I | 0.762597089 | 0.035308812 | 483 | 67 | P67R | 0.79586268 | 0.025235938 |
| 443 | 248 | I248S | 0.762644786 | 0.035309313 | 484 | 5 | R5G | 0.79597438 | 0.010006425 |
| 444 | 333 | K333N | 0.763280272 | 0.024995501 | 485 | 225 | P225L | 0.79612402 | 0.017881337 |
| 445 | 180 | N180H | 0.763633795 | 0.012568876 | 486 | 230 | Y230H | 0.79613856 | 0.035656302 |
| 446 | 182 | L182Q | 0.764029347 | 0.025001103 | 487 | 17 | L17M | 0.797880745 | 0.025250616 |
| 447 | 61 | R61I | 0.76475665 | 0.017715346 | 488 | 124 | V124F | 0.798645343 | 0.02525617 |
| 448 | 10 | F10V | 0.766130532 | 0.017722706 | 489 | 162 | F162C | 0.799713632 | 0.025263926 |
| 490 | 102 | V102L | 0.800055252 | 0.035696238 | 531 | 83 | F83V | 0.82075613 | 0.016124278 |
| 491 | 134 | I134F | 0.800804023 | 0.025271834 | 532 | 49 | F49C | 0.821164901 | 0.013657347 |
| 492 | 278 | I278F | 0.800819945 | 0.035704019 | 533 | 187 | D187G | 0.82176799 | 0.018013976 |
| 493 | 77 | G77E | 0.801632029 | 0.017910053 | 534 | 283 | 283G | 0.821840633 | 0.018014348 |
| 494 | 21 | V21G | 0.802047875 | 0.017912216 | 535 | 316 | 316I | 0.822014333 | 0.035917749 |
| 495 | 160 | V160A | 0.802052887 | 0.025280883 | 536 | 4 | P4L | 0.82201664 | 0.025424298 |
| 496 | 209 | V209G | 0.802630833 | 0.016039961 | 537 | 100 | D100A | 0.822203307 | 0.003127135 |
| 497 | 288 | F288L | 0.803101783 | 0.017917694 | 538 | 190 | K190N | 0.822222655 | 0.035919831 |
| 498 | 360 | F360S | 0.80372736 | 0.025293002 | 539 | 15 | A15V | 0.822394417 | 0.025426989 |
| 499 | 224 | C224R | 0.803798424 | 0.025293516 | 540 | 356 | K356R | 0.822477821 | 0.035922381 |
| 500 | 181 | F181L | 0.804116855 | 0.025295818 | 541 | 220 | A220D | 0.823962181 | 0.020791948 |
| 501 | 230 | Y230C | 0.805466148 | 0.020682837 | 542 | 280 | E280K | 0.824906862 | 0.025444869 |
| 502 | 89 | N89H | 0.805484507 | 0.020682947 | 543 | 108 | S108T | 0.825055283 | 0.016144105 |
| 503 | 189 | S189F | 0.805828224 | 0.035754862 | 544 | 32 | Y32D | 0.825133435 | 0.03594889 |
| 504 | 282 | I282M | 0.805892101 | 0.017932177 | 545 | 263 | G263R | 0.825348125 | 0.02080006 |
| 505 | 191 | F191C | 0.806900784 | 0.016059931 | 546 | 354 | V354G | 0.825491265 | 0.012804608 |
| 506 | 314 | D314A | 0.807452328 | 0.025319901 | 547 | 84 | S84T | 0.825604328 | 0.018033591 |
| 507 | 75 | L75V | 0.80749895 | 0.035771776 | 548 | 361 | M361L | 0.825703331 | 0.020802137 |
| 508 | 10 | F10C | 0.808408898 | 0.025326795 | 549 | 307 | I307F | 0.826347574 | 0.020805904 |
| 509 | 178 | S178C | 0.808431782 | 0.02532696 | 550 | 250 | V250F | 0.826691798 | 0.035964419 |
| 510 | 20 | S20L | 0.808877203 | 0.025330169 | 551 | 225 | P225H | 0.826909852 | 0.025459099 |
| 511 | 56 | T56P | 0.809996511 | 0.017953422 | 552 | 74 | F74S | 0.82971074 | 0.016165504 |
| 512 | 67 | P67A | 0.810026707 | 0.013613016 | 553 | 268 | E268V | 0.830124276 | 0.035998555 |
| 513 | 306 | R306G | 0.811462207 | 0.020718385 | 554 | 114 | E114A | 0.831077618 | 0.020833501 |
| 514 | 69 | N69I | 0.812554401 | 0.013623109 | 555 | 366 | E366A | 0.83170405 | 0.036014234 |
| 515 | 258 | I258T | 0.813079592 | 0.020727945 | 556 | 299 | S299T | 0.832264169 | 0.025497024 |
| 516 | 134 | I134M | 0.813906571 | 0.0136285 | 557 | 284 | N284I | 0.832672161 | 0.036023832 |
| 517 | 190 | K190E | 0.814285997 | 0.020735067 | 558 | 234 | A234P | 0.832803159 | 0.036025131 |
| 518 | 57 | E57K | 0.814292372 | 0.03584031 | 559 | 101 | Y101H | 0.833586252 | 0.020848096 |
| 519 | 179 | S179T | 0.814598844 | 0.014708407 | 560 | 353 | D353G | 0.833852499 | 0.025508243 |
| 520 | 126 | P126A | 0.815044723 | 0.035847877 | 561 | 168 | V168D | 0.834229233 | 0.013708883 |
| 521 | 305 | F305Y | 0.815313898 | 0.035850583 | 562 | 218 | G218W | 0.834365389 | 0.025511863 |
| 522 | 158 | H158L | 0.815386384 | 0.014711768 | 563 | 67 | P67T | 0.834429717 | 0.025512317 |
| 523 | 335 | H335Y | 0.81737908 | 0.035871324 | 564 | 106 | A106P | 0.83473823 | 0.036044292 |
| 524 | 341 | 341L | 0.817599076 | 0.017992594 | 565 | 152 | C152G | 0.834830188 | 0.010527461 |
| 525 | 305 | F305V | 0.81883355 | 0.03588591 | 566 | 18 | P18Q | 0.835664278 | 0.025521024 |
| 526 | 317 | E317K | 0.819001229 | 0.025402784 | 567 | 194 | I194S | 0.835844478 | 0.025522294 |
| 527 | 277 | T277R | 0.819226427 | 0.035889847 | 568 | 98 | C98F | 0.836096153 | 0.02086267 |
| 528 | 83 | F83I | 0.819351144 | 0.016117784 | 569 | 68 | D68Y | 0.836307973 | 0.018088014 |
| 529 | 87 | P87S | 0.819536661 | 0.01800254 | 570 | 99 | A99S | 0.836879259 | 0.020867211 |
| 530 | 244 | P244L | 0.820181069 | 0.020769773 | 571 | 90 | L90F | 0.837293879 | 0.025532504 |
| 572 | 202 | V202A | 0.83743934 | 0.025533528 | 613 | 346 | Y346H | 0.854270189 | 0.018178358 |
| 573 | 54 | N54I | 0.837779805 | 0.018095463 | 614 | 352 | E352V | 0.854328575 | 0.036236619 |
| 574 | 103 | V103M | 0.837866104 | 0.0180959 | 615 | 229 | E229D | 0.854517326 | 0.036238458 |
| 575 | 290 | V290L | 0.837995367 | 0.025537441 | 616 | 181 | F181V | 0.854704042 | 0.018180525 |
| 576 | 67 | P67Q | 0.83890535 | 0.016207547 | 617 | 86 | V86F | 0.854756167 | 0.036240784 |
| 577 | 151 | Y151C | 0.838948099 | 0.01810137 | 618 | 197 | G197A | 0.855111376 | 0.013790258 |
| 578 | 49 | F49L | 0.839424329 | 0.02554749 | 619 | 187 | D187V | 0.855145822 | 0.020972371 |
| 579 | 58 | N58K | 0.840001402 | 0.036096258 | 620 | 106 | A106G | 0.855354532 | 0.036246609 |
| 580 | 230 | Y230N | 0.840559875 | 0.025555467 | 621 | 54 | N54T | 0.855443489 | 0.025659377 |
| 581 | 327 | L327P | 0.840881425 | 0.020890378 | 622 | 162 | F162L | 0.855943441 | 0.025662847 |
| 582 | 338 | V338G | 0.841236827 | 0.016218162 | 623 | 181 | F181I | 0.856586264 | 0.018189918 |
| 583 | 203 | G203C | 0.841510825 | 0.018114309 | 624 | 333 | K333Q | 0.857006633 | 0.020983001 |
| 584 | 102 | V102A | 0.84153231 | 0.025562293 | 625 | 136 | D136A | 0.857215028 | 0.036264706 |
| 585 | 242 | T242A | 0.841726018 | 0.025563652 | 626 | 67 | P67S | 0.857305603 | 0.020984708 |
| 586 | 61 | R61Q | 0.841882442 | 0.020896161 | 627 | 136 | D136G | 0.85762089 | 0.020986507 |
| 587 | 49 | F49V | 0.842263183 | 0.020898359 | 628 | 339 | G339E | 0.857726085 | 0.036269672 |
| 588 | 354 | V354A | 0.842901252 | 0.036124796 | 629 | 68 | D68A | 0.857932525 | 0.020988285 |
| 589 | 135 | P135L | 0.843049491 | 0.018122065 | 630 | 257 | W257C | 0.858064613 | 0.03627296 |
| 590 | 190 | K190T | 0.843211111 | 0.02090383 | 631 | 262 | G262A | 0.858409833 | 0.018199007 |
| 591 | 194 | I194F | 0.84340222 | 0.018123842 | 632 | 274 | K274R | 0.858868594 | 0.025683121 |
| 592 | 109 | A109V | 0.843771237 | 0.020907061 | 633 | 84 | S84I | 0.859477733 | 0.018204323 |
| 593 | 125 | H125D | 0.845088399 | 0.025587211 | 634 | 248 | I248N | 0.859609695 | 0.036287959 |
| 594 | 163 | D163A | 0.846197988 | 0.025594971 | 635 | 159 | G159C | 0.86016135 | 0.025692066 |
| 595 | 46 | S46P | 0.846469305 | 0.018139272 | 636 | 71 | K71N | 0.860383624 | 0.021002256 |
| 596 | 101 | Y101F | 0.846784659 | 0.025599072 | 637 | 42 | M42V | 0.860972321 | 0.025697673 |
| 597 | 337 | S337P | 0.847931855 | 0.025607085 | 638 | 349 | V349D | 0.861224107 | 0.018213009 |
| 598 | 51 | K51Q | 0.847948461 | 0.018146701 | 639 | 282 | I282L | 0.861680078 | 0.036308028 |
| 599 | 59 | L59V | 0.84850922 | 0.020934334 | 640 | 328 | N328Y | 0.862792167 | 0.018220798 |
| 600 | 297 | N297T | 0.848653845 | 0.01815024 | 641 | 228 | L228R | 0.863355768 | 0.036324247 |
| 601 | 124 | V124A | 0.848758627 | 0.020935767 | 642 | 76 | Q76E | 0.864276325 | 0.02102439 |
| 602 | 307 | I307M | 0.850379557 | 0.025624158 | 643 | 212 | V212G | 0.864866899 | 0.036338856 |
| 603 | 266 | A266P | 0.850416021 | 0.020945282 | 644 | 84 | S84C | 0.864883857 | 0.018231174 |
| 604 | 280 | E280V | 0.850640065 | 0.025625973 | 645 | 222 | R222G | 0.865224023 | 0.021029769 |
| 605 | 159 | G159A | 0.850732213 | 0.012173361 | 646 | 326 | E326Q | 0.865359108 | 0.025727943 |
| 606 | 224 | C224F | 0.850883494 | 0.025627669 | 647 | 35 | V35F | 0.866049574 | 0.021034451 |
| 607 | 149 | V149A | 0.851154152 | 0.018162772 | 648 | 43 | S43I | 0.869210273 | 0.018252585 |
| 608 | 116 | K116R | 0.851649255 | 0.036210491 | 649 | 7 | V7G | 0.869448748 | 0.025756072 |
| 609 | 154 | N154H | 0.852606858 | 0.018170043 | 650 | 183 | S183T | 0.86978644 | 0.025758391 |
| 610 | 263 | G263D | 0.852848615 | 0.025641347 | 651 | 76 | Q76H | 0.870376756 | 0.012969158 |
| 611 | 52 | I52M | 0.853085977 | 0.020960585 | 652 | 190 | K190R | 0.871132248 | 0.018262075 |
| 612 | 240 | Y240D | 0.854066425 | 0.025649814 | 653 | 66 | V66L | 0.871174078 | 0.021063453 |
| 654 | 130 | S130R | 0.871256814 | 0.02576848 | 695 | 258 | I258S | 0.883130519 | 0.018321018 |
| 655 | 276 | Q2768 | 0.871902963 | 0.021067569 | 696 | 161 | E161G | 0.883255475 | 0.036515265 |
| 656 | 305 | F305I | 0.873227938 | 0.025781988 | 697 | 137 | P137R | 0.883516589 | 0.036517752 |
| 657 | 40 | L40I | 0.873767025 | 0.025785679 | 698 | 284 | N284Y | 0.883720687 | 0.018323905 |
| 658 | 287 | 3287F | 0.87386167 | 0.025786326 | 699 | 216 | L216M | 0.884621419 | 0.018328307 |
| 659 | 104 | T104S | 0.874712927 | 0.025792151 | 700 | 187 | D187A | 0.886370571 | 0.036544904 |
| 660 | 343 | A343G | 0.874941354 | 0.018280843 | 701 | 343 | A343S | 0.887306319 | 0.036553793 |
| 661 | 167 | D167V | 0.875040102 | 0.016370043 | 702 | 279 | Y279N | 0.887586775 | 0.036556456 |
| 662 | 235 | G235V | 0.875180064 | 0.036438101 | 703 | 51 | K51R | 0.88765417 | 0.036557096 |
| 663 | 220 | A220V | 0.875675721 | 0.025798734 | 704 | 123 | I123F | 0.887663864 | 0.021156032 |
| 664 | 254 | V254D | 0.875961387 | 0.021090446 | 705 | 21 | V21A | 0.888110944 | 0.025883335 |
| 665 | 77 | G77V | 0.875968326 | 0.021090486 | 706 | 130 | S130T | 0.888184587 | 0.021158937 |
| 666 | 157 | V157G | 0.876252523 | 0.018287292 | 707 | 36 | G36C | 0.888522273 | 0.016429597 |
| 667 | 213 | R213H | 0.876345749 | 0.01828775 | 708 | 308 | G308D | 0.888813046 | 0.025888088 |
| 668 | 239 | 239V | 0.876609635 | 0.036451796 | 709 | 188 | V188F | 0.888939528 | 0.036569293 |
| 669 | 238 | S238P | 0.876697446 | 0.021094588 | 710 | 126 | P126R | 0.889451772 | 0.021166003 |
| 670 | 187 | D187H | 0.876940209 | 0.018290671 | 711 | 215 | D215V | 0.889875041 | 0.025895273 |
| 671 | 302 | N302S | 0.876958243 | 0.01829076 | 712 | 33 | K33R | 0.889905984 | 0.021168534 |
| 672 | 275 | S275C | 0.877095086 | 0.021096825 | 713 | 207 | V207F | 0.890491818 | 0.025899443 |
| 673 | 339 | G339A | 0.877540758 | 0.025811473 | 714 | 100 | D100H | 0.891500878 | 0.036593564 |
| 674 | 217 | L217M | 0.877830694 | 0.036463481 | 715 | 183 | S183Y | 0.892235238 | 0.025911221 |
| 675 | 196 | A196T | 0.878350463 | 0.025816998 | 716 | 328 | N328S | 0.892437407 | 0.021182624 |
| 676 | 220 | A220P | 0.878647527 | 0.036471292 | 717 | 91 | I91V | 0.892984935 | 0.016449186 |
| 677 | 208 | T208I | 0.879165554 | 0.02110846 | 718 | 127 | K127R | 0.893350295 | 0.021187699 |
| 678 | 100 | D100E | 0.879210014 | 0.02110871 | 719 | 217 | L217R | 0.893509184 | 0.025919817 |
| 679 | 247 | S247C | 0.87951351 | 0.021110414 | 720 | 91 | I91F | 0.893516921 | 0.01393685 |
| 680 | 264 | A264G | 0.879627645 | 0.036480658 | 721 | 143 | N143K | 0.893614779 | 0.018372109 |
| 681 | 62 | E62V | 0.879649854 | 0.03648087 | 722 | 201 | N201K | 0.89367861 | 0.02592096 |
| 682 | 161 | E161Q | 0.880407865 | 0.025831021 | 723 | 21 | V21L | 0.893832481 | 0.018373166 |
| 683 | 290 | V290D | 0.880502939 | 0.025831669 | 724 | 297 | N297D | 0.893867114 | 0.018373334 |
| 684 | 183 | S183C | 0.880580226 | 0.018308532 | 725 | 262 | G262R | 0.89397639 | 0.025922968 |
| 685 | 6 | Q6H | 0.880863164 | 0.036492455 | 726 | 312 | G312V | 0.894072677 | 0.025923617 |
| 686 | 167 | D167Y | 0.880999784 | 0.025835052 | 727 | 135 | P135T | 0.894466403 | 0.036621606 |
| 687 | 19 | H19L | 0.881108501 | 0.021119363 | 728 | 69 | N69D | 0.894836384 | 0.013941818 |
| 688 | 165 | I165L | 0.881183844 | 0.01831149 | 729 | 55 | N55I | 0.894916902 | 0.0211964 |
| 689 | 299 | S299G | 0.881229389 | 0.018311713 | 730 | 60 | V60A | 0.895282059 | 0.018380199 |
| 690 | 20 | S20P | 0.88172317 | 0.025839975 | 731 | 69 | N69Y | 0.895482182 | 0.036631197 |
| 691 | 32 | Y32F | 0.881922264 | 0.021123924 | 732 | 250 | V250D | 0.89554547 | 0.036631794 |
| 692 | 98 | C98W | 0.882537096 | 0.036508419 | 733 | 165 | I165R | 0.895696873 | 0.036633223 |
| 693 | 175 | C175S | 0.882711692 | 0.025846699 | 734 | 300 | K300T | 0.895737893 | 0.03663361 |
| 694 | 153 | A153T | 0.882876763 | 0.021129271 | 735 | 201 | N20I | 0.896044029 | 0.018383893 |
| 736 | 323 | K323N | 0.896334237 | 0.016463847 | 777 | 145 | D145V | 0.904196863 | 0.036713179 |
| 737 | 53 | I53N | 0.896614501 | 0.025940738 | 778 | 24 | E24D | 0.904889595 | 0.025996254 |
| 738 | 20 | S20T | 0.896667072 | 0.025941091 | 779 | 112 | A112T | 0.904947781 | 0.021251882 |
| 739 | 276 | Q276L | 0.896914936 | 0.025942759 | 780 | 323 | K323R | 0.905274348 | 0.036723278 |
| 740 | 190 | K190Q | 0.897204572 | 0.018389515 | 781 | 296 | Q296P | 0.905451542 | 0.026000012 |
| 741 | 128 | L128V | 0.897466753 | 0.036649913 | 782 | 43 | 543G | 0.905869765 | 0.036728856 |
| 742 | 204 | S204C | 0.897469553 | 0.018390798 | 783 | 351 | I351V | 0.905898301 | 0.021257118 |
| 743 | 187 | D187N | 0.897719602 | 0.036652296 | 784 | 69 | N69K | 0.906130689 | 0.018432604 |
| 744 | 83 | F83L | 0.897840028 | 0.016470427 | 785 | 334 | G334R | 0.906164748 | 0.021258586 |
| 745 | 215 | D215N | 0.898113957 | 0.036656011 | 786 | 222 | R222P | 0.906476953 | 0.021260305 |
| 746 | 353 | D353N | 0.898251117 | 0.036657303 | 787 | 120 | T120S | 0.906647547 | 0.021261244 |
| 747 | 19 | H19Q | 0.898391863 | 0.021215666 | 788 | 28 | E28A | 0.907207327 | 0.026011743 |
| 748 | 119 | G119A | 0.898459909 | 0.021216043 | 789 | 366 | E366G | 0.907380923 | 0.018438618 |
| 749 | 57 | E57D | 0.898703355 | 0.025954783 | 790 | 168 | V168I | 0.907712289 | 0.036746099 |
| 750 | 334 | G334E | 0.898731966 | 0.016474322 | 791 | 72 | V72A | 0.907758218 | 0.026015421 |
| 751 | 265 | A265T | 0.898838036 | 0.01839742 | 792 | 353 | D353H | 0.90792929 | 0.021268296 |
| 752 | 32 | Y32H | 0.89916833 | 0.025957907 | 793 | 341 | I341S | 0.907954809 | 0.036748367 |
| 753 | 73 | I73S | 0.89957661 | 0.025960649 | 794 | 93 | L93S | 0.907981141 | 0.018441503 |
| 754 | 108 | S108A | 0.899759621 | 0.018401877 | 795 | 142 | L142H | 0.908048482 | 0.026017358 |
| 755 | 35 | V35I | 0.899801436 | 0.036671896 | 796 | 139 | T139N | 0.90854292 | 0.016517003 |
| 756 | 81 | G81S | 0.900004337 | 0.018403059 | 797 | 31 | D31E | 0.908592941 | 0.026020991 |
| 757 | 186 | V186M | 0.900061839 | 0.018403337 | 798 | 137 | P137T | 0.908752087 | 0.026022052 |
| 758 | 165 | I165K | 0.900395963 | 0.025966149 | 799 | 181 | F181Y | 0.90884184 | 0.021273313 |
| 759 | 331 | S331T | 0.901205202 | 0.021231228 | 800 | 309 | N309D | 0.908957355 | 0.021273947 |
| 760 | 23 | L23I | 0.901208812 | 0.025971602 | 801 | 103 | V103A | 0.90899839 | 0.036758122 |
| 761 | 317 | E317Q | 0.901586175 | 0.025974132 | 802 | 75 | L75P | 0.909117597 | 0.01237244 |
| 762 | 39 | V39L | 0.901644705 | 0.025974525 | 803 | 85 | A85V | 0.909176077 | 0.018447243 |
| 763 | 139 | T139S | 0.901666919 | 0.036689434 | 804 | 125 | H125P | 0.909224233 | 0.036760232 |
| 764 | 126 | P126L | 0.901954446 | 0.036692135 | 805 | 222 | R222L | 0.909226007 | 0.036760248 |
| 765 | 19 | H19D | 0.902048297 | 0.018412931 | 806 | 163 | D163E | 0.909250181 | 0.018447599 |
| 766 | 18 | P18A | 0.902533695 | 0.016490895 | 807 | 215 | D215H | 0.909272213 | 0.018447705 |
| 767 | 212 | V212F | 0.902643086 | 0.025981216 | 808 | 333 | K333E | 0.909374385 | 0.036761634 |
| 768 | 81 | G81C | 0.903052312 | 0.013085817 | 809 | 129 | G129W | 0.909510013 | 0.021276984 |
| 769 | 158 | H158Q | 0.90310801 | 0.013086013 | 810 | 309 | N309I | 0.909525958 | 0.03676305 |
| 770 | 173 | L173P | 0.903479 | 0.018419832 | 811 | 369 | Q369L | 0.909666799 | 0.036764365 |
| 771 | 348 | A348V | 0.903888839 | 0.03671029 | 812 | 170 | G170A | 0.909761077 | 0.021278363 |
| 772 | 316 | L316F | 0.903931195 | 0.025989842 | 813 | 306 | R306C | 0.909822656 | 0.018450347 |
| 773 | 180 | N180I | 0.904004239 | 0.015077982 | 814 | 247 | S247I | 0.910030135 | 0.036767758 |
| 774 | 57 | E57V | 0.904009427 | 0.025990365 | 815 | 103 | V103L | 0.910147763 | 0.018451908 |
| 775 | 235 | G235E | 0.90404095 | 0.01842254 | 816 | 18 | P18R | 0.910869596 | 0.021284449 |
| 776 | 304 | P304A | 0.904149893 | 0.036712739 | 817 | 347 | N347H | 0.910908598 | 0.026036425 |
| 818 | 57 | E57G | 0.911154182 | 0.018456735 | 859 | 188 | V188A | 0.918863143 | 0.026089244 |
| 819 | 25 | I25M | 0.911339897 | 0.021287029 | 860 | 369 | Q369R | 0.919135294 | 0.026091046 |
| 820 | 244 | P244S | 0.91148105 | 0.026040236 | 861 | 125 | H125L | 0.91919313 | 0.036853017 |
| 821 | 257 | W257G | 0.911496613 | 0.021287889 | 862 | 256 | E256Q | 0.919378832 | 0.036854739 |
| 822 | 325 | L325I | 0.911534621 | 0.026040593 | 863 | 56 | T56A | 0.919486616 | 0.018496576 |
| 823 | 273 | I273S | 0.911807008 | 0.026042405 | 864 | 268 | E268K | 0.92000931 | 0.02609683 |
| 824 | 123 | I123V | 0.911912593 | 0.015109575 | 865 | 137 | P137Q | 0.920337847 | 0.021336236 |
| 825 | 300 | K300E | 0.911921021 | 0.036785399 | 866 | 54 | N54D | 0.920771965 | 0.03686765 |
| 826 | 53 | I53S | 0.911960508 | 0.026043427 | 867 | 59 | L59I | 0.92089017 | 0.013148456 |
| 827 | 82 | Q82L | 0.912252812 | 0.026045372 | 868 | 57 | E57A | 0.920950548 | 0.018503552 |
| 828 | 327 | L327H | 0.912490257 | 0.026046951 | 869 | 147 | S147A | 0.921052988 | 0.021340133 |
| 829 | 270 | L270R | 0.912744126 | 0.021294729 | 870 | 94 | K94E | 0.921137179 | 0.021340592 |
| 830 | 36 | G36V | 0.912770202 | 0.021294872 | 871 | 51 | K51M | 0.921149265 | 0.021340658 |
| 831 | 266 | A266V | 0.9131976 | 0.021297214 | 872 | 237 | S237N | 0.921297567 | 0.036872517 |
| 832 | 21 | V21M | 0.913295265 | 0.013121873 | 873 | 60 | V60L | 0.921312159 | 0.021341545 |
| 833 | 282 | I282V | 0.913568042 | 0.021299243 | 874 | 253 | L253M | 0.921610239 | 0.036875412 |
| 834 | 300 | K300M | 0.914228506 | 0.036806894 | 875 | 236 | N236H | 0.921731961 | 0.021343832 |
| 835 | 271 | S271G | 0.914262879 | 0.026058735 | 876 | 235 | G235A | 0.921761278 | 0.018507413 |
| 836 | 189 | S189P | 0.914328521 | 0.036807825 | 877 | 211 | I211F | 0.9217854 | 0.036877034 |
| 837 | 50 | A50G | 0.914416969 | 0.021303892 | 878 | 16 | K16Q | 0.921870589 | 0.021344587 |
| 838 | 224 | C224S | 0.914429523 | 0.021303961 | 879 | 65 | A65V | 0.922063631 | 0.026110411 |
| 839 | 124 | V124L | 0.914608751 | 0.016543248 | 880 | 283 | D283A | 0.922201438 | 0.036880884 |
| 840 | 52 | I52L | 0.914699022 | 0.021305436 | 881 | 163 | D163G | 0.922304842 | 0.015150837 |
| 841 | 277 | T277A | 0.914782215 | 0.018474111 | 882 | 287 | G287A | 0.922661653 | 0.021348893 |
| 842 | 62 | E62Q | 0.915179165 | 0.021308063 | 883 | 225 | P225A | 0.922854716 | 0.021349944 |
| 843 | 323 | K323Q | 0.915473325 | 0.036818475 | 884 | 127 | K127N | 0.922921698 | 0.036887547 |
| 844 | 313 | D313Y | 0.915477029 | 0.026066797 | 885 | 33 | K33E | 0.922921968 | 0.02135031 |
| 845 | 171 | A171S | 0.915905558 | 0.026069641 | 886 | 364 | F364Y | 0.922933927 | 0.026116159 |
| 846 | 328 | N328K | 0.916090379 | 0.018480365 | 887 | 59 | L59H | 0.92310104 | 0.036889205 |
| 847 | 52 | I52T | 0.916214092 | 0.021313723 | 888 | 39 | V39F | 0.923231718 | 0.026118124 |
| 848 | 165 | I165M | 0.916457119 | 0.0260733 | 889 | 109 | A109G | 0.923541509 | 0.015155728 |
| 849 | 27 | K27Q | 0.916732979 | 0.018483435 | 890 | 171 | A171V | 0.923877075 | 0.011798635 |
| 850 | 16 | K16R | 0.9167423 | 0.036830269 | 891 | 227 | V227D | 0.924077375 | 0.016584 |
| 851 | 24 | E24G | 0.916942954 | 0.036832133 | 892 | 277 | T277I | 0.924179306 | 0.036899172 |
| 852 | 288 | F288Y | 0.917075716 | 0.021318433 | 893 | 217 | I217Q | 0.924451425 | 0.018520206 |
| 853 | 255 | L255Q | 0.917111496 | 0.021318628 | 894 | 177 | M177T | 0.924654948 | 0.018521173 |
| 854 | 220 | A220T | 0.917242125 | 0.026078505 | 895 | 19 | H19P | 0.924685476 | 0.036903848 |
| 855 | 195 | F195C | 0.917337362 | 0.026079136 | 896 | 164 | F164S | 0.92479435 | 0.036904853 |
| 856 | 132 | T132R | 0.917985898 | 0.018489418 | 897 | 263 | G263C | 0.924797943 | 0.026128456 |
| 857 | 286 | Q286R | 0.918660249 | 0.036848075 | 898 | 223 | E223V | 0.925343193 | 0.018524442 |
| 858 | 92 | G92A | 0.918824199 | 0.011782582 | 899 | 216 | L216V | 0.925515205 | 0.021364408 |
| 900 | 48 | D48N | 0.925515979 | 0.018525263 | 941 | 165 | I165T | 0.931972641 | 0.026175639 |
| 901 | 217 | L217V | 0.925627237 | 0.018525791 | 942 | 2 | D2A | 0.932017566 | 0.026175933 |
| 902 | 229 | E229Q | 0.925767914 | 0.036913841 | 943 | 296 | Q296H | 0.932098375 | 0.018556445 |
| 903 | 295 | P295L | 0.925876322 | 0.036914842 | 944 | 109 | A109D | 0.932470501 | 0.036975549 |
| 904 | 113 | E113V | 0.92609559 | 0.021367559 | 945 | 114 | E114V | 0.932755254 | 0.026180771 |
| 905 | 10 | F10L | 0.926191024 | 0.026137636 | 946 | 355 | Q355H | 0.933036933 | 0.011291213 |
| 906 | 164 | F164I | 0.926271869 | 0.021368516 | 947 | 318 | K318E | 0.933711258 | 0.018564064 |
| 907 | 303 | I303V | 0.926343103 | 0.026138638 | 948 | 254 | V254I | 0.933888784 | 0.013193654 |
| 908 | 38 | S38G | 0.926485276 | 0.021369675 | 949 | 322 | D322V | 0.934363214 | 0.03699292 |
| 909 | 323 | K323E | 0.926637282 | 0.026140575 | 950 | 153 | A153S | 0.934624663 | 0.021413729 |
| 910 | 58 | N58T | 0.927601307 | 0.026146921 | 951 | 98 | C98Y | 0.934748373 | 0.021414396 |
| 911 | 297 | N297H | 0.927832765 | 0.026148443 | 952 | 215 | D215Y | 0.934842286 | 0.036997313 |
| 912 | 267 | M267T | 0.927848489 | 0.018536328 | 953 | 73 | I73N | 0.93509178 | 0.026196076 |
| 913 | 5 | R5T | 0.927877698 | 0.008890155 | 954 | 241 | N241T | 0.935214162 | 0.037000722 |
| 914 | 186 | V186A | 0.928070312 | 0.01853738 | 955 | 141 | N141H | 0.935230644 | 0.014091848 |
| 915 | 58 | N58I | 0.928290771 | 0.015174475 | 956 | 363 | K363N | 0.93537952 | 0.026197959 |
| 916 | 27 | K27T | 0.928330184 | 0.021379682 | 957 | 35 | V35L | 0.935420904 | 0.018572131 |
| 917 | 146 | A146P | 0.928494112 | 0.036938976 | 958 | 120 | T120P | 0.935594651 | 0.016633221 |
| 918 | 308 | G308A | 0.928566404 | 0.018539731 | 959 | 281 | I281V | 0.935968179 | 0.02620181 |
| 919 | 133 | K133 | 0.928625422 | 0.01854001 | 960 | 225 | P225S | 0.936036146 | 0.021421343 |
| 920 | 244 | P244T | 0.928751045 | 0.026154483 | 961 | 212 | V212I | 0.936095518 | 0.026202643 |
| 921 | 252 | G252V | 0.928931112 | 0.021382939 | 962 | 109 | A109S | 0.936267832 | 0.021422592 |
| 922 | 224 | C224G | 0.929024875 | 0.021383447 | 963 | 68 | D68G | 0.936402948 | 0.018576761 |
| 923 | 92 | G92S | 0.929351782 | 0.018543451 | 964 | 274 | K274Q | 0.936429731 | 0.026204829 |
| 924 | 81 | G81V | 0.929424333 | 0.036947541 | 965 | 221 | L221P | 0.936599174 | 0.021424378 |
| 925 | 202 | V202L | 0.929673473 | 0.036949834 | 966 | 215 | D215G | 0.936963807 | 0.02620832 |
| 926 | 168 | V168L | 0.929674474 | 0.036949844 | 967 | 30 | L30F | 0.937099176 | 0.018580042 |
| 927 | 290 | V290I | 0.929961668 | 0.018546338 | 968 | 24 | E24V | 0.937103721 | 0.015209109 |
| 928 | 104 | T104A | 0.930140769 | 0.026163615 | 969 | 64 | L64V | 0.937390097 | 0.02142864 |
| 929 | 185 | P185A | 0.930263031 | 0.018547765 | 970 | 100 | D100N | 0.937533666 | 0.026212044 |
| 930 | 240 | Y240H | 0.93027981 | 0.026164528 | 971 | 177 | M177V | 0.937781592 | 0.018583255 |
| 931 | 28 | E28Q | 0.930345836 | 0.018548156 | 972 | 128 | L128F | 0.938119935 | 0.02143257 |
| 932 | 345 | L345M | 0.93041652 | 0.016611138 | 973 | 92 | G92C | 0.938167122 | 0.01858507 |
| 933 | 36 | G36R | 0.93050642 | 0.018548916 | 974 | 160 | V160L | 0.938261917 | 0.037028626 |
| 934 | 198 | A198V | 0.930616429 | 0.016611992 | 975 | 198 | A198G | 0.938516966 | 0.021434708 |
| 935 | 272 | S272T | 0.930643828 | 0.018549566 | 976 | 28 | E28D | 0.938601129 | 0.016646007 |
| 936 | 333 | K333I | 0.930678502 | 0.021392402 | 977 | 87 | P87T | 0.938617343 | 0.018587189 |
| 937 | 341 | I341M | 0.930807516 | 0.0213931 | 978 | 147 | S147C | 0.938674845 | 0.021435558 |
| 938 | 263 | G263S | 0.930811855 | 0.036960306 | 979 | 268 | E268A | 0.939020417 | 0.018589086 |
| 939 | 280 | E280G | 0.931448788 | 0.016615547 | 980 | 5 | R5M | 0.939141343 | 0.016648302 |
| 940 | 274 | K274T | 0.931625177 | 0.026173359 | 981 | 290 | V290A | 0.939470057 | 0.037039671 |
| 982 | 85 | A85T | 0.939821425 | 0.018592853 | 1023 | 187 | D187E | 0.944831577 | 0.016672425 |
| 983 | 225 | P225T | 0.940320162 | 0.037047437 | 1024 | 317 | E317A | 0.944866421 | 0.026259831 |
| 984 | 247 | S247G | 0.940329202 | 0.021444456 | 1025 | 149 | V149L | 0.945001703 | 0.013232005 |
| 985 | 319 | R319G | 0.940392386 | 0.037048096 | 1026 | 62 | E62A | 0.945091919 | 0.03709094 |
| 986 | 19 | H19R | 0.940438815 | 0.026231006 | 1027 | 130 | S130C | 0.945185442 | 0.02147052 |
| 987 | 365 | L365W | 0.940701163 | 0.021446456 | 1028 | 265 | A265S | 0.94566961 | 0.037096196 |
| 988 | 307 | I307V | 0.940719946 | 0.021446557 | 1029 | 145 | D145E | 0.945833493 | 0.021473991 |
| 989 | 142 | L142V | 0.940749094 | 0.021446713 | 1030 | 202 | V202I | 0.945938883 | 0.0262668 |
| 990 | 223 | E223K | 0.940751387 | 0.021446726 | 1031 | 122 | N122H | 0.946167925 | 0.01862263 |
| 991 | 128 | L128R | 0.940777072 | 0.018597345 | 1032 | 82 | Q82E | 0.946174124 | 0.026268328 |
| 992 | 183 | S183A | 0.940831946 | 0.03705211 | 1033 | 64 | L64Q | 0.946266973 | 0.037101629 |
| 993 | 132 | T132I | 0.940894023 | 0.026233974 | 1034 | 267 | M267L | 0.94634685 | 0.026269449 |
| 994 | 257 | W257R | 0.941090214 | 0.026235253 | 1035 | 59 | L59F | 0.946403889 | 0.037102874 |
| 995 | 312 | G312R | 0.94109781 | 0.037054537 | 1036 | 130 | S130G | 0.946418581 | 0.037103008 |
| 996 | 135 | P135A | 0.941136199 | 0.021448794 | 1037 | 248 | I248F | 0.946690292 | 0.037105478 |
| 997 | 313 | D313A | 0.94125279 | 0.026236312 | 1038 | 145 | D145A | 0.946713328 | 0.016680382 |
| 998 | 276 | Q276K | 0.941308826 | 0.037056462 | 1039 | 16 | K16E | 0.946954586 | 0.026273395 |
| 999 | 87 | P87A | 0.941487755 | 0.021450682 | 1040 | 237 | S237G | 0.947271413 | 0.018627795 |
| 1000 | 204 | S204A | 0.941579378 | 0.021451174 | 1041 | 167 | D167E | 0.947286494 | 0.021481769 |
| 1001 | 38 | S38C | 0.941884947 | 0.037061719 | 1042 | 150 | Y150H | 0.947357187 | 0.0132401 |
| 1002 | 362 | K362R | 0.941962114 | 0.018602911 | 1043 | 286 | Q286L | 0.947396635 | 0.026276264 |
| 1003 | 207 | V207L | 0.942222218 | 0.026242628 | 1044 | 297 | N297Y | 0.947541982 | 0.026277207 |
| 1004 | 250 | V250I | 0.942424459 | 0.021455712 | 1045 | 95 | A95E | 0.947543805 | 0.021483146 |
| 1005 | 331 | S331Y | 0.942484381 | 0.021456034 | 1046 | 300 | K300N | 0.94765708 | 0.021483751 |
| 1006 | 139 | T139A | 0.942622671 | 0.021456776 | 1047 | 247 | S247T | 0.947709201 | 0.018629843 |
| 1007 | 3 | A3D | 0.942676001 | 0.015230905 | 1048 | 33 | K33Q | 0.947813224 | 0.018630329 |
| 1008 | 65 | A65D | 0.942733688 | 0.026245958 | 1049 | 123 | I123T | 0.947858614 | 0.018630541 |
| 1009 | 294 | E294G | 0.942791904 | 0.015231357 | 1050 | 269 | K269E | 0.94794221 | 0.013242108 |
| 1010 | 368 | H368P | 0.942863277 | 0.018607141 | 1051 | 251 | M251T | 0.948274057 | 0.016686974 |
| 1011 | 146 | A146G | 0.942876918 | 0.018607205 | 1052 | 153 | A153G | 0.948342508 | 0.026282399 |
| 1012 | 237 | S237I | 0.942943031 | 0.018607515 | 1053 | 230 | Y230F | 0.948386532 | 0.026282684 |
| 1013 | 139 | T139I | 0.943222594 | 0.037073915 | 1054 | 135 | P135R | 0.948392119 | 0.026282721 |
| 1014 | 164 | F164Y | 0.943337152 | 0.02146061 | 1055 | 164 | F164V | 0.948450989 | 0.026283102 |
| 1015 | 54 | N54Y | 0.943445184 | 0.015233907 | 1056 | 171 | A171P | 0.948627119 | 0.018634134 |
| 1016 | 184 | K184N | 0.94346195 | 0.01860995 | 1057 | 145 | D145Y | 0.948704648 | 0.021489353 |
| 1017 | 61 | R61G | 0.943616652 | 0.021462109 | 1058 | 328 | N328T | 0.948730702 | 0.018634618 |
| 1018 | 102 | V102M | 0.943654016 | 0.01861085 | 1059 | 270 | L270V | 0.948765843 | 0.037124333 |
| 1019 | 60 | V60M | 0.943734177 | 0.018611226 | 1060 | 25 | I25V | 0.948807387 | 0.026285413 |
| 1020 | 307 | I307L | 0.944200122 | 0.018613411 | 1061 | 121 | I121L | 0.94900844 | 0.018635916 |
| 1021 | 52 | I527 | 0.944219141 | 0.026255623 | 1062 | 189 | S189C | 0.94921066 | 0.026288027 |
| 1022 | 363 | K363R | 0.944667181 | 0.016671729 | 1063 | 266 | A266S | 0.949279565 | 0.037128995 |
| 1064 | 35 | V35G | 0.949600331 | 0.018638682 | 1105 | 31 | D31V | 0.954454341 | 0.021520026 |
| 1065 | 98 | C98S | 0.949834791 | 0.037134032 | 1106 | 312 | G312E | 0.954551115 | 0.021520541 |
| 1066 | 301 | M301T | 0.949963633 | 0.037135201 | 1107 | 270 | L270F | 0.954731155 | 0.018662608 |
| 1067 | 212 | V212L | 0.949966079 | 0.021496092 | 1108 | 237 | S237C | 0.954909324 | 0.014163536 |
| 1068 | 7 | V7M | 0.950056922 | 0.037136047 | 1109 | 268 | E268G | 0.955012452 | 0.01671536 |
| 1069 | 167 | D167N | 0.950080531 | 0.018640925 | 1110 | 324 | A324S | 0.955141013 | 0.026326379 |
| 1070 | 121 | I121M | 0.950238081 | 0.026294682 | 1111 | 74 | F74V | 0.955215407 | 0.026326859 |
| 1071 | 350 | T350I | 0.950433003 | 0.021498586 | 1112 | 314 | D314Y | 0.955317963 | 0.018665339 |
| 1072 | 295 | P295T | 0.950448686 | 0.018642643 | 1113 | 2 | D2V | 0.955355613 | 0.021524823 |
| 1073 | 358 | A358V | 0.950585413 | 0.021499399 | 1114 | 370 | L370I | 0.955451543 | 0.037184877 |
| 1074 | 190 | K190M | 0.950631236 | 0.018643496 | 1115 | 55 | N55S | 0.9554669 | 0.026328482 |
| 1075 | 126 | P1265 | 0.950633844 | 0.021499658 | 1116 | 25 | I25L | 0.955564402 | 0.021525934 |
| 1076 | 276 | Q276H | 0.95065036 | 0.026297352 | 1117 | 366 | E366D | 0.955614711 | 0.026329436 |
| 1077 | 287 | G287E | 0.950768845 | 0.018644138 | 1118 | 345 | L345P | 0.955939426 | 0.02633153 |
| 1078 | 3 | A3S | 0.950841255 | 0.021500765 | 1119 | 265 | A265P | 0.955974058 | 0.026331754 |
| 1079 | 215 | D215E | 0.950984396 | 0.013252542 | 1120 | 17 | L17V | 0.956074698 | 0.026332403 |
| 1080 | 127 | K127T | 0.951071576 | 0.018645551 | 1121 | 315 | A315P | 0.9561852 | 0.026333115 |
| 1081 | 58 | N58Y | 0.951189253 | 0.021502622 | 1122 | 333 | K333R | 0.956483683 | 0.02633504 |
| 1082 | 164 | F164C | 0.951332182 | 0.026301765 | 1123 | 363 | K363E | 0.95652276 | 0.018670943 |
| 1083 | 296 | Q296E | 0.95163624 | 0.021505007 | 1124 | 254 | V254L | 0.956595389 | 0.01528501 |
| 1084 | 118 | F118Y | 0.951751748 | 0.037151408 | 1125 | 369 | Q369P | 0.95683011 | 0.026337274 |
| 1085 | 142 | L142I | 0.951781604 | 0.026304673 | 1126 | 86 | V86A | 0.956865093 | 0.018672535 |
| 1086 | 40 | I40V | 0.951784531 | 0.026304692 | 1127 | 120 | T120I | 0.95707084 | 0.026338825 |
| 1087 | 218 | G218A | 0.951854804 | 0.037152341 | 1128 | 254 | V254A | 0.95742097 | 0.037202657 |
| 1088 | 96 | G96V | 0.952078108 | 0.026306591 | 1129 | 48 | D48V | 0.957426516 | 0.037202707 |
| 1089 | 267 | M267V | 0.952212962 | 0.021508083 | 1130 | 70 | Y70F | 0.957500825 | 0.037203377 |
| 1090 | 284 | N284D | 0.952221024 | 0.026307515 | 1131 | 268 | E268Q | 0.958052865 | 0.026345153 |
| 1091 | 88 | L88I | 0.952391066 | 0.021509032 | 1132 | 261 | N261T | 0.958116164 | 0.02634556 |
| 1092 | 167 | D167H | 0.952403012 | 0.037157306 | 1133 | 309 | N309T | 0.958142166 | 0.016728501 |
| 1093 | 8 | V8D | 0.953062745 | 0.037163277 | 1134 | 244 | P244Q | 0.958269745 | 0.021540314 |
| 1094 | 185 | P185L | 0.953084386 | 0.026313096 | 1135 | 269 | K269M | 0.95848191 | 0.026347916 |
| 1095 | 244 | 244A | 0.953247593 | 0.021513598 | 1136 | 92 | G92D | 0.958742688 | 0.015293314 |
| 1096 | 277 | T277S | 0.953376343 | 0.018656298 | 1137 | 152 | C152S | 0.958867181 | 0.013279487 |
| 1097 | 310 | A310V | 0.953806607 | 0.026317763 | 1138 | 141 | N141Y | 0.958893878 | 0.037215938 |
| 1098 | 5 | R5K | 0.953843602 | 0.021516773 | 1139 | 238 | S238Y | 0.958908311 | 0.018682026 |
| 1099 | 330 | L330M | 0.953850794 | 0.026318048 | 1140 | 218 | G218R | 0.958930139 | 0.02154382 |
| 1100 | 164 | F164L | 0.953852179 | 0.021516819 | 1141 | 9 | N9S | 0.958974716 | 0.037216667 |
| 1101 | 217 | L217P | 0.953886831 | 0.021517003 | 1142 | 232 | V232L | 0.958980954 | 0.021544089 |
| 1102 | 343 | A343P | 0.953899217 | 0.018658734 | 1143 | 258 | I258L | 0.959019105 | 0.018682541 |
| 1103 | 141 | N141S | 0.95402289 | 0.02631916 | 1144 | 284 | N284S | 0.959416685 | 0.026353933 |
| 1104 | 58 | N58H | 0.954061023 | 0.018659487 | 1145 | 256 | E256D | 0.959666914 | 0.026355543 |
| 1146 | 84 | S84N | 0.959882106 | 0.026356927 | 1187 | 218 | G218E | 0.964736049 | 0.018709029 |
| 1147 | 369 | Q369H | 0.959909501 | 0.026357103 | 1188 | 88 | L88S | 0.965067504 | 0.021576331 |
| 1148 | 146 | A146S | 0.960068632 | 0.026358127 | 1189 | 65 | A65G | 0.965296788 | 0.021577542 |
| 1149 | 231 | K231E | 0.960349249 | 0.018688712 | 1190 | 286 | Q286E | 0.965371228 | 0.02639217 |
| 1150 | 254 | V254G | 0.960395367 | 0.016737946 | 1191 | 296 | Q296K | 0.965393531 | 0.026392313 |
| 1151 | 368 | H368D | 0.960478449 | 0.015300018 | 1192 | 184 | K184R | 0.965398106 | 0.026392343 |
| 1152 | 253 | L253W | 0.9604852 | 0.026360806 | 1193 | 18 | P18S | 0.965573413 | 0.0187129 |
| 1153 | 256 | E256V | 0.960534533 | 0.016738529 | 1194 | 6 | Q6R | 0.965607577 | 0.016759741 |
| 1154 | 262 | G262E | 0.960549329 | 0.026361218 | 1195 | 360 | F360Y | 0.965617431 | 0.037276391 |
| 1155 | 306 | R306S | 0.960573863 | 0.03723107 | 1196 | 48 | D48A | 0.965628565 | 0.037276491 |
| 1156 | 233 | Q233K | 0.960844305 | 0.021553974 | 1197 | 146 | A146V | 0.965710173 | 0.026394342 |
| 1157 | 278 | I278M | 0.960963898 | 0.021554608 | 1198 | 273 | I273L | 0.965736257 | 0.018713653 |
| 1158 | 294 | E294V | 0.961004027 | 0.016740495 | 1199 | 332 | L332V | 0.965863021 | 0.037278594 |
| 1159 | 174 | V174A | 0.961198156 | 0.026365389 | 1200 | 141 | N141D | 0.96586447 | 0.021580542 |
| 1160 | 261 | N261I | 0.961298927 | 0.037237595 | 1201 | 168 | V168A | 0.966007208 | 0.021581296 |
| 1161 | 43 | S43C | 0.961321875 | 0.018693222 | 1202 | 232 | V232G | 0.966077393 | 0.021581667 |
| 1162 | 71 | K71R | 0.96133491 | 0.026366268 | 1203 | 53 | I53V | 0.966166399 | 0.016762074 |
| 1163 | 138 | S138C | 0.961436462 | 0.018693753 | 1204 | 59 | L59R | 0.966209321 | 0.02639754 |
| 1164 | 325 | L325P | 0.961443101 | 0.021557148 | 1205 | 277 | T277K | 0.966312363 | 0.0263982 |
| 1165 | 322 | D322G | 0.961582909 | 0.01530428 | 1206 | 328 | N328D | 0.966322884 | 0.016762727 |
| 1166 | 2 | D2G | 0.961605933 | 0.02155801 | 1207 | 233 | Q233H | 0.966336837 | 0.016762785 |
| 1167 | 358 | A358T | 0.961850181 | 0.01869567 | 1208 | 272 | S272C | 0.966344602 | 0.021583079 |
| 1168 | 106 | A106V | 0.962066233 | 0.021560449 | 1209 | 52 | I52S | 0.966363646 | 0.037283083 |
| 1169 | 269 | K269N | 0.962101804 | 0.018696836 | 1210 | 75 | L75Q | 0.966381894 | 0.021583276 |
| 1170 | 232 | V232E | 0.962108481 | 0.018696867 | 1211 | 130 | S130N | 0.966892162 | 0.016765102 |
| 1171 | 114 | E114G | 0.962220484 | 0.026371957 | 1212 | 327 | L327F | 0.967063306 | 0.037289354 |
| 1172 | 360 | F360I | 0.962258105 | 0.037246222 | 1213 | 141 | N141K | 0.967116658 | 0.026403349 |
| 1173 | 295 | P295A | 0.962273806 | 0.037246363 | 1214 | 322 | D322N | 0.96720097 | 0.021587601 |
| 1174 | 253 | L253V | 0.962450021 | 0.021562482 | 1215 | 138 | S138I | 0.967327291 | 0.018721002 |
| 1175 | 210 | V210G | 0.962586578 | 0.026374308 | 1216 | 314 | D314H | 0.967609245 | 0.014209335 |
| 1176 | 124 | V124I | 0.962752502 | 0.01869985 | 1217 | 151 | Y151H | 0.967799585 | 0.015328215 |
| 1177 | 123 | I123M | 0.963077613 | 0.02637746 | 1218 | 142 | L142R | 0.968112904 | 0.021592413 |
| 1178 | 207 | V207G | 0.963304694 | 0.015310919 | 1219 | 274 | K274N | 0.968237545 | 0.026410521 |
| 1179 | 278 | I278V | 0.963538479 | 0.016751098 | 1220 | 96 | G96A | 0.968453021 | 0.016771609 |
| 1180 | 318 | K318I | 0.963632924 | 0.021568743 | 1221 | 40 | L40F | 0.968487809 | 0.015330859 |
| 1181 | 246 | F246S | 0.963977579 | 0.026383235 | 1222 | 234 | A234S | 0.968570583 | 0.021594827 |
| 1182 | 113 | E113D | 0.964097785 | 0.026384006 | 1223 | 184 | K184E | 0.968752037 | 0.037304477 |
| 1183 | 3 | A3T | 0.964230445 | 0.01870669 | 1224 | 58 | N58S | 0.968853459 | 0.016773277 |
| 1184 | 27 | K27M | 0.964585081 | 0.015315851 | 1225 | 182 | L182V | 0.968947549 | 0.026415061 |
| 1185 | 46 | S46L | 0.964678512 | 0.037267966 | 1226 | 294 | E294K | 0.968976012 | 0.037306482 |
| 1186 | 327 | L327R | 0.964730101 | 0.016756077 | 1227 | 301 | M301K | 0.969111308 | 0.021597678 |
| 1228 | 12 | P12H | 0.969177922 | 0.01872954 | 1269 | 218 | G218V | 0.97360174 | 0.021621316 |
| 1229 | 327 | L327V | 0.969395579 | 0.037310236 | 1270 | 36 | G36D | 0.973627162 | 0.026444929 |
| 1230 | 362 | K362T | 0.96983496 | 0.021601492 | 1271 | 341 | I341F | 0.973630112 | 0.021621465 |
| 1231 | 91 | I91L | 0.969849762 | 0.011401715 | 1272 | 268 | E268D | 0.973715537 | 0.026445492 |
| 1232 | 81 | G81A | 0.969923352 | 0.026421297 | 1273 | 142 | L142F | 0.97382194 | 0.016793942 |
| 1233 | 73 | I73T | 0.969997313 | 0.021602348 | 1274 | 308 | G308R | 0.973831603 | 0.018750966 |
| 1234 | 351 | I351S | 0.970045827 | 0.02642208 | 1275 | 315 | A315S | 0.973903844 | 0.021622904 |
| 1235 | 362 | K362Q | 0.97012301 | 0.02160301 | 1276 | 31 | D31N | 0.974205209 | 0.026448612 |
| 1236 | 40 | L40H | 0.970328068 | 0.015337923 | 1277 | 264 | A264P | 0.974207897 | 0.01535279 |
| 1237 | 330 | L330V | 0.970349507 | 0.026424019 | 1278 | 306 | R306L | 0.974308562 | 0.018753159 |
| 1238 | 26 | Q26L | 0.970375164 | 0.026424183 | 1279 | 47 | S47A | 0.974403952 | 0.021625532 |
| 1239 | 309 | N309Y | 0.970416492 | 0.018735249 | 1280 | 34 | G34V | 0.974438845 | 0.0264501 |
| 1240 | 212 | V212A | 0.97059115 | 0.015338932 | 1281 | 211 | I211V | 0.974452842 | 0.026450189 |
| 1241 | 266 | A266D | 0.970608382 | 0.026425672 | 1282 | 258 | I258N | 0.974554342 | 0.021626322 |
| 1242 | 308 | G308S | 0.970934915 | 0.026427757 | 1283 | 223 | E223Q | 0.974743837 | 0.018755159 |
| 1243 | 360 | F360C | 0.970975866 | 0.021607502 | 1284 | 284 | N284H | 0.974751765 | 0.021627359 |
| 1244 | 111 | A111G | 0.970984987 | 0.026428077 | 1285 | 50 | A50S | 0.974759614 | 0.0216274 |
| 1245 | 68 | D68H | 0.971110932 | 0.021608213 | 1286 | 295 | P295S | 0.974804922 | 0.018755439 |
| 1246 | 247 | S247N | 0.9711902 | 0.018738813 | 1287 | 6 | Q6E | 0.974860759 | 0.021627931 |
| 1247 | 50 | A50D | 0.971252901 | 0.037326841 | 1288 | 358 | A358G | 0.974997578 | 0.016798823 |
| 1248 | 331 | S331P | 0.971301893 | 0.0264301 | 1289 | 321 | L321H | 0.975017204 | 0.026453783 |
| 1249 | 363 | K363Q | 0.971461518 | 0.021610059 | 1290 | 214 | D214A | 0.975214151 | 0.021629787 |
| 1250 | 326 | E326A | 0.971545933 | 0.037329459 | 1291 | 205 | A205S | 0.975263607 | 0.037362626 |
| 1251 | 362 | K362N | 0.971653057 | 0.021611067 | 1292 | 188 | V188I | 0.975408366 | 0.037363916 |
| 1252 | 33 | K33N | 0.971679124 | 0.026432507 | 1293 | 240 | Y240F | 0.975556769 | 0.01535795 |
| 1253 | 229 | E229V | 0.971693711 | 0.018741131 | 1294 | 273 | 1273M | 0.975954754 | 0.016802793 |
| 1254 | 249 | Y249D | 0.971718148 | 0.026432756 | 1295 | 118 | F118L | 0.976029238 | 0.037369446 |
| 1255 | 261 | N261K | 0.971753821 | 0.021611597 | 1296 | 313 | D313E | 0.97629121 | 0.026461891 |
| 1256 | 27 | K27N | 0.971999645 | 0.026434552 | 1297 | 314 | D314G | 0.976428043 | 0.016804756 |
| 1257 | 170 | G170R | 0.97200195 | 0.01874255 | 1298 | 36 | G36A | 0.976466976 | 0.026463009 |
| 1258 | 311 | K311N | 0.972019864 | 0.018742633 | 1299 | 273 | I273V | 0.976531989 | 0.021636703 |
| 1259 | 220 | A220S | 0.972175694 | 0.021613817 | 1300 | 122 | N122T | 0.976605465 | 0.037374576 |
| 1260 | 4 | P4A | 0.972313959 | 0.018743986 | 1301 | 319 | R319I | 0.976687564 | 0.037375307 |
| 1261 | 278 | I278L | 0.972344813 | 0.021614707 | 1302 | 65 | A65S | 0.976710476 | 0.021637639 |
| 1262 | 112 | A112V | 0.972438958 | 0.026437354 | 1303 | 291 | C291G | 0.976716569 | 0.015362383 |
| 1263 | 273 | I273N | 0.972494508 | 0.026437709 | 1304 | 34 | G34R | 0.976771623 | 0.02163796 |
| 1264 | 132 | T132P | 0.9725805 | 0.037338698 | 1305 | 219 | F219Y | 0.977014955 | 0.037378221 |
| 1265 | 221 | L221F | 0.972600757 | 0.018745306 | 1306 | 271 | S271R | 0.977037993 | 0.02646664 |
| 1266 | 237 | S237T | 0.972757277 | 0.018746026 | 1307 | 276 | Q276R | 0.977496562 | 0.026469555 |
| 1267 | 214 | D214H | 0.973518447 | 0.018749527 | 1308 | 42 | M42L | 0.977536752 | 0.02646981 |
| 1268 | 142 | L142P | 0.973553855 | 0.037347383 | 1309 | 339 | G339R | 0.977719773 | 0.014245541 |
| 1310 | 57 | E57Q | 0.977731391 | 0.021642993 | 1351 | 365 | L365S | 0.981898968 | 0.018787956 |
| 1311 | 168 | V168G | 0.977888448 | 0.021643816 | 1352 | 322 | D322A | 0.981958116 | 0.026497869 |
| 1312 | 18 | P18L | 0.977894788 | 0.037386048 | 1353 | 280 | E280D | 0.98217378 | 0.021666245 |
| 1313 | 169 | K169N | 0.977910932 | 0.021643934 | 1354 | 273 | I273F | 0.98222007 | 0.037424458 |
| 1314 | 322 | D322E | 0.978035729 | 0.037387301 | 1355 | 110 | K110R | 0.98240174 | 0.016829475 |
| 1315 | 310 | A310P | 0.978123467 | 0.021645048 | 1356 | 27 | K27E | 0.982403881 | 0.021667447 |
| 1316 | 113 | E113A | 0.978488422 | 0.026475857 | 1357 | 161 | E161A | 0.982483562 | 0.026501198 |
| 1317 | 66 | V66I | 0.978716686 | 0.037393356 | 1358 | 201 | N201S | 0.982532222 | 0.026501506 |
| 1318 | 259 | K259I | 0.978770474 | 0.037393834 | 1359 | 219 | F219L | 0.98263068 | 0.037428098 |
| 1319 | 331 | S331A | 0.978829782 | 0.026478024 | 1360 | 193 | V193A | 0.98288426 | 0.016831467 |
| 1320 | 260 | N260H | 0.978867754 | 0.021648947 | 1361 | 239 | L239W | 0.983193617 | 0.026505694 |
| 1321 | 29 | L29S | 0.978935885 | 0.015370858 | 1362 | 363 | K363T | 0.983208619 | 0.018793943 |
| 1322 | 123 | I123L | 0.978984787 | 0.02164956 | 1363 | 269 | K269T | 0.983249251 | 0.018794129 |
| 1323 | 52 | I52F | 0.979007591 | 0.037395941 | 1364 | 248 | I248V | 0.983566643 | 0.018795579 |
| 1324 | 337 | S337T | 0.979037676 | 0.018774859 | 1365 | 143 | N143D | 0.983723673 | 0.02650905 |
| 1325 | 367 | M367R | 0.979054773 | 0.026479453 | 1366 | 31 | D31G | 0.983971315 | 0.018797427 |
| 1326 | 90 | L90V | 0.979156576 | 0.018775404 | 1367 | 355 | Q355L | 0.983996355 | 0.021675765 |
| 1327 | 231 | K231Q | 0.979219254 | 0.026480497 | 1368 | 280 | E280Q | 0.984054349 | 0.026511142 |
| 1328 | 219 | F219V | 0.979409728 | 0.016817106 | 1369 | 196 | A196V | 0.984571077 | 0.018800166 |
| 1329 | 127 | K127Q | 0.979530853 | 0.026482475 | 1370 | 93 | L93W | 0.984682886 | 0.026515119 |
| 1330 | 214 | D214G | 0.979975611 | 0.018779155 | 1371 | 257 | W257L | 0.984739984 | 0.021679646 |
| 1331 | 126 | P126H | 0.980115228 | 0.016820024 | 1372 | 323 | K323T | 0.984908403 | 0.026516545 |
| 1332 | 324 | A324T | 0.980142901 | 0.016820139 | 1373 | 82 | Q82H | 0.985048176 | 0.026517429 |
| 1333 | 194 | I194T | 0.98024432 | 0.037406927 | 1374 | 250 | V250L | 0.985149099 | 0.018802804 |
| 1334 | 249 | Y249S | 0.980314423 | 0.021656521 | 1375 | 272 | S272Y | 0.985169419 | 0.021681886 |
| 1335 | 76 | Q76P | 0.980397782 | 0.021656957 | 1376 | 252 | G252D | 0.985318806 | 0.02651914 |
| 1336 | 37 | I37S | 0.980501611 | 0.01537683 | 1377 | 313 | D313H | 0.985344068 | 0.021682797 |
| 1337 | 145 | D145N | 0.980545726 | 0.026488914 | 1378 | 304 | P304R | 0.985427785 | 0.026519829 |
| 1338 | 77 | G77A | 0.980558228 | 0.026488994 | 1379 | 270 | L270H | 0.985433163 | 0.021683262 |
| 1339 | 314 | D314E | 0.980759579 | 0.016822689 | 1380 | 6 | Q6P | 0.985707924 | 0.016843116 |
| 1340 | 294 | E294Q | 0.980771396 | 0.018782798 | 1381 | 264 | A264S | 0.985742049 | 0.026521815 |
| 1341 | 127 | K127E | 0.980992864 | 0.016823653 | 1382 | 211 | I211M | 0.985830839 | 0.026522377 |
| 1342 | 313 | D313G | 0.981103048 | 0.018784315 | 1383 | 128 | L128 | 0.985940098 | 0.015397528 |
| 1343 | 138 | S1387 | 0.98122489 | 0.026493221 | 1384 | 309 | N309K | 0.985996572 | 0.026523424 |
| 1344 | 324 | A324G | 0.981283213 | 0.037416148 | 1385 | 86 | V86I | 0.986045311 | 0.026523732 |
| 1345 | 48 | D48H | 0.981476478 | 0.037417863 | 1386 | 122 | N122D | 0.986071984 | 0.016844616 |
| 1346 | 240 | Y240C | 0.981567088 | 0.021663073 | 1387 | 55 | N55Y | 0.986248105 | 0.026525013 |
| 1347 | 68 | D68N | 0.981590156 | 0.013356444 | 1388 | 367 | M367V | 0.987121212 | 0.018811798 |
| 1348 | 129 | G129A | 0.981631029 | 0.026495796 | 1389 | 19 | H19N | 0.987349954 | 0.021693252 |
| 1349 | 281 | I281L | 0.981743666 | 0.021663997 | 1390 | 172 | V172E | 0.987364171 | 0.018812905 |
| 1350 | 202 | V202F | 0.98174705 | 0.037420263 | 1391 | 368 | H368N | 0.987514919 | 0.026533014 |
| 1392 | 184 | K184T | 0.987534829 | 0.037471499 | | 132 | T132K | 0.993127268 | 0.021723287 |
| 1393 | 16 | K16T | 0.987738912 | 0.021695278 | | 331 | S331F | 0.993168169 | 0.037521172 |
| 1394 | 136 | D136N | 0.987754971 | 0.018814686 | | 369 | Q369E | 0.99350776 | 0.016875185 |
| 1395 | 97 | R97K | 0.987884712 | 0.021696037 | | 227 | V227L | 0.99358911 | 0.013396665 |
| 1396 | 266 | A266T | 0.987892893 | 0.021696079 | | 261 | N261Y | 0.993641143 | 0.02657161 |
| 1397 | 336 | R336T | 0.988368076 | 0.026538399 | | 128 | L128H | 0.993721624 | 0.026572116 |
| 1398 | 260 | N260I | 0.988502797 | 0.016854625 | | 333 | K333T | 0.993876789 | 0.014302941 |
| 1399 | 260 | N260Y | 0.98854398 | 0.037480411 | | 356 | K356N | 0.994082703 | 0.037529218 |
| 1400 | 2 | D2N | 0.988667908 | 0.018818844 | | 336 | R336M | 0.994151665 | 0.015428649 |
| 1401 | 272 | S272F | 0.988773246 | 0.021700662 | | 231 | K231R | 0.994238601 | 0.026575366 |
| 1402 | 351 | 351L | 0.988892704 | 0.021701284 | | 28 | E28K | 0.994265825 | 0.018844288 |
| 1403 | 329 | M329L | 0.989137241 | 0.037485648 | | 310 | A310D | 0.994365521 | 0.037531706 |
| 1404 | 321 | L321I | 0.98929897 | 0.037487075 | | 10 | F10Y | 0.994404932 | 0.037532052 |
| 1405 | 343 | A343V | 0.989790822 | 0.026547371 | | 227 | V227A | 0.994472152 | 0.016879139 |
| 1406 | 129 | G129E | 0.989821963 | 0.018824097 | | 125 | H125N | 0.994754107 | 0.021731725 |
| 1407 | 54 | N54K | 0.98989876 | 0.021706517 | | 27 | K27R | 0.994795256 | 0.021731938 |
| | 223 | E223A | 0.990033162 | 0.018825058 | | 260 | N260T | 0.99484512 | 0.016880668 |
| | 196 | A196G | 0.99011522 | 0.014289627 | | 354 | V354I | 0.995093276 | 0.021733483 |
| | 84 | S84G | 0.990154639 | 0.037494623 | | 260 | N260K | 0.995130659 | 0.037538432 |
| | 256 | E256A | 0.990254121 | 0.037495501 | | 265 | A265E | 0.995286556 | 0.021734484 |
| | 367 | M367 | 0.990359821 | 0.026550957 | | 343 | A343T | 0.995320861 | 0.026582165 |
| | 297 | N297S | 0.990361482 | 0.026550968 | | 348 | A348T | 0.99545363 | 0.037541271 |
| | 52 | I52V | 0.990581135 | 0.037498384 | | 172 | V172A | 0.995537836 | 0.015433887 |
| | 326 | E326K | 0.990754412 | 0.037499912 | | 26 | Q26H | 0.995543497 | 0.026583563 |
| | 349 | V349A | 0.990977982 | 0.026554851 | | 162 | F162Y | 0.995544999 | 0.026583573 |
| | 25 | I25T | 0.991148753 | 0.037503388 | | 367 | M367K | 0.995546683 | 0.037542088 |
| | 338 | V338L | 0.991158727 | 0.018830177 | | 314 | D314N | 0.995636832 | 0.016883911 |
| | 350 | T350K | 0.991194569 | 0.026556215 | | 24 | E24Q | 0.995711118 | 0.026584616 |
| | 167 | D167A | 0.991336104 | 0.014293952 | | 318 | K318N | 0.995738011 | 0.037543769 |
| | 205 | A205P | 0.991416465 | 0.037505747 | | 88 | L88V | 0.995831035 | 0.014309846 |
| | 132 | T132A | 0.991604997 | 0.018832205 | | 125 | H125Q | 0.995840538 | 0.021737354 |
| | 195 | F195V | 0.991667078 | 0.021715707 | | 136 | D136V | 0.995844211 | 0.037544702 |
| | 352 | E352G | 0.991788577 | 0.026559955 | | 260 | N260S | 0.995898012 | 0.021737652 |
| | 75 | L75M | 0.992230881 | 0.021718635 | | 226 | S226P | 0.996119415 | 0.03754712 |
| | 8 | V8G | 0.992633003 | 0.021720722 | | 303 | I303L | 0.996126604 | 0.037547183 |
| | 214 | D214Y | 0.992738508 | 0.02172127 | | 189 | S189T | 0.996146971 | 0.026587352 |
| | 34 | G34E | 0.992831769 | 0.02656652 | | 68 | D68V | 0.996243831 | 0.02658796 |
| | 325 | L325R | 0.992832723 | 0.026566526 | | 313 | D313N | 0.996280915 | 0.015436693 |
| | 265 | A265G | 0.992875406 | 0.016872591 | | 265 | A265V | 0.996473121 | 0.018854295 |
| | 129 | G129V | 0.993032622 | 0.01883869 | | 229 | E229K | 0.996514212 | 0.037550587 |
| | 2 | DZE | 0.993060008 | 0.026567956 | | 272 | S272A | 0.996764789 | 0.018855617 |
| | 249 | Y249H | 0.996892589 | 0.021742802 | | 5 | RSW | 1.002258214 | 0.0143325 |
| | 219 | F219S | 0.996956876 | 0.021743135 | | 214 | D214E | 1.002582162 | 0.026627658 |
| | 238 | S238T | 0.99705585 | 0.021743647 | | 157 | V157A | 1.002588522 | 0.015460462 |
| | 83 | F83S | 0.997546968 | 0.037559652 | | 281 | 1281M | 1.002898651 | 0.021773831 |
| | 141 | N14I | 0.997785826 | 0.026597633 | | 315 | A315T | 1.003132357 | 0.021775036 |
| | 234 | A234G | 0.998175743 | 0.021749441 | | 360 | F360V | 1.003280533 | 0.026632022 |
| | 228 | L228M | 0.998464981 | 0.021750937 | | 38 | S38T | 1.003439846 | 0.026633017 |
| | 368 | H368Q | 0.998672035 | 0.03756952 | | 231 | K231M | 1.003459041 | 0.037611425 |
| | 308 | G308V | 0.998697165 | 0.037569741 | | 267 | M267I | 1.003568875 | 0.021777286 |
| | 315 | A315V | 0.99901504 | 0.026605337 | | 304 | P304L | 1.003668419 | 0.021777799 |
| | 86 | V86L | 0.999122982 | 0.026606013 | | 259 | K259N | 1.0041708 | 0.021780388 |
| | 112 | A112G | 0.999316722 | 0.037575172 | | 177 | M177L | 1.004333749 | 0.026638599 |
| | 114 | E114D | 0.99932122 | 0.016898986 | | 309 | N309S | 1.004598865 | 0.021782593 |
| | 137 | P137A | 0.999368729 | 0.021755609 | | 313 | D313V | 1.004709801 | 0.026640946 |
| | 275 | S275A | 0.999372057 | 0.026607573 | | 143 | N143I | 1.00514833 | 0.021785423 |
| | 301 | M301V | 0.99941818 | 0.037576061 | | 258 | 1258V | 1.005251648 | 0.026644328 |
| | 259 | K259Q | 0.999437517 | 0.018867713 | | 286 | Q286K | 1.005508374 | 0.018895114 |
| | 137 | P137L | 0.99961854 | 0.014323206 | | 295 | P295H | 1.005509021 | 0.02178728 |
| | 264 | A264T | 0.99988895 | 0.02661081 | | 47 | S47P | 1.005574795 | 0.018895414 |
| | 114 | E114D | 1.000067518 | 0.02175922 | | 223 | E223D | 1.005656346 | 0.021788038 |
| | 235 | G235R | 1.000216176 | 0.026612859 | | 252 | G252S | 1.005871443 | 0.01889675 |
| | 314 | D314V | 1.000224746 | 0.026612912 | | 238 | S238C | 1.006267512 | 0.016927312 |
| | 330 | L330W | 1.000266154 | 0.03758349 | | 134 | 1134V | 1.006426909 | 0.016927961 |
| | 210 | V210M | 1.000297948 | 0.026613371 | | 251 | M251V | 1.006564647 | 0.026652516 |
| | 326 | E326V | 1.000315105 | 0.018871681 | | 219 | F219C | 1.006615887 | 0.021792975 |
| | 370 | L370V | 1.00051742 | 0.021761543 | | 69 | N69T | 1.006634372 | 0.026652951 |
| | 195 | F195Y | 1.000647454 | 0.018873183 | | 261 | N261D | 1.006935188 | 0.015476789 |
| | 266 | A266G | 1.000715785 | 0.021762568 | | 256 | E256G | 1.00742426 | 0.01890374 |
| | 214 | D214N | 1.000750455 | 0.018873648 | | 251 | M251K | 1.007607204 | 0.037647628 |
| | 133 | K133N | 1.000754538 | 0.026616228 | | 367 | M367T | 1.007743764 | 0.026659863 |
| | 112 | A112S | 1.001081676 | 0.021764457 | | 350 | T350R | 1.007758465 | 0.021798851 |
| | 185 | P185R | 1.001231407 | 0.018875821 | | 90 | L90I | 1.007782317 | 0.026660104 |
| | 122 | N122K | 1.00124738 | 0.037592081 | | 309 | N309H | 1.007793612 | 0.037649252 |
| | 95 | A95V | 1.001287086 | 0.02661956 | | 143 | N143Y | 1.007936657 | 0.037650499 |
| | 248 | I248T | 1.001292924 | 0.021765547 | | 173 | L173V | 1.00798562 | 0.012058931 |
| | 118 | F118I | 1.001405025 | 0.021766126 | | 171 | A171G | 1.008219172 | 0.013445326 |
| | 35 | V35D | 1.001609427 | 0.021767181 | | 110 | K110Q | 1.008255193 | 0.018907478 |
| | 207 | V207D | 1.001649407 | 0.0375956 | | 232 | V232M | 1.00828122 | 0.021801537 |
| | 226 | S226T | 1.001699632 | 0.016908698 | | 304 | P304T | 1.008412658 | 0.016936036 |
| | 233 | Q233L | 1.002088286 | 0.018879691 | | 137 | P137S | 1.008538511 | 0.018908752 |
| | 95 | A95T | 1.002202792 | 0.026625287 | | 330 | L330S | 1.008756008 | 0.015483616 |
| | 69 | N69H | 1.009192502 | 0.026668883 | | 255 | L255V | 1.015280596 | 0.021837423 |
| | 318 | K318T | 1.009315478 | 0.02180685 | | 180 | N180T | 1.015371207 | 0.018939408 |
| | 122 | N122S | 1.009415866 | 0.037663381 | | 234 | A234D | 1.015432685 | 0.03771565 |
| | 336 | R336W | 1.00988498 | 0.026673191 | | 33 | K33T | 1.01589028 | 0.018941732 |
| | 326 | E326D | 1.010133876 | 0.03766963 | | 269 | K269R | 1.016062273 | 0.01696705 |
| | 295 | P295R | 1.010142444 | 0.01891596 | | 133 | K133T | 1.01618493 | 0.03772217 |
| | 85 | A85D | 1.010160982 | 0.037669865 | | 6 | Q6K | 1.016311128 | 0.014381734 |
| | 346 | Y346F | 1.010186232 | 0.018916157 | | 359 | A359G | 1.016563996 | 0.037725454 |
| | 271 | S271T | 1.010200943 | 0.021811396 | | 234 | A234T | 1.016711758 | 0.016969677 |
| | 322 | D322H | 1.010346482 | 0.037671479 | | 310 | A310G | 1.016755295 | 0.021844963 |
| | 208 | T208S | 1.01037193 | 0.021812273 | | 284 | N284K | 1.016797263 | 0.037727475 |
| | 239 | L239S | 1.01078641 | 0.0218144 | | 2 | D2H | 1.016846839 | 0.026716396 |
| | 209 | V209L | 1.010967239 | 0.021815327 | | 172 | V172I | 1.017230694 | 0.013475096 |
| | 316 | L316I | 1.011309087 | 0.026682045 | | 349 | V349I | 1.017245555 | 0.018947795 |
| | 208 | T208A | 1.011326382 | 0.026682153 | | 37 | I37V | 1.017291781 | 0.026719151 |
| | 348 | A348P | 1.011339216 | 0.037680112 | | 130 | S130I | 1.017310676 | 0.026719268 |
| | 36 | G36S | 1.011406139 | 0.026682648 | | 358 | A358S | 1.017313849 | 0.026719288 |
| | 357 | L357V | 1.011518768 | 0.016948647 | | 350 | T350S | 1.017471999 | 0.026720266 |
| | 352 | E352K | 1.011609079 | 0.037682458 | | 349 | V349F | 1.017603503 | 0.021849297 |
| | 226 | S226W | 1.0116545 | 0.026684192 | | 116 | K116N | 1.017761823 | 0.018950103 |
| | 35 | V35A | 1.012022131 | 0.015495843 | | 232 | V232A | 1.017774227 | 0.021850169 |
| | 88 | L88F | 1.012165585 | 0.014367252 | | 270 | L270I | 1.017882882 | 0.037736875 |
| | 336 | R336G | 1.012188932 | 0.037687497 | | 208 | T208N | 1.017941818 | 0.026723174 |
| | 42 | M42K | 1.012351839 | 0.037688913 | | 29 | L29F | 1.018299764 | 0.026725388 |
| | 120 | T120N | 1.012676624 | 0.018927334 | | 125 | H125Y | 1.018594911 | 0.018953827 |
| | 351 | I351N | 1.013002483 | 0.018928795 | | 370 | L370Q | 1.018777756 | 0.026728345 |
| | 38 | S38N | 1.013736316 | 0.021829519 | | 252 | G252C | 1.018951311 | 0.03774612 |
| | 325 | L325F | 1.013819434 | 0.026697633 | | 294 | E294A | 1.019001654 | 0.021856435 |
| | 22 | L22F | 1.013853118 | 0.037701948 | | 311 | K311R | 1.019003207 | 0.021856443 |
| | 54 | N54S | 1.014067611 | 0.018933569 | | 348 | A348S | 1.019183257 | 0.026730852 |
| | 26 | Q26E | 1.014099337 | 0.026699369 | | 91 | 191M | 1.019364244 | 0.037749691 |
| | 264 | A264V | 1.014194467 | 0.021831865 | | 20 | S20A | 1.020002122 | 0.037755205 |
| | 296 | Q296R | 1.014625933 | 0.026702635 | | 236 | N236Y | 1.020018199 | 0.021861622 |
| | 115 | A115S | 1.014798588 | 0.026703705 | | 326 | E326G | 1.020375575 | 0.021863444 |
| | 135 | P135Q | 1.014875859 | 0.026704184 | | 173 | L173M | 1.02045417 | 0.013485708 |
| | 304 | P304Q | 1.014890704 | 0.026704277 | | 312 | G312A | 1.020539496 | 0.02186428 |
| | 323 | K323I | 1.014910837 | 0.021835531 | | 26 | Q26P | 1.020561349 | 0.018962608 |
| | 365 | L365M | 1.014953213 | 0.026704664 | | 251 | M251I | 1.020782086 | 0.026740732 |
| | 227 | V227G | 1.015055431 | 0.015507177 | | 337 | S337C | 1.020957031 | 0.037763456 |
| | 367 | M367L | 1.015125272 | 0.016963259 | | 370 | L370R | 1.020959287 | 0.016986829 |
| | 93 | L93V | 1.015243947 | 0.026706466 | | 151 | Y151F | 1.021238574 | 0.018965629 |
| | 315 | A315D | 1.021471419 | 0.018966668 | | 26 | Q26K | 1.028001163 | 0.026785213 |
| | 236 | N236T | 1.021566642 | 0.037768721 | | 261 | N261S | 1.028070346 | 0.026785639 |
| | 145 | D145H | 1.021576401 | 0.015531473 | | 259 | K259E | 1.028199156 | 0.037825863 |
| | 163 | D163N | 1.02161287 | 0.03776912 | | 205 | A205G | 1.028274835 | 0.037826514 |
| | 211 | I211L | 1.021648341 | 0.015531741 | | 96 | G96E | 1.028479495 | 0.017017089 |
| | 48 | D48E | 1.021709446 | 0.037769954 | | 188 | V188L | 1.028484262 | 0.037828314 |
| | 62 | E62K | 1.02174579 | 0.021870428 | | 228 | L228V | 1.028524957 | 0.037828664 |
| | 143 | N143H | 1.021821258 | 0.016990304 | | 32 | Y32S | 1.028838335 | 0.037831357 |
| | 194 | I194L | 1.02206581 | 0.026748657 | | 194 | I194M | 1.02898795 | 0.037832642 |
| | 259 | K259R | 1.022134657 | 0.026749082 | | 331 | S331C | 1.02900297 | 0.019000183 |
| | 30 | L30V | 1.022650843 | 0.021875037 | | 338 | V338M | 1.029103647 | 0.037833636 |
| | 32 | Y32N | 1.023049375 | 0.021877066 | | 172 | V172L | 1.029363589 | 0.021909144 |
| | 243 | P243S | 1.02315868 | 0.01897419 | | 23 | L23V | 1.029376455 | 0.026793664 |
| | 51 | K51E | 1.023725722 | 0.018976716 | | 95 | A95S | 1.029501078 | 0.026794429 |
| | 345 | L345V | 1.023945736 | 0.018977696 | | 290 | V290F | 1.029501967 | 0.037837058 |
| | 150 | Y150F | 1.024514851 | 0.015542391 | | 228 | L228Q | 1.029615795 | 0.019002903 |
| | 106 | A106S | 1.024713741 | 0.021885534 | | 71 | K71M | 1.029662781 | 0.037838439 |
| | 6 | Q6L | 1.024778709 | 0.017002215 | | 51 | K51N | 1.029818866 | 0.021911452 |
| | 301 | M30I | 1.024888623 | 0.02676606 | | 195 | F195L | 1.030131835 | 0.015563209 |
| | 22 | L22V | 1.024902166 | 0.017002711 | | 210 | V210L | 1.030693594 | 0.037847289 |
| | 294 | E294D | 1.025072456 | 0.037798956 | | 245 | C245S | 1.030718844 | 0.019007796 |
| | 95 | A95G | 1.025144126 | 0.037799574 | | 238 | S238A | 1.03079243 | 0.037848137 |
| | 50 | A50T | 1.025209416 | 0.015544969 | | 311 | K311T | 1.030875764 | 0.017026702 |
| | 55 | N55C | 1.025285393 | 0.017004253 | | 159 | G159S | 1.030908335 | 0.037849132 |
| | 203 | G203S | 1.025430053 | 0.026769395 | | 351 | I351F | 1.031011981 | 0.026803704 |
| | 315 | A315G | 1.025745419 | 0.037804752 | | 113 | E113Q | 1.031237016 | 0.01702815 |
| | 153 | A153E | 1.025857126 | 0.018986203 | | 64 | L64I | 1.031254962 | 0.021918728 |
| | 7 | V7L | 1.025939044 | 0.021891762 | | 97 | R97W | 1.03130465 | 0.019010394 |
| | 246 | F246I | 1.025989812 | 0.018986793 | | 222 | R222Q | 1.031766403 | 0.019012441 |
| | 42 | M42T | 1.026000582 | 0.018986841 | | 177 | M177I | 1.031787057 | 0.026808458 |
| | 332 | L332M | 1.026025785 | 0.018986953 | | 85 | A85S | 1.031834154 | 0.02192166 |
| | 204 | S204T | 1.026107163 | 0.026773563 | | 253 | L253F | 1.031888534 | 0.037857541 |
| | 300 | K300Q | 1.026117978 | 0.018987363 | | 356 | K356E | 1.032238467 | 0.021923707 |
| | 311 | K311E | 1.026218383 | 0.026774248 | | 180 | N180S | 1.032283615 | 0.019014733 |
| | 165 | I165V | 1.026317513 | 0.017008403 | | 53 | I53L | 1.0324121 | 0.01557164 |
| | 116 | K116M | 1.026494629 | 0.037811202 | | 231 | K231T | 1.033469359 | 0.021929934 |
| | 110 | K110N | 1.026656121 | 0.021895405 | | 133 | K133E | 1.033675821 | 0.026820034 |
| | 136 | D136H | 1.026911627 | 0.018990892 | | 53 | I53T | 1.033686572 | 0.037872951 |
| | 97 | R97S | 1.026916053 | 0.02677854 | | 62 | E62G | 1.033801741 | 0.037873937 |
| | 352 | E352Q | 1.027432429 | 0.03781927 | | 209 | V209A | 1.033808874 | 0.02193165 |
| | 56 | T56K | 1.027711332 | 0.026783431 | | 31 | D31H | 1.033878421 | 0.037874594 |
| | 368 | H368Y | 1.033952401 | 0.026821727 | | 210 | V210A | 1.043685765 | 0.021981435 |
| | 143 | N143T | 1.033970129 | 0.019022201 | | 113 | E113G | 1.043721339 | 0.026881365 |
| | 56 | T56S | 1.034180204 | 0.019023131 | | 171 | A171E | 1.043997445 | 0.019066448 |
| | 241 | N241H | 1.034286581 | 0.021934065 | | 248 | I248M | 1.044493515 | 0.026886063 |
| | 370 | L370P | 1.034619176 | 0.037880937 | | 319 | R319K | 1.044716432 | 0.017081961 |
| | 301 | M301L | 1.034935739 | 0.014446369 | | 71 | K71E | 1.044884723 | 0.021987457 |
| | 22 | L22M | 1.034994274 | 0.021937642 | | 34 | G34A | 1.044901894 | 0.026888547 |
| | 283 | D283E | 1.035261247 | 0.026829739 | | 16 | K16M | 1.045016823 | 0.012809059 |
| | 195 | F195I | 1.035322495 | 0.037886956 | | 173 | L173R | 1.045406379 | 0.026891614 |
| | 227 | V227F | 1.035524112 | 0.037888681 | | 359 | A359T | 1.045425136 | 0.019072726 |
| | 246 | F246C | 1.035804193 | 0.037891077 | | 63 | L63F | 1.045548681 | 0.019073269 |
| | 336 | R336K | 1.036161542 | 0.026835246 | | 184 | K184Q | 1.045999743 | 0.037977998 |
| | 248 | I248L | 1.036673054 | 0.026838374 | | 30 | L30S | 1.046066317 | 0.026895625 |
| | 207 | V207A | 1.036771781 | 0.021946617 | | 80 | C80Y | 1.046174757 | 0.01907602 |
| | 351 | I351T | 1.037426817 | 0.037904949 | | 260 | N260D | 1.047000975 | 0.026901303 |
| | 258 | I258M | 1.037731625 | 0.026844843 | | 74 | F74Y | 1.047322194 | 0.021999687 |
| | 246 | F246V | 1.037935109 | 0.026846086 | | 71 | K71T | 1.04738321 | 0.026903624 |
| | 16 | K16N | 1.03805924 | 0.026846844 | | 8 | V8I | 1.047502067 | 0.022000589 |
| | 209 | V209M | 1.038067959 | 0.037910426 | | 360 | F360L | 1.047667427 | 0.022001418 |
| | 359 | A359S | 1.038076889 | 0.037910503 | | 194 | I194V | 1.047695346 | 0.019082697 |
| | 249 | Y249N | 1.038128638 | 0.026847268 | | 39 | V39A | 1.047866428 | 0.037993849 |
| | 43 | S43T | 1.038370365 | 0.021954681 | | 240 | Y240S | 1.048137408 | 0.026908203 |
| | 226 | S226L | 1.038507484 | 0.019042256 | | 80 | C80S | 1.048201873 | 0.022004097 |
| | 237 | S237R | 1.038751046 | 0.019043331 | | 229 | E229G | 1.048764261 | 0.019087388 |
| | 144 | P144S | 1.03897515 | 0.026852437 | | 236 | N236I | 1.049107132 | 0.038004375 |
| | 252 | G252A | 1.039243263 | 0.017060163 | | 153 | A153V | 1.049732349 | 0.019091633 |
| | 133 | K133Q | 1.039482528 | 0.019046559 | | 138 | S138T | 1.049750039 | 0.026917984 |
| | 11 | G11A | 1.039972481 | 0.01904872 | 1408 | 110 | K110T | 1.050283494 | 0.022014521 |
| | 51 | K51T | 1.039982432 | 0.026858583 | 1409 | 12 | P12A | 1.051101249 | 0.019097632 |
| | 359 | A359D | 1.040691261 | 0.026862906 | 1410 | 284 | N284T | 1.051541508 | 0.026928839 |
| | 243 | P243T | 1.041123208 | 0.02686554 | 1411 | 73 | I73V | 1.051943475 | 0.022022824 |
| | 337 | S337F | 1.041301956 | 0.021969447 | 1412 | 250 | V250G | 1.05227545 | 0.017111949 |
| | 321 | L321I | 1.041624695 | 0.037940769 | 1413 | 236 | N236S | 1.052927275 | 0.022027741 |
| | 185 | P185S | 1.042107901 | 0.037944886 | 1414 | 3 | A3G | 1.053073789 | 0.038037969 |
| | 32 | Y32C | 1.042507201 | 0.02197551 | 1415 | 133 | K133R | 1.053374941 | 0.022029978 |
| | 185 | P185Q | 1.042761637 | 0.026875522 | 1416 | 354 | V354F | 1.053630418 | 0.022031254 |
| | 189 | S189A | 1.042771185 | 0.017074222 | 1417 | 132 | T132S | 1.053701087 | 0.038043274 |
| | 344 | S344T | 1.042784449 | 0.017074274 | 1418 | 341 | I341V | 1.05372572 | 0.019109119 |
| | 198 | A198T | 1.0430816 | 0.026877471 | 1419 | 203 | G203A | 1.053783515 | 0.022032018 |
| | 306 | R306H | 1.04310179 | 0.019062507 | 1420 | 72 | V72L | 1.05447237 | 0.019112384 |
| | 261 | N261H | 1.043244488 | 0.026878462 | 1421 | 219 | F219I | 1.054510031 | 0.026946799 |
| 1422 | 354 | V354L | 1.054642656 | 0.022036308 | 1463 | 369 | Q369K | 1.074044893 | 0.022132578 |
| 1423 | 250 | V250A | 1.054880436 | 0.019114168 | 1464 | 2 | D2Y | 1.075804254 | 0.027074662 |
| 1424 | 37 | I37M | 1.055138775 | 0.026950599 | 1465 | 249 | Y249F | 1.076381184 | 0.027078102 |
| 1425 | 359 | A359V | 1.055223226 | 0.038056137 | 1466 | 110 | K110E | 1.076382135 | 0.027078108 |
| 1426 | 94 | K94R | 1.055282793 | 0.017123843 | 1467 | 125 | H125R | 1.076801674 | 0.038237151 |
| 1427 | 93 | L93F | 1.055842136 | 0.038061364 | 1468 | 24 | E24A | 1.076883861 | 0.038237836 |
| 1428 | 73 | I73F | 1.055926966 | 0.022042715 | 1469 | 226 | S226A | 1.077170853 | 0.02708281 |
| 1429 | 193 | V193L | 1.056053886 | 0.017126889 | 1470 | 245 | C245G | 1.078388551 | 0.038250365 |
| 1430 | 349 | V349G | 1.056422531 | 0.038066263 | 1471 | 53 | I53F | 1.079588292 | 0.022159877 |
| 1431 | 138 | S138G | 1.056530075 | 0.019121374 | 1472 | 350 | T350P | 1.08013226 | 0.017221334 |
| 1432 | 74 | F74C | 1.056785099 | 0.026960541 | 1473 | 144 | P144T | 1.080173683 | 0.022162754 |
| 1433 | 344 | S344C | 1.058117276 | 0.022053632 | 1474 | 334 | G334A | 1.080438779 | 0.027102267 |
| 1434 | 94 | K94Q | 1.059108632 | 0.01566953 | 1475 | 97 | R97M | 1.080957628 | 0.022166606 |
| 1435 | 318 | K318Q | 1.059573733 | 0.038092833 | 1476 | 5 | R5S | 1.081995946 | 0.038280355 |
| 1436 | 94 | K94T | 1.060109686 | 0.015673171 | 1477 | 236 | N236K | 1.08233798 | 0.022173384 |
| 1437 | 243 | P243A | 1.060296234 | 0.019137799 | 1478 | 4 | P4T | 1.082960604 | 0.038288364 |
| 1438 | 239 | L239M | 1.060813427 | 0.019140052 | 1479 | 242 | T242S | 1.083266742 | 0.019237192 |
| 1439 | 259 | K259T | 1.061717155 | 0.019143987 | 1480 | 26 | Q26R | 1.083373971 | 0.02711971 |
| 1440 | 42 | M42I | 1.062312763 | 0.026993848 | 1481 | 97 | R97T | 1.083625328 | 0.022179701 |
| 1441 | 221 | L221I | 1.063030343 | 0.026998164 | 1482 | 302 | N302I | 1.083817197 | 0.027122342 |
| 1442 | 356 | K356Q | 1.063567586 | 0.027001393 | 1483 | 148 | Y148F | 1.085213048 | 0.022187484 |
| 1443 | 8 | V8F | 1.063737565 | 0.038127858 | 1484 | 56 | T56I | 1.086282284 | 0.027136963 |
| 1444 | 7 | V7E | 1.063783111 | 0.022081802 | 1485 | 24 | E24K | 1.087487175 | 0.038325877 |
| 1445 | 351 | 1351M | 1.063937009 | 0.038129534 | 1486 | 169 | K169E | 1.08929524 | 0.027154804 |
| 1446 | 255 | L255M | 1.063958047 | 0.03812971 | 1487 | 68 | D68E | 1.093209797 | 0.017272087 |
| 1447 | 72 | V72M | 1.064552932 | 0.022085622 | 1488 | 249 | Y249C | 1.093223295 | 0.027178014 |
| 1448 | 136 | D136E | 1.066657571 | 0.022096057 | 1489 | 17 | L17Q | 1.093279452 | 0.027178346 |
| 1449 | 336 | R336S | 1.06698278 | 0.022097668 | 1490 | 229 | E229A | 1.093639414 | 0.022228669 |
| 1450 | 22 | L22S | 1.06812869 | 0.027028768 | 1491 | 63 | L63V | 1.094298389 | 0.038382124 |
| 1451 | 236 | N236D | 1.06865146 | 0.027031901 | 1492 | 4 | P4R | 1.095322576 | 0.027190396 |
| 1452 | 169 | K169Q | 1.069218894 | 0.019176567 | 1493 | 37 | I37N | 1.09812523 | 0.01930078 |
| 1453 | 146 | A146T | 1.069528098 | 0.019177907 | 1494 | 80 | C80G | 1.099087201 | 0.022255187 |
| 1454 | 15 | A15T | 1.069633752 | 0.019178365 | 1495 | 357 | L357M | 1.099667586 | 0.019307349 |
| 1455 | 325 | L325V | 1.070007588 | 0.038180426 | 1496 | 310 | A310S | 1.100794067 | 0.038435545 |
| 1456 | 37 | I37T | 1.070027558 | 0.015709139 | 1497 | 55 | N55T | 1.101383969 | 0.019314653 |
| 1457 | 240 | Y240N | 1.070374505 | 0.038183495 | 1498 | 144 | P144Q | 1.102107875 | 0.019317731 |
| 1458 | 73 | I73M | 1.070732036 | 0.019183121 | 1499 | 109 | A109T | 1.102363942 | 0.038448424 |
| 1459 | 29 | L29V | 1.07186637 | 0.015715785 | 1500 | 15 | A15S | 1.102453125 | 0.038449155 |
| 1460 | 231 | K231N | 1.072749327 | 0.038203346 | 1501 | 85 | A85G | 1.102518452 | 0.03844969 |
| 1461 | 63 | L63M | 1.073557434 | 0.019195344 | 1502 | 353 | D353E | 1.102883724 | 0.019321029 |
| 1462 | 300 | K300R | 1.073588579 | 0.038210354 | 1503 | 48 | D48G | 1.103352863 | 0.027237615 |
| 1504 | 180 | N180D | 1.104562411 | 0.013756074 | | | | | |
| 1505 | 116 | K116Q | 1.104791943 | 0.022282865 | | | | | |
| 1506 | 121 | I121T | 1.107214876 | 0.03848814 | | | | | |
| 1507 | 46 | S46A | 1.108329901 | 0.017330305 | | | | | |
| 1508 | 253 | L253S | 1.109647998 | 0.027274473 | | | | | |
| 1509 | 31 | D31Y | 1.111463534 | 0.038522828 | | | | | |
| 1510 | 157 | V157L | 1.111850704 | 0.019359039 | | | | | |
| 1511 | 116 | K116T | 1.112918346 | 0.019363552 | | | | | |
| 1512 | 49 | F49Y | 1.115306919 | 0.027307487 | | | | | |
| 1513 | 311 | K311Q | 1.115801754 | 0.038558154 | | | | | |
| 1514 | 157 | V157M | 1.117332793 | 0.019382182 | | | | | |
| 1515 | 144 | P144R | 1.118566065 | 0.027326451 | | | | | |
| 1516 | 221 | L221R | 1.123133874 | 0.027352968 | | | | | |
| 1517 | 31 | D31A | 1.129492825 | 0.038669033 | | | | | |
| 1518 | 95 | A95P | 1.129598314 | 0.038669884 | | | | | |
| 1519 | 110 | K110M | 1.129896896 | 0.038672291 | | | | | |
| 1520 | 73 | I73L | 1.132993647 | 0.038697238 | | | | | |
| 1521 | 356 | K356T | 1.13455638 | 0.027418966 | | | | | |
| 1522 | 19 | A198S | 1.134701443 | 0.019455037 | | | | | |
| 1523 | 80 | C80W | 1.135161692 | 0.038714675 | | | | | |
| 1524 | 55 | N55H | 1.136003362 | 0.038721439 | | | | | |
| 1525 | 106 | A106D | 1.138747567 | 0.019471908 | | | | | |
| 1526 | 121 | I121V | 1.139099785 | 0.022447405 | | | | | |
| 1527 | 117 | K117N | 1.141362413 | 0.038764422 | | | | | |
| 1528 | 37 | I37L | 1.145496529 | 0.015976517 | | | | | |
| 1529 | 117 | K117M | 1.148388604 | 0.015986551 | | | | | |
| 1530 | 117 | K117R | 1.149176254 | 0.015989281 | | | | | |
| 1531 | 174 | V174I | 1.168461287 | 0.038979699 | | | | | |
| 1532 | 117 | K117Q | 1.16958718 | 0.027618667 | | | | | |
| 1533 | 4 | P4H | 1.185679472 | 0.039114721 | | | | | |
| 1534 | 55 | N55K | 1.190639994 | 0.039153373 | | | | | |
| 1535 | 116 | K116E | 1.191674684 | 0.027742554 | | | | | |
| 1536 | 71 | K71Q | 1.228523117 | 0.039444994 | | | | | |
| 1537 | 37 | I37F | 1.257955979 | 0.028105435 | | | | | |

TABLE 2

| SEQ | AA | | Growth | |
|---|---|---|---|---|
| ID NO. | Position | Variant | Estimate | S_Error |
| 1408 | 110 | K110T | 1.050283494 | 0.022014521 |
| 1409 | 12 | P12A | 1.051101249 | 0.019097632 |
| 1410 | 284 | N284T | 1.051541508 | 0.026928839 |
| 1411 | 73 | I73V | 1.051943475 | 0.022022824 |
| 1412 | 250 | V250G | 1.05227545 | 0.017111949 |
| 1413 | 236 | N236S | 1.052927275 | 0.022027741 |
| 1414 | 3 | A3G | 1.053073789 | 0.038037969 |
| 1415 | 133 | K133R | 1.053374941 | 0.022029978 |
| 1416 | 354 | V354F | 1.053630418 | 0.022031254 |
| 1417 | 132 | T132S | 1.053701087 | 0.038043274 |
| 1418 | 341 | I341V | 1.05372572 | 0.019109119 |
| 1419 | 203 | G203A | 1.053783515 | 0.022032018 |
| 1420 | 72 | V72I | 1.05447237 | 0.019112384 |
| 1421 | 219 | F219I | 1.054510031 | 0.026946799 |
| 1422 | 354 | V354L | 1.054642656 | 0.022036308 |
| 1423 | 250 | V250A | 1.054880436 | 0.019114168 |
| 1424 | 37 | I37M | 1.055138775 | 0.026950599 |
| 1425 | 359 | A359V | 1.055223226 | 0.038056137 |
| 1426 | 94 | K94R | 1.055282793 | 0.017123843 |
| 1427 | 93 | L93F | 1.055842136 | 0.038061364 |
| 1428 | 73 | I73F | 1.055926966 | 0.022042715 |
| 1429 | 193 | V193L | 1.056053886 | 0.017126889 |
| 1430 | 349 | V349G | 1.056422531 | 0.038066263 |
| 1431 | 138 | S138G | 1.056530075 | 0.019121374 |
| 1432 | 74 | F74C | 1.056785099 | 0.026960541 |
| 1433 | 344 | S344C | 1.058117276 | 0.022053632 |
| 1434 | 94 | K94Q | 1.059108632 | 0.01566953 |
| 1435 | 318 | K318Q | 1.059573733 | 0.038092833 |
| 1436 | 94 | K94T | 1.060109686 | 0.015673171 |
| 1437 | 243 | P243A | 1.060296234 | 0.019137799 |
| 1438 | 239 | L239M | 1.060813427 | 0.019140052 |
| 1439 | 259 | K259T | 1.061717155 | 0.019143987 |
| 1440 | 42 | M42I | 1.062312763 | 0.026993848 |
| 1441 | 221 | L22I | 1.063030343 | 0.026998164 |
| 1442 | 356 | K356Q | 1.063567586 | 0.027001393 |
| 1443 | 8 | V8F | 1.063737565 | 0.038127858 |
| 1444 | 7 | V7E | 1.063783111 | 0.022081802 |
| 1445 | 351 | I351M | 1.063937009 | 0.038129534 |
| 1446 | 255 | L255M | 1.063958047 | 0.03812971 |
| 1447 | 72 | V72M | 1.064552932 | 0.022085622 |
| 1448 | 136 | D136E | 1.066657571 | 0.022096057 |
| 1449 | 336 | R336S | 1.06698278 | 0.022097668 |
| 1450 | 22 | L22S | 1.06812869 | 0.027028768 |
| 1451 | 236 | N236D | 1.06865146 | 0.027031901 |
| 1452 | 169 | K169Q | 1.069218894 | 0.019176567 |
| 1453 | 146 | A146T | 1.069528098 | 0.019177907 |
| 1454 | 15 | A15T | 1.069633752 | 0.019178365 |
| 1455 | 325 | L325V | 1.070007588 | 0.038180426 |
| 1456 | 37 | I37T | 1.070027558 | 0.015709139 |
| 1457 | 240 | Y240N | 1.070374505 | 0.038183495 |
| 1458 | 73 | I73M | 1.070732036 | 0.019183121 |
| 1459 | 29 | L29V | 1.07186637 | 0.015715785 |
| 1460 | 231 | K231N | 1.072749327 | 0.038203346 |
| 1461 | 63 | L63M | 1.073557434 | 0.019195344 |
| 1462 | 300 | K300R | 1.073588579 | 0.038210354 |
| 1463 | 369 | Q369K | 1.074044893 | 0.022132578 |
| 1464 | 2 | D2Y | 1.075804254 | 0.027074662 |
| 1465 | 249 | Y249F | 1.076381184 | 0.027078102 |
| 1466 | 110 | K110E | 1.076382135 | 0.027078108 |
| 1467 | 125 | H125R | 1.076801674 | 0.038237151 |
| 1468 | 24 | E24A | 1.076883861 | 0.038237836 |
| 1469 | 226 | S226A | 1.077170853 | 0.02708281 |
| 1470 | 245 | C245G | 1.078388551 | 0.038250365 |
| 1471 | 53 | I53F | 1.079588292 | 0.022159877 |
| 1472 | 350 | T350P | 1.08013226 | 0.017221334 |
| 1473 | 144 | P144T | 1.080173683 | 0.022162754 |
| 1474 | 334 | G334A | 1.080438779 | 0.027102267 |
| 1475 | 97 | R97M | 1.080957628 | 0.022166606 |
| 1476 | 5 | R5S | 1.081995946 | 0.038280355 |
| 1477 | 236 | N236K | 1.08233798 | 0.022173384 |
| 1478 | 4 | P4T | 1.082960604 | 0.038288364 |
| 1479 | 242 | T2425 | 1.083266742 | 0.019237192 |
| 1480 | 26 | Q26R | 1.083373971 | 0.02711971 |
| 1481 | 97 | R97T | 1.083625328 | 0.022179701 |
| 1482 | 302 | N302I | 1.083817197 | 0.027122342 |
| 1483 | 148 | Y148F | 1.085213048 | 0.022187484 |
| 1484 | 56 | T56 | 1.086282284 | 0.027136963 |
| 1485 | 24 | E24K | 1.087487175 | 0.038325877 |
| 1486 | 169 | K169E | 1.08929524 | 0.027154804 |
| 1487 | 68 | D68E | 1.093209797 | 0.017272087 |
| 1488 | 249 | Y249C | 1.093223295 | 0.027178014 |
| 1489 | 17 | L17Q | 1.093279452 | 0.027178346 |
| 1490 | 229 | E229A | 1.093639414 | 0.022228669 |
| 1491 | 63 | L63V | 1.094298389 | 0.038382124 |
| 1492 | 4 | P4R | 1.095322576 | 0.027190396 |
| 1493 | 37 | I37N | 1.09812523 | 0.01930078 |
| 1494 | 80 | C80G | 1.099087201 | 0.022255187 |
| 1495 | 357 | L357M | 1.099667586 | 0.019307349 |
| 1496 | 310 | A310S | 1.100794067 | 0.038435545 |
| 1497 | 55 | N55T | 1.101383969 | 0.019314653 |
| 1498 | 144 | P144Q | 1.102107875 | 0.019317731 |
| 1499 | 109 | A109T | 1.102363942 | 0.038448424 |
| 1500 | 15 | A155 | 1.102453125 | 0.038449155 |
| 1501 | 85 | A85G | 1.102518452 | 0.03844969 |
| 1502 | 353 | D353E | 1.102883724 | 0.019321029 |
| 1503 | 48 | D48G | 1.103352863 | 0.027237615 |
| 1504 | 180 | N180D | 1.104562411 | 0.013756074 |
| 1505 | 116 | K116Q | 1.104791943 | 0.022282865 |
| 1506 | 121 | I121T | 1.107214876 | 0.03848814 |
| 1507 | 46 | S46A | 1.108329901 | 0.017330305 |
| 1508 | 253 | L253S | 1.109647998 | 0.027274473 |
| 1509 | 31 | D31Y | 1.111463534 | 0.038522828 |
| 1510 | 157 | V157L | 1.111850704 | 0.019359039 |
| 1511 | 116 | K116T | 1.112918346 | 0.019363552 |
| 1512 | 49 | F49Y | 1.115306919 | 0.027307487 |
| 1513 | 311 | K311Q | 1.115801754 | 0.038558154 |
| 1514 | 157 | V157M | 1.117332793 | 0.019382182 |
| 1515 | 144 | P144R | 1.118566065 | 0.027326451 |
| 1516 | 221 | L221R | 1.123133874 | 0.027352968 |
| 1517 | 31 | D31A | 1.129492825 | 0.038669033 |
| 1518 | 95 | A95P | 1.129598314 | 0.038669884 |
| 1519 | 110 | K110M | 1.129896896 | 0.038672291 |
| 1520 | 73 | I73L | 1.132993647 | 0.038697238 |
| 1521 | 356 | K356T | 1.13455638 | 0.027418966 |
| 1522 | 198 | A198S | 1.134701443 | 0.019455037 |
| 1523 | 80 | C80W | 1.135161692 | 0.038714675 |
| 1524 | 55 | NS5H | 1.136003362 | 0.038721439 |
| 1525 | 106 | A106D | 1.138747567 | 0.019471908 |
| 1526 | 121 | I121V | 1.139099785 | 0.022447405 |
| 1527 | 117 | K117N | 1.141362413 | 0.038764422 |
| 1528 | 37 | I37L | 1.145496529 | 0.015976517 |
| 1529 | 117 | K117M | 1.148388604 | 0.015986551 |
| 1530 | 117 | K117R | 1.149176254 | 0.015989281 |
| 1531 | 174 | V174I | 1.168461287 | 0.038979699 |
| 1532 | 117 | K117Q | 1.16958718 | 0.027618667 |
| 1533 | 4 | P4H | 1.185679472 | 0.039114721 |
| 1534 | 55 | N55K | 1.190639994 | 0.039153373 |
| 1535 | 116 | K116E | 1.191674684 | 0.027742554 |
| 1536 | 71 | K71Q | 1.228523117 | 0.039444994 |
| 1537 | 37 | I37F | 1.257955979 | 0.028105435 |

TABLE 3

| SEQ | AA | | Growth | |
|---|---|---|---|---|
| ID NO. | Position | Variant | Estimate | S_Error |
| 4 | 245 | C245R | −0.082328909 | 0.002800309 |
| 5 | 242 | T242M | −0.080047746 | 0.004149365 |
| 6 | 206 | G206R | −0.078593761 | 0.004638939 |
| 7 | 89 | N89Y | −0.076024013 | 0.010489297 |
| 8 | 119 | G119E | −0.074937864 | 0.010896115 |
| 9 | 13 | G13R | −0.074853551 | 0.007726466 |
| 10 | 40 | L40R | −0.070063395 | 0.005510421 |
| 11 | 25 | I25K | −0.068982638 | 0.007261152 |
| 12 | 78 | G78V | −0.067526096 | 0.009120766 |
| 13 | 63 | L63S | −0.065644994 | 0.013282044 |
| 14 | 78 | G78D | −0.064776836 | 0.009510026 |
| 15 | 148 | Y148N | −0.063441146 | 0.00968306 |
| 16 | 79 | G79R | −0.06339475 | 0.005594851 |
| 17 | 147 | S147Y | −0.062922941 | 0.007959346 |
| 18 | 25 | I25R | −0.060336391 | 0.014216401 |
| 19 | 12 | P12L | −0.058712175 | 0.010231123 |
| 20 | 203 | G203R | −0.055514227 | 0.00862007 |
| 21 | 121 | I121K | −0.052304855 | 0.015352926 |
| 22 | 78 | G78A | −0.051541489 | 0.010924077 |
| 23 | 39 | V39D | −0.051222679 | 0.006927088 |
| 24 | 44 | H44Y | −0.049616696 | 0.01108933 |
| 25 | 152 | C152F | −0.048233654 | 0.011203662 |
| 26 | 156 | T156M | −0.047889982 | 0.009170677 |
| 27 | 179 | S179L | −0.047252936 | 0.011282662 |
| 28 | 155 | E155Q | −0.044703109 | 0.00489603 |
| 29 | 84 | S84R | −0.044655405 | 0.009376991 |
| 30 | 11 | G11R | −0.043414428 | 0.006684315 |
| 31 | 176 | D176H | −0.042784012 | 0.011623159 |
| 32 | 79 | G79W | −0.040856332 | 0.005015548 |
| 33 | 99 | A99D | −0.039340649 | 0.009689034 |
| 34 | 78 | G78R | −0.037862237 | 0.016922998 |
| 35 | 152 | C152R | −0.037857259 | 0.011966819 |
| 36 | 119 | G119R | −0.036858248 | 0.017017083 |
| 37 | 176 | D176A | −0.036050332 | 0.006977958 |
| 38 | 105 | G105V | −0.035764596 | 0.009883154 |
| 39 | 200 | K200N | −0.035476397 | 0.012122799 |
| 40 | 151 | Y151D | −0.035371981 | 0.006065061 |
| 41 | 176 | D176Y | −0.034300397 | 0.012197673 |
| 42 | 108 | S108P | −0.033937177 | 0.012220522 |
| 43 | 197 | G197D | −0.033040395 | 0.012276394 |
| 44 | 82 | Q82R | −0.032856792 | 0.007771615 |
| 45 | 45 | R45S | −0.032410529 | 0.008708271 |
| 46 | 78 | G78C | −0.032387435 | 0.017418183 |
| 47 | 111 | A111P | −0.03188857 | 0.008730803 |
| 48 | 86 | V86D | −0.031614093 | 0.01748482 |
| 49 | 154 | N154Y | −0.031591026 | 0.006182799 |
| 50 | 13 | G13S | −0.031082139 | 0.008765275 |
| 51 | 199 | Q199P | −0.031025408 | 0.012399223 |
| 52 | 148 | Y148H | −0.030834835 | 0.012410653 |
| 53 | 342 | R342Q | −0.030718572 | 0.008780684 |
| 54 | 159 | G159R | −0.030177022 | 0.012449865 |
| 55 | 200 | K200Q | −0.028624921 | 0.012540944 |
| 56 | 148 | Y148D | −0.028175623 | 0.017772252 |
| 57 | 200 | K200T | −0.02738975 | 0.017836024 |
| 58 | 155 | E155V | −0.027151588 | 0.005951959 |
| 59 | 115 | A115D | −0.026906113 | 0.008937588 |
| 60 | 182 | L182R | −0.026329136 | 0.012672131 |
| 61 | 111 | A111D | −0.026209767 | 0.008965372 |
| 62 | 101 | Y101D | −0.02595513 | 0.017950709 |
| 63 | 79 | G79E | −0.024949452 | 0.006010122 |
| 64 | 200 | K200R | −0.024920487 | 0.012750651 |
| 65 | 197 | G197R | −0.022771772 | 0.010506467 |
| 66 | 152 | C152Y | −0.021190719 | 0.012951831 |
| 67 | 176 | D176N | −0.020624056 | 0.00821034 |
| 68 | 155 | E155K | −0.020189244 | 0.006502237 |
| 69 | 198 | A198D | −0.019755153 | 0.013026825 |
| 70 | 200 | K200M | −0.019697931 | 0.006514983 |
| 71 | 97 | R97G | −0.019513188 | 0.018440369 |
| 72 | 188 | V188D | −0.017887665 | 0.013122486 |
| 73 | 14 | P14T | −0.016453347 | 0.018659901 |
| 74 | 12 | P12R | −0.015713772 | 0.010803303 |
| 75 | 89 | N89K | −0.015207742 | 0.010823666 |
| 76 | 38 | S38R | −0.015090776 | 0.010828357 |
| 77 | 101 | Y101N | −0.015080221 | 0.009378011 |
| 78 | 87 | P87R | −0.014097394 | 0.010867951 |
| 79 | 13 | G13C | −0.01406565 | 0.007685717 |
| 80 | 255 | L255P | −0.013439858 | 0.010893923 |
| 81 | 104 | T104P | −0.012901704 | 0.013368129 |
| 82 | 154 | N154D | −0.010154661 | 0.013497883 |
| 83 | 109 | A109P | −0.008011609 | 0.01922845 |
| 84 | 90 | L90P | −0.007327339 | 0.019272381 |
| 85 | 82 | Q82P | −0.006096347 | 0.013682989 |
| 86 | 302 | N302Y | −0.004936069 | 0.013734555 |
| 87 | 14 | P14L | −0.004270715 | 0.009732525 |
| 88 | 14 | P14H | −0.003079532 | 0.011280618 |
| 89 | 199 | Q199K | −0.002457549 | 0.013842805 |
| 90 | 156 | T156P | −0.002365385 | 0.005652926 |
| 91 | 156 | T156R | −0.001642863 | 0.008012374 |
| 92 | 224 | C224W | −0.001438268 | 0.01963861 |
| 93 | 61 | R61P | −0.000806458 | 0.019676705 |
| 94 | 178 | S178P | −0.000281397 | 0.009854098 |
| 95 | 152 | C152W | 0.001299128 | 0.008084169 |
| 96 | 112 | A112P | 0.002333031 | 0.011467791 |
| 97 | 242 | T242P | 0.003618397 | 0.014097941 |
| 98 | 357 | 1357R | 0.004774341 | 0.014144944 |
| 99 | 90 | L90R | 0.00510769 | 0.014158412 |
| 100 | 335 | H335D | 0.007289441 | 0.014245627 |
| 101 | 302 | N302K | 0.009110789 | 0.010123817 |
| 102 | 107 | W107R | 0.009413555 | 0.009062484 |
| 103 | 345 | L345R | 0.009711734 | 0.009069816 |
| 104 | 289 | Y289D | 0.010992319 | 0.010175338 |
| 105 | 108 | S108L | 0.011087689 | 0.020355821 |
| 106 | 148 | Y148S | 0.011829232 | 0.011775635 |
| 107 | 101 | Y101S | 0.011985856 | 0.014428114 |
| 108 | 200 | K200E | 0.01201013 | 0.020405729 |
| 109 | 255 | L255R | 0.013416034 | 0.020481098 |
| 110 | 174 | V174D | 0.014070524 | 0.014506948 |
| 111 | 78 | G78S | 0.014348858 | 0.009181622 |
| 112 | 251 | M251R | 0.014371901 | 0.014518239 |
| 113 | 201 | N201Y | 0.016482842 | 0.011918087 |
| 114 | 147 | S147F | 0.017402604 | 0.011945647 |
| 115 | 107 | W107C | 0.017591838 | 0.010350139 |
| 116 | 203 | G203D | 0.020064779 | 0.020826725 |
| 117 | 242 | T242R | 0.020792734 | 0.020863538 |
| 118 | 102 | V102E | 0.022084658 | 0.011463014 |
| 119 | 299 | S299R | 0.022588407 | 0.014816413 |
| 120 | 199 | Q199L | 0.023921015 | 0.012135712 |
| 121 | 203 | G203V | 0.024015376 | 0.012138399 |
| 122 | 242 | T242K | 0.024221862 | 0.014873595 |
| 123 | 344 | S344Y | 0.026520322 | 0.014952959 |
| 124 | 213 | R213P | 0.026737935 | 0.014960408 |
| 125 | 176 | D176E | 0.027877022 | 0.012246853 |
| 126 | 299 | S299I | 0.032739222 | 0.01516159 |
| 127 | 342 | R342W | 0.032945749 | 0.015168373 |
| 128 | 289 | Y289S | 0.032981137 | 0.02145286 |
| 129 | 174 | V174F | 0.035960653 | 0.010795071 |
| 130 | 107 | W107L | 0.035961132 | 0.021589805 |
| 131 | 147 | S147P | 0.03608325 | 0.009657955 |
| 132 | 332 | L332W | 0.036107153 | 0.00881696 |
| 133 | 344 | S344F | 0.03617341 | 0.012470594 |
| 134 | 361 | M361R | 0.038060211 | 0.015333512 |
| 135 | 70 | Y70D | 0.03831843 | 0.021696318 |
| 136 | 342 | R342P | 0.040753423 | 0.012589137 |
| 137 | 306 | R306P | 0.04292549 | 0.015485809 |
| 138 | 278 | I278N | 0.044085481 | 0.012673326 |
| 139 | 150 | Y150N | 0.044834721 | 0.015544367 |
| 140 | 243 | P243R | 0.045547102 | 0.015566048 |
| 141 | 291 | C291F | 0.049659574 | 0.022188048 |
| 142 | 342 | R342L | 0.056043592 | 0.010040929 |
| 143 | 212 | V212D | 0.058345144 | 0.022543683 |
| 144 | 105 | G105A | 0.058922696 | 0.010092704 |
| 145 | 156 | T156A | 0.062605347 | 0.016060154 |
| 146 | 158 | H158P | 0.068873742 | 0.011477323 |
| 147 | 107 | W107S | 0.069223214 | 0.016240158 |
| 148 | 148 | Y148C | 0.071870983 | 0.023066129 |
| 149 | 361 | M361K | 0.072574696 | 0.013332906 |
| 150 | 45 | R45M | 0.073107727 | 0.009436446 |
| 151 | 157 | V157E | 0.073138611 | 0.008172935 |
| 152 | 175 | C175W | 0.076466775 | 0.016430571 |
| 153 | 289 | Y289N | 0.079028399 | 0.023328835 |
| 154 | 342 | R342G | 0.081698174 | 0.023424595 |
| 155 | 193 | V193E | 0.088728541 | 0.016738611 |
| 156 | 346 | Y346N | 0.092947628 | 0.013750862 |
| 157 | 340 | G340R | 0.096645899 | 0.008466079 |
| 158 | 99 | A99P | 0.099456424 | 0.016994866 |
| 159 | 44 | H44L | 0.100716905 | 0.02407505 |
| 160 | 155 | E155G | 0.109845778 | 0.006762159 |
| 161 | 202 | V202D | 0.112330654 | 0.010935209 |
| 162 | 38 | S38I | 0.118686302 | 0.017427243 |
| 163 | 194 | I194N | 0.128245208 | 0.01763076 |
| 164 | 70 | Y70N | 0.135126482 | 0.010263642 |
| 165 | 178 | S178F | 0.137530207 | 0.017821947 |
| 166 | 102 | V102G | 0.139296116 | 0.025252958 |
| 167 | 178 | S178Y | 0.158569473 | 0.012895235 |
| 168 | 45 | R45G | 0.16656049 | 0.013001076 |
| 169 | 72 | V72G | 0.170212399 | 0.02609055 |
| 170 | 89 | N89I | 0.173467933 | 0.026174203 |
| 171 | 101 | Y101C | 0.176154215 | 0.026242631 |
| 172 | 289 | Y289H | 0.179144051 | 0.011774207 |
| 173 | 45 | R45K | 0.180217905 | 0.010760393 |
| 174 | 241 | N24I | 0.182017486 | 0.026390163 |
| 175 | 45 | R45T | 0.185541241 | 0.010814765 |
| 176 | 70 | Y70S | 0.188310548 | 0.015329318 |
| 177 | 346 | Y346S | 0.190074902 | 0.01880316 |
| 178 | 91 | I91S | 0.201032961 | 0.013430718 |
| 179 | 103 | V103G | 0.206543637 | 0.015581727 |
| 180 | 10 | F10S | 0.20866322 | 0.012094563 |
| 181 | 334 | G334W | 0.214506644 | 0.01921172 |
| 182 | 335 | H335L | 0.215599006 | 0.027191048 |
| 183 | 213 | R213G | 0.220196307 | 0.015763085 |
| 184 | 278 | I278S | 0.224805082 | 0.015822934 |
| 185 | 175 | C175R | 0.227952575 | 0.01229114 |
| 186 | 149 | V149E | 0.23029841 | 0.011243055 |
| 187 | 151 | Y151N | 0.230876574 | 0.011248283 |
| 188 | 89 | N89D | 0.240825557 | 0.016025928 |
| 189 | 282 | I282N | 0.253551504 | 0.01401625 |
| 190 | 302 | N302H | 0.253834708 | 0.014019218 |
| 191 | 291 | C291R | 0.257636655 | 0.016231069 |
| 192 | 199 | Q199E | 0.277049306 | 0.012753757 |
| 193 | 179 | S179P | 0.277668503 | 0.010092833 |
| 194 | 340 | G340D | 0.277723948 | 0.01646656 |
| 195 | 307 | I307N | 0.279333417 | 0.020186058 |
| 196 | 115 | A115T | 0.294683232 | 0.020397449 |
| 197 | 91 | I91T | 0.294786295 | 0.014430187 |
| 198 | 243 | P243H | 0.301863636 | 0.01673703 |
| 199 | 72 | V72E | 0.305608394 | 0.029047232 |
| 200 | 183 | S183P | 0.306386428 | 0.014540589 |
| 201 | 149 | V149G | 0.321080268 | 0.01467701 |
| 202 | 332 | L332S | 0.321549763 | 0.029341476 |
| 203 | 285 | S285Y | 0.328555414 | 0.012045231 |
| 204 | 76 | Q76R | 0.332833444 | 0.020896458 |
| 205 | 353 | D353V | 0.340437769 | 0.029679104 |
| 206 | 305 | F305S | 0.342092143 | 0.017161098 |
| 207 | 198 | A198P | 0.342572751 | 0.029716556 |
| 208 | 99 | A99T | 0.347366056 | 0.029800131 |
| 209 | 324 | A324P | 0.352421138 | 0.017265164 |
| 210 | 44 | H44N | 0.356237655 | 0.02118596 |
| 211 | 244 | P244R | 0.361646356 | 0.030045068 |
| 212 | 272 | S272P | 0.364523652 | 0.021285709 |
| 213 | 329 | M329R | 0.364882391 | 0.021289997 |
| 214 | 155 | E155A | 0.371410186 | 0.008749598 |
| 215 | 44 | H44P | 0.37484773 | 0.017484995 |
| 216 | 11 | G11V | 0.383632995 | 0.017568947 |
| 217 | 346 | Y346C | 0.387170111 | 0.01524906 |
| 218 | 64 | L64P | 0.387670411 | 0.017607139 |
| 219 | 174 | V174G | 0.389703773 | 0.015269783 |
| 220 | 119 | G119V | 0.393854873 | 0.03057669 |
| 221 | 175 | C175F | 0.398035886 | 0.017704097 |
| 222 | 105 | G105E | 0.398082638 | 0.017704531 |
| 223 | 154 | N154I | 0.399043262 | 0.01086556 |
| 224 | 156 | T156S | 0.401749753 | 0.012555852 |
| 225 | 275 | S275P | 0.408073807 | 0.030802885 |
| 226 | 270 | L270P | 0.409424281 | 0.017808868 |
| 227 | 111 | A111V | 0.409640383 | 0.030827506 |
| 228 | 340 | G340C | 0.411727801 | 0.021829242 |
| 229 | 181 | F181S | 0.417778394 | 0.017884596 |
| 230 | 247 | S247R | 0.419320759 | 0.021913041 |
| 231 | 335 | H335P | 0.429131492 | 0.031129066 |
| 232 | 155 | E155D | 0.438346608 | 0.009065438 |
| 233 | 44 | H44Q | 0.453193052 | 0.015764441 |
| 234 | 191 | F191S | 0.456210755 | 0.031534144 |
| 235 | 303 | I303N | 0.456236737 | 0.018221708 |
| 236 | 104 | T104K | 0.457736662 | 0.022323216 |
| 237 | 119 | G119W | 0.461513749 | 0.011209729 |
| 238 | 276 | Q276P | 0.471699457 | 0.014228136 |
| 239 | 14 | P14A | 0.472185444 | 0.012997065 |
| 240 | 59 | L59P | 0.47733623 | 0.015941341 |
| 241 | 122 | N122I | 0.477509059 | 0.022525983 |
| 242 | 17 | L17R | 0.482388406 | 0.031911579 |
| 243 | 115 | A115V | 0.485305372 | 0.015998516 |
| 244 | 14 | P14S | 0.485798131 | 0.031959778 |
| 245 | 11 | G11W | 0.48593221 | 0.018470136 |
| 246 | 299 | S299C | 0.486472656 | 0.022616169 |
| 247 | 22 | L22W | 0.487751499 | 0.018485044 |
| 248 | 364 | F364L | 0.487809309 | 0.022629527 |
| 249 | 45 | R45W | 0.488258523 | 0.011348567 |
| 250 | 241 | N241Y | 0.488607633 | 0.018492048 |
| 251 | 70 | Y70C | 0.491587496 | 0.018516365 |
| 252 | 75 | L75R | 0.492157677 | 0.014359815 |
| 253 | 201 | N201H | 0.492600859 | 0.022677226 |
| 254 | 334 | G334V | 0.494593531 | 0.032083122 |
| 255 | 121 | I121R | 0.496950644 | 0.022720273 |
| 256 | 87 | P87H | 0.497772096 | 0.022728376 |
| 257 | 230 | Y230D | 0.499269433 | 0.022743124 |
| 258 | 124 | V124D | 0.499569422 | 0.022746075 |
| 259 | 216 | L216R | 0.501239281 | 0.032175396 |
| 260 | 187 | D187Y | 0.503123591 | 0.032201416 |
| 261 | 87 | P87L | 0.514909465 | 0.018703571 |
| 262 | 179 | S179A | 0.516164497 | 0.01621458 |
| 263 | 170 | G170V | 0.518491368 | 0.010296482 |
| 264 | 289 | Y289C | 0.518940982 | 0.016233621 |
| 265 | 358 | A358P | 0.519792956 | 0.022942506 |
| 266 | 293 | V293M | 0.51983056 | 0.022942866 |
| 267 | 178 | S178T | 0.520679036 | 0.010306184 |
| 268 | 287 | G287V | 0.526220036 | 0.023003914 |
| 269 | 67 | P67L | 0.529158341 | 0.018815336 |
| 270 | 205 | A205D | 0.529677048 | 0.014592813 |
| 271 | 140 | W140L | 0.534059222 | 0.032619977 |
| 272 | 154 | N154T | 0.540178962 | 0.016377214 |
| 273 | 292 | P292R | 0.540252974 | 0.032701895 |
| 274 | 293 | V293E | 0.54427183 | 0.032754725 |
| 275 | 339 | G339V | 0.54981991 | 0.023225391 |
| 276 | 275 | S275Y | 0.549866486 | 0.018974433 |
| 277 | 361 | M361T | 0.550401774 | 0.032834824 |
| 278 | 150 | Y150C | 0.551441251 | 0.018986375 |
| 279 | 60 | V60E | 0.551672341 | 0.016453453 |
| 280 | 343 | A343D | 0.55656662 | 0.023287587 |
| 281 | 140 | W140R | 0.558596862 | 0.023306209 |
| 282 | 204 | S204Y | 0.559944585 | 0.023318547 |
| 283 | 364 | F3645 | 0.56382133 | 0.023353931 |
| 284 | 193 | V193G | 0.564189298 | 0.019082267 |
| 285 | 83 | F83Y | 0.564884664 | 0.033021808 |
| 286 | 86 | V86G | 0.567130265 | 0.02338401 |
| 287 | 335 | H335R | 0.572706012 | 0.033121499 |
| 288 | 99 | A99V | 0.573484384 | 0.002715355 |
| 289 | 292 | P292Q | 0.573946333 | 0.012569712 |
| 290 | 151 | Y151S | 0.574045196 | 0.016599052 |
| 291 | 321 | L321P | 0.574171502 | 0.01485625 |
| 292 | 213 | R213L | 0.576486177 | 0.033169365 |
| 293 | 211 | I211S | 0.57669275 | 0.023470306 |
| 294 | 89 | N89T | 0.581020561 | 0.013606092 |
| 295 | 161 | E161K | 0.584372326 | 0.019231314 |
| 296 | 320 | F320C | 0.589637969 | 0.033334326 |
| 297 | 180 | N180K | 0.591694722 | 0.013661912 |
| 298 | 147 | S147T | 0.592100995 | 0.023607443 |
| 299 | 10 | F10I | 0.597684085 | 0.033434071 |
| 300 | 263 | G263V | 0.600518275 | 0.023681391 |
| 301 | 292 | P292A | 0.602082139 | 0.023695056 |
| 302 | 245 | C245Y | 0.605150307 | 0.033525847 |
| 303 | 282 | I282S | 0.606074588 | 0.016801412 |
| 304 | 166 | P166L | 0.610121015 | 0.023764942 |
| 305 | 364 | F364C | 0.611007361 | 0.019423077 |
| 306 | 288 | F288C | 0.611267526 | 0.016833582 |
| 307 | 340 | G340S | 0.612312728 | 0.033613197 |
| 308 | 166 | P166T | 0.612329202 | 0.033613397 |
| 309 | 282 | I282T | 0.618336039 | 0.023835748 |
| 310 | 292 | P292S | 0.619841138 | 0.019485497 |
| 311 | 303 | I303F | 0.620209034 | 0.015115782 |
| 312 | 161 | E161V | 0.621328368 | 0.016895419 |
| 313 | 278 | I278T | 0.621954424 | 0.023866742 |
| 314 | 111 | A111T | 0.622045907 | 0.01381732 |
| 315 | 197 | G197V | 0.625705579 | 0.023898751 |
| 316 | 153 | A153P | 0.626193688 | 0.010748249 |
| 317 | 166 | P166H | 0.627686869 | 0.015156791 |
| 318 | 245 | C245F | 0.628300102 | 0.019544738 |
| 319 | 213 | R213C | 0.630034074 | 0.012017426 |
| 320 | 64 | L64R | 0.633251333 | 0.016967889 |
| 321 | 361 | M361V | 0.63485932 | 0.013881524 |
| 322 | 335 | H335Q | 0.635801303 | 0.019596847 |
| 323 | 104 | T104R | 0.636072704 | 0.016984911 |
| 324 | 335 | H335N | 0.63657811 | 0.016987955 |
| 325 | 344 | S344P | 0.636654343 | 0.023991472 |
| 326 | 285 | S285C | 0.639143078 | 0.017003381 |
| 327 | 166 | P1665 | 0.64045269 | 0.019628962 |
| 328 | 216 | L216P | 0.641337862 | 0.033960492 |
| 329 | 320 | F320I | 0.644613789 | 0.033999037 |
| 330 | 140 | W140G | 0.647513162 | 0.019677425 |
| 331 | 338 | V338E | 0.651119042 | 0.024112399 |
| 332 | 206 | G206V | 0.653275821 | 0.01971673 |
| 333 | 66 | V66F | 0.653736025 | 0.019719859 |
| 334 | 228 | L228P | 0.654724848 | 0.024142272 |
| 335 | 364 | F364V | 0.65537621 | 0.024147656 |
| 336 | 195 | F195S | 0.655457024 | 0.034125712 |
| 337 | 43 | S43R | 0.657871438 | 0.017114848 |
| 338 | 233 | Q233P | 0.658716845 | 0.019753637 |
| 339 | 140 | W140C | 0.659814215 | 0.017126295 |
| 340 | 279 | Y279H | 0.660088477 | 0.024186511 |
| 341 | 188 | V188G | 0.661508965 | 0.019772501 |
| 342 | 61 | R61W | 0.662208385 | 0.017140373 |
| 343 | 298 | R298I | 0.663768155 | 0.024216725 |
| 344 | 146 | A146D | 0.664352516 | 0.019791659 |
| 345 | 283 | D283H | 0.665028788 | 0.019796208 |
| 346 | 63 | L63W | 0.665623043 | 0.03424323 |
| 347 | 303 | I303S | 0.66573582 | 0.034244527 |
| 348 | 42 | M42R | 0.666115487 | 0.017163278 |
| 349 | 224 | C224Y | 0.666948377 | 0.017168149 |
| 350 | 285 | S285T | 0.667525293 | 0.024247464 |
| 351 | 209 | V209E | 0.667793207 | 0.024249652 |
| 352 | 320 | F320L | 0.670015455 | 0.024267776 |
| 353 | 364 | F364I | 0.671593524 | 0.017195249 |
| 354 | 174 | V174L | 0.672152189 | 0.034318084 |
| 355 | 357 | L357Q | 0.673355158 | 0.019851971 |
| 356 | 298 | R298S | 0.676302004 | 0.024318835 |
| 357 | 307 | I307S | 0.676365896 | 0.024319352 |
| 358 | 103 | V103E | 0.676820083 | 0.034371306 |
| 359 | 175 | C175Y | 0.677872048 | 0.019882035 |
| 360 | 317 | E317D | 0.678727807 | 0.024338455 |
| 361 | 298 | R298T | 0.680514243 | 0.019899563 |
| 362 | 106 | A106T | 0.681342877 | 0.01990505 |
| 363 | 341 | I341N | 0.681639773 | 0.013072893 |
| 364 | 216 | L216Q | 0.683314206 | 0.024375425 |
| 365 | 180 | N180Y | 0.683564449 | 0.024377437 |
| 366 | 317 | E317G | 0.683626195 | 0.014118821 |
| 367 | 139 | T139P | 0.683658515 | 0.015454466 |
| 368 | 172 | V172G | 0.687701499 | 0.024410639 |
| 369 | 163 | D163V | 0.688209741 | 0.017291226 |
| 370 | 112 | A112E | 0.688925695 | 0.017295328 |
| 371 | 285 | S285A | 0.690587935 | 0.024433727 |
| 372 | 324 | A324D | 0.694011025 | 0.015507835 |
| 373 | 366 | E366V | 0.694228669 | 0.034567685 |
| 374 | 282 | I282F | 0.695224851 | 0.034578823 |
| 375 | 158 | H158Y | 0.695450359 | 0.024472478 |
| 376 | 181 | F181C | 0.696083062 | 0.034588411 |
| 377 | 279 | Y279D | 0.697792852 | 0.024491084 |
| 378 | 359 | A359P | 0.700390061 | 0.034636409 |
| 379 | 191 | F191V | 0.701501819 | 0.034648766 |
| 380 | 347 | N347Y | 0.702893301 | 0.034664215 |
| 381 | 281 | I281F | 0.703510279 | 0.024536325 |
| 382 | 275 | S275T | 0.70405624 | 0.024540633 |
| 383 | 274 | K274I | 0.704291778 | 0.020055359 |
| 384 | 12 | P127 | 0.707721377 | 0.024569494 |
| 385 | 279 | Y279F | 0.708549836 | 0.017406732 |
| 386 | 303 | I303M | 0.710019895 | 0.024587544 |
| 387 | 92 | G92V | 0.710279528 | 0.02458958 |
| 388 | 292 | P292L | 0.711639178 | 0.01742409 |
| 389 | 166 | P166A | 0.713288276 | 0.017433337 |
| 390 | 213 | R213S | 0.713744947 | 0.024616715 |
| 391 | 293 | V293A | 0.714644013 | 0.020122132 |
| 392 | 177 | M177K | 0.716338593 | 0.034812459 |
| 393 | 186 | V186G | 0.717175546 | 0.034821625 |
| 394 | 298 | R298K | 0.718547914 | 0.034836641 |
| 395 | 163 | D163Y | 0.71942511 | 0.024661006 |
| 396 | 328 | N328I | 0.721745514 | 0.020167578 |
| 397 | 134 | I134L | 0.721799956 | 0.024679456 |
| 398 | 210 | V210E | 0.722043002 | 0.020169475 |
| 399 | 60 | V60G | 0.722799381 | 0.017486402 |
| 400 | 298 | R298G | 0.723175528 | 0.020176694 |
| 401 | 202 | V202G | 0.724570716 | 0.020185578 |
| 402 | 279 | Y279S | 0.725502461 | 0.020191504 |
| 403 | 353 | D353A | 0.727612659 | 0.024724448 |
| 404 | 206 | G206E | 0.728913673 | 0.020213159 |
| 405 | 21 | V21E | 0.729839703 | 0.024741624 |
| 406 | 134 | I134T | 0.730291701 | 0.024745106 |
| 407 | 89 | N89S | 0.730322357 | 0.034964695 |
| 408 | 264 | A264D | 0.73071651 | 0.024748377 |
| 409 | 80 | C80R | 0.731988901 | 0.034982709 |
| 410 | 277 | T277P | 0.733092835 | 0.024766652 |
| 411 | 100 | D100Y | 0.735217632 | 0.024782959 |
| 412 | 49 | F495 | 0.735416883 | 0.024784487 |
| 413 | 233 | Q233R | 0.735808984 | 0.035023898 |
| 414 | 320 | F320Y | 0.738694541 | 0.024809578 |
| 415 | 162 | F162V | 0.738913559 | 0.024811252 |
| 416 | 197 | 61979 | 0.739967887 | 0.035068578 |
| 417 | 262 | G262V | 0.7403841 | 0.024822484 |
| 418 | 230 | Y230S | 0.740967168 | 0.01332954 |
| 419 | 134 | I134N | 0.74098913 | 0.0248271 |
| 420 | 15 | A15D | 0.742038921 | 0.020295877 |
| 421 | 94 | K94I | 0.742741262 | 0.024840456 |
| 422 | 368 | H368R | 0.744358039 | 0.020310394 |
| 423 | 70 | Y70H | 0.744879749 | 0.035121134 |
| 424 | 134 | I134S | 0.745871845 | 0.014406983 |
| 425 | 307 | I307T | 0.746114802 | 0.024866113 |
| 426 | 353 | D353Y | 0.746121063 | 0.024866161 |
| 427 | 358 | A358D | 0.746534315 | 0.024869299 |
| 428 | 114 | E114K | 0.748864882 | 0.035163605 |
| 429 | 290 | V290G | 0.749187027 | 0.024889413 |
| 430 | 160 | V160M | 0.750034045 | 0.020345802 |
| 431 | 160 | V160G | 0.751384499 | 0.02490604 |
| 432 | 118 | F118S | 0.752079224 | 0.02491129 |
| 433 | 286 | Q286P | 0.752727059 | 0.020362541 |
| 434 | 204 | S204P | 0.753725299 | 0.020368736 |
| 435 | 124 | V124G | 0.755844904 | 0.035237632 |
| 436 | 56 | T56R | 0.756071292 | 0.024941397 |
| 437 | 158 | H158R | 0.756077185 | 0.00953039 |
| 438 | 329 | M329I | 0.759132557 | 0.020402202 |
| 439 | 347 | N347D | 0.761200639 | 0.015842653 |
| 440 | 161 | E161D | 0.761574246 | 0.017698265 |
| 441 | 288 | F288S | 0.762405262 | 0.024988952 |
| 442 | 162 | F162I | 0.762597089 | 0.035308812 |
| 443 | 248 | I248S | 0.762644786 | 0.035309313 |
| 444 | 333 | K333N | 0.763280272 | 0.024995501 |
| 445 | 180 | N180H | 0.763633795 | 0.012568876 |
| 446 | 182 | L182Q | 0.764029347 | 0.025001103 |
| 447 | 61 | R61L | 0.76475665 | 0.017715346 |
| 448 | 10 | F10V | 0.766130532 | 0.017722706 |
| 449 | 104 | T104I | 0.766652709 | 0.025020696 |
| 450 | 15 | A15P | 0.768745672 | 0.017736693 |
| 451 | 288 | F288V | 0.771137312 | 0.025054088 |
| 452 | 33 | K33 | 0.772116394 | 0.035408451 |
| 453 | 257 | W257S | 0.77455237 | 0.035433817 |
| 454 | 274 | K274E | 0.775155271 | 0.015909845 |
| 455 | 267 | M267K | 0.775709393 | 0.020503845 |
| 456 | 302 | N302D | 0.776140826 | 0.035450329 |
| 457 | 281 | I281N | 0.776776909 | 0.020510342 |
| 458 | 191 | F191L | 0.777130727 | 0.020512495 |
| 459 | 79 | G79A | 0.777873431 | 0.009950077 |
| 460 | 347 | N347T | 0.778694416 | 0.010349282 |
| 461 | 100 | D100G | 0.778992046 | 0.015928187 |
| 462 | 211 | I211T | 0.779526923 | 0.035485453 |
| 463 | 305 | F305L | 0.780485038 | 0.015935309 |
| 464 | 83 | F83C | 0.780569843 | 0.025123908 |
| 465 | 366 | E366K | 0.780850063 | 0.03549915 |
| 466 | 303 | I303T | 0.781106747 | 0.017802397 |
| 467 | 182 | L182P | 0.781483948 | 0.020538923 |
| 468 | 291 | C291S | 0.783137584 | 0.020548937 |
| 469 | 186 | V186E | 0.786059574 | 0.025164288 |
| 470 | 293 | V293L | 0.786532551 | 0.014587281 |
| 471 | 329 | M329V | 0.786689174 | 0.020570399 |
| 472 | 316 | L316S | 0.787204234 | 0.015967255 |
| 473 | 281 | I281S | 0.787444057 | 0.03556718 |
| 474 | 162 | F162S | 0.787852194 | 0.020577413 |
| 475 | 163 | D163H | 0.788147691 | 0.035574417 |
| 476 | 100 | D100V | 0.788279645 | 0.025180566 |
| 477 | 288 | F288I | 0.790662586 | 0.025198004 |
| 478 | 267 | M267R | 0.790839762 | 0.025199299 |
| 479 | 348 | A348G | 0.791700193 | 0.017858182 |
| 480 | 283 | D283V | 0.791874806 | 0.025206862 |
| 481 | 99 | A99G | 0.793486955 | 0.014617539 |
| 482 | 204 | S204F | 0.794767661 | 0.025227964 |
| 483 | 67 | P67R | 0.79586268 | 0.025235938 |
| 484 | 5 | R5G | 0.79597438 | 0.010006425 |
| 485 | 225 | P225L | 0.79612402 | 0.017881337 |
| 486 | 230 | Y230H | 0.79613856 | 0.035656302 |
| 487 | 17 | L17M | 0.797880745 | 0.025250616 |
| 488 | 124 | V124F | 0.798645343 | 0.02525617 |
| 489 | 162 | F162C | 0.799713632 | 0.025263926 |
| 490 | 102 | V102L | 0.800055252 | 0.035696238 |
| 491 | 134 | I134F | 0.800804023 | 0.025271834 |
| 492 | 278 | 278F | 0.800819945 | 0.035704019 |
| 493 | 77 | G77E | 0.801632029 | 0.017910053 |
| 494 | 21 | V21G | 0.802047875 | 0.017912216 |
| 495 | 160 | V160A | 0.802052887 | 0.025280883 |
| 496 | 209 | V209G | 0.802630833 | 0.016039961 |
| 497 | 288 | F288L | 0.803101783 | 0.017917694 |
| 498 | 360 | F360S | 0.80372736 | 0.025293002 |
| 499 | 224 | C224R | 0.803798424 | 0.025293516 |
| 500 | 181 | F181L | 0.804116855 | 0.025295818 |
| 501 | 230 | Y230C | 0.805466148 | 0.020682837 |
| 502 | 89 | N89H | 0.805484507 | 0.020682947 |
| 503 | 189 | S189F | 0.805828224 | 0.035754862 |
| 504 | 282 | I282M | 0.805892101 | 0.017932177 |
| 505 | 191 | F191C | 0.806900784 | 0.016059931 |
| 506 | 314 | D314A | 0.807452328 | 0.025319901 |
| 507 | 75 | L75V | 0.80749895 | 0.035771776 |
| 508 | 10 | F10C | 0.808408898 | 0.025326795 |
| 509 | 178 | S178C | 0.808431782 | 0.02532696 |
| 510 | 20 | S20L | 0.808877203 | 0.025330169 |
| 511 | 56 | T56P | 0.809996511 | 0.017953422 |
| 512 | 67 | P67A | 0.810026707 | 0.013613016 |
| 513 | 306 | R306G | 0.811462207 | 0.020718385 |
| 514 | 69 | N69I | 0.812554401 | 0.013623109 |
| 515 | 258 | I258T | 0.813079592 | 0.020727945 |
| 516 | 134 | I134M | 0.813906571 | 0.0136285 |
| 517 | 190 | K190E | 0.814285997 | 0.020735067 |
| 518 | 57 | E57K | 0.814292372 | 0.03584031 |
| 519 | 179 | S179T | 0.814598844 | 0.014708407 |
| 520 | 126 | P126A | 0.815044723 | 0.035847877 |
| 521 | 305 | F305Y | 0.815313898 | 0.035850583 |
| 522 | 158 | H158L | 0.815386384 | 0.014711768 |
| 523 | 335 | H335Y | 0.81737908 | 0.035871324 |
| 524 | 341 | I341L | 0.817599076 | 0.017992594 |
| 525 | 305 | F305V | 0.81883355 | 0.03588591 |
| 526 | 317 | E317K | 0.819001229 | 0.025402784 |
| 527 | 277 | T277R | 0.819226427 | 0.035889847 |
| 528 | 83 | F83I | 0.819351144 | 0.016117784 |
| 529 | 87 | P875 | 0.819536661 | 0.01800254 |
| 530 | 244 | P244L | 0.820181069 | 0.020769773 |
| 531 | 83 | F83V | 0.82075613 | 0.016124278 |
| 532 | 49 | F49C | 0.821164901 | 0.013657347 |
| 533 | 187 | D187G | 0.82176799 | 0.018013976 |
| 534 | 283 | D283G | 0.821840633 | 0.018014348 |
| 535 | 316 | L316V | 0.822014333 | 0.035917749 |
| 536 | 4 | P4L | 0.82201664 | 0.025424298 |
| 537 | 100 | D100A | 0.822203307 | 0.003127135 |
| 538 | 190 | K190N | 0.822222655 | 0.035919831 |
| 539 | 15 | A15V | 0.822394417 | 0.025426989 |
| 540 | 356 | K356R | 0.822477821 | 0.035922381 |
| 541 | 220 | A220D | 0.823962181 | 0.020791948 |
| 542 | 280 | E280K | 0.824906862 | 0.025444869 |
| 543 | 108 | S108T | 0.825055283 | 0.016144105 |
| 544 | 32 | Y32D | 0.825133435 | 0.03594889 |
| 545 | 263 | G263R | 0.825348125 | 0.02080006 |
| 546 | 354 | V354G | 0.825491265 | 0.012804608 |
| 547 | 84 | S84T | 0.825604328 | 0.018033591 |
| 548 | 361 | M361L | 0.825703331 | 0.020802137 |
| 549 | 307 | I307F | 0.826347574 | 0.020805904 |
| 550 | 250 | V250F | 0.826691798 | 0.035964419 |
| 551 | 225 | P225H | 0.826909852 | 0.025459099 |
| 552 | 74 | F74S | 0.82971074 | 0.016165504 |
| 553 | 268 | E268V | 0.830124276 | 0.035998555 |
| 554 | 114 | E114A | 0.831077618 | 0.020833501 |
| 555 | 366 | E366A | 0.83170405 | 0.036014234 |
| 556 | 299 | S299T | 0.832264169 | 0.025497024 |
| 557 | 284 | N284I | 0.832672161 | 0.036023832 |
| 558 | 234 | A234P | 0.832803159 | 0.036025131 |
| 559 | 101 | Y101H | 0.833586252 | 0.020848096 |
| 560 | 353 | D353G | 0.833852499 | 0.025508243 |
| 561 | 168 | V168D | 0.834229233 | 0.013708883 |
| 562 | 218 | G218W | 0.834365389 | 0.025511863 |
| 563 | 67 | P67T | 0.834429717 | 0.025512317 |
| 564 | 106 | A106P | 0.83473823 | 0.036044292 |
| 565 | 152 | C152G | 0.834830188 | 0.010527461 |
| 566 | 18 | P18Q | 0.835664278 | 0.025521024 |
| 567 | 194 | I194S | 0.835844478 | 0.025522294 |
| 568 | 98 | C98F | 0.836096153 | 0.02086267 |
| 569 | 68 | D68Y | 0.836307973 | 0.018088014 |
| 570 | 99 | A99S | 0.836879259 | 0.020867211 |
| 571 | 90 | L90F | 0.837293879 | 0.025532504 |
| 572 | 202 | V202A | 0.83743934 | 0.025533528 |
| 573 | 54 | N54I | 0.837779805 | 0.018095463 |
| 574 | 103 | V103M | 0.837866104 | 0.0180959 |
| 575 | 290 | V290L | 0.837995367 | 0.025537441 |
| 576 | 67 | P67Q | 0.83890535 | 0.016207547 |
| 577 | 151 | Y151C | 0.838948099 | 0.01810137 |
| 578 | 49 | F49L | 0.839424329 | 0.02554749 |
| 579 | 58 | N58K | 0.840001402 | 0.036096258 |
| 580 | 230 | Y230N | 0.840559875 | 0.025555467 |
| 581 | 327 | L327P | 0.840881425 | 0.020890378 |
| 582 | 338 | V338G | 0.841236827 | 0.016218162 |
| 583 | 203 | G203C | 0.841510825 | 0.018114309 |
| 584 | 102 | V102A | 0.84153231 | 0.025562293 |
| 585 | 242 | T242A | 0.841726018 | 0.025563652 |
| 586 | 61 | R61Q | 0.841882442 | 0.020896161 |
| 587 | 49 | F49V | 0.842263183 | 0.020898359 |
| 588 | 354 | V354A | 0.842901252 | 0.036124796 |
| 589 | 135 | P135L | 0.843049491 | 0.018122065 |
| 590 | 190 | K190T | 0.843211111 | 0.02090383 |
| 591 | 194 | I194F | 0.84340222 | 0.018123842 |
| 592 | 109 | A109V | 0.843771237 | 0.020907061 |
| 593 | 125 | H125D | 0.845088399 | 0.025587211 |
| 594 | 163 | D163A | 0.846197988 | 0.025594971 |
| 595 | 46 | S46P | 0.846469305 | 0.018139272 |
| 596 | 101 | Y101F | 0.846784659 | 0.025599072 |
| 597 | 337 | S337P | 0.847931855 | 0.025607085 |
| 598 | 51 | K51Q | 0.847948461 | 0.018146701 |
| 599 | 59 | L59V | 0.84850922 | 0.020934334 |
| 600 | 297 | N297T | 0.848653845 | 0.01815024 |
| 601 | 124 | V124A | 0.848758627 | 0.020935767 |
| 602 | 307 | I307M | 0.850379557 | 0.025624158 |
| 603 | 266 | A266P | 0.850416021 | 0.020945282 |
| 604 | 280 | E280V | 0.850640065 | 0.025625973 |
| 605 | 159 | G159A | 0.850732213 | 0.012173361 |
| 606 | 224 | C224F | 0.850883494 | 0.025627669 |
| 607 | 149 | V149A | 0.851154152 | 0.018162772 |
| 608 | 116 | K116R | 0.851649255 | 0.036210491 |
| 609 | 154 | N154H | 0.852606858 | 0.018170043 |
| 610 | 263 | G263D | 0.852848615 | 0.025641347 |
| 611 | 52 | I52M | 0.853085977 | 0.020960585 |
| 612 | 240 | Y240D | 0.854066425 | 0.025649814 |
| 613 | 346 | Y346H | 0.854270189 | 0.018178358 |
| 614 | 352 | E352V | 0.854328575 | 0.036236619 |
| 615 | 229 | E229D | 0.854517326 | 0.036238458 |
| 616 | 181 | F181V | 0.854704042 | 0.018180525 |
| 617 | 86 | V86F | 0.854756167 | 0.036240784 |
| 618 | 197 | G197A | 0.855111376 | 0.013790258 |
| 619 | 187 | D187V | 0.855145822 | 0.020972371 |
| 620 | 106 | A106G | 0.855354532 | 0.036246609 |
| 621 | 54 | N54T | 0.855443489 | 0.025659377 |
| 622 | 162 | F162L | 0.855943441 | 0.025662847 |
| 623 | 181 | F181I | 0.856586264 | 0.018189918 |
| 624 | 333 | K333Q | 0.857006633 | 0.020983001 |
| 625 | 136 | D136A | 0.857215028 | 0.036264706 |
| 626 | 67 | P67S | 0.857305603 | 0.020984708 |
| 627 | 136 | D136G | 0.85762089 | 0.020986507 |
| 628 | 339 | G339E | 0.857726085 | 0.036269672 |
| 629 | 68 | D68A | 0.857932525 | 0.020988285 |
| 630 | 257 | W257C | 0.858064613 | 0.03627296 |
| 631 | 262 | G262A | 0.858409833 | 0.018199007 |
| 632 | 274 | K274R | 0.858868594 | 0.025683121 |
| 633 | 84 | S84I | 0.859477733 | 0.018204323 |
| 634 | 248 | I248N | 0.859609695 | 0.036287959 |
| 635 | 159 | G159C | 0.86016135 | 0.025692066 |
| 636 | 71 | K71N | 0.860383624 | 0.021002256 |
| 637 | 42 | M42V | 0.860972321 | 0.025697673 |
| 638 | 349 | V349D | 0.861224107 | 0.018213009 |
| 639 | 282 | I282L | 0.861680078 | 0.036308028 |
| 640 | 328 | N328Y | 0.862792167 | 0.018220798 |
| 641 | 228 | L228R | 0.863355768 | 0.036324247 |
| 642 | 76 | Q76E | 0.864276325 | 0.02102439 |
| 643 | 212 | V212G | 0.864866899 | 0.036338856 |
| 644 | 84 | S84C | 0.864883857 | 0.018231174 |
| 645 | 222 | R222G | 0.865224023 | 0.021029769 |
| 646 | 326 | E326Q | 0.865359108 | 0.025727943 |
| 647 | 35 | V35F | 0.866049574 | 0.021034451 |
| 648 | 43 | S43I | 0.869210273 | 0.018252585 |
| 649 | 7 | V7G | 0.869448748 | 0.025756072 |
| 650 | 183 | S183T | 0.86978644 | 0.025758391 |
| 651 | 76 | Q76H | 0.870376756 | 0.012969158 |
| 652 | 190 | K190R | 0.871132248 | 0.018262075 |
| 653 | 66 | V66L | 0.871174078 | 0.021063453 |
| 654 | 130 | S130R | 0.871256814 | 0.02576848 |
| 655 | 276 | Q276E | 0.871902963 | 0.021067569 |
| 656 | 305 | F305I | 0.873227938 | 0.025781988 |
| 657 | 40 | L40I | 0.873767025 | 0.025785679 |
| 658 | 287 | G287R | 0.87386167 | 0.025786326 |
| 659 | 104 | T104S | 0.874712927 | 0.025792151 |
| 660 | 343 | A343G | 0.874941354 | 0.018280843 |
| 661 | 167 | D167V | 0.875040102 | 0.016370043 |
| 662 | 235 | G235V | 0.875180064 | 0.036438101 |
| 663 | 220 | A220V | 0.875675721 | 0.025798734 |
| 664 | 254 | V254D | 0.875961387 | 0.021090446 |
| 665 | 77 | G77V | 0.875968326 | 0.021090486 |
| 666 | 157 | V157G | 0.876252523 | 0.018287292 |
| 667 | 213 | R213H | 0.876345749 | 0.01828775 |
| 668 | 239 | L239V | 0.876609635 | 0.036451796 |
| 669 | 238 | S238P | 0.876697446 | 0.021094588 |
| 670 | 187 | D187H | 0.876940209 | 0.018290671 |
| 671 | 302 | N302S | 0.876958243 | 0.01829076 |
| 672 | 275 | S275C | 0.877095086 | 0.021096825 |
| 673 | 339 | G339A | 0.877540758 | 0.025811473 |
| 674 | 217 | L217M | 0.877830694 | 0.036463481 |
| 675 | 196 | A196T | 0.878350463 | 0.025816998 |
| 676 | 220 | A220P | 0.878647527 | 0.036471292 |
| 677 | 208 | T208I | 0.879165554 | 0.02110846 |
| 678 | 100 | D100E | 0.879210014 | 0.02110871 |
| 679 | 247 | S247C | 0.87951351 | 0.021110414 |
| 680 | 264 | A264G | 0.879627645 | 0.036480658 |
| 681 | 62 | E62V | 0.879649854 | 0.03648087 |
| 682 | 161 | E161Q | 0.880407865 | 0.025831021 |
| 683 | 290 | V290D | 0.880502939 | 0.025831669 |
| 684 | 183 | S183C | 0.880580226 | 0.018308532 |
| 685 | 6 | Q6H | 0.880863164 | 0.036492455 |
| 686 | 167 | D167Y | 0.880999784 | 0.025835052 |
| 687 | 19 | H19L | 0.881108501 | 0.021119363 |
| 688 | 165 | I165L | 0.881183844 | 0.01831149 |
| 689 | 299 | S299G | 0.881229389 | 0.018311713 |
| 690 | 20 | S20P | 0.88172317 | 0.025839975 |
| 691 | 32 | Y32F | 0.881922264 | 0.021123924 |
| 692 | 98 | C98W | 0.882537096 | 0.036508419 |
| 693 | 175 | C175S | 0.882711692 | 0.025846699 |
| 694 | 153 | A153T | 0.882876763 | 0.021129271 |
| 695 | 258 | I2585 | 0.883130519 | 0.018321018 |
| 696 | 161 | E161G | 0.883255475 | 0.036515265 |
| 697 | 137 | P137R | 0.883516589 | 0.036517752 |
| 698 | 284 | N284Y | 0.883720687 | 0.018323905 |
| 699 | 216 | L216M | 0.884621419 | 0.018328307 |
| 700 | 187 | D187A | 0.886370571 | 0.036544904 |
| 701 | 343 | A343S | 0.887306319 | 0.036553793 |
| 702 | 279 | Y279N | 0.887586775 | 0.036556456 |
| 703 | 51 | K51R | 0.88765417 | 0.036557096 |
| 704 | 123 | I123F | 0.887663864 | 0.021156032 |
| 705 | 21 | V21A | 0.888110944 | 0.025883335 |
| 706 | 130 | S130T | 0.888184587 | 0.021158937 |
| 707 | 36 | G36C | 0.888522273 | 0.016429597 |
| 708 | 308 | G308D | 0.888813046 | 0.025888088 |
| 709 | 188 | V188F | 0.888939528 | 0.036569293 |
| 710 | 126 | P126R | 0.889451772 | 0.021166003 |
| 711 | 215 | D215V | 0.889875041 | 0.025895273 |
| 712 | 33 | K33R | 0.889905984 | 0.021168534 |
| 713 | 207 | V207F | 0.890491818 | 0.025899443 |
| 714 | 100 | D100H | 0.891500878 | 0.036593564 |
| 715 | 183 | S183Y | 0.892235238 | 0.025911221 |
| 716 | 328 | N328S | 0.892437407 | 0.021182624 |
| 717 | 91 | I91V | 0.892984935 | 0.016449186 |
| 718 | 127 | K127R | 0.893350295 | 0.021187699 |
| 719 | 217 | L217R | 0.893509184 | 0.025919817 |
| 720 | 91 | I91F | 0.893516921 | 0.01393685 |
| 721 | 143 | N143K | 0.893614779 | 0.018372109 |
| 722 | 201 | N201K | 0.89367861 | 0.02592096 |
| 723 | 21 | V21L | 0.893832481 | 0.018373166 |
| 724 | 297 | N297D | 0.893867114 | 0.018373334 |
| 725 | 262 | G262R | 0.89397639 | 0.025922968 |
| 726 | 312 | G312V | 0.894072677 | 0.025923617 |
| 727 | 135 | P135T | 0.894466403 | 0.036621606 |
| 728 | 69 | N69D | 0.894836384 | 0.013941818 |
| 729 | 55 | N55I | 0.894916902 | 0.0211964 |
| 730 | 60 | V60A | 0.895282059 | 0.018380199 |
| 731 | 69 | N69Y | 0.895482182 | 0.036631197 |
| 732 | 250 | V250D | 0.89554547 | 0.036631794 |
| 733 | 165 | I165R | 0.895696873 | 0.036633223 |
| 734 | 300 | K300T | 0.895737893 | 0.03663361 |
| 735 | 201 | N201I | 0.896044029 | 0.018383893 |
| 736 | 323 | K323N | 0.896334237 | 0.016463847 |
| 737 | 53 | I53N | 0.896614501 | 0.025940738 |
| 738 | 20 | S20T | 0.896667072 | 0.025941091 |
| 739 | 276 | Q276L | 0.896914936 | 0.025942759 |
| 740 | 190 | K190Q | 0.897204572 | 0.018389515 |
| 741 | 128 | L128V | 0.897466753 | 0.036649913 |
| 742 | 204 | S204C | 0.897469553 | 0.018390798 |
| 743 | 187 | D187N | 0.897719602 | 0.036652296 |
| 744 | 83 | F83L | 0.897840028 | 0.016470427 |
| 745 | 215 | D215N | 0.898113957 | 0.036656011 |
| 746 | 353 | D353N | 0.898251117 | 0.036657303 |
| 747 | 19 | H19Q | 0.898391863 | 0.021215666 |
| 748 | 119 | G119A | 0.898459909 | 0.021216043 |
| 749 | 57 | E57D | 0.898703355 | 0.025954783 |
| 750 | 334 | G334E | 0.898731966 | 0.016474322 |
| 751 | 265 | A265T | 0.898838036 | 0.01839742 |
| 752 | 32 | Y32H | 0.89916833 | 0.025957907 |
| 753 | 73 | I73S | 0.89957661 | 0.025960649 |
| 754 | 108 | S108A | 0.899759621 | 0.018401877 |
| 755 | 35 | V35I | 0.899801436 | 0.036671896 |
| 756 | 81 | G81S | 0.900004337 | 0.018403059 |
| 757 | 186 | V186M | 0.900061839 | 0.018403337 |
| 758 | 165 | I165K | 0.900395963 | 0.025966149 |
| 759 | 331 | S331T | 0.901205202 | 0.021231228 |
| 760 | 23 | L23I | 0.901208812 | 0.025971602 |
| 761 | 317 | E317Q | 0.901586175 | 0.025974132 |
| 762 | 39 | V39L | 0.901644705 | 0.025974525 |
| 763 | 139 | T139S | 0.901666919 | 0.036689434 |
| 764 | 126 | P126L | 0.901954446 | 0.036692135 |
| 765 | 19 | H19D | 0.902048297 | 0.018412931 |
| 766 | 18 | P18A | 0.902533695 | 0.016490895 |
| 767 | 212 | V212F | 0.902643086 | 0.025981216 |
| 768 | 81 | G81C | 0.903052312 | 0.013085817 |
| 769 | 158 | H158Q | 0.90310801 | 0.013086013 |
| 770 | 173 | L173P | 0.903479 | 0.018419832 |
| 771 | 348 | A348V | 0.903888839 | 0.03671029 |
| 772 | 316 | L316F | 0.903931195 | 0.025989842 |
| 773 | 180 | N180I | 0.904004239 | 0.015077982 |
| 774 | 57 | E57V | 0.904009427 | 0.025990365 |
| 775 | 235 | G235E | 0.90404095 | 0.01842254 |
| 776 | 304 | P304A | 0.904149893 | 0.036712739 |
| 777 | 145 | D145V | 0.904196863 | 0.036713179 |
| 778 | 24 | E24D | 0.904889595 | 0.025996254 |
| 779 | 112 | A112T | 0.904947781 | 0.021251882 |
| 780 | 323 | K323R | 0.905274348 | 0.036723278 |
| 781 | 296 | Q296P | 0.905451542 | 0.026000012 |
| 782 | 43 | S43G | 0.905869765 | 0.036728856 |
| 783 | 351 | I351V | 0.905898301 | 0.021257118 |
| 784 | 69 | N69K | 0.906130689 | 0.018432604 |
| 785 | 334 | G334R | 0.906164748 | 0.021258586 |
| 786 | 222 | R222P | 0.906476953 | 0.021260305 |
| 787 | 120 | T120S | 0.906647547 | 0.021261244 |
| 788 | 28 | E28A | 0.907207327 | 0.026011743 |
| 789 | 366 | E366G | 0.907380923 | 0.018438618 |
| 790 | 168 | V168I | 0.907712289 | 0.036746099 |
| 791 | 72 | V72A | 0.907758218 | 0.026015421 |
| 792 | 353 | D353H | 0.90792929 | 0.021268296 |
| 793 | 341 | I341S | 0.907954809 | 0.036748367 |
| 794 | 93 | L935 | 0.907981141 | 0.018441503 |
| 795 | 142 | L142H | 0.908048482 | 0.026017358 |
| 796 | 139 | T139N | 0.90854292 | 0.016517003 |
| 797 | 31 | D31E | 0.908592941 | 0.026020991 |
| 798 | 137 | P137T | 0.908752087 | 0.026022052 |
| 799 | 181 | F181Y | 0.90884184 | 0.021273313 |
| 800 | 309 | N309D | 0.908957355 | 0.021273947 |
| 801 | 103 | V103A | 0.90899839 | 0.036758122 |
| 802 | 75 | L75P | 0.909117597 | 0.01237244 |
| 803 | 85 | A85V | 0.909176077 | 0.018447243 |
| 804 | 125 | H125P | 0.909224233 | 0.036760232 |
| 805 | 222 | R222L | 0.909226007 | 0.036760248 |
| 806 | 163 | D163E | 0.909250181 | 0.018447599 |
| 807 | 215 | D215H | 0.909272213 | 0.018447705 |
| 808 | 333 | K333E | 0.909374385 | 0.036761634 |
| 809 | 129 | G129W | 0.909510013 | 0.021276984 |
| 810 | 309 | N309I | 0.909525958 | 0.03676305 |
| 811 | 369 | Q369L | 0.909666799 | 0.036764365 |
| 812 | 170 | G170A | 0.909761077 | 0.021278363 |
| 813 | 306 | R306C | 0.909822656 | 0.018450347 |
| 814 | 247 | S247I | 0.910030135 | 0.036767758 |
| 815 | 103 | V103L | 0.910147763 | 0.018451908 |
| 816 | 18 | P18R | 0.910869596 | 0.021284449 |
| 817 | 347 | N347H | 0.910908598 | 0.026036425 |
| 818 | 57 | E57G | 0.911154182 | 0.018456735 |
| 819 | 25 | I25M | 0.911339897 | 0.021287029 |
| 820 | 244 | P244S | 0.91148105 | 0.026040236 |
| 821 | 257 | W257G | 0.911496613 | 0.021287889 |
| 822 | 325 | L325H | 0.911534621 | 0.026040593 |
| 823 | 273 | I2735 | 0.911807008 | 0.026042405 |
| 824 | 123 | I123V | 0.911912593 | 0.015109575 |
| 825 | 300 | K300E | 0.911921021 | 0.036785399 |
| 826 | 53 | I53S | 0.911960508 | 0.026043427 |
| 827 | 82 | Q82L | 0.912252812 | 0.026045372 |
| 828 | 327 | L327H | 0.912490257 | 0.026046951 |
| 829 | 270 | L270R | 0.912744126 | 0.021294729 |
| 830 | 36 | G36V | 0.912770202 | 0.021294872 |
| 831 | 266 | A266V | 0.9131976 | 0.021297214 |
| 832 | 21 | V21M | 0.913295265 | 0.013121873 |
| 833 | 282 | I282V | 0.913568042 | 0.021299243 |
| 834 | 300 | K300M | 0.914228506 | 0.036806894 |
| 835 | 271 | S271G | 0.914262879 | 0.026058735 |
| 836 | 189 | S189P | 0.914328521 | 0.036807825 |
| 837 | 50 | A50G | 0.914416969 | 0.021303892 |
| 838 | 224 | C224S | 0.914429523 | 0.021303961 |
| 839 | 124 | V124L | 0.914608751 | 0.016543248 |
| 840 | 52 | I52L | 0.914699022 | 0.021305436 |
| 841 | 277 | T277A | 0.914782215 | 0.018474111 |
| 842 | 62 | E62Q | 0.915179165 | 0.021308063 |
| 843 | 323 | K323Q | 0.915473325 | 0.036818475 |
| 844 | 313 | D313Y | 0.915477029 | 0.026066797 |
| 845 | 171 | A171S | 0.915905558 | 0.026069641 |
| 846 | 328 | N328K | 0.916090379 | 0.018480365 |
| 847 | 52 | I52T | 0.916214092 | 0.021313723 |
| 848 | 165 | I165M | 0.916457119 | 0.0260733 |
| 849 | 27 | K27Q | 0.916732979 | 0.018483435 |
| 850 | 16 | K16R | 0.9167423 | 0.036830269 |
| 851 | 24 | E24G | 0.916942954 | 0.036832133 |
| 852 | 288 | F288Y | 0.917075716 | 0.021318433 |
| 853 | 255 | L255Q | 0.917111496 | 0.021318628 |
| 854 | 220 | A220T | 0.917242125 | 0.026078505 |
| 855 | 195 | F195C | 0.917337362 | 0.026079136 |
| 856 | 132 | T132R | 0.917985898 | 0.018489418 |
| 857 | 286 | Q286R | 0.918660249 | 0.036848075 |
| 858 | 92 | G92A | 0.918824199 | 0.011782582 |
| 859 | 188 | V188A | 0.918863143 | 0.026089244 |
| 860 | 369 | Q369R | 0.919135294 | 0.026091046 |
| 861 | 125 | H125L | 0.91919313 | 0.036853017 |
| 862 | 256 | E256Q | 0.919378832 | 0.036854739 |
| 863 | 56 | T56A | 0.919486616 | 0.018496576 |
| 864 | 268 | E268K | 0.92000931 | 0.02609683 |
| 865 | 137 | P137Q | 0.920337847 | 0.021336236 |
| 866 | 54 | N54D | 0.920771965 | 0.03686765 |
| 867 | 59 | L59I | 0.92089017 | 0.013148456 |
| 868 | 57 | E57A | 0.920950548 | 0.018503552 |
| 869 | 147 | S147A | 0.921052988 | 0.021340133 |
| 870 | 94 | K94E | 0.921137179 | 0.021340592 |
| 871 | 51 | K51M | 0.921149265 | 0.021340658 |
| 872 | 237 | S237N | 0.921297567 | 0.036872517 |
| 873 | 60 | V60L | 0.921312159 | 0.021341545 |
| 874 | 253 | L253M | 0.921610239 | 0.036875412 |
| 875 | 236 | N236H | 0.921731961 | 0.021343832 |
| 876 | 235 | G235A | 0.921761278 | 0.018507413 |
| 877 | 211 | I211F | 0.9217854 | 0.036877034 |
| 878 | 16 | K16Q | 0.921870589 | 0.021344587 |
| 879 | 65 | A65V | 0.922063631 | 0.026110411 |
| 880 | 283 | D283A | 0.922201438 | 0.036880884 |
| 881 | 163 | D163G | 0.922304842 | 0.015150837 |
| 882 | 287 | G287A | 0.922661653 | 0.021348893 |
| 883 | 225 | P225A | 0.922854716 | 0.021349944 |
| 884 | 127 | K127N | 0.922921698 | 0.036887547 |
| 885 | 33 | K33E | 0.922921968 | 0.02135031 |
| 886 | 364 | F364Y | 0.922933927 | 0.026116159 |
| 887 | 59 | L59H | 0.92310104 | 0.036889205 |
| 888 | 39 | V39F | 0.923231718 | 0.026118124 |
| 889 | 109 | A109G | 0.923541509 | 0.015155728 |
| 890 | 171 | A171V | 0.923877075 | 0.011798635 |
| 891 | 227 | V227D | 0.924077375 | 0.016584 |
| 892 | 277 | T277I | 0.924179306 | 0.036899172 |
| 893 | 217 | L217Q | 0.924451425 | 0.018520206 |
| 894 | 177 | M177T | 0.924654948 | 0.018521173 |
| 895 | 19 | H19P | 0.924685476 | 0.036903848 |
| 896 | 164 | F164S | 0.92479435 | 0.036904853 |
| 897 | 263 | G263C | 0.924797943 | 0.026128456 |
| 898 | 223 | E223V | 0.925343193 | 0.018524442 |
| 899 | 216 | L216V | 0.925515205 | 0.021364408 |
| 900 | 48 | D48N | 0.925515979 | 0.018525263 |
| 901 | 217 | L217V | 0.925627237 | 0.018525791 |
| 902 | 229 | E229Q | 0.925767914 | 0.036913841 |
| 903 | 295 | P295L | 0.925876322 | 0.036914842 |
| 904 | 113 | E113V | 0.92609559 | 0.021367559 |
| 905 | 10 | F10L | 0.926191024 | 0.026137636 |
| 906 | 164 | F164I | 0.926271869 | 0.021368516 |
| 907 | 303 | I303V | 0.926343103 | 0.026138638 |
| 908 | 38 | S38G | 0.926485276 | 0.021369675 |
| 909 | 323 | K323E | 0.926637282 | 0.026140575 |
| 910 | 58 | N58T | 0.927601307 | 0.026146921 |
| 911 | 297 | N297H | 0.927832765 | 0.026148443 |
| 912 | 267 | M267T | 0.927848489 | 0.018536328 |
| 913 | 5 | R5T | 0.927877698 | 0.008890155 |
| 914 | 186 | V186A | 0.928070312 | 0.01853738 |
| 915 | 58 | N58I | 0.928290771 | 0.015174475 |
| 916 | 27 | K27T | 0.928330184 | 0.021379682 |
| 917 | 146 | A146P | 0.928494112 | 0.036938976 |
| 918 | 308 | G308A | 0.928566404 | 0.018539731 |
| 919 | 133 | K133I | 0.928625422 | 0.01854001 |
| 920 | 244 | P244T | 0.928751045 | 0.026154483 |
| 921 | 252 | G252V | 0.928931112 | 0.021382939 |
| 922 | 224 | C224G | 0.929024875 | 0.021383447 |
| 923 | 92 | G92S | 0.929351782 | 0.018543451 |
| 924 | 81 | G81V | 0.929424333 | 0.036947541 |
| 925 | 202 | V202L | 0.929673473 | 0.036949834 |
| 926 | 168 | V168L | 0.929674474 | 0.036949844 |
| 927 | 290 | V290I | 0.929961668 | 0.018546338 |
| 928 | 104 | T104A | 0.930140769 | 0.026163615 |
| 929 | 185 | P185A | 0.930263031 | 0.018547765 |
| 930 | 240 | Y240H | 0.93027981 | 0.026164528 |
| 931 | 28 | E28Q | 0.930345836 | 0.018548156 |
| 932 | 345 | L345M | 0.93041652 | 0.016611138 |
| 933 | 36 | G36R | 0.93050642 | 0.018548916 |
| 934 | 198 | A198V | 0.930616429 | 0.016611992 |
| 935 | 272 | S272T | 0.930643828 | 0.018549566 |
| 936 | 333 | K333I | 0.930678502 | 0.021392402 |
| 937 | 341 | I341M | 0.930807516 | 0.0213931 |
| 938 | 263 | G263S | 0.930811855 | 0.036960306 |
| 939 | 280 | E280G | 0.931448788 | 0.016615547 |
| 940 | 274 | K274T | 0.931625177 | 0.026173359 |
| 941 | 165 | I165T | 0.931972641 | 0.026175639 |
| 942 | 2 | D2A | 0.932017566 | 0.026175933 |
| 943 | 296 | Q296H | 0.932098375 | 0.018556445 |
| 944 | 109 | A109D | 0.932470501 | 0.036975549 |
| 945 | 114 | E114V | 0.932755254 | 0.026180771 |
| 946 | 355 | Q355H | 0.933036933 | 0.011291213 |
| 947 | 318 | K318E | 0.933711258 | 0.018564064 |
| 948 | 254 | V254I | 0.933888784 | 0.013193654 |
| 949 | 322 | D322V | 0.934363214 | 0.03699292 |
| 950 | 153 | A153S | 0.934624663 | 0.021413729 |
| 951 | 98 | C98Y | 0.934748373 | 0.021414396 |
| 952 | 215 | D215Y | 0.934842286 | 0.036997313 |
| 953 | 73 | I73N | 0.93509178 | 0.026196076 |
| 954 | 241 | N241T | 0.935214162 | 0.037000722 |
| 955 | 141 | N141H | 0.935230644 | 0.014091848 |
| 956 | 363 | K363N | 0.93537952 | 0.026197959 |
| 957 | 35 | V35L | 0.935420904 | 0.018572131 |
| 958 | 120 | T120P | 0.935594651 | 0.016633221 |
| 959 | 281 | I281V | 0.935968179 | 0.02620181 |
| 960 | 225 | P225S | 0.936036146 | 0.021421343 |
| 961 | 212 | V212I | 0.936095518 | 0.026202643 |
| 962 | 109 | A109S | 0.936267832 | 0.021422592 |
| 963 | 68 | D68G | 0.936402948 | 0.018576761 |
| 964 | 274 | K274Q | 0.936429731 | 0.026204829 |
| 965 | 221 | L221P | 0.936599174 | 0.021424378 |
| 966 | 215 | D215G | 0.936963807 | 0.02620832 |
| 967 | 30 | L30F | 0.937099176 | 0.018580042 |
| 968 | 24 | E24V | 0.937103721 | 0.015209109 |
| 969 | 64 | L64V | 0.937390097 | 0.02142864 |
| 970 | 100 | D100N | 0.937533666 | 0.026212044 |
| 971 | 177 | M177V | 0.937781592 | 0.018583255 |
| 972 | 128 | L128F | 0.938119935 | 0.02143257 |
| 973 | 92 | G92C | 0.938167122 | 0.01858507 |
| 974 | 160 | V160L | 0.938261917 | 0.037028626 |
| 975 | 198 | A198G | 0.938516966 | 0.021434708 |
| 976 | 28 | E28D | 0.938601129 | 0.016646007 |
| 977 | 87 | P87T | 0.938617343 | 0.018587189 |
| 978 | 147 | S147C | 0.938674845 | 0.021435558 |
| 979 | 268 | E268A | 0.939020417 | 0.018589086 |
| 980 | 5 | R5M | 0.939141343 | 0.016648302 |
| 981 | 290 | V290A | 0.939470057 | 0.037039671 |
| 982 | 85 | A85T | 0.939821425 | 0.018592853 |
| 983 | 225 | P225T | 0.940320162 | 0.037047437 |
| 984 | 247 | S247G | 0.940329202 | 0.021444456 |
| 985 | 319 | R319G | 0.940392386 | 0.037048096 |
| 986 | 19 | H19R | 0.940438815 | 0.026231006 |
| 987 | 365 | L365W | 0.940701163 | 0.021446456 |
| 988 | 307 | I307V | 0.940719946 | 0.021446557 |
| 989 | 142 | L142V | 0.940749094 | 0.021446713 |
| 990 | 223 | E223K | 0.940751387 | 0.021446726 |
| 991 | 128 | L128R | 0.940777072 | 0.018597345 |
| 992 | 183 | S183A | 0.940831946 | 0.03705211 |
| 993 | 132 | T132I | 0.940894023 | 0.026233974 |
| 994 | 257 | W257R | 0.941090214 | 0.026235253 |
| 995 | 312 | G312R | 0.94109781 | 0.037054537 |
| 996 | 135 | P135A | 0.941136199 | 0.021448794 |
| 997 | 313 | D313A | 0.94125279 | 0.026236312 |
| 998 | 276 | Q276K | 0.941308826 | 0.037056462 |
| 999 | 87 | P87A | 0.941487755 | 0.021450682 |
| 1000 | 204 | S204A | 0.941579378 | 0.021451174 |
| 1001 | 38 | S38C | 0.941884947 | 0.037061719 |
| 1002 | 362 | K362R | 0.941962114 | 0.018602911 |
| 1003 | 207 | V207L | 0.942222218 | 0.026242628 |
| 1004 | 250 | V250I | 0.942424459 | 0.021455712 |
| 1005 | 331 | S331Y | 0.942484381 | 0.021456034 |
| 1006 | 139 | T139A | 0.942622671 | 0.021456776 |
| 1007 | 3 | A3D | 0.942676001 | 0.015230905 |
| 1008 | 65 | A65D | 0.942733688 | 0.026245958 |
| 1009 | 294 | E294G | 0.942791904 | 0.015231357 |
| 1010 | 368 | H368P | 0.942863277 | 0.018607141 |
| 1011 | 146 | A146G | 0.942876918 | 0.018607205 |
| 1012 | 237 | S237I | 0.942943031 | 0.018607515 |
| 1013 | 139 | T139I | 0.943222594 | 0.037073915 |
| 1014 | 164 | F164Y | 0.943337152 | 0.02146061 |
| 1015 | 54 | N54Y | 0.943445184 | 0.015233907 |
| 1016 | 184 | K184N | 0.94346195 | 0.01860995 |
| 1017 | 61 | R61G | 0.943616652 | 0.021462109 |
| 1018 | 102 | V102M | 0.943654016 | 0.01861085 |
| 1019 | 60 | V60M | 0.943734177 | 0.018611226 |
| 1020 | 307 | I307L | 0.944200122 | 0.018613411 |
| 1021 | 52 | I52N | 0.944219141 | 0.026255623 |
| 1022 | 363 | K363R | 0.944667181 | 0.016671729 |
| 1023 | 187 | D187E | 0.944831577 | 0.016672425 |
| 1024 | 317 | E317A | 0.944866421 | 0.026259831 |
| 1025 | 149 | V149L | 0.945001703 | 0.013232005 |
| 1026 | 62 | E62A | 0.945091919 | 0.03709094 |
| 1027 | 130 | S130C | 0.945185442 | 0.02147052 |
| 1028 | 265 | A265S | 0.94566961 | 0.037096196 |
| 1029 | 145 | D145E | 0.945833493 | 0.021473991 |
| 1030 | 202 | V202I | 0.945938883 | 0.0262668 |
| 1031 | 122 | N122H | 0.946167925 | 0.01862263 |
| 1032 | 82 | Q82E | 0.946174124 | 0.026268328 |
| 1033 | 64 | L64Q | 0.946266973 | 0.037101629 |
| 1034 | 267 | M267L | 0.94634685 | 0.026269449 |
| 1035 | 59 | L59F | 0.946403889 | 0.037102874 |
| 1036 | 130 | S130G | 0.946418581 | 0.037103008 |
| 1037 | 248 | I248F | 0.946690292 | 0.037105478 |
| 1038 | 145 | D145A | 0.946713328 | 0.016680382 |
| 1039 | 16 | K16E | 0.946954586 | 0.026273395 |
| 1040 | 237 | S237G | 0.947271413 | 0.018627795 |
| 1041 | 167 | D167E | 0.947286494 | 0.021481769 |
| 1042 | 150 | Y150H | 0.947357187 | 0.0132401 |
| 1043 | 286 | Q286L | 0.947396635 | 0.026276264 |
| 1044 | 297 | N297Y | 0.947541982 | 0.026277207 |
| 1045 | 95 | A95E | 0.947543805 | 0.021483146 |
| 1046 | 300 | K300N | 0.94765708 | 0.021483751 |
| 1047 | 247 | S247T | 0.947709201 | 0.018629843 |
| 1048 | 33 | K33Q | 0.947813224 | 0.018630329 |
| 1049 | 123 | I123T | 0.947858614 | 0.018630541 |
| 1050 | 269 | K269E | 0.94794221 | 0.013242108 |
| 1051 | 251 | M251T | 0.948274057 | 0.016686974 |
| 1052 | 153 | A153G | 0.948342508 | 0.026282399 |
| 1053 | 230 | Y230F | 0.948386532 | 0.026282684 |
| 1054 | 135 | P135R | 0.948392119 | 0.026282721 |
| 1055 | 164 | F164V | 0.948450989 | 0.026283102 |
| 1056 | 171 | A171P | 0.948627119 | 0.018634134 |
| 1057 | 145 | D145Y | 0.948704648 | 0.021489353 |
| 1058 | 328 | N328T | 0.948730702 | 0.018634618 |
| 1059 | 270 | L270V | 0.948765843 | 0.037124333 |
| 1060 | 25 | I25V | 0.948807387 | 0.026285413 |
| 1061 | 121 | I121L | 0.94900844 | 0.018635916 |
| 1062 | 189 | S189C | 0.94921066 | 0.026288027 |
| 1063 | 266 | A266S | 0.949279565 | 0.037128995 |
| 1064 | 35 | V35G | 0.949600331 | 0.018638682 |
| 1065 | 98 | C985 | 0.949834791 | 0.037134032 |
| 1066 | 301 | M301T | 0.949963633 | 0.037135201 |
| 1067 | 212 | V212L | 0.949966079 | 0.021496092 |
| 1068 | 7 | V7M | 0.950056922 | 0.037136047 |
| 1069 | 167 | D167N | 0.950080531 | 0.018640925 |
| 1070 | 121 | I121M | 0.950238081 | 0.026294682 |
| 1071 | 350 | T350I | 0.950433003 | 0.021498586 |
| 1072 | 295 | P295T | 0.950448686 | 0.018642643 |
| 1073 | 358 | A358V | 0.950585413 | 0.021499399 |
| 1074 | 190 | K190M | 0.950631236 | 0.018643496 |
| 1075 | 126 | P1265 | 0.950633844 | 0.021499658 |
| 1076 | 276 | Q276H | 0.95065036 | 0.026297352 |
| 1077 | 287 | G287E | 0.950768845 | 0.018644138 |
| 1078 | 3 | A3S | 0.950841255 | 0.021500765 |
| 1079 | 215 | D215E | 0.950984396 | 0.013252542 |
| 1080 | 127 | K127T | 0.951071576 | 0.018645551 |
| 1081 | 58 | N58Y | 0.951189253 | 0.021502622 |
| 1082 | 164 | F164C | 0.951332182 | 0.026301765 |
| 1083 | 296 | 2296 | 0.95163624 | 0.021505007 |
| 1084 | 118 | F118Y | 0.951751748 | 0.037151408 |
| 1085 | 142 | L142I | 0.951781604 | 0.026304673 |
| 1086 | 40 | L40V | 0.951784531 | 0.026304692 |
| 1087 | 218 | G218A | 0.951854804 | 0.037152341 |
| 1088 | 96 | G96V | 0.952078108 | 0.026306591 |
| 1089 | 267 | M267V | 0.952212962 | 0.021508083 |
| 1090 | 284 | N284D | 0.952221024 | 0.026307515 |
| 1091 | 88 | L88I | 0.952391066 | 0.021509032 |
| 1092 | 167 | D167H | 0.952403012 | 0.037157306 |
| 1093 | 8 | V8D | 0.953062745 | 0.037163277 |
| 1094 | 185 | P185L | 0.953084386 | 0.026313096 |
| 1095 | 244 | P244A | 0.953247593 | 0.021513598 |
| 1096 | 277 | T277S | 0.953376343 | 0.018656298 |
| 1097 | 310 | A310V | 0.953806607 | 0.026317763 |
| 1098 | 5 | R5K | 0.953843602 | 0.021516773 |
| 1099 | 330 | L330M | 0.953850794 | 0.026318048 |
| 1100 | 164 | F164L | 0.953852179 | 0.021516819 |
| 1101 | 217 | L217P | 0.953886831 | 0.021517003 |
| 1102 | 343 | A343P | 0.953899217 | 0.018658734 |
| 1103 | 141 | N141S | 0.95402289 | 0.02631916 |
| 1104 | 58 | N58H | 0.954061023 | 0.018659487 |
| 1105 | 31 | D31V | 0.954454341 | 0.021520026 |
| 1106 | 312 | G312E | 0.954551115 | 0.021520541 |
| 1107 | 270 | L270F | 0.954731155 | 0.018662608 |
| 1108 | 237 | S237C | 0.954909324 | 0.014163536 |
| 1109 | 268 | E268G | 0.955012452 | 0.01671536 |
| 1110 | 324 | A324S | 0.955141013 | 0.026326379 |
| 1111 | 74 | F74V | 0.955215407 | 0.026326859 |
| 1112 | 314 | D314Y | 0.955317963 | 0.018665339 |
| 1113 | 2 | D2V | 0.955355613 | 0.021524823 |
| 1114 | 370 | L370I | 0.955451543 | 0.037184877 |
| 1115 | 55 | N55S | 0.9554669 | 0.026328482 |
| 1116 | 25 | I25L | 0.955564402 | 0.021525934 |
| 1117 | 366 | E366D | 0.955614711 | 0.026329436 |
| 1118 | 345 | L345P | 0.955939426 | 0.02633153 |
| 1119 | 265 | A265P | 0.955974058 | 0.026331754 |
| 1120 | 17 | L17V | 0.956074698 | 0.026332403 |
| 1121 | 315 | A315P | 0.9561852 | 0.026333115 |
| 1122 | 333 | K333R | 0.956483683 | 0.02633504 |
| 1123 | 363 | K363E | 0.95652276 | 0.018670943 |
| 1124 | 254 | V254L | 0.956595389 | 0.01528501 |
| 1125 | 369 | Q369P | 0.95683011 | 0.026337274 |
| 1126 | 86 | V86A | 0.956865093 | 0.018672535 |
| 1127 | 120 | T120I | 0.95707084 | 0.026338825 |
| 1128 | 254 | V254A | 0.95742097 | 0.037202657 |
| 1129 | 48 | D48V | 0.957426516 | 0.037202707 |
| 1130 | 70 | Y70F | 0.957500825 | 0.037203377 |
| 1131 | 268 | E268Q | 0.958052865 | 0.026345153 |
| 1132 | 261 | N261T | 0.958116164 | 0.02634556 |
| 1133 | 309 | N309T | 0.958142166 | 0.016728501 |
| 1134 | 244 | P244Q | 0.958269745 | 0.021540314 |
| 1135 | 269 | K269M | 0.95848191 | 0.026347916 |
| 1136 | 92 | G92D | 0.958742688 | 0.015293314 |
| 1137 | 152 | C152S | 0.958867181 | 0.013279487 |
| 1138 | 141 | N141Y | 0.958893878 | 0.037215938 |
| 1139 | 238 | S238Y | 0.958908311 | 0.018682026 |
| 1140 | 218 | G218R | 0.958930139 | 0.02154382 |
| 1141 | 9 | N9S | 0.958974716 | 0.037216667 |
| 1142 | 232 | V232L | 0.958980954 | 0.021544089 |
| 1143 | 258 | I258L | 0.959019105 | 0.018682541 |
| 1144 | 284 | N284S | 0.959416685 | 0.026353933 |
| 1145 | 256 | E256D | 0.959666914 | 0.026355543 |
| 1146 | 84 | S84N | 0.959882106 | 0.026356927 |
| 1147 | 369 | Q369H | 0.959909501 | 0.026357103 |
| 1148 | 146 | A146S | 0.960068632 | 0.026358127 |
| 1149 | 231 | K231E | 0.960349249 | 0.018688712 |
| 1150 | 254 | V254G | 0.960395367 | 0.016737946 |
| 1151 | 368 | H368D | 0.960478449 | 0.015300018 |
| 1152 | 253 | L253W | 0.9604852 | 0.026360806 |
| 1153 | 256 | E256V | 0.960534533 | 0.016738529 |
| 1154 | 262 | G262E | 0.960549329 | 0.026361218 |
| 1155 | 306 | R306S | 0.960573863 | 0.03723107 |
| 1156 | 233 | Q233K | 0.960844305 | 0.021553974 |
| 1157 | 278 | I278M | 0.960963898 | 0.021554608 |
| 1158 | 294 | E294V | 0.961004027 | 0.016740495 |
| 1159 | 174 | V174A | 0.961198156 | 0.026365389 |
| 1160 | 261 | N261I | 0.961298927 | 0.037237595 |
| 1161 | 43 | S43C | 0.961321875 | 0.018693222 |
| 1162 | 71 | K71R | 0.96133491 | 0.026366268 |
| 1163 | 138 | S138C | 0.961436462 | 0.018693753 |
| 1164 | 325 | L325P | 0.961443101 | 0.021557148 |
| 1165 | 322 | D322G | 0.961582909 | 0.01530428 |
| 1166 | 2 | D2G | 0.961605933 | 0.02155801 |
| 1167 | 358 | A358T | 0.961850181 | 0.01869567 |
| 1168 | 106 | A106V | 0.962066233 | 0.021560449 |
| 1169 | 269 | K269N | 0.962101804 | 0.018696836 |
| 1170 | 232 | V232E | 0.962108481 | 0.018696867 |
| 1171 | 114 | E114G | 0.962220484 | 0.026371957 |
| 1172 | 360 | F360I | 0.962258105 | 0.037246222 |
| 1173 | 295 | P295A | 0.962273806 | 0.037246363 |
| 1174 | 253 | L253V | 0.962450021 | 0.021562482 |
| 1175 | 210 | V210G | 0.962586578 | 0.026374308 |
| 1176 | 124 | V124I | 0.962752502 | 0.01869985 |
| 1177 | 123 | I123M | 0.963077613 | 0.02637746 |
| 1178 | 207 | V207G | 0.963304694 | 0.015310919 |
| 1179 | 278 | I278V | 0.963538479 | 0.016751098 |
| 1180 | 318 | K318I | 0.963632924 | 0.021568743 |
| 1181 | 246 | F246S | 0.963977579 | 0.026383235 |
| 1182 | 113 | E113D | 0.964097785 | 0.026384006 |
| 1183 | 3 | A3T | 0.964230445 | 0.01870669 |
| 1184 | 27 | K27M | 0.964585081 | 0.015315851 |
| 1185 | 46 | S46L | 0.964678512 | 0.037267966 |
| 1186 | 327 | L327R | 0.964730101 | 0.016756077 |
| 1187 | 218 | G218E | 0.964736049 | 0.018709029 |
| 1188 | 88 | L88S | 0.965067504 | 0.021576331 |
| 1189 | 65 | A65G | 0.965296788 | 0.021577542 |
| 1190 | 286 | Q286E | 0.965371228 | 0.02639217 |
| 1191 | 296 | Q296K | 0.965393531 | 0.026392313 |
| 1192 | 184 | K184R | 0.965398106 | 0.026392343 |
| 1193 | 18 | P18S | 0.965573413 | 0.0187129 |
| 1194 | 6 | Q6R | 0.965607577 | 0.016759741 |
| 1195 | 360 | F360Y | 0.965617431 | 0.037276391 |
| 1196 | 48 | D48A | 0.965628565 | 0.037276491 |
| 1197 | 146 | A146V | 0.965710173 | 0.026394342 |
| 1198 | 273 | I273L | 0.965736257 | 0.018713653 |
| 1199 | 332 | L332V | 0.965863021 | 0.037278594 |
| 1200 | 141 | N141D | 0.96586447 | 0.021580542 |
| 1201 | 168 | V168A | 0.966007208 | 0.021581296 |
| 1202 | 232 | V232G | 0.966077393 | 0.021581667 |
| 1203 | 53 | I53V | 0.966166399 | 0.016762074 |
| 1204 | 59 | L59R | 0.966209321 | 0.02639754 |
| 1205 | 277 | T277K | 0.966312363 | 0.0263982 |
| 1206 | 328 | N328D | 0.966322884 | 0.016762727 |
| 1207 | 233 | Q233H | 0.966336837 | 0.016762785 |
| 1208 | 272 | S272C | 0.966344602 | 0.021583079 |
| 1209 | 52 | I52S | 0.966363646 | 0.037283083 |
| 1210 | 75 | L75Q | 0.966381894 | 0.021583276 |
| 1211 | 130 | S130N | 0.966892162 | 0.016765102 |
| 1212 | 327 | L327F | 0.967063306 | 0.037289354 |
| 1213 | 141 | N141K | 0.967116658 | 0.026403349 |
| 1214 | 322 | D322N | 0.96720097 | 0.021587601 |
| 1215 | 138 | S138I | 0.967327291 | 0.018721002 |
| 1216 | 314 | D314H | 0.967609245 | 0.014209335 |
| 1217 | 151 | Y151H | 0.967799585 | 0.015328215 |
| 1218 | 142 | L142R | 0.968112904 | 0.021592413 |
| 1219 | 274 | K274N | 0.968237545 | 0.026410521 |
| 1220 | 96 | G96A | 0.968453021 | 0.016771609 |
| 1221 | 40 | L40F | 0.968487809 | 0.015330859 |
| 1222 | 234 | A234S | 0.968570583 | 0.021594827 |
| 1223 | 184 | K184E | 0.968752037 | 0.037304477 |
| 1224 | 58 | N58S | 0.968853459 | 0.016773277 |
| 1225 | 182 | L182V | 0.968947549 | 0.026415061 |
| 1226 | 294 | E294K | 0.968976012 | 0.037306482 |
| 1227 | 301 | M301K | 0.969111308 | 0.021597678 |
| 1228 | 12 | P12H | 0.969177922 | 0.01872954 |
| 1229 | 327 | L327V | 0.969395579 | 0.037310236 |
| 1230 | 362 | K362T | 0.96983496 | 0.021601492 |
| 1231 | 91 | I91L | 0.969849762 | 0.011401715 |
| 1232 | 81 | G81A | 0.969923352 | 0.026421297 |
| 1233 | 73 | I73T | 0.969997313 | 0.021602348 |
| 1234 | 351 | I351S | 0.970045827 | 0.02642208 |
| 1235 | 362 | K362Q | 0.97012301 | 0.02160301 |
| 1236 | 40 | L40H | 0.970328068 | 0.015337923 |
| 1237 | 330 | L330V | 0.970349507 | 0.026424019 |
| 1238 | 26 | Q26L | 0.970375164 | 0.026424183 |
| 1239 | 309 | N309Y | 0.970416492 | 0.018735249 |
| 1240 | 212 | V212A | 0.97059115 | 0.015338932 |
| 1241 | 266 | A266D | 0.970608382 | 0.026425672 |
| 1242 | 308 | G308S | 0.970934915 | 0.026427757 |
| 1243 | 360 | F360C | 0.970975866 | 0.021607502 |
| 1244 | 111 | A111G | 0.970984987 | 0.026428077 |
| 1245 | 68 | D68H | 0.971110932 | 0.021608213 |
| 1246 | 247 | S247N | 0.9711902 | 0.018738813 |
| 1247 | 50 | A50D | 0.971252901 | 0.037326841 |
| 1248 | 331 | S331P | 0.971301893 | 0.0264301 |
| 1249 | 363 | K363Q | 0.971461518 | 0.021610059 |
| 1250 | 326 | E326A | 0.971545933 | 0.037329459 |
| 1251 | 362 | K362N | 0.971653057 | 0.021611067 |
| 1252 | 33 | K33N | 0.971679124 | 0.026432507 |
| 1253 | 229 | E229V | 0.971693711 | 0.018741131 |
| 1254 | 249 | Y249D | 0.971718148 | 0.026432756 |
| 1255 | 261 | N261K | 0.971753821 | 0.021611597 |
| 1256 | 27 | K27N | 0.971999645 | 0.026434552 |
| 1257 | 170 | G170R | 0.97200195 | 0.01874255 |
| 1258 | 311 | K311N | 0.972019864 | 0.018742633 |
| 1259 | 220 | A220S | 0.972175694 | 0.021613817 |
| 1260 | 4 | P4A | 0.972313959 | 0.018743986 |
| 1261 | 278 | I278L | 0.972344813 | 0.021614707 |
| 1262 | 112 | A112V | 0.972438958 | 0.026437354 |
| 1263 | 273 | I273N | 0.972494508 | 0.026437709 |
| 1264 | 132 | T132P | 0.9725805 | 0.037338698 |
| 1265 | 221 | L221F | 0.972600757 | 0.018745306 |
| 1266 | 237 | S237T | 0.972757277 | 0.018746026 |
| 1267 | 214 | D214H | 0.973518447 | 0.018749527 |
| 1268 | 142 | L142P | 0.973553855 | 0.037347383 |
| 1269 | 218 | G218V | 0.97360174 | 0.021621316 |
| 1270 | 36 | G36D | 0.973627162 | 0.026444929 |
| 1271 | 341 | I341F | 0.973630112 | 0.021621465 |
| 1272 | 268 | E268D | 0.973715537 | 0.026445492 |
| 1273 | 142 | L142F | 0.97382194 | 0.016793942 |
| 1274 | 308 | G308R | 0.973831603 | 0.018750966 |
| 1275 | 315 | A315S | 0.973903844 | 0.021622904 |
| 1276 | 31 | D31N | 0.974205209 | 0.026448612 |
| 1277 | 264 | A264P | 0.974207897 | 0.01535279 |
| 1278 | 306 | R306L | 0.974308562 | 0.018753159 |
| 1279 | 47 | S47A | 0.974403952 | 0.021625532 |
| 1280 | 34 | G34V | 0.974438845 | 0.0264501 |
| 1281 | 211 | I211V | 0.974452842 | 0.026450189 |
| 1282 | 258 | I258N | 0.974554342 | 0.021626322 |
| 1283 | 223 | E223Q | 0.974743837 | 0.018755159 |
| 1284 | 284 | N284H | 0.974751765 | 0.021627359 |
| 1285 | 50 | A50S | 0.974759614 | 0.0216274 |
| 1286 | 295 | P295S | 0.974804922 | 0.018755439 |
| 1287 | 6 | Q6E | 0.974860759 | 0.021627931 |
| 1288 | 358 | A358G | 0.974997578 | 0.016798823 |
| 1289 | 321 | L321H | 0.975017204 | 0.026453783 |
| 1290 | 214 | D214A | 0.975214151 | 0.021629787 |
| 1291 | 205 | A205S | 0.975263607 | 0.037362626 |
| 1292 | 188 | V188I | 0.975408366 | 0.037363916 |
| 1293 | 240 | Y240F | 0.975556769 | 0.01535795 |
| 1294 | 273 | I273M | 0.975954754 | 0.016802793 |
| 1295 | 118 | F118L | 0.976029238 | 0.037369446 |
| 1296 | 313 | D313E | 0.97629121 | 0.026461891 |
| 1297 | 314 | D314G | 0.976428043 | 0.016804756 |
| 1298 | 36 | G36A | 0.976466976 | 0.026463009 |
| 1299 | 273 | I273V | 0.976531989 | 0.021636703 |
| 1300 | 122 | N122T | 0.976605465 | 0.037374576 |
| 1301 | 319 | R319I | 0.976687564 | 0.037375307 |
| 1302 | 65 | A65S | 0.976710476 | 0.021637639 |
| 1303 | 291 | C291G | 0.976716569 | 0.015362383 |
| 1304 | 34 | G34R | 0.976771623 | 0.02163796 |
| 1305 | 219 | F219Y | 0.977014955 | 0.037378221 |
| 1306 | 271 | S271R | 0.977037993 | 0.02646664 |
| 1307 | 276 | Q276R | 0.977496562 | 0.026469555 |
| 1308 | 42 | M42L | 0.977536752 | 0.02646981 |
| 1309 | 339 | G339R | 0.977719773 | 0.014245541 |
| 1310 | 57 | E57Q | 0.977731391 | 0.021642993 |
| 1311 | 168 | V168G | 0.977888448 | 0.021643816 |
| 1312 | 18 | P18L | 0.977894788 | 0.037386048 |
| 1313 | 169 | K169N | 0.977910932 | 0.021643934 |
| 1314 | 322 | D32ZE | 0.978035729 | 0.037387301 |
| 1315 | 310 | A310P | 0.978123467 | 0.021645048 |
| 1316 | 113 | E113A | 0.978488422 | 0.026475857 |
| 1317 | 66 | V66I | 0.978716686 | 0.037393356 |
| 1318 | 259 | K259I | 0.978770474 | 0.037393834 |
| 1319 | 331 | S331A | 0.978829782 | 0.026478024 |
| 1320 | 260 | N260H | 0.978867754 | 0.021648947 |
| 1321 | 29 | L295 | 0.978935885 | 0.015370858 |
| 1322 | 123 | I123L | 0.978984787 | 0.02164956 |
| 1323 | 52 | I52F | 0.979007591 | 0.037395941 |
| 1324 | 337 | S337T | 0.979037676 | 0.018774859 |
| 1325 | 367 | M367R | 0.979054773 | 0.026479453 |
| 1326 | 90 | L90V | 0.979156576 | 0.018775404 |
| 1327 | 231 | K231Q | 0.979219254 | 0.026480497 |
| 1328 | 219 | F219V | 0.979409728 | 0.016817106 |
| 1329 | 127 | K127Q | 0.979530853 | 0.026482475 |
| 1330 | 214 | D214G | 0.979975611 | 0.018779155 |
| 1331 | 126 | P126H | 0.980115228 | 0.016820024 |
| 1332 | 324 | A324T | 0.980142901 | 0.016820139 |
| 1333 | 194 | I194T | 0.98024432 | 0.037406927 |
| 1334 | 249 | Y249S | 0.980314423 | 0.021656521 |
| 1335 | 76 | Q76P | 0.980397782 | 0.021656957 |
| 1336 | 37 | I37S | 0.980501611 | 0.01537683 |
| 1337 | 145 | D145N | 0.980545726 | 0.026488914 |
| 1338 | 77 | G77A | 0.980558228 | 0.026488994 |
| 1339 | 314 | D314E | 0.980759579 | 0.016822689 |
| 1340 | 294 | E294Q | 0.980771396 | 0.018782798 |
| 1341 | 127 | K127E | 0.980992864 | 0.016823653 |
| 1342 | 313 | D313G | 0.981103048 | 0.018784315 |
| 1343 | 138 | S138N | 0.98122489 | 0.026493221 |
| 1344 | 324 | A324G | 0.981283213 | 0.037416148 |
| 1345 | 48 | D48H | 0.981476478 | 0.037417863 |
| 1346 | 240 | Y240C | 0.981567088 | 0.021663073 |
| 1347 | 68 | D68N | 0.981590156 | 0.013356444 |
| 1348 | 129 | G129A | 0.981631029 | 0.026495796 |
| 1349 | 281 | I281L | 0.981743666 | 0.021663997 |
| 1350 | 202 | V202F | 0.98174705 | 0.037420263 |
| 1351 | 365 | L365S | 0.981898968 | 0.018787956 |
| 1352 | 322 | D322A | 0.981958116 | 0.026497869 |
| 1353 | 280 | E280D | 0.98217378 | 0.021666245 |
| 1354 | 273 | I273F | 0.98222007 | 0.037424458 |
| 1355 | 110 | K110R | 0.98240174 | 0.016829475 |
| 1356 | 27 | K27E | 0.982403881 | 0.021667447 |
| 1357 | 161 | E161A | 0.982483562 | 0.026501198 |
| 1358 | 201 | N201S | 0.982532222 | 0.026501506 |
| 1359 | 219 | F219L | 0.98263068 | 0.037428098 |
| 1360 | 193 | V193A | 0.98288426 | 0.016831467 |
| 1361 | 239 | L239W | 0.983193617 | 0.026505694 |
| 1362 | 363 | K363T | 0.983208619 | 0.018793943 |
| 1363 | 269 | K269T | 0.983249251 | 0.018794129 |
| 1364 | 248 | I248V | 0.983566643 | 0.018795579 |
| 1365 | 143 | N143D | 0.983723673 | 0.02650905 |
| 1366 | 31 | D31G | 0.983971315 | 0.018797427 |
| 1367 | 355 | Q355L | 0.983996355 | 0.021675765 |
| 1368 | 280 | E280Q | 0.984054349 | 0.026511142 |
| 1369 | 196 | A196V | 0.984571077 | 0.018800166 |
| 1370 | 93 | L93W | 0.984682886 | 0.026515119 |
| 1371 | 257 | W257L | 0.984739984 | 0.021679646 |
| 1372 | 323 | K323T | 0.984908403 | 0.026516545 |
| 1373 | 82 | Q82H | 0.985048176 | 0.026517429 |
| 1374 | 250 | V250L | 0.985149099 | 0.018802804 |
| 1375 | 272 | S272Y | 0.985169419 | 0.021681886 |
| 1376 | 252 | G252D | 0.985318806 | 0.02651914 |
| 1377 | 313 | D313H | 0.985344068 | 0.021682797 |
| 1378 | 304 | P304R | 0.985427785 | 0.026519829 |
| 1379 | 270 | L270H | 0.985433163 | 0.021683262 |
| 1380 | 6 | Q6P | 0.985707924 | 0.016843116 |
| 1381 | 264 | A264S | 0.985742049 | 0.026521815 |
| 1382 | 211 | I211M | 0.985830839 | 0.026522377 |
| 1383 | 128 | L128I | 0.985940098 | 0.015397528 |
| 1384 | 309 | N309K | 0.985996572 | 0.026523424 |
| 1385 | 86 | V86I | 0.986045311 | 0.026523732 |
| 1386 | 122 | N122D | 0.986071984 | 0.016844616 |
| 1387 | 55 | N55Y | 0.986248105 | 0.026525013 |
| 1388 | 367 | M367V | 0.987121212 | 0.018811798 |
| 1389 | 19 | H19N | 0.987349954 | 0.021693252 |
| 1390 | 172 | V172E | 0.987364171 | 0.018812905 |
| 1391 | 368 | H368N | 0.987514919 | 0.026533014 |
| 1392 | 184 | K184T | 0.987534829 | 0.037471499 |
| 1393 | 16 | K16T | 0.987738912 | 0.021695278 |
| 1394 | 136 | D136N | 0.987754971 | 0.018814686 |
| 1395 | 97 | R97K | 0.987884712 | 0.021696037 |
| 1396 | 266 | A266T | 0.987892893 | 0.021696079 |
| 1397 | 336 | R336T | 0.988368076 | 0.026538399 |
| 1398 | 260 | N260I | 0.988502797 | 0.016854625 |
| 1399 | 260 | N260Y | 0.98854398 | 0.037480411 |
| 1400 | 2 | D2N | 0.988667908 | 0.018818844 |
| 1401 | 272 | S272F | 0.988773246 | 0.021700662 |
| 1402 | 351 | I351L | 0.988892704 | 0.021701284 |
| 1403 | 329 | M329L | 0.989137241 | 0.037485648 |
| 1404 | 321 | L321V | 0.98929897 | 0.037487075 |
| 1405 | 343 | A343V | 0.989790822 | 0.026547371 |
| 1406 | 129 | G129E | 0.989821963 | 0.018824097 |
| 1407 | 54 | N54K | 0.98989876 | 0.021706517 |

### Example 3. Method of Diagnosis and Treatment of PSAT1 Deficiency in a Subject

A subject is identified as potentially having a genetic disorder related to PSATD. The functional status of PSAT1 in the subject is determined. If the subject exhibits at least one mutation that results in a protein variant of reduced activity and/or stability in PSAT1 as listed in Table 3, the subject is diagnosed as having PSATD. A composition comprising an effective amount of one or more mutants that result in one or more protein variants of increased activity and/or stability listed in Table 2 is administered to the subject via gene therapy. The subject is continuously monitored for reduction of symptoms associated with PSATD and the treatment is continued until there is an increase in the function of PSAT1 and the occurrence and/or symptoms of the disorder are reduced, alleviated, or limited.

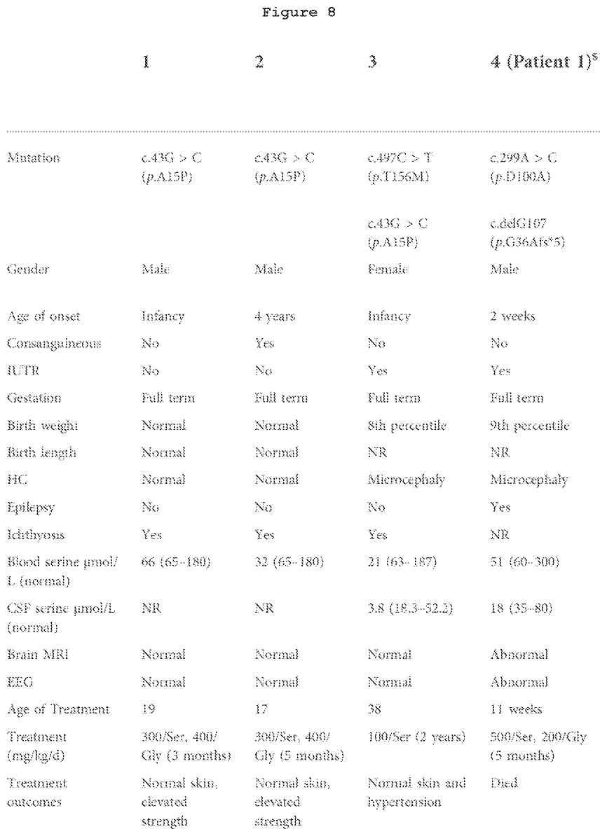

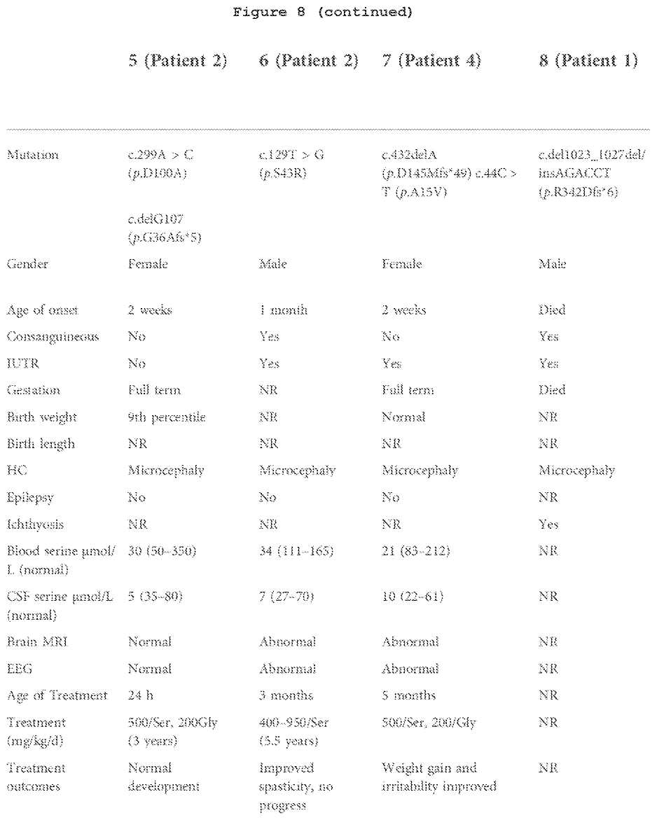

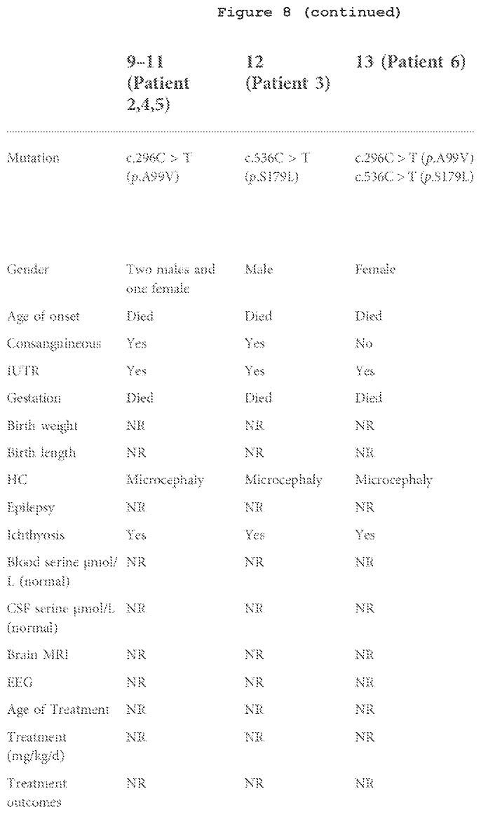

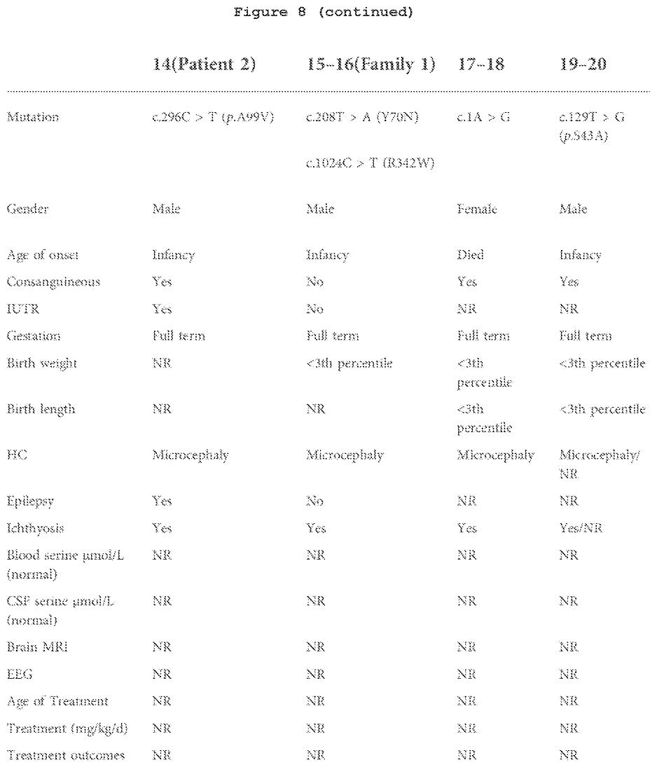

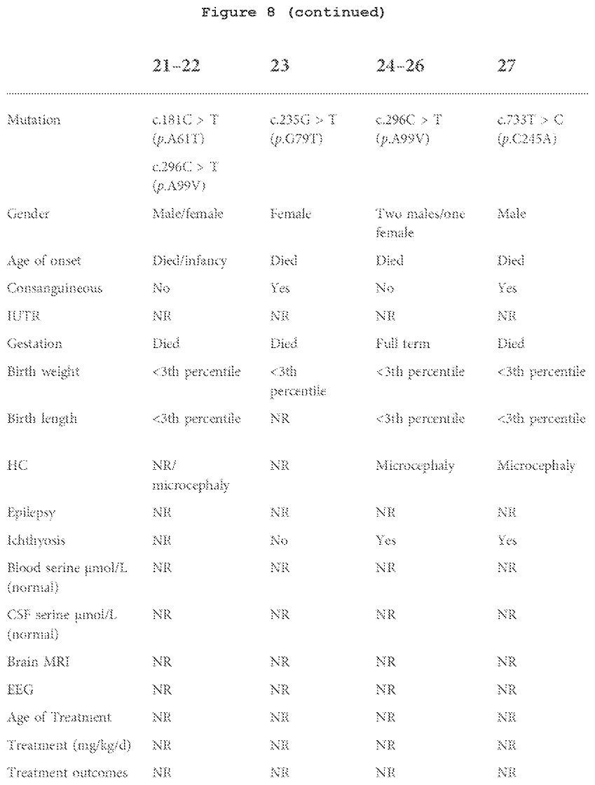

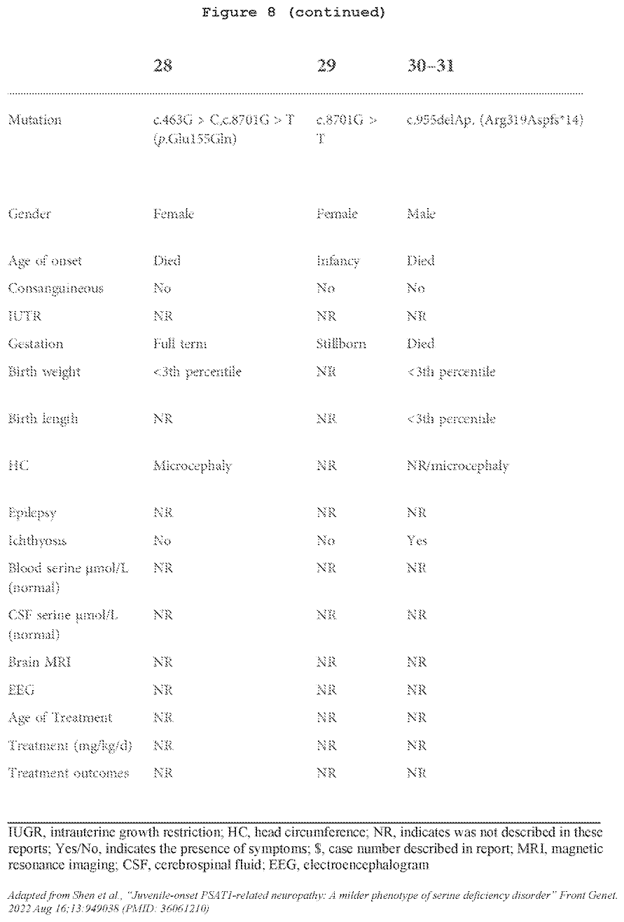

Alternatively, or in conjunction with the above therapies, dietary supplements may be administered to the subject. Several non-limiting examples of treatment outcomes are shown in FIG. 8, which is adapted from Shen et al., “Juvenile-onset PSAT1-related neuropathy: A milder phenotype of serine deficiency disorder” Front Genet. 2022 Aug. 16; 13:949038 (PMID: 36061210). As shown in FIG. 8, treatment outcomes 1-3 involved PSAT1 mutations that resulted in the condition known as ichthyosis, which is characterized by dry, itchy skin that appears scaly, rough, and red. As reported in FIG. 8, dietary supplementation that included the combination of serine and glycine, or serine alone, lead to normal skin phenotypes in treatment outcomes 1-3.

In a non-limiting example involving the variant D100A/G36A fs*5 of the PSAT1 gene (PMID: 17436247), dietary supplements were administered to siblings, one older and one younger. The older sibling was healthy at birth but started to have seizures at age of 7 weeks. At 11 weeks of age, 500 mg/kg/day serine and 200 mg/kg/day glycine were administered to the older sibling for 5 months. However, the older sibling eventually died at the age of 7 months. The younger sibling started to receive the serine/glycine treatment at an earlier age than the older sibling. 500 mg/kg/day serine, 200 mg/kg/day glycine was administered to the younger sibling within 24 hours of birth and continued through follow up at age 3. This treatment following detection of the variant in the younger sibling led to a developmentally normal outcome at the age of 3 years.

In another non-limiting example involving the detected variant S43R/S43R of the PSAT1 gene (PMID: 26610677), 500 mg/kg/day serine and 200 mg/kg/day glycine was administered to the subject at the age of 3 years and continued through the age of 5.5 years. Serine supplementation at 3 months of age improved spasticity of the subject. However, while the subject can establish eye contact and has various vocalizations, the subject's head circumference was small, there was no significant psychomotor progress, and the subject remained bedridden.

In another non-limiting example involving the detected variant A15V/D145M_fs*49 of the PSAT1 gene (PMID: 29269105), oral serine (500 mg/kg/d) and glycine (200 mg/kg/d) administration began at 5 months of age to the female subject. After the initiation of serine and glycine supplements, the subject saw her weight gain and irritability improved. However, the subject continued to have profound developmental delay. While some symptoms of serine deficiency, particularly those resulting from serine deficiency at an important developmental stage, may be irreversible via dietary supplementation, other symptoms (e.g., neuronal function or skin repair impairments) may still be correctable with dietary supplementation.

In another non-limiting example involving the detected variant A15P/A15P of the PSAT1 gene (PMID: 36061210), both juvenile subjects were given serine (300 mg/kg/d) and glycine (400 mg/kg/d) for 3-5 months. The subjects were unrelated and were diagnosed at juvenile age. Both subjects were born healthy. However, one had congenital ichthyosis and one manifested it at 4 years old (infancy-onset ichthyosis). Both subjects displayed neuropathy symptoms at 19 and 17 years old. The supplementation resolved ichthyosis and elevated strength in both subjects.

In another non-limiting example involving the variant A15P/T156M of the PSAT1 gene (PMID: 34089226) in an adult subject (38 years old), 100 mg/kg/day serine was administered and this supplement treatment continued through to the subject's follow up at age 40. The adult subject's serine supplement that began at age 38 resolved ichthyosis and reduced hypertension medication dosage. While it is possible that supplementation may have halted neuropathy progression, it did not appear to significantly reverse it.

As used herein, the section headings are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose, including the disclosures specifically referenced herein. When definitions of terms in incorporated references appear to differ from the definitions provided in the present teachings, the definition provided in the present teachings shall control. It will be appreciated that there is an implied “about” prior to the temperatures, concentrations, times, etc. discussed in the present teachings, such that slight and insubstantial deviations are within the scope of the present teachings herein.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.

In addition, where features or aspects of the disclosure are described in terms of Markush groups, those skilled in the art will recognize that the disclosure is also thereby described in terms of any individual member or subgroup of members of the Markush group.

Any of the features of an embodiment of the first through eleventh aspects is applicable to all aspects and embodiments identified herein. Moreover, any of the features of an embodiment of the first through eleventh aspects is independently combinable, partly or wholly with other embodiments described herein in any way, e.g., one, two, or three or more embodiments may be combinable in whole or in part. Further, any of the features of an embodiment of the first through eleventh aspects may be made optional to other aspects or embodiments.

1.-71. (canceled)

72. A method of identifying a subject having a genetic disorder, the method comprising:
detecting a presence of at least one mutation in PSAT1 protein that is different in the subject with a genetic disorder as compared to a subject without the disorder,
wherein the mutation is selected from a group consisting of: C245R, T242M, G206R, N89Y, G119E, G13R, L40R, I25K, G78V, L63S, G78D, Y148N, G79R, S147Y, I25R, P12L, G203R, I121K, G78A, V39D, H44Y, C152F, T156M, S179L, E155Q, S84R, G11R, D176H, G79W, A99D, G78R, C152R, G119R, D176A, G105V, K200N, Y151D, D176Y, S108P, G197D, Q82R, R45S, G78C, A111P, V86D, N154Y, G13S, Q199P, Y148H, R342Q, G159R, K200Q, Y148D, K200T, E155V, A115D, L182R, A111D, Y101D, G79E, K200R, G197R, C152Y, D176N, E155K, A198D, K200M, R97G, V188D, P14T, P12R, N89K, S38R, Y101N, P87R, G13C, L255P, T104P, N154D, A109P, L90P, Q82P, N302Y, P14L, P14H, Q199K, T156P, T156R, C224W, R61P, S178P, C152W, A112P, T242P, L357R, L90R, H335D, N302K, W107R, L345R, Y289D, S108L, Y148S, Y101S, K200E, L255R, V174D, G78S, M251R, N201Y, S147F, W107C, G203D, T242R, V102E, S299R, Q199L, G203V, T242K, S344Y, R213P, D176E, S299I, R342W, Y289S, V174F, W107L, S147P, L332W, S344F, M361R, Y70D, R342P, R306P, I278N, Y150N, P243R, C291F, R342L, V212D, G105A, T156A, H158P, W107S, Y148C, M361K, R45M, V157E, C175W, Y289N, R342G, V193E, Y346N, G340R, A99P, H44L, E155G, V202D, S38I, I194N, Y70N, S178F, V102G, S178Y, R45G, V72G, N89I, Y101C, Y289H, R45K, N241I, R45T, Y70S, Y346S, I91S, V103G, F10S, G334W, H335L, R213G, I278S, C175R, V149E, Y151N, N89D, I282N, N302H, C291R, Q199E, S179P, G340D, I307N, A115T, I91T, P243H, V72E, S183P, V149G, L332S, S285Y, Q76R, D353V, F305S, A198P, A99T, A324P, H44N, P244R, S272P, M329R, E155A, H44P, G11V, Y346C, L64P, V174G, G119V, C175F, G105E, N154I, T156S, S275P, L270P, A111V, G340C, F181S, S247R, H335P, E155D, H44Q, F191S, I303N, T104K, G119W, Q276P, P14A, L59P, N122I, L17R, A115V, P14S, G11W, S299C, L22W, F364L, R45W, N241Y, Y70C, L75R, N201H, G334V, I121R, P87H, Y230D, V124D, L216R, D187Y, P87L, S179A, G170V, Y289C, A358P, V293M, S178T, G287V, P67L, A205D, W140L, N154T, P292R, V293E, G339V, S275Y, M361T, Y150C, V60E, A343D, W140R, S204Y, F364S, V193G, F83Y, V86G, H335R, A99V, P292Q, Y151S, L321P, R213L, I211S, N89T, E161K, F320C, N180K, S147T, F10I, G263V, P292A, C245Y, I282S, P166L, F364C, F288C, G340S, P166T, I282T, P292S, I303F, E161V, I278T, A111T, G197V, A153P, P166H, C245F, R213C, L64R, M361V, H335Q, T104R, H335N, S344P, S285C, P166S, L216P, F320I, W140G, V338E, G206V, V66F, L228P, F364V, F195S, S43R, Q233P, W140C, Y279H, V188G, R61W, R298I, A146D, D283H, L63W, I303S, M42R, C224Y, S285T, V209E, F320L, F364I, V174L, L357Q, R298S, I307S, V103E, C175Y, E317D, R298T, A106T, I341N, L216Q, N180Y, E317G, T139P, V172G, D163V, A112E, S285A, A324D, E366V, I282F, H158Y, F181C, Y279D, A359P, F191V, N347Y, I281F, S275T, K274I, P12T, Y279F, I303M, G92V, P292L, P166A, R213S, V293A, M177K, V186G, R298K, D163Y, N328I, I134L, V210E, V60G, R298G, V202G, Y279S, D353A, G206E, V21E, I134T, N89S, A264D, C80R, T277P, D100Y, F49S, Q233R, F320Y, F162V, G197S, G262V, Y230S, I134N, A15D, K94I, H368R, Y70H, I134S, I307T, D353Y, A358D, E114K, V290G, V160M, V160G, F118S, Q286P, S204P, V124G, T56R, H158R, M329I, N347D, E161D, F288S, F162I, I248S, K333N, N180H, L182Q, R61L, F10V, T104I, A15P, F288V, K33I, W257S, K274E, M267K, N302D, I281N, F191L, G79A, N347T, D100G, I211T, F305L, F83C, E366K, I303T, L182P, C291S, V186E, V293L, M329V, L316S, I281S, F162S, D163H, D100V, F288I, M267R, A348G, D283V, A99G, S204F, P67R, R5G, P225L, Y230H, L17M, V124F, F162C, V102L, I134F, I278F, G77E, V21G, V160A, V209G, F288L, F360S, C224R, F181L, Y230C, N89H, S189F, I282M, F191C, D314A, L75V, F10C, S178C, S20L, T56P, P67A, R306G, N69I, I258T, I134M, K190E, E57K, S179T, P126A, F305Y, H158L, H335Y, I341L, F305V, E317K, T277R, F83I, P87S, P244L, F83V, F49C, D187G, D283G, L316V, P4L, D100A, K190N, A15V, K356R, A220D, E280K, S108T, Y32D, G263R, V354G, S84T, M361L, I307F, V250F, P225H, F74S, E268V, E114A, E366A, S299T, N284I, A234P, Y101H, D353G, V168D, G218W, P67T, A106P, C152G, P18Q, I194S, C98F, D68Y, A99S, L90F, V202A, N54I, V103M, V290L, P67Q, Y151C, F49L, N58K, Y230N, L327P, V338G, G203C, V102A, T242A, R61Q, F49V, V354A, P135L, K190T, I194F, A109V, H125D, D163A, S46P, Y101F, S337P, K51Q, L59V, N297T, V124A, I307M, A266P, E280V, G159A, C224F, V149A, K116R, N154H, G263D, I52M, Y240D, Y346H, E352V, E229D, F181V, V86F, G197A, D187V, A106G, N54T, F162L, F181I, K333Q, D136A, P67S, D136G, G339E, D68A, W257C, G262A, K274R, S84I, I248N, G159C, K71N, M42V, V349D, I282L, N328Y, L228R, Q76E, V212G, S84C, R222G, E326Q, V35F, S43I, V7G, S183T, Q76H, K190R, V66L, S130R, Q276E, F305I, L40I, G287R, T104S, A343G, D167V, G235V, A220V, V254D, G77V, V157G, R213H, L239V, S238P, D187H, N302S, S275C, G339A, L217M, A196T, A220P, T208I, D100E, S247C, A264G, E62V, E161Q, V290D, S183C, Q6H, D167Y, H19L, I165L, S299G, S20P, Y32F, C98W, C175S, A153T, I258S, E161G, P137R, N284Y, L216M, D187A, A343S, Y279N, K51R, I123F, V21A, S130T, G36C, G308D, V188F, P126R, D215V, K33R, V207F, D100H, S183Y, N328S, I91V, K127R, L217R, I91F, N143K, N201K, V21L, N297D, G262R, G312V, P135T, N69D, N55I, V60A, N69Y, V250D, I165R, K300T, N201I, K323N, I53N, S20T, Q276L, K190Q, L128V, S204C, D187N, F83L, D215N, D353N, H19Q, G119A, E57D, G334E, A265T, Y32H, I73S, S108A, V35I, G81S, V186M, I165K, S331T, L23I, E317Q, V39L, T139S, P126L, H19D, P18A, V212F, G81C, H158Q, L173P, A348V, L316F, N180I, E57V, G235E, P304A, D145V, E24D, A112T, K323R, Q296P, S43G, I351V, N69K, G334R, R222P, T120S, E28A, E366G, V168I, V72A, D353H, I341S, L93S, L142H, T139N, D31E, P137T, F181Y, N309D, V103A, L75P, A85V, H125P, R222L, D163E, D215H, K333E, G129W, N309I, Q369L, G170A, R306C, S247I, V103L, P18R, N347H, E57G, I25M, P244S, W257G, L325H, I273S, I123V, K300E, I53S, Q82L, L327H, L270R, G36V, A266V, V21M, I282V, K300M, S271G, S189P, A50G, C224S, V124L, I52L, T277A, E62Q, K323Q, D313Y, A171S, N328K, I52T, I165M, K27Q, K16R, E24G, F288Y, L255Q, A220T, F195C, T132R, Q286R, G92A, V188A, Q369R, H125L, E256Q, T56A, E268K, P137Q, N54D, L59I, E57A, S147A, K94E, K51M, S237N, V60L, L253M, N236H, G235A, I211F, K16Q, A65V, D283A, D163G, G287A, P225A, K127N, K33E, F364Y, L59H, V39F, A109G, A171V, V227D, T277I, L217Q, M177T, H19P, F164S, G263C, E223V, L216V, D48N, L217V, E229Q, P295L, E113V, F10L, F164I, I303V, S38G, K323E, N58T, N297H, M267T, R5T, V186A, N58I, K27T, A146P, G308A, K133I, P244T, G252V, C224G, G92S, G81V, V202L, V168L, V290I, T104A, P185A, Y240H, E28Q, L345M, G36R, A198V, S272T, K333I, I341M, G263S, E280G, K274T, I165T, D2A, Q296H, A109D, E114V, Q355H, K318E, V254I, D322V, A153S, C98Y, D215Y, I73N, N241T, N141H, K363N, V35L, T120P, I281V, P225S, V212I, A109S, D68G, K274Q, L221P, D215G, L30F, E24V, L64V, D100N, M177V, L128F, G92C, V160L, A198G, E28D, P87T, S147C, E268A, R5M, V290A, A85T, P225T, S247G, R319G, H19R, L365W, I307V, L142V, E223K, L128R, S183A, T132I, W257R, G312R, P135A, D313A, Q276K, P87A, S204A, S38C, K362R, V207L, V250I, S331Y, T139A, A3D, A65D, E294G, H368P, A146G, S237I, T139I, F164Y, N54Y, K184N, R61G, V102M, V60M, I307L, I52N, K363R, D187E, E317A, V149L, E62A, S130C, A265S, D145E, V202I, N122H, Q82E, L64Q, M267L, L59F, S130G, I248F, D145A, K16E, S237G, D167E, Y150H, Q286L, N297Y, A95E, K300N, S247T, K33Q, I123T, K269E, M251T, A153G, Y230F, P135R, F164V, A171P, D145Y, N328T, L270V, I25V, I121L, S189C, A266S, V35G, C98S, M301T, V212L, V7M, D167N, I121M, T350I, P295T, A358V, K190M, P126S, Q276H, G287E, A3S, D215E, K127T, N58Y, F164C, Q296E, F118Y, L142I, L40V, G218A, G96V, M267V, N284D, L88I, D167H, V8D, P185L, P244A, T277S, A310V, R5K, L330M, F164L, L217P, A343P, N141S, N58H, D31V, G312E, L270F, S237C, E268G, A324S, F74V, D314Y, D2V, L370I, N55S, I25L, E366D, L345P, A265P, L17V, A315P, K333R, K363E, V254L, Q369P, V86A, T120I, V254A, D48V, Y70F, E268Q, N261T, N309T, P244Q, K269M, G92D, C152S, N141Y, S238Y, G218R, N9S, V232L, I258L, N284S, E256D, S84N, Q369H, A146S, K231E, V254G, H368D, L253W, E256V, G262E, R306S, Q233K, I278M, E294V, V174A, N261I, S43C, K71R, S138C, L325P, D322G, D2G, A358T, A106V, K269N, V232E, E114G, F360I, P295A, L253V, V210G, V124I, I123M, V207G, I278V, K318I, F246S, E113D, A3T, K27M, S46L, L327R, G218E, L88S, A65G, Q286E, Q296K, K184R, P18S, Q6R, F360Y, D48A, A146V, I273L, L332V, N141D, V168A, V232G, I53V, L59R, T277K, N328D, Q233H, S272C, I52S, L75Q, S130N, L327F, N141K, D322N, S138I, D314H, Y151H, L142R, K274N, G96A, L40F, A234S, K184E, N58S, L182V, E294K, M301K, P12H, L327V, K362T, I91L, G81A, I73T, I351S, K362Q, L40H, L330V, Q26L, N309Y, V212A, A266D, G308S, F360C, A111G, D68H, S247N, A50D, S331P, K363Q, E326A, K362N, K33N, E229V, Y249D, N261K, K27N, G170R, K311N, A220S, P4A, I278L, A112V, I273N, T132P, L221F, S237T, D214H, L142P, G218V, G36D, I341F, E268D, L142F, G308R, A315S, D31N, A264P, R306L, S47A, G34V, I211V, I258N, E223Q, N284H, A50S, P295S, Q6E, A358G, L321H, D214A, A205S, V188I, Y240F, I273M, F118L, D313E, D314G, G36A, I273V, N122T, R319I, A65S, C291G, G34R, F219Y, S271R, Q276R, M42L, G339R, E57Q, V168G, P18L, K169N, D322E, A310P, E113A, V66I, K259I, S331A, N260H, L29S, I123L, I52F, S337T, M367R, L90V, K231Q, F219V, K127Q, D214G, P126H, A324T, I194T, Y249S, Q76P, I37S, D145N, G77A, D314E, E294Q, K127E, D313G, S138N, A324G, D48H, Y240C, D68N, G129A, I281L, V202F, L365S, D322A, E280D, I273F, K110R, K27E, E161A, N201S, F219L, V193A, L239W, K363T, K269T, I248V, N143D, D31G, Q355L, E280Q, A196V, L93W, W257L, K323T, Q82H, V250L, S272Y, G252D, D313H, P304R, L270H, Q6P, A264S, I211M, L128I, N309K, V86I, N122D, N55Y, M367V, H19N, V172E, H368N, K184T, K16T, D136N, R97K, A266T, R336T, N260I, N260Y, D2N, S272F, I351L, M329L, L321V, A343V, G129E, N54K, and wherein the presence of at least one mutation indicates the presence of a genetic disorder in the subject.

73. The method of claim 1, wherein the presence of at least one mutation in PSAT1 protein results in protein variants of reduced activity, stability, abundance, functionality, activity, specificity or a combination thereof.

74. The method of claim 1, wherein the genetic disorder is (PSAT1) deficiency (PSATD), Neu-Laxova Syndrome 2 (NLS2), ichthyosis, epilepsy, hypertension, retinal degeneration, or macular telangiectasia type 2.

75. The method of claim 1, wherein the subject is a fetus, neonate, juvenile or adult.

76. The method of claim 1, wherein the subject is genetically pre-disposed to developing the genetic disorder, is at the early stages of developing the genetic disorder, or is at an advanced stage of genetic disorder.

77. An expression vector comprising a polynucleotide sequence encoding for one or more of the proteins, wherein the protein has a sequence that is at least 80% identical to SEQ ID NO: 1 (WT Sequence), comprising one or more of the amino acid substitution mutations as follows: K110T, P12A, N284T, I73V, V250G, N236S, A3G, K133R, V354F, T132S, I341V, G203A, V72L, F219I, V354L, V250A, I37M, A359V, K94R, L93F, I73F, V193L, V349G, S138G, F74C, S344C, K94Q, K318Q, K94T, P243A, L239M, K259T, M42I, L221I, K356Q, V8F, V7E, I351M, L255M, V72M, D136E, R336S, L22S, N236D, K169Q, A146T, A15T, L325V, I37T, Y240N, I73M, L29V, K231N, L63M, K300R, Q369K, D2Y, Y249F, K110E, H125R, E24A, S226A, C245G, 153F, T350P, P144T, G334A, R97M, R5S, N236K, P4T, T242S, Q26R, R97T, N302I, Y148F, T56I, E24K, K169E, D68E, Y249C, L17Q, E229A, L63V, P4R, I37N, C80G, L357M, A310S, N55T, P144Q, A109T, A15S, A85G, D353E, D48G, N180D, K116Q, I121T, S46A, L253S, D31Y, V157L, K116T, F49Y, K311Q, V157M, P144R, L221R, D31A, A95P, K110M, I73L, K356T, A198S, C80W, N55H, A106D, I121V, K117N, 137L, K117M, K117R, V174I, K117Q, P4H, N55K, K116E, K71Q, 137F.

78. The expression vector of claim 1, wherein the protein comprising one or more of the amino acid substitution mutations exhibits higher activity when compared to the WT protein of SEQ ID NO: 1.

79. A method of improving, ameliorating, or treating a genetic disorder or a symptom thereof, the method comprising:
detecting the presence of at least one mutation in PSAT1 protein that is different in the subject with the genetic disorder as compared to a subject without the genetic disorder, wherein the presence of at least one mutation indicates the presence of the genetic disorder in the subject; and
administering to the subject a protein, wherein the protein has a sequence that is at least 80% identical to SEQ ID NO: 1 (WT Sequence), comprising one or more of the amino acid substitution mutations as follows: K110T, P12A, N284T, I73V, V250G, N236S, A3G, K133R, V354F, T132S, I341V, G203A, V72L, F219I, V354L, V250A, I37M, A359V, K94R, L93F, I73F, V193L, V349G, S138G, F74C, S344C, K94Q, K318Q, K94T, P243A, L239M, K259T, M42I, L221I, K356Q, V8F, V7E, I351M, L255M, V72M, D136E, R336S, L22S, N236D, K169Q, A146T, A15T, L325V, I37T, Y240N, I73M, L29V, K231N, L63M, K300R, Q369K, D2Y, Y249F, K110E, H125R, E24A, S226A, C245G, I53F, T350P, P144T, G334A, R97M, R5S, N236K, P4T, T242S, Q26R, R97T, N302I, Y148F, T56I, E24K, K169E, D68E, Y249C, L17Q, E229A, L63V, P4R, I37N, C80G, L357M, A310S, N55T, P144Q, A109T, A15S, A85G, D353E, D48G, N180D, K116Q, I121T, S46A, L253S, D31Y, V157L, K116T, F49Y, K311Q, V157M, P144R, L221R, D31A, A95P, K110M, I73L, K356T, A198S, C80W, N55H, A106D, I121V, K117N, I37L, K117M, K117R, V174I, K117Q, P4H, N55K, K116E, K71Q, 137F and
wherein the genetic disorder or a symptom thereof is reduced after administration.

80. The method of claim 1, wherein the at least one mutation in PSAT1 protein is selected from a group consisting of: C245R, T242M, G206R, N89Y, G119E, G13R, L40R, I25K, G78V, L63S, G78D, Y148N, G79R, S147Y, I25R, P12L, G203R, I121K, G78A, V39D, H44Y, C152F, T156M, S179L, E155Q, S84R, G11R, D176H, G79W, A99D, G78R, C152R, G119R, D176A, G105V, K200N, Y151D, D176Y, S108P, G197D, Q82R, R45S, G78C, A111P, V86D, N154Y, G13S, Q199P, Y148H, R342Q, G159R, K200Q, Y148D, K200T, E155V, A115D, L182R, A111D, Y101D, G79E, K200R, G197R, C152Y, D176N, E155K, A198D, K200M, R97G, V188D, P14T, P12R, N89K, S38R, Y101N, P87R, G13C, L255P, T104P, N154D, A109P, L90P, Q82P, N302Y, P14L, P14H, Q199K, T156P, T156R, C224W, R61P, S178P, C152W, A112P, T242P, L357R, L90R, H335D, N302K, W107R, L345R, Y289D, S108L, Y148S, Y101S, K200E, L255R, V174D, G78S, M251R, N201Y, S147F, W107C, G203D, T242R, V102E, S299R, Q199L, G203V, T242K, S344Y, R213P, D176E, S299I, R342W, Y289S, V174F, W107L, S147P, L332W, S344F, M361R, Y70D, R342P, R306P, I278N, Y150N, P243R, C291F, R342L, V212D, G105A, T156A, H158P, W107S, Y148C, M361K, R45M, V157E, C175W, Y289N, R342G, V193E, Y346N, G340R, A99P, H44L, E155G, V202D, S38I, I194N, Y70N, S178F, V102G, S178Y, R45G, V72G, N89I, Y101C, Y289H, R45K, N241I, R45T, Y70S, Y346S, I91S, V103G, F10S, G334W, H335L, R213G, I278S, C175R, V149E, Y151N, N89D, I282N, N302H, C291R, Q199E, S179P, G340D, I307N, A115T, I91T, P243H, V72E, S183P, V149G, L332S, S285Y, Q76R, D353V, F305S, A198P, A99T, A324P, H44N, P244R, S272P, M329R, E155A, H44P, G11V, Y346C, L64P, V174G, G119V, C175F, G105E, N154I, T156S, S275P, L270P, A111V, G340C, F181S, S247R, H335P, E155D, H44Q, F191S, I303N, T104K, G119W, Q276P, P14A, L59P, N122I, L17R, A115V, P14S, G11W, S299C, L22W, F364L, R45W, N241Y, Y70C, L75R, N201H, G334V, I121R, P87H, Y230D, V124D, L216R, D187Y, P87L, S179A, G170V, Y289C, A358P, V293M, S178T, G287V, P67L, A205D, W140L, N154T, P292R, V293E, G339V, S275Y, M361T, Y150C, V60E, A343D, W140R, S204Y, F364S, V193G, F83Y, V86G, H335R, A99V, P292Q, Y151S, L321P, R213L, I211S, N89T, E161K, F320C, N180K, S147T, F10I, G263V, P292A, C245Y, I282S, P166L, F364C, F288C, G340S, P166T, I282T, P292S, I303F, E161V, I278T, A111T, G197V, A153P, P166H, C245F, R213C, L64R, M361V, H335Q, T104R, H335N, S344P, S285C, P166S, L216P, F320I, W140G, V338E, G206V, V66F, L228P, F364V, F195S, S43R, Q233P, W140C, Y279H, V188G, R61W, R298I, A146D, D283H, L63W, I303S, M42R, C224Y, S285T, V209E, F320L, F364I, V174L, L357Q, R298S, I307S, V103E, C175Y, E317D, R298T, A106T, I341N, L216Q, N180Y, E317G, T139P, V172G, D163V, A112E, S285A, A324D, E366V, I282F, H158Y, F181C, Y279D, A359P, F191V, N347Y, I281F, S275T, K274I, P12T, Y279F, I303M, G92V, P292L, P166A, R213S, V293A, M177K, V186G, R298K, D163Y, N328I, I134L, V210E, V60G, R298G, V202G, Y279S, D353A, G206E, V21E, I134T, N89S, A264D, C80R, T277P, D100Y, F49S, Q233R, F320Y, F162V, G197S, G262V, Y230S, I134N, A15D, K94I, H368R, Y70H, I134S, I307T, D353Y, A358D, E114K, V290G, V160M, V160G, F118S, Q286P, S204P, V124G, T56R, H158R, M329I, N347D, E161D, F288S, F162I, I248S, K333N, N180H, L182Q, R61L, F10V, T104I, A15P, F288V, K33I, W257S, K274E, M267K, N302D, I281N, F191L, G79A, N347T, D100G, I211T, F305L, F83C, E366K, I303T, L182P, C291S, V186E, V293L, M329V, L316S, I281S, F162S, D163H, D100V, F288I, M267R, A348G, D283V, A99G, S204F, P67R, R5G, P225L, Y230H, L17M, V124F, F162C, V102L, I134F, I278F, G77E, V21G, V160A, V209G, F288L, F360S, C224R, F181L, Y230C, N89H, S189F, I282M, F191C, D314A, L75V, F10C, S178C, S20L, T56P, P67A, R306G, N69I, I258T, I134M, K190E, E57K, S179T, P126A, F305Y, H158L, H335Y, I341L, F305V, E317K, T277R, F83I, P87S, P244L, F83V, F49C, D187G, D283G, L316V, P4L, D100A, K190N, A15V, K356R, A220D, E280K, S108T, Y32D, G263R, V354G, S84T, M361L, I307F, V250F, P225H, F74S, E268V, E114A, E366A, S299T, N284I, A234P, Y101H, D353G, V168D, G218W, P67T, A106P, C152G, P18Q, I194S, C98F, D68Y, A99S, L90F, V202A, N54I, V103M, V290L, P67Q, Y151C, F49L, N58K, Y230N, L327P, V338G, G203C, V102A, T242A, R61Q, F49V, V354A, P135L, K190T, I194F, A109V, H125D, D163A, S46P, Y101F, S337P, K51Q, L59V, N297T, V124A, I307M, A266P, E280V, G159A, C224F, V149A, K116R, N154H, G263D, I52M, Y240D, Y346H, E352V, E229D, F181V, V86F, G197A, D187V, A106G, N54T, F162L, F181I, K333Q, D136A, P67S, D136G, G339E, D68A, W257C, G262A, K274R, S84I, I248N, G159C, K71N, M42V, V349D, I282L, N328Y, L228R, Q76E, V212G, S84C, R222G, E326Q, V35F, S43I, V7G, S183T, Q76H, K190R, V66L, S130R, Q276E, F305I, L40I, G287R, T104S, A343G, D167V, G235V, A220V, V254D, G77V, V157G, R213H, L239V, S238P, D187H, N302S, S275C, G339A, L217M, A196T, A220P, T208I, D100E, S247C, A264G, E62V, E161Q, V290D, S183C, Q6H, D167Y, H19L, I165L, S299G, S20P, Y32F, C98W, C175S, A153T, I258S, E161G, P137R, N284Y, L216M, D187A, A343S, Y279N, K51R, I123F, V21A, S130T, G36C, G308D, V188F, P126R, D215V, K33R, V207F, D100H, S183Y, N328S, I91V, K127R, L217R, I91F, N143K, N201K, V21L, N297D, G262R, G312V, P135T, N69D, N55I, V60A, N69Y, V250D, I165R, K300T, N201I, K323N, I53N, S20T, Q276L, K190Q, L128V, S204C, D187N, F83L, D215N, D353N, H19Q, G119A, E57D, G334E, A265T, Y32H, I73S, S108A, V35I, G81S, V186M, I165K, S331T, L23I, E317Q, V39L, T139S, P126L, H19D, P18A, V212F, G81C, H158Q, L173P, A348V, L316F, N180I, E57V, G235E, P304A, D145V, E24D, A112T, K323R, Q296P, S43G, I351V, N69K, G334R, R222P, T120S, E28A, E366G, V168I, V72A, D353H, I341S, L93S, L142H, T139N, D31E, P137T, F181Y, N309D, V103A, L75P, A85V, H125P, R222L, D163E, D215H, K333E, G129W, N309I, Q369L, G170A, R306C, S247I, V103L, P18R, N347H, E57G, I25M, P244S, W257G, L325H, I273S, I123V, K300E, I53S, Q82L, L327H, L270R, G36V, A266V, V21M, I282V, K300M, S271G, S189P, A50G, C224S, V124L, I52L, T277A, E62Q, K323Q, D313Y, A171S, N328K, I52T, I165M, K27Q, K16R, E24G, F288Y, L255Q, A220T, F195C, T132R, Q286R, G92A, V188A, Q369R, H125L, E256Q, T56A, E268K, P137Q, N54D, L59I, E57A, S147A, K94E, K51M, S237N, V60L, L253M, N236H, G235A, I211F, K16Q, A65V, D283A, D163G, G287A, P225A, K127N, K33E, F364Y, L59H, V39F, A109G, A171V, V227D, T277I, L217Q, M177T, H19P, F164S, G263C, E223V, L216V, D48N, L217V, E229Q, P295L, E113V, F10L, F164I, I303V, S38G, K323E, N58T, N297H, M267T, R5T, V186A, N58I, K27T, A146P, G308A, K133I, P244T, G252V, C224G, G92S, G81V, V202L, V168L, V290I, T104A, P185A, Y240H, E28Q, L345M, G36R, A198V, S272T, K333I, I341M, G263S, E280G, K274T, I165T, D2A, Q296H, A109D, E114V, Q355H, K318E, V254I, D322V, A153S, C98Y, D215Y, I73N, N241T, N141H, K363N, V35L, T120P, I281V, P225S, V212I, A109S, D68G, K274Q, L221P, D215G, L30F, E24V, L64V, D100N, M177V, L128F, G92C, V160L, A198G, E28D, P87T, S147C, E268A, R5M, V290A, A85T, P225T, S247G, R319G, H19R, L365W, I307V, L142V, E223K, L128R, S183A, T132I, W257R, G312R, P135A, D313A, Q276K, P87A, S204A, S38C, K362R, V207L, V250I, S331Y, T139A, A3D, A65D, E294G, H368P, A146G, S237I, T139I, F164Y, N54Y, K184N, R61G, V102M, V60M, I307L, I52N, K363R, D187E, E317A, V149L, E62A, S130C, A265S, D145E, V202I, N122H, Q82E, L64Q, M267L, L59F, S130G, I248F, D145A, K16E, S237G, D167E, Y150H, Q286L, N297Y, A95E, K300N, S247T, K33Q, I123T, K269E, M251T, A153G, Y230F, P135R, F164V, A171P, D145Y, N328T, L270V, I25V, I121L, S189C, A266S, V35G, C98S, M301T, V212L, V7M, D167N, I121M, T350I, P295T, A358V, K190M, P126S, Q276H, G287E, A3S, D215E, K127T, N58Y, F164C, Q296E, F118Y, L142I, L40V, G218A, G96V, M267V, N284D, L88I, D167H, V8D, P185L, P244A, T277S, A310V, R5K, L330M, F164L, L217P, A343P, N141S, N58H, D31V, G312E, L270F, S237C, E268G, A324S, F74V, D314Y, D2V, L370I, N55S, I25L, E366D, L345P, A265P, L17V, A315P, K333R, K363E, V254L, Q369P, V86A, T120I, V254A, D48V, Y70F, E268Q, N261T, N309T, P244Q, K269M, G92D, C152S, N141Y, S238Y, G218R, N9S, V232L, I258L, N284S, E256D, S84N, Q369H, A146S, K231E, V254G, H368D, L253W, E256V, G262E, R306S, Q233K, I278M, E294V, V174A, N261I, S43C, K71R, S138C, L325P, D322G, D2G, A358T, A106V, K269N, V232E, E114G, F360I, P295A, L253V, V210G, V124I, I123M, V207G, I278V, K318I, F246S, E113D, A3T, K27M, S46L, L327R, G218E, L88S, A65G, Q286E, Q296K, K184R, P18S, Q6R, F360Y, D48A, A146V, I273L, L332V, N141D, V168A, V232G, I53V, L59R, T277K, N328D, Q233H, S272C, I52S, L75Q, S130N, L327F, N141K, D322N, S138I, D314H, Y151H, L142R, K274N, G96A, L40F, A234S, K184E, N58S, L182V, E294K, M301K, P12H, L327V, K362T, I91L, G81A, I73T, I351S, K362Q, L40H, L330V, Q26L, N309Y, V212A, A266D, G308S, F360C, A111G, D68H, S247N, A50D, S331P, K363Q, E326A, K362N, K33N, E229V, Y249D, N261K, K27N, G170R, K311N, A220S, P4A, I278L, A112V, I273N, T132P, L221F, S237T, D214H, L142P, G218V, G36D, I341F, E268D, L142F, G308R, A315S, D31N, A264P, R306L, S47A, G34V, I211V, I258N, E223Q, N284H, A50S, P295S, Q6E, A358G, L321H, D214A, A205S, V188I, Y240F, I273M, F118L, D313E, D314G, G36A, I273V, N122T, R319I, A65S, C291G, G34R, F219Y, S271R, Q276R, M42L, G339R, E57Q, V168G, P18L, K169N, D322E, A310P, E113A, V66I, K259I, S331A, N260H, L29S, I123L, I52F, S337T, M367R, L90V, K231Q, F219V, K127Q, D214G, P126H, A324T, I194T, Y249S, Q76P, I37S, D145N, G77A, D314E, E294Q, K127E, D313G, S138N, A324G, D48H, Y240C, D68N, G129A, I281L, V202F, L365S, D322A, E280D, I273F, K110R, K27E, E161A, N201S, F219L, V193A, L239W, K363T, K269T, I248V, N143D, D31G, Q355L, E280Q, A196V, L93W, W257L, K323T, Q82H, V250L, S272Y, G252D, D313H, P304R, L270H, Q6P, A264S, I211M, L128I, N309K, V86I, N122D, N55Y, M367V, H19N, V172E, H368N, K184T, K16T, D136N, R97K, A266T, R336T, N260I, N260Y, D2N, S272F, I351L, M329L, L321V, A343V, G129E, N54K.

81. The method of claim 1, wherein the genetic disorder is (PSAT1) deficiency (PSATD), or Neu-Laxova Syndrome 2 (NLS2).

82. A method of enhancing the functionality of PSAT1 protein in a subject with (PSAT1) deficiency (PSATD) or a symptom thereof, or Neu-Laxova Syndrome 2 (NLS2) or a symptom thereof, the method comprising:
administering to the subject a protein has a sequence that is at least 80% identical to SEQ ID NO: 1 (WT Sequence), comprising one or more of the amino acid substitution mutations as follows: K110T, P12A, N284T, I73V, V250G, N236S, A3G, K133R, V354F, T132S, I341V, G203A, V72L, F219I, V354L, V250A, I37M, A359V, K94R, L93F, I73F, V193L, V349G, S138G, F74C, S344C, K94Q, K318Q, K94T, P243A, L239M, K259T, M42I, L221I, K356Q, V8F, V7E, I351M, L255M, V72M, D136E, R336S, L22S, N236D, K169Q, A146T, A15T, L325V, I37T, Y240N, I73M, L29V, K231N, L63M, K300R, Q369K, D2Y, Y249F, K110E, H125R, E24A, S226A, C245G, I53F, T350P, P144T, G334A, R97M, R5S, N236K, P4T, T242S, Q26R, R97T, N302I, Y148F, T56I, E24K, K169E, D68E, Y249C, L17Q, E229A, L63V, P4R, I37N, C80G, L357M, A310S, N55T, P144Q, A109T, A15S, A85G, D353E, D48G, N180D, K116Q, I121T, S46A, L253S, D31Y, V157L, K116T, F49Y, K311Q, V157M, P144R, L221R, D31A, A95P, K110M, I73L, K356T, A198S, C80W, N55H, A106D, I121V, K117N, I37L, K117M, K117R, V174I, K117Q, P4H, N55K, K116E, K71Q, 137F, wherein the functionality of PSAT1 protein is enhanced when compared to the function of WT protein of SEQ ID NO: 1.

83. The method of claim 1, wherein the PSATD or a symptom thereof, or NLS2 or a symptom thereof is reduced after administration.

84. A method of improving, ameliorating, or treating a genetic disorder or a symptom thereof, the method comprising:
detecting the presence of at least one mutation in PSAT1 protein that is different in the subject with the genetic disorder as compared to a subject without the genetic disorder, wherein the presence of at least one mutation indicates the presence of the genetic disorder in the subject; and
administering to the subject a treatment,
wherein the treatment comprises an effective amount of a dietary supplementation, and
wherein the genetic disorder or a symptom thereof is reduced after administration.

85. The method of claim 1, wherein the at least one mutation in PSAT1 protein is selected from a group consisting of: C245R, T242M, G206R, N89Y, G119E, G13R, L40R, I25K, G78V, L63S, G78D, Y148N, G79R, S147Y, I25R, P12L, G203R, I121K, G78A, V39D, H44Y, C152F, T156M, S179L, E155Q, S84R, G11R, D176H, G79W, A99D, G78R, C152R, G119R, D176A, G105V, K200N, Y151D, D176Y, S108P, G197D, Q82R, R45S, G78C, A111P, V86D, N154Y, G13S, Q199P, Y148H, R342Q, G159R, K200Q, Y148D, K200T, E155V, A115D, L182R, A111D, Y101D, G79E, K200R, G197R, C152Y, D176N, E155K, A198D, K200M, R97G, V188D, P14T, P12R, N89K, S38R, Y101N, P87R, G13C, L255P, T104P, N154D, A109P, L90P, Q82P, N302Y, P14L, P14H, Q199K, T156P, T156R, C224W, R61P, S178P, C152W, A112P, T242P, L357R, L90R, H335D, N302K, W107R, L345R, Y289D, S108L, Y148S, Y101S, K200E, L255R, V174D, G78S, M251R, N201Y, S147F, W107C, G203D, T242R, V102E, S299R, Q199L, G203V, T242K, S344Y, R213P, D176E, S299I, R342W, Y289S, V174F, W107L, S147P, L332W, S344F, M361R, Y70D, R342P, R306P, I278N, Y150N, P243R, C291F, R342L, V212D, G105A, T156A, H158P, W107S, Y148C, M361K, R45M, V157E, C175W, Y289N, R342G, V193E, Y346N, G340R, A99P, H44L, E155G, V202D, S38I, I194N, Y70N, S178F, V102G, S178Y, R45G, V72G, N89I, Y101C, Y289H, R45K, N241I, R45T, Y70S, Y346S, I91S, V103G, F10S, G334W, H335L, R213G, I278S, C175R, V149E, Y151N, N89D, I282N, N302H, C291R, Q199E, S179P, G340D, I307N, A115T, I91T, P243H, V72E, S183P, V149G, L332S, S285Y, Q76R, D353V, F305S, A198P, A99T, A324P, H44N, P244R, S272P, M329R, E155A, H44P, G11V, Y346C, L64P, V174G, G119V, C175F, G105E, N154I, T156S, S275P, L270P, A111V, G340C, F181S, S247R, H335P, E155D, H44Q, F191S, I303N, T104K, G119W, Q276P, P14A, L59P, N122I, L17R, A115V, P14S, G11W, S299C, L22W, F364L, R45W, N241Y, Y70C, L75R, N201H, G334V, I121R, P87H, Y230D, V124D, L216R, D187Y, P87L, S179A, G170V, Y289C, A358P, V293M, S178T, G287V, P67L, A205D, W140L, N154T, P292R, V293E, G339V, S275Y, M361T, Y150C, V60E, A343D, W140R, S204Y, F364S, V193G, F83Y, V86G, H335R, A99V, P292Q, Y151S, L321P, R213L, I211S, N89T, E161K, F320C, N180K, S147T, F10I, G263V, P292A, C245Y, I282S, P166L, F364C, F288C, G340S, P166T, I282T, P292S, I303F, E161V, I278T, A111T, G197V, A153P, P166H, C245F, R213C, L64R, M361V, H335Q, T104R, H335N, S344P, S285C, P166S, L216P, F320I, W140G, V338E, G206V, V66F, L228P, F364V, F195S, S43R, Q233P, W140C, Y279H, V188G, R61W, R298I, A146D, D283H, L63W, I303S, M42R, C224Y, S285T, V209E, F320L, F364I, V174L, L357Q, R298S, I307S, V103E, C175Y, E317D, R298T, A106T, I341N, L216Q, N180Y, E317G, T139P, V172G, D163V, A112E, S285A, A324D, E366V, I282F, H158Y, F181C, Y279D, A359P, F191V, N347Y, I281F, S275T, K274I, P12T, Y279F, I303M, G92V, P292L, P166A, R213S, V293A, M177K, V186G, R298K, D163Y, N328I, I134L, V210E, V60G, R298G, V202G, Y279S, D353A, G206E, V21E, I134T, N89S, A264D, C80R, T277P, D100Y, F49S, Q233R, F320Y, F162V, G197S, G262V, Y230S, I134N, A15D, K94I, H368R, Y70H, I134S, I307T, D353Y, A358D, E114K, V290G, V160M, V160G, F118S, Q286P, S204P, V124G, T56R, H158R, M3291, N347D, E161D, F288S, F162I, I248S, K333N, N180H, L182Q, R61L, F10V, T104I, A15P, F288V, K33I, W257S, K274E, M267K, N302D, I281N, F191L, G79A, N347T, D100G, I211T, F305L, F83C, E366K, I303T, L182P, C291S, V186E, V293L, M329V, L316S, I281S, F162S, D163H, D100V, F288I, M267R, A348G, D283V, A99G, S204F, P67R, R5G, P225L, Y230H, L17M, V124F, F162C, V102L, I134F, I278F, G77E, V21G, V160A, V209G, F288L, F360S, C224R, F181L, Y230C, N89H, S189F, I282M, F191C, D314A, L75V, F10C, S178C, S20L, T56P, P67A, R306G, N69I, I258T, I134M, K190E, E57K, S179T, P126A, F305Y, H158L, H335Y, I341L, F305V, E317K, T277R, F83I, P87S, P244L, F83V, F49C, D187G, D283G, L316V, P4L, D100A, K190N, A15V, K356R, A220D, E280K, S108T, Y32D, G263R, V354G, S84T, M361L, I307F, V250F, P225H, F74S, E268V, E114A, E366A, S299T, N284I, A234P, Y101H, D353G, V168D, G218W, P67T, A106P, C152G, P18Q, I194S, C98F, D68Y, A99S, L90F, V202A, N54I, V103M, V290L, P67Q, Y151C, F49L, N58K, Y230N, L327P, V338G, G203C, V102A, T242A, R61Q, F49V, V354A, P135L, K190T, I194F, A109V, H125D, D163A, S46P, Y101F, S337P, K51Q, L59V, N297T, V124A, I307M, A266P, E280V, G159A, C224F, V149A, K116R, N154H, G263D, I52M, Y240D, Y346H, E352V, E229D, F181V, V86F, G197A, D187V, A106G, N54T, F162L, F181I, K333Q, D136A, P67S, D136G, G339E, D68A, W257C, G262A, K274R, S84I, I248N, G159C, K71N, M42V, V349D, I282L, N328Y, L228R, Q76E, V212G, S84C, R222G, E326Q, V35F, S43I, V7G, S183T, Q76H, K190R, V66L, S130R, Q276E, F305I, L40I, G287R, T104S, A343G, D167V, G235V, A220V, V254D, G77V, V157G, R213H, L239V, S238P, D187H, N302S, S275C, G339A, L217M, A196T, A220P, T208I, D100E, S247C, A264G, E62V, E161Q, V290D, S183C, Q6H, D167Y, H19L, I165L, S299G, S20P, Y32F, C98W, C175S, A153T, I258S, E161G, P137R, N284Y, L216M, D187A, A343S, Y279N, K51R, I123F, V21A, S130T, G36C, G308D, V188F, P126R, D215V, K33R, V207F, D100H, S183Y, N328S, I91V, K127R, L217R, I91F, N143K, N201K, V21L, N297D, G262R, G312V, P135T, N69D, N55I, V60A, N69Y, V250D, I165R, K300T, N201I, K323N, I53N, S20T, Q276L, K190Q, L128V, S204C, D187N, F83L, D215N, D353N, H19Q, G119A, E57D, G334E, A265T, Y32H, I73S, S108A, V35I, G81S, V186M, I165K, S331T, L23I, E317Q, V39L, T139S, P126L, H19D, P18A, V212F, G81C, H158Q, L173P, A348V, L316F, N180I, E57V, G235E, P304A, D145V, E24D, A112T, K323R, Q296P, S43G, I351V, N69K, G334R, R222P, T120S, E28A, E366G, V168I, V72A, D353H, I341S, L93S, L142H, T139N, D31E, P137T, F181Y, N309D, V103A, L75P, A85V, H125P, R222L, D163E, D215H, K333E, G129W, N309I, Q369L, G170A, R306C, S247I, V103L, P18R, N347H, E57G, I25M, P244S, W257G, L325H, I273S, I123V, K300E, I53S, Q82L, L327H, L270R, G36V, A266V, V21M, I282V, K300M, S271G, S189P, A50G, C224S, V124L, I52L, T277A, E62Q, K323Q, D313Y, A171S, N328K, I52T, I165M, K27Q, K16R, E24G, F288Y, L255Q, A220T, F195C, T132R, Q286R, G92A, V188A, Q369R, H125L, E256Q, T56A, E268K, P137Q, N54D, L59I, E57A, S147A, K94E, K51M, S237N, V60L, L253M, N236H, G235A, I211F, K16Q, A65V, D283A, D163G, G287A, P225A, K127N, K33E, F364Y, L59H, V39F, A109G, A171V, V227D, T277I, L217Q, M177T, H19P, F164S, G263C, E223V, L216V, D48N, L217V, E229Q, P295L, E113V, F10L, F164I, I303V, S38G, K323E, N58T, N297H, M267T, R5T, V186A, N58I, K27T, A146P, G308A, K133I, P244T, G252V, C224G, G92S, G81V, V202L, V168L, V290I, T104A, P185A, Y240H, E28Q, L345M, G36R, A198V, S272T, K333I, I341M, G263S, E280G, K274T, I165T, D2A, Q296H, A109D, E114V, Q355H, K318E, V254I, D322V, A153S, C98Y, D215Y, I73N, N241T, N141H, K363N, V35L, T120P, I281V, P225S, V212I, A109S, D68G, K274Q, L221P, D215G, L30F, E24V, L64V, D100N, M177V, L128F, G92C, V160L, A198G, E28D, P87T, S147C, E268A, R5M, V290A, A85T, P225T, S247G, R319G, H19R, L365W, I307V, L142V, E223K, L128R, S183A, T132I, W257R, G312R, P135A, D313A, Q276K, P87A, S204A, S38C, K362R, V207L, V250I, S331Y, T139A, A3D, A65D, E294G, H368P, A146G, S237I, T139I, F164Y, N54Y, K184N, R61G, V102M, V60M, I307L, I52N, K363R, D187E, E317A, V149L, E62A, S130C, A265S, D145E, V202I, N122H, Q82E, L64Q, M267L, L59F, S130G, I248F, D145A, K16E, S237G, D167E, Y150H, Q286L, N297Y, A95E, K300N, S247T, K33Q, I123T, K269E, M251T, A153G, Y230F, P135R, F164V, A171P, D145Y, N328T, L270V, I25V, I121L, S189C, A266S, V35G, C98S, M301T, V212L, V7M, D167N, I121M, T350I, P295T, A358V, K190M, P126S, Q276H, G287E, A3S, D215E, K127T, N58Y, F164C, Q296E, F118Y, L142I, L40V, G218A, G96V, M267V, N284D, L88I, D167H, V8D, P185L, P244A, T277S, A310V, R5K, L330M, F164L, L217P, A343P, N141S, N58H, D31V, G312E, L270F, S237C, E268G, A324S, F74V, D314Y, D2V, L370I, N55S, I25L, E366D, L345P, A265P, L17V, A315P, K333R, K363E, V254L, Q369P, V86A, T120I, V254A, D48V, Y70F, E268Q, N261T, N309T, P244Q, K269M, G92D, C152S, N141Y, S238Y, G218R, N9S, V232L, I258L, N284S, E256D, S84N, Q369H, A146S, K231E, V254G, H368D, L253W, E256V, G262E, R306S, Q233K, I278M, E294V, V174A, N261I, S43C, K71R, S138C, L325P, D322G, D2G, A358T, A106V, K269N, V232E, E114G, F360I, P295A, L253V, V210G, V124I, I123M, V207G, I278V, K318I, F246S, E113D, A3T, K27M, S46L, L327R, G218E, L88S, A65G, Q286E, Q296K, K184R, P18S, Q6R, F360Y, D48A, A146V, I273L, L332V, N141D, V168A, V232G, I53V, L59R, T277K, N328D, Q233H, S272C, I52S, L75Q, S130N, L327F, N141K, D322N, S138I, D314H, Y151H, L142R, K274N, G96A, L40F, A234S, K184E, N58S, L182V, E294K, M301K, P12H, L327V, K362T, I91L, G81A, I73T, I351S, K362Q, L40H, L330V, Q26L, N309Y, V212A, A266D, G308S, F360C, A111G, D68H, S247N, A50D, S331P, K363Q, E326A, K362N, K33N, E229V, Y249D, N261K, K27N, G170R, K311N, A220S, P4A, I278L, A112V, I273N, T132P, L221F, S237T, D214H, L142P, G218V, G36D, I341F, E268D, L142F, G308R, A315S, D31N, A264P, R306L, S47A, G34V, I211V, I258N, E223Q, N284H, A50S, P295S, Q6E, A358G, L321H, D214A, A205S, V188I, Y240F, I273M, F118L, D313E, D314G, G36A, I273V, N122T, R319I, A65S, C291G, G34R, F219Y, S271R, Q276R, M42L, G339R, E57Q, V168G, P18L, K169N, D322E, A310P, E113A, V66I, K259I, S331A, N260H, L29S, I123L, I52F, S337T, M367R, L90V, K231Q, F219V, K127Q, D214G, P126H, A324T, I194T, Y249S, Q76P, I37S, D145N, G77A, D314E, E294Q, K127E, D313G, S138N, A324G, D48H, Y240C, D68N, G129A, I281L, V202F, L365S, D322A, E280D, I273F, K110R, K27E, E161A, N201S, F219L, V193A, L239W, K363T, K269T, I248V, N143D, D31G, Q355L, E280Q, A196V, L93W, W257L, K323T, Q82H, V250L, S272Y, G252D, D313H, P304R, L270H, Q6P, A264S, I211M, L128I, N309K, V86I, N122D, N55Y, M367V, H19N, V172E, H368N, K184T, K16T, D136N, R97K, A266T, R336T, N260I, N260Y, D2N, S272F, I351L, M329L, L321V, A343V, G129E, N54K

86. The method of claim 1, wherein the dietary supplementation comprises an effective amount of L-serine.

87. The method of claim 1, wherein the effective amount comprises 100 to 950 mg/kg/day of the L-serine.

88. The method of claim 1, wherein the dietary supplementation comprises an effective amount glycine.

89. The method of claim 1, wherein the effective amount comprises 200 to 400 mg/kg/day of the glycine.
